(19) United States
(12) Patent Application Publication
(10) Pub. No.: US 2026/0176314 A1
(43) Pub. Date: Jun. 25, 2026

(54) PROTEINS, METHODS, AND SYSTEMS FOR SUBCELLULAR LOCALIZATION OF PROTEINS FOR POST-TRANSLATION MODIFICATION

(71) Applicant: BETTER DAIRY LIMITED, London (GB)

(72) Inventors: Bernd Alfons Gerhartz, London (GB); Kate Royle, London (GB); Christian Arenas López, Brighton (GB); Camila Aparecida Cotrim, Ipswich (GB); Agata Giardina, London (GB)

(21) Appl. No.: 19/251,289

(22) Filed: Jun. 26, 2025

Related U.S. Application Data
(63) Continuation of application No. PCT/EP2023/088026, filed on Dec. 29, 2023.
(60) Provisional application No. 63/436,041, filed on Dec. 29, 2022.

(51) Int. Cl. C07K 14/52; A23C 11/06; C12N 1/165; C12N 9/12; C12R 1/84; C12R 1/87

(52) U.S. Cl. CPC C07K 14/52; A23C 11/065; C12N 1/165; C12N 9/12; C07K 2319/05; C12R 2001/84; C12R 2001/87; C12Y 207/11001

(57) ABSTRACT

The present disclosure provides for proteins, methods, systems for directing enzymes for generating post-translational modifications on proteins to a particular subcellular location. Specifically, kinases for phosphorylation of proteins containing one or more sites susceptible to phosphorylation are disclosed, also with methods and systems for phosphorylating proteins of interest, such as proteins having nutritional and therapeutic uses.

### CROSS-REFERENCE TO RELATED APPLICATIONS

This application is a continuation of International Application No. PCT/EP2023/088026, filed Dec. 29, 2023, which claims the benefit of U.S. Provisional Application No. 63/436,041 filed Dec. 29, 2022, the contents of which are incorporated by reference in their entirety.

### REFERENCE TO A SEQUENCE LISTING

The instant application contains a Sequence Listing which has been submitted electronically in XML file format and is hereby incorporated by reference in its entirety. Said XML copy, created on Dec. 24, 2025, is named 64309-707_301_SL.xml, and is 510,924 bytes in size.

### BACKGROUND

Kinases that act to phosphorylate proteins can be used to produce therapeutic and nutritional protein products. For instance, the construction and stabilization of protein micelles—aggregates of proteins that provide structural properties to protein products—often involves phosphorylation of proteins by one or more kinases. These protein micelles may also contain one or more minerals, such as calcium and phosphorous (e.g., calcium phosphate). The formation of protein micelles, and the phosphorylation of proteins by protein kinases that leads to micelle formation, can contribute greatly to the therapeutic and nutritional properties of protein products. The use of recombinant non-animal host cells to produce phosphorylated proteins derived from animals—which boasts benefits in speed, scale, and cost—may be limited in part to the absence of an appropriate kinase within the secretory pathway of the recombinant non-animal cell. Furthermore, the expression of animal-derived kinases that retain their full phosphorylating functionality in non-animal cells in recombinant non-animal cells can be challenging. Thus, the use of recombinant non-animal cells to produce phosphorylated proteins derived from animals has found limited success.

### SUMMARY

Disclosed herein is a recombinant host cell engineered to express a non-naturally occurring polypeptide comprising a heterologous serine/threonine kinase, wherein the heterologous serine/threonine kinase is anchored to an intracellular membrane. The heterologous serine/threonine kinase can be a human, bovine, primatial, avian, or reptile serine/threonine kinase or a biologically active portion thereof. The heterologous serine/threonine kinase can be a human serine/threonine kinase or a biologically active portion thereof. The heterologous serine/threonine kinase can be a lemur serine/threonine kinase or a biologically active portion thereof. The heterologous serine/threonine kinase can comprise an amino acid sequence having at least 75%, 80%, 85%, 90%, 95%, or 100% sequence identity to the SEQ ID NO. 7. The heterologous serine/threonine kinase can consist of an amino acid sequence having at least 75%, 80%, 85%, 90%, 95%, or 100% sequence identity to the SEQ ID NO. 7.

Described herein are non-naturally occurring polypeptides comprising a serine/threonine kinase coupled to a heterologous domain capable of anchoring the serine/threonine kinase to an intracellular membrane.

In some embodiments, the serine/threonine kinase comprises a human, bovine, primatial, avian, or reptile serine/threonine kinase. In some embodiments, the serine/threonine kinase comprises a human serine/threonine kinase. In some embodiments, the human serine/threonine kinase comprises a Fam20c kinase. In some embodiments, the human serine/threonine kinase is a Fam20c kinase. In some embodiments, the serine/threonine kinase comprises an amino acid sequence having at least 75%, 80%, 85%, 90%, 95%, or 100% sequence identity to the amino acid sequence set forth in SEQ ID NO: 7.

In some embodiments, the serine/threonine kinase comprises a truncation at an N-terminal end of up to 10 amino acids, 20 amino acids, 30 amino acids, 40 amino acids, 50 amino acids, 60 amino acids, 70 amino acids, 80 amino acids, 90 amino acids, or 100 amino acids. In some embodiments, the serine/threonine kinase comprises a truncation at an N-terminal end of up to 92 amino acids.

In some embodiments, the serine/threonine kinase consists of an amino acid sequence having at least 75%, 80%, 85%, 90%, 95%, or 100% sequence identity to the amino acid sequence set forth in SEQ ID NO: 7. In some embodiments, the serine/threonine kinase consists of the amino acid sequence set forth in SEQ ID NO: 7. In some embodiments, the serine/threonine kinase comprises an amino acid sequence having at least 75%, 80%, 85%, 90%, 95%, or 100% sequence identity to the amino acid sequence set forth in SEQ ID NO: 142. In some embodiments, the serine/threonine kinase consists of an amino acid sequence having at least 75%, 80%, 85%, 90%, 95%, or 100% sequence identity to the amino acid sequence set forth in SEQ ID NO: 142. In some embodiments, the serine/threonine kinase consists of the amino acid sequence set forth in SEQ ID NO: 142. In some embodiments, the serine/threonine kinase comprises an amino acid sequence having at least 75%, 80%, 85%, 90%, 95%, or 100% sequence identity to the amino acid sequence set forth in SEQ ID NO: 143. In some embodiments, the serine/threonine kinase consists of an amino acid sequence having at least 75%, 80%, 85%, 90%, 95%, or 100% sequence identity to the amino acid sequence set forth in SEQ ID NO: 143. In some embodiments, the serine/threonine kinase consists of the amino acid sequence set forth in SEQ ID NO: 143. In some embodiments, the serine/threonine kinase comprises an amino acid sequence having at least 75%, 80%, 85%, 90%, 95%, or 100% sequence identity to the amino acid sequence set forth in SEQ ID NO: 144. In some embodiments, the serine/threonine kinase consists of an amino acid sequence having at least 75%, 80%, 85%, 90%, 95%, or 100% sequence identity to the amino acid sequence set forth in SEQ ID NO: 144. In some embodiments, the serine/threonine kinase consists of the amino acid sequence set forth in SEQ ID NO: 144.

In some embodiments, the heterologous domain is coupled to the N-terminus of the serine/threonine kinase. In some embodiments, the heterologous domain is coupled to the C-terminus of the serine/threonine kinase. In some embodiments, the heterologous domain comprises a fragment of a Type I or a Type II transmembrane protein. In some embodiments, the fragment of the Type I or Type II transmembrane protein comprises a Type I or a Type II transmembrane domain. In some embodiments, the heterologous domain comprises a fragment of a Type II transmembrane protein. In some embodiments, the fragment of the Type II transmembrane protein comprises a Type II transmembrane domain. In some embodiments, the fragment of the Type II transmembrane protein comprises the Type II transmembrane protein with a truncation at a C-terminal end. In some embodiments, the fragment of the Type II transmembrane protein is 160 amino acids or less, is 100 amino acids or less, or is 60 amino acids or less. In some embodiments, the fragment of the Type II transmembrane protein is derived from Saccharomyces cerevisiae or Pichia pastoris. In some embodiments, the fragment of the Type II transmembrane protein is derived from Saccharomyces cerevisiae. In some embodiments, the fragment of the Type II transmembrane protein is derived from Pichia pastoris. In some embodiments, the heterologous domain comprises a multi-pass transmembrane domain.

In some embodiments, the heterologous domain comprises an amino acid sequence having at least 75%, 80%, 85%, 90%, 95%, or 100% sequence identity to any one of the amino acid sequences set forth in SEQ ID NOs: 23-85. In some embodiments, the heterologous domain consists of an amino acid sequence having at least 75%, 80%, 85%, 90%, 95%, or 100% sequence identity to any one of the amino acid sequences set forth in SEQ ID NOs: 23-85. In some embodiments, the heterologous domain comprises an amino acid sequence having at least 75%, 80%, 85%, 90%, 95%, or 100% sequence identity to the amino acid sequence set forth in SEQ ID NO: 71. In some embodiments, the heterologous domain consists of an amino acid sequence having at least 75%, 80%, 85%, 90%, 95%, or 100% sequence identity to the amino acid sequence set forth in SEQ ID NO: 71. In some embodiments, the non-naturally occurring polypeptide comprises an amino acid sequence having at least 75%, 80%, 85%, 90%, 95%, or 100% sequence identity to any one of the amino acid sequences set forth in SEQ ID NOs: 93, 95-98, 100-109, 111-114, 116, 117, 119-125, 127-130, 132, 133, 136-141, and 154. In some embodiments, the non-naturally occurring polypeptide comprises an amino acid sequence having at least 75%, 80%, 85%, 90%, 95%, or 100% sequence identity to the amino acid sequence set forth in SEQ ID NO: 133. In some embodiments, the non-naturally occurring polypeptide consists of an amino acid sequence having at least 75%, 80%, 85%, 90%, 95%, or 100% sequence identity to any one of the amino acid sequences set forth in SEQ ID NOs: 93, 95-98, 100-109, 111-114, 116, 117, 119-125, 127-130, 132, 133, 136-141, and 154. In some embodiments, the non-naturally occurring polypeptide consists of an amino acid sequence having at least 75%, 80%, 85%, 90%, 95%, or 100% sequence identity to the amino acid sequence set forth in SEQ ID NO: 133.

In some embodiments, the non-naturally occurring polypeptide further comprises a tag. In some embodiments, the tag comprises a FLAG tag. In some embodiments, the tag comprises a MYC tag.

In some embodiments, the heterologous domain comprises a Golgi retention sequence.

In some embodiments, the intracellular membrane comprises a Golgi apparatus membrane, an endoplasmic reticulum membrane, a nuclear membrane, or a mitochondrial membrane. In some embodiments, the intracellular membrane comprises an endoplasmic reticulum membrane. In some embodiments, the intracellular membrane is a Golgi apparatus membrane. In some embodiments, the serine/threonine kinase is anchored such that a kinase domain of the serine threonine kinase is positioned on the luminal side of the Golgi apparatus membrane.

Described herein are nucleic acid molecules encoding for any one of the non-naturally occurring polypeptides described herein.

Described herein are expression vectors encoding for (i) any one of the non-naturally occurring polypeptides described herein and (ii) a secretory protein.

Described herein are recombinant host cells comprising: (i) any one of the non-naturally occurring polypeptides described herein, wherein the serine/threonine kinase is heterologous to the recombinant host cell; (ii) any one of the nucleic acid molecules described herein.

In some embodiments, the recombinant host cell comprises a fungal host cell, a bacterial host cell, an algal host cell, or a plant host cell. In some embodiments, the recombinant host cell comprises a bacterium. In some embodiments, the bacterium is Escherichia coli or Bacillus subtilis.

In some embodiments, the recombinant host cell comprises a eukaryotic host cell. In some embodiments, the eukaryotic host cell comprises a fungus. In some embodiments, the fungus comprises Aspergillus, Candida, Fusarium, Hansenula, Kluyveromyces, Pichia, Penicillium, Saccharomyces, Tetrahymena, Thermothelomyces, Trichoderma, Yarrowia, or Zygosaccharomyces. In some embodiments, the fungus comprises a yeast cell. In some embodiments, the fungus comprises Kluyveromyces lactis. In some embodiments, the fungus comprises Pichia pastoris. In some embodiments, the fungus comprises a filamentous fungus. In some embodiments, the filamentous fungus comprises Aspergillus or Thermothelomyces heterothallica.

In some embodiments, the serine/threonine kinase is anchored to the intracellular membrane via a lipid. In some embodiments, the lipid is covalently attached to the serine/threonine kinase. In some embodiments, the serine/threonine kinase and the lipid form a prenylated protein. In some embodiments, the lipid covalently attached to the serine/threonine kinase comprises a fatty acyl group. In some embodiments, the lipid and the serine/threonine kinase form a glycosylphosphatidylinositol-linked protein.

Described herein are kits comprising a nucleic acid molecule encoding for any one of the non-naturally occurring polypeptides described herein and a nucleic acid molecule encoding for a secretory protein. In some embodiments, the kit further comprises a host cell.

Described herein are methods comprising using the serine/threonine kinase of any one of the recombinant host cells described herein to phosphorylate a secretory protein, thereby generating a phosphorylated secretory protein. In some embodiments, the method further comprises harvesting the phosphorylated secretory protein from the recombinant host cell, thereby generating a harvested phosphorylated secretory protein. In some embodiments, the method further comprises using the harvested phosphorylated secretory protein for manufacture of a food product.

In some embodiments, the food product is a dairy product. In some embodiments, the food product is a dairy substitute. In some embodiments, the food product is a cheese.

Described herein are fungal cells comprising a heterologous kinase, wherein the heterologous kinase is a Fam20c member. In some embodiments, the heterologous kinase is a human Fam20c member. In some embodiments, the Fam20c member is a truncated Fam20c member.

Described herein are caseins phosphorylated at one or more amino residues that are not phosphorylated in nature. In some embodiments, the casein is an αS1-casein. In some embodiments, the αS1-casein is phosphorylated at S56, S61, S63, T64, S79, S81, S82, S83, S90, S103, or S130. In some embodiments, the αS1-casein is phosphorylated at S56, S79, S81, S82, S83, or S103. In some embodiments, the αS1-casein is phosphorylated at S56. In some embodiments, the αS1-casein is phosphorylated at S56, S61, S63, T64, S79, S81, S82, S83, S90, S103, and S130. In some embodiments, the phosphorylated casein is an αS2-casein. In some embodiments, the αS2-casein is phosphorylated at S23, S24, S25, S46, S52, S71, S72, S73, S76, T145, S146, S150, S158, T159, or T163. In some embodiments, the αS2-casein is phosphorylated at S23, S24, S25, S52, S71, S72, S73, or S76. In some embodiments, the αS2-casein is phosphorylated at S23, S52, S71, S72, S73, or S76. In some embodiments, the αS2-casein is phosphorylated at S23, S52, S71, S72, S73, and S76. In some embodiments, the αS2-casein is phosphorylated at S23, S24, S25, S46, S52, S71, S72, S73, S76, T145, S146, S150, S158, and T159. In some embodiments, the phosphorylated casein is a β-casein. In some embodiments, the β-casein is phosphorylated at S30, S32, S33, S34, S37, T39, S50, or T56. In some embodiments, the β-casein is phosphorylated at S30, S32, S33, S34, S37, or T39. In some embodiments, the β-casein is phosphorylated at S37. In some embodiments, the β-casein is phosphorylated at S30, S32, S33, S34, S37, T39, S50, and T56. In some embodiments, the phosphorylated casein is κ-casein.

In some embodiments, the casein is phosphorylated at two or more amino acid residues that are not phosphorylated in nature. In some embodiments, the casein is phosphorylated at three or more amino acid residues that are not phosphorylated in nature. In some embodiments, the casein is phosphorylated at four or more amino acid residues that are not phosphorylated in nature. In some embodiments, the casein is phosphorylated at five or more amino acid residues that are not phosphorylated in nature. In some embodiments, the casein is phosphorylated at six or more amino acid residues that are not phosphorylated in nature.

Described herein is an engineered food product comprising the casein described herein. In some embodiments, the food product is a dairy product. In some embodiments, the food product is dairy substitute. In some embodiments, the food product is a cheese. Described herein are methods of producing the phosphorylated casein described herein. In some embodiments, the method comprises using the non-naturally occurring polypeptide described herein to phosphorylate a casein.

The heterologous serine/threonine kinase can be a primatial serine/threonine kinase or a biologically active portion thereof. The heterologous serine/threonine kinase can be a lemur serine/threonine kinase or a biologically active portion thereof. The heterologous serine/threonine kinase can comprise an amino acid sequence having at least about 75%, 80%, 85%, 90%, 95%, or 100% sequence identity to the sequence set forth in SEQ ID NO. 6. The heterologous serine/threonine kinase can consist of an amino acid sequence having at least about 75%, 80%, 85%, 90%, 95%, or 100% sequence identity to the sequence set forth in SEQ ID NO. 6.

The heterologous serine/threonine kinase can be an avian serine/threonine kinase or a biologically active portion thereof. The heterologous serine/threonine kinase can be a sandgrouse serine/threonine kinase or a biologically active portion thereof. The heterologous serine/threonine kinase can comprise an amino acid sequence having at least about 75%, 80%, 85%, 90%, 95%, or 100% sequence identity to the sequence set forth in SEQ ID NO. 8. The heterologous serine/threonine kinase can consist of an amino acid sequence having at least about 75%, 80%, 85%, 90%, 95%, or 100% sequence identity to the sequence set forth in SEQ ID NO. 8.

In some embodiments, the heterologous serine/threonine kinase is less than 400 amino acids in length. In some embodiments, the heterologous serine/threonine kinase is less than 360 amino acids in length. In some embodiments, the heterologous serine/threonine kinase is less than 350 amino acids in length. In some embodiments, the heterologous serine/threonine kinase is less than 300 amino acids in length. In some embodiments, the heterologous serine/threonine kinase is less than 200 amino acids in length. In some embodiments, the heterologous serine/threonine kinase is less than 140 amino acids in length.

In some embodiments, the heterologous serine/threonine kinase or a biologically active portion thereof is a heterologous reptile serine/threonine kinase or a biologically active portion thereof. In some embodiments, the heterologous reptile serine/threonine kinase is a reptile Fam20c protein or a biologically active portion thereof. In some embodiments, the reptile Fam20c protein is a snake Fam20c protein or a biologically active portion thereof. The snake Fam20c protein or a biologically active portion thereof can comprise an amino acid sequence having at least about 80%, 90%, or 95% sequence identity to the sequence set forth in SEQ ID NO: 3. The snake Fam20c protein can comprise an amino acid sequence of SEQ ID NO: 3. The heterologous serine/threonine kinase can comprise a sequence with at least 80%, at least 90%, at least 95% or at least 99% sequence identity to the sequence set forth in SEQ ID NO: 3 but does not include a sequence of greater than 20 amino acids in length having 90% sequence identity to any region of the sequence set forth in SEQ ID NO: 9.

In some embodiments, the heterologous serine/threonine kinase disclosed herein can be anchored to an intracellular membrane via a polypeptide. The heterologous serine/threonine kinase can be attached to the polypeptide at the N-terminus or C-terminus of the heterologous serine/threonine kinase. The polypeptide can be a Type 1, Type 2, or a multi-pass transmembrane domain. The polypeptide can comprise a Golgi retention sequence. The polypeptide can comprise the sequence set forth in any one of SEQ ID NOs: 21-22. In some embodiments, the polypeptide of any one of SEQ ID NOs: 21-22 is at the C-terminus of the polypeptide. The polypeptide can comprise an amino acid sequence having at least about 75%, 80%, 85%, 90%, 95% sequence identity to the sequence set forth in any one of SEQ ID NOs: 23-85. The polypeptide can comprise an amino acid sequence of any one of SEQ ID NOs: 23-85.

In some embodiments, the heterologous serine/threonine kinase disclosed herein can be anchored to an intracellular membrane via a lipid. In some embodiments, the lipid is covalently attached to the heterologous serine/threonine kinase. In some embodiments, the heterologous serine/threonine kinase and the lipid form a prenylated protein. The lipid can comprise a fatty acyl group. In some embodiments, the lipid attachment to the heterologous serine/threonine kinase can form a glycosylphosphatidylinositol-linked protein. The intracellular membrane can be an endoplasmic reticulum (ER) membrane, a nuclear membrane, a mitochondrial membrane, or a Golgi apparatus membrane. The heterologous serine/threonine kinase can be anchored to the Golgi apparatus membrane such that a kinase domain of the heterologous serine/threonine kinase is positioned on the luminal side of the Golgi apparatus membrane.

The serine/threonine kinase can further comprise a purification, identification, or solubility enhancer tag. The purification and/or identification tag can be a FLAG tag. The solubility enhancer tag can be a SUMO tag, a TRX tag, a MBP tag, a MISTIC tag, a NusA tag, a FLAG-SUMO tag, a FLAG-TRX tag, a FLAG-MBP tag, a FLAG-MISTIC tag, or a FLAG-NusA tag. The purification and/or identification tag can be a FLAG tag that comprises the sequence set forth in SEQ ID NO. 10. The solubility enhancer tag can be a SUMO tag that comprises the sequence set forth in SEQ ID NO. 11. The solubility enhancer tag can be a TRX tag that comprises the sequence set forth in SEQ ID NO. 12. The solubility enhancer tag can be a MBP tag that comprises the sequence set forth in SEQ ID NO. 13. The solubility enhancer tag can be a MISTIC tag that comprises the sequence set forth in SEQ ID NO. 14. The solubility enhancer tag can be a NusA tag that comprises the sequence set forth in SEQ ID NO. 15. The solubility enhancer tag can be a FLAG-SUMO tag that comprises the sequence set forth in SEQ ID NO. 16. The solubility enhancer tag can be a FLAG-TRX tag that comprises the sequence set forth in SEQ ID NO. 17. The solubility enhancer tag can be a FLAG-MBP tag that comprises the sequence set forth in SEQ ID NO. 18. The solubility enhancer tag can be a FLAG-MISTIC tag that comprises the sequence set forth in SEQ ID NO. 19. The solubility enhancer tag can be a FLAG-NusA tag that comprises the sequence set forth in SEQ ID NO. 20.

Described herein is a recombinant host cell, in some embodiments, that can be configured to express a heterologous secretory susceptible to phosphorylation by the serine/threonine kinase. The heterologous secretory protein can be a therapeutic protein or a nutritional protein. The nutritional protein can be a dairy protein. The dairy protein can be a casein or a portion thereof. The casein can be αs1-casein, αs2-casein, β-casein, or κ-casein or a portion thereof. The dairy protein can be glycomacropeptide or a portion thereof. The dairy protein can be osteopontin or a portion thereof. The dairy protein can be lactoferrin or a portion thereof. The nutritional protein can be an egg white protein. The egg white protein can be ovalbumin or a portion thereof.

The heterologous secretory protein comprising one or more phosphorylation sites can be a component of a protein complex. The protein complex can comprise a first protein and a second protein. The first protein can be the heterologous protein comprising one or more phosphorylation sites susceptible to phosphorylation by a serine/threonine kinase. The second protein can be the heterologous protein comprising one or more phosphorylation sites susceptible to phosphorylation by a serine/threonine kinase. The protein complex can further comprise four proteins, wherein each of the proteins of the protein complex comprises one or more phosphorylation sites susceptible to phosphorylation by a serine/threonine kinase.

The recombinant host cell can be a fungal recombinant host cell, a bacterial recombinant host cell, an algal recombinant host cell, or a plant recombinant host cell. The recombinant host cell can be a bacterium. The bacterium can be Escherichia coli or Bacillus subtilis. The recombinant host cell can be a eukaryotic cell. The recombinant host cell can be a fungus. The fungus can be Aspergillus, Candida, Fusarium, Hansenula, Kluyveromyces, Pichia (synonym Komagataella), Penicillium, Saccharomyces, Tetrahymena, Trichoderma, Yarrowia, or Zygosaccharomyces. The fungus can be yeast. The yeast cell can be Kluyveromyces lactis or Pichia pastoris (synonym Komagataella phaffi). The fungus can be a filamentous fungus. The filamentous fungus can be Aspergillus.

Described herein is a recombinant host cell comprising an expression vector. The expression vector can comprise a nucleic acid sequence encoding the heterologous serine/threonine kinase. Described herein, in some embodiments, is a recombinant host cell comprising an expression vector wherein the expression vector can comprise a nucleic acid sequencing encoding (1) the heterologous serine/threonine kinase and (2) a polypeptide attached to the heterologous serine/threonine kinase.

Described herein is a composition comprising a heterologous serine/threonine kinase conjugated to a domain for anchoring the serine/threonine kinase to an intracellular membrane. In some embodiments, the composition can be expressed in recombinant host cells as previously described. The domain can be a polypeptide. The polypeptide can be a Type 1, Type 2, or a multi-pass transmembrane domain. The polypeptide can comprise a Golgi retention sequence. The polypeptide can comprise the sequence set forth in any one of SEQ ID NOs: 21-22. In some embodiments, the polypeptide of any one of SEQ ID NOs: 21-22 is at the C-terminus of the polypeptide. The polypeptide can comprise an amino acid sequence having at least about 75%, 80%, 85%, 90%, 95% sequence identity to the sequence set forth in any one of SEQ ID NOs: 23-85. The polypeptide can comprise an amino acid sequence of any one of SEQ ID NOs: 23-85. The domain can be a lipid. In some embodiments, the lipid is covalently attached to the heterologous serine/threonine kinase. In some embodiments, the heterologous serine/threonine kinase and the lipid form a prenylated protein. The lipid can comprise a fatty acyl group. In some embodiments, the lipid attachment to the heterologous serine/threonine kinase can form a glycosylphosphatidylinositol-linked protein. The intracellular membrane can be an endoplasmic reticulum (ER) membrane, a nuclear membrane, a mitochondrial membrane, or a Golgi apparatus membrane. The heterologous serine/threonine kinase can be anchored to the Golgi apparatus membrane such that a kinase domain of the heterologous serine/threonine kinase is positioned on the luminal side of the Golgi apparatus membrane.

Described herein is a secretory protein phosphorylated by the composition. Described herein, in some embodiments, is a food product comprising the secretory protein phosphorylated by the composition. The food product can be a dairy product or a dairy substitute. The food product can be a cheese.

Described herein is the use of the previously recombinant host cells to manufacture a food product. Described herein is the use of the previously recombinant host cells to manufacture a food product, wherein the food product can be a dairy product or dairy substitute. Described herein is the use of the previously recombinant host cells to manufacture a food product, wherein the food product can be a cheese.

Described herein is a method of manufacturing a phosphorylated secreted protein, the method comprising: expressing, in a cell population comprising a plurality of recombinant host cells, (1) a heterologous serine/threonine kinase conjugated to a domain for anchoring the serine/threonine kinase to an intracellular membrane and (2) a heterologous secreted protein comprising one or more phosphorylation sites susceptible to phosphorylation by the serine/threonine kinase. The method may further comprise (1) transforming the cell population with a first vector encoding the serine/threonine kinase under control of a first inducible promoter, and (2) transforming the cell population with a second vector encoding the secreted protein comprising one or more phosphorylation sites susceptible to phosphorylation by the serine/threonine kinase under control of a second inducible promoter. The method may further comprise transforming the cell population with a vector encoding both (1) the serine/threonine kinase under control of a first inducible promoter and (2) the secreted protein comprising one or more phosphorylation sites susceptible to phosphorylation by the serine/threonine kinase under control of a second inducible promoter. The method may further comprise incorporating into the recombinant host cell genome a first fragment of DNA encoding the serine/threonine kinase under control of a first promoter and a second fragment of DNA encoding the secreted protein comprising one or more phosphorylation sites susceptible to phosphorylation by the serine/threonine kinase under control of a second promoter. The method may further comprise incorporating into the recombinant host cell genome a fragment of DNA encoding both (1) the serine/threonine kinase under control of a first promoter and (2) the secreted protein comprising one or more phosphorylation sites susceptible to phosphorylation by the serine/threonine kinase under control of a second promoter.

The plurality of recombinant host cells used of the method described herein can comprise any of the previously described recombinant host cells. The domain of the method described herein can be a polypeptide. The polypeptide of the method described herein can be a Type 1, Type 2, or a multi-pass transmembrane domain. The polypeptide of the method described herein can comprise a Golgi retention sequence. The polypeptide of the method described herein can comprise the sequence set forth in any one of SEQ ID NOs: 21-22. In some embodiments, the polypeptide of any one of SEQ ID NOs: 21-22 is at the C-terminus of the polypeptide of the method described herein. The polypeptide of the method described herein can comprise an amino acid sequence having at least about 75%, 80%, 85%, 90%, 95% sequence identity to the sequence set forth in any one of SEQ ID NOs: 23-85. The polypeptide of the method described herein can comprise an amino acid sequence of any one of SEQ ID NOs: 23-85. The domain of the method described herein can be a lipid. In some embodiments, the lipid of the method described herein is covalently attached to the heterologous serine/threonine kinase. In some embodiments, the heterologous serine/threonine kinase and the lipid of the method described herein form a prenylated protein. The lipid of the method described herein can comprise a fatty acyl group. In some embodiments, the lipid attachment to the heterologous serine/threonine kinase of the method described herein can form a glycosylphosphatidylinositol-linked protein. The intracellular membrane of the method described herein can be an endoplasmic reticulum (ER) membrane, a nuclear membrane, a mitochondrial membrane, or a Golgi apparatus membrane. The heterologous serine/threonine kinase of the method described herein can be anchored to the Golgi apparatus membrane such that a kinase domain of the heterologous serine/threonine kinase is positioned on the luminal side of the Golgi apparatus membrane.

Expressing the heterologous serine/threonine kinase in the method described herein can comprise culturing the recombinant host cells in the media. The media can be BMGY media. The first promoter of the method described herein can be an inducible promoter or a constitutive promoter. The second promoter of the method described herein can be an inducible promoter or a constitutive promoter. The first promoter and the second promoter of the method described herein can be the same.

The inducible promoter can be AOX1, ADH3, DAS, FLD1, THI11, GTH1, CUP1, LRA3, LRA4, amyB, bphA, catA, gloaA, and thiA. The constitutive promoter can be GAP, TEF1, YPT1, PGK1, adhA, gdhA, pkgA, and pkiA. Expressing the heterologous serine/threonine kinase can comprise adding an induction agent to the media. The induction agent can be methanol, IPTG, ethanol, maltose, starch, xylose, thiamine, copper, quinic acid, nitrate, glucose, galactose, saccharides, H2O2, CaCO3 or benzoic acid. The heterologous serine/threonine kinase can be induced by light, blue light, low pH, iron starvation or copper depletion. The method may further comprise culturing the plurality of recombinant cells at a temperature between 20 degrees Celsius and 40 degrees Celsius. The method may further comprise harvesting the phosphorylated protein by centrifuging the plurality of recombinant host cells and collecting the supernatant.

Described herein is an expression vector encoding (1) a heterologous serine/threonine kinase conjugated to a domain for anchoring the serine/threonine kinase to an intracellular membrane and (2) a heterologous secreted protein comprising one or more phosphorylation sites susceptible to phosphorylation by the serine/threonine kinase. In some embodiments, the heterologous serine/threonine kinase conjugated to a domain for anchoring the serine/threonine kinase to an intracellular membrane comprises the aforementioned composition. In some embodiments, the secreted protein of the expression vector is a therapeutic protein. In some embodiments, the secreted protein of the expression vector is a nutritional protein. In some embodiments, the nutritional protein is a dairy protein. The nutritional protein can be a dairy protein. The dairy protein can be a casein or a portion thereof. The casein can be αs1-casein, αs2-casein, β-casein, or κ-casein or a portion thereof. The dairy protein can be glycomacropeptide or a portion thereof. The dairy protein can be osteopontin or a portion thereof. The dairy protein can be lactoferrin or a portion thereof. The nutritional protein can be an egg white protein. The egg white protein can be ovalbumin or a portion thereof.

The heterologous secretory protein comprising one or more phosphorylation sites of the expression vector can be a component of a protein complex. The protein complex can comprise a first protein and a second protein. The first protein can be the heterologous protein comprising one or more phosphorylation sites susceptible to phosphorylation by a serine/threonine kinase. The second protein can be the heterologous protein comprising one or more phosphorylation sites susceptible to phosphorylation by a serine/threonine kinase. The protein complex can further comprise four proteins, wherein each of the proteins of the protein complex comprises one or more phosphorylation sites susceptible to phosphorylation by a serine/threonine kinase.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

FIG. 1 shows a cartoon showing alignment of Fam20c protein kinases from snake (SEQ ID NO: 3), lemur (SEQ ID NO: 6), and sandgrouse (SEQ ID NO: 8) as compared to the human Fam20c protein kinase sequence (top, SEQ ID NO: 7). The putative Fam20c kinase domain, as annotated by NCBI, is shown second from the bottom.

FIG. 2 shows an alignment of human (top row, SEQ ID NO: 7), bovine (second row, SEQ ID NO: 2), mouse (third row, SEQ ID NO: 1), sandgrouse (fourth row, SEQ ID NO: 8), lemur (fifth row, SEQ ID NO: 6), and snake (sixth row, SEQ ID NO: 3) Fam20c protein kinase sequences.

FIG. 3 shows an image of a western blot of proteins secreted by transgenic P. pastoris CBS7435 expressing the snake Fam20c Common Garter Snake (FCGS)-3, FCGS-5, or FCGS-8 variants, probed using an anti-FLAG primary antibody conjugated to HRP.

FIG. 4A shows an image of a western blot used to detect extracellular mouse Fam20c protein (SEQ ID NO: 1) extracted from the supernatant portion of transgenic Pichia pastoris CBS7435, probed using an anti-FLAG primary antibody conjugated to HRP.

FIG. 4B shows an image of a western blot used to detect extracellular bovine Fam20c protein (SEQ ID NO: 2) extracted from the supernatant of transgenic Pichia pastoris CBS7435, probed using an anti-FLAG primary antibody conjugated to HRP.

FIG. 4C shows an image of a western blot used to detect intracellular mouse Fam20c protein (SEQ ID NO: 1) extracted from the cell lysate of transgenic Pichia pastoris CBS7435, probed using an anti-FLAG primary antibody conjugated to HRP.

FIG. 4D shows an image of a western blot used to detect intracellular bovine Fam20c protein (SEQ ID NO: 2) extracted from the cell lysate of transgenic Pichia pastoris CBS7435, probed using an anti-FLAG primary antibody conjugated to HRP.

FIG. 4E shows an image of a western blot used to detect intracellular snake Fam20c protein (SEQ ID NO: 3) extracted from the cell lysate of transgenic Pichia pastoris CBS7435, probed using an anti-FLAG primary antibody conjugated to HRP.

FIG. 5 shows an image of a western blot used to detect intracellular expression of soluble and insoluble bovine αS1 casein protein (SEQ ID NO: 4) fractions under the control of the pAOX1 promoter extracted from transgenic PichiaPink™ yeast, probed using an anti-FLAG primary antibody conjugated to HRP.

FIG. 6 shows an image of a western blot used to detect extracellular expression of human osteopontin protein (SEQ ID NO: 5) under the control of the pAOX1 promoter extracted from transgenic Pichia pastoris CBS7435 yeast, probed using an anti-FLAG primary antibody conjugated to HRP.

FIGS. 7A-7D show a schematic of an approach to engineering human serine/threonine protein kinase Fam20c (huFam20c) for fungal expression. FIG. 7A shows a structure of a Type II Transmembrane domain protein. FIG. 7B depicts a description of huFam20c and three truncations.

FIG. 7C shows three truncations of five different fungal Type II Transmembrane proteins. ‘SP’ refers to signal peptide; ‘TM’ refers to transmembrane; ‘Sc’ refers to Saccharomyces cerevisiae; ‘Pp’ refers to Pichia pastoris. The number indicates the amino acid sequence. KRE2 is a glycolipid 2-alpha-mannosyltransferase; MNN2 is alpha 1,2-mannosyltransferase; MNN1 is alpha 1,3-mannosyltransferase; MNN6 is mannosyltransferase, also referred to as KTR6. FIG. 7D shows the generation of engineered variants through the combination of three truncations of huFam20c (FIG. 7B) with fungal localization sequences (FIG. 7C).

FIGS. 8A-8D shows expression of human serine/threonine protein kinase Fam20c (huFam20c) with Pichia pastoris. Expression of Fam20c (M1-R584)_FLAG and four fragments (R32-R584_FLAG, R64-R854_FLAG, D93-R584 FLAG and Q289-R584_FLAG) using PichiaPink™ Strain 4 (Δade2, Δprb1, Δpep4). Strains were expressed in BMMY media, where methanol induces recombinant production, for 48 hours at 20° C. and 30° C. Supernatant samples were separated by denaturing electrophoresis, transferred to a nitrocellulose blot and probed with an HRP-conjugated Anti-DYDDDDK Mouse Monoclonal Antibody (“DYDDDDK” is disclosed as SEQ ID NO: 436). FIG. 8A shows a schematic of huFam20c structure and various truncated forms of huFam20c. The four truncations (R31, R64, D93 and Q289) are indicated. FIG. 8B shows that the native transmembrane domain of Fam20c (M1-R584) FLAG shows leaky secretion in yeast. If this sequence is removed (Fam20c (R32-R584)_FLAG) this effect is abolished. The leaky secretion is not a consequence of poor target expression, demonstrated by increased expression with a yeast signal peptide (α_Fam20c (R32-R584)_FLAG). FIG. 8C and FIG. 8D show that Fam20c (M1-R584) FLAG can be truncated in the N-terminus (α_Fam20c (R32-R584) FLAG, α_Fam20c (R64-R584) FLAG & α_Fam20c (D93-R584)_FLAG) to generate variants for localization but a minimal variant, α_Fam20c (Q289-R584) FLAG, shows poor expression and degradation. The image insert in FIG. 8D shows the blot following extended exposure.

FIGS. 9A-9C show in vivo phosphorylation with engineered human serine/threonine protein kinase Fam20c and corresponding unengineered control and unlocalized controls. Co-expression of FLAG_TRX_SPP1 (G158-N314)_HiBiT with engineered huFam20c and corresponding unengineered control and unlocalized controls in PichiaPink™ Strain 4 (Δade2, Δprb1, Δpep4) was tested. Strains were expressed in YPD media, where expression is constitutive, for 48 hours at 30° C. FIG. 9A shows supernatant samples separated by denaturing electrophoresis, transferred to a nitrocellulose blot and probed with an HRP-conjugated anti-DYDDDDK Mouse Monoclonal Antibody (“DYDDDDK” is disclosed as SEQ ID NO: 436). Migration of proteins in the gel is affected by phosphorylation. FIG. 9B shows quantitation of phosphorylation, where supernatant samples were acetone precipitated and intact isoforms identified with LC-MS. FIG. 9C shows the abundance of each phosphorylated isoform quantified by integrating the spectral peak associated with each. ‘ND’ refers to not detected.

FIG. 10 shows phosphorylation of non-recombinant and recombinant ovalbumin. Co-expression of FLAG_SERPINB14_HiBIT with un-engineered and engineered human serine/threonine protein kinase Fam20c (huFam20c) in PichiaPink™ Strain 4 (Δade2, Δprb1, Δpep4). Strains were expressed in YPD media, where expression is constitutive, for 72 hours at 30° C. Phosphorylation sites were detected in non-recombinant ovalbumin (Sigma-Aldrich A5503; amino acids underlined), detected in recombinant ovalbumin co-expressed with Fam20c (M1-R584) (triangle) and detected in recombinant ovalbumin co-expressed with ScMNN2 (M1-S36)_Fam20c (D93-R584)_FLAG (circle). Figure discloses SEQ ID NO: 439.

FIG. 11 shows phosphorylation of non-recombinant and recombinant αS-1 casein (I), αS-2 casein (II), and β-casein (III). Co-expression of caseins with un-engineered and engineered human serine/threonine protein kinase Fam20c (huFam20c) in Kluyveromyces lactis (Δku80, Δyps1). Strains were expressed in YPD media, where expression is constitutive, for 72 hours at 30° C. Phosphorylation sites were detected in non-recombinant caseins (amino acids underlined), detected in recombinant caseins co-expressed with Fam20c (M1-R584) (triangle) and detected in recombinant caseins co-expressed with ScMNN2 (M1-S36)_Fam20c (D93-R584) FLAG (circle). Figure discloses SEQ ID NOS 440-442, respectively, in order of appearance.

FIGS. 12A-12D show expression of recombinant Fam20c proteins in P. pastoris. FIG. 12A is a Western blot showing lemur Fam20c extracted from the extracellular portion of transgenic P. pastoris CBS7435. FIG. 12B is a Western blot showing sandgrouse Fam20c extracted from the extracellular portion of transgenic P. pastoris CBS7435. FIG. 12C is a Western blot showing lemur Fam20c extracted from the intracellular portion of transgenic P. pastoris CBS7435. FIG. 12D is a Western blot showing sandgrouse Fam20c extracted from the intracellular portion of transgenic P. pastoris CBS7435.

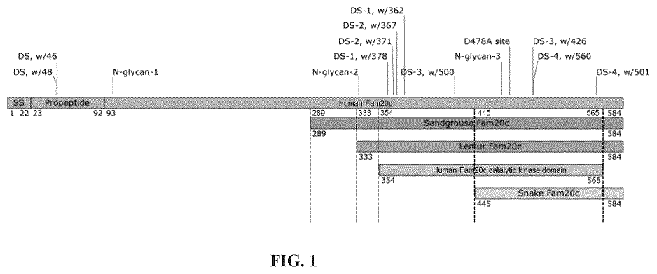

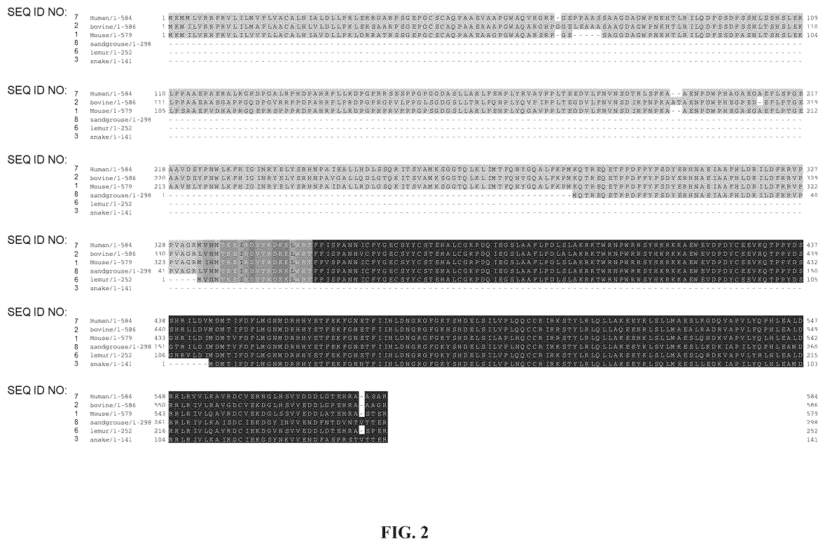

### DETAILED DESCRIPTION

### I. Introduction

Protein phosphorylation by protein kinases can contribute therapeutic and nutritional value to protein products. However, the challenge of expressing functional animal-derived protein kinases in non-animal cells presents a bottleneck that can preclude the production of such phosphorylated proteins in a cost-effective, humane, and environmentally conscious manner. Efficient production of native animal-derived phosphorylated proteins by non-animal cells may provide a cost-effective, humane, and environmentally friendly source for therapeutic and nutritional protein products. Described herein, in certain embodiments, are recombinant host cells (e.g., yeast) engineered to express kinases (e.g., serine/threonine kinases) and heterologous proteins (e.g., casein proteins). The kinases may be chimeric and/or heterologous in relationship to the recombinant host cell. The heterologous proteins may be of therapeutic and/or nutritional value and can comprise of one or more post-translational modification (e.g., phosphorylation) sites. The expressed heterologous protein expressed may be susceptible to phosphorylation by the expressed kinase.

The kinases may be serine/threonine kinases. In some embodiments, the kinases may be engineered with features (e.g., the inclusion of solubility enhancers) that improve the efficiency of phosphorylation on therapeutic and nutritional protein products. In some instances, the kinases may be less than 400 or less than 300 amino acids in length, and in some embodiments, may be less than 140 amino acids in length. Kinases with shorter amino acid lengths may increase the efficacy of phosphorylation on heterologous protein substrates in several ways. Reducing kinase length may decrease the number of proteolytic motifs and post-translational modification motifs, which may contribute to an increase in protein stability. This may enhance kinase phosphorylation efficacy by decreasing the risk of kinase proteolysis by enzymes endogenously expressed by the recombinant host. This may further enhance kinase phosphorylation efficacy by decreasing the risk of unproductive post-translational modifications made to the kinase by enzymes endogenously expressed by the recombinant host. Furthermore, kinases with shorter sequences may involve fewer amino acids for translation and may result in stronger kinase expression due to a reduced metabolic demand for protein production.

The kinases described herein may be targeted to a subcellular location of interest (e.g., the Golgi apparatus or the endoplasmic reticulum). In some embodiments, the kinases may be localized to a subcellular location of interest. In some embodiments, the kinases may be anchored to an intracellular membrane (e.g., nuclear membrane, and Golgi apparatus membrane, or an endoplasmic reticulum membrane). In some embodiments, the anchoring mechanism may be provided in part by an anchoring domain. In some embodiments, the anchoring domain may be covalently attached to the kinase. Targeted subcellular localization of kinases may increase phosphorylation efficacy on cognate substrates. Targeted localization of the kinase may increase the concentration of kinase at a particular subcellular location. Targeted localization of the kinase may increase half-life of the kinase. Targeted co-localization of the kinase and substrate may maximize the chances of kinase-substrate interactions for enzymatic catalysis. Targeted co-localization of the kinase and substrate may result in a higher efficiency of phosphorylation of target proteins during the secretion.

Described herein, in some embodiments, is a bioreactor system comprising a plurality of recombinant host cells, a reaction vessel (e.g., a shake flask), and media that may facilitate the expression of kinases and heterologous proteins. In some instances, the recombinant host cells and media may be disposed within the reaction vessel. Also described herein, is a method of manufacturing phosphorylated heterologous proteins that may have therapeutic and nutritional value. Such methods may use a cell population comprising a plurality of recombinant host cells and a bioreactor system. The resulting phosphorylated heterologous proteins described herein may contribute to the production of therapeutic, nutritional, or food products. Taken together, the embodiments described herein may provide a solution that enables efficient production of native animal-derived phosphorylated proteins by non-animal cells in a cost-effective, humane, and/or environmentally friendly manner.

The following definitions are set forth to facilitate explanation of the presently disclosed subject matter.

All technical and scientific terms used herein, unless otherwise defined below, are intended to have the same meaning as commonly understood by one of ordinary skill in the art. References to techniques employed herein are intended to refer to the techniques as commonly understood in the art, including variations on those techniques and/or substitutions of equivalent techniques that would be apparent to one of skill in the art.

Any ranges listed herein are intended to be inclusive of endpoints. For example, a range of 2-4 includes 2 and 4.

As used herein, the singular forms “a,” “an,” and “the” include plural references unless the content clearly dictates otherwise. For example, the term “a sample” includes a plurality of samples, including mixtures thereof.

The terms “about” or “approximately,” when immediately preceding a numerical value, refer to ±10% of the value provided. Furthermore, the phrases “less than about” a value or “greater than about” a value should be understood in view of the definition of the term “about” provided herein. Similarly, the term “about” when preceding a series of numerical values or a range of values (e.g., “about 10, 20, 30” or “about 10-30%”) refers, respectively to all values in the series, or the end points of the range.

Unless context clearly indicates otherwise, as used herein, the terms “including,” “includes,” “having,” “has,” “with,” or variants thereof are intended to be inclusive in a manner like the term “comprising.”

The term “percent (%) sequence identity,” with respect to a reference polypeptide sequence, is the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity. The term “percent (%) sequence identity,” with respect to a reference nucleic acid sequence, is the percentage of nucleic acid residues in a candidate sequence that are identical with the nucleic acid residues in the reference polynucleotide sequence, after aligning the sequences and introducing gaps, if necessary, to achieve the maximum percent sequence identity. Alignment for purposes of determining percent amino acid sequence or nucleic acid sequence identity can be achieved in various ways that are known, for instance, using publicly available computer software such as BLAST, BLAST-2, ALIGN or Megalign (DNASTAR) software. Appropriate parameters for aligning sequences are able to be determined, including algorithms needed to achieve maximal alignment over the full length of the sequences being compared. The % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) is calculated as follows: 100 times the fraction X/Y, where X is the number of amino acid residues scored as identical matches by the sequence alignment program in that program's alignment of A and B, and where Y is the total number of amino acid residues in B. It will be appreciated that where the length of amino acid sequence A is not equal to the length of amino acid sequence B, the % amino acid sequence identity of A to B will not equal the % amino acid sequence identity of B to A. Unless specifically stated otherwise, all % amino acid or nucleic acid sequence identity values used herein are obtained using the ALIGN-2 computer program. In assessing the percent sequence identity of a serine/threonine kinase to a specified sequence, all amino acids of the polypeptide chain that includes the serine/threonine kinase are aligned with the specified sequence, except that amino acids relating to a tag having its primary purpose to provide a functional handle for purification, identification, or to increase solubility are omitted. Such purification and/or identification tags may include FLAG or MYC. Such solubility enhancer tags include SUMO, TRX, MBP, MISTIC, NusA, FLAG-SUMO, FLAG-TRX, FLAG-MBP, FLAG-MISTIC, or FLAG-NusA tags.

The term “intact” when used in reference to a protein refers to a full-length protein.

The terms “heterologous” and “exogenous” refer to not being normally present in the context in which it is described. When used in reference to a protein expressed in a host cell, the term implies that the protein is not natively produced by the host cell.

The term “transformation” refers to a process by which a nucleic acid is introduced into a cell, either transiently or stably. Transformation may rely on any known method for the insertion of nucleic acid sequences into a prokaryotic or eukaryotic host cell, including Agrobacterium-mediated transformation protocols, viral infection, electroporation, heat shock, lipofection, polyethylene glycol treatment, micro-injection, and protoplasting.

The term “signal peptide” is used herein to refer to a peptide sequence that directs a protein to a specific cellular location or pathway. Signal peptides are often cleaved from a protein during translation or transport, and therefore may not be present in a mature protein.

The term “purifying” is used interchangeably with the term “isolating” and generally refers to the separation of a particular component from other components of the environment in which it was found or produced (e.g., membrane lipids, chromosomes, proteins). The terms allow but do not require that the purified or isolated component be separated from all other chemical components.

The terms “dairy protein” or “milk protein” refer to any protein, or fragment or variant thereof, that can be found in one or more mammalian milks. In some embodiments, the dairy proteins described herein are casein proteins, such as αs1-casein, αs2-casein, β-casein, and κ-casein.

The term “secretory protein” is used herein to refer to any protein, or fragment or variant thereof, that is secreted by the recombinant host cell into cell culture media.

All publications, patent applications, issued patents, and other documents referred to in this specification are herein incorporated by reference as if each individual publication, patent application, issued patent, or other document was specifically and individually indicated to be incorporated by reference in its entirety. Definitions that are contained in text incorporated by reference are excluded to the extent that they contradict definitions in this disclosure.

### I. Recombinant Host Cells

Recombinant host cells described herein, may be engineered to express (1) a protein with the capacity to catalyze protein post-translational modifications (e.g., kinases) and (2) a heterologous protein comprising one or more post-translational modification (e.g., phosphorylation) sites. Such proteins with the capacity to catalyze protein phosphorylation are referred to herein as kinases. The recombinant host cells herein may be derived from various taxonomic cell types (e.g., eukaryotic cells). In some embodiments, the recombinant host cell may be a fungal recombinant host cell (e.g., yeast), a bacterial recombinant host cell (e.g., E. coli), an algal recombinant host cell, or a plant recombinant host cell. In some embodiments, a fungal recombinant host cell may be a yeast cell wherein the yeast cell is Kluyveromyces lactis or Pichia pastoris. In some embodiments, a fungal recombinant host cell may be filamentous fungus, such as Aspergillus or Thermothelomyces heterothallica. In some instances, a fungal recombinant host cell may be Candida, Fusarium, Hansenula, Penicillium, Saccharomyces, Tetrahymena, Thermothelomyces, Trichoderma, Yarrowia, or Zygosaccharomyces.

In some embodiments, a fungal recombinant host cell can comprise a heterologous kinase, wherein the heterologous kinase is a Fam20c member. In some embodiments, the heterologous kinase can be a human Fam20c member. In some embodiments, the Fam20c member can be a truncated Fam20c member.

A fungal recombinant host cell can comprise any one of the non-naturally occurring polypeptides described herein and can include any non-naturally occurring polypeptides comprising a Fam20c protein.

### Expressed Kinases

Recombinant host cells described herein may be engineered to express a kinase (e.g., a serine/threonine kinase) with the capacity to phosphorylate protein substrates of interest. The kinases described herein may be heterologous with respect to the recombinant host cell, may be derived in part from serine/threonine kinases, and may have multiple features. In some embodiments, the expressed kinase may be a reptilian serine/threonine kinase, wherein the reptilian serine/threonine kinase may be a snake serine/threonine kinase. In some embodiments, the expressed kinase may have at least 80%, 90%, 95%, or 99% sequence identity to SEQ ID NO 3. In some embodiments, the expressed kinase may have at least 80% sequence identity to SEQ ID NO: 3 but does not include a sequence of greater than 20 amino acids in length having 90%, 95%, or 100% sequence identity to SEQ ID NO: 9. In some embodiments, the expressed kinase may be less than 140, 200, or 300 amino acids in length and may have at least 80%, 90%, 95%, or 99% sequence identity to SEQ ID NO: 3. In some embodiments, the expressed kinase may be less than 140, 200, or 300 amino acids in length and may have at least 80% sequence identity to SEQ ID NO: 3 but does not include a sequence of greater than 20 amino acids in length having 90% sequence identity to SEQ ID NO: 9. In some instances, the expressed kinase is a snake Fam20c kinase or a biologically active portion thereof.

In some embodiments, the expressed kinase may be a primatial serine/threonine kinase, wherein the primatial kinase may be a lemur serine/threonine kinase. In some embodiments, the expressed kinase may have at least 75%, 80%, 90%, 95%, or 100% sequence identity to SEQ ID NO: 6.

In some embodiments, the expressed kinase may be an avian serine/threonine kinase, wherein the avian serine/threonine kinase may be a sandgrouse serine/threonine kinase. In some embodiments, the expressed kinase may have at least 75%, 80%, 90%, 95%, or 100% sequence identity to SEQ ID NO: 8.

In some embodiments, the expressed kinase may be a human serine/threonine kinase. In some embodiments, the human serine/threonine kinase may be a Fam20c kinase. In some embodiments, the expressed kinase may have at least 75%, 80%, 90%, 95%, or 100% sequence identity to SEQ ID NO: 7.

In some embodiments, the human serine/threonine kinase may be a Fam20c kinase having an amino acid sequence of SEQ ID NO: 145. In some embodiments, the expressed kinase may have at least 75%, 80%, 90%, 95%, or 100% sequence identity to SEQ ID NO: 145. In some embodiments, the human serine/threonine kinase may be a Fam20c kinase comprising a truncation at an N-terminal end of up to 1 amino acid, up to 2 amino acids, up to 3 amino acids, up to 4 amino acids, up to 5 amino acids, up to 6 amino acids, up to 7 amino acids, up to 8 amino acids, up to 9 amino acids, up to 10 amino acids, up to 11 amino acids, up to 12 amino acids, up to 13 amino acids, up to 14 amino acids, up to 15 amino acids, up to 16 amino acids, up to 17 amino acids, up to 18 amino acids, up to 19 amino acids, up to 20 amino acids, up to 21 amino acids, up to 22 amino acids, up to 23 amino acids, up to 24 amino acids, up to 25 amino acids, up to 26 amino acids, up to 27 amino acids, up to 28 amino acids, up to 29 amino acids, up to 30 amino acids, up to 31 amino acids, up to 32 amino acids, up to 33 amino acids, up to 34 amino acids, up to 35 amino acids, up to 36 amino acids, up to 37 amino acids, up to 38 amino acids, up to 39 amino acids, up to 40 amino acids, up to 41 amino acids, up to 42 amino acids, up to 43 amino acids, up to 44 amino acids, up to 45 amino acids, up to 46 amino acids, up to 47 amino acids, up to 48 amino acids, up to 49 amino acids, up to 50 amino acids, up to 51 amino acids, up to 52 amino acids, up to 53 amino acids, up to 54 amino acids, up to 55 amino acids, up to 56 amino acids, up to 57 amino acids, up to 58 amino acids, up to 59 amino acids, up to 60 amino acids, up to 61 amino acids, up to 62 amino acids, up to 63 amino acids, up to 64 amino acids, up to 65 amino acids, up to 66 amino acids, up to 67 amino acids, up to 68 amino acids, up to 69 amino acids, up to 70 amino acids, up to 71 amino acids, up to 72 amino acids, up to 73 amino acids, up to 74 amino acids, up to 75 amino acids, up to 76 amino acids, up to 77 amino acids, up to 78 amino acids, up to 79 amino acids, up to 80 amino acids, up to 81 amino acids, up to 82 amino acids, up to 83 amino acids, up to 84 amino acids, up to 85 amino acids, up to 86 amino acids, up to 87 amino acids, up to 88 amino acids, up to 89 amino acids, up to 90 amino acids, up to 91 amino acids, up to 92 amino acids, or up to 93 amino acids. In some embodiments, the human serine/threonine kinase may be a Fam20c kinase having an amino acid sequence that is 584 amino acids in length.

(SEQ ID NO: 145)
MKMMLVRRFRVLILMVFLVACALHIALDLLPRLERRGARPSGEPGCS
CAQPAAEVAAPGWAQVRGRPGEPPAASSAAGDAGWPNKHTLRILQDF
SSDPSSNLSSHSLEKLPPAAEPAERALRGRDPGALRPHDPAHRPLLR
DPGPRRSESPPGPGGDASLLARLFEHPLYRVAVPPLTEEDVLFNVNS
DTRLSPKAAENPDWPHAGAEGAEFLSPGEAAVDSYPNWLKFHIGINR
YELYSRHNPAIEALLHDLSSQRITSVAMKSGGTQLKLIMTFQNYGQA
LFKPMKQTREQETPPDFFYFSDYERHNAEIAAFHLDRILDFRRVPPV
AGRMVNMTKEIRDVTRDKKLWRTFFISPANNICFYGECSYYCSTEHA
LCGKPDQIEGSLAAFLPDLSLAKRKTWRNPWRRSYHKRKKAEWEVDP
DYCEEVKQTPPYDSSHRILDVMDMTIFDFLMGNMDRHHYETFEKFGN
ETFIIHLDNGRGFGKYSHDELSILVPLQQCCRIRKSTYLRLQLLAKE
EYKLSLLMAESLRGDQVAPVLYQPHLEALDRRLRVVLKAVRDCVERN
GLHSVVDDDLDTEHRAASAR.

In some embodiments, the human serine/threonine kinase may be a Fam20c kinase further comprising a FLAG tag having an amino acid sequence of SEQ ID NO: 146. In some embodiments, the expressed kinase may further comprise a FLAG tag and may have at least 75%, 80%, 90%, 95%, or 100% sequence identity to the sequence set forth in SEQ ID NO: 146.

(SEQ ID NO: 146)
MKMMLVRRFRVLILMVFLVACALHIALDLLPRLERRGARPSGEPGCS
CAQPAAEVAAPGWAQVRGRPGEPPAASSAAGDAGWPNKHTLRILQDF
SSDPSSNLSSHSLEKLPPAAEPAERALRGRDPGALRPHDPAHRPLLR
DPGPRRSESPPGPGGDASLLARLFEHPLYRVAVPPLTEEDVLFNVNS
DTRLSPKAAENPDWPHAGAEGAEFLSPGEAAVDSYPNWLKFHIGINR
YELYSRHNPAIEALLHDLSSQRITSVAMKSGGTQLKLIMTFQNYGQA
LFKPMKQTREQETPPDFFYFSDYERHNAEIAAFHLDRILDFRRVPPV
AGRMVNMTKEIRDVTRDKKLWRTFFISPANNICFYGECSYYCSTEHA
LCGKPDQIEGSLAAFLPDLSLAKRKTWRNPWRRSYHKRKKAEWEVDP
DYCEEVKQTPPYDSSHRILDVMDMTIFDFLMGNMDRHHYETFEKFGN
ETFIIHLDNGRGFGKYSHDELSILVPLQQCCRIRKSTYLRLQLLAKE
EYKLSLLMAESLRGDQVAPVLYQPHLEALDRRLRVVLKAVRDCVERN
GLHSVVDDDLDTEHRAASARDYKDDDDK.

In some embodiments, the human serine/threonine kinase may be a Fam20c kinase further comprising a MYC tag having an amino acid sequence of SEQ ID NO: 147. In some embodiments, the expressed kinase may further comprise a MYC tag and may have at least 75%, 80%, 90%, 95%, or 100% sequence identity to the sequence set forth in SEQ ID NO: 147.

(SEQ ID NO: 147)
MKMMLVRRFRVLILMVFLVACALHIALDLLPRLERRGARPSGEPGCS
CAQPAAEVAAPGWAQVRGRPGEPPAASSAAGDAGWPNKHTLRILQDF
SSDPSSNLSSHSLEKLPPAAEPAERALRGRDPGALRPHDPAHRPLLR
DPGPRRSESPPGPGGDASLLARLFEHPLYRVAVPPLTEEDVLFNVNS
DTRLSPKAAENPDWPHAGAEGAEFLSPGEAAVDSYPNWLKFHIGINR
YELYSRHNPAIEALLHDLSSQRITSVAMKSGGTQLKLIMTFQNYGQA
LFKPMKQTREQETPPDFFYFSDYERHNAEIAAFHLDRILDFRRVPPV
AGRMVNMTKEIRDVTRDKKLWRTFFISPANNICFYGECSYYCSTEHA
LCGKPDQIEGSLAAFLPDLSLAKRKTWRNPWRRSYHKRKKAEWEVDP
DYCEEVKQTPPYDSSHRILDVMDMTIFDFLMGNMDRHHYETFEKFGN
ETFIIHLDNGRGFGKYSHDELSILVPLQQCCRIRKSTYLRLQLLAKE
EYKLSLLMAESLRGDQVAPVLYQPHLEALDRRLRVVLKAVRDCVERN
GLHSVVDDDLDTEHRAASAREQKLISEEDL.

In some embodiments, the human serine/threonine kinase may be a Fam20c kinase having an amino acid sequence of SEQ ID NO: 148. In some embodiments, the expressed kinase may have at least 75%, 80%, 90%, 95%, or 100% sequence identity according to the amino acid sequence set forth in SEQ ID NO: 148.

(SEQ ID NO: 148)
KMMLVRRFRVLILMVFLVACALHIALDLLPRLERRGARPSGEPGCSC
AQPAAEVAAPGWAQVRGRPGEPPAASSAAGDAGWPNKHTLRILQDFS
SDPSSNLSSHSLEKLPPAAEPAERALRGRDPGALRPHDPAHRPLLRD
PGPRRSESPPGPGGDASLLARLFEHPLYRVAVPPLTEEDVLFNVNSD
TRLSPKAAENPDWPHAGAEGAEFLSPGEAAVDSYPNWLKFHIGINRY
ELYSRHNPAIEALLHDLSSQRITSVAMKSGGTQLKLIMTFQNYGQAL
FKPMKQTREQETPPDFFYFSDYERHNAEIAAFHLDRILDFRRVPPVA
GRMVNMTKEIRDVTRDKKLWRTFFISPANNICFYGECSYYCSTEHAL
CGKPDQIEGSLAAFLPDLSLAKRKTWRNPWRRSYHKRKKAEWEVDPD
YCEEVKQTPPYDSSHRILDVMDMTIFDFLMGNMDRHHYETFEKFGNE
TFIIHLDNGRGFGKYSHDELSILVPLQQCCRIRKSTYLRLQLLAKEE
YKLSLLMAESLRGDQVAPVLYQPHLEALDRRLRVVLKAVRDCVERNG
LHSVVDDDLDTEHRAASAR.

In some embodiments, the human serine/threonine kinase may be a Fam20c kinase having an endogenous secretion signal according to the amino acid sequence set forth in SEQ ID NO: 149.

(SEQ ID NO: 149)
MKMMLVRRFRVLILMVFLVACA

In some embodiments, the human serine/threonine kinase may be a Fam20c kinase having a pro-peptide according to the amino acid sequence set forth in of SEQ ID NO: 150.

(SEQ ID NO: 150)
LHIALDLLPRLERRGARPSGEPGCSCAQPAAEVAAPGWAQVRGRPG
EPPAASSAAGDAGWPNKHTLRILQ.

In some embodiments, the human serine/threonine kinase may be a Fam20c kinase having a transmembrane domain according to the amino acid sequence set forth in of SEQ ID NO: 151.

(SEQ ID NO: 151)
VLILMVFLVACALHIALDLLP.

In some embodiments, the human serine/threonine kinase may be a Fam20c kinase having a stem region according to the amino acid sequence set forth in of SEQ ID NO: 152.

(SEQ ID NO: 152)
DFSSDPSSNLSSHSLEKLPPAAEPAERALRGRDPGALRPHDPAHRP
LLRDPGPRRSESPPGPGGDASLLARLFEHPLYRVAVPPLTEEDVLF
NVNSDTRLSPKAAENPDWPHAGAEGAEFLSPGEAAVDSYPNWLKFH
IGINRYELYSRHNPAIEALLHDLSSQRITSVAMKSGGTQLKLIMTF
QNYGQALFKPMKQTREQETPPDFFYFSDYERHNAEIAAFHLDRILD
FRRVPPVAGRMVNMTKEIRDVTRDKKLWRTF.

In some embodiments, the human serine/threonine kinase may be a Fam20c kinase having a catalytic kinase domain according to the amino acid sequence set forth in of SEQ ID NO: 153.

(SEQ ID NO: 153)
FISPANNICFYGECSYYCSTEHALCGKPDQIEGSLAAFLPDLSLAK
RKTWRNPWRRSYHKRKKAEWEVDPDYCEEVKQTPPYDSSHRILDVM
DMTIFDFLMGNMDRHHYETFEKFGNETFIIHLDNGRGFGKYSHDEL
SILVPLQQCCRIRKSTYLRLQLLAKEEYKLSLLMAESLRGDQVAPV
LYQPHLEALDRRLRVVLKAVRDCVERNG.

In some embodiments, the expressed serine/threonine kinase may be a Fam20c kinase having at least 75%, 80%, 90%, 95%, or 100% sequence identity according to an amino acid sequence of any one of SEQ ID NOs: 93-144 and 154. In some embodiments, the expressed serine/threonine kinase may be encoded by a nucleic acid sequence having at least 75%, 80%, 90%, 95%, or 100% sequence identity according to a nucleic acid sequence of any one of SEQ ID NOs: 185-238 and 302-303. In some embodiments, the expressed serine/threonine kinase may be encoded by a vector having at least 75%, 80%, 90%, 95%, or 100% sequence identity according to a nucleic acid sequence of any one of SEQ ID NOs: 241-295 and 309-310.

TABLE 1 provides exemplary full-length Fam20c and Fam20c fragment sequences.

TABLE 1
Exemplary full-length Fam20c
and Fam20c Fragment Polypeptide Sequences

| SEQ ID | Sequence | |
|---|---|---|
| NO: | Name | Sequence |
| 1 | Mouse | MKMILVRRFRVLILVVFLLACALHIAVDLLPKLDRRATRS |
| | Fam20c | SGEPGCSCAQPAAEAAGPGWAQARSRPGESAGGDAGWP |
| | | NKHTLRILQDFSSDPASNLTSHSLEKLPSAAEPVDHAPRG |
| | | QEPRSPPPRDPAHRPLLRDPGPRPRVPPPGPSGDGSLLAK |
| | | LFEHPLYQGAVPPLTEDDVLFNVNSDIRFNPKAAENPDWP |
| | | HEGAEGAEFLPTGEAAVNLYPNWLKFHIGINRYELYSRH |
| | | NPAIDALLRDLGSQKITSVAMKSGGTQLKLIMTFQNYGQ |
| | | ALFKPMKQTREQETPPDFFYFSDYERHNAEIAAFHLDRIL |
| | | DFRRVPPVAGRMINMTKEIRDVTRDKKLWRTFFVSPANN |
| | | ICFYGECSYYCSTEHALCGRPDQIEGSLAAFLPDLSLAKR |
| | | KTWRNPWRRSYHKRKKAEWEVDPDYCEEVKQTPPYDS |
| | | GHRILDIMDMTVFDFLMGNMDRHHYETFEKFGNETFIIH |
| | | LDNGRGFGKYSHDELSILAPLHQCCRIRRSTYLRLQLLAK |
| | | EEHKLSLLMAESLQHDKVAPVLYQLHLEALDRRLRIVLQ |
| | | AVRDCVEKDGLSSVVEDDLATEHRASTER |
| 2 | Bovine | MKMILVRRFRVLILMAFLAACALHLVLDLLPKLERSAAR |
| | Fam20c | PSGEPGCSCAQPAAEAAAPGWAQARGHPGGELEAAASA |
| | | AGDAGWPNKHTLRILQDFSSDPSSNLTSHSLEKLPPAAEA |
| | | AEGAPPGQDPGVRRPPDPAHRPLPRDPGPRGPVLPPGLS |
| | | GDGSLLTRLFQHPLYQVPIPPLTEGDVLFNVNSDIRFNPK |
| | | AATAENPDWPHEGPEDEFLPTGEAAVDSYPNWLKFHIGI |
| | | NRYELYSRHNPAVGALLQDLGTQKITSVAMKSGGTQLKL |
| | | IMTFQNYGQALFKPMKQTREQETPPDFFYFSDYERHNAE |
| | | IAAFHLDRILDFRRVPPVAGRLVNMTKEIRDVTRDKKLW |
| | | RTFFISPANNVCFYGECSYYCSTEHALCGKPDQIEGSLAA |
| | | FLPDLALAKRKTWRNPWRRSYHKRKKAEWEVDPDYCE |
| | | EVRQTPPYDSSHRLLDVMDMTIFDFLMGNMDRHHYETF |
| | | EKFGNETFIIHLDNGRGFGKHSHDELSILVPLQQCCRIRRS |
| | | TYLRLQLLAQEEHRLSLLMAEALRADRVAPVLFQPHLEA |
| | | LDRRLRIVLRAVGDCVEKDGLHSVVEDDLGPEHRAAAGR |
| 3 | Snake | MDMTIFDFLMGNMDRHHYETFEKFGNNTFIIHLDNGRGF |
| | Fam20c | GKYSHDELSILVPLNQCCRIRKSTYLRLQLLAKEEYKLSQ |
| | | LMEESLLEDKIAPILYQLHLEAMDRRLRIVLKAIRDCIEK |
| | | GSYNKVVENDFASPRSTVTTER |
| 6 | Lemur | MVNMTKEIRDVTRDKKLWRTFFISPANNICFYGECSYYC |
| | Fam20c | STEHALCGKPDQIEGSLAAFLPDLSLAKRKTWRNPWRRS |
| | | YHKRKKAEWEVDPDYCEEVKQTPPYDSGHRVLDIMDMT |
| | | IFDFLMGNMDRHHYETFEKFGNETFIIHLDNGRGFGKYS |
| | | HDELSILVPLQQCCRIRKSTYLRLQLLAKEEYKLSLLMAE |
| | | SLQRDKVAPVLYRLHLEALDRRLRIVLQAVRDCIEKDGV |
| | | HSVVEDDLDTEHRASPER |
| 7 | Human | MKMMLVRRFRVLILMVFLVACALHIALDLLPRLERRGA |
| | Fam20c | RPSGEPGCSCAQPAAEVAAPGWAQVRGRPGEPPAASSAA |
| | | GDAGWPNKHTLRILQDFSSDPSSNLSSHSLEKLPPAAEPA |
| | | ERALRGRDPGALRPHDPAHRPLLRDPGPRRSESPPGPGG |
| | | DASLLARLFEHPLYRVAVPPLTEEDVLFNVNSDTRLSPKA |
| | | AENPDWPHAGAEGAEFLSPGEAAVDSYPNWLKFHIGINR |
| | | YELYSRHNPAIEALLHDLSSQRITSVAMKSGGTQLKLIMT |
| | | FQNYGQALFKPMKQTREQETPPDFFYFSDYERHNAEIAA |
| | | FHLDRILDFRRVPPVAGRMVNMTKEIRDVTRDKKLWRT |
| | | FFISPANNICFYGECSYYCSTEHALCGKPDQIEGSLAAFLP |
| | | DLSLAKRKTWRNPWRRSYHKRKKAEWEVDPDYCEEVK |
| | | QTPPYDSSHRILDVMDMTIFDFLMGNMDRHHYETFEKFG |
| | | NETFIIHLDNGRGFGKYSHDELSILVPLQQCCRIRKSTYLR |
| | | LQLLAKEEYKLSLLMAESLRGDQVAPVLYQPHLEALDRR |
| | | LRVVLKAVRDCVERNGLHSVVDDDLDTEHRAASAR |
| 8 | Sandgrouse | MQTREQETPPDFFYFSDYERHNAEIAAFHLDRILDFRRVP |
| | Fam20c | PVAGRLVNMTREIRDVTRDKKLWRTFFISPANNICFYGEC |
| | | SYYCSTEHALCGKPDQIEGSLAAFLPDLSLAKRKTWRNP |
| | | WRRSYHKRKKAEWEVDPDYCEEVKQTPPYDSGTRILDI |
| | | MDMTVFDFLMGNMDRHHYETFEKFGNETFIIHLDNGRG |
| | | FGKYSHDELSILVPLNQCCRIRKSTYLRLQLLAKEEYKLS |
| | | VLMKESLLKDKIAPILYQPHLEAMDRRLRIVLKAISDCIE |
| | | KDGYINVVENDFNTDVNTVTTER |
| 9 | N-terminal | KMMLVRRFRVLILMVFLVACALHIALDLLPRLERRGARP |
| | portion of | SGEPGCSCAQPAAEVAAPGWAQVRGRPGEPPAASSAAGD |
| | human | AGWPNKHTLRILQDFSSDPSSNLSSHSLEKLPPAAEPAER |
| | Fam20c | ALRGRDPGALRPHDPAHRPLLRDPGPRRSESPPGPGGDA |
| | | SLLARLFEHPLYRVAVPPLTEEDVLFNVNSDTRLSPKAAE |
| | | NPDWPHAGAEGAEFLSPGEAAVDSYPNWLKFHIGINRYE |
| | | LYSRHNPAIEALLHDLSSQRITSVAMKSGGTQLKLIMTFQ |
| | | NYGQALFKPMK |

In some embodiments, the expressed kinase may further comprise a purification and/or identification tag. The purification and/or identification tag may be a FLAG tag that comprises SEQ ID NO: 10. The purification and/or identification tag may be a MYC tag that comprises SEQ ID NO: 311. In some embodiments, the expressed kinase may further comprise a solubility enhancer tag, wherein the solubility enhancer tag may be a SUMO tag that comprises SEQ ID NO: 11, a TRX tag that comprises SEQ ID NO: 12, a MBP tag that comprises SEQ ID NO: 13, a MISTIC tag that comprises SEQ ID NO: 14, a NusA tag that comprises SEQ ID NO: 15, a FLAG-SUMO tag that comprises SEQ ID NO: 16, a FLAG-TRX tag that comprises SEQ ID NO: 17, a FLAG-MBP tag that comprises SEQ ID NO: 18, a FLAG-MISTIC tag that comprises SEQ ID NO: 19, or a FLAG-NusA tag that comprises SEQ ID NO: 20. In some embodiments, an N-terminal methionine may be added to the purification and/or identification and solubility enhancer tags in SEQ ID NOs: 10-20.

TABLE 2 provides exemplary purification, identification, and solubility enhancer tag sequences.

TABLE 2
Exemplary Purification, Identification, and Solubility Enhancer
Tag Polypeptide Sequences

| SEQ ID | Sequence | |
|---|---|---|
| NO: | Name | Sequence |
| 10 | FLAG | DYKDDDDK |
| 11 | SUMO | GSLQEEKPKEGVKTENDHINLKVAGQDGSVVQFKIKRHT |
| | | PLSKLMKAYCERQGLSMRQIRFREDGQPINETDTPAQLE |
| | | MEDEDTIDVFQQQTGGEFDP |
| 12 | TRX | SDKIIHLTDDSFDTDVLKADGAILVDFWAEWCGPCKMIA |
| | | PILDEIADEYQGKLTVAKLNIDQNPGTAPKYGIRGIPTLLL |
| | | FKNGEVAATKVGALSKGQLKEFLDANLA |
| 13 | MBP | KIKTGARILALSALTTMMFSASALAKIEEGKLVIWINGD |
| | | KGYNGLAEVGKKFEKDTGIKVTVEHPDKLEEKFPQVAAT |
| | | GDGPDIIFWAHDRFGGYAQSGLLAEITPDKAFQDKLYPFT |
| | | WDAVRYNGKLIAYPIAVEALSLIYNKDLLPNPPKTWEEIP |
| | | ALDKELKAKGKSALMENLQEPYFTWPLIAADGGYAFKY |
| | | ENGKYDIKDVGVDNAGAKAGLTFLVDLIKNKHMNADTD |
| | | YSIAEAAFNKGETAMTINGPWAWSNIDTSKVNYGVTVLP |
| | | TFKGQPSKPFVGVLSAGINAASPNKELAKEFLENYLLTDE |
| | | GLEAVNKDKPLGAVALKSYEEELAKDPRIAATMENAQK |
| | | GEIMPNIPQMSAFWYAVRTAVINAASGRQTVDEALKDAQ |
| | | TRITK |
| 14 | MISTIC | FCTFFEKHHRKWDILLEKSTGVMEAMKVTSEEKEQLSTA |
| | | IDRMNEGLDAFIQLYNESEIDEPLIQLDDDTAELMKQARD |
| | | MYGQEKLNEKLNTIIKQILSISVSEEGEKE |
| 15 | NusA | NKEILAVVEAVSNEKALPREKIFEALESALATATKKKYEQ |
| | | EIDVRVQIDRKSGDFDTFRRWLVVDEVTQPTKEITLEAAR |
| | | YEDESLNLGDYVEDQIESVTFDRITTQTAKQVIVQKVREA |
| | | ERAMVVDQFREHEGEIITGVVKKVNRDNISLDLGNNAEA |
| | | VILREDMLPRENFRPGDRVRGVLYSVRPEARGAQLFVTR |
| | | SKPEMLIELFRIEVPEIGEEVIEIKAAARDPGSRAKIAVKT |
| | | NDKRIDPVGACVGMRGARVQAVSTELGGERIDIVLWDDN |
| | | PAQFVINAMAPADVASIVVDEDKHTMDIAVEAGNLAQAI |
| | | GRNGQNVRLASQLSGWELNVMTVDDLQAKHQAEAHAAI |
| | | DTFTKYLDIDEDFATVLVEEGFSTLEELAYVPMKELLEIE |
| | | GLDEPTVEALRERAKNALATIAQAQEESLGDNKPADDLL |
| | | NLEGVDRDLAFKLAARGVCTLEDLAEQGIDDLADIEGLT |
| | | DEKAGALIMAARNICWFGDEA |
| 16 | FLAG-SUMO | GDYKDDDDKGSLQEEKPKEGVKTENDHINLKVAGQDGS |
| | | VVQFKIKRHTPLSKLMKAYCERQGLSMRQIRFRFDGQPI |
| | | NETDTPAQLEMEDEDTIDVFQQQTGGEFDP |
| 17 | FLAG-TRX | GDYKDDDDKMSDKIIHLTDDSFDTDVLKADGAILVDFWA |
| | | EWCGPCKMIAPILDEIADEYQGKLTVAKLNIDQNPGTAP |
| | | KYGIRGIPTLLLFKNGEVAATKVGALSKGQLKEFLDANL |
| | | A |
| 18 | FLAG-MBP | GDYKDDDDKMKIKTGARILALSALTTMMFSASALAKIEE |
| | | GKLVIWINGDKGYNGLAEVGKKFEKDTGIKVTVEHPDK |
| | | LEEKFPQVAATGDGPDIIFWAHDRFGGYAQSGLLAEITPD |
| | | KAFQDKLYPFTWDAVRYNGKLIAYPIAVEALSLIYNKDLL |
| | | PNPPKTWEEIPALDKELKAKGKSALMENLQEPYFTWPLI |
| | | AADGGYAFKYENGKYDIKDVGVDNAGAKAGLTFLVDLI |
| | | KNKHMNADTDYSIAEAAFNKGETAMTINGPWAWSNIDTS |
| | | KVNYGVTVLPTFKGQPSKPFVGVLSAGINAASPNKELAK |
| | | EFLENYLLTDEGLEAVNKDKPLGAVALKSYEEELAKDPR |
| | | IAATMENAQKGEIMPNIPQMSAFWYAVRTAVINAASGRQ |
| | | TVDEALKDAQTRITK |
| 19 | FLAG- | GDYKDDDDKMFCTFFEKHHRKWDILLEKSTGVMEAMK |
| | MISTIC | VTSEEKEQLSTAIDRMNEGLDAFIQLYNESEIDEPLIQLDD |
| | | DTAELMKQARDMYGQEKLNEKLNTIIKQILSISVSEEGEK |
| | | E |
| 20 | FLAG-NusA | GDYKDDDDKMNKEILAVVEAVSNEKALPREKIFEALESA |
| | | LATATKKKYEQEIDVRVQIDRKSGDEDTFRRWLVVDEVT |
| | | QPTKEITLEAARYEDESLNLGDYVEDQIESVTFDRITTQTA |
| | | KQVIVQKVREAERAMVVDQFREHEGEIITGVVKKVNRDN |
| | | ISLDLGNNAEAVILREDMLPRENFRPGDRVRGVLYSVRPE |
| | | ARGAQLFVTRSKPEMLIELFRIEVPEIGEEVIEIKAAARDP |
| | | GSRAKIAVKINDKRIDPVGACVGMRGARVQAVSTELGG |
| | | ERIDIVLWDDNPAQFVINAMAPADVASIVVDEDKHTMDIA |
| | | VEAGNLAQAIGRNGQNVRLASQLSGWELNVMTVDDLQA |
| | | KHQAEAHAAIDTFTKYLDIDEDFATVLVEEGFSTLEELAY |
| | | VPMKELLEIEGLDEPTVEALRERAKNALATIAQAQEESLG |
| | | DNKPADDLLNLEGVDRDLAFKLAARGVCTLEDLAEQGID |
| | | DLADIEGLTDEKAGALIMAARNICWFGDEA |
| 311 | MYC | EQKLISEEDL |

### Targeted Subcellular Localization

Recombinant host cells described herein may be engineered to express a kinase (e.g., a serine/threonine kinase) that may be targeted to a subcellular location of interest (e.g., the Golgi apparatus or ER). In some instances, the kinase may be localized to a subcellular location of interest. In some instances, the kinase may be anchored to an intracellular membrane (e.g., cell membrane). In some instances, the anchoring mechanism may be provided in part by an anchoring domain. In some embodiments, the anchoring domain may be covalently attached to the kinase. The intracellular membrane described herein may be a Golgi apparatus membrane, an ER membrane, or a mitochondrial membrane. In some embodiments, the kinase may be anchored such that a kinase domain is positioned on the luminal side of the Golgi apparatus membrane. In some embodiments, the kinase may be anchored to an intracellular membrane via a polypeptide. In some embodiments, the polypeptide may be covalently attached to the kinase. In some embodiments, the polypeptide may be attached to the N-terminus or the C-terminus of the kinase.

In some embodiments, the polypeptide comprises a Type I or a Type II transmembrane protein. In some embodiments, the polypeptide comprises a Type II transmembrane protein. In some embodiments, the Type II transmembrane protein may have at least 75%, 80%, 90%, 95%, or 100% sequence identity to an amino acid sequence of any one of SEQ ID NOs: 155-159.

In some embodiments, the polypeptide comprises a fragment of a Type I or a Type II transmembrane protein. In some embodiments, the polypeptide comprises a fragment of a Type II transmembrane protein. In some embodiments, the polypeptide comprises a Type I, a Type II, or a multi-pass transmembrane domain. In some embodiments, the polypeptide comprises a Type II transmembrane domain. In some embodiments, the Type II transmembrane domain may have at least 75%, 80%, 90%, 95%, or 100% sequence identity to an amino acid sequence of any one of SEQ ID NOs: 181-184. In some embodiments, the polypeptide comprises a fragment of a Type II transmembrane protein. In some embodiments, the Type II transmembrane protein may have at least 75%, 80%, 90%, 95%, or 100% sequence identity to an amino acid sequence of any one of SEQ ID NOs: 160-174. In some embodiments, the polypeptide comprises a Type II transmembrane domain.

In some embodiments, the fragment of a Type II transmembrane protein comprises the Type II transmembrane protein comprising a truncation at a C-terminal end. In some embodiments, the fragment of a Type II transmembrane protein comprises the Type II transmembrane protein comprising a truncation of up to 50 amino acids, up to 100 amino acids, up to 150 amino acids, up to 200 amino acids, up to 250 amino acids, up to 300 amino acids, up to 350 amino acids, up to 400 amino acids, up to 450 amino acids, up to 500 amino acids, up to 550 amino acids, up to 600 amino acids, up to 650 amino acids, up to 700 amino acids, or up to 750 amino acids.

In some embodiments, the polypeptide comprises a Golgi retention sequence. In some embodiments, the polypeptide comprises an amino acid sequence of any one of SEQ ID NOs: 21-22. In some embodiments, the amino acid sequence of any one of SEQ ID NOs: 21-22 is at the C-terminus of the polypeptide.

In some embodiments, the polypeptide comprises an amino acid sequence having at least 75%, 80%, 85%, 90%, 95% sequence identity to any one of SEQ ID NOs: 23-85. In some embodiments, the polypeptide comprises an amino acid sequence of any one of SEQ ID NOs: 23-85. In some embodiments, the polypeptide comprises an amino acid sequence of SEQ ID NO: 71.

In some instances, the kinase may be anchored to an intracellular membrane via a lipid. In some instances, the lipid may be covalently attached to the kinase. In some instances, the kinase and lipid may form a prenylated protein. In some instances, the lipid attached to the kinase may comprise a fatty acyl group. In some instances, the lipid and kinase form a glycosylphosphatidylinositol-linked protein.

TABLE 3
TABLE 3 provides exemplary Golgi
apparatus retrieval sequences.
Exemplary Golgi Apparatus
Retrieval Sequences

| SEQ ID | Sequence | |
|---|---|---|
| NO: | Name | Sequence |
| 21 | HDEL | HDEL |
| 22 | KDEL | KDEL |

TABLE 4 provides exemplary Golgi apparatus and endoplasmic reticulum retention sequences.

TABLE 4
Exemplary Golgi Apparatus and Endoplasmic
Reticulum Retention Sequences

| SEQ ID | | | |
|---|---|---|---|
| NO: | Location | Gene ID | Sequence |
| 23 | ER | ScGLS1-s | MLISKSKMFKTFWILTSIVLLASATVDISKLQE |
| | | | F |
| 24 | ER | ScGLS1-m | MLISKSKMFKTFWILTSIVLLASATVDISKLQE |
| | | | FEEYQKFTNESLLWAPYRSNCYFGMRPRYVH |
| | | | ESPLIMGIMWFNSLSQDGLHS |
| 25 | ER | ScGLS1-l | MLISKSKMFKTFWILTSIVLLASATVDISKLQE |
| | | | FEEYQKFTNESLLWAPYRSNCYFGMRPRYVH |
| | | | ESPLIMGIMWFNSLSQDGLHSLRHFATPQDKL |
| | | | QKYGWEVYDPRIGGKEVFIDEKNNLNLTVYF |
| | | | VKSKNGENWS |
| 26 | ER | ScMNS1-s | MKNSVGISIATIVANAAIYYVPWYEHFER |
| 27 | ER | ScMNS1-m | MKNSVGISIATIVAIIAAIYYVPWYEHFERKSPG |
| | | | AGEMRDRIESMFLESWRDYSKHGWGYDVYG |
| | | | PIEHTSHNMPRGNQPLGW |
| 28 | ER | ScMNS1-l | MKNSVGISIATIVAHAAIYYVPWYEHFERKSPG |
| | | | AGEMRDRIESMFLESWRDYSKHGWGYDVYG |
| | | | PIEHTSHNMPRGNQPLGWIIVDSVDTLMLMYN |
| | | | SSTLYKSEFEAEIQRSEHWINDVLDFDIDAEVN |
| | | | VFETTIRMLGGLLS |
| 29 | ER | ScSEC12-S | ANTIHIIKLPLNYANYTSMKQKISKFFTNFILIV |
| | | | LLSYILQFSYKHNLHSM |
| 30 | ER | ScSEC12-m | ANTIHIIKLPLNYANYTSMKQKISKFFTNFILIV |
| | | | LLSYILQFSYKHNLHSMLFNYAKDNFLTKRDT |
| | | | ISSPYVVDEDLHQTTLFGNHGTKTSVPSVDSIK |
| | | | VHGV |
| 31 | ER | ScSEC12-l | ANTIHIIKLPLNYANYTSMKQKISKFFTNFILIV |
| | | | LLSYILQFSYKHNLHSMLFNYAKDNFLTKRDT |
| | | | ISSPYVVDEDLHQTTLFGNHGTKTSVPSVDSIK |
| | | | VHGVHETSSVNGTEVLCTESNIINTGGAEFEIT |
| | | | NATFREIDDA |
| 32 | ER | PpSEC12-s | PRKIFNYFILTVFMAILAIVLQWSIENGH |
| 33 | cis-Golgi | PpOCH1-s | MAKADGSLLYYNPHNPPRRYYFYMAIFAVSVI |
| | | | CVLYGPSQQLSSPKIDYD |
| 34 | cis-Golgi | PpOCH1-m | MAKADGSLLYYNPHNPPRRYYFYMAIFAVSVI |
| | | | CVLYGPSQQLSSPKIDYDPLTLRSLDLKTLEAP |
| | | | SQLSPGTVEDNLRRQ |
| 35 | cis-Golgi | PpOCH1-l | MAKADGSLLYYNPHNPPRRYYFYMAIFAVSVI |
| | | | CVLYGPSQQLSSPKIDYDPLTLRSLDLKTLEAP |
| | | | SQLSPGTVEDNLRRQLEFHFPYRSYEPFPQHI |
| | | | WQTWKVSPSDSSFPKNFKDLGESWLQRSPNY |
| | | | DHFVIP |
| 36 | cis-Golgi | ScMNN9-S | MSLSLVSYRLRKNPWVNIFLPVLAIFLIYIIFFQ |
| | | | RDQSLL |
| 37 | cis-Golgi | ScMNN9-m | MSLSLVSYRLRKNPWVNIFLPVLAIFLIYIIFFQ |
| | | | RDQSLLGLNGQSISQHKWAHEKENTFYFPFTK |
| | | | KYKMPKYSYKKKSGWLFNDHVEDII |
| 38 | cis-Golgi | ScMNN9-l | MSLSLVSYRLRKNPWVNIFLPVLAIFLIYIIFFQ |
| | | | RDQSLLGLNGQSISQHKWAHEKENTFYFPFTK |
| | | | KYKMPKYSYKKKSGWLFNDHVEDIIPEGHIA |
| | | | HYDLNKLHSTSEAAVNKEHILILTPMQTFHQQ |
| | | | YWDNLLQLNYPRELIELGFITPRTA |
| 39 | cis-Golgi | ScVAN1-s | MGMFFNLRSNIKKKAMDNGLSLPISRNGSSNN |
| | | | IKDKRSEHNSNSLKGKYRYQPRSTPSKFQLTV |
| | | | SITSLIIIAVLSLYLFISFLSGMGIGVST |
| 40 | cis-Golgi | ScVAN1-m | MGMFFNLRSNIKKKAMDNGLSLPISRNGSSNN |
| | | | IKDKRSEHNSNSLKGKYRYQPRSTPSKFQLTV |
| | | | SITSLIIIAVLSLYLFISFLSGMGIGVSTQNGRS |
| 41 | cis-Golgi | ScVAN1-l | MGMFFNLRSNIKKKAMDNGLSLPISRNGSSNN |
| | | | IKDKRSEHNSNSLKGKYRYQPRSTPSKFQLTV |
| | | | SITSLIIIAVLSLYLFISFLSGMGIGVSTQNGRSL |
| | | | LGSSKSSENYKTIDLEDEEYYDYDFEDIDPEVIS |
| | | | KFDDGVQHYLISQFGSEVLTPKDDEKYQRELN |
| | | | MLFDSTVEEYDLSNFEGAPNGLETRDHILLCIP |
| | | | LRNAA |
| 42 | cis-Golgi | ScANP1-S | MKYNNRKLSFNPTTVSIAGTLLTVFFLTRLVLS |
| | | | FFSISLFQLVTFQGIFKPYVPDFKNTP |
| 43 | cis-Golgi | ScANP1-m | MKYNNRKLSFNPTTVSIAGTLLTVFFLTRLVLS |
| | | | FFSISLFQLVTFQGIFKPYVPDFKNTPSVEFYDL |
| | | | RNYQGNKDGWQQGDR |
| 44 | cis-Golgi | ScANP1-l | MKYNNRKLSFNPTTVSIAGTLLTVFFLTRLVLS |
| | | | FFSISLFQLVTFQGIFKPYVPDFKNTPSVEFYDL |
| | | | RNYQGNKDGWQQGDRILFCVPLRDASEHLPM |
| | | | FFNHLNT |
| 45 | cis-Golgi | ScHOC1-s | MAKTTKRASSFRRLMIFAIIALISLAFGVRYLF |
| | | | H |
| 46 | cis-Golgi | ScHOC1-m | MAKTTKRASSFRRLMIFAIIALISLAFGVRYLF |
| | | | HNSNATDLQKILQNLPKEISQSINSANNIQSSDS |
| | | | DLVQHFESLAQEIRHQQ |
| 47 | cis-Golgi | ScHOC1-l | MAKTTKRASSFRRLMIFAIIALISLAFGVRYLF |
| | | | HNSNATDLQKILQNLPKEISQSINSANNIQSSDS |
| | | | DLVQHFESLAQEIRHQQEVQAKQFDKQRKILE |
| | | | KKIQDLKQTPPEATLRERIAMTFPYDSHVKFP |
| | | | AFIWQTWSNDEGPE |
| 48 | cis-Golgi | ScMNN10-s | MSSVPYNSQLPISNHLEYDEDEKKSRGSKLGL |
| | | | KYKMIYWRKTLCSSLARWRKLILLISLALFLFI |
| | | | WISDSTIS |
| 49 | cis-Golgi | ScMNN10-m | MSSVPYNSQLPISNHLEYDEDEKKSRGSKLGL |
| | | | KYKMIYWRKTLCSSLARWRKLILLISLALFLFI |
| | | | WISDSTISRNPSTTSFQGQNSNDNKLSNTGSSIN |
| | | | SKRYVPPYSKRSRWSFWNQDPR |
| 50 | cis-Golgi | ScMNN10-l | MSSVPYNSQLPISNHLEYDEDEKKSRGSKLGL |
| | | | KYKMIYWRKTLCSSLARWRKLILLISLALFLFI |
| | | | WISDSTISRNPSTTSFQGQNSNDNKLSNTGSSIN |
| | | | SKRYVPPYSKRSRWSFWNQDPRIVIILAANEG |
| | | | GGVLRWKNEQEWAIEGISIENKKAYAKRHGY |
| | | | ALTIKDLTTS |
| 51 | cis-Golgi | ScMNN11-S | MAIKPRTKGKTYSSRSVGSQWFNRLGFKQNK |
| | | | YGTCKFLSIITAFVFILYFFS |
| 52 | cis-Golgi | ScMNN11-m | MAIKPRTKGKTYSSRSVGSQWENRLGFKQNK |
| | | | YGTCKFLSIITAFVFILYFFSNRFYPISRSAGASY |
| | | | SPSHGLYINEIPASSRLIYPHVEHVPVLKQMTV |
| | | | RG |
| 53 | cis-Golgi | ScMNN11-l | MAIKPRTKGKTYSSRSVGSQWFNRLGFKQNK |
| | | | YGTCKFLSIITAFVFILYFFSNRFYPISRSAGASY |
| | | | SPSHGLYINEIPASSRLIYPHVEHVPVLKQMTV |
| | | | RGLYITRLEVDGSKRLILKPEENALTDEEKKK |
| | | | TTDQILLVKHSFLDHGKLVYRKSNDAPEVVVV |
| | | | TL |
| 54 | medial- | ScKRE2-S | MALFLSKRLLRFTVIAGAVIVLLLTLNSNSRTQ |
| | Golgi | | QYIPSSISAAFDFTSGSISPEQQVI |
| 55 | medial- | ScKRE2-m | MALFLSKRLLRFTVIAGAVIVLLLTLNSNSRTQ |
| | Golgi | | QYIPSSISAAFDFTSGSISPEQQVISEENDAKKLE |
| | | | QSALNSEASEDS |
| 56 | medial- | ScKRE2-l | MALFLSKRLLRFTVIAGAVIVLLLTLNSNSRTQ |
| | Golgi | | QYIPSSISAAFDFTSGSISPEQQVISEENDAKKLE |
| | | | QSALNSEASEDSEAMDEESKALKAAAEKADAP |
| | | | ID |
| 57 | medial- | PpKTR1-s | MAGATRINSRVVRFAIFASILVLLGFILSRGSAT |
| | Golgi | | SYSLPSGLTSDTSQSTGSSPKSESKPSSQGSSGA |
| | | | TELK |
| 58 | medial- | PpKTR1-m | MAGATRINSRVVRFAIFASILVLLGFILSRGSAT |
| | Golgi | | SYSLPSGLTSDTSQSTGSSPKSESKPSSQGSSGA |
| | | | TELKKTYTTDGKEKATFVSLARNSDVWSLASS |
| | | | IRHVEDRFNHKF |
| 59 | medial- | PpKTR1-l | MAGATRINSRVVRFAIFASILVLLGFILSRGSAT |
| | Golgi | | SYSLPSGLTSDTSQSTGSSPKSESKPSSQGSSGA |
| | | | TELKKTYTTDGKEKATFVSLARNSDVWSLASS |
| | | | IRHVEDRFNHKFHYDWVFLNDEEFSDEFKRVT |
| | | | SALTSGKAKYGLIPKEHWSFPEWIDKERAAKT |
| | | | RKEMAAKKVIYGDSISYRHMCRFESGFFFRHE |
| 60 | medial- | PpKTR3-S | MMRARLSLERVNLSFITSVFLASVAVLFIS |
| | Golgi | | |
| 61 | medial- | PpKTR3-m | MMRARLSLERVNLSFITSVFLASVAVLFISLEM |
| | Golgi | | PKVLARDRQILKLKLGFMGSGLQKGSLETSG |
| | | | NIENTESNINS |
| 62 | medial- | PpKTR3-l | MMRARLSLERVNLSFITSVFLASVAVLFISLEM |
| | Golgi | | PKVLARDRQILKLKLGFMGSGLQKGSLETSG |
| | | | NIENTESNINSQTTQHIGTIGASNERANATFYTL |
| | | | CRNEELYQMLETVQNYEDRENSKFKYDWVFL |
| | | | NDYPFTDEFKRVISHAISGEAKFGQVPASHWR |
| | | | FP |
| 63 | medial- | PpKRE2-s | MVHIGFRSLKAVFILALSSLILYGIVTTFDG |
| | Golgi | | |
| 64 | medial- | PpKRE2-m | MVHIGFRSLKAVFILALSSLILYGIVTTFDGSRA |
| | Golgi | | SRYQPPYVNHSQDPLYHSGNSYNRENATFVTL |
| | | | CRNEDLYSIIQSIKKVED |
| 65 | medial- | PpKRE2-l | MVHIGFRSLKAVFILALSSLILYGIVTTFDGSRA |
| | Golgi | | SRYQPPYVNHSQDPLYHSGNSYNRENATFVTL |
| | | | CRNEDLYSIIQSIKKVEDRENNKFAYDWVFLN |
| | | | EVPFTDEFKERTSVLISGQAKYGLIPKEHWSYP |
| | | | DYIDQERAAESRRQLEDQH |
| 66 | medial- | ScKTR1-S | MAKIMIPASKQPVYKKLGLLLVAVFTVYVFFH |
| | Golgi | | GAQYARG |
| 67 | medial- | ScKTR1-l | MAKIMIPASKQPVYKKLGLLLVAVFTVYVFFH |
| | Golgi | | GAQYARGSAPSPKYSTVLSSGSGYKYSKVELP |
| | | | KYTGPREKATFVTLVRNRDLYSLAESIKSVED |
| | | | RFNSKFNYDWVFLNDEEFTDEFKNVTSALVSG |
| | | | TTKYGVIPKEHWSFPEWIDEEKAAQVR |
| 68 | medial- | ScKTR2-S | MQICKVFLTQVKKLLFVSLLFCLIAQTCWLAL |
| | Golgi | | VPYQRQLS |
| 69 | medial- | ScKTR2-m | MQICKVFLTQVKKLLFVSLLFCLIAQTCWLAL |
| | Golgi | | VPYQRQLSLDSYFFRRSREVSSRYDFTRRRHM |
| | | | NQTLKLSSNTYNDEPLNKTKGIKNQRENATLL |
| | | | MLVR |
| 70 | medial- | ScKTR2-l | MQICKVFLTQVKKLLFVSLLFCLIAQTCWLAL |
| | Golgi | | VPYQRQLSLDSYFFRRSREVSSRYDFTRRRHM |
| | | | NQTLKLSSNTYNDEPLNKTKGIKNQRENATLL |
| | | | MLVRNWELSGALRSMRSLEDRENKNYQYDW |
| | | | TFLNDVPFDQEFIEATTAMASGRTQYALIPAED |
| | | | WNRPSWINETL |
| 71 | medial- | ScMNN2-S | MLLTKRFSKLFKLTFIVLILCGLFVITNKYMDE |
| | Golgi | | NTS |
| 72 | medial- | ScMNN2-m | MLLTKRFSKLFKLTFIVLILCGLFVITNKYMDE |
| | Golgi | | NTSVKEYKEYLDRYVQSYSNKYSSSSDAASAD |
| | | | DSTPLRDNDEAGNEKLKSFYNNVENFLMVDSP |
| 73 | medial- | ScMINN2-l | MLLTKRFSKLFKLTFIVLILCGLFVITNKYMDE |
| | Golgi | | NTSVKEYKEYLDRYVQSYSNKYSSSSDAASAD |
| | | | DSTPLRDNDEAGNEKLKSFYNNVFNFLMVDSP |
| | | | KGSTAKQYNEACLLKGDIGDRPDHYKDLYKL |
| | | | SAKELSKCLELSPDEVASLTKS |
| 74 | medial- | ScMNN5-S | MLIRLKKRKILQVIVSAVVLILFFCSVHNDVSS |
| | Golgi | | SW |
| 75 | medial- | ScMNN5-m | MLIRLKKRKILQVIVSAVVLILFFCSVHNDVSS |
| | Golgi | | SWLYGKKLRLPVLTRSNLKNNFYTTLVQAIVE |
| | | | NKPADSSPDLSKLIGAEGCS |
| 76 | medial- | ScMNN5-l | MLIRLKKRKILQVIVSAVVLILFFCSVHNDVSS |
| | Golgi | | SWLYGKKLRLPVLTRSNLKNNFYTTLVQAIVE |
| | | | NKPADSSPDLSKLIGAEGCSFANNVAAHDSGH |
| | | | DSDLSYESLSKCYNLNKTVQESLREVHSKFTD |
| | | | TLSGKLNFSIPQREALFSGSE |
| 77 | medial- | ScYUR1-S | MAKGGSLYIVGIFLPIWTFMIYIFGKELFLIRK |
| | Golgi | | YQK |
| 78 | medial- | ScYUR1-m | MAKGGSLYIVGIFLPIWTFMIYIFGKELFLIRK |
| | Golgi | | YQKIDSTYTALSQRVKEQYDTSRRRNYFPKVK |
| | | | LSRNSYDDYTLNYTRQNDSDSFHLR |
| 79 | medial- | ScYUR1-I | MAKGGSLYIVGIFLPIWTFMIYIFGKELFLIRK |
| | Golgi | | YQKIDSTYTALSQRVKEQYDTSRRRNYFPKVK |
| | | | LSRNSYDDYTLNYTRQNDSDSFHLRENATILM |
| | | | LVRNSELEGALDSMRSLEDRENNKYHYDWTF |
| | | | LNDVPFDQDFIEATTSMASGKT |
| 80 | late Golgi | ScMNN1-S | MLALRRFILNQRSLRSCTIPILVGALIIILVLFQ |
| | | | LVTHRNDA |
| 81 | late Golgi | ScMNN1-m | MLALRRFILNQRSLRSCTIPILVGALIILVLFQ |
| | | | LVTHRNDALIRSSNVNSTNKKTLKDADPKVLI |
| | | | EAFGSPEVDPVDTIPVSPLELVPFYDQ |
| 82 | late Golgi | ScMNN1-l | MLALRRFILNQRSLRSCTIPILVGALIILVLFQ |
| | | | LVTHRNDALIRSSNVNSTNKKTLKDADPKVLI |
| | | | EAFGSPEVDPVDTIPVSPLELVPFYDQSIDTKRS |
| | | | SSWLINKKGYYKHFNELSLTDRCKFYFRTLYT |
| | | | LDDEWTNSVKKLEYSINDNEG |
| 83 | late Golgi | ScMNN6-S | MHVLLSKKIARFLLISFVFVLALMVTINHP |
| 84 | late Golgi | ScMNN6-m | MHVLLSKKIARFLLISFVFVLALMVTINHPKT |
| | | | KQMSEQYVTPYLPKSLQPIAKISAEEQRRIQSE |
| | | | QEEAELKQSL |
| 85 | late Golgi | ScMNN6-l | MHVLLSKKIARFLLISFVFVLALMVTINHPKT |
| | | | KQMSEQYVTPYLPKSLQPIAKISAEEQRRIQSE |
| | | | QEEAELKQSLEGEAIRNATVNAIKEKIKSYGG |
| | | | NETTLGFMVPSYINHRGSPPKACFVSLITERDS |
| | | | MTQILQSIDEVQVKENKNFAYPWVFISQGE |

TABLE 5 provides exemplary promoter sequences.

TABLE 5
Exemplary Promoter Sequences

| SEQ ID | Promoter | Promoter | |
|---|---|---|---|
| NO: | name | type | Sequence |
| 86 | aoxl | Inducible | gatctaacatccaaagacgaaaggttgaatgaaacctttt |
| | | | tgccatccgacatccacaggtccattctcacacataagtg |
| | | | ccaaacgcaacaggaggggatacactagcagcagaccgtt |
| | | | gcaaacgcaggacctccactcctcttctcctcaacaccca |
| | | | cttttgccatcgaaaaaccagcccagttattgggcttgat |
| | | | tggagctcgctcattccaattccttctattaggctactaa |
| | | | caccatgactttattagcctgtctatcctggcccccctgg |
| | | | cgaggttcatgtttgtttatttccgaatgcaacaagctcc |
| | | | gattacacccgaacatcactccagatgagggctttctgag |
| | | | tgtggggtcaaatagtttcatgttccccaaatggcccaaa |
| | | | actgacagtttaaacgctgtcttggaacctaatatgacaa |
| | | | aagcgtgatctcatccaagatgaactaagtttggttcgtt |
| | | | gaaatgctaacggccagttggtcaaaaagaaacttccaaa |
| | | | agtcggcataccgtttgtcttgtttggtattgattgacga |
| | | | atgctcaaaaataatctcattaatgcttagcgcagtctct |
| | | | ctatcgcttctgaaccccggtgcacctgtgccgaaacgca |
| | | | aatggggaaacacccgctttttggatgattatgcattgtc |
| | | | tccacattgtatgcttccaagattctggtgggaatactgc |
| | | | tgatagcctaacgttcatgatcasaatttaactgttctaa |
| | | | cccctacttgacagcaatatataaacagaaggaagctgcc |
| | | | ctgtcttaaacctttttttttatcatcattattagcttac |
| | | | tttcataattgcgactggttccaattgacaagcttttgat |
| | | | tttaacgacttttaacgacaacttgagaagatcaaaaaac |
| | | | aactaattattcgaaacg |
| 87 | amyB | Inducible | ggcttggggttgagcatgtttatgaatgtttatgcttatg |
| | | | ttctctcatcctgtatatatgcagaagcggaaaggagtag |
| | | | cttgaggactgatgggtagcggtgattcttcagtattaaa |
| | | | attatggatcttgcaatgcattcttttgttcgtataaagt |
| | | | ctttcctaagatcataataccagcaatgaccgttgccatt |
| | | | cactttctgcccgccactgagcctgaccgaagctatccat |
| | | | ccgaggggccccaagccctgtagccttaacattcttccga |
| | | | agtcactgcagtgttattacctgtctggaacctatgtcat |
| | | | attaggatagaatagagaagatgctgactgatatagctca |
| | | | gcactctgaagcctcaggcagtccacaggcccagggatat |
| | | | gaggatttgacaaggaaaggtgtgacgtactcctaccccg |
| | | | tcctcgcgactatttctgcagtctgagaaaagaagacgca |
| | | | tagccttgaacatagggcattggctgcagtgaaacgcccc |
| | | | agttactacttatcgcagcatacgtgacgagaactcagtg |
| | | | gttatccttccagtatagtgtagaacatctgaatacatca |
| | | | gaacagaagcccgatacctcccgtaactatccattccatg |
| | | | acctaaagatggtacaaggaaaaatttgcttgctgtttca |
| | | | tgatctagctcctgagacacggagattcctcccttcctat |
| | | | ggtcaaaaaggcagcccattcccaaaggagggcttcatcg |
| | | | cttgatgagttctcgaatacaggctaatatatattatcat |
| | | | catcatatcgacacctaccttgaccaactgtaatatagtg |
| | | | tttccttcagtatgtgacaacgaagcgtactcttggggca |
| | | | ccacagcttgtgctcgcggcgaattccagtcgcaatccga |
| | | | aacttgcgggaagatagaatgacaacctacagtcacagat |
| | | | ctattaagtgtgatttaaatgggctatattggccttg |
| 88 | lac4 | Inducible | ctagtaaccaaaggaaaggaacagatagataaaattccga |
| | | | gactgtcaaattaggtttttttctttttttttggcgggag |
| | | | tcagtgggccgaaatatgttcttggcctagaacttaatct |
| | | | ggtttgatcatgccaatacttgcctgagtgcccgactttt |
| | | | tgcccacccttttgccttctgtctatccttcaaaacccac |
| | | | ctgttttccagccgtatcttcgctcgcatctacacatact |
| | | | gtgccatatcttgtgtgtagccggacgtgactatgaccaa |
| | | | aaacaaacaaggagaactgttcgccgatttgtaacactcc |
| | | | tgcatccatccaagtgggtatgcgctatgcaatgttaagc |
| | | | taggtcaggtcagaccaggtccaaggacagcaacttgact |
| | | | gtatgcaacctttaccatctttgcacagaacatacttgta |
| | | | gctagctagttacacttatggaccgaaaaggcaccccacc |
| | | | atgtctgtccggctttagagtacggccgcagaccgctgat |
| | | | ttgccttgccaagcagtagtcacaatgcatcgcatgagca |
| | | | cacgggcacgggcacgggcacaggaaccattggcaaaaat |
| | | | accagatacactataccgacgtatatcaagcccaagttta |
| | | | aaattcctaaatttccgcggggatcgactcataaaatagt |
| | | | aaccttctaatgcgtatctattgactaccaaccattagtg |
| | | | tggttgcagaaggcggaattctcccttcttcgaattcagc |
| | | | ttgctttttcattttttattttccatttttcagtttttgt |
| | | | ttgtgtcgaatttagccagttgcttctccaagatgaaaaa |
| | | | aacccctgcgcagtttctgtgctgcaagatcctaatcgac |
| | | | ttttccaccccccacaaaagtaaatgttcttttgttacat |
| | | | tcgcgtgggtagctagctccccgaatcttcaaaggactta |
| | | | gggactgcactacatcagagtgtgttcacctggtttgctg |
| | | | cctggtttgaaagaaaagagcagggaactcgcgggttccc |
| | | | ggcgaataatcatgcgatagtcctttggccttccaagtcg |
| | | | catgtagagtagacaacagacagggagggcaggaaggatc |
| | | | tttcactgagatcctgtatcttgttgggtaagtcggatga |
| | | | aaggggaatcgtatgagattggagaggatgcggaagaggt |
| | | | aacgccttttgttaacttgtttaattattatggggcaggc |
| | | | gagagggggaggaatgtatgtgtgtgaggcgggcgagacg |
| | | | gagccatccaggccaggtagaaatagagaaagccgaatgt |
| | | | tagacaatatggcagcgtagtagagtaggtaggtaggcaa |
| | | | gtactgctagcaaagaggagaagggtaagctcactcttcg |
| | | | cattccacaccgttagtgtgtcagtttgaacaaaaaaaca |
| | | | atcatcataccaattgatggactgtggactggcttttgga |
| | | | acggcttttcggactgcgattattcgtgaggaatcaaggt |
| | | | aggaatttggtcatatttacggacaacagtgggtgattcc |
| | | | catatggagtaggaaaacgagatcatggtatcctcagata |
| | | | tgttgcggaattctgttcaccgcaaagttcagggtgctct |
| | | | ggtgggtttcggttggtctttgctttgcttctcccttgtc |
| | | | ttgcatgttaataatagcctagcctgtgagccgaaactta |
| | | | gggtaggcttagtgttggaacgtacatatgtatcacgttg |
| | | | acttggtttaaccaggcgacctggtagccagccataccca |
| | | | cacacgttttttgtatcttcagtatagttgtgaaaagtgt |
| | | | agcggaaatttgtggtccgagcaacagcgtctttttctag |
| | | | tagtgcggtcggttacttggttgacattggtatttggact |
| | | | ttgttgctacaccattcactacttgaagtcgagtgtgaag |
| | | | ggtatgatttctagtggtgaacacctttagttacgtaatg |
| | | | ttttcattgctgttttacttgagatttcgattgagaaaaa |
| | | | ggtatttaatagctcgaatcaatgtgttatcattgtgaag |
| | | | atgttcttccctaactcgaaaggtatatgaggcttgtgtt |
| | | | tcttaggagaattattattcttttgttatgttgcgcttgt |
| | | | agttggaaaaggtgaagagacaaaagc |
| 89 | T7 | Inducible | taatacgactcactatagg |
| 90 | GAP in | Constitutive | tttttgtagaaatgtcttggtgtcctcgtccaatcaggta |
| | | P. pastoris | gccatctctgaaatatctggctccgttgcaactccgaacg |
| | | | acctgctggcaacgtaaaattctccggggtaaaacttaaa |
| | | | tgtggagtaatggaaccagaaacgtctcttcccttctctc |
| | | | tccttccaccgcccgttaccgtccctaggaaattttactc |
| | | | tgctggagagcttcttctacggcccccttgcagcaatgct |
| | | | cttcccagcattacgttgcgggtaaaacggaggtcgtgta |
| | | | cccgacctagcagcccagggatggaaaagtcccggccgtc |
| | | | gctggcaataatagcgggcggacgcatgtcatgagattat |
| | | | tggaaaccaccagaatcgaatataaaaggcgaacaccttt |
| | | | cccaattttggtttctcctgacccaaagactttaaattta |
| | | | atttatttgtccctatttcaatcaattgaacaactat |
| 91 | tefl | Constitutive | ATAGCTTCAAAATGTCTCTACTCCTTTTTTACTCTTCCAG |
| | | | ATTTTCTCGGACTCCGCGCACCGCCGTACCACTTCAAAAC |
| | | | ACCCAAGCAACAGCATACTAAATTCCCCCTCCTCCTTCCT |
| | | | CTAGGGTGCCGTTAATTACCCGTACTAAAGGTTTGGAAAA |
| | | | GGAAAAAGAGACCGCCTCGTCCCTTTTTCTTCGTCGGAGA |
| | | | AGGCAATAAAAATTTTTATCACGTTTCTTTCTCTTGAAAA |
| | | | CTTTTTTTTCGATTTTTGTCTCTTTCGACGACCTCCCATT |
| | | | GATATTTGAGTTAACAAACGGTCTTCAATTTCTCAAGTTI |
| | | | CAGCTTCATTTTTCCTGTTCTATTACAACTTTTTTTACTT |
| | | | CTTGCTCATTGGAAAGAAAGCATAGCAATCTAATCTAAGT |
| | | | TTTAATTACAAA |
| 92 | adh1 | Constitutive | tgtacaatatggacttcctcttttctggcaaccaaaccca |
| | | | tacatcgggattcctataataccttcgttggtctccctaa |
| | | | catgtaggtggcggaggggagatatacaatagaacagata |
| | | | ccagacaagacataatgggctaaacaagactacaccaatt |
| | | | acactgcctcattgatggtggtacataacgaactaatact |
| | | | gtagccctagacttgatagccatcatcatatcgaagtttc |
| | | | actaccctttttccatttgccatctattgaagtaataata |
| | | | ggcgcatgcaacttcttttctttttttttcttttctctct |
| | | | cccccgttgttgtctcaccatatccgcaatgacaaaaaaa |
| | | | tgatggaagacactaaaggaaaaaattaacgacaaagaca |
| | | | gcaccaacagatgtcgttgttccagagctgatgaggggta |
| | | | tctcgaagcacacgaaactttttccttccttcattcacgc |
| | | | acactactctctaatgagcaacggtatacggcccttcctt |
| | | | ccagttacttgaatttgaaataaaaaaaagtttgctgtct |
| | | | tgctatcaagtataaatagacctgcaattattaatctttt |
| | | | gtttcctcgtcattgttctcgttccctttcttccttgttt |
| | | | ctttttctgcacaatatttcaagctataccaagcatacaa |
| | | | tcaagcaattccagatctgccacc |
| 415 | GAP1 in | Constitutive | tttatctttttttagtatagagtttgtgtgtttaaagctt |
| | | K. lactis | gtttatgtttcaattgaaactaftgtttttgcttgatgat |
| | | | gatgtagggcaaaacagaaacaacattgtacaaatggata |
| | | | tagagagacctactacaaaagtggatggattgtcagaatc |
| | | | gaccgggtgtttatatacatattccagaagaccaaatggc |
| | | | tggtctaattgtatacaagtaagacagctgatttctagct |
| | | | ggtgtgaaaatagcacactttgagaaaaatacataccacc |
| | | | ctgcaacaggtgttaaccgtttaagacctagaaataaccg |
| | | | cagtatataaagatcaatcgaatacttgggttggtgaaaa |
| | | | gcaacccaaaatacatcatctgctcatatgattgtgatct |
| | | | gattgtgaatagatcaatacactactagaatagaagaaca |
| | | | tcacacgctatttttttttcctcttctgtcctactcggag |
| | | | aaacagaaagtatcaggtcaccaccggacatgtcatacta |
| | | | cctcggaagattcccaattacgcacgtaaaaactaaaacg |
| | | | agcccccaccaaagaacaaaaaagaaggtgctgggccccc |
| | | | actttcttcccttgcacgtgataggaagatggctacagaa |
| | | | acaagaagatggaaatcgaaggaaagagggagactggaag |
| | | | ctgtaaaaactgaaatgaaaaaaaaaaaaaaaaaaaaaaa |
| | | | caagaagctgaaaatggaagactgaaatttgaaaaatggt |
| | | | aaaaaaaaaaaagaaacacgaagctaaaaacctggattcc |
| | | | attttgagaagaagcaagaaaggtaagtatggtaacgacc |
| | | | gtacaggcaagcgcgaaggcaaatggaaaagctggggagt |
| | | | ccggaagataatcatttcatcttcttttgttagaacagaa |
| | | | cagtggatgtccctcatctcggtaacgtattgtccatgcc |
| | | | ctagaactctctgtccctaaaaagaggacaaaaacccaat |
| | | | ggtttccccagcttccagtggagc |
| | | | c |

TABLE 6 provides exemplary Type II Transmembrane Proteins.

TABLE 6
Exemplary Type II Transmembrane Proteins

| SEQ ID | | |
|---|---|---|
| NO: | Protein | Sequence |
| 155 | ScKRE2 | MALFLSKRLLRFTVIAGAVIVLLLTLNSNSRTQQYIPSSISA |
| | | AFDFTSGSISPEQQVISEENDAKKLEQSALNSEASEDSEAM |
| | | DEESKALKAAAEKADAPIDTKTTMDYITPSFANKAGKPK |
| | | ACYVTLVRNKELKGLLSSIKYVENKINKKFPYPWVFLND |
| | | EPFTEEFKEAVTKAVSSEVKFGILPKEHWSYPEWINQTKA |
| | | AEIRADAATKYIYGGSESYRHMCRYQSGFFWRHELLEEY |
| | | DWYWRVEPDIKLYCDINYDVFKWMQENEKVYGFTVSIH |
| | | EYEVTIPTLWQTSMDFIKKNPEYLDENNLMSFLSNDNGK |
| | | TYNLCHFWSNFEIANLNLWRSPAYREYFDTLDHQGGFFY |
| | | ERWGDAPVHSIAAALFLPKDKIHYFSDIGYHHPPYDNCPL |
| | | DKEVYNSNNCECDQGNDFTFQGYSCGKEYYDAQGLVKP |
| | | KNWKKFRE |
| 156 | PpKRE2 | MVHIGFRSLKAVFILALSSLILYGIVTTFDGSRASRYQPPY |
| | | VNHSQDPLYHSGNSYNRENATFVTLCRNEDLYSIIQSIKK |
| | | VEDRFNNKFAYDWVFLNEVPFTDEFKERTSVLISGQAKY |
| | | GLIPKEHWSYPDYIDQERAAESRRQLEDQHVVYGGLESY |
| | | RHMCRENSGFFYKHPLMLDYRYYWRVEPEIEILCDVETD |
| | | LFRYMRENNKTYGFTISIHEFEKTIPTLWETTKEFMKQNP |
| | | SYIAENNLMNFISDDNGKTYNLCHFWSNFEVADMDFWRS |
| | | DVYEKYFKFLDDTGKFFYERWGDAPVHSLAVSLFLPKEK |
| | | VHFFNEVGYKHSVYSMCPIDKDIWKNRKCYCDPNTDFTF |
| | | RGYSCGRQYYKATGLTRPSNWKDYD |
| 157 | ScMNN2 | MLLTKRFSKLFKLTFIVLILCGLFVITNKYMDENTSVKEY |
| | | KEYLDRYVQSYSNKYSSSSDAASADDSTPLRDNDEAGNEK |
| | | LKSFYNNVFNFLMVDSPKGSTAKQYNEACLLKGDIGDRP |
| | | DHYKDLYKLSAKELSKCLELSPDEVASLTKSHKDYVEHIA |
| | | TLVSPKGTYKGSGIATVGGGKFSLMAFLIIKTLRNMGTTL |
| | | PVEVLIPPGDEGETEFCNKILPKYNSKCIYVSDILPRETIEK |
| | | FVFKGYQFKSLALIASSFENLLLLDADNFPIKPLDNIFNEE |
| | | PYVSTGLVMWPDFWRRTTHPLYYDIAGIAVDKKKRVRN |
| | | SRDDITPPAVYTKDLKDLSDVPLSDLDGTIPDVSTESGQL |
| | | MINKTKHLATALLSLFYNVNGPTWYYPIFSQKAAGEGDK |
| | | ETFIAAANFYGLSFYQVRTRTGVEGYHDEDGFHGVAMLQ |
| | | HDFVQDYGRYLNAMESIGNKYGGTKSADAIKFDKNYSLE |
| | | KYTEEFFDNEDLNAKNHVDVMFIHSNFPKFDPYDLSKSNF |
| | | LTTNGKPARSYTALKKVKNYDIELENFKVLNEYVCVNKN |
| | | PFKYLDDLLGQDKTEWKRVCGYITDRLAFLESTHDKAIA |
| | | GK |
| 158 | ScMNN1 | MLALRRFILNQRSLRSCTIPILVGALIILVLFQLVTHRNDA |
| | | LIRSSNVNSTNKKTLKDADPKVLIEAFGSPEVDPVDTIPVS |
| | | PLELVPFYDQSIDTKRSSSWLINKKGYYKHFNELSLTDRC |
| | | KFYFRTLYTLDDEWTNSVKKLEYSINDNEGVDEGKDANG |
| | | NPMDEKSERLYRRKYDMFQAFERIRAYDRCFMQANPVNI |
| | | QEIFPKSDKMSKERVQSKLIKTLNATFPNYDPDNFKKYDQ |
| | | FEFEHKMFPFINNFTTETFHEMVPKITSPFGKVLEQGFLP |
| | | KFDHKTGKVQEYFKYEYDPSKTFWANWRDMSAKVAGR |
| | | GIVLSLGSNQFPLAVKFIASLRFEGNTLPIQVVYRGDELSQ |
| | | ELVDKLIYAARSPDFKPVENNYDNSTNVPQEIWFLDVSNT |
| | | IHPKWRGDFGSYKSKWLVVLLNLLQEFVFLDIDAISYEKI |
| | | DNYFKTTEYQKTGTVFYRERALRENVNERCIARYETLLP |
| | | RNLESKNFQNSLLIDPDHALNECDNTLTTEEYIFKAFFHH |
| | | RRQHQLEAGLFAVDKSKHTIPLVLAAMIHLAKNTAHCTH |
| | | GDKENFWLGFLAAGHTYALQGVYSGAIGDYVKKTDLNG |
| | | KRQEAAVEICSGQIAHMSTDKKTLLWVNGGGTFCKHDN |
| | | AAKDDWKKDGDFKKFKDQFKTFEEMEKYYYITPISSKYV |
| | | ILPDPKSDDWHRASAGACGGYIWCATHKTLLKPYSYNHR |
| | | TTHGELITLDEEQRLHIDAVNTVWSHANKDNTRSFTEEEI |
| | | KELENSRHEQS |
| 159 | ScMNN6 | MHVLLSKKIARFLLISFVFVLALMVTINHPKTKQMSEQY |
| | | VTPYLPKSLQPIAKISAEEQRRIQSEQEEAELKQSLEGEAI |
| | | RNATVNAIKEKIKSYGGNETTLGFMVPSYINHRGSPPKAC |
| | | FVSLITERDSMTQILQSIDEVQVKENKNFAYPWVFISQGEL |
| | | DGMKQEMIRQAITDSMNGDPELINIKFAEIPADEWVYPE |
| | | WIDENKAAESLISLANVPDGDSRAVRYQARYFAGFFWRH |
| | | PVLDEFDWYWRVDPGIKLYCDIDHDLFRWMQDEGKVFG |
| | | FTLSMSEAKEANEKIWDVTKKFAKDFPKFISENNFKSFIT |
| | | KKDSEDENNCEFTSNFEIGNLNFYRSPAYRKFFNYIDEEG |
| | | GIFYWKWSDSIIHTIGLSMLLPKDKIHFFENIGFHYDKYN |
| | | NCPLNDDIWNQYNCNCDQGNDFTFRSGSCGGHYFDIMK |
| | | KDKPEGWDRLP |

TABLE 7 provides exemplary Type II Transmembrane Domains.

TABLE 7
Exemplary Type II Transmembrane Domains

| SEQ | Type II | |
|---|---|---|
| ID | Transmembrane | |
| NO: | Domain | Sequence |
| 181 | ScKRE2 | FTVIAGAVIVLLLTLNSNS |
| | transmembrane | |
| | domain | |
| 182 | ScMNN2 | LTFIVLILCGLFVITN |
| | transmembrane | |
| | domain | |
| 183 | ScMNN1 | CTIPILVGALIUILVLF |
| | transmembrane | |
| | domain | |
| 184 | ScMNN6 | IARFLLISFVFVLALMVTINH |
| | transmembrane | |
| | domain | |

TABLE 8 provides exemplary Type II Transmembrane Protein Truncates.

TABLE 8
Exemplary Type II Transmembrane Protein Truncates

| SEQ ID | | | |
|---|---|---|---|
| NO: | Name | Name | Sequence |
| 160 | ScKRE2 | ScKRE2 | MALFLSKRLLRFTVIAGAVIVLLLTLNSNSRTQ |
| | | (M1-158) | QYIPSSISAAFDFTSGSISPEQQVI |
| 161 | ScKRE2 | ScKRE2 | MALFLSKRLLRFTVIAGAVIVLLLTLNSNSRTQ |
| | | (M1-S80) | QYIPSSISAAFDFTSGSISPEQQVISEENDAKKLE |
| | | | QSALNSEASEDS |
| 162 | ScKRE2 | ScKRE2 | MALFLSKRLLRFTVIAGAVIVLLLTLNSNSRTQ |
| | | (M1-D102) | QYIPSSISAAFDFTSGSISPEQQVISEENDAKKLE |
| | | | QSALNSEASEDSEAMDEESKALKAAAEKADAPI |
| | | | D |
| 163 | PpKRE2 | PpKRE2 | MVHIGFRSLKAVFILALSSLILYGIVTTEDG |
| | | (M1-G31) | |
| 164 | PpKRE2 | PpKRE2 | MVHIGFRSLKAVFILALSSLILYGIVTTEDGSRAS |
| | | (M1-D84) | RYQPPYVNHSQDPLYHSGNSYNRENATFVTLCR |
| | | | NEDLYSIIQSIKKVED |
| 165 | PpKRE2 | PpKRE2 | MVHIGFRSLKAVFILALSSLILYGIVTTFDGSRAS |
| | | (MI-H150) | RYQPPYVNHSQDPLYHSGNSYNRENATFVTLCR |
| | | | NEDLYSIIQSIKKVEDRENNKFAYDWVFLNEVPF |
| | | | TDEFKERTSVLISGQAKYGLIPKEHWSYPDYID |
| | | | QERAAESRRQLEDQH |
| 166 | ScMNN2 | ScMNN2 | MLLTKRFSKLFKLTFIVLILCGLFVITNKYMDE |
| | | (M1-S36) | NTS |
| 167 | ScMNN2 | ScMNN2 | MLLTKRFSKLFKLTFIVLILCGLFVITNKYMDE |
| | | (M1-P97) | NTSVKEYKEYLDRYVQSYSNKYSSSSDAASADD |
| | | | STPLRDNDEAGNEKLKSFYNNVFNFLMVDSP |
| 168 | ScMNN2 | ScMNN2 | MLLTKRFSKLFKLTFIVLILCGLFVITNKYMDE |
| | | (M1-S150) | NTSVKEYKEYLDRYVQSYSNKYSSSSDAASADD |
| | | | STPLRDNDEAGNEKLKSFYNNVFNFLMVDSPK |
| | | | GSTAKQYNEACLLKGDIGDRPDHYKDLYKLSA |
| | | | KELSKCLELSPDEVASLTKS |
| 169 | ScMNN1 | ScMNN1 | MLALRRFILNQRSLRSCTIPILVGALIILVLFQL |
| | | (MI-A42) | VTHRNDA |
| 170 | ScMNN1 | ScMNN1 | MLALRRFILNORSLRSCTIPILVGALIIILVLFQL |
| | | (M1-Q93) | VTHRNDALIRSSNVNSTNKKTLKDADPKVLIEA |
| | | | FGSPEVDPVDTIPVSPLELVPFYDQ |
| 171 | ScMNN1 | ScMNN1 | MLALRRFILNORSLRSCTIPILVGALIIILVLFQL |
| | | (MI-G153) | VTHRNDALIRSSNVNSTNKKTLKDADPKVLIEA |
| | | | FGSPEVDPVDTIPVSPLELVPFYDQSIDTKRSSS |
| | | | WLINKKGYYKHFNELSLTDRCKFYFRTLYTLD |
| | | | DEWTNSVKKLEYSINDNEG |
| 172 | ScMNN6 | ScMNN6 | MHVLLSKKIARFLLISFVFVLALMVTINHP |
| | | (M1-P30) | |
| 173 | ScMNN6 | ScMNN6 | MHVLLSKKIARFLLISFVFVLALMVTINHPKTK |
| | | (M1-V85) | QMSEQYVTPYLPKSLQPIAKISAEEQRRIQSEQE |
| | | | EAELKQSLEGEAIRNATV |
| 174 | ScMNN6 | ScMNN6 | MHVLLSKKIARFLLISFVFVLALMVTINHPKTK |
| | | (MI-E160) | QMSEQYVTPYLPKSLQPIAKISAEEQRRIQSEQE |
| | | | EAELKQSLEGEAIRNATVNAIKEKIKSYGGNET |
| | | | TLGFMVPSYINHRGSPPKACFVSLITERDSMTQI |
| | | | LQSIDEVQVKENKNFAYPWVFISQGE |

### Expressed Heterologous Proteins

Recombinant host cells described herein may be engineered to express a heterologous protein comprising one or more motifs susceptible to post-translational modifications (e.g., phosphorylation). In some embodiments, such a protein is a secretory protein. The heterologous protein may be a therapeutic or nutritional, wherein the nutritional protein may be a dairy protein. In some embodiments, the dairy protein is casein or a portion thereof, wherein the casein is αs1-casein or a portion thereof, αs2-casein or a portion thereof, β-casein or a portion thereof, or κ-casein or a portion thereof. In some embodiments, the dairy protein is glycomacropeptide or a portion thereof, osteopontin or a portion thereof, or lactoferrin or a portion thereof. In some embodiments, the nutritional protein is an egg white protein, wherein the egg white protein is ovalbumin or a portion thereof.

In some embodiments, the heterologous protein can comprise, consist, or consist essentially of an amino acid sequence having at least 75%, 80%, 85%, 90%, 95%, 96%, 97%, 98%, 99%, or 100% sequence identity to the sequence set forth in SEQ ID NO. 4. In some embodiments, the heterologous protein can comprise, consist, or consist essentially of an amino acid sequence having at least 75%, 80%, 85%, 90%, 95%, 96%, 97%, 98%, 99%, or 100% sequence identity to the sequence set forth in SEQ ID NO. 5. In some embodiments, the heterologous protein can comprise, consist, or consist essentially of an amino acid sequence having at least 75%, 80%, 85%, 90%, 95%, 96%, 97%, 98%, 99%, or 100% sequence identity to the sequence set forth in SEQ ID NO. 175. In some embodiments, the heterologous protein can comprise, consist, or consist essentially of an amino acid sequence having at least 75%, 80%, 85%, 90%, 95%, 96%, 97%, 98%, 99%, or 100% sequence identity to the sequence set forth in SEQ ID NO. 176. In some embodiments, the heterologous protein can comprise, consist, or consist essentially of an amino acid sequence having at least 75%, 80%, 85%, 90%, 95%, 96%, 97%, 98%, 99%, or 100% sequence identity to the SEQ ID sequence set forth in NO. 177. In some embodiments, the heterologous protein can comprise, consist, or consist essentially of an amino acid sequence having at least 75%, 80%, 85%, 90%, 95%, 96%, 97%, 98%, 99%, or 100% sequence identity to the sequence set forth in SEQ ID NO. 178. In some embodiments, the heterologous protein can comprise, consist, or consist essentially of an amino acid sequence having at least 75%, 80%, 85%, 90%, 95%, 96%, 97%, 98%, 99%, or 100% sequence identity to the sequence set forth in SEQ ID NO. 179. In some embodiments, the heterologous protein can comprise, consist, or consist essentially of an amino acid sequence having at least 75%, 80%, 85%, 90%, 95%, 96%, 97%, 98%, 99%, or 100% sequence identity to the sequence set forth in SEQ ID NO. 180. In some embodiments, the heterologous protein can comprise, consist, or consist essentially of an amino acid sequence having at least 75%, 80%, 85%, 90%, 95%, 96%, 97%, 98%, 99%, or 100% sequence identity to the sequence set forth in SEQ ID NO. 416, 422, 417, 423, 418, 424, 419, 425, 420, 421, or 435.

The expressed heterologous protein may be a component of a protein complex, wherein the protein complex comprises a first protein and a second protein. In some embodiments, the first protein, the second protein, or both the first and second proteins of the protein complex may be the expressed heterologous protein, which may comprise one or more phosphorylation sites susceptible to phosphorylation by a serine/threonine kinase. The protein complex may further comprise four proteins, wherein each of the proteins of the protein complex comprises one or more phosphorylation sites susceptible to phosphorylation by a serine/threonine kinase.

TABLE 9 provides exemplary heterologous proteins.

TABLE 9
Exemplary Heterologous Proteins

| SEQ ID | Heterologous | |
|---|---|---|
| NO: | protein | Sequence |
| 175 | Human | GDYKDDDDKSDKIIHLTDDSFDTDVLKADGAILVDFWAE |
| | osteopontin | WCGPCKMIAPILDEIADEYQGKLTVAKLNIDONPGTAPK |
| | (SPP1(G158- | YGIRGIPTLLLFKNGEVAATKVGALSKGQLKEFLDANLA |
| | N314)) | GRGDSVVYGLRSKSKKFRRPDIQYPDATDEDITSHMESEE |
| | | LNGAYKAIPVAQDLNAPSDWDSRGKDSYETSQLDDQSAE |
| | | THSHKQSRLYKRKANDESNEHSDVIDSQELSKVSREFHSH |
| | | EFHSHEDMLVVDPKSKEEDKHLKFRISHELDSASSEVNVS |
| | | GWRLFKKIS |
| 176 | Ovalbumin | GDYKDDDDKGSIGAASMEFCFDVFKELKVHHANENIFYC |
| | (SERPINB14) | PIAIMSALAMVYLGAKDSTRTQINKVVRFDKLPGFGDSIE |
| | | AQCGTSVNVHSSLRDILNQITKPNDVYSFSLASRLYAEER |
| | | YPILPEYLQCVKELYRGGLEPINFQTAADQARELINSWVE |
| | | SQTNGIIRNVLQPSSVDSQTAMVLVNAIVFKGLWEKAFK |
| | | DEDTQAMPFRVTEQESKPVQMMYQIGLFRVASMASEKM |
| | | KILELPFASGTMSMLVLLPDEVSGLEQLESIINFEKLTEW |
| | | TSSNVMEERKIKVYLPRMKMEEKYNLTSVLMAMGITDV |
| | | FSSSANLSGISSAESLKISQAVHAAHAEINEAGREVVGSAE |
| | | AGVDAASVSEEFRADHPFLFCIKHIATNAVLFFGRCVSPV |
| | | SGWRLFKKIS |
| 177 | Bovine αS1 | RPKHPIKHQGLPQEVLNENLLRFFVAPFPEVFGKEKVNE |
| | casein | LSKDIGSESTEDQAMEDIKQMEAESISSSEEIVPNSVEQKH |
| | (variant C) | IQKEDVPSERYLGYLEQLLRLKKYKVPQLEIVPNSAEERL |
| | (no signal | HSMKEGIHAQQKEPMIGVNQELAYFYPELFRQFYQLDA |
| | peptide) | YPSGAWYYVPLGTQYTDAPSFSDIPNPIGSENSGKTTMPL |
| | | W |
| 416 | Bovine αS1 | MKLLILTCLVAVALARPKHPIKHQGLPQEVLNENLLRFF |
| | casein | VAPFPEVFGKEKVNELSKDIGSESTEDQAMEDIKQMEAE |
| | (variant C) | SISSSEEIVPNSVEQKHIQKEDVPSERYLGYLEQLLRLKKY |
| | with native | KVPQLEIVPNSAEERLHSMKEGIHAQQKEPMIGVNQELA |
| | signal peptide | YFYPELFRQFYQLDAYPSGAWYYVPLGTQYTDAPSFSDIP |
| | | NPIGSENSGKTTMPLW |
| 422 | Bovine αS1 | MRQVWFSWIVGLFLCFFNVSSAAPVNTTTEDETAQIPAE |
| | casein | AVIGYSDLEGDFDVAVLPFSNSTNNGLLFINTTIASIAAKE |
| | (variant C) | EGVSLDKRRPKHPIKHQGLPQEVLNENLLRFFVAPFPEVF |
| | with an N- | GKEKVNELSKDIGSESTEDQAMEDIKQMEAESISSSEEIVP |
| | terminal | NSVEQKHIQKEDVPSERYLGYLEQLLRLKKYKVPQLEIV |
| | alpha mating | PNSAEERLHSMKEGIHAQQKEPMIGVNQELAYFYPELFR |
| | factor (α) | QFYQLDAYPSGAWYYVPLGTQYTDAPSFSDIPNPIGSENS |
| | secretion | GKTTMPLW |
| | signal | |
| 178 | Bovine αS2 | KNTMEHVSSSEESIISQETYKQEKNMAINPSKENLCSTFC |
| | casein (no | KEVVRNANEEEYSIGSSSEESAEVATEEVKITVDDKHYQK |
| | signal | ALNEINQFYQKFPQYLQYLYQGPIVLNPWDQVKRNAVPI |
| | peptide) | TPTLNREQLSTSEENSKKTVDMESTEVFTKKTKLTEEEK |
| | | NRLNFLKKISQRYQKFALPQYLKTVYQHQKAMKPWIQP |
| | | KTKVIPYVRYL |
| 417 | Bovine αS2 | MKFFIFTCLLAVALAKNTMEHVSSSEESIISQETYKQEKN |
| | casein with | MAINPSKENLCSTFCKEVVRNANEEEYSIGSSSEESAEVAT |
| | native signal | EEVKITVDDKHYQKALNEINQFYQKFPQYLQYLYQGPIV |
| | peptide | LNPWDQVKRNAVPITPTLNREQLSTSEENSKKTVDMEST |
| | | EVFTKKTKLTEEEKNRLNFLKKISQRYQKFALPQYLKTV |
| | | YQHQKAMKPWIQPKTKVIPYVRYL |
| 423 | Bovine αS2 | MRQVWFSWIVGLFLCFFNVSSAAPVNTTTEDETAQIPAE |
| | casein with an | AVIGYSDLEGDFDVAVLPFSNSTNNGLLFINTTIASIAAKE |
| | N-terminal | EGVSLDKRKNTMEHVSSSEESIISQETYKQEKNMAINPSK |
| | alpha mating | ENLCSTFCKEVVRNANEEEYSIGSSSEESAEVATEEVKITV |
| | factor (α) | DDKHYQKALNEINQFYQKFPQYLQYLYQGPIVLNPWDQ |
| | secretion | VKRNAVPITPTLNREQLSTSEENSKKTVDMESTEVFTKKT |
| | signal | KLTEEEKNRLNFLKKISQRYQKFALPQYLKTVYQHQKA |
| | | MKPWIQPKTKVIPYVRYL |
| 179 | Bovine β | RELEELNVPGEIVESLSSSEESITRINKKIEKFQSEEQQQTE |
| | casein | DELQDKIHPFAQTQSLVYPFPGPIPNSLPQNIPPLTQTPVV |
| | (variant A2) | VPPFLQPEVMGVSKVKEAMAPKHKEMPFPKYPVEPFTES |
| | (no signal | QSLTLTDVENLHLPLPLLQSWMHQPHQPLPPTVMFPPQS |
| | peptide) | VLSLSQSKVLPVPQKAVPYPQRDMPIQAFLLYQEPVLGP |
| | | VRGPFPIIV |
| 418 | Bovine β | MKVLILACLVALALARELEELNVPGEIVESLSSSEESITRI |
| | casein | NKKIEKFQSEEQQQTEDELQDKIHPFAQTQSLVYPFPGPI |
| | (variant A2) | PNSLPQNIPPLTQTPVVVPPFLQPEVMGVSKVKEAMAPK |
| | with native | HKEMPFPKYPVEPFTESQSLTLTDVENLHLPLPLLQSWM |
| | signal peptide | HQPHQPLPPTVMFPPQSVLSLSQSKVLPVPQKAVPYPQR |
| | | DMPIQAFLLYQEPVLGPVRGPFPIIV |
| 424 | Bovine β | MRQVWFSWIVGLFLCFFNVSSAAPVNTTTEDETAQIPAE |
| | casein | AVIGYSDLEGDFDVAVLPFSNSTNNGLLFINTTIASIAAKE |
| | (variant A2) | EGVSLDKRRELEELNVPGEIVESLSSSEESITRINKKIEKFQ |
| | with an N- | SEEQQQTEDELQDKIHPFAQTQSLVYPFPGPIPNSLPQNIP |
| | terminal | PLTQTPVVVPPFLQPEVMGVSKVKEAMAPKHKEMPFPK |
| | alpha mating | YPVEPFTESQSLTLTDVENLHLPLPLLQSWMHQPHQPLP |
| | factor (a) | PTVMFPPQSVLSLSQSKVLPVPQKAVPYPQRDMPIQAFLL |
| | secretion | YQEPVLGPVRGPFPIIV |
| | signal | |
| 180 | Bovine κ | QEQNQEQPIRCEKDERFFSDKIAKYIPIQYVLSRYPSYGLN |
| | casein | YYQQKPVALINNQFLPYPYYAKPAAVRSPAQILQWQVLS |
| | (variant B) | NTVPAKSCQAQPTTMARHPHPHLSFMAIPPKKNQDKTEI |
| | (no signal | PTINTIASGEPTSTPTIEAVESTVATLEASPEVIESPPEINTV |
| | peptide) | QVTSTAV |
| 419 | Bovine κ | MMKSFFLVVTILALTLPFLGAQEQNQEQPIRCEKDERFFS |
| | casein | DKIAKYIPIQYVLSRYPSYGLNYYQQKPVALINNQFLPYP |
| | (variant B) | YYAKPAAVRSPAQILQWQVLSNTVPAKSCQAQPTTMAR |
| | with native | HPHPHLSFMAIPPKKNQDKTEIPTINTIASGEPTSTPTIEAV |
| | signal peptide | ESTVATLEASPEVIESPPEINTVQVTSTAV |
| 425 | Bovine κ | MRQVWFSWIVGLFLCFFNVSSAAPVNTTTEDETAQIPAE |
| | casein | AVIGYSDLEGDFDVAVLPFSNSTNNGLLFINTTIASIAAKE |
| | (variant B) | EGVSLDKRQEQNQEQPIRCEKDERFFSDKIAKYIPIQYVL |
| | with an N- | SRYPSYGLNYYQQKPVALINNQFLPYPYYAKPAAVRSPA |
| | terminal | QILQWQVLSNTVPAKSCQAQPTTMARHPHPHLSFMAIPP |
| | alpha mating | KKNQDKTEIPTINTIASGEPTSTPTIEAVESTVATLEASPEV |
| | factor (a) | IESPPEINTVQVTSTAV |
| | secretion | |
| | signal | |
| 4 | Bovine αS1 | RPKHPIKHQGLPQEVLNENLLRFFVAPFPEVFGKEKVNE |
| | casein | LSKDIGSESTEDQAMEDIKQMEAESISSSEEIVPNSVEQKH |
| | (variant B) | IQKEDVPSERYLGYLEQLLRLKKYKVPQLEIVPNSAEERL |
| | (signal | HSMKEGIHAQQKEPMIGVNQELAYFYPELFRQFYQLDA |
| | peptide | YPSGAWYYVPLGTQYTDAPSFSDIPNPIGSENSEKTTMPL |
| | removed) | W |
| 420 | Bovine αS1 | MKLLILTCLVAVALARPKHPIKHQGLPQEVLNENLLRFF |
| | casein | VAPFPEVFGKEKVNELSKDIGSESTEDQAMEDIKQMEAE |
| | (variant B) | SISSSEEIVPNSVEQKHIQKEDVPSERYLGYLEQLLRLKKY |
| | with signal | KVPQLEIVPNSAEERLHSMKEGIHAQQKEPMIGVNQELA |
| | peptide | YFYPELFRQFYQLDAYPSGAWYYVPLGTQYTDAPSFSDIP |
| | | NPIGSENSEKTTMPLW |
| 5 | Human | IPVKQADSGSSEEKQLYNKYPDAVATWLNPDPSQKQNLL |
| | osteopontin | APQNAVSSEETNDFKQETLPSKSNESHDHMDDMDDEDDD |
| | (signal | DHVDSQDSIDSNDSDDVDDTDDSHQSDESHHSDESDELVT |
| | peptide | DFPTDLPATEVFTPVVPTVDTYDGRGDSVVYGLRSKSKK |
| | removed) | FRRPDIQYPDATDEDITSHMESEELNGAYKAIPVAQDLNA |
| | | PSDWDSRGKDSYETSQLDDQSAETHSHKQSRLYKRKAND |
| | | ESNEHSDVIDSQELSKVSREFHSHEFHSHEDMLVVDPKSK |
| | | EEDKHLKFRISHELDSASSEVN |
| 421 | Human | MRIAVICFCLLGITCAIPVKQADSGSSEEKQLYNKYPDAV |
| | osteopontin | ATWLNPDPSQKQNLLAPQNAVSSEETNDFKQETLPSKSN |
| | with signal | ESHDHMDDMDDEDDDDHVDSQDSIDSNDSDDVDDTDDSH |
| | peptide | QSDESHHSDESDELVTDFPTDLPATEVFTPVVPTVDTYDG |
| | | RGDSVVYGLRSKSKKFRRPDIQYPDATDEDITSHMESEEL |
| | | NGAYKAIPVAQDLNAPSDWDSRGKDSYETSQLDDQSAET |
| | | HSHKQSRLYKRKANDESNEHSDVIDSQELSKVSREFHSHE |
| | | FHSHEDMLVVDPKSKEEDKHLKFRISHELDSASSEVN |
| 435 | Human | MRFPSIFTAVLFAASSALAAPVNTTTEDETAQIPAEAVIGY |
| | osteopontin | SDLEGDFDVAVLPFSNSTNNGLLFINTTIASIAAKEEGVSL |
| | with N- | EKRIPVKQADSGSSEEKQLYNKYPDAVATWLNPDPSQKQ |
| | terminal α- | NLLAPQNAVSSEETNDFKQETLPSKSNESHDHMDDMDDE |
| | mating factor | DDDDHVDSQDSIDSNDSDDVDDTDDSHQSDESHHSDESDE |
| | secretion | LVTDFPTDLPATEVFTPVVPTVDTYDGRGDSVVYGLRSKS |
| | signal | KKFRRPDIQYPDATDEDITSHMESEELNGAYKAIPVAQDL |
| | | NAPSDWDSRGKDSYETSQLDDQSAETHSHKQSRLYKRKA |
| | | NDESNEHSDVIDSQELSKVSREFHSHEFHSHEDMLVVDPK |
| | | SKEEDKHLKFRISHELDSASSEVN |

### Polynucleotide Expression

Kinases and heterologous protein expression by recombinant host cells described herein may be regulated by a variety of polynucleotide cassette formats. Kinase and heterologous polynucleotide expression may each be regulated by individual promoters. In some embodiments, kinase expression is under the control of a first promoter, and heterologous protein expression (e.g., of a protein that is susceptible to phosphorylation by the kinase) is under the control of a second promoter. In some embodiments, the first promoter is an inducible promoter (e.g., pAOX1) or a constitutive promoter (e.g., pGAP). In some embodiments, the second promoter is an inducible promoter (e.g., pAOX1) or a constitutive promoter (e.g., pGAP). In some embodiments the first promoter and the second promoter are the same. In some embodiments, DNA fragments encoding for the kinase and/or heterologous protein may be site-specifically integrated into the recombinant host cell genome such that the kinase and/or heterologous protein is attached to a native promoter and secretion signal.

Table 12 shows codon optimized nucleic acid sequences encoding 1) bovine αS-1 casein, αS-2 casein, β-casein and κ-casein proteins lacking native signal sequences and having an N-terminal alpha mating factor (α) secretion signal (SEQ ID NO: 426) and 2) encoding bovine αS-1 casein, αS-2 casein, β-casein and κ-casein proteins lacking native signal sequences and lacking an N-terminal alpha mating factor (α) secretion signal (SEQ ID NO: 426). A nucleic acid molecule can comprise, consist, or consist essentially of a sequence having at least 75%, 80%, 85%, 90%, 95%, 96%, 97%, 98%, 99%, or 100% sequence identity to the sequence set forth in any one of SEQ ID NOs: 431, 427, 432, 428, 433, 429, 434, or 430.

TABLE 12
Sequences encoding caseins

| SEQ ID | | |
|---|---|---|
| NO: | Description | Sequence |
| 431 | Codon-optimized | CGACCAAAACATCCTATCAAGCATCAAGGATTGCCTC |
| | sequence encoding | AAGAAGTCTTGAACGAAAATTTACTCAGGTTTTTTGT |
| | bovine αS1 casein | GGCACCTTTTCCAGAAGTCTTTGGAAAGGAGAAGGTC |
| | (variant C) lacking | AATGAACTGTCCAAGGACATTGGTAGTGAATCAACTG |
| | native signal | AGGATCAAGCAATGGAAGATATTAAGCAAATGGAAG |
| | sequence | CTGAAAGCATTTCGTCATCCGAGGAAATTGTTCCCAA |
| | | TAGCGTTGAGCAGAAGCACATACAAAAGGAGGATGT |
| | | ACCGTCTGAGCGTTACCTGGGTTATCTGGAGCAGCTT |
| | | CTCAGATTGAAAAAATACAAAGTACCCCAGTTGGAA |
| | | ATTGTTCCCAACTCTGCTGAGGAAAGATTGCACTCCA |
| | | TGAAAGAGGGAATCCATGCGCAACAGAAAGAACCTA |
| | | TGATAGGTGTGAACCAGGAACTTGCCTACTTCTATCC |
| | | TGAGCTTTTCAGACAATTCTACCAACTGGATGCCTAT |
| | | CCATCTGGTGCATGGTATTACGTTCCACTAGGGACAC |
| | | AATATACCGACGCCCCATCATTCTCTGACATCCCAAA |
| | | TCCTATTGGCTCTGAGAACAGTGGTAAGACTACGATG |
| | | CCATTGTGGTAA |
| 427 | Codon-optimized | ATGAGGCAGGTTTGGTTCTCTTGGATTGTGGGATTGTT |
| | sequence encoding | CCTATGTTTTTTCAACGTGTCTTCTGCTGCTCCAGTCA |
| | bovine αS1 casein | ACACTACAACAGAAGATGAAACGGCACAAATTCCGG |
| | (variant C) with an | CTGAAGCTGTCATCGGTTACTCAGATTTAGAAGGGGA |
| | N-terminal alpha | TTTCGATGTTGCTGTTTTGCCATTTTCCAACAGCACAA |
| | mating factor (a) | ATAACGGGTTATTGTTTATAAATACTACTATTGCCAG |
| | secretion signal | CATTGCTGCTAAAGAAGAAGGGGTATCTTTGGATAAA |
| | | AGACGACCAAAACATCCTATCAAGCATCAAGGATTG |
| | | CCTCAAGAAGTCTTGAACGAAAATTTACTCAGGTTTT |
| | | TTGTGGCACCTTTTCCAGAAGTCTTTGGAAAGGAGAA |
| | | GGTCAATGAACTGTCCAAGGACATTGGTAGTGAATCA |
| | | ACTGAGGATCAAGCAATGGAAGATATTAAGCAAATG |
| | | GAAGCTGAAAGCATTTCGTCATCCGAGGAAATTGTTC |
| | | CCAATAGCGTTGAGCAGAAGCACATACAAAAGGAGG |
| | | ATGTACCGTCTGAGCGTTACCTGGGTTATCTGGAGCA |
| | | GCTTCTCAGATTGAAAAAATACAAAGTACCCCAGTTG |
| | | GAAATTGTTCCCAACTCTGCTGAGGAAAGATTGCACT |
| | | CCATGAAAGAGGGAATCCATGCGCAACAGAAAGAAC |
| | | CTATGATAGGTGTGAACCAGGAACTTGCCTACTTCTA |
| | | TCCTGAGCTTTTCAGACAATTCTACCAACTGGATGCC |
| | | TATCCATCTGGTGCATGGTATTACGTTCCACTAGGGA |
| | | CACAATATACCGACGCCCCATCATTCTCTGACATCCC |
| | | AAATCCTATTGGCTCTGAGAACAGTGGTAAGACTACG |
| | | ATGCCATTGTGGTAA |
| 432 | Codon-optimized | AAGAACACGATGGAACATGTCTCCTCTAGTGAGGAGT |
| | sequence encoding | CTATAATCTCGCAAGAGACATACAAGCAGGAGAAGA |
| | bovine αS2 casein | ATATGGCCATTAATCCAAGCAAGGAGAACTTGTGTTC |
| | (no signal peptide) | CACCTTCTGCAAGGAAGTCGTAAGGAACGCAAATGA |
| | lacking native signal | AGAGGAATACTCTATCGGATCATCTAGTGAGGAATCT |
| | sequence | GCTGAGGTTGCCACTGAGGAAGTCAAGATTACTGTGG |
| | | ACGATAAGCACTACCAAAAAGCTCTAAACGAAATCA |
| | | ATCAGTTTTATCAAAAGTTTCCCCAATATTTGCAATAT |
| | | CTGTACCAAGGTCCAATTGTTTTGAACCCTTGGGATC |
| | | AGGTTAAAAGAAATGCTGTTCCGATTACTCCAACTTT |
| | | AAACAGAGAGCAACTCTCCACCAGTGAGGAAAATTC |
| | | AAAAAAAACCGTGGACATGGAATCAACAGAAGTATT |
| | | CACTAAGAAAACAAAACTGACGGAAGAAGAAAAGA |
| | | ACAGACTAAATTTTTTAAAAAAAATCAGCCAACGTTA |
| | | CCAGAAATTTGCGTTGCCACAGTATCTGAAAACTGTC |
| | | TATCAGCATCAAAAAGCTATGAAGCCTTGGATTCAAC |
| | | CTAAGACAAAGGTTATTCCCTATGTGCGATACCTTTA |
| | | A |
| 428 | Codon-optimized | ATGAGGCAGGTTTGGTTCTCTTGGATTGTGGGATTGTT |
| | sequence encoding | CCTATGTTTTTTCAACGTGTCTTCTGCTGCTCCAGTCA |
| | bovine αS2 casein | ACACTACAACAGAAGATGAAACGGCACAAATTCCGG |
| | with an N-terminal | CTGAAGCTGTCATCGGTTACTCAGATTTAGAAGGGGA |
| | alpha mating factor | TTTCGATGTTGCTGTTTTGCCATTTTCCAACAGCACAA |
| | (a) secretion signal | ATAACGGGTTATTGTTTATAAATACTACTATTGCCAG |
| | | CATTGCTGCTAAAGAAGAAGGGGTATCTTTGGATAAA |
| | | AGAAAGAACACGATGGAACATGTCTCCTCTAGTGAG |
| | | GAGTCTATAATCTCGCAAGAGACATACAAGCAGGAG |
| | | AAGAATATGGCCATTAATCCAAGCAAGGAGAACTTG |
| | | TGTTCCACCTTCTGCAAGGAAGTCGTAAGGAACGCAA |
| | | ATGAAGAGGAATACTCTATCGGATCATCTAGTGAGGA |
| | | ATCTGCTGAGGTTGCCACTGAGGAAGTCAAGATTACT |
| | | GTGGACGATAAGCACTACCAAAAAGCTCTAAACGAA |
| | | ATCAATCAGTTTTATCAAAAGTTTCCCCAATATTTGCA |
| | | ATATCTGTACCAAGGTCCAATTGTTTTGAACCCTTGG |
| | | GATCAGGTTAAAAGAAATGCTGTTCCGATTACTCCAA |
| | | CTTTAAACAGAGAGCAACTCTCCACCAGTGAGGAAA |
| | | ATTCAAAAAAAACCGTGGACATGGAATCAACAGAAG |
| | | TATTCACTAAGAAAACAAAACTGACGGAAGAAGAAA |
| | | AGAACAGACTAAATTTTTTAAAAAAAATCAGCCAAC |
| | | GTTACCAGAAATTTGCGTTGCCACAGTATCTGAAAAC |
| | | TGTCTATCAGCATCAAAAAGCTATGAAGCCTTGGATT |
| | | CAACCTAAGACAAAGGTTATTCCCTATGTGCGATACC |
| | | TTTAA |
| 433 | Codon-optimized | AGAGAGTTGGAAGAATTAAATGTACCAGGTGAGATA |
| | sequence encoding | GTTGAATCTCTATCAAGCTCGGAGGAATCAATTACCC |
| | bovine β casein | GTATAAACAAGAAAATTGAGAAGTTTCAGAGTGAGG |
| | (variant A2) lacking | AACAACAGCAAACAGAGGACGAATTGCAAGATAAAA |
| | native signal | TCCATCCATTTGCGCAAACACAGTCTCTAGTCTATCCC |
| | sequence | TTCCCTGGACCGATCCCTAACTCACTCCCACAAAACA |
| | | TTCCTCCTTTAACTCAAACCCCTGTGGTTGTTCCGCCA |
| | | TTCTTGCAACCAGAAGTAATGGGAGTTTCCAAAGTTA |
| | | AGGAGGCTATGGCTCCTAAGCATAAGGAAATGCCCTT |
| | | CCCAAAATATCCAGTTGAGCCCTTTACTGAAAGCCAG |
| | | TCTCTGACTCTTACGGACGTTGAAAATTTGCACCTTCC |
| | | ACTACCATTGTTGCAGTCTTGGATGCATCAACCTCAC |
| | | CAACCTTTACCACCAACTGTCATGTTTCCTCCTCAATC |
| | | CGTGTTGAGTCTTTCTCAGTCCAAAGTCTTGCCTGTTC |
| | | CGCAGAAAGCTGTGCCATACCCACAGAGAGATATGC |
| | | CCATTCAAGCATTTTTGCTGTACCAAGAACCAGTATT |
| | | AGGTCCAGTTAGGGGCCCCTTCCCTATTATTGTCTAA |
| 429 | Codon-optimized | ATGAGGCAGGTTTGGTTCTCTTGGATTGTGGGATTGTT |
| | sequence encoding | CCTATGTTTTTTCAACGTGTCTTCTGCTGCTCCAGTCA |
| | bovine β casein | ACACTACAACAGAAGATGAAACGGCACAAATTCCGG |
| | (variant A2) with an | CTGAAGCTGTCATCGGTTACTCAGATTTAGAAGGGGA |
| | N-terminal alpha | TTTCGATGTTGCTGTTTTGCCATTTTCCAACAGCACAA |
| | mating factor (α) | ATAACGGGTTATTGTTTATAAATACTACTATTGCCAG |
| | secretion signal | CATTGCTGCTAAAGAAGAAGGGGTATCTTTGGATAAA |
| | | AGAAGAGAGTTGGAAGAATTAAATGTACCAGGTGAG |
| | | ATAGTTGAATCTCTATCAAGCTCGGAGGAATCAATTA |
| | | CCCGTATAAACAAGAAAATTGAGAAGTTTCAGAGTG |
| | | AGGAACAACAGCAAACAGAGGACGAATTGCAAGATA |
| | | AAATCCATCCATTTGCGCAAACACAGTCTCTAGTCTA |
| | | TCCCTTCCCTGGACCGATCCCTAACTCACTCCCACAA |
| | | AACATTCCTCCTTTAACTCAAACCCCTGTGGTTGTTCC |
| | | GCCATTCTTGCAACCAGAAGTAATGGGAGTTTCCAAA |
| | | GTTAAGGAGGCTATGGCTCCTAAGCATAAGGAAATG |
| | | CCCTTCCCAAAATATCCAGTTGAGCCCTTTACTGAAA |
| | | GCCAGTCTCTGACTCTTACGGACGTTGAAAATTTGCA |
| | | CCTTCCACTACCATTGTTGCAGTCTTGGATGCATCAAC |
| | | CTCACCAACCTTTACCACCAACTGTCATGTTTCCTCCT |
| | | CAATCCGTGTTGAGTCTTTCTCAGTCCAAAGTCTTGCC |
| | | TGTTCCGCAGAAAGCTGTGCCATACCCACAGAGAGAT |
| | | ATGCCCATTCAAGCATTTTTGCTGTACCAAGAACCAG |
| | | TATTAGGTCCAGTTAGGGGCCCCTTCCCTATTATTGTC |
| | | TAA |
| 434 | Codon-optimized | CAAGAACAAAACCAAGAACAACCCATAAGATGTGAG |
| | sequence encoding | AAGGATGAAAGATTCTTCTCTGACAAAATAGCCAAAT |
| | bovine κ casein | ATATACCAATTCAGTACGTGCTATCTAGGTATCCTAG |
| | (variant B) lacking | CTATGGACTCAATTACTACCAACAGAAACCAGTTGCT |
| | native signal | TTGATTAACAATCAATTTCTTCCATACCCCTATTACGC |
| | sequence | AAAGCCCGCTGCAGTTCGATCCCCTGCGCAAATTCTT |
| | | CAATGGCAAGTCTTGTCAAATACTGTGCCTGCTAAGT |
| | | CCTGCCAAGCTCAGCCGACGACGATGGCTCGTCACCC |
| | | ACATCCACATTTATCTTTTATGGCCATTCCGCCAAAG |
| | | AAAAATCAGGATAAAACAGAAATCCCTACCATCAAC |
| | | ACCATTGCTAGTGGCGAGCCTACATCCACACCTACCA |
| | | TTGAAGCAGTAGAGTCAACTGTAGCTACTTTGGAAGC |
| | | CTCTCCAGAAGTTATTGAGTCGCCACCTGAGATCAAC |
| | | ACAGTCCAGGTTACTTCAACTGCTGTCTAA |
| 430 | Codon-optimized | ATGAGGCAGGTTTGGTTCTCTTGGATTGTGGGATTGTT |
| | sequence encoding | CCTATGTTTTTTCAACGTGTCTTCTGCTGCTCCAGTCA |
| | bovine κ casein | ACACTACAACAGAAGATGAAACGGCACAAATTCCGG |
| | (variant B) with an | CTGAAGCTGTCATCGGTTACTCAGATTTAGAAGGGGA |
| | N-terminal alpha | TTTCGATGTTGCTGTTTTGCCATTTTCCAACAGCACAA |
| | mating factor (α) | ATAACGGGTTATTGTTTATAAATACTACTATTGCCAG |
| | secretion signal | CATTGCTGCTAAAGAAGAAGGGGTATCTTTGGATAAA |
| | | AGACAAGAACAAAACCAAGAACAACCCATAAGATGT |
| | | GAGAAGGATGAAAGATTCTTCTCTGACAAAATAGCC |
| | | AAATATATACCAATTCAGTACGTGCTATCTAGGTATC |
| | | CTAGCTATGGACTCAATTACTACCAACAGAAACCAGT |
| | | TGCTTTGATTAACAATCAATTTCTTCCATACCCCTATT |
| | | ACGCAAAGCCCGCTGCAGTTCGATCCCCTGCGCAAAT |
| | | TCTTCAATGGCAAGTCTTGTCAAATACTGTGCCTGCT |
| | | AAGTCCTGCCAAGCTCAGCCGACGACGATGGCTCGTC |
| | | ACCCACATCCACATTTATCTTTTATGGCCATTCCGCCA |
| | | AAGAAAAATCAGGATAAAACAGAAATCCCTACCATC |
| | | AACACCATTGCTAGTGGCGAGCCTACATCCACACCTA |
| | | CCATTGAAGCAGTAGAGTCAACTGTAGCTACTTTGGA |
| | | AGCCTCTCCAGAAGTTATTGAGTCGCCACCTGAGATC |
| | | AACACAGTCCAGGTTACTTCAACTGCTGTCTAA |

### III. Bioreactors

Bioreactors are described herein for obtaining a phosphorylated secreted protein. The bioreactor may comprise a plurality of recombinant host cells, a reaction vessel (e.g., shake flask), and media (e.g., YPD and BMGY), wherein the media and the recombinant host cells are disposed within the reaction vessel. The plurality of recombinant host cells may be engineered to express a heterologous serine/threonine kinase such that 24 hours after expression of the heterologous serine/threonine kinase, at least 60% of the serine/threonine kinase in the reaction vessel is intact. In some embodiments, the plurality of recombinant host cells in the bioreactor are engineered to further express a heterologous secreted protein comprising one or more phosphorylation sites susceptible to phosphorylation by the serine/threonine kinase. The bioreactor may be maintained at an internal temperature between 20 degrees Celsius and 40 degrees Celsius. The various embodiments of the recombinant host cells described herein may be used as the plurality of recombinant host cells used in the bioreactor system. In some embodiments, at least 70%, 80%, or 90% of the serine/threonine kinase in the reaction vessel is intact 24 hours after expression initiation. In some instances, expression of the serine/threonine kinase is initiated by adding an inducing agent to the media. Inducing agents for the initiation of heterologous serine/threonine kinase expression described herein may include ethanol, maltose, starch, xylose, thiamine, copper, quinic acid, nitrate, glucose, saccharides, H2O2, CaCO3 or benzoic acid. In some instances, expression of the serine/threonine kinase is initiated by light, blue light, low pH, iron starvation or copper depletion. In some instances, expression of the serine/threonine kinase is initiated by culturing the recombinant host cells in the media.

### IV. Methods of Manufacturing a Phosphorylated Protein

A method of manufacturing a phosphorylated heterologous protein, which may be secreted, is described herein. The method comprises expressing, in a cell population comprising a plurality of recombinant host cells, (1) a heterologous serine/threonine kinase and (2) a heterologous secreted protein comprising one or more phosphorylation sites susceptible to phosphorylation by the serine/threonine kinase; and culturing the cell population in a reaction vessel containing media (e.g., YPD and BMGY) for at least 24 hours after expressing the heterologous serine/threonine kinase and the heterologous secreted protein, at least 60% of the serine/threonine kinase in the reaction vessel is intact. In some embodiments, at least 70%, 80%, or 90% of the serine/threonine kinase in the reaction vessel is intact 24 hours after expression initiation. In some instances, the heterologous serine/threonine kinase expressed by recombinant host cells may be of the modified form as described herein, such as the incorporation of an intracellular membrane-anchoring domain. In some embodiments, the expression of the heterologous serine/threonine kinase may comprise culturing the recombinant host cells in the media. In some embodiments, the expression of heterologous serine/threonine kinase comprises adding an induction agent to the media, wherein the induction agent may be methanol, IPTG, ethanol, maltose, starch, xylose, thiamine, copper, quinic acid, nitrate, glucose, saccharides, H2O2, CaCO3, or benzoic acid. In some embodiments, the expression of heterologous serine/threonine kinase may comprise induction by light, blue light, low pH, iron starvation or copper depletion. In some embodiments, the method further comprises culturing the plurality of recombinant cells at a temperature between 20 degrees Celsius and 40 degrees Celsius. In some embodiments, the method further comprises harvesting the phosphorylated protein by centrifuging the plurality of recombinant host cells and collecting the supernatant.

### Generating Recombinant Host Cells

Recombinant host cells described in the method may further comprise additional procedures described herein to express the serine/threonine kinase and heterologous protein of interest. Recombinant host cells may be generated by transforming the cell population with polynucleotide expression cassettes encoding the serine/threonine kinase under control of a first promoter and the secreted protein comprising one or more phosphorylation sites susceptible to phosphorylation by the serine/threonine kinase under control of a second promoter. In some instances, the recombinant cell population may be transformed sequentially with (1) a first vector encoding the serine/threonine kinase under control of a first promoter and with (2) a second vector encoding the secreted protein comprising one or more phosphorylation sites susceptible to phosphorylation by the serine/threonine kinase under control of a second promoter. In some instances, the recombinant cell population may be transformed with a vector encoding both (1) the serine/threonine kinase under control of a first promoter and (2) the secreted protein comprising one or more phosphorylation sites susceptible to phosphorylation by the serine/threonine kinase under control of a second promoter.

In some instances, distinct fragments of DNA encoding (1) a serine/threonine kinase under control of a first promoter and (2) a secreted protein comprising one or more phosphorylation sites susceptible to phosphorylation by the serine/threonine kinase under control of a second promoter may be incorporated into the recombinant host cell genome. In some instances, a fragment of DNA encoding both (1) the serine/threonine kinase under control of a first promoter and (2) the secreted protein comprising one or more phosphorylation sites susceptible to phosphorylation by the serine/threonine kinase under control of a second promoter may be incorporated into the recombinant host cell genome.

### Phosphorylated Protein Products

The phosphorylated protein made by the method described herein, which may be made by use of a heterologous serine/threonine kinase or a modified serine/threonine kinase, may be used to generate one or more therapeutic and/or nutritional products. Stated differently, the therapeutic and/or nutritional products may comprise the phosphorylated protein made by the method described herein. In some embodiments, the phosphorylated protein made by the method described herein may be used in a food product. In some instances, the food product is a dairy product, a dairy substitute, or cheese. In some embodiments, the phosphorylated protein made by the method described herein may also be used in a nutritional supplement or a therapeutic composition comprising a therapeutically effective amount of the phosphorylated protein.

In some embodiments, the phosphorylated protein can have a distinct phosphorylation pattern relative to the phosphorylated protein in its native state (i.e. as found in nature). In some embodiments, the phosphorylated protein can be more phosphorylated relative to the phosphorylated protein in its native state. In some embodiments, the phosphorylated protein can be less phosphorylated relative to the phosphorylated protein in its native state. In some embodiments, the phosphorylated protein can have an increased number of phosphorylation sites relative to the phosphorylated protein in its native state. In some embodiments, the phosphorylated protein can have a decreased number of phosphorylation sites relative to the phosphorylated protein in its native state. In some embodiments, the phosphorylated protein can have an increased abundance of phosphorylation at a particular phosphorylation site relative to the phosphorylated protein in its native state. In some embodiments, the phosphorylated protein can have a decreased abundance of phosphorylation at a particular phosphorylation site relative to the phosphorylated protein in its native state.

In some embodiments, the phosphorylated protein has at least one more phosphorylated site than the phosphorylated protein in its native state. In some embodiments, the phosphorylated protein has at least two, at least three, at least five, at least ten, at least fifteen, or at least twenty more phosphorylated sites than the phosphorylated protein in its native state.

In some embodiments, the phosphorylated protein has at least one fewer phosphorylated sites than the phosphorylated protein in its native state. In some embodiments, the phosphorylated protein has at least two, at least three, at least five, at least ten, at least fifteen, or at least twenty fewer phosphorylated sites than the phosphorylated protein in its native state.

In some embodiments, the phosphorylated protein may be made by use of a heterologous serine/threonine kinase. In some embodiments, the phosphorylated protein may be made by use of a modified heterologous serine/threonine kinase. In some embodiments, the phosphorylated protein made by use of a modified heterologous serine/threonine kinase can have a distinct phosphorylation pattern relative to the unmodified heterologous serine/threonine kinase. In some embodiments, the phosphorylated protein made by use of a modified heterologous serine/threonine kinase can be more phosphorylated relative to the unmodified heterologous serine/threonine kinase. In some embodiments, the phosphorylated protein made by use of a modified heterologous serine/threonine kinase can be less phosphorylated relative to the unmodified heterologous serine/threonine kinase. In some embodiments, the phosphorylated protein made by use of a modified heterologous serine/threonine kinase can have an increased number of phosphorylation sites relative to the unmodified heterologous serine/threonine kinase. In some embodiments, the phosphorylated protein made by use of a modified heterologous serine/threonine kinase can have a decreased number of phosphorylation sites relative to the unmodified heterologous serine/threonine kinase. In some embodiments, the phosphorylated protein made by use of a modified heterologous serine/threonine kinase can have an increased abundance of phosphorylation at a particular phosphorylation site relative to the unmodified heterologous serine/threonine kinase. In some embodiments, the phosphorylated protein made by use of a modified heterologous serine/threonine kinase can have a decreased abundance of phosphorylation at a particular phosphorylation site relative to the unmodified heterologous serine/threonine kinase.

In some embodiments, the phosphorylated protein can be a therapeutic protein or a nutritional protein. In some embodiments, the nutritional protein can be a dairy protein. In some embodiments, the dairy protein can be a casein or a portion thereof. In some embodiments, the casein can be αs1-casein, αs2-casein, β-casein, or κ-casein or a portion thereof. In some embodiments, the dairy protein can be glycomacropeptide or a portion thereof. In some embodiments, the dairy protein can be osteopontin or a portion thereof. In some embodiments, the dairy protein can be lactoferrin or a portion thereof. In some embodiments, the nutritional protein can be an egg white protein. In some embodiments, the egg white protein can be ovalbumin or a portion thereof.

In some embodiments, the phosphorylated protein can be ovalbumin. In some embodiments, the ovalbumin can be phosphorylated at residues comprising S69, T76, T92, S99, S165, T202, S206, and S345. In some embodiments, the ovalbumin can be phosphorylated at residues comprising T92, S99, S165, T202, S206, S222, S271, and S345.

In some embodiments, the phosphorylated protein can be casein or a portion thereof. In some embodiments, the casein can be αs1-casein, αs2-casein, β-casein, or κ-casein, or a portion of any thereof.

In some embodiments, the casein is an αS1-casein (e.g., bovine αS-1 casein (variant C)). The αS1-casein (e.g., bovine αS-1 casein (variant C)) can comprise, consist, or consist essentially of the amino acid sequence set forth in SEQ ID NO: 416 or a fragment of full-length &S1-casein (e.g., bovine αS-1 casein (variant C)), e.g., the sequence set forth in SEQ ID NO: 177; the αS1-casein (e.g., bovine αS-1 casein (variant C)) can be coupled to an N-terminal alpha mating factor (α) secretion signal and can comprise, consist, or consist essentially of the amino acid sequence the sequence set forth in SEQ ID NO: 422. In some embodiments, the αS1-casein (e.g., bovine αS-1 casein (variant C)) is phosphorylated at one or more, two or more, three or more, four or more, five or more, six or more, seven or more, eight or more, nine or more, ten or more, eleven or more, twelve or more, fifteen or more, or twenty or more amino acids (e.g., serines and/or threonines). In some embodiments, the αS1-casein (e.g., bovine αS-1 casein (variant C)) is phosphorylated at one or more of, two or more of, three or more of, four or more of, five or more of, six or more of, seven or more of, eight or more of, nine or more of, ten or more of, or eleven of S56, S61, S63, T64, S79, S81, S82, S83, S90, S103, or S130 (numbering relative to bovine αS-1 casein (variant C) sequence in SEQ ID NO: 416). In some embodiments, the αS1-casein (e.g., bovine αS-1 casein (variant C)) is phosphorylated at one or more of, two or more of, three of more of, four or more of, or five or more of S56, S79, S81, S82, S83, or S103. In some embodiments, the αS1-casein (e.g., bovine αS-1 casein (variant C)) is phosphorylated at S56. In some embodiments, the αS1-casein (e.g., bovine αS-1 casein (variant C)) is phosphorylated at one or more of, two or more of, three or more of, four or more of, five or more of, six or more of, seven or more of, eight or more of, nine or more of, or ten of S61, S63, T64, S79, S81, S82, S83, S90, S103, or S130. In some embodiments, the αS1-casein (e.g., bovine αS-1 casein (variant C)) is phosphorylated at S61, S63, T64, S79, S81, S82, S83, S90, S103, and S130. In some embodiments, the αS1-casein (e.g., bovine αS-1 casein (variant C)) comprises, consists, or consists essentially of an amino acid sequence with at least 70%, 75%, 80%, 85%, 90%, 95%, 96%, 97%, 98%, 99%, or 100% sequence identity to the amino acid sequence set forth in SEQ ID NO: 177, 416, or 422. In some embodiments, the αS1-casein (e.g., bovine αS-1 casein (variant C)) comprises, consists, or consists essentially of an amino acid sequence of at least 70%, 75%, 80%, 85%, 90%, 95%, 96%, 97%, 98%, 99%, or 100% sequence identity to the amino acid sequence set forth in SEQ ID NO: 177.

In some embodiments, the phosphorylated casein is an αS2-casein (e.g., bovine αS2-casein). The αS2-casein (e.g., bovine αS2-casein) can comprise, consist, or consist essentially of the amino acid sequence set forth in SEQ ID NO: 417 or be a fragment of full-length αS2-casein (e.g., bovine αS2-casein) comprising, consisting, or consisting essentially of, e.g., the amino acid sequence set forth in SEQ ID NO: 178; the αS2-casein (e.g., bovine αS2-casein) can be coupled to an N-terminal alpha mating factor (α) secretion signal and can comprise, consist, or consist essentially of the amino acid sequence set forth in SEQ ID NO: 423. In some embodiments, the αS2-casein (e.g., bovine αS2-casein) is phosphorylated at one or more, two or more, three or more, four or more, five or more, six or more, seven or more, eight or more, nine or more, ten or more, eleven or more, twelve or more, fifteen or more, or twenty or more amino acids (e.g., serines and/or threonines). In some embodiments, the αS2-casein (e.g., bovine αS2-casein) is phosphorylated at one or more of, two or more of, three or more of, four or more of, five or more of, six or more of, seven or more of, eight or more of, nine or more of, 10 or more of, 11 or more of, 12 of more of, 13 or more of, 14 or more of, or 15 or more of S23, S24, S25, S46, S52, S71, S72, S73, S76, T145, S146, S150, S158, T159, or T163. In some embodiments, the αS2-casein (e.g., bovine αS2-casein) is phosphorylated at one or more of, two or more of, three of more of, four or more of, five or more of, six or more of seven or more of, or eight of S23, S24, S25, S52, S71, S72, S73, or S76. In some embodiments, the αS2-casein (e.g., bovine αS2-casein) is phosphorylated at one or more of, two or more of, three of more of, four or more of, five or more of, or six of S23, S52, S71, S72, S73, or S76. In some embodiments, the αS2-casein (e.g., bovine αS2-casein) is phosphorylated at S23, S52, S71, S72, S73, and S76. In some embodiments, the αS2-casein (e.g., bovine αS2-casein) is phosphorylated at S23, S24, S25, S46, S52, S71, S72, S73, S76, T145, S146, S150, S158, and T159. In some embodiments, the αS2-casein (e.g., bovine αS2-casein) comprises, consists, or consists essentially of an amino acid sequence with at least 70%, 75%, 80%, 85%, 90%, 95%, 96%, 97%, 98%, 99%, or 100% sequence identity to the amino acid sequence set forth in SEQ ID NO: 178, 417, or 423. In some embodiments, the αS2-casein (e.g., bovine αS2-casein) comprises, consists, or consists essentially of an amino acid sequence with at least 70%, 75%, 80%, 85%, 90%, 95%, 96%, 97%, 98%, 99%, or 100% sequence identity to the amino acid sequence set forth in SEQ ID NO: 178.

In some embodiments, the phosphorylated casein is a β-casein (e.g., bovine β casein (variant A2)). The β-casein (e.g., bovine β casein (variant A2)) can comprise, consist, or consist essentially of the amino acid sequence set forth in SEQ ID NO: 418 or be a fragment of β-casein (e.g., bovine β casein (variant A2)), e.g., comprising, consisting, or consisting essentially of the amino acid sequence set forth in SEQ ID NO: 179; the β-casein (e.g., bovine β casein (variant A2) can be coupled to an N-terminal alpha mating factor (α) secretion signal and can comprise, consist, or consist essentially of the amino acid sequence set forth in SEQ ID NO: 424. In some embodiments, the β-casein (e.g., bovine β casein (variant A2)) is phosphorylated at one or more of, two or more of, three or more of, four or more of, five or more of, six or more of, seven or more of, or eight of S30, S32, S33, S34, S37, T39, S50, at T56. In some embodiments, the β-casein (e.g., bovine β casein (variant A2)) is phosphorylated at one or more of, two or more of, three or more of, four or more of, or five of S30, S32, S33, S34, S37, T39. In some embodiments, the β-casein (e.g., bovine β casein (variant A2)) is phosphorylated at S37. In some embodiments, the β-casein (e.g., bovine β casein (variant A2)) is phosphorylated at S30, S32, S33, S34, S37, T39, S50, and T56. In some embodiments, the β-casein (e.g., bovine β casein (variant A2) comprises, consists, or consists essentially of an amino acid sequence with at least 70%, 75%, 80%, 85%, 90%, 95%, 96%, 97%, 98%, 99%, or 100% sequence identity to the amino acid sequence set forth in SEQ ID NO: 179, 418, or 424. In some embodiments, the β-casein (e.g., bovine β casein (variant A2) comprises, consists, or consists essentially of an amino acid sequence with at least 70%, 75%, 80%, 85%, 90%, 95%, 96%, 97%, 98%, 99%, or 100% sequence identity to the amino acid sequence set forth in SEQ ID NO: 179.

In some embodiments, the phosphorylated casein is κ-casein (e.g., bovine κ-casein). The κ-casein (e.g., bovine κ-casein) can comprise, consist, or consist essentially of the amino acid sequence set forth in SEQ ID NO: 419, or be a fragment of κ-casein (e.g., bovine κ-casein) comprising, consisting, or consisting essentially of the amino acid sequence set forth in SEQ ID NO: 180; the κ-casein (e.g., bovine κ-casein) can be coupled to an N-terminal alpha mating factor (α) secretion signal and can comprise, consist, or consist essentially of the amino acid sequence set forth in SEQ ID NO: 425. In some embodiments, the κ-casein (e.g., bovine κ-casein) comprises, consists, or consists essentially of an amino acid sequence with at least 70%, 75%, 80%, 85%, 90%, 95%, 96%, 97%, 98%, 99%, or 100% sequence identity to the amino acid sequence set forth in SEQ ID NO: 180, 419, or 425. In some embodiments, the κ-casein (e.g., bovine κ-casein) comprises, consists, or consists essentially of an amino acid sequence with at least 70%, 75%, 80%, 85%, 90%, 95%, 96%, 97%, 98%, 99%, or 100% sequence identity to the amino acid sequence set forth in SEQ ID NO: 180.

In some embodiments, the casein is phosphorylated at one or more amino acid residues that are not phosphorylated in nature. In some embodiments, the casein is phosphorylated at two or more amino acid residues that are not phosphorylated in nature. In some embodiments, the casein is phosphorylated at three or more amino acid residues that are not phosphorylated in nature. In some embodiments, the casein is phosphorylated at four or more amino acid residues that are not phosphorylated in nature. In some embodiments, the casein is phosphorylated at five or more amino acid residues that are not phosphorylated in nature. In some embodiments, the casein is phosphorylated at six or more, seven or more, eight or more, nine or more, ten or more, 11 or more, 12 or more, 13 or more 14 or more, 15 or more, 16 or more, 17 or more, 18 or more, 19 or more, or 20 or more amino acid residues that are not phosphorylated in nature.

### EXAMPLES

The following illustrative examples are representative of embodiments of compositions and methods described herein and are not meant to be limiting in any way.

### Example 1: Expression of Reptile Serine/Threonine Protein Kinase Fam20c in Pichia pastoris Yeast Using a Non-Constitutive Promoter

### Vector Design

For recombinant protein expression in yeast (P. pastoris), recombinant vectors encoding a snake Ser/Thr Fam20c kinase (SEQ ID NO: 3) were generated and codon-optimized for expression in P. pastoris. DNA encoding the Ser/Thr kinases of SEQ ID NO: 3 were cloned into a pD902 vector, which is an integrative vector targeting the AOX1 or GAP locus in P. pastoris. The expression cassette included an N-terminal α-mating factor secretion signal, a FLAG-SUMO tag, and a C-terminal HiBiT tag. In one variant, the expression of the recombinant snake Ser/Thr Fam20c protein was controlled by the pAOX1 promoter, which is a non-constitutive inducible promoter activated by the presence of methanol in the growth media (variant Fam20C Common Garter Snake (FCGS)-3). In a second variant, the expression of the recombinant snake Ser/Thr Fam20c protein was controlled by pGAP, which is a constitutive promoter (variant FCGS-8). In a third variant, the expression of the recombinant snake Ser/Thr Fam20c protein (with an N-terminal FLAG tag) was controlled by the pGAP promoter (variant FCGS-5).

### Expression of Recombinant Snake Ser/Thr Fam20c Protein in Eukaryotic Host Cells

The snake Fam20c vectors were transformed into P. pastoris CBS7435, and putative strains were confirmed by colony PCR. The strains were grown overnight in BMGY (non-induction media), diluted to an OD600 of 1.0 in BMMY (induction media), and expression of recombinant snake Ser/Thr Fam20c proteins were monitored between 0 and 48 hours post-induction. At each 24-hour time point, cultures were supplemented with additional methanol to maintain induction conditions. Cultures were harvested, and the supernatant was retained for analysis.

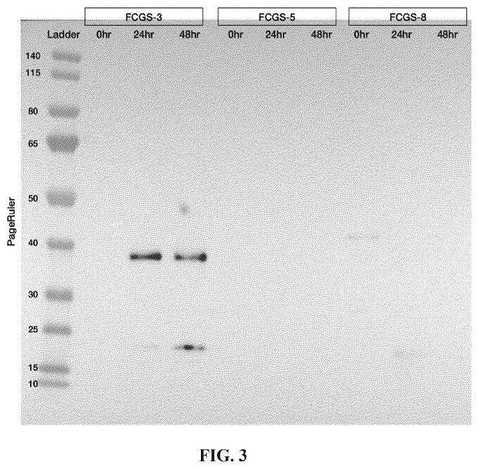

Extracellular expression of snake Ser/Thr Fam20c FCGS-3, FCGS-5, and FCGS-8 variants from P. pastoris CBS7435 was probed during a span of 0 to 48 hours after induction of protein expression at 24-hour time points (FIG. 3). Supernatant samples were separated by denaturing protein gel. The samples were then transferred to a nitrocellulose membrane and probed with an anti-FLAG primary antibody conjugated to HRP.

Shown in FIG. 3 is a western blot of proteins secreted from transgenic P. pastoris CBS7435 expressing the snake Fam20c FCGS-3, FCGS-5, or FCGS-8 variant probed using an anti-FLAG primary antibody conjugated to HRP. The expression time points are indicated at the top of each lane. The expected weight for the FCGS-3 variant is 30.2 kDa (FIG. 3, left). The expected weight for the FCGS-5 variant is 19 kDa (FIG. 3, center). The expected weight for the FCGS-8 variant is 30.2 kDa (FIG. 3, right). Expression in the FCGS-8 variant, in which expression is driven by the constitutive pGAP promoter is reduced from that seen in the FCGS-3 variant, in which expression is driven by the inducible pAOX1 promoter. In FCGS-5, in which the N-terminal SUMO tag is removed, the expression of snake Fam20c is greatly reduced. Taken together, these data indicate that robust expression of snake Fam20c with a solubility enhancer tag can be achieved.

### Example 2: Expression and Stability of Mouse, Bovine and Snake Serine/Threonine Protein Kinase (Fam20c) in Yeast Cells

For recombinant protein expression in yeast (Pichia pastoris (P. pastoris)), recombinant vectors encoding a mouse serine/threonine protein kinase Fam20c (mouse Ser/Thr Fam20c; SEQ ID NO: 1), a bovine Ser/Thr Fam20c kinase (SEQ ID NO: 2), and a snake Ser/Thr Fam20c kinase (SEQ ID NO: 3) were generated and codon-optimized for expression in P. pastoris. DNA encoding the Ser/Thr kinases of SEQ ID NOS: 1, 2, and 3 were cloned into a pD902 vector, which is an integrative vector targeting the AOX1 locus in P. pastoris. The expression cassette included an N-terminal α-mating factor secretion signal, a FLAG-SUMO tag, and a C-terminal protein detection HiBiT tag. The expression of the recombinant Ser/Thr Fam20c protein was controlled by the AOX1 promoter, an inducible promoter activated by the presence of methanol in the growth media.

The resulting mouse, bovine, and snake Fam20c vectors were transformed into P. pastoris CBS7435, and putative recombinant strains were confirmed by colony PCR. The strains were grown overnight in BMGY (non-induction media), diluted to an OD600 of 1.0 in BMMY (induction media), and expression of recombinant Ser/Thr Fam20c proteins were monitored between 0 and 96 hours post-induction. At each 24-hour time point, cultures were supplemented with additional methanol to maintain induction conditions. Cultures were harvested. The cell pellets were lysed by bead beating. The cell lysate and the supernatant were retained for analysis.

Extracellular expression of mouse and bovine Ser/Thr Fam20c and intracellular expression of mouse, bovine, and snake Ser/Thr Fam20c from P. pastoris CBS7435 was probed during a span of 0 to 96 hours after induction of protein expression at 24-hour time points. Samples were separated by denaturing protein gel. The samples were then transferred to a nitrocellulose membrane and probed with an anti-FLAG primary antibody conjugated to HRP.

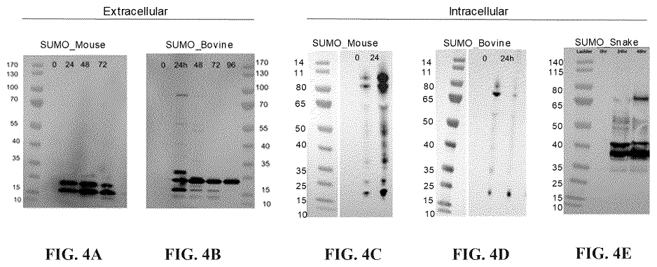

Shown in FIG. 4A and FIG. 4B are western blots of protein extracted from the supernatant portion of transgenic P. pastoris CBS7435 expressing mouse or bovine Fam20c, respectively, probed using an anti-FLAG primary antibody conjugated to HRP. Shown in FIG. 4C, FIG. 4D, and FIG. 4E are western blots of protein extracted from the cell lysate of transgenic P. pastoris CBS7435 expressing mouse, bovine or snake Fam20c, respectively, probed using an anti-FLAG primary antibody conjugated to HRP. The expression time points are indicated at the top of each lane. For mouse Ser/Thr Fam20c, the expected weight for a full-length product is 74.5 kDa. For bovine Ser/Thr Fam20c, the expected weight for a full-length product is 77.8 kDa. For snake Ser/Thr Fam20c, the expected weight for a full-length product is 30.2 kDa. The mouse Fam20c and bovine Fam20c extracted from both the extracellular and intracellular portion of the culture were fragmented (FIG. 4A-D). In contrast, the snake gene construct (FIG. 4E) produced the full-length (intact) serine/threonine kinase Fam20c.

### Example 3: Primate and Avian Serine/Threonine Protein Kinase Fam20c Expression in Pichia pastoris Yeast

### Vector Design for Constitutive Expression of Primate and Avian Serine/Threonine Protein Kinase Fam20c

For recombinant protein expression in yeast (P. pastoris), recombinant vectors encoding a primate serine/threonine protein kinase (lemur Ser/Thr Fam20c, SEQ ID NO: 6) or an avian Ser/Thr Fam20c (sandgrouse Ser/Thr Fam20c; SEQ ID NO: 8) were generated and codon-optimized for expression in P. pastoris. DNA encoding the Ser/Thr kinases of SEQ ID NOS: 6 and 8 (which do not include N-terminal native secretion signals) were cloned into a pD902 vector, which is an integrative vector targeting the GAP locus in P. pastoris. The expression cassette included an N-terminal α-mating factor secretion signal, a FLAG-SUMO tag, and a C-terminal HiBiT tag. In both variants, the expression of the recombinant lemur or sandgrouse Ser/Thr Fam20c protein was controlled by pGAP, which is a constitutive promoter.

Constitutive Expression of Recombinant Ser/Thr Fam20c Proteins in P. pastoris

The vectors were transformed into P. pastoris CBS7435, and putative strains were confirmed by colony PCR. The strains were grown overnight in YPD, diluted to an OD600 of 1.0 in YPD, and expression of recombinant Ser/Thr Fam20c proteins was monitored between 0 and 48 hours post-inoculation. At each 24-hour time point, cultures were supplemented with additional glucose to maintain growth conditions. Cultures were harvested. The cell pellets were lysed by bead beating. The cell lysate and the supernatant were retained for analysis.

Extracellular and intracellular expression of lemur and sandgrouse Ser/Thr Fam20c variants from P. pastoris CBS7435 was probed during a span of 0 to 48 hours post-inoculation at 24-hour time points. Samples were separated by denaturing protein gel. The samples were then transferred to a nitrocellulose membrane and probed with an anti-FLAG primary antibody conjugated to HRP.

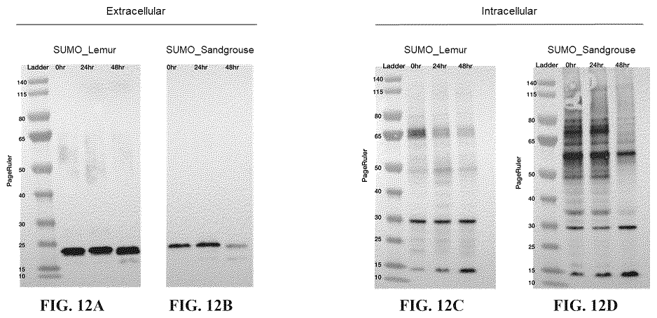

Shown in FIG. 12A and FIG. 12B are western blots of protein extracted from the supernatant portion of transgenic P. pastoris CBS7435 expressing lemur or sandgrouse Fam20c, respectively, probed using an anti-FLAG primary antibody conjugated to HRP. Shown in FIG. 12C and FIG. 12D are western blots of protein extracted from the cell lysate of transgenic P. pastoris CBS7435 expressing lemur or sandgrouse Fam20c, respectively, probed using an anti-FLAG primary antibody conjugated to HRP. The expression time points are indicated at the top of each lane. For lemur Ser/Thr Fam20c, the expected weight for a full-length product is 43.3 kDa. For sandgrouse Ser/Thr Fam20c, the expected weight for a full-length product is 48.6 kDa. The lemur Fam20c and sandgrouse Fam20c extracted from the extracellular portion of the culture were fragmented (FIGS. 12A-B). In contrast, the intracellular portion of the culture produced the full-length (intact) serine/threonine kinase Fam20c for both constructs, lemur and sandgrouse (FIGS. 12C-D).

### Vector Design for Inducible Expression of Primate and Avian Serine/Threonine Protein Kinase Fam20c

For recombinant protein expression in yeast (P. pastoris), recombinant vectors encoding a primate serine/threonine protein kinase (lemur Ser/Thr Fam20c; SEQ ID NO: 6) or an avian Ser/Thr Fam20c (sandgrouse Ser/Thr Fam20c; SEQ ID NO: 8) were generated and codon-optimized for expression in P. pastoris. DNA encoding the Ser/Thr kinases of SEQ ID NOS: 6 and 8 (which do not include N-terminal native secretion signals) were cloned into a pD902 vector, which is an integrative vector targeting the AOX1 locus in P. pastoris. The expression cassette included an N-terminal α-mating factor secretion signal, a FLAG-SUMO tag, and a C-terminal HiBiT tag. The expression of the recombinant Ser/Thr Fam20c protein was controlled by the pAOX1 promoter, which is an inducible promoter activated by the presence of methanol in the growth media.

Inducible Expression of Recombinant Ser/Thr Fam20c Proteins in P. pastoris

The vectors were transformed into P. pastoris CBS7435, and putative strains were confirmed by colony PCR. The strains were grown overnight in BMGY (non-induction media), diluted to an OD600 of 1.0 in BMMY (induction media), and expression of recombinant Ser/Thr Fam20c proteins was monitored between 0 and 48 hours post-induction. At each 24-hour time point, cultures were supplemented with additional methanol to maintain induction conditions. Cultures were harvested, and the supernatant was retained for analysis.

### Example 4: Intracellular Expression of Bovine S1 Casein in PichiaPink™ Yeast Vector Design

For recombinant protein expression in yeast (P. pastoris), a recombinant vector encoding a bovine αS1 casein, variant B (SEQ ID NO: 4) was generated and codon-optimized for expression in P. pastoris. DNA encoding the αS1 casein of SEQ ID NO: 4 (which does not include N-terminal native secretion signal) was cloned into pPINKα-HC vector, which is an integrative vector targeting the ADE2 locus in P. pastoris. In one variant, the expression cassette included an N-terminal α-mating factor secretion signal, a FLAG tag for detection, and a C-terminal HiBiT tag, and expression was controlled by the pAOX1 promoter, which is an inducible promoter activated by the presence of methanol in the growth media. In a second variant, the expression cassette included an N-terminal Ost1 α-mating factor hybrid secretion signal, a FLAG tag, and a C-terminal HiBiT tag, and expression was controlled by the pAOX1 promoter. In a third variant, the expression cassette included an N-terminal α-mating factor secretion signal, a FLAG tag, and a C-terminal HiBiT tag, and expression was controlled by the pGAP promoter, which is a constitutive promoter. In a fourth variant, the expression cassette included an N-terminal Ost1 α-mating factor hybrid secretion signal, a FLAG tag, and a C-terminal HiBiT tag, and expression was controlled by the pGAP promoter.

### Intracellular Expression of αS1 Casein in PichiaPink™ Yeast

The vector above in which expression was controlled by the pGAP promoter was transformed into the PichiaPink™ (Strain 4) yeast strain, and putative recombinant strains were confirmed by colony PCR. The strains were grown overnight in YPD, diluted to an OD600 of 1.0 in YPD, and expression of αS1 casein proteins was monitored between 0 and 48 hours post-inoculation. At each 24-hour time point, cultures were supplemented with additional glucose to maintain growth conditions. Cultures were harvested. The cell pellets were lysed by bead beating. The soluble and insoluble intracellular protein fractions were analyzed by denaturing protein gel. Gels were transferred to a nitrocellulose membrane and probed with an anti-FLAG primary antibody conjugated to HRP.

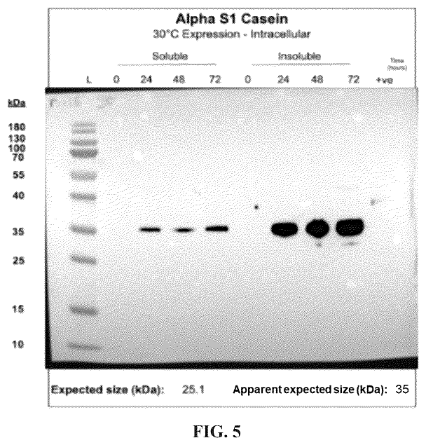

Shown in FIG. 5 is a western blot detecting intracellular expression of soluble and insoluble αS1 casein protein fractions under the control of the pGAP promoter extracted from transgenic PichiaPink™ yeast. The expression time points are indicated at the top of each lane. The expected weight for both the soluble and insoluble αS1 casein proteins is 25.1 kDA. FIG. 5 appears to show a full-length (intact) bovine αS1 casein protein expressed intracellularly in yeast cells.

### Example 5: Extracellular Expression of Human Osteopontin Protein in Escherichia coli Bacteria and Pichia pastoris Yeast

Vector Design and Osteopontin Protein Expression in E. coli

A recombinant vector encoding a human osteopontin (SEQ ID NO: 5) was generated. The expression cassette included a C-terminal 6×His tag (SEQ ID NO: 437) for detection and purification. The osteopontin protein expression was controlled by the T7 promoter, which is an inducible promoter activated by the presence of IPTG in the media. The vector was transformed into the BL21 Competent E. coli strain, and putative recombinant strains were confirmed by colony PCR.

Vector Design and Osteopontin Protein Expression in P. pastoris Yeast

For recombinant protein expression in yeast (Pichia pastoris (P. pastoris)), a recombinant vector encoding a human osteopontin (SEQ ID NO: 5) was generated and codon-optimized for expression in P. pastoris. DNA encoding the human osteopontin of SEQ ID NO: 5 (which does not include the N-terminal native secretion signal) was cloned into the pET24b vector for episomal expression in E. coli. The example constructs described were cloned into the yeast pD902 and pD915 vectors, which have an inducible pAOX1 promoter or a constitutive pGAP promoter, respectively. Both vectors contained the N-terminal α-mating factor secretion signal (SEQ ID NO: 312), a FLAG tag for detection and a C-terminal HiBiT tag, and expression was controlled by either the inducible pAOX1 promoter or the constitutive pGAP promoter.

The vectors above were transformed into P. pastoris CBS7435, and putative recombinant strains were confirmed by colony PCR. The strains were grown overnight in BMGY (non-induction media), diluted to an OD600 of 1.0 in BMMY (induction media), and expression of recombinant human osteopontin proteins were monitored between 0 and 48 hours post-induction. At each 24-hour time point, cultures were supplemented with additional methanol to maintain induction conditions. Cultures were harvested, and the supernatant was retained for analysis.

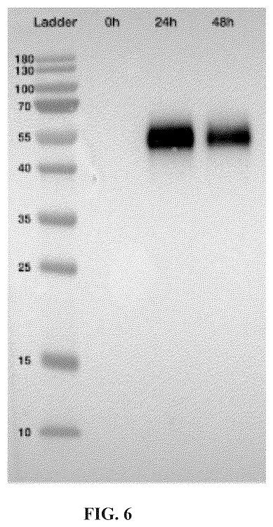

Shown in FIG. 6 is a Western Blot detecting extracellular expression of human osteopontin protein under the control of the pAOX1 promoter extracted from transgenic P. pastoris CBS7435 yeast. The expression time points are indicated at the top of each lane. FIG. 6 appears to show a full-length (intact) human osteopontin protein expressed extracellularly by yeast cells.

### Example 6: Engineered Human Serine/Threonine Protein Kinase Fam20c Variants for Fungal Expression

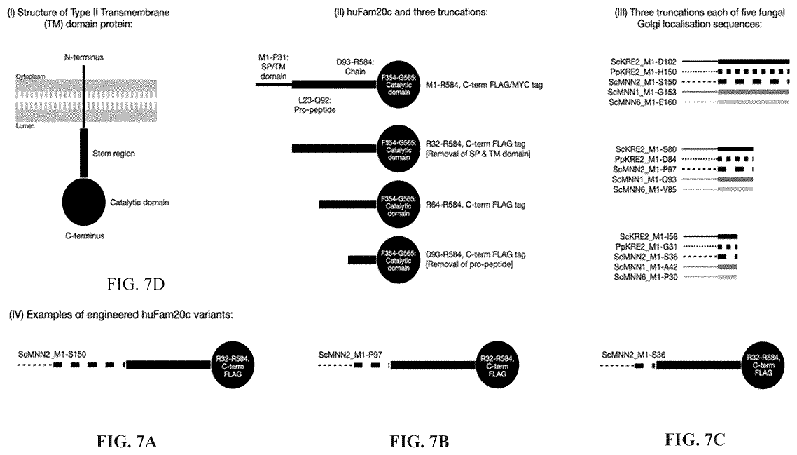

An approach for engineering human serine/threonine protein kinase Fam20c (Fam20c), a Type II transmembrane protein, for fungal expression is described herein (FIGS. 7A-7D). Type II transmembrane proteins can comprise a transmembrane domain, a catalytic domain, and a stem region, which is located between the transmembrane domain and the catalytic domain (FIG. 7A).

The approach leverages an ability to enhance protein activity by increasing the stability of huFam20c in fungal hosts. This can be achieved by fusing different lengths of the human Fam20c (huFam20c) stem region and catalytic domain (FIG. 7B) with different lengths of fungal transmembrane domains and stem regions (FIG. 7C), as membrane protein stability may be directly linked to the interaction between the side chains of amino acids and the phospholipid bilayer. This may yield fusion stem regions comprising fungal and human sequences (FIG. 7D). In some embodiments, the engineered huFam20c variants may comprise a C-terminal FLAG tag.

Different lengths of huFam20c comprising the stem region and catalytic domain can be generated by truncating huFam20c from the N-terminal end (FIG. 7B). The native signal peptide and the predicted transmembrane domain can be removed from the N-terminal end of huFam20c, thereby generating huFam20c truncant R32-R584. The native signal peptide, the predicted transmembrane domain, and a section of the propeptide region can be removed from the N-terminal end of huFam20c, thereby generating huFam20c truncant R64-R584. The native signal peptide, the predicted transmembrane domain, and the propeptide region can be removed from the N-terminal end of huFam20c, thereby generating huFam20c truncant D93-R584.

Different lengths of fungal transmembrane domains and stem regions can be generated by truncating various fungal Type II transmembrane proteins from the C-terminal end (FIG. 7C). Three lengths of Saccharomyces cerevisiae derived glycolipid 2-alpha-mannosyltransferase (ScKRE2) can be generated through truncations at the C-terminal to yield ScKRE2 comprising the first 102 amino acids (ScKRE_M1-D102), ScKRE2 comprising the first 80 amino acids (ScKRE_M1-S80), and ScKRE2 comprising the first 58 amino acids (ScKRE_M1-158). Three lengths of Pichia pastoris derived glycolipid 2-alpha-mannosyltransferase (PpKRE2) can be generated through truncations at the C-terminal to yield PpKRE2 comprising the first 150 amino acids (PpKRE_M1-H150), PpKRE2 comprising the first 84 amino acids (PpKRE_M1-D84), and PpKRE2 comprising the first 31 amino acids (PpKRE_M1-G31). Three lengths of Saccharomyces cerevisiae derived alpha 1,2-mannosyltransferase (ScMNN2) can be generated through truncations at the C-terminal to yield ScMNN2 comprising the first 150 amino acids (ScMNN2_M1-S150), ScMNN2 comprising the first 97 amino acids (ScMNN2_M1-P97), and ScMNN2 comprising the first 36 amino acids (ScMNN2_M1-S36). Three lengths of Saccharomyces cerevisiae derived alpha alpha1,3-mannosyltransferase (ScMNN1) can be generated through truncations at the C-terminal to yield ScMNN1 comprising the first 153 amino acids (ScMNN1_M1-G153), ScMNN1 comprising the first 93 amino acids (ScMNN1_M1-Q93), and ScMNN1 comprising the first 42 amino acids (ScMNN1_M1-A42). Three lengths of Saccharomyces cerevisiae derived mannosyltransferase (ScMNN6) can be generated through truncations at the C-terminal to yield ScMNN6 comprising the first 160 amino acids (ScMNN6_M1-E160), ScMNN6 comprising the first 85 amino acids (ScMNN6_M1-V85), and ScMNN6 comprising the first 30 amino acids (ScMNN6_M1-P30).

Membrane anchored huFam20c variants were engineered through combinatorial fusions of different lengths of huFam20c comprising the stem region and catalytic domain with different lengths of fungal transmembrane domains and stem regions (FIG. 7D). Membrane anchored huFam20c variants that were generated are shown in Table 10. The polynucleic acid sequences encoding the engineered kinases are shown in Table 11.

TABLE 10 provides engineered kinases.

TABLE 10
Engineered kinases

| SEQ | Membrane | | | |
|---|---|---|---|---|
| ID | anchoring | | Construct | |
| NO: | domain | Kinase | Name | Amino acid sequence |
| 93 | Native | Fam20c | Fam20c(M | MKMMLVRRFRVLILMVFLVACALHIAL |
| | Fam20c | (M1- | 1- | DLLPRLERRGARPSGEPGCSCAQPAAEV |
| | derived | R584) | R584)_FLAG | AAPGWAQVRGRPGEPPAASSAAGDAGW |
| | | | | PNKHTLRILQDFSSDPSSNLSSHSLEKLPP |
| | | | | AAEPAERALRGRDPGALRPHDPAHRPLL |
| | | | | RDPGPRRSESPPGPGGDASLLARLFEHPL |
| | | | | YRVAVPPLTEEDVLFNVNSDTRLSPKAAE |
| | | | | NPDWPHAGAEGAEFLSPGEAAVDSYPNW |
| | | | | LKFHIGINRYELYSRHNPAIEALLHDLSSQ |
| | | | | RITSVAMKSGGTQLKLIMTFQNYGQALF |
| | | | | KPMKQTREQETPPDFFYFSDYERHNAEI |
| | | | | AAFHLDRILDFRRVPPVAGRMVNMTKEI |
| | | | | RDVTRDKKLWRTFFISPANNICFYGECSY |
| | | | | YCSTEHALCGKPDQIEGSLAAFLPDLSLA |
| | | | | KRKTWRNPWRRSYHKRKKAEWEVDPD |
| | | | | YCEEVKQTPPYDSSHRILDVMDMTIFDFL |
| | | | | MGNMDRHHYETFEKFGNETFIIHLDNGR |
| | | | | GFGKYSHDELSILVPLQQCCRIRKSTYLR |
| | | | | LQLLAKEEYKLSLLMAESLRGDQVAPVL |
| | | | | YQPHLEALDRRLRVVLKAVRDCVERNG |
| | | | | LHSVVDDDLDTEHRAASARDYKDDDDK |
| 94 | N/A | Fam20c | Fam20c(R | MRLERRGARPSGEPGCSCAQPAAEVAAP |
| | | (R32- | 32- | GWAQVRGRPGEPPAASSAAGDAGWPNK |
| | | R584) | R584)_FLAG | HTLRILQDFSSDPSSNLSSHSLEKLPPAAE |
| | | | | PAERALRGRDPGALRPHDPAHRPLLRDP |
| | | | | GPRRSESPPGPGGDASLLARLFEHPLYRV |
| | | | | AVPPLTEEDVLFNVNSDTRLSPKAAENPD |
| | | | | WPHAGAEGAEFLSPGEAAVDSYPNWLKF |
| | | | | HIGINRYELYSRHNPAIEALLHDLSSQRIT |
| | | | | SVAMKSGGTQLKLIMTFQNYGQALFKP |
| | | | | MKQTREQETPPDFFYFSDYERHNAEIAAF |
| | | | | HLDRILDFRRVPPVAGRMVNMTKEIRDV |
| | | | | TRDKKLWRTFFISPANNICFYGECSYYCS |
| | | | | TEHALCGKPDQIEGSLAAFLPDLSLAKRK |
| | | | | TWRNPWRRSYHKRKKAEWEVDPDYCEE |
| | | | | VKQTPPYDSSHRILDVMDMTIFDFLMGN |
| | | | | MDRHHYETFEKFGNETFIIHLDNGRGFG |
| | | | | KYSHDELSILVPLQQCCRIRKSTYLRLQL |
| | | | | LAKEEYKLSLLMAESLRGDQVAPVLYQP |
| | | | | HLEALDRRLRVVLKAVRDCVERNGLHSV |
| | | | | VDDDLDTEHRAASARDYKDDDDK |
| 95 | ScKRE2 | Fam20c | ScKRE(M | MALFLSKRLLRFTVIAGAVIVLLLTLNSN |
| | (M1-158) | (R32- | 1-158)_ | SRTQQYIPSSISAAFDFTSGSISPEQQVIRL |
| | | R584) | Fam20c | ERRGARPSGEPGCSCAQPAAEVAAPGWA |
| | | | (R32- | QVRGRPGEPPAASSAAGDAGWPNKHTL |
| | | | R584)_FLAG | RILQDFSSDPSSNLSSHSLEKLPPAAEPAE |
| | | | | RALRGRDPGALRPHDPAHRPLLRDPGPR |
| | | | | RSESPPGPGGDASLLARLFEHPLYRVAVP |
| | | | | PLTEEDVLFNVNSDTRLSPKAAENPDWP |
| | | | | HAGAEGAEFLSPGEAAVDSYPNWLKFHI |
| | | | | GINRYELYSRANPAIEALLHDLSSQRITSV |
| | | | | AMKSGGTQLKLIMTFQNYGQALFKPMK |
| | | | | QTREQETPPDFFYFSDYERHNAEIAAFHL |
| | | | | DRILDFRRVPPVAGRMVNMTKEIRDVTR |
| | | | | DKKLWRTFFISPANNICFYGECSYYCSTE |
| | | | | HALCGKPDQIEGSLAAFLPDLSLAKRKT |
| | | | | WRNPWRRSYHKRKKAEWEVDPDYCEE |
| | | | | VKQTPPYDSSHRILDVMDMTIFDFLMGN |
| | | | | MDRHHYETFEKFGNETFIIHLDNGRGFG |
| | | | | KYSHDELSILVPLQQCCRIRKSTYLRLQL |
| | | | | LAKEEYKLSLLMAESLRGDQVAPVLYQP |
| | | | | HLEALDRRLRVVLKAVRDCVERNGLHSV |
| | | | | VDDDLDTEHRAASARDYKDDDDK |
| 96 | ScKRE2 | Fam20c | ScKRE2( | MALFLSKRLLRFTVIAGAVIVLLLTLNSN |
| | (M1-S80) | (R32- | M1-S80)_ | SRTQQYIPSSISAAFDFTSGSISPEQQVISE |
| | | R584) | Fam20c | ENDAKKLEQSALNSEASEDSRLERRGAR |
| | | | (R32- | PSGEPGCSCAQPAAEVAAPGWAQVRGRP |
| | | | R584)_FLAG | GEPPAASSAAGDAGWPNKHTLRILQDESS |
| | | | | DPSSNLSSHSLEKLPPAAEPAERALRGRD |
| | | | | PGALRPHDPAHRPLLRDPGPRRSESPPGP |
| | | | | GGDASLLARLFEHPLYRVAVPPLTEEDV |
| | | | | LFNVNSDTRLSPKAAENPDWPHAGAEGA |
| | | | | EFLSPGEAAVDSYPNWLKFHIGINRYELY |
| | | | | SRHNPAIEALLHDLSSQRITSVAMKSGGT |
| | | | | QLKLIMTFQNYGQALFKPMKQTREQET |
| | | | | PPDFFYFSDYERHNAEIAAFHLDRILDER |
| | | | | RVPPVAGRMVNMTKEIRDVTRDKKLWR |
| | | | | TFFISPANNICFYGECSYYCSTEHALCGKP |
| | | | | DQIEGSLAAFLPDLSLAKRKTWRNPWRR |
| | | | | SYHKRKKAEWEVDPDYCEEVKQTPPYDS |
| | | | | SHRILDVMDMTIFDFLMGNMDRHHYETF |
| | | | | EKFGNETFIIHLDNGRGFGKYSHDELSIL |
| | | | | VPLQQCCRIRKSTYLRLQLLAKEEYKLS |
| | | | | LLMAESLRGDQVAPVLYQPHLEALDRRL |
| | | | | RVVLKAVRDCVERNGLHSVVDDDLDTE |
| | | | | HRAASARDYKDDDDK |
| 97 | ScKRE2 | Fam20c | ScKRE2( | MALFLSKRLLRFTVIAGAVIVLLLTLNSN |
| | (M1-D102) | (R32- | M1- | SRTQQYIPSSISAAFDFTSGSISPEQQVISE |
| | | R584) | D102)_Fa | ENDAKKLEQSALNSEASEDSEAMDEESK |
| | | | m20c(R32- | ALKAAAEKADAPIDRLERRGARPSGEPG |
| | | | R584)_FLAG | CSCAQPAAEVAAPGWAQVRGRPGEPPA |
| | | | | ASSAAGDAGWPNKHTLRILQDFSSDPSSN |
| | | | | LSSHSLEKLPPAAEPAERALRGRDPGALR |
| | | | | PHDPAHRPLLRDPGPRRSESPPGPGGDAS |
| | | | | LLARLFEHPLYRVAVPPLTEEDVLFNVNS |
| | | | | DTRLSPKAAENPDWPHAGAEGAEFLSPG |
| | | | | EAAVDSYPNWLKFHIGINRYELYSRHINPA |
| | | | | IEALLHDLSSQRITSVAMKSGGTQLKLIM |
| | | | | TFQNYGQALFKPMKQTREQETPPDFFYF |
| | | | | SDYERHNAEIAAFHLDRILDFRRVPPVAG |
| | | | | RMVNMTKEIRDVTRDKKLWRTFFISPAN |
| | | | | NICFYGECSYYCSTEHALCGKPDQIEGSL |
| | | | | AAFLPDLSLAKRKTWRNPWRRSYHKRK |
| | | | | KAEWEVDPDYCEEVKQTPPYDSSHRILD |
| | | | | VMDMTIFDFLMGNMDRHHYETFEKFGN |
| | | | | ETFIIHLDNGRGFGKYSHDELSILVPLQQ |
| | | | | CCRIRKSTYLRLQLLAKEEYKLSLLMAE |
| | | | | SLRGDQVAPVLYQPHLEALDRRLRVVLK |
| | | | | AVRDCVERNGLHSVVDDDLDTEHRAASA |
| | | | | RDYKDDDDK |
| 98 | PpKRE2 | Fam20c | PpKRE2( | MVHIGFRSLKAVFILALSSLILYGIVTTFD |
| | (M1-G31) | (R32- | M1- | GRLERRGARPSGEPGCSCAQPAAEVAAP |
| | | R584) | G31)_Fam | GWAQVRGRPGEPPAASSAAGDAGWPNK |
| | | | 20c(R32- | HTLRILQDFSSDPSSNLSSHSLEKLPPAAE |
| | | | R584)_FLAG | PAERALRGRDPGALRPHDPAHRPLLRDP |
| | | | | GPRRSESPPGPGGDASLLARLFEHPLYRV |
| | | | | AVPPLTEEDVLFNVNSDTRLSPKAAENPD |
| | | | | WPHAGAEGAEFLSPGEAAVDSYPNWLKF |
| | | | | HIGINRYELYSRHNPAIEALLHDLSSQRIT |
| | | | | SVAMKSGGTQLKLIMTFQNYGQALFKP |
| | | | | MKQTREQETPPDFFYFSDYERHNAEIAAF |
| | | | | HLDRILDFRRVPPVAGRMVNMTKEIRDV |
| | | | | TRDKKLWRTFFISPANNICFYGECSYYCS |
| | | | | TEHALCGKPDQIEGSLAAFLPDLSLAKRK |
| | | | | TWRNPWRRSYHKRKKAEWEVDPDYCEE |
| | | | | VKQTPPYDSSHRILDVMDMTIFDFLMGN |
| | | | | MDRHHYETFEKFGNETFIIHLDNGRGFG |
| | | | | KYSHDELSILVPLQQCCRIRKSTYLRLQL |
| | | | | LAKEEYKLSLLMAESLRGDQVAPVLYQP |
| | | | | HLEALDRRLRVVLKAVRDCVERNGLHSV |
| | | | | VDDDLDTEHRAASARDYKDDDDK |
| 99 | PpKRE2 | Fam20c | PpKRE2( | MVHIGFRSLKAVFILALSSLILYGIVTTFD |
| | (M1-D84) | (R32- | M1- | GSRASRYQPPYVNHSQDPLYHSGNSYNR |
| | | R584) | D84)_Fam | ENATFVTLCRNEDLYSHIQSIKKVEDRLER |
| | | | 20c(R32- | RGARPSGEPGCSCAQPAAEVAAPGWAQ |
| | | | R584)_FLAG | VRGRPGEPPAASSAAGDAGWPNKHTLRI |
| | | | | LQDFSSDPSSNLSSHSLEKLPPAAEPAERA |
| | | | | LRGRDPGALRPHDPAHRPLLRDPGPRRS |
| | | | | ESPPGPGGDASLLARLFEHPLYRVAVPPL |
| | | | | TEEDVLFNVNSDTRLSPKAAENPDWPHA |
| | | | | GAEGAEFLSPGEAAVDSYPNWLKFHIGIN |
| | | | | RYELYSRHNPAIEALLHDLSSQRITSVAM |
| | | | | KSGGTQLKLIMTFQNYGQALFKPMKQT |
| | | | | REQETPPDFFYFSDYERHNAEIAAFHLDR |
| | | | | ILDFRRVPPVAGRMVNMTKEIRDVTRDK |
| | | | | KLWRTFFISPANNICFYGECSYYCSTEHA |
| | | | | LCGKPDQIEGSLAAFLPDLSLAKRKTWR |
| | | | | NPWRRSYHKRKKAEWEVDPDYCEEVKQ |
| | | | | TPPYDSSHRILDVMDMTIFDFLMGNMDR |
| | | | | HHYETFEKFGNETFIHLDNGRGFGKYSH |
| | | | | DELSILVPLQQCCRIRKSTYLRLQLLAKE |
| | | | | EYKLSLLMAESLRGDQVAPVLYQPHLEA |
| | | | | LDRRLRVVLKAVRDCVERNGLHSVVDD |
| | | | | DLDTEHRAASARDYKDDDDK |
| 100 | PpKRE2 | Fam20c | PpKRE2( | MVHIGFRSLKAVFILALSSLILYGIVTTED |
| | (M1-H150) | (R32- | M1- | GSRASRYQPPYVNHSQDPLYHSGNSYNR |
| | | R584) | H150)_Fa | ENATFVTLCRNEDLYSIIQSIKKVEDRENN |
| | | | m20c(R32- | KFAYDWVFLNEVPFTDEFKERTSVLISGQ |
| | | | R584)_FLAG | AKYGLIPKEHWSYPDYIDQERAAESRRQ |
| | | | | LEDQHRLERRGARPSGEPGCSCAQPAAE |
| | | | | VAAPGWAQVRGRPGEPPAASSAAGDAG |
| | | | | WPNKHTLRILQDFSSDPSSNLSSHSLEKLP |
| | | | | PAAEPAERALRGRDPGALRPHDPAHRPL |
| | | | | LRDPGPRRSESPPGPGGDASLLARLFEHP |
| | | | | LYRVAVPPLTEEDVLFNVNSDTRLSPKAA |
| | | | | ENPDWPHAGAEGAEFLSPGEAAVDSYPN |
| | | | | WLKFHIGINRYELYSRANPAIEALLHDLS |
| | | | | SQRITSVAMKSGGTQLKLIMTFQNYGQA |
| | | | | LFKPMKQTREQETPPDFFYFSDYERHNA |
| | | | | EIAAFHLDRILDFRRVPPVAGRMVNMTK |
| | | | | EIRDVTRDKKLWRTFFISPANNICFYGEC |
| | | | | SYYCSTEHALCGKPDQIEGSLAAFLPDLS |
| | | | | LAKRKTWRNPWRRSYHKRKKAEWEVD |
| | | | | PDYCEEVKQTPPYDSSHRILDVMDMTIFD |
| | | | | FLMGNMDRHHYETFEKFGNETFIIHLDN |
| | | | | GRGFGKYSHDELSILVPLQQCCRIRKSTY |
| | | | | LRLQLLAKEEYKLSLLMAESLRGDQVAP |
| | | | | VLYQPHLEALDRRLRVVLKAVRDCVERN |
| | | | | GLHSVVDDDLDTEHRAASARDYKDDDD |
| | | | | K |
| 101 | ScMNN2 | Fam20c | ScMNN2( | MLLTKRFSKLFKLTFIVLILCGLFVITNK |
| | (M1-S36) | (R32- | M1- | YMDENTSRLERRGARPSGEPGCSCAQPA |
| | | R584) | S36)_Fam | AEVAAPGWAQVRGRPGEPPAASSAAGDA |
| | | | 20c(R32- | GWPNKHTLRILQDFSSDPSSNLSSHSLEK |
| | | | R584)_FLAG | LPPAAEPAERALRGRDPGALRPHDPAHR |
| | | | | PLLRDPGPRRSESPPGPGGDASLLARLFE |
| | | | | HPLYRVAVPPLTEEDVLFNVNSDTRLSPK |
| | | | | AAENPDWPHAGAEGAEFLSPGEAAVDSY |
| | | | | PNWLKFHIGINRYELYSRHNPAIEALLHD |
| | | | | LSSQRITSVAMKSGGTQLKLIMTFQNYG |
| | | | | QALFKPMKQTREQETPPDFFYFSDYERH |
| | | | | NAEIAAFHLDRILDFRRVPPVAGRMVNM |
| | | | | TKEIRDVTRDKKLWRTFFISPANNICFYG |
| | | | | ECSYYCSTEHALCGKPDQIEGSLAAFLPD |
| | | | | LSLAKRKTWRNPWRRSYHKRKKAEWE |
| | | | | VDPDYCEEVKQTPPYDSSHRILDVMDMT |
| | | | | IFDFLMGNMDRHHYETFEKFGNETFIIHL |
| | | | | DNGRGFGKYSHDELSILVPLQQCCRIRKS |
| | | | | TYLRLQLLAKEEYKLSLLMAESLRGDQV |
| | | | | APVLYQPHLEALDRRLRVVLKAVRDCVE |
| | | | | RNGLHSVVDDDLDTEHRAASARDYKDDD |
| | | | | DK |
| 102 | ScMINN2 | Fam20c | ScMNN2( | MLLTKRFSKLFKLTFIVLILCGLFVITNK |
| | (M1-P97) | (R32- | M1- | YMDENTSVKEYKEYLDRYVQSYSNKYSS |
| | | R584) | P97)_Fam | SSDAASADDSTPLRDNDEAGNEKLKSFYN |
| | | | 20c(R32- | NVENFLMVDSPRLERRGARPSGEPGCSC |
| | | | R584)_FLAG | AQPAAEVAAPGWAQVRGRPGEPPAASSA |
| | | | | AGDAGWPNKHTLRILQDFSSDPSSNLSSH |
| | | | | SLEKLPPAAEPAERALRGRDPGALRPHD |
| | | | | PAHRPLLRDPGPRRSESPPGPGGDASLLA |
| | | | | RLFEHPLYRVAVPPLTEEDVLFNVNSDTR |
| | | | | LSPKAAENPDWPHAGAEGAEFLSPGEAA |
| | | | | VDSYPNWLKFHIGINRYELYSRHNPAIEA |
| | | | | LLHDLSSQRITSVAMKSGGTQLKLIMTF |
| | | | | QNYGQALFKPMKQTREQETPPDFFYFSD |
| | | | | YERHNAEIAAFHLDRILDFRRVPPVAGR |
| | | | | MVNMTKEIRDVTRDKKLWRTFFISPANN |
| | | | | ICFYGECSYYCSTEHALCGKPDQIEGSLA |
| | | | | AFLPDLSLAKRKTWRNPWRRSYHKRKK |
| | | | | AEWEVDPDYCEEVKQTPPYDSSHRILDV |
| | | | | MDMTIFDFLMGNMDRHHYETFEKEGNE |
| | | | | TFIIHLDNGRGFGKYSHDELSILVPLQQC |
| | | | | CRIRKSTYLRLQLLAKEEYKLSLLMAESL |
| | | | | RGDQVAPVLYQPHLEALDRRLRVVLKA |
| | | | | VRDCVERNGLHSVVDDDLDTEHRAASAR |
| | | | | DYKDDDDK |
| 103 | ScMNN2 | Fam20c | ScMNN2( | MLLTKRFSKLFKLTFIVLILCGLFVITNK |
| | (M1-$150) | (R32- | M1- | YMDENTSVKEYKEYLDRYVQSYSNKYSS |
| | | R584) | S150)_Fa | SSDAASADDSTPLRDNDEAGNEKLKSFYN |
| | | | m20c(R32- | NVFNFLMVDSPKGSTAKQYNEACLLKGD |
| | | | R584)_FLAG | IGDRPDHYKDLYKLSAKELSKCLELSPDE |
| | | | | VASLTKSRLERRGARPSGEPGCSCAQPA |
| | | | | AEVAAPGWAQVRGRPGEPPAASSAAGDA |
| | | | | GWPNKHTLRILQDFSSDPSSNLSSHSLEK |
| | | | | LPPAAEPAERALRGRDPGALRPHDPAHR |
| | | | | PLLRDPGPRRSESPPGPGGDASLLARLFE |
| | | | | HPLYRVAVPPLTEEDVLFNVNSDTRLSPK |
| | | | | AAENPDWPHAGAEGAEFLSPGEAAVDSY |
| | | | | PNWLKFHIGINRYELYSRANPAIEALLHD |
| | | | | LSSQRITSVAMKSGGTQLKLIMTFQNYG |
| | | | | QALFKPMKQTREQETPPDFFYFSDYERH |
| | | | | NAEIAAFHLDRILDFRRVPPVAGRMVNM |
| | | | | TKEIRDVTRDKKLWRTFFISPANNICFYG |
| | | | | ECSYYCSTEHALCGKPDQIEGSLAAFLPD |
| | | | | LSLAKRKTWRNPWRRSYHKRKKAEWE |
| | | | | VDPDYCEEVKQTPPYDSSHRILDVMDMT |
| | | | | IFDFLMGNMDRHHYETFEKFGNETFUHL |
| | | | | DNGRGFGKYSHDELSILVPLQQCCRIRKS |
| | | | | TYLRLQLLAKEEYKLSLLMAESLRGDQV |
| | | | | APVLYQPHLEALDRRLRVVLKAVRDCVE |
| | | | | RNGLHSVVDDDLDTEHRAASARDYKDDD |
| | | | | DK |
| 104 | ScMNN1 | Fam20c | ScMNN1( | MLALRRFILNQRSLRSCTIPILVGALIILV |
| | (M1-A42) | (R32- | M1- | LFQLVTHRNDARLERRGARPSGEPGCSC |
| | | R584) | A42)_Fam | AQPAAEVAAPGWAQVRGRPGEPPAASSA |
| | | | 20c(R32- | AGDAGWPNKHTLRILQDFSSDPSSNLSSH |
| | | | R584)_FLAG | SLEKLPPAAEPAERALRGRDPGALRPHD |
| | | | | PAHRPLLRDPGPRRSESPPGPGGDASLLA |
| | | | | RLFEHPLYRVAVPPLTEEDVLFNVNSDTR |
| | | | | LSPKAAENPDWPHAGAEGAEFLSPGEAA |
| | | | | VDSYPNWLKFHIGINRYELYSRHNPAIEA |
| | | | | LLHDLSSQRITSVAMKSGGTQLKLIMTF |
| | | | | QNYGQALFKPMKQTREQETPPDFFYFSD |
| | | | | YERHNAEIAAFHLDRILDFRRVPPVAGR |
| | | | | MVNMTKEIRDVTRDKKLWRTFFISPANN |
| | | | | ICFYGECSYYCSTEHALCGKPDQIEGSLA |
| | | | | AFLPDLSLAKRKTWRNPWRRSYHKRKK |
| | | | | AEWEVDPDYCEEVKQTPPYDSSHRILDV |
| | | | | MDMTIFDFLMGNMDRHHYETFEKFGNE |
| | | | | TFIIHLDNGRGFGKYSHDELSILVPLQQC |
| | | | | CRIRKSTYLRLQLLAKEEYKLSLLMAESL |
| | | | | RGDQVAPVLYQPHLEALDRRLRVVLKA |
| | | | | VRDCVERNGLHSVVDDDLDTEHRAASAR |
| | | | | DYKDDDDK |
| 105 | ScMNN1 | Fam20c | ScMNN1 | MLALRRFILNQRSLRSCTIPILVGALIILV |
| | (M1-Q93) | (R32- | (M1-Q93)_ | LFQLVTHRNDALIRSSNVNSTNKKTLKDA |
| | | R584) | Fam20c | DPKVLIEAFGSPEVDPVDTIPVSPLELVPF |
| | | | (R32- | YDQRLERRGARPSGEPGCSCAQPAAEVA |
| | | | R584)_FLAG | APGWAQVRGRPGEPPAASSAAGDAGWP |
| | | | | NKHTLRILQDFSSDPSSNLSSHSLEKLPPA |
| | | | | AEPAERALRGRDPGALRPHDPAHRPLLR |
| | | | | DPGPRRSESPPGPGGDASLLARLFEHPLY |
| | | | | RVAVPPLTEEDVLFNVNSDTRLSPKAAEN |
| | | | | PDWPHAGAEGAEFLSPGEAAVDSYPNWL |
| | | | | KFHIGINRYELYSRUNPAIEALLHDLSSQ |
| | | | | RITSVAMKSGGTQLKLIMTFQNYGQALF |
| | | | | KPMKQTREQETPPDFFYFSDYERHNAEI |
| | | | | AAFHLDRILDFRRVPPVAGRMVNMTKEI |
| | | | | RDVTRDKKLWRTFFISPANNICFYGECSY |
| | | | | YCSTEHALCGKPDQIEGSLAAFLPDLSLA |
| | | | | KRKTWRNPWRRSYHKRKKAEWEVDPD |
| | | | | YCEEVKQTPPYDSSHRILDVMDMTIFDFL |
| | | | | MGNMDRHHYETFEKFGNETFIIHLDNGR |
| | | | | GFGKYSHDELSILVPLQQCCRIRKSTYLR |
| | | | | LQLLAKEEYKLSLLMAESLRGDQVAPVL |
| | | | | YQPHLEALDRRLRVVLKAVRDCVERNG |
| | | | | LHSVVDDDLDTEHRAASARDYKDDDDK |
| 106 | ScMNN1 | Fam20c | ScMINNI( | MLALRRFILNQRSLRSCTIPILVGALIIILV |
| | (M1-G153) | (R32- | M1- | LFQLVTHRNDALIRSSNVNSTNKKTLKDA |
| | | R584) | G153)_Fa | DPKVLIEAFGSPEVDPVDTIPVSPLELVPF |
| | | | m20c(R32- | YDQSIDTKRSSSWLINKKGYYKHFNELSL |
| | | | R584)_FLAG | TDRCKFYFRTLYTLDDEWTNSVKKLEYS |
| | | | | INDNEGRLERRGARPSGEPGCSCAQPAA |
| | | | | EVAAPGWAQVRGRPGEPPAASSAAGDA |
| | | | | GWPNKHTLRILQDFSSDPSSNLSSHSLEK |
| | | | | LPPAAEPAERALRGRDPGALRPHDPAHR |
| | | | | PLLRDPGPRRSESPPGPGGDASLLARLFE |
| | | | | HPLYRVAVPPLTEEDVLFNVNSDTRLSPK |
| | | | | AAENPDWPHAGAEGAEFLSPGEAAVDSY |
| | | | | PNWLKFHIGINRYELYSRANPAIEALLHD |
| | | | | LSSQRITSVAMKSGGTQLKLIMTFQNYG |
| | | | | QALFKPMKQTREQETPPDFFYFSDYERH |
| | | | | NAEIAAFHLDRILDFRRVPPVAGRMVNM |
| | | | | TKEIRDVTRDKKLWRTFFISPANNICFYG |
| | | | | ECSYYCSTEHALCGKPDQIEGSLAAFLPD |
| | | | | LSLAKRKTWRNPWRRSYHKRKKAEWE |
| | | | | VDPDYCEEVKQTPPYDSSHRILDVMDMT |
| | | | | IFDFLMGNMDRHHYETFEKFGNETFIIHL |
| | | | | DNGRGFGKYSHDELSILVPLQQCCRIRKS |
| | | | | TYLRLQLLAKEEYKLSLLMAESLRGDQV |
| | | | | APVLYQPHLEALDRRLRVVLKAVRDCVE |
| | | | | RNGLHSVVDDDLDTEHRAASARDYKDDD |
| | | | | DK |
| 107 | ScMNN6 | Fam20c | ScMINN6( | MHVLLSKKIARFLLISFVFVLALMVTINH |
| | (M1-P30) | (R32- | M1- | PRLERRGARPSGEPGCSCAQPAAEVAAP |
| | | R584) | P30)_Fam | GWAQVRGRPGEPPAASSAAGDAGWPNK |
| | | | 20c(R32- | HTLRILQDFSSDPSSNLSSHSLEKLPPAAE |
| | | | R584)_FLAG | PAERALRGRDPGALRPHDPAHRPLLRDP |
| | | | | GPRRSESPPGPGGDASLLARLFEHPLYRV |
| | | | | AVPPLTEEDVLFNVNSDTRLSPKAAENPD |
| | | | | WPHAGAEGAEFLSPGEAAVDSYPNWLKF |
| | | | | HIGINRYELYSRHNPAIEALLHDLSSQRIT |
| | | | | SVAMKSGGTQLKLIMTFQNYGQALFKP |
| | | | | MKQTREQETPPDFFYFSDYERHNAEIAAF |
| | | | | HLDRILDFRRVPPVAGRMVNMTKEIRDV |
| | | | | TRDKKLWRTFFISPANNICFYGECSYYCS |
| | | | | TEHALCGKPDQIEGSLAAFLPDLSLAKRK |
| | | | | TWRNPWRRSYHKRKKAEWEVDPDYCEE |
| | | | | VKQTPPYDSSHRILDVMDMTIFDFLMGN |
| | | | | MDRHHYETFEKFGNETFIIHLDNGRGFG |
| | | | | KYSHDELSILVPLQQCCRIRKSTYLRLQL |
| | | | | LAKEEYKLSLLMAESLRGDQVAPVLYQP |
| | | | | HLEALDRRLRVVLKAVRDCVERNGLHSV |
| | | | | VDDDLDTEHRAASARDYKDDDDK |
| 108 | ScMINN6 | Fam20c | ScMNN6( | MHVLLSKKIARFLLISFVFVLALMVTINH |
| | (M1-V85) | (R32- | M1- | PKTKQMSEQYVTPYLPKSLQPIAKISAEE |
| | | R584) | V85)_Fam | QRRIQSEQEEAELKQSLEGEAIRNATVRL |
| | | | 20c(R32- | ERRGARPSGEPGCSCAQPAAEVAAPGWA |
| | | | R584)_FLAG | QVRGRPGEPPAASSAAGDAGWPNKHTL |
| | | | | RILQDFSSDPSSNLSSHSLEKLPPAAEPAE |
| | | | | RALRGRDPGALRPHDPAHRPLLRDPGPR |
| | | | | RSESPPGPGGDASLLARLFEHPLYRVAVP |
| | | | | PLTEEDVLFNVNSDTRLSPKAAENPDWP |
| | | | | HAGAEGAEFLSPGEAAVDSYPNWLKFHI |
| | | | | GINRYELYSRHNPAIEALLHDLSSQRITSV |
| | | | | AMKSGGTQLKLIMTFQNYGQALFKPMK |
| | | | | QTREQETPPDFFYFSDYERHNAEIAAFHL |
| | | | | DRILDFRRVPPVAGRMVNMTKEIRDVTR |
| | | | | DKKLWRTFFISPANNICFYGECSYYCSTE |
| | | | | HALCGKPDQIEGSLAAFLPDLSLAKRKT |
| | | | | WRNPWRRSYHKRKKAEWEVDPDYCEE |
| | | | | VKQTPPYDSSHRILDVMDMTIFDFLMGN |
| | | | | MDRHHYETFEKFGNETFIIHLDNGRGFG |
| | | | | KYSHDELSILVPLQQCCRIRKSTYLRLQL |
| | | | | LAKEEYKLSLLMAESLRGDQVAPVLYQP |
| | | | | HLEALDRRLRVVLKAVRDCVERNGLASV |
| | | | | VDDDLDTEHRAASARDYKDDDDK |
| 109 | ScMINN6 | Fam20c | ScMNN6( | MHVLLSKKIARFLLISFVFVLALMVTINH |
| | (M1-E160) | (R32- | M1- | PKTKQMSEQYVTPYLPKSLQPIAKISAEE |
| | | R584) | E160)_Fa | QRRIQSEQEEAELKQSLEGEAIRNATVNA |
| | | | m20c(R32- | IKEKIKSYGGNETTLGFMVPSYINHRGSP |
| | | | R584)_FLAG | PKACFVSLITERDSMTQILQSIDEVQVKF |
| | | | | NKNFAYPWVFISQGERLERRGARPSGEP |
| | | | | GCSCAQPAAEVAAPGWAQVRGRPGEPP |
| | | | | AASSAAGDAGWPNKHTLRILQDFSSDPSS |
| | | | | NLSSHSLEKLPPAAEPAERALRGRDPGAL |
| | | | | RPHDPAHRPLLRDPGPRRSESPPGPGGDA |
| | | | | SLLARLFEHPLYRVAVPPLTEEDVLENVN |
| | | | | SDTRLSPKAAENPDWPHAGAEGAEFLSP |
| | | | | GEAAVDSYPNWLKFHIGINRYELYSRHN |
| | | | | PAIEALLHDLSSQRITSVAMKSGGTQLKL |
| | | | | IMTFQNYGQALFKPMKQTREQETPPDFF |
| | | | | YFSDYERHNAEIAAFHLDRILDFRRVPPV |
| | | | | AGRMVNMTKEIRDVTRDKKLWRTFFISP |
| | | | | ANNICFYGECSYYCSTEHALCGKPDQIEG |
| | | | | SLAAFLPDLSLAKRKTWRNPWRRSYHKR |
| | | | | KKAEWEVDPDYCEEVKQTPPYDSSHRIL |
| | | | | DVMDMTIFDFLMGNMDRHHYETFEKFG |
| | | | | NETFIIHLDNGRGFGKYSHDELSILVPLQ |
| | | | | QCCRIRKSTYLRLQLLAKEEYKLSLLMA |
| | | | | ESLRGDQVAPVLYQPHLEALDRRLRVVL |
| | | | | KAVRDCVERNGLHSVVDDDLDTEHRAAS |
| | | | | ARDYKDDDDK |
| 110 | N/A | Fam20c | Fam20c(R | MRGRPGEPPAASSAAGDAGWPNKHTLRI |
| | | (R64- | 64- | LQDFSSDPSSNLSSHSLEKLPPAAEPAERA |
| | | R584) | R584)_FLAG | LRGRDPGALRPHDPAHRPLLRDPGPRRS |
| | | | | ESPPGPGGDASLLARLFEHPLYRVAVPPL |
| | | | | TEEDVLFNVNSDTRLSPKAAENPDWPHA |
| | | | | GAEGAEFLSPGEAAVDSYPNWLKFHIGIN |
| | | | | RYELYSRHNPAIEALLHDLSSQRITSVAM |
| | | | | KSGGTQLKLIMTFQNYGQALFKPMKQT |
| | | | | REQETPPDFFYFSDYERHNAEIAAFHLDR |
| | | | | ILDFRRVPPVAGRMVNMTKEIRDVTRDK |
| | | | | KLWRTFFISPANNICFYGECSYYCSTEHA |
| | | | | LCGKPDQIEGSLAAFLPDLSLAKRKTWR |
| | | | | NPWRRSYHKRKKAEWEVDPDYCEEVKQ |
| | | | | TPPYDSSHRILDVMDMTIFDFLMGNMDR |
| | | | | HHYETFEKFGNETFIIHLDNGRGFGKYSH |
| | | | | DELSILVPLQQCCRIRKSTYLRLQLLAKE |
| | | | | EYKLSLLMAESLRGDQVAPVLYQPHLEA |
| | | | | LDRRLRVVLKAVRDCVERNGLHSVVDD |
| | | | | DLDTEHRAASARDYKDDDDK |
| 111 | ScKRE2 | Fam20c | ScKRE(M | MALFLSKRLLRFTVIAGAVIVLLLTLNSN |
| | (M1-158) | (R64- | 1-158)_ | SRTQQYIPSSISAAFDFTSGSISPEQQVIRG |
| | | R584) | Fam20c | RPGEPPAASSAAGDAGWPNKHTLRILQD |
| | | | (R64- | FSSDPSSNLSSHSLEKLPPAAEPAERALRG |
| | | | R584)_FLAG | RDPGALRPHDPAHRPLLRDPGPRRSESPP |
| | | | | GPGGDASLLARLFEHPLYRVAVPPLTEE |
| | | | | DVLFNVNSDTRLSPKAAENPDWPHAGAE |
| | | | | GAEFLSPGEAAVDSYPNWLKFHIGINRYE |
| | | | | LYSRHNPAIEALLHDLSSQRITSVAMKSG |
| | | | | GTQLKLIMTFQNYGQALFKPMKQTREQ |
| | | | | ETPPDFFYFSDYERHNAEIAAFHLDRILDF |
| | | | | RRVPPVAGRMVNMTKEIRDVTRDKKLW |
| | | | | RTFFISPANNICFYGECSYYCSTEHALCG |
| | | | | KPDQIEGSLAAFLPDLSLAKRKTWRNPW |
| | | | | RRSYHKRKKAEWEVDPDYCEEVKQTPP |
| | | | | YDSSHRILDVMDMTIFDFLMGNMDRHH |
| | | | | YETFEKFGNETFIIHLDNGRGFGKYSHDE |
| | | | | LSILVPLQQCCRIRKSTYLRLQLLAKEEY |
| | | | | KLSLLMAESLRGDQVAPVLYQPHLEALD |
| | | | | RRLRVVLKAVRDCVERNGLHSVVDDDL |
| | | | | DTEHRAASARDYKDDDDK |
| 112 | ScKRE2 | Fam20c | ScKRE2( | MALFLSKRLLRFTVIAGAVIVLLLTLNSN |
| | (M1-S80) | (R64- | M1- | SRTQQYIPSSISAAFDFTSGSISPEQQVISE |
| | | R584) | S80)_Fam | ENDAKKLEQSALNSEASEDSRGRPGEPPA |
| | | | 20c(R64- | ASSAAGDAGWPNKHTLRILQDFSSDPSSN |
| | | | R584)_FLAG | LSSHSLEKLPPAAEPAERALRGRDPGALR |
| | | | | PHDPAHRPLLRDPGPRRSESPPGPGGDAS |
| | | | | LLARLFEHPLYRVAVPPLTEEDVLENVNS |
| | | | | DTRLSPKAAENPDWPHAGAEGAEFLSPG |
| | | | | EAAVDSYPNWLKFHIGINRYELYSRANPA |
| | | | | IEALLHDLSSQRITSVAMKSGGTQLKLIM |
| | | | | TFQNYGQALFKPMKQTREQETPPDFFYF |
| | | | | SDYERHNAEIAAFHLDRILDFRRVPPVAG |
| | | | | RMVNMTKEIRDVTRDKKLWRTFFISPAN |
| | | | | NICFYGECSYYCSTEHALCGKPDQIEGSL |
| | | | | AAFLPDLSLAKRKTWRNPWRRSYHKRK |
| | | | | KAEWEVDPDYCEEVKQTPPYDSSHRILD |
| | | | | VMDMTIFDFLMGNMDRHHYETFEKEGN |
| | | | | ETFIIHLDNGRGFGKYSHDELSILVPLQQ |
| | | | | CCRIRKSTYLRLQLLAKEEYKLSLLMAE |
| | | | | SLRGDQVAPVLYQPHLEALDRRLRVVLK |
| | | | | AVRDCVERNGLHSVVDDDLDTEHRAASA |
| | | | | RDYKDDDDK |
| 113 | ScKRE2 | Fam20c | ScKRE2( | MALFLSKRLLRFTVIAGAVIVLLLTLNSN |
| | (M1-D102) | (R64- | M1- | SRTQQYIPSSISAAFDFTSGSISPEQQVISE |
| | | R584) | D102)_Fa | ENDAKKLEQSALNSEASEDSEAMDEESK |
| | | | m20c(R64- | ALKAAAEKADAPIDRGRPGEPPAASSAA |
| | | | R584)_FLAG | GDAGWPNKHTLRILQDFSSDPSSNLSSHS |
| | | | | LEKLPPAAEPAERALRGRDPGALRPHDP |
| | | | | AHRPLLRDPGPRRSESPPGPGGDASLLAR |
| | | | | LFEHPLYRVAVPPLTEEDVLFNVNSDTRL |
| | | | | SPKAAENPDWPHAGAEGAEFLSPGEAAV |
| | | | | DSYPNWLKFHIGINRYELYSRHNPAIEAL |
| | | | | LHDLSSQRITSVAMKSGGTQLKLIMTFQ |
| | | | | NYGQALFKPMKQTREQETPPDFFYFSDY |
| | | | | ERHNAEIAAFHLDRILDFRRVPPVAGRM |
| | | | | VNMTKEIRDVTRDKKLWRTFFISPANNIC |
| | | | | FYGECSYYCSTEHALCGKPDQIEGSLAAF |
| | | | | LPDLSLAKRKTWRNPWRRSYHKRKKAE |
| | | | | WEVDPDYCEEVKQTPPYDSSHRILDVMD |
| | | | | MTIFDFLMGNMDRHHYETFEKFGNETFI |
| | | | | IHLDNGRGFGKYSHDELSILVPLQQCCRI |
| | | | | RKSTYLRLQLLAKEEYKLSLLMAESLRG |
| | | | | DQVAPVLYQPHLEALDRRLRVVLKAVRD |
| | | | | CVERNGLHSVVDDDLDTEHRAASARDYK |
| | | | | DDDDK |
| 114 | PpKRE2 | Fam20c | PpKRE2( | MVHIGFRSLKAVFILALSSLILYGIVTTFD |
| | (M1-G31) | (R64- | M1- | GRGRPGEPPAASSAAGDAGWPNKHTLRI |
| | | R584) | G31)_Fam | LQDFSSDPSSNLSSHSLEKLPPAAEPAERA |
| | | | 20c(R64- | LRGRDPGALRPHDPAHRPLLRDPGPRRS |
| | | | R584)_FLAG | ESPPGPGGDASLLARLFEHPLYRVAVPPL |
| | | | | TEEDVLFNVNSDTRLSPKAAENPDWPHA |
| | | | | GAEGAEFLSPGEAAVDSYPNWLKFHIGIN |
| | | | | RYELYSRHNPAIEALLHDLSSQRITSVAM |
| | | | | KSGGTQLKLIMTFQNYGQALFKPMKQT |
| | | | | REQETPPDFFYFSDYERHNAEIAAFHLDR |
| | | | | ILDFRRVPPVAGRMVNMTKEIRDVTRDK |
| | | | | KLWRTFFISPANNICFYGECSYYCSTEHA |
| | | | | LCGKPDQIEGSLAAFLPDLSLAKRKTWR |
| | | | | NPWRRSYHKRKKAEWEVDPDYCEEVKQ |
| | | | | TPPYDSSHRILDVMDMTIFDFLMGNMDR |
| | | | | HHYETFEKFGNETFIIHLDNGRGFGKYSH |
| | | | | DELSILVPLQQCCRIRKSTYLRLQLLAKE |
| | | | | EYKLSLLMAESLRGDQVAPVLYQPHLEA |
| | | | | LDRRLRVVLKAVRDCVERNGLHSVVDD |
| | | | | DLDTEHRAASARDYKDDDDK |
| 115 | PpKRE2 | Fam20c | PpKRE2( | MVHIGFRSLKAVFILALSSLILYGIVTTFD |
| | (M1-D84) | (R64- | M1- | GSRASRYQPPYVNHSQDPLYHSGNSYNR |
| | | R584) | D84)_Fam | ENATFVTLCRNEDLYSHIQSIKKVEDRGR |
| | | | 20c(R64- | PGEPPAASSAAGDAGWPNKHTLRILQDE |
| | | | R584)_FLAG | SSDPSSNLSSHSLEKLPPAAEPAERALRGR |
| | | | | DPGALRPHDPAHRPLLRDPGPRRSESPPG |
| | | | | PGGDASLLARLFEHPLYRVAVPPLTEEDV |
| | | | | LFNVNSDTRLSPKAAENPDWPHAGAEGA |
| | | | | EFLSPGEAAVDSYPNWLKFHIGINRYELY |
| | | | | SRHNPAIEALLHDLSSQRITSVAMKSGGT |
| | | | | QLKLIMTFQNYGQALFKPMKQTREQET |
| | | | | PPDFFYFSDYERHNAEIAAFHLDRILDFR |
| | | | | RVPPVAGRMVNMTKEIRDVTRDKKLWR |
| | | | | TFFISPANNICFYGECSYYCSTEHALCGKP |
| | | | | DQIEGSLAAFLPDLSLAKRKTWRNPWRR |
| | | | | SYHKRKKAEWEVDPDYCEEVKQTPPYDS |
| | | | | SHRILDVMDMTIFDFLMGNMDRHHYETF |
| | | | | EKFGNETFIIHLDNGRGFGKYSHDELSIL |
| | | | | VPLQQCCRIRKSTYLRLQLLAKEEYKLS |
| | | | | LLMAESLRGDQVAPVLYQPHLEALDRRL |
| | | | | RVVLKAVRDCVERNGLHSVVDDDLDTE |
| | | | | HRAASARDYKDDDDK |
| 116 | PpKRE2 | Fam20c | PpKRE2( | MVHIGFRSLKAVFILALSSLILYGIVTTED |
| | (M1-H150) | (R64- | M1- | GSRASRYQPPYVNHSQDPLYHSGNSYNR |
| | | R584) | H150)_Fa | ENATFVTLCRNEDLYSHIQSIKKVEDRENN |
| | | | m20c(R64- | KFAYDWVFLNEVPFTDEFKERTSVLISGQ |
| | | | R584)_FLAG | AKYGLIPKEHWSYPDYIDQERAAESRRQ |
| | | | | LEDQHRGRPGEPPAASSAAGDAGWPNK |
| | | | | HTLRILQDFSSDPSSNLSSHSLEKLPPAAE |
| | | | | PAERALRGRDPGALRPHDPAHRPLLRDP |
| | | | | GPRRSESPPGPGGDASLLARLFEHPLYRV |
| | | | | AVPPLTEEDVLFNVNSDTRLSPKAAENPD |
| | | | | WPHAGAEGAEFLSPGEAAVDSYPNWLKF |
| | | | | HIGINRYELYSRHNPAIEALLHDLSSQRIT |
| | | | | SVAMKSGGTQLKLIMTFQNYGQALFKP |
| | | | | MKQTREQETPPDFFYFSDYERHNAEIAAF |
| | | | | HLDRILDFRRVPPVAGRMVNMTKEIRDV |
| | | | | TRDKKLWRTFFISPANNICFYGECSYYCS |
| | | | | TEHALCGKPDQIEGSLAAFLPDLSLAKRK |
| | | | | TWRNPWRRSYHKRKKAEWEVDPDYCEE |
| | | | | VKQTPPYDSSHRILDVMDMTIFDFLMGN |
| | | | | MDRHHYETFEKFGNETFIHLDNGRGFG |
| | | | | KYSHDELSILVPLQQCCRIRKSTYLRLQL |
| | | | | LAKEEYKLSLLMAESLRGDQVAPVLYQP |
| | | | | HLEALDRRLRVVLKAVRDCVERNGLHSV |
| | | | | VDDDLDTEHRAASARDYKDDDDK |
| 117 | ScMNN2 | Fam20c | ScMNN2( | MLLTKRFSKLFKLTFIVLILCGLFVITNK |
| | (M1-S36) | (R64- | M1- | YMDENTSRGRPGEPPAASSAAGDAGWPN |
| | | R584) | S36)_Fam | KHTLRILQDFSSDPSSNLSSHSLEKLPPAA |
| | | | 20c(R64- | EPAERALRGRDPGALRPHDPAHRPLLRD |
| | | | R584)_FLAG | PGPRRSESPPGPGGDASLLARLFEHPLYR |
| | | | | VAVPPLTEEDVLFNVNSDTRLSPKAAENP |
| | | | | DWPHAGAEGAEFLSPGEAAVDSYPNWL |
| | | | | KFHIGINRYELYSRANPAIEALLHDLSSQ |
| | | | | RITSVAMKSGGTQLKLIMTFQNYGQALF |
| | | | | KPMKQTREQETPPDFFYFSDYERHNAEI |
| | | | | AAFHLDRILDFRRVPPVAGRMVNMTKEI |
| | | | | RDVTRDKKLWRTFFISPANNICFYGECSY |
| | | | | YCSTEHALCGKPDQIEGSLAAFLPDLSLA |
| | | | | KRKTWRNPWRRSYHKRKKAEWEVDPD |
| | | | | YCEEVKQTPPYDSSHRILDVMDMTIFDFL |
| | | | | MGNMDRHHYETFEKFGNETFIIHLDNGR |
| | | | | GFGKYSHDELSILVPLQQCCRIRKSTYLR |
| | | | | LQLLAKEEYKLSLLMAESLRGDQVAPVL |
| | | | | YQPHLEALDRRLRVVLKAVRDCVERNG |
| | | | | LHSVVDDDLDTEHRAASARDYKDDDDK |
| 118 | ScMNN2 | Fam20c | ScMNN2( | MLLTKRFSKLFKLTFIVLILCGLFVITNK |
| | (M1-P97) | (R64- | M1- | YMDENTSVKEYKEYLDRYVQSYSNKYSS |
| | | R584) | P97)_Fam | SSDAASADDSTPLRDNDEAGNEKLKSFYN |
| | | | 20c(R64- | NVFNFLMVDSPRGRPGEPPAASSAAGDA |
| | | | R584)_FLAG | GWPNKHTLRILQDFSSDPSSNLSSHSLEK |
| | | | | LPPAAEPAERALRGRDPGALRPHDPAHR |
| | | | | PLLRDPGPRRSESPPGPGGDASLLARLFE |
| | | | | HPLYRVAVPPLTEEDVLFNVNSDTRLSPK |
| | | | | AAENPDWPHAGAEGAEFLSPGEAAVDSY |
| | | | | PNWLKFHIGINRYELYSRHNPAIEALLHD |
| | | | | LSSQRITSVAMKSGGTQLKLIMTFQNYG |
| | | | | QALFKPMKQTREQETPPDFFYFSDYERH |
| | | | | NAEIAAFHLDRILDFRRVPPVAGRMVNM |
| | | | | TKEIRDVTRDKKLWRTFFISPANNICFYG |
| | | | | ECSYYCSTEHALCGKPDQIEGSLAAFLPD |
| | | | | LSLAKRKTWRNPWRRSYHKRKKAEWE |
| | | | | VDPDYCEEVKQTPPYDSSHRILDVMDMT |
| | | | | IFDFLMGNMDRHHYETFEKFGNETFIHL |
| | | | | DNGRGFGKYSHDELSILVPLQQCCRIRKS |
| | | | | TYLRLQLLAKEEYKLSLLMAESLRGDQV |
| | | | | APVLYQPHLEALDRRLRVVLKAVRDCVE |
| | | | | RNGLHSVVDDDLDTEHRAASARDYKDDD |
| | | | | DK |
| 119 | ScMNN2 | Fam20c | ScMNN2( | MLLTKRFSKLFKLTFIVLILCGLFVITNK |
| | (M1-S150) | (R64- | M1-S150)_ | YMDENTSVKEYKEYLDRYVQSYSNKYSS |
| | | R584) | Fam20c | SSDAASADDSTPLRDNDEAGNEKLKSFYN |
| | | | (R64- | NVFNFLMVDSPKGSTAKQYNEACLLKGD |
| | | | R584)_FLAG | IGDRPDHYKDLYKLSAKELSKCLELSPDE |
| | | | | VASLTKSRGRPGEPPAASSAAGDAGWPN |
| | | | | KHTLRILQDFSSDPSSNLSSHSLEKLPPAA |
| | | | | EPAERALRGRDPGALRPHDPAHRPLLRD |
| | | | | PGPRRSESPPGPGGDASLLARLFEHPLYR |
| | | | | VAVPPLTEEDVLFNVNSDTRLSPKAAENP |
| | | | | DWPHAGAEGAEFLSPGEAAVDSYPNWL |
| | | | | KFHIGINRYELYSRANPAIEALLHDLSSQ |
| | | | | RITSVAMKSGGTQLKLIMTFQNYGQALF |
| | | | | KPMKQTREQETPPDFFYFSDYERHNAEI |
| | | | | AAFHLDRILDFRRVPPVAGRMVNMTKEI |
| | | | | RDVTRDKKLWRTFFISPANNICFYGECSY |
| | | | | YCSTEHALCGKPDQIEGSLAAFLPDLSLA |
| | | | | KRKTWRNPWRRSYHKRKKAEWEVDPD |
| | | | | YCEEVKQTPPYDSSHRILDVMDMTIFDFL |
| | | | | MGNMDRHHYETFEKFGNETFIIHLDNGR |
| | | | | GFGKYSHDELSILVPLQQCCRIRKSTYLR |
| | | | | LQLLAKEEYKLSLLMAESLRGDQVAPVL |
| | | | | YQPHLEALDRRLRVVLKAVRDCVERNG |
| | | | | LHSVVDDDLDTEHRAASARDYKDDDDK |
| 120 | ScMNN1 | Fam20c | ScMNN1( | MLALRRFILNQRSLRSCTIPILVGALIILV |
| | (M1-A42) | (R64- | M1- | LFQLVTHRNDARGRPGEPPAASSAAGDA |
| | | R584) | A42)_Fam | GWPNKHTLRILQDFSSDPSSNLSSHSLEK |
| | | | 20c(R64- | LPPAAEPAERALRGRDPGALRPHDPAHR |
| | | | R584)_FLAG | PLLRDPGPRRSESPPGPGGDASLLARLFE |
| | | | | HPLYRVAVPPLTEEDVLFNVNSDTRLSPK |
| | | | | AAENPDWPHAGAEGAEFLSPGEAAVDSY |
| | | | | PNWLKFHIGINRYELYSRHNPAIEALLHD |
| | | | | LSSQRITSVAMKSGGTQLKLIMTFQNYG |
| | | | | QALFKPMKQTREQETPPDFFYFSDYERH |
| | | | | NAEIAAFHLDRILDFRRVPPVAGRMVNM |
| | | | | TKEIRDVTRDKKLWRTFFISPANNICFYG |
| | | | | ECSYYCSTEHALCGKPDQIEGSLAAFLPD |
| | | | | LSLAKRKTWRNPWRRSYHKRKKAEWE |
| | | | | VDPDYCEEVKQTPPYDSSHRILDVMDMT |
| | | | | IFDFLMGNMDRHHYETFEKFGNETFUIHL |
| | | | | DNGRGFGKYSHDELSILVPLQQCCRIRKS |
| | | | | TYLRLQLLAKEEYKLSLLMAESLRGDQV |
| | | | | APVLYQPHLEALDRRLRVVLKAVRDCVE |
| | | | | RNGLHSVVDDDLDTEHRAASARDYKDDD |
| | | | | DK |
| 121 | ScMNN1 | Fam20c | ScMNN1( | MLALRRFILNQRSLRSCTIPILVGALIIILV |
| | (M1-Q93) | (R64- | M1-Q93)_ | LFQLVTHRNDALIRSSNVNSTNKKTLKDA |
| | | R584) | Fam | DPKVLIEAFGSPEVDPVDTIPVSPLELVPF |
| | | | 20c(R64- | YDQRGRPGEPPAASSAAGDAGWPNKHT |
| | | | R584)_FLAG | LRILQDFSSDPSSNLSSHSLEKLPPAAEPA |
| | | | | ERALRGRDPGALRPHDPAHRPLLRDPGP |
| | | | | RRSESPPGPGGDASLLARLFEHPLYRVAV |
| | | | | PPLTEEDVLFNVNSDTRLSPKAAENPDWP |
| | | | | HAGAEGAEFLSPGEAAVDSYPNWLKFHI |
| | | | | GINRYELYSRHNPAIEALLHDLSSQRITSV |
| | | | | AMKSGGTQLKLIMTFQNYGQALFKPMK |
| | | | | QTREQETPPDFFYFSDYERHNAEIAAFHL |
| | | | | DRILDFRRVPPVAGRMVNMTKEIRDVTR |
| | | | | DKKLWRTFFISPANNICFYGECSYYCSTE |
| | | | | HALCGKPDQIEGSLAAFLPDLSLAKRKT |
| | | | | WRNPWRRSYHKRKKAEWEVDPDYCEE |
| | | | | VKQTPPYDSSHRILDVMDMTIFDFLMGN |
| | | | | MDRHHYETFEKFGNETFIHLDNGRGFG |
| | | | | KYSHDELSILVPLQQCCRIRKSTYLRLQL |
| | | | | LAKEEYKLSLLMAESLRGDQVAPVLYQP |
| | | | | HLEALDRRLRVVLKAVRDCVERNGLHSV |
| | | | | VDDDLDTEHRAASARDYKDDDDK |
| 122 | ScMINN1 | Fam20c | ScMNN1( | MLALRRFILNQRSLRSCTIPILVGALIILV |
| | (M1-G153) | (R64- | M1-G153)_ | LFQLVTHRNDALIRSSNVNSTNKKTLKDA |
| | | R584) | Fam20c | DPKVLIEAFGSPEVDPVDTIPVSPLELVPF |
| | | | (R64- | YDQSIDTKRSSSWLINKKGYYKHFNELSL |
| | | | R584)_FLAG | TDRCKFYFRTLYTLDDEWTNSVKKLEYS |
| | | | | INDNEGRGRPGEPPAASSAAGDAGWPNK |
| | | | | HTLRILQDFSSDPSSNLSSHSLEKLPPAAE |
| | | | | PAERALRGRDPGALRPHDPAHRPLLRDP |
| | | | | GPRRSESPPGPGGDASLLARLFEHPLYRV |
| | | | | AVPPLTEEDVLFNVNSDTRLSPKAAENPD |
| | | | | WPHAGAEGAEFLSPGEAAVDSYPNWLKF |
| | | | | HIGINRYELYSRHNPAIEALLHDLSSQRIT |
| | | | | SVAMKSGGTQLKLIMTFQNYGQALFKP |
| | | | | MKQTREQETPPDFFYFSDYERHNAEIAAF |
| | | | | HLDRILDFRRVPPVAGRMVNMTKEIRDV |
| | | | | TRDKKLWRTFFISPANNICFYGECSYYCS |
| | | | | TEHALCGKPDQIEGSLAAFLPDLSLAKRK |
| | | | | TWRNPWRRSYHKRKKAEWEVDPDYCEE |
| | | | | VKQTPPYDSSHRILDVMDMTIFDFLMGN |
| | | | | MDRHHYETFEKFGNETFIIHLDNGRGFG |
| | | | | KYSHDELSILVPLQQCCRIRKSTYLRLQL |
| | | | | LAKEEYKLSLLMAESLRGDQVAPVLYQP |
| | | | | HLEALDRRLRVVLKAVRDCVERNGLHSV |
| | | | | VDDDLDTEHRAASARDYKDDDDK |
| 123 | ScMINN6 | Fam20c | ScMNN6( | MHVLLSKKIARFLLISFVFVLALMVTINH |
| | (M1-P30) | (R64- | M1-P30)_ | PRGRPGEPPAASSAAGDAGWPNKHTLRI |
| | | R584) | Fam | LQDFSSDPSSNLSSHSLEKLPPAAEPAERA |
| | | | 20c(R64- | LRGRDPGALRPHDPAHRPLLRDPGPRRS |
| | | | R584)_FLAG | ESPPGPGGDASLLARLFEHPLYRVAVPPL |
| | | | | TEEDVLENVNSDTRLSPKAAENPDWPHA |
| | | | | GAEGAEFLSPGEAAVDSYPNWLKFHIGIN |
| | | | | RYELYSRHNPAIEALLHDLSSQRITSVAM |
| | | | | KSGGTQLKLIMTFQNYGQALFKPMKQT |
| | | | | REQETPPDFFYFSDYERHNAEIAAFHLDR |
| | | | | ILDFRRVPPVAGRMVNMTKEIRDVTRDK |
| | | | | KLWRTFFISPANNICFYGECSYYCSTEHA |
| | | | | LCGKPDQIEGSLAAFLPDLSLAKRKTWR |
| | | | | NPWRRSYHKRKKAEWEVDPDYCEEVKQ |
| | | | | TPPYDSSHRILDVMDMTIFDFLMGNMDR |
| | | | | HHYETFEKFGNETFIIHLDNGRGFGKYSH |
| | | | | DELSILVPLQQCCRIRKSTYLRLQLLAKE |
| | | | | EYKLSLLMAESLRGDQVAPVLYQPHLEA |
| | | | | LDRRLRVVLKAVRDCVERNGLHSVVDD |
| | | | | DLDTEHRAASARDYKDDDDK |
| 124 | ScMNN6 | Fam20c | ScMNN6 | MHVLLSKKIARFLLISFVFVLALMVTINH |
| | (M1-V85) | (R64- | (M1-V85)_ | PKTKQMSEQYVTPYLPKSLQPIAKISAEE |
| | | R584) | Fam20c | QRRIQSEQEEAELKQSLEGEAIRNATVRG |
| | | | (R64- | RPGEPPAASSAAGDAGWPNKHTLRILQD |
| | | | R584)_FLAG | FSSDPSSNLSSHSLEKLPPAAEPAERALRG |
| | | | | RDPGALRPHDPAHRPLLRDPGPRRSESPP |
| | | | | GPGGDASLLARLFEHPLYRVAVPPLTEE |
| | | | | DVLFNVNSDTRLSPKAAENPDWPHAGAE |
| | | | | GAEFLSPGEAAVDSYPNWLKFHIGINRYE |
| | | | | LYSRHNPAIEALLHDLSSQRITSVAMKSG |
| | | | | GTQLKLIMTFQNYGQALFKPMKQTREQ |
| | | | | ETPPDFFYFSDYERHNAEIAAFHLDRILDF |
| | | | | RRVPPVAGRMVNMTKEIRDVTRDKKLW |
| | | | | RTFFISPANNICFYGECSYYCSTEHALCG |
| | | | | KPDQIEGSLAAFLPDLSLAKRKTWRNPW |
| | | | | RRSYHKRKKAEWEVDPDYCEEVKQTPP |
| | | | | YDSSHRILDVMDMTIFDFLMGNMDRHH |
| | | | | YETFEKFGNETFIHLDNGRGFGKYSHDE |
| | | | | LSILVPLQQCCRIRKSTYLRLQLLAKEEY |
| | | | | KLSLLMAESLRGDQVAPVLYQPHLEALD |
| | | | | RRLRVVLKAVRDCVERNGLHSVVDDDL |
| | | | | DTEHRAASARDYKDDDDK |
| 125 | ScMNN6 | Fam20c | ScMNN6( | MHVLLSKKIARFLLISFVFVLALMVTINH |
| | (M1-E160) | (R64- | M1- | PKTKQMSEQYVTPYLPKSLQPIAKISAEE |
| | | R584) | E160)_Fa | QRRIQSEQEEAELKQSLEGEAIRNATVNA |
| | | | m20c(R64- | IKEKIKSYGGNETTLGFMVPSYINHRGSP |
| | | | R584)_FLAG | PKACFVSLITERDSMTQILQSIDEVQVKF |
| | | | | NKNFAYPWVFISQGERGRPGEPPAASSAA |
| | | | | GDAGWPNKHTLRILQDFSSDPSSNLSSHS |
| | | | | LEKLPPAAEPAERALRGRDPGALRPHDP |
| | | | | AHRPLLRDPGPRRSESPPGPGGDASLLAR |
| | | | | LFEHPLYRVAVPPLTEEDVLFNVNSDTRL |
| | | | | SPKAAENPDWPHAGAEGAEFLSPGEAAV |
| | | | | DSYPNWLKFHIGINRYELYSRHNPAIEAL |
| | | | | LHDLSSQRITSVAMKSGGTQLKLIMTFQ |
| | | | | NYGQALFKPMKQTREQETPPDFFYFSDY |
| | | | | ERHNAEIAAFHLDRILDFRRVPPVAGRM |
| | | | | VNMTKEIRDVTRDKKLWRTFFISPANNIC |
| | | | | FYGECSYYCSTEHALCGKPDQIEGSLAAF |
| | | | | LPDLSLAKRKTWRNPWRRSYHKRKKAE |
| | | | | WEVDPDYCEEVKQTPPYDSSHRILDVMD |
| | | | | MTIFDFLMGNMDRHHYETFEKFGNETFI |
| | | | | IHLDNGRGFGKYSHDELSILVPLQQCCRI |
| | | | | RKSTYLRLQLLAKEEYKLSLLMAESLRG |
| | | | | DQVAPVLYQPHLEALDRRLRVVLKAVRD |
| | | | | CVERNGLHSVVDDDLDTEHRAASARDYK |
| | | | | DDDDK |
| 126 | N/A | Fam20c | Fam20c(D | MDFSSDPSSNLSSHSLEKLPPAAEPAERAL |
| | | (D93- | 93- | RGRDPGALRPHDPAHRPLLRDPGPRRSE |
| | | R584) | R584)_FLAG | SPPGPGGDASLLARLFEHPLYRVAVPPLT |
| | | | | EEDVLFNVNSDTRLSPKAAENPDWPHAG |
| | | | | AEGAEFLSPGEAAVDSYPNWLKFHIGINR |
| | | | | YELYSRHNPAIEALLHDLSSQRITSVAMK |
| | | | | SGGTQLKLIMTFQNYGQALFKPMKQTR |
| | | | | EQETPPDFFYFSDYERHNAEIAAFHLDRI |
| | | | | LDFRRVPPVAGRMVNMTKEIRDVTRDK |
| | | | | KLWRTFFISPANNICFYGECSYYCSTEHA |
| | | | | LCGKPDQIEGSLAAFLPDLSLAKRKTWR |
| | | | | NPWRRSYHKRKKAEWEVDPDYCEEVKQ |
| | | | | TPPYDSSHRILDVMDMTIFDFLMGNMDR |
| | | | | HHYETFEKFGNETFIIHLDNGRGFGKYSH |
| | | | | DELSILVPLQQCCRIRKSTYLRLQLLAKE |
| | | | | EYKLSLLMAESLRGDQVAPVLYQPHLEA |
| | | | | LDRRLRVVLKAVRDCVERNGLHSVVDD |
| | | | | DLDTEHRAASARDYKDDDDK |
| 127 | ScKRE2 | Fam20c | ScKRE(M | MALFLSKRLLRFTVIAGAVIVLLLTLNSN |
| | (M1-158) | (D93- | 1-158)_ | SRTQQYIPSSISAAFDFTSGSISPEQQVIDF |
| | | R584) | Fam20c | SSDPSSNLSSHSLEKLPPAAEPAERALRGR |
| | | | (D93- | DPGALRPHDPAHRPLLRDPGPRRSESPPG |
| | | | R584)_FLAG | PGGDASLLARLFEHPLYRVAVPPLTEEDV |
| | | | | LFNVNSDTRLSPKAAENPDWPHAGAEGA |
| | | | | EFLSPGEAAVDSYPNWLKFHIGINRYELY |
| | | | | SRHNPAIEALLHDLSSQRITSVAMKSGGT |
| | | | | QLKLIMTFQNYGQALFKPMKQTREQET |
| | | | | PPDFFYFSDYERHNAEIAAFHLDRILDER |
| | | | | RVPPVAGRMVNMTKEIRDVTRDKKLWR |
| | | | | TFFISPANNICFYGECSYYCSTEHALCGKP |
| | | | | DQIEGSLAAFLPDLSLAKRKTWRNPWRR |
| | | | | SYHKRKKAEWEVDPDYCEEVKQTPPYDS |
| | | | | SHRILDVMDMTIFDFLMGNMDRHHYETF |
| | | | | EKFGNETFIIHLDNGRGFGKYSHDELSIL |
| | | | | VPLQQCCRIRKSTYLRLQLLAKEEYKLS |
| | | | | LLMAESLRGDQVAPVLYQPHLEALDRRL |
| | | | | RVVLKAVRDCVERNGLHSVVDDDLDTE |
| | | | | HRAASARDYKDDDDK |
| 128 | ScKRE2 | Fam20c | ScKRE2( | MALFLSKRLLRFTVIAGAVIVLLLTLNSN |
| | (M1-S80) | (D93- | M1-S80)_ | SRTQQYIPSSISAAFDFTSGSISPEQQVISE |
| | | R584) | Fam20c | ENDAKKLEQSALNSEASEDSDFSSDPSSNL |
| | | | (D93- | SSHSLEKLPPAAEPAERALRGRDPGALRP |
| | | | R584)_FLAG | HDPAHRPLLRDPGPRRSESPPGPGGDASL |
| | | | | LARLFEHPLYRVAVPPLTEEDVLFNVNSD |
| | | | | TRLSPKAAENPDWPHAGAEGAEFLSPGE |
| | | | | AAVDSYPNWLKFHIGINRYELYSRHNPAI |
| | | | | EALLHDLSSQRITSVAMKSGGTQLKLIM |
| | | | | TFQNYGQALFKPMKQTREQETPPDFFYF |
| | | | | SDYERHNAEIAAFHLDRILDFRRVPPVAG |
| | | | | RMVNMTKEIRDVTRDKKLWRTFFISPAN |
| | | | | NICFYGECSYYCSTEHALCGKPDQIEGSL |
| | | | | AAFLPDLSLAKRKTWRNPWRRSYHKRK |
| | | | | KAEWEVDPDYCEEVKQTPPYDSSHRILD |
| | | | | VMDMTIFDFLMGNMDRHHYETFEKEGN |
| | | | | ETFIIHLDNGRGFGKYSHDELSILVPLQQ |
| | | | | CCRIRKSTYLRLQLLAKEEYKLSLLMAE |
| | | | | SLRGDQVAPVLYQPHLEALDRRLRVVLK |
| | | | | AVRDCVERNGLHSVVDDDLDTEHRAASA |
| | | | | RDYKDDDDK |
| 129 | ScKRE2 | Fam20c | ScKRE2( | MALFLSKRLLRFTVIAGAVIVLLLTLNSN |
| | (M1-D102) | (D93- | M1-D102)_ | SRTQQYIPSSISAAFDFTSGSISPEQQVISE |
| | | R584) | Fam20c | ENDAKKLEQSALNSEASEDSEAMDEESK |
| | | | (D93- | ALKAAAEKADAPIDDESSDPSSNLSSHSLE |
| | | | R584)_FLAG | KLPPAAEPAERALRGRDPGALRPHDPAH |
| | | | | RPLLRDPGPRRSESPPGPGGDASLLARLF |
| | | | | EHPLYRVAVPPLTEEDVLFNVNSDTRLSP |
| | | | | KAAENPDWPHAGAEGAEFLSPGEAAVDS |
| | | | | YPNWLKFHIGINRYELYSRANPAIEALLH |
| | | | | DLSSQRITSVAMKSGGTQLKLIMTFQNY |
| | | | | GQALFKPMKQTREQETPPDFFYFSDYER |
| | | | | HNAEIAAFHLDRILDFRRVPPVAGRMVN |
| | | | | MTKEIRDVTRDKKLWRTFFISPANNICFY |
| | | | | GECSYYCSTEHALCGKPDQIEGSLAAFLP |
| | | | | DLSLAKRKTWRNPWRRSYHKRKKAEW |
| | | | | EVDPDYCEEVKQTPPYDSSHRILDVMDM |
| | | | | TIFDFLMGNMDRHHYETFEKFGNETFIIH |
| | | | | LDNGRGFGKYSHDELSILVPLQQCCRIRK |
| | | | | STYLRLQLLAKEEYKLSLLMAESLRGDQ |
| | | | | VAPVLYQPHLEALDRRLRVVLKAVRDCV |
| | | | | ERNGLHSVVDDDLDTEHRAASARDYKDD |
| | | | | DDK |
| 130 | PpKRE2 | Fam20c | PpKRE2 | MVHIGFRSLKAVFILALSSLILYGIVTTFD |
| | (M1-G31) | (D93- | (M1-G31)_ | GDFSSDPSSNLSSHSLEKLPPAAEPAERAL |
| | | R584) | Fam20c | RGRDPGALRPHDPAHRPLLRDPGPRRSE |
| | | | (D93- | SPPGPGGDASLLARLFEHPLYRVAVPPLT |
| | | | R584)_FLAG | EEDVLFNVNSDTRLSPKAAENPDWPHAG |
| | | | | AEGAEFLSPGEAAVDSYPNWLKFHIGINR |
| | | | | YELYSRHNPAIEALLHDLSSQRITSVAMK |
| | | | | SGGTQLKLIMTFQNYGQALFKPMKQTR |
| | | | | EQETPPDFFYFSDYERHNAEIAAFHLDRI |
| | | | | LDFRRVPPVAGRMVNMTKEIRDVTRDK |
| | | | | KLWRTFFISPANNICFYGECSYYCSTEHA |
| | | | | LCGKPDQIEGSLAAFLPDLSLAKRKTWR |
| | | | | NPWRRSYHKRKKAEWEVDPDYCEEVKQ |
| | | | | TPPYDSSHRILDVMDMTIFDFLMGNMDR |
| | | | | HHYETFEKFGNETFIIHLDNGRGFGKYSH |
| | | | | DELSILVPLQQCCRIRKSTYLRLQLLAKE |
| | | | | EYKLSLLMAESLRGDQVAPVLYQPHLEA |
| | | | | LDRRLRVVLKAVRDCVERNGLHSVVDD |
| | | | | DLDTEHRAASARDYKDDDDK |
| 131 | PpKRE2 | Fam20c | PpKRE2 | MVHIGFRSLKAVFILALSSLILYGIVTTFD |
| | (M1-D84) | (D93- | (M1-D84)_ | GSRASRYQPPYVNHSQDPLYHSGNSYNR |
| | | R584) | Fam20c | ENATFVTLCRNEDLYSIIQSIKKVEDDFSS |
| | | | (D93- | DPSSNLSSHSLEKLPPAAEPAERALRGRD |
| | | | R584)_FLAG | PGALRPHDPAHRPLLRDPGPRRSESPPGP |
| | | | | GGDASLLARLFEHPLYRVAVPPLTEEDV |
| | | | | LFNVNSDTRLSPKAAENPDWPHAGAEGA |
| | | | | EFLSPGEAAVDSYPNWLKFHIGINRYELY |
| | | | | SRHNPAIEALLHDLSSQRITSVAMKSGGT |
| | | | | QLKLIMTFQNYGQALFKPMKQTREQET |
| | | | | PPDFFYFSDYERHNAEIAAFHLDRILDFR |
| | | | | RVPPVAGRMVNMTKEIRDVTRDKKLWR |
| | | | | TFFISPANNICFYGECSYYCSTEHALCGKP |
| | | | | DQIEGSLAAFLPDLSLAKRKTWRNPWRR |
| | | | | SYHKRKKAEWEVDPDYCEEVKQTPPYDS |
| | | | | SHRILDVMDMTIFDFLMGNMDRHHYETF |
| | | | | EKFGNETFIIHLDNGRGFGKYSHDELSIL |
| | | | | VPLQQCCRIRKSTYLRLQLLAKEEYKLS |
| | | | | LLMAESLRGDQVAPVLYQPHLEALDRRL |
| | | | | RVVLKAVRDCVERNGLHSVVDDDLDTE |
| | | | | HRAASARDYKDDDDK |
| 132 | PpKRE2 | Fam20c | PpKRE2( | MVHIGFRSLKAVFILALSSLILYGIVTTFD |
| | (M1-H150) | (D93- | M1- | GSRASRYQPPYVNHSQDPLYHSGNSYNR |
| | | R584) | H150)_Fa | ENATFVTLCRNEDLYSHIQSIKKVEDRENN |
| | | | m20c(D93- | KFAYDWVFLNEVPFTDEFKERTSVLISGQ |
| | | | R584)_FLAG | AKYGLIPKEHWSYPDYIDQERAAESRRQ |
| | | | | LEDQHDFSSDPSSNLSSHSLEKLPPAAEPA |
| | | | | ERALRGRDPGALRPHDPAHRPLLRDPGP |
| | | | | RRSESPPGPGGDASLLARLFEHPLYRVAV |
| | | | | PPLTEEDVLFNVNSDTRLSPKAAENPDWP |
| | | | | HAGAEGAEFLSPGEAAVDSYPNWLKFHI |
| | | | | GINRYELYSRANPAIEALLHDLSSQRITSV |
| | | | | AMKSGGTQLKLIMTFQNYGQALFKPMK |
| | | | | QTREQETPPDFFYFSDYERHNAEIAAFHL |
| | | | | DRILDFRRVPPVAGRMVNMTKEIRDVTR |
| | | | | DKKLWRTFFISPANNICFYGECSYYCSTE |
| | | | | HALCGKPDQIEGSLAAFLPDLSLAKRKT |
| | | | | WRNPWRRSYHKRKKAEWEVDPDYCEE |
| | | | | VKQTPPYDSSHRILDVMDMTIFDFLMGN |
| | | | | MDRHHYETFEKFGNETFIIHLDNGRGFG |
| | | | | KYSHDELSILVPLQQCCRIRKSTYLRLQL |
| | | | | LAKEEYKLSLLMAESLRGDQVAPVLYQP |
| | | | | HLEALDRRLRVVLKAVRDCVERNGLHSV |
| | | | | VDDDLDTEHRAASARDYKDDDDK |
| 133 | ScMNN2 | Fam20c | ScMNN2 | MLLTKRFSKLFKLTFIVLILCGLFVITNK |
| | (M1-S36) | (D93- | (M1-S36)_ | YMDENTSDFSSDPSSNLSSHSLEKLPPAAE |
| | | R584) | Fam20c | PAERALRGRDPGALRPHDPAHRPLLRDP |
| | | | (D93- | GPRRSESPPGPGGDASLLARLFEHPLYRV |
| | | | R584)_FLAG | AVPPLTEEDVLFNVNSDTRLSPKAAENPD |
| | | | | WPHAGAEGAEFLSPGEAAVDSYPNWLKF |
| | | | | HIGINRYELYSRHNPAIEALLHDLSSQRIT |
| | | | | SVAMKSGGTQLKLIMTFQNYGQALFKP |
| | | | | MKQTREQETPPDFFYFSDYERHNAEIAAF |
| | | | | HLDRILDFRRVPPVAGRMVNMTKEIRDV |
| | | | | TRDKKLWRTFFISPANNICFYGECSYYCS |
| | | | | TEHALCGKPDQIEGSLAAFLPDLSLAKRK |
| | | | | TWRNPWRRSYHKRKKAEWEVDPDYCEE |
| | | | | VKQTPPYDSSHRILDVMDMTIFDFLMGN |
| | | | | MDRHHYETFEKFGNETFIIHLDNGRGFG |
| | | | | KYSHDELSILVPLQQCCRIRKSTYLRLQL |
| | | | | LAKEEYKLSLLMAESLRGDQVAPVLYQP |
| | | | | HLEALDRRLRVVLKAVRDCVERNGLHSV |
| | | | | VDDDLDTEHRAASARDYKDDDDK |
| 134 | ScMNN2 | Fam20c | ScMNN2 | MLLTKRFSKLFKLTFIVLILCGLFVITNK |
| | (M1-P97) | (D93- | (M1-P97)_ | YMDENTSVKEYKEYLDRYVQSYSNKYSS |
| | | R584) | Fam20c( | SSDAASADDSTPLRDNDEAGNEKLKSFYN |
| | | | D93- | NVFNFLMVDSPDFSSDPSSNLSSHSLEKLP |
| | | | R584)_FLAG | PAAEPAERALRGRDPGALRPHDPAHRPL |
| | | | | LRDPGPRRSESPPGPGGDASLLARLFEHP |
| | | | | LYRVAVPPLTEEDVLFNVNSDTRLSPKAA |
| | | | | ENPDWPHAGAEGAEFLSPGEAAVDSYPN |
| | | | | WLKFHIGINRYELYSRANPAIEALLHDLS |
| | | | | SQRITSVAMKSGGTQLKLIMTFQNYGQA |
| | | | | LFKPMKQTREQETPPDFFYFSDYERHNA |
| | | | | EIAAFHLDRILDFRRVPPVAGRMVNMTK |
| | | | | EIRDVTRDKKLWRTFFISPANNICFYGEC |
| | | | | SYYCSTEHALCGKPDQIEGSLAAFLPDLS |
| | | | | LAKRKTWRNPWRRSYHKRKKAEWEVD |
| | | | | PDYCEEVKQTPPYDSSHRILDVMDMTIFD |
| | | | | FLMGNMDRHHYETFEKFGNETFIIHLDN |
| | | | | GRGFGKYSHDELSILVPLQQCCRIRKSTY |
| | | | | LRLQLLAKEEYKLSLLMAESLRGDQVAP |
| | | | | VLYQPHLEALDRRLRVVLKAVRDCVERN |
| | | | | GLHSVVDDDLDTEHRAASARDYKDDDD |
| | | | | K |
| 135 | ScMNN2 | Fam20c | ScMNN2( | MLLTKRFSKLFKLTFIVLILCGLFVITNK |
| | (M1-S150) | (D93- | M1- | YMDENTSVKEYKEYLDRYVQSYSNKYSS |
| | | R584) | S150)_Fa | SSDAASADDSTPLRDNDEAGNEKLKSFYN |
| | | | m20c(D93- | NVFNFLMVDSPKGSTAKQYNEACLLKGD |
| | | | R584)_FLAG | IGDRPDHYKDLYKLSAKELSKCLELSPDE |
| | | | | VASLTKSDFSSDPSSNLSSHSLEKLPPAAE |
| | | | | PAERALRGRDPGALRPHDPAHRPLLRDP |
| | | | | GPRRSESPPGPGGDASLLARLFEHPLYRV |
| | | | | AVPPLTEEDVLFNVNSDTRLSPKAAENPD |
| | | | | WPHAGAEGAEFLSPGEAAVDSYPNWLKF |
| | | | | HIGINRYELYSRHNPAIEALLHDLSSQRIT |
| | | | | SVAMKSGGTQLKLIMTFQNYGQALFKP |
| | | | | MKQTREQETPPDFFYFSDYERHNAEIAAF |
| | | | | HLDRILDFRRVPPVAGRMVNMTKEIRDV |
| | | | | TRDKKLWRTFFISPANNICFYGECSYYCS |
| | | | | TEHALCGKPDQIEGSLAAFLPDLSLAKRK |
| | | | | TWRNPWRRSYHKRKKAEWEVDPDYCEE |
| | | | | VKQTPPYDSSHRILDVMDMTIFDFLMGN |
| | | | | MDRHHYETFEKFGNETFIIHLDNGRGFG |
| | | | | KYSHDELSILVPLQQCCRIRKSTYLRLQL |
| | | | | LAKEEYKLSLLMAESLRGDQVAPVLYQP |
| | | | | HLEALDRRLRVVLKAVRDCVERNGLHSV |
| | | | | VDDDLDTEHRAASARDYKDDDDK |
| 136 | ScMNN1 | Fam20c | ScMNN1 | MLALRRFILNQRSLRSCTIPILVGALIILV |
| | (M1-A42) | (D93- | (M1-A42)_ | LFQLVTHRNDADFSSDPSSNLSSHSLEKLP |
| | | R584) | Fam20c | PAAEPAERALRGRDPGALRPHDPAHRPL |
| | | | (D93- | LRDPGPRRSESPPGPGGDASLLARLFEHP |
| | | | R584)_FLAG | LYRVAVPPLTEEDVLFNVNSDTRLSPKAA |
| | | | | ENPDWPHAGAEGAEFLSPGEAAVDSYPN |
| | | | | WLKFHIGINRYELYSRUNPAIEALLHDLS |
| | | | | SQRITSVAMKSGGTQLKLIMTFQNYGQA |
| | | | | LFKPMKQTREQETPPDFFYFSDYERHNA |
| | | | | EIAAFHLDRILDFRRVPPVAGRMVNMTK |
| | | | | EIRDVTRDKKLWRTFFISPANNICFYGEC |
| | | | | SYYCSTEHALCGKPDQIEGSLAAFLPDLS |
| | | | | LAKRKTWRNPWRRSYHKRKKAEWEVD |
| | | | | PDYCEEVKQTPPYDSSHRILDVMDMTIFD |
| | | | | FLMGNMDRHHYETFEKFGNETFIIHLDN |
| | | | | GRGFGKYSHDELSILVPLQQCCRIRKSTY |
| | | | | LRLQLLAKEEYKLSLLMAESLRGDQVAP |
| | | | | VLYQPHLEALDRRLRVVLKAVRDCVERN |
| | | | | GLHSVVDDDLDTEHRAASARDYKDDDD |
| | | | | K |
| 137 | ScMNN1 | Fam20c | ScMNN1 | MLALRRFILNQRSLRSCTIPILVGALIILV |
| | (M1-Q93) | (D93- | (M1-Q93)_ | LFQLVTHRNDALIRSSNVNSTNKKTLKDA |
| | | R584) | Fam20c | DPKVLIEAFGSPEVDPVDTIPVSPLELVPF |
| | | | (D93- | YDQDFSSDPSSNLSSHSLEKLPPAAEPAER |
| | | | R584)_FLAG | ALRGRDPGALRPHDPAHRPLLRDPGPRR |
| | | | | SESPPGPGGDASLLARLFEHPLYRVAVPP |
| | | | | LTEEDVLFNVNSDTRLSPKAAENPDWPH |
| | | | | AGAEGAEFLSPGEAAVDSYPNWLKFHIGI |
| | | | | NRYELYSRHNPAIEALLHDLSSQRITSVA |
| | | | | MKSGGTQLKLIMTFQNYGQALFKPMKQ |
| | | | | TREQETPPDFFYFSDYERHNAEIAAFHLD |
| | | | | RILDFRRVPPVAGRMVNMTKEIRDVTRD |
| | | | | KKLWRTFFISPANNICFYGECSYYCSTEH |
| | | | | ALCGKPDQIEGSLAAFLPDLSLAKRKTW |
| | | | | RNPWRRSYHKRKKAEWEVDPDYCEEVK |
| | | | | QTPPYDSSHRILDVMDMTIFDFLMGNMD |
| | | | | RHHYETFEKFGNETFIIHLDNGRGFGKYS |
| | | | | HDELSILVPLQQCCRIRKSTYLRLQLLAK |
| | | | | EEYKLSLLMAESLRGDQVAPVLYQPHLE |
| | | | | ALDRRLRVVLKAVRDCVERNGLHSVVD |
| | | | | DDLDTEHRAASARDYKDDDDK |
| 138 | ScMNN1 | Fam20c | ScMNN1( | MLALRRFILNQRSLRSCTIPILVGALIILV |
| | (M1-G153) | (D93- | M1- | LFQLVTHRNDALIRSSNVNSTNKKTLKDA |
| | | R584) | G153)_Fa | DPKVLIEAFGSPEVDPVDTIPVSPLELVPF |
| | | | m20c(D93- | YDQSIDTKRSSSWLINKKGYYKHFNELSL |
| | | | R584)_FLAG | TDRCKFYFRTLYTLDDEWTNSVKKLEYS |
| | | | | INDNEGDFSSDPSSNLSSHSLEKLPPAAEP |
| | | | | AERALRGRDPGALRPHDPAHRPLLRDPG |
| | | | | PRRSESPPGPGGDASLLARLFEHPLYRVA |
| | | | | VPPLTEEDVLFNVNSDTRLSPKAAENPD |
| | | | | WPHAGAEGAEFLSPGEAAVDSYPNWLKF |
| | | | | HIGINRYELYSRHNPAIEALLHDLSSQRIT |
| | | | | SVAMKSGGTQLKLIMTFQNYGQALFKP |
| | | | | MKQTREQETPPDFFYFSDYERHNAEIAAF |
| | | | | HLDRILDFRRVPPVAGRMVNMTKEIRDV |
| | | | | TRDKKLWRTFFISPANNICFYGECSYYCS |
| | | | | TEHALCGKPDQIEGSLAAFLPDLSLAKRK |
| | | | | TWRNPWRRSYHKRKKAEWEVDPDYCEE |
| | | | | VKQTPPYDSSHRILDVMDMTIFDFLMGN |
| | | | | MDRHHYETFEKFGNETFIIHLDNGRGFG |
| | | | | KYSHDELSILVPLQQCCRIRKSTYLRLQL |
| | | | | LAKEEYKLSLLMAESLRGDQVAPVLYQP |
| | | | | HLEALDRRLRVVLKAVRDCVERNGLHSV |
| | | | | VDDDLDTEHRAASARDYKDDDDK |
| 139 | ScMNN6 | Fam20c | ScMNN6( | MHVLLSKKIARFLLISFVFVLALMVTINH |
| | (M1-P30) | (D93- | M1- | PDFSSDPSSNLSSHSLEKLPPAAEPAERAL |
| | | R584) | P30)_Fam | RGRDPGALRPHDPAHRPLLRDPGPRRSE |
| | | | 20c(D93- | SPPGPGGDASLLARLFEHPLYRVAVPPLT |
| | | | R584)_FLAG | EEDVLFNVNSDTRLSPKAAENPDWPHAG |
| | | | | AEGAEFLSPGEAAVDSYPNWLKFHIGINR |
| | | | | YELYSRHNPAIEALLHDLSSQRITSVAMK |
| | | | | SGGTQLKLIMTFQNYGQALFKPMKQTR |
| | | | | EQETPPDFFYFSDYERHNAEIAAFHLDRI |
| | | | | LDFRRVPPVAGRMVNMTKEIRDVTRDK |
| | | | | KLWRTFFISPANNICFYGECSYYCSTEHA |
| | | | | LCGKPDQIEGSLAAFLPDLSLAKRKTWR |
| | | | | NPWRRSYHKRKKAEWEVDPDYCEEVKQ |
| | | | | TPPYDSSHRILDVMDMTIFDFLMGNMDR |
| | | | | HHYETFEKFGNETFIIHLDNGRGFGKYSH |
| | | | | DELSILVPLQQCCRIRKSTYLRLQLLAKE |
| | | | | EYKLSLLMAESLRGDQVAPVLYQPHLEA |
| | | | | LDRRLRVVLKAVRDCVERNGLHSVVDD |
| | | | | DLDTEHRAASARDYKDDDDK |
| 140 | ScMNN6 | Fam20c | ScMNN6( | MHVLLSKKIARFLLISFVFVLALMVTINH |
| | (M1-V85) | (D93- | M1- | PKTKQMSEQYVTPYLPKSLQPIAKISAEE |
| | | R584) | V85)_Fam | QRRIQSEQEEAELKQSLEGEAIRNATVDF |
| | | | 20c(D93- | SSDPSSNLSSHSLEKLPPAAEPAERALRGR |
| | | | R584)_FLAG | DPGALRPHDPAHRPLLRDPGPRRSESPPG |
| | | | | PGGDASLLARLFEHPLYRVAVPPLTEEDV |
| | | | | LFNVNSDTRLSPKAAENPDWPHAGAEGA |
| | | | | EFLSPGEAAVDSYPNWLKFHIGINRYELY |
| | | | | SRHNPAIEALLHDLSSQRITSVAMKSGGT |
| | | | | QLKLIMTFQNYGQALFKPMKQTREQET |
| | | | | PPDFFYFSDYERHNAEIAAFHLDRILDFR |
| | | | | RVPPVAGRMVNMTKEIRDVTRDKKLWR |
| | | | | TFFISPANNICFYGECSYYCSTEHALCGKP |
| | | | | DQIEGSLAAFLPDLSLAKRKTWRNPWRR |
| | | | | SYHKRKKAEWEVDPDYCEEVKQTPPYDS |
| | | | | SHRILDVMDMTIFDFLMGNMDRHHYETF |
| | | | | EKFGNETFIIHLDNGRGFGKYSHDELSIL |
| | | | | VPLQQCCRIRKSTYLRLQLLAKEEYKLS |
| | | | | LLMAESLRGDQVAPVLYQPHLEALDRRL |
| | | | | RVVLKAVRDCVERNGLHSVVDDDLDTE |
| | | | | HRAASARDYKDDDDK |
| 141 | ScMNN6 | Fam20c | ScMNN6( | MHVLLSKKIARFLLISFVFVLALMVTINH |
| | (M1-E160) | (D93- | M1- | PKTKQMSEQYVTPYLPKSLQPIAKISAEE |
| | | R584) | E160)_Fa | QRRIQSEQEEAELKQSLEGEAIRNATVNA |
| | | | m20c(D93- | IKEKIKSYGGNETTLGFMVPSYINHRGSP |
| | | | R584)_FLAG | PKACFVSLITERDSMTQILQSIDEVQVKF |
| | | | | NKNFAYPWVFISQGEDFSSDPSSNLSSHSL |
| | | | | EKLPPAAEPAERALRGRDPGALRPHDPA |
| | | | | HRPLLRDPGPRRSESPPGPGGDASLLARL |
| | | | | FEHPLYRVAVPPLTEEDVLFNVNSDTRLS |
| | | | | PKAAENPDWPHAGAEGAEFLSPGEAAVD |
| | | | | SYPNWLKFHIGINRYELYSRHNPAIEALL |
| | | | | HDLSSQRITSVAMKSGGTQLKLIMTFQN |
| | | | | YGQALFKPMKQTREQETPPDFFYFSDYE |
| | | | | RHNAEIAAFHLDRILDFRRVPPVAGRMV |
| | | | | NMTKEIRDVTRDKKLWRTFFISPANNICF |
| | | | | YGECSYYCSTEHALCGKPDQIEGSLAAFL |
| | | | | PDLSLAKRKTWRNPWRRSYHKRKKAEW |
| | | | | EVDPDYCEEVKQTPPYDSSHRILDVMDM |
| | | | | TIFDFLMGNMDRHHYETFEKFGNETFIH |
| | | | | LDNGRGFGKYSHDELSILVPLQQCCRIRK |
| | | | | STYLRLQLLAKEEYKLSLLMAESLRGDQ |
| | | | | VAPVLYQPHLEALDRRLRVVLKAVRDCV |
| | | | | ERNGLHSVVDDDLDTEHRAASARDYKDD |
| | | | | DDK |
| 142 | N/A | Fam20c | Fam20c | RLERRGARPSGEPGCSCAQPAAEVAAPG |
| | | (R32- | (R32- | WAQVRGRPGEPPAASSAAGDAGWPNKH |
| | | R584) | R584) | TLRILQDFSSDPSSNLSSHSLEKLPPAAEP |
| | | | | AERALRGRDPGALRPHDPAHRPLLRDPG |
| | | | | PRRSESPPGPGGDASLLARLFEHPLYRVA |
| | | | | VPPLTEEDVLFNVNSDTRLSPKAAENPD |
| | | | | WPHAGAEGAEFLSPGEAAVDSYPNWLKF |
| | | | | HIGINRYELYSRHNPAIEALLHDLSSQRIT |
| | | | | SVAMKSGGTQLKLIMTFQNYGQALFKP |
| | | | | MKQTREQETPPDFFYFSDYERHNAEIAAF |
| | | | | HLDRILDFRRVPPVAGRMVNMTKEIRDV |
| | | | | TRDKKLWRTFFISPANNICFYGECSYYCS |
| | | | | TEHALCGKPDQIEGSLAAFLPDLSLAKRK |
| | | | | TWRNPWRRSYHKRKKAEWEVDPDYCEE |
| | | | | VKQTPPYDSSHRILDVMDMTIFDFLMGN |
| | | | | MDRHHYETFEKFGNETFIIHLDNGRGFG |
| | | | | KYSHDELSILVPLQQCCRIRKSTYLRLQL |
| | | | | LAKEEYKLSLLMAESLRGDQVAPVLYQP |
| | | | | HLEALDRRLRVVLKAVRDCVERNGLHSV |
| | | | | VDDDLDTEHRAASAR |
| 143 | N/A | Fam20c | Fam20c | RGRPGEPPAASSAAGDAGWPNKHTLRIL |
| | | (R64- | (R64- | QDFSSDPSSNLSSHSLEKLPPAAEPAERAL |
| | | R584) | R584) | RGRDPGALRPHDPAHRPLLRDPGPRRSE |
| | | | | SPPGPGGDASLLARLFEHPLYRVAVPPLT |
| | | | | EEDVLFNVNSDTRLSPKAAENPDWPHAG |
| | | | | AEGAEFLSPGEAAVDSYPNWLKFHIGINR |
| | | | | YELYSRHNPAIEALLHDLSSQRITSVAMK |
| | | | | SGGTQLKLIMTFQNYGQALFKPMKQTR |
| | | | | EQETPPDFFYFSDYERHNAEIAAFHLDRI |
| | | | | LDFRRVPPVAGRMVNMTKEIRDVTRDK |
| | | | | KLWRTFFISPANNICFYGECSYYCSTEHA |
| | | | | LCGKPDQIEGSLAAFLPDLSLAKRKTWR |
| | | | | NPWRRSYHKRKKAEWEVDPDYCEEVKQ |
| | | | | TPPYDSSHRILDVMDMTIFDFLMGNMDR |
| | | | | HHYETFEKFGNETFIIHLDNGRGFGKYSH |
| | | | | DELSILVPLQQCCRIRKSTYLRLQLLAKE |
| | | | | EYKLSLLMAESLRGDQVAPVLYQPHLEA |
| | | | | LDRRLRVVLKAVRDCVERNGLHSVVDD |
| | | | | DLDTEHRAASAR |
| 144 | N/A | Fam20c | Fam20c | DFSSDPSSNLSSHSLEKLPPAAEPAERALR |
| | | (D93- | (D93- | GRDPGALRPHDPAHRPLLRDPGPRRSESP |
| | | R584) | R584) | PGPGGDASLLARLFEHPLYRVAVPPLTEE |
| | | | | DVLFNVNSDTRLSPKAAENPDWPHAGAE |
| | | | | GAEFLSPGEAAVDSYPNWLKFHIGINRYE |
| | | | | LYSRHINPAIEALLHDLSSQRITSVAMKSG |
| | | | | GTQLKLIMTFQNYGQALFKPMKQTREQ |
| | | | | ETPPDFFYFSDYERHNAEIAAFHLDRILDF |
| | | | | RRVPPVAGRMVNMTKEIRDVTRDKKLW |
| | | | | RTFFISPANNICFYGECSYYCSTEHALCG |
| | | | | KPDQIEGSLAAFLPDLSLAKRKTWRNPW |
| | | | | RRSYHKRKKAEWEVDPDYCEEVKQTPP |
| | | | | YDSSHRILDVMDMTIFDFLMGNMDRHH |
| | | | | YETFEKFGNETFIIHLDNGRGFGKYSHDE |
| | | | | LSILVPLQQCCRIRKSTYLRLQLLAKEEY |
| | | | | KLSLLMAESLRGDQVAPVLYQPHLEALD |
| | | | | RRLRVVLKAVRDCVERNGLHSVVDDDL |
| | | | | DTEHRAASAR |
| 154 | Native | Fam20c | Fam20c(M | MKMMLVRRFRVLILMVFLVACALHIAL |
| | Fam20c | (M1-R584) | 1-R584)_ | DLLPRLERRGARPSGEPGCSCAQPAAEV |
| | derived | | MYC | AAPGWAQVRGRPGEPPAASSAAGDAGW |
| | | | | PNKHTLRILQDFSSDPSSNLSSHSLEKLPP |
| | | | | AAEPAERALRGRDPGALRPHDPAHRPLL |
| | | | | RDPGPRRSESPPGPGGDASLLARLFEHPL |
| | | | | YRVAVPPLTEEDVLFNVNSDTRLSPKAAE |
| | | | | NPDWPHAGAEGAEFLSPGEAAVDSYPNW |
| | | | | LKFHIGINRYELYSRHNPAIEALLHDLSSQ |
| | | | | RITSVAMKSGGTQLKLIMTFQNYGQALF |
| | | | | KPMKQTREQETPPDFFYFSDYERHNAEI |
| | | | | AAFHLDRILDFRRVPPVAGRMVNMTKEI |
| | | | | RDVTRDKKLWRTFFISPANNICFYGECSY |
| | | | | YCSTEHALCGKPDQIEGSLAAFLPDLSLA |
| | | | | KRKTWRNPWRRSYHKRKKAEWEVDPD |
| | | | | YCEEVKQTPPYDSSHRILDVMDMTIFDFL |
| | | | | MGNMDRHHYETFEKFGNETFHIHLDNGR |
| | | | | GFGKYSHDELSILVPLQQCCRIRKSTYLR |
| | | | | LQLLAKEEYKLSLLMAESLRGDQVAPVL |
| | | | | YQPHLEALDRRLRVVLKAVRDCVERNG |
| | | | | LHSVVDDDLDTEHRAASAREQKLISEEDL |
| 313 | Native | Fam20c | Fam20c(M | MKMMLVRRFRVLILMVFLVACALHIAL |
| | Fam20c | (M1- | 1-R584) | DLLPRLERRGARPSGEPGCSCAQPAAEV |
| | derived | R584) | | AAPGWAQVRGRPGEPPAASSAAGDAGW |
| | | | | PNKHTLRILQDFSSDPSSNLSSHSLEKLPP |
| | | | | AAEPAERALRGRDPGALRPHDPAHRPLL |
| | | | | RDPGPRRSESPPGPGGDASLLARLFEHPL |
| | | | | YRVAVPPLTEEDVLFNVNSDTRLSPKAAE |
| | | | | NPDWPHAGAEGAEFLSPGEAAVDSYPNW |
| | | | | LKFHIGINRYELYSRHNPAIEALLHDLSSQ |
| | | | | RITSVAMKSGGTQLKLIMTFQNYGQALF |
| | | | | KPMKQTREQETPPDFFYFSDYERHNAEI |
| | | | | AAFHLDRILDERRVPPVAGRMVNMTKEI |
| | | | | RDVTRDKKLWRTFFISPANNICFYGECSY |
| | | | | YCSTEHALCGKPDQIEGSLAAFLPDLSLA |
| | | | | KRKTWRNPWRRSYHKRKKAEWEVDPD |
| | | | | YCEEVKQTPPYDSSHRILDVMDMTIFDFL |
| | | | | MGNMDRHHYETFEKFGNETFIIHLDNGR |
| | | | | GFGKYSHDELSILVPLQQCCRIRKSTYLR |
| | | | | LQLLAKEEYKLSLLMAESLRGDQVAPVL |
| | | | | YQPHLEALDRRLRVVLKAVRDCVERNG |
| | | | | LHSVVDDDLDTEHRAASAR |
| 314 | ScKRE2 | Fam20c | ScKRE(M | MALFLSKRLLRFTVIAGAVIVLLLTLNSN |
| | (M1-158) | (R32- | 1-158)_ | SRTQQYIPSSISAAFDFTSGSISPEQQVIRL |
| | | R584) | Fam20c | ERRGARPSGEPGCSCAQPAAEVAAPGWA |
| | | | (R32- | QVRGRPGEPPAASSAAGDAGWPNKHTL |
| | | | R584) | RILQDESSDPSSNLSSHSLEKLPPAAEPAE |
| | | | | RALRGRDPGALRPHDPAHRPLLRDPGPR |
| | | | | RSESPPGPGGDASLLARLFEHPLYRVAVP |
| | | | | PLTEEDVLENVNSDTRLSPKAAENPDWP |
| | | | | HAGAEGAEFLSPGEAAVDSYPNWLKFHI |
| | | | | GINRYELYSRHNPAIEALLHDLSSQRITSV |
| | | | | AMKSGGTQLKLIMTFQNYGQALFKPMK |
| | | | | QTREQETPPDFFYFSDYERHNAEIAAFHL |
| | | | | DRILDFRRVPPVAGRMVNMTKEIRDVTR |
| | | | | DKKLWRTFFISPANNICFYGECSYYCSTE |
| | | | | HALCGKPDQIEGSLAAFLPDLSLAKRKT |
| | | | | WRNPWRRSYHKRKKAEWEVDPDYCEE |
| | | | | VKQTPPYDSSHRILDVMDMTIFDELMGN |
| | | | | MDRHHYETFEKFGNETFIIHLDNGRGFG |
| | | | | KYSHDELSILVPLQQCCRIRKSTYLRLQL |
| | | | | LAKEEYKLSLLMAESLRGDQVAPVLYQP |
| | | | | HLEALDRRLRVVLKAVRDCVERNGLHSV |
| | | | | VDDDLDTEHRAASAR |
| 315 | ScKRE2 | Fam20c | ScKRE2 | MALFLSKRLLRFTVIAGAVIVLLLTLNSN |
| | (M1-S80) | (R32- | (M1-S80)_ | SRTQQYIPSSISAAFDFTSGSISPEQQVISE |
| | ScKRE2 | R584) | Fam20c | ENDAKKLEQSALNSEASEDSRLERRGAR |
| | | | (R32- | PSGEPGCSCAQPAAEVAAPGWAQVRGRP |
| | | | R584) | GEPPAASSAAGDAGWPNKHTLRILQDFSS |
| | | | | DPSSNLSSHSLEKLPPAAEPAERALRGRD |
| | | | | PGALRPHDPAHRPLLRDPGPRRSESPPGP |
| | | | | GGDASLLARLFEHPLYRVAVPPLTEEDV |
| | | | | LFNVNSDTRLSPKAAENPDWPHAGAEGA |
| | | | | EFLSPGEAAVDSYPNWLKFHIGINRYELY |
| | | | | SRHNPAIEALLHDLSSQRITSVAMKSGGT |
| | | | | QLKLIMTFQNYGQALFKPMKQTREQET |
| | | | | PPDFFYFSDYERHNAEIAAFHLDRILDFR |
| | | | | RVPPVAGRMVNMTKEIRDVTRDKKLWR |
| | | | | TFFISPANNICFYGECSYYCSTEHALCGKP |
| | | | | DQIEGSLAAFLPDLSLAKRKTWRNPWRR |
| | | | | SYHKRKKAEWEVDPDYCEEVKQTPPYDS |
| | | | | SHRILDVMDMTIFDFLMGNMDRHHYETF |
| | | | | EKFGNETFIIHLDNGRGFGKYSHDELSIL |
| | | | | VPLQQCCRIRKSTYLRLQLLAKEEYKLS |
| | | | | LLMAESLRGDQVAPVLYQPHLEALDRRL |
| | | | | RVVLKAVRDCVERNGLHSVVDDDLDTE |
| | | | | HRAASAR |
| 316 | (M1-D102) | Fam20c | ScKRE2 | MALFLSKRLLRFTVIAGAVIVLLLTLNSN |
| | | (R32- | (M1-D102)_ | SRTQQYIPSSISAAFDFTSGSISPEQQVISE |
| | | R584) | Fam20c | ENDAKKLEQSALNSEASEDSEAMDEESK |
| | | | (R32- | ALKAAAEKADAPIDRLERRGARPSGEPG |
| | | | R584) | CSCAQPAAEVAAPGWAQVRGRPGEPPA |
| | | | | ASSAAGDAGWPNKHTLRILQDFSSDPSSN |
| | | | | LSSHSLEKLPPAAEPAERALRGRDPGALR |
| | | | | PHDPAHRPLLRDPGPRRSESPPGPGGDAS |
| | | | | LLARLFEHPLYRVAVPPLTEEDVLENVNS |
| | | | | DTRLSPKAAENPDWPHAGAEGAEFLSPG |
| | | | | EAAVDSYPNWLKFHIGINRYELYSRHNPA |
| | | | | IEALLHDLSSQRITSVAMKSGGTQLKLIM |
| | | | | TFQNYGQALFKPMKQTREQETPPDFFYF |
| | | | | SDYERHNAEIAAFHLDRILDFRRVPPVAG |
| | | | | RMVNMTKEIRDVTRDKKLWRTFFISPAN |
| | | | | NICFYGECSYYCSTEHALCGKPDQIEGSL |
| | | | | AAFLPDLSLAKRKTWRNPWRRSYHKRK |
| | | | | KAEWEVDPDYCEEVKQTPPYDSSHRILD |
| | | | | VMDMTIFDFLMGNMDRHHYETFEKFGN |
| | | | | ETFIHLDNGRGFGKYSHDELSILVPLQQ |
| | | | | CCRIRKSTYLRLQLLAKEEYKLSLLMAE |
| | | | | SLRGDQVAPVLYQPHLEALDRRLRVVLK |
| | | | | AVRDCVERNGLHSVVDDDLDTEHRAASA |
| | | | | R |
| 317 | PpKRE2 | Fam20c | PpKRE2( | MVHIGFRSLKAVFILALSSLILYGIVTTFD |
| | (M1-G31) | (R32- | M1-G31)_ | GRLERRGARPSGEPGCSCAQPAAEVAAP |
| | | R584) | Fam20c | GWAQVRGRPGEPPAASSAAGDAGWPNK |
| | | | (R32- | HTLRILQDFSSDPSSNLSSHSLEKLPPAAE |
| | | | R584) | PAERALRGRDPGALRPHDPAHRPLLRDP |
| | | | | GPRRSESPPGPGGDASLLARLFEHPLYRV |
| | | | | AVPPLTEEDVLFNVNSDTRLSPKAAENPD |
| | | | | WPHAGAEGAEFLSPGEAAVDSYPNWLKF |
| | | | | HIGINRYELYSRUNPAIEALLHDLSSQRIT |
| | | | | SVAMKSGGTQLKLIMTFQNYGQALFKP |
| | | | | MKQTREQETPPDFFYFSDYERHNAEIAAF |
| | | | | HLDRILDFRRVPPVAGRMVNMTKEIRDV |
| | | | | TRDKKLWRTFFISPANNICFYGECSYYCS |
| | | | | TEHALCGKPDQIEGSLAAFLPDLSLAKRK |
| | | | | TWRNPWRRSYHKRKKAEWEVDPDYCEE |
| | | | | VKQTPPYDSSHRILDVMDMTIFDFLMGN |
| | | | | MDRHHYETFEKEGNETFIIHLDNGRGFG |
| | | | | KYSHDELSILVPLQQCCRIRKSTYLRLQL |
| | | | | LAKEEYKLSLLMAESLRGDQVAPVLYQP |
| | | | | HLEALDRRLRVVLKAVRDCVERNGLHSV |
| | | | | VDDDLDTEHRAASAR |
| 318 | PpKRE2 | Fam20c | PpKRE2( | MVHIGFRSLKAVFILALSSLILYGIVTTFD |
| | (M1-D84) | (R32- | M1- | GSRASRYQPPYVNHSQDPLYHSGNSYNR |
| | | R584) | D84)_Fam | ENATFVTLCRNEDLYSHIQSIKKVEDRLER |
| | | | 20c(R32- | RGARPSGEPGCSCAQPAAEVAAPGWAQ |
| | | | R584) | VRGRPGEPPAASSAAGDAGWPNKHTLRI |
| | | | | LQDFSSDPSSNLSSHSLEKLPPAAEPAERA |
| | | | | LRGRDPGALRPHDPAHRPLLRDPGPRRS |
| | | | | ESPPGPGGDASLLARLFEHPLYRVAVPPL |
| | | | | TEEDVLFNVNSDTRLSPKAAENPDWPHA |
| | | | | GAEGAEFLSPGEAAVDSYPNWLKFHIGIN |
| | | | | RYELYSRHNPAIEALLHDLSSQRITSVAM |
| | | | | KSGGTQLKLIMTFQNYGQALFKPMKQT |
| | | | | REQETPPDFFYFSDYERHNAEIAAFHLDR |
| | | | | ILDFRRVPPVAGRMVNMTKEIRDVTRDK |
| | | | | KLWRTFFISPANNICFYGECSYYCSTEHA |
| | | | | LCGKPDQIEGSLAAFLPDLSLAKRKTWR |
| | | | | NPWRRSYHKRKKAEWEVDPDYCEEVKQ |
| | | | | TPPYDSSHRILDVMDMTIFDFLMGNMDR |
| | | | | HHYETFEKFGNETFIHLDNGRGFGKYSH |
| | | | | DELSILVPLQQCCRIRKSTYLRLQLLAKE |
| | | | | EYKLSLLMAESLRGDQVAPVLYQPHLEA |
| | | | | LDRRLRVVLKAVRDCVERNGLHSVVDD |
| | | | | DLDTEHRAASAR |
| 319 | PpKRE2 | Fam20c | PpKRE2( | MVHIGERSLKAVFILALSSLILYGIVTTED |
| | (M1-H150) | (R32- | M1- | GSRASRYQPPYVNHSQDPLYHSGNSYNR |
| | | R584) | H150)_Fa | ENATFVTLCRNEDLYSHIQSIKKVEDRENN |
| | | | m20c(R32- | KFAYDWVFLNEVPFTDEFKERTSVLISGQ |
| | | | R584) | AKYGLIPKEHWSYPDYIDQERAAESRRQ |
| | | | | LEDQHRLERRGARPSGEPGCSCAQPAAE |
| | | | | VAAPGWAQVRGRPGEPPAASSAAGDAG |
| | | | | WPNKHTLRILQDFSSDPSSNLSSHSLEKLP |
| | | | | PAAEPAERALRGRDPGALRPHDPAHRPL |
| | | | | LRDPGPRRSESPPGPGGDASLLARLFEHP |
| | | | | LYRVAVPPLTEEDVLFNVNSDTRLSPKAA |
| | | | | ENPDWPHAGAEGAEFLSPGEAAVDSYPN |
| | | | | WLKFHIGINRYELYSRHNPAIEALLEDLS |
| | | | | SQRITSVAMKSGGTQLKLIMTFQNYGQA |
| | | | | LFKPMKQTREQETPPDFFYFSDYERHNA |
| | | | | EIAAFHLDRILDFRRVPPVAGRMVNMTK |
| | | | | EIRDVTRDKKLWRTFFISPANNICFYGEC |
| | | | | SYYCSTEHALCGKPDQIEGSLAAFLPDLS |
| | | | | LAKRKTWRNPWRRSYHKRKKAEWEVD |
| | | | | PDYCEEVKQTPPYDSSHRILDVMDMTIFD |
| | | | | FLMGNMDRHHYETFEKFGNETFIIHLDN |
| | | | | GRGFGKYSHDELSILVPLQQCCRIRKSTY |
| | | | | LRLQLLAKEEYKLSLLMAESLRGDQVAP |
| | | | | VLYQPHLEALDRRLRVVLKAVRDCVERN |
| | | | | GLHSVVDDDLDTEHRAASAR |
| 320 | ScMNN2 | Fam20c | ScMNN2( | MLLTKRFSKLFKLTFIVLILCGLFVITNK |
| | (M1-S36) | (R32- | M1- | YMDENTSRLERRGARPSGEPGCSCAQPA |
| | | R584) | S36)_Fam | AEVAAPGWAQVRGRPGEPPAASSAAGDA |
| | | | 20c(R32- | GWPNKHTLRILQDFSSDPSSNLSSHSLEK |
| | | | R584) | LPPAAEPAERALRGRDPGALRPHDPAHR |
| | | | | PLLRDPGPRRSESPPGPGGDASLLARLFE |
| | | | | HPLYRVAVPPLTEEDVLFNVNSDTRLSPK |
| | | | | AAENPDWPHAGAEGAEFLSPGEAAVDSY |
| | | | | PNWLKFHIGINRYELYSRHNPAIEALLHD |
| | | | | LSSQRITSVAMKSGGTQLKLIMTFQNYG |
| | | | | QALFKPMKQTREQETPPDFFYFSDYERH |
| | | | | NAEIAAFHLDRILDFRRVPPVAGRMVNM |
| | | | | TKEIRDVTRDKKLWRTFFISPANNICFYG |
| | | | | ECSYYCSTEHALCGKPDQIEGSLAAFLPD |
| | | | | LSLAKRKTWRNPWRRSYHKRKKAEWE |
| | | | | VDPDYCEEVKQTPPYDSSHRILDVMDMT |
| | | | | IFDFLMGNMDRHHYETFEKFGNETFIIHL |
| | | | | DNGRGFGKYSHDELSILVPLQQCCRIRKS |
| | | | | TYLRLQLLAKEEYKLSLLMAESLRGDQV |
| | | | | APVLYQPHLEALDRRLRVVLKAVRDCVE |
| | | | | RNGLHSVVDDDLDTEHRAASAR |
| 321 | ScMNN2 | Fam20c | ScMNN2( | MLLTKRFSKLFKLTFIVLILCGLFVITNK |
| | (M1-P97) | (R32- | M1- | YMDENTSVKEYKEYLDRYVQSYSNKYSS |
| | | R584) | P97)_Fam | SSDAASADDSTPLRDNDEAGNEKLKSFYN |
| | | | 20c(R32- | NVFNFLMVDSPRLERRGARPSGEPGCSC |
| | | | R584) | AQPAAEVAAPGWAQVRGRPGEPPAASSA |
| | | | | AGDAGWPNKHTLRILQDFSSDPSSNLSSH |
| | | | | SLEKLPPAAEPAERALRGRDPGALRPHD |
| | | | | PAHRPLLRDPGPRRSESPPGPGGDASLLA |
| | | | | RLFEHPLYRVAVPPLTEEDVLFNVNSDTR |
| | | | | LSPKAAENPDWPHAGAEGAEFLSPGEAA |
| | | | | VDSYPNWLKFHIGINRYELYSRHNPAIEA |
| | | | | LLHDLSSQRITSVAMKSGGTQLKLIMTF |
| | | | | QNYGQALFKPMKQTREQETPPDFFYFSD |
| | | | | YERHNAEIAAFHLDRILDFRRVPPVAGR |
| | | | | MVNMTKEIRDVTRDKKLWRTFFISPANN |
| | | | | ICFYGECSYYCSTEHALCGKPDQIEGSLA |
| | | | | AFLPDLSLAKRKTWRNPWRRSYHKRKK |
| | | | | AEWEVDPDYCEEVKQTPPYDSSHRILDV |
| | | | | MDMTIFDFLMGNMDRHHYETFEKFGNE |
| | | | | TFIIHLDNGRGFGKYSHDELSILVPLQQC |
| | | | | CRIRKSTYLRLQLLAKEEYKLSLLMAESL |
| | | | | RGDQVAPVLYQPHLEALDRRLRVVLKA |
| | | | | VRDCVERNGLHSVVDDDLDTEHRAASAR |
| 322 | ScMNN2 | Fam20c | ScMNN2( | MLLTKRFSKLFKLTFIVLILCGLFVITNK |
| | (M1-S150) | (R32- | M1-S150)_ | YMDENTSVKEYKEYLDRYVQSYSNKYSS |
| | | R584) | Fam20c | SSDAASADDSTPLRDNDEAGNEKLKSFYN |
| | | | (R32- | NVFNFLMVDSPKGSTAKQYNEACLLKGD |
| | | | R584) | IGDRPDHYKDLYKLSAKELSKCLELSPDE |
| | | | | VASLTKSRLERRGARPSGEPGCSCAQPA |
| | | | | AEVAAPGWAQVRGRPGEPPAASSAAGDA |
| | | | | GWPNKHTLRILQDFSSDPSSNLSSHSLEK |
| | | | | LPPAAEPAERALRGRDPGALRPHDPAHR |
| | | | | PLLRDPGPRRSESPPGPGGDASLLARLFE |
| | | | | HPLYRVAVPPLTEEDVLFNVNSDTRLSPK |
| | | | | AAENPDWPHAGAEGAEFLSPGEAAVDSY |
| | | | | PNWLKFHIGINRYELYSRANPAIEALLHD |
| | | | | LSSQRITSVAMKSGGTQLKLIMTFQNYG |
| | | | | QALFKPMKQTREQETPPDFFYFSDYERH |
| | | | | NAEIAAFHLDRILDFRRVPPVAGRMVNM |
| | | | | TKEIRDVTRDKKLWRTFFISPANNICFYG |
| | | | | ECSYYCSTEHALCGKPDQIEGSLAAFLPD |
| | | | | LSLAKRKTWRNPWRRSYHKRKKAEWE |
| | | | | VDPDYCEEVKQTPPYDSSHRILDVMDMT |
| | | | | IFDFLMGNMDRHHYETFEKFGNETFIIHL |
| | | | | DNGRGFGKYSHDELSILVPLQQCCRIRKS |
| | | | | TYLRLQLLAKEEYKLSLLMAESLRGDQV |
| | | | | APVLYQPHLEALDRRLRVVLKAVRDCVE |
| | | | | RNGLHSVVDDDLDTEHRAASAR |
| 323 | ScMNN1 | Fam20c | ScMNN1 | MLALRRFILNQRSLRSCTIPILVGALIILV |
| | (M1-A42) | (R32- | (M1- | LFQLVTHRNDARLERRGARPSGEPGCSC |
| | | R584) | A42)_Fam | AQPAAEVAAPGWAQVRGRPGEPPAASSA |
| | | | 20c(R32- | AGDAGWPNKHTLRILQDFSSDPSSNLSSH |
| | | | R584) | SLEKLPPAAEPAERALRGRDPGALRPHD |
| | | | | PAHRPLLRDPGPRRSESPPGPGGDASLLA |
| | | | | RLFEHPLYRVAVPPLTEEDVLFNVNSDTR |
| | | | | LSPKAAENPDWPHAGAEGAEFLSPGEAA |
| | | | | VDSYPNWLKFHIGINRYELYSRHNPAIEA |
| | | | | LLHDLSSQRITSVAMKSGGTQLKLIMTF |
| | | | | QNYGQALFKPMKQTREQETPPDFFYFSD |
| | | | | YERHNAEIAAFHLDRILDFRRVPPVAGR |
| | | | | MVNMTKEIRDVTRDKKLWRTFFISPANN |
| | | | | ICFYGECSYYCSTEHALCGKPDQIEGSLA |
| | | | | AFLPDLSLAKRKTWRNPWRRSYHKRKK |
| | | | | AEWEVDPDYCEEVKQTPPYDSSHRILDV |
| | | | | MDMTIFDFLMGNMDRHHYETFEKFGNE |
| | | | | TFIIHLDNGRGFGKYSHDELSILVPLQQC |
| | | | | CRIRKSTYLRLQLLAKEEYKLSLLMAESL |
| | | | | RGDQVAPVLYQPHLEALDRRLRVVLKA |
| | | | | VRDCVERNGLHSVVDDDLDTEHRAASAR |
| 324 | ScMNN1 | Fam20c | ScMNN1 | MLALRRFILNQRSLRSCTIPILVGALIUILV |
| | (M1-Q93) | (R32- | (M1-Q93)_ | LFQLVTHRNDALIRSSNVNSTNKKTLKDA |
| | | R584) | Fam20c | DPKVLIEAFGSPEVDPVDTIPVSPLELVPF |
| | | | (R32- | YDQRLERRGARPSGEPGCSCAQPAAEVA |
| | | | R584) | APGWAQVRGRPGEPPAASSAAGDAGWP |
| | | | | NKHTLRILQDFSSDPSSNLSSHSLEKLPPA |
| | | | | AEPAERALRGRDPGALRPHDPAHRPLLR |
| | | | | DPGPRRSESPPGPGGDASLLARLFEHPLY |
| | | | | RVAVPPLTEEDVLFNVNSDTRLSPKAAEN |
| | | | | PDWPHAGAEGAEFLSPGEAAVDSYPNWL |
| | | | | KFHIGINRYELYSRHNPAIEALLHDLSSQ |
| | | | | RITSVAMKSGGTQLKLIMTFQNYGQALF |
| | | | | KPMKQTREQETPPDFFYFSDYERHNAEI |
| | | | | AAFHLDRILDFRRVPPVAGRMVNMTKEI |
| | | | | RDVTRDKKLWRTFFISPANNICFYGECSY |
| | | | | YCSTEHALCGKPDQIEGSLAAFLPDLSLA |
| | | | | KRKTWRNPWRRSYHKRKKAEWEVDPD |
| | | | | YCEEVKQTPPYDSSHRILDVMDMTIFDFL |
| | | | | MGNMDRHHYETFEKFGNETFIIHLDNGR |
| | | | | GFGKYSHDELSILVPLQQCCRIRKSTYLR |
| | | | | LQLLAKEEYKLSLLMAESLRGDQVAPVL |
| | | | | YQPHLEALDRRLRVVLKAVRDCVERNG |
| | | | | LHSVVDDDLDTEHRAASAR |
| 325 | ScMNN1 | Fam20c | ScMNN1 | MLALRRFILNQRSLRSCTIPILVGALITILV |
| | (M1-G153) | (R32- | (M1-G153)_ | LFQLVTHRNDALIRSSNVNSTNKKTLKDA |
| | | R584) | Fam20c | DPKVLIEAFGSPEVDPVDTIPVSPLELVPF |
| | | | (R32- | YDQSIDTKRSSSWLINKKGYYKHFNELSL |
| | | | R584) | TDRCKFYFRTLYTLDDEWTNSVKKLEYS |
| | | | | INDNEGRLERRGARPSGEPGCSCAQPAA |
| | | | | EVAAPGWAQVRGRPGEPPAASSAAGDA |
| | | | | GWPNKHTLRILQDFSSDPSSNLSSHSLEK |
| | | | | LPPAAEPAERALRGRDPGALRPHDPAHR |
| | | | | PLLRDPGPRRSESPPGPGGDASLLARLFE |
| | | | | HPLYRVAVPPLTEEDVLFNVNSDTRLSPK |
| | | | | AAENPDWPHAGAEGAEFLSPGEAAVDSY |
| | | | | PNWLKFHIGINRYELYSRHNPAIEALLHD |
| | | | | LSSQRITSVAMKSGGTQLKLIMTFQNYG |
| | | | | QALFKPMKQTREQETPPDFFYFSDYERH |
| | | | | NAEIAAFHLDRILDFRRVPPVAGRMVNM |
| | | | | TKEIRDVTRDKKLWRTFFISPANNICFYG |
| | | | | ECSYYCSTEHALCGKPDQIEGSLAAFLPD |
| | | | | LSLAKRKTWRNPWRRSYHKRKKAEWE |
| | | | | VDPDYCEEVKQTPPYDSSHRILDVMDMT |
| | | | | IFDFLMGNMDRHHYETFEKFGNETFIHL |
| | | | | DNGRGFGKYSHDELSILVPLQQCCRIRKS |
| | | | | TYLRLQLLAKEEYKLSLLMAESLRGDQV |
| | | | | APVLYQPHLEALDRRLRVVLKAVRDCVE |
| | | | | RNGLHSVVDDDLDTEHRAASAR |
| 326 | ScMNN6 | Fam20c | ScMNN6 | MHVLLSKKIARFLLISFVFVLALMVTINH |
| | (M1-P30) | (R32- | (M1- | PRLERRGARPSGEPGCSCAQPAAEVAAP |
| | | R584) | P30)_Fam | GWAQVRGRPGEPPAASSAAGDAGWPNK |
| | | | 20c(R32- | HTLRILQDFSSDPSSNLSSHSLEKLPPAAE |
| | | | R584) | PAERALRGRDPGALRPHDPAHRPLLRDP |
| | | | | GPRRSESPPGPGGDASLLARLFEHPLYRV |
| | | | | AVPPLTEEDVLFNVNSDTRLSPKAAENPD |
| | | | | WPHAGAEGAEFLSPGEAAVDSYPNWLKF |
| | | | | HIGINRYELYSRHNPAIEALLHDLSSQRIT |
| | | | | SVAMKSGGTQLKLIMTFQNYGQALFKP |
| | | | | MKQTREQETPPDFFYFSDYERHNAEIAAF |
| | | | | HLDRILDFRRVPPVAGRMVNMTKEIRDV |
| | | | | TRDKKLWRTFFISPANNICFYGECSYYCS |
| | | | | TEHALCGKPDQIEGSLAAFLPDLSLAKRK |
| | | | | TWRNPWRRSYHKRKKAEWEVDPDYCEE |
| | | | | VKQTPPYDSSHRILDVMDMTIFDFLMGN |
| | | | | MDRHHYETFEKFGNETFIIHLDNGRGFG |
| | | | | KYSHDELSILVPLQQCCRIRKSTYLRLQL |
| | | | | LAKEEYKLSLLMAESLRGDQVAPVLYQP |
| | | | | HLEALDRRLRVVLKAVRDCVERNGLHSV |
| | | | | VDDDLDTEHRAASAR |
| 327 | ScMNN6 | Fam20c | ScMNN6 | MHVLLSKKIARFLLISFVFVLALMVTINH |
| | (M1-V85) | (R32- | (M1-V85)_ | PKTKQMSEQYVTPYLPKSLQPIAKISAEE |
| | | R584) | Fam20c | QRRIQSEQEEAELKQSLEGEAIRNATVRL |
| | | | (R32- | ERRGARPSGEPGCSCAQPAAEVAAPGWA |
| | | | R584) | QVRGRPGEPPAASSAAGDAGWPNKHTL |
| | | | | RILQDFSSDPSSNLSSHSLEKLPPAAEPAE |
| | | | | RALRGRDPGALRPHDPAHRPLLRDPGPR |
| | | | | RSESPPGPGGDASLLARLFEHPLYRVAVP |
| | | | | PLTEEDVLFNVNSDTRLSPKAAENPDWP |
| | | | | HAGAEGAEFLSPGEAAVDSYPNWLKFHI |
| | | | | GINRYELYSRHNPAIEALLHDLSSQRITSV |
| | | | | AMKSGGTQLKLIMTFQNYGQALFKPMK |
| | | | | QTREQETPPDFFYFSDYERHNAEIAAFHL |
| | | | | DRILDFRRVPPVAGRMVNMTKEIRDVTR |
| | | | | DKKLWRTFFISPANNICFYGECSYYCSTE |
| | | | | HALCGKPDQIEGSLAAFLPDLSLAKRKT |
| | | | | WRNPWRRSYHKRKKAEWEVDPDYCEE |
| | | | | VKQTPPYDSSHRILDVMDMTIFDFLMGN |
| | | | | MDRHHYETFEKFGNETFIIHLDNGRGFG |
| | | | | KYSHDELSILVPLQQCCRIRKSTYLRLQL |
| | | | | LAKEEYKLSLLMAESLRGDQVAPVLYQP |
| | | | | HLEALDRRLRVVLKAVRDCVERNGLHSV |
| | | | | VDDDLDTEHRAASAR |
| 328 | ScMNN6 | Fam20c | ScMNN6 | MHVLLSKKIARFLLISFVFVLALMVTINH |
| | (M1-E160) | (R32- | (M1-E160)_ | PKTKQMSEQYVTPYLPKSLQPIAKISAEE |
| | | R584) | Fam20c | QRRIQSEQEEAELKQSLEGEAIRNATVNA |
| | | | (R32- | IKEKIKSYGGNETTLGFMVPSYINHRGSP |
| | | | R584) | PKACFVSLITERDSMTQILQSIDEVQVKF |
| | | | | NKNFAYPWVFISQGERLERRGARPSGEP |
| | | | | GCSCAQPAAEVAAPGWAQVRGRPGEPP |
| | | | | AASSAAGDAGWPNKHTLRILQDFSSDPSS |
| | | | | NLSSHSLEKLPPAAEPAERALRGRDPGAL |
| | | | | RPHDPAHRPLLRDPGPRRSESPPGPGGDA |
| | | | | SLLARLFEHPLYRVAVPPLTEEDVLENVN |
| | | | | SDTRLSPKAAENPDWPHAGAEGAEFLSP |
| | | | | GEAAVDSYPNWLKFHIGINRYELYSRHN |
| | | | | PAIEALLHDLSSQRITSVAMKSGGTQLKL |
| | | | | IMTFQNYGQALFKPMKQTREQETPPDFF |
| | | | | YFSDYERHNAEIAAFHLDRILDFRRVPPV |
| | | | | AGRMVNMTKEIRDVTRDKKLWRTFFISP |
| | | | | ANNICFYGECSYYCSTEHALCGKPDQIEG |
| | | | | SLAAFLPDLSLAKRKTWRNPWRRSYHKR |
| | | | | KKAEWEVDPDYCEEVKQTPPYDSSHRIL |
| | | | | DVMDMTIFDFLMGNMDRHHYETFEKFG |
| | | | | NETFIIHLDNGRGFGKYSHDELSILVPLQ |
| | | | | QCCRIRKSTYLRLQLLAKEEYKLSLLMA |
| | | | | ESLRGDQVAPVLYQPHLEALDRRLRVVL |
| | | | | KAVRDCVERNGLHSVVDDDLDTEHRAAS |
| | | | | AR |
| 329 | N/A | Fam20c | Fam20c(R | MRGRPGEPPAASSAAGDAGWPNKHTLRI |
| | | (R64- | 64-R584) | LQDFSSDPSSNLSSHSLEKLPPAAEPAERA |
| | | R584) | | LRGRDPGALRPHDPAHRPLLRDPGPRRS |
| | | | | ESPPGPGGDASLLARLFEHPLYRVAVPPL |
| | | | | TEEDVLFNVNSDTRLSPKAAENPDWPHA |
| | | | | GAEGAEFLSPGEAAVDSYPNWLKFHIGIN |
| | | | | RYELYSRHNPAIEALLHDLSSQRITSVAM |
| | | | | KSGGTQLKLIMTFQNYGQALFKPMKQT |
| | | | | REQETPPDFFYFSDYERHNAEIAAFHLDR |
| | | | | ILDFRRVPPVAGRMVNMTKEIRDVTRDK |
| | | | | KLWRTFFISPANNICFYGECSYYCSTEHA |
| | | | | LCGKPDQIEGSLAAFLPDLSLAKRKTWR |
| | | | | NPWRRSYHKRKKAEWEVDPDYCEEVKQ |
| | | | | TPPYDSSHRILDVMDMTIFDFLMGNMDR |
| | | | | HHYETFEKEGNETFIHLDNGRGFGKYSH |
| | | | | DELSILVPLQQCCRIRKSTYLRLQLLAKE |
| | | | | EYKLSLLMAESLRGDQVAPVLYQPHLEA |
| | | | | LDRRLRVVLKAVRDCVERNGLHSVVDD |
| | | | | DLDTEHRAASAR |
| 330 | ScKRE2 | Fam20c | ScKRE(M | MALFLSKRLLRFTVIAGAVIVLLLTLNSN |
| | (M1-158) | (R64- | 1-158)_ | SRTQQYIPSSISAAFDFTSGSISPEQQVIRG |
| | | R584) | Fam20c | RPGEPPAASSAAGDAGWPNKHTLRILQD |
| | | | (R64- | FSSDPSSNLSSHSLEKLPPAAEPAERALRG |
| | | | R584) | RDPGALRPHDPAHRPLLRDPGPRRSESPP |
| | | | | GPGGDASLLARLFEHPLYRVAVPPLTEE |
| | | | | DVLFNVNSDTRLSPKAAENPDWPHAGAE |
| | | | | GAEFLSPGEAAVDSYPNWLKFHIGINRYE |
| | | | | LYSRHNPAIEALLHDLSSQRITSVAMKSG |
| | | | | GTQLKLIMTFQNYGQALFKPMKQTREQ |
| | | | | ETPPDFFYFSDYERHNAEIAAFHLDRILDF |
| | | | | RRVPPVAGRMVNMTKEIRDVTRDKKLW |
| | | | | RTFFISPANNICFYGECSYYCSTEHALCG |
| | | | | KPDQIEGSLAAFLPDLSLAKRKTWRNPW |
| | | | | RRSYHKRKKAEWEVDPDYCEEVKQTPP |
| | | | | YDSSHRILDVMDMTIFDFLMGNMDRHH |
| | | | | YETFEKFGNETFIIHLDNGRGFGKYSHDE |
| | | | | LSILVPLQQCCRIRKSTYLRLQLLAKEEY |
| | | | | KLSLLMAESLRGDQVAPVLYQPHLEALD |
| | | | | RRLRVVLKAVRDCVERNGLHSVVDDDL |
| | | | | DTEHRAASAR |
| 331 | ScKRE2 | Fam20c | ScKRE2( | MALFLSKRLLRFTVIAGAVIVLLLTLNSN |
| | (M1-S80) | (R64- | M1- | SRTQQYIPSSISAAFDFTSGSISPEQQVISE |
| | | R584) | S80)_Fam | ENDAKKLEQSALNSEASEDSRGRPGEPPA |
| | | | 20c(R64- | ASSAAGDAGWPNKHTLRILQDFSSDPSSN |
| | | | R584) | LSSHSLEKLPPAAEPAERALRGRDPGALR |
| | | | | PHDPAHRPLLRDPGPRRSESPPGPGGDAS |
| | | | | LLARLFEHPLYRVAVPPLTEEDVLFNVNS |
| | | | | DTRLSPKAAENPDWPHAGAEGAEFLSPG |
| | | | | EAAVDSYPNWLKFHIGINRYELYSRHNPA |
| | | | | IEALLHDLSSQRITSVAMKSGGTQLKLIM |
| | | | | TFQNYGQALFKPMKQTREQETPPDFFYF |
| | | | | SDYERHNAEIAAFHLDRILDFRRVPPVAG |
| | | | | RMVNMTKEIRDVTRDKKLWRTFFISPAN |
| | | | | NICFYGECSYYCSTEHALCGKPDQIEGSL |
| | | | | AAFLPDLSLAKRKTWRNPWRRSYHKRK |
| | | | | KAEWEVDPDYCEEVKQTPPYDSSHRILD |
| | | | | VMDMTIFDFLMGNMDRHHYETFEKFGN |
| | | | | ETFIIHLDNGRGFGKYSHDELSILVPLQQ |
| | | | | CCRIRKSTYLRLQLLAKEEYKLSLLMAE |
| | | | | SLRGDQVAPVLYQPHLEALDRRLRVVLK |
| | | | | AVRDCVERNGLHSVVDDDLDTEHRAASA |
| | | | | R |
| 332 | ScKRE2 | Fam20c | ScKRE2( | MALFLSKRLLRFTVIAGAVIVLLLTLNSN |
| | (M1-D102) | (R64- | M1- | SRTQQYIPSSISAAFDFTSGSISPEQQVISE |
| | | R584) | D102)_Fa | ENDAKKLEQSALNSEASEDSEAMDEESK |
| | | | m20c(R64- | ALKAAAEKADAPIDRGRPGEPPAASSAA |
| | | | R584) | GDAGWPNKHTLRILQDFSSDPSSNLSSHS |
| | | | | LEKLPPAAEPAERALRGRDPGALRPHDP |
| | | | | AHRPLLRDPGPRRSESPPGPGGDASLLAR |
| | | | | LFEHPLYRVAVPPLTEEDVLFNVNSDTRL |
| | | | | SPKAAENPDWPHAGAEGAEFLSPGEAAV |
| | | | | DSYPNWLKFHIGINRYELYSRHNPAIEAL |
| | | | | LHDLSSQRITSVAMKSGGTQLKLIMTFQ |
| | | | | NYGQALFKPMKQTREQETPPDFFYFSDY |
| | | | | ERHNAEIAAFHLDRILDFRRVPPVAGRM |
| | | | | VNMTKEIRDVTRDKKLWRTFFISPANNIC |
| | | | | FYGECSYYCSTEHALCGKPDQIEGSLAAF |
| | | | | LPDLSLAKRKTWRNPWRRSYHKRKKAE |
| | | | | WEVDPDYCEEVKQTPPYDSSHRILDVMD |
| | | | | MTIFDFLMGNMDRHHYETFEKFGNETFI |
| | | | | IHLDNGRGFGKYSHDELSILVPLQQCCRI |
| | | | | RKSTYLRLQLLAKEEYKLSLLMAESLRG |
| | | | | DQVAPVLYQPHLEALDRRLRVVLKAVRD |
| | | | | CVERNGLHSVVDDDLDTEHRAASAR |
| 333 | PpKRE2 | Fam20c | PpKRE2( | MVHIGFRSLKAVFILALSSLILYGIVTTED |
| | (M1-G31) | (R64- | M1- | GRGRPGEPPAASSAAGDAGWPNKHTLRI |
| | | R584) | G31)_Fam | LQDFSSDPSSNLSSHSLEKLPPAAEPAERA |
| | | | 20c(R64- | LRGRDPGALRPHDPAHRPLLRDPGPRRS |
| | | | R584) | ESPPGPGGDASLLARLFEHPLYRVAVPPL |
| | | | | TEEDVLFNVNSDTRLSPKAAENPDWPHA |
| | | | | GAEGAEFLSPGEAAVDSYPNWLKFHIGIN |
| | | | | RYELYSRHNPAIEALLHDLSSQRITSVAM |
| | | | | KSGGTQLKLIMTFQNYGQALFKPMKQT |
| | | | | REQETPPDFFYFSDYERHNAEIAAFHLDR |
| | | | | ILDFRRVPPVAGRMVNMTKEIRDVTRDK |
| | | | | KLWRTFFISPANNICFYGECSYYCSTEHA |
| | | | | LCGKPDQIEGSLAAFLPDLSLAKRKTWR |
| | | | | NPWRRSYHKRKKAEWEVDPDYCEEVKQ |
| | | | | TPPYDSSHRILDVMDMTIFDFLMGNMDR |
| | | | | HHYETFEKFGNETFIIHLDNGRGFGKYSH |
| | | | | DELSILVPLQQCCRIRKSTYLRLQLLAKE |
| | | | | EYKLSLLMAESLRGDQVAPVLYQPHLEA |
| | | | | LDRRLRVVLKAVRDCVERNGLHSVVDD |
| | | | | DLDTEHRAASAR |
| 334 | PpKRE2 | Fam20c | PpKRE2( | MVHIGFRSLKAVFILALSSLILYGIVTTED |
| | (M1-D84) | (R64- | M1- | GSRASRYQPPYVNHSQDPLYHSGNSYNR |
| | | R584) | D84)_Fam | ENATFVTLCRNEDLYSHIQSIKKVEDRGR |
| | | | 20c(R64- | PGEPPAASSAAGDAGWPNKHTLRILQDF |
| | | | R584) | SSDPSSNLSSHSLEKLPPAAEPAERALRGR |
| | | | | DPGALRPHDPAHRPLLRDPGPRRSESPPG |
| | | | | PGGDASLLARLFEHPLYRVAVPPLTEEDV |
| | | | | LFNVNSDTRLSPKAAENPDWPHAGAEGA |
| | | | | EFLSPGEAAVDSYPNWLKFHIGINRYELY |
| | | | | SRHNPAIEALLHDLSSQRITSVAMKSGGT |
| | | | | QLKLIMTFQNYGQALFKPMKQTREQET |
| | | | | PPDFFYFSDYERHNAEIAAFHLDRILDER |
| | | | | RVPPVAGRMVNMTKEIRDVTRDKKLWR |
| | | | | TFFISPANNICFYGECSYYCSTEHALCGKP |
| | | | | DQIEGSLAAFLPDLSLAKRKTWRNPWRR |
| | | | | SYHKRKKAEWEVDPDYCEEVKQTPPYDS |
| | | | | SHRILDVMDMTIFDFLMGNMDRHHYETF |
| | | | | EKFGNETFIIHLDNGRGFGKYSHDELSIL |
| | | | | VPLQQCCRIRKSTYLRLQLLAKEEYKLS |
| | | | | LLMAESLRGDQVAPVLYQPHLEALDRRL |
| | | | | RVVLKAVRDCVERNGLHSVVDDDLDTE |
| | | | | HRAASAR |
| 335 | PpKRE2 | Fam20c | PpKRE2( | MVHIGERSLKAVFILALSSLILYGIVTTFD |
| | (M1-H150) | (R64- | M1- | GSRASRYQPPYVNHSQDPLYHSGNSYNR |
| | | R584) | H150)_Fa | ENATFVTLCRNEDLYSIIQSIKKVEDRENN |
| | | | m20c(R64- | KFAYDWVFLNEVPFTDEFKERTSVLISGQ |
| | | | R584) | AKYGLIPKEHWSYPDYIDQERAAESRRQ |
| | | | | LEDQHRGRPGEPPAASSAAGDAGWPNK |
| | | | | HTLRILQDFSSDPSSNLSSHSLEKLPPAAE |
| | | | | PAERALRGRDPGALRPHDPAHRPLLRDP |
| | | | | GPRRSESPPGPGGDASLLARLFEHPLYRV |
| | | | | AVPPLTEEDVLFNVNSDTRLSPKAAENPD |
| | | | | WPHAGAEGAEFLSPGEAAVDSYPNWLKF |
| | | | | HIGINRYELYSRHNPAIEALLHDLSSQRIT |
| | | | | SVAMKSGGTQLKLIMTFQNYGQALFKP |
| | | | | MKQTREQETPPDFFYFSDYERHNAEIAAF |
| | | | | HLDRILDFRRVPPVAGRMVNMTKEIRDV |
| | | | | TRDKKLWRTFFISPANNICFYGECSYYCS |
| | | | | TEHALCGKPDQIEGSLAAFLPDLSLAKRK |
| | | | | TWRNPWRRSYHKRKKAEWEVDPDYCEE |
| | | | | VKQTPPYDSSHRILDVMDMTIFDFLMGN |
| | | | | MDRHHYETFEKFGNETFIIHLDNGRGFG |
| | | | | KYSHDELSILVPLQQCCRIRKSTYLRLQL |
| | | | | LAKEEYKLSLLMAESLRGDQVAPVLYQP |
| | | | | HLEALDRRLRVVLKAVRDCVERNGLHSV |
| | | | | VDDDLDTEHRAASAR |
| 336 | ScMNN2 | Fam20c | ScMNN2( | MLLTKRFSKLFKLTFIVLILCGLFVITNK |
| | (M1-S36) | (R64- | M1- | YMDENTSRGRPGEPPAASSAAGDAGWPN |
| | | R584) | S36)_Fam | KHTLRILQDFSSDPSSNLSSHSLEKLPPAA |
| | | | 20c(R64- | EPAERALRGRDPGALRPHDPAHRPLLRD |
| | | | R584) | PGPRRSESPPGPGGDASLLARLFEHPLYR |
| | | | | VAVPPLTEEDVLFNVNSDTRLSPKAAENP |
| | | | | DWPHAGAEGAEFLSPGEAAVDSYPNWL |
| | | | | KFHIGINRYELYSRANPAIEALLHDLSSQ |
| | | | | RITSVAMKSGGTQLKLIMTFQNYGQALF |
| | | | | KPMKQTREQETPPDFFYFSDYERHNAEI |
| | | | | AAFHLDRILDFRRVPPVAGRMVNMTKEI |
| | | | | RDVTRDKKLWRTFFISPANNICFYGECSY |
| | | | | YCSTEHALCGKPDQIEGSLAAFLPDLSLA |
| | | | | KRKTWRNPWRRSYHKRKKAEWEVDPD |
| | | | | YCEEVKQTPPYDSSHRILDVMDMTIFDFL |
| | | | | MGNMDRHHYETFEKFGNETFIIHLDNGR |
| | | | | GFGKYSHDELSILVPLQQCCRIRKSTYLR |
| | | | | LQLLAKEEYKLSLLMAESLRGDQVAPVL |
| | | | | YQPHLEALDRRLRVVLKAVRDCVERNG |
| | | | | LHSVVDDDLDTEHRAASAR |
| 337 | ScMNN2 | Fam20c | ScMNN2( | MLLTKRFSKLFKLTFIVLILCGLFVITNK |
| | (M1-P97) | (R64- | M1-P97)_ | YMDENTSVKEYKEYLDRYVQSYSNKYSS |
| | | R584) | Fam20c | SSDAASADDSTPLRDNDEAGNEKLKSFYN |
| | | | (R64- | NVFNFLMVDSPRGRPGEPPAASSAAGDA |
| | | | R584) | GWPNKHTLRILQDFSSDPSSNLSSHSLEK |
| | | | | LPPAAEPAERALRGRDPGALRPHDPAHR |
| | | | | PLLRDPGPRRSESPPGPGGDASLLARLFE |
| | | | | HPLYRVAVPPLTEEDVLFNVNSDTRLSPK |
| | | | | AAENPDWPHAGAEGAEFLSPGEAAVDSY |
| | | | | PNWLKFHIGINRYELYSRHNPAIEALLHD |
| | | | | LSSQRITSVAMKSGGTQLKLIMTFQNYG |
| | | | | QALFKPMKQTREQETPPDFFYFSDYERH |
| | | | | NAEIAAFHLDRILDFRRVPPVAGRMVNM |
| | | | | TKEIRDVTRDKKLWRTFFISPANNICFYG |
| | | | | ECSYYCSTEHALCGKPDQIEGSLAAFLPD |
| | | | | LSLAKRKTWRNPWRRSYHKRKKAEWE |
| | | | | VDPDYCEEVKQTPPYDSSHRILDVMDMT |
| | | | | IFDFLMGNMDRHHYETFEKFGNETFIIHL |
| | | | | DNGRGFGKYSHDELSILVPLQQCCRIRKS |
| | | | | TYLRLQLLAKEEYKLSLLMAESLRGDQV |
| | | | | APVLYQPHLEALDRRLRVVLKAVRDCVE |
| | | | | RNGLHSVVDDDLDTEHRAASAR |
| 338 | ScMNN2 | Fam20c | ScMNN2( | MLLTKRFSKLFKLTFIVLILCGLFVITNK |
| | (M1-S150) | (R64- | M1-S150)_ | YMDENTSVKEYKEYLDRYVQSYSNKYSS |
| | | R584) | Fam20c | SSDAASADDSTPLRDNDEAGNEKLKSFYN |
| | | | (R64-R584) | NVFNFLMVDSPKGSTAKQYNEACLLKGD |
| | | | | IGDRPDHYKDLYKLSAKELSKCLELSPDE |
| | | | | VASLTKSRGRPGEPPAASSAAGDAGWPN |
| | | | | KHTLRILQDFSSDPSSNLSSHSLEKLPPAA |
| | | | | EPAERALRGRDPGALRPHDPAHRPLLRD |
| | | | | PGPRRSESPPGPGGDASLLARLFEHPLYR |
| | | | | VAVPPLTEEDVLFNVNSDTRLSPKAAENP |
| | | | | DWPHAGAEGAEFLSPGEAAVDSYPNWL |
| | | | | KFHIGINRYELYSRUNPAIEALLHDLSSQ |
| | | | | RITSVAMKSGGTQLKLIMTFQNYGQALF |
| | | | | KPMKQTREQETPPDFFYFSDYERHNAEI |
| | | | | AAFHLDRILDFRRVPPVAGRMVNMTKEI |
| | | | | RDVTRDKKLWRTFFISPANNICFYGECSY |
| | | | | YCSTEHALCGKPDQIEGSLAAFLPDLSLA |
| | | | | KRKTWRNPWRRSYHKRKKAEWEVDPD |
| | | | | YCEEVKQTPPYDSSHRILDVMDMTIFDFL |
| | | | | MGNMDRHHYETFEKFGNETFIIHLDNGR |
| | | | | GFGKYSHDELSILVPLQQCCRIRKSTYLR |
| | | | | LQLLAKEEYKLSLLMAESLRGDQVAPVL |
| | | | | YQPHLEALDRRLRVVLKAVRDCVERNG |
| | | | | LHSVVDDDLDTEHRAASAR |
| 339 | ScMNN1 | Fam20c | ScMNN1( | MLALRRFILNQRSLRSCTIPILVGALIILV |
| | (M1-A42) | (R64- | M1-A42)_ | LFQLVTHRNDARGRPGEPPAASSAAGDA |
| | | R584) | Fam20c | GWPNKHTLRILQDFSSDPSSNLSSHSLEK |
| | | | (R64- | LPPAAEPAERALRGRDPGALRPHDPAHR |
| | | | R584) | PLLRDPGPRRSESPPGPGGDASLLARLFE |
| | | | | HPLYRVAVPPLTEEDVLFNVNSDTRLSPK |
| | | | | AAENPDWPHAGAEGAEFLSPGEAAVDSY |
| | | | | PNWLKFHIGINRYELYSRHNPAIEALLHD |
| | | | | LSSQRITSVAMKSGGTQLKLIMTFQNYG |
| | | | | QALFKPMKQTREQETPPDFFYFSDYERH |
| | | | | NAEIAAFHLDRILDFRRVPPVAGRMVNM |
| | | | | TKEIRDVTRDKKLWRTFFISPANNICFYG |
| | | | | ECSYYCSTEHALCGKPDQIEGSLAAFLPD |
| | | | | LSLAKRKTWRNPWRRSYHKRKKAEWE |
| | | | | VDPDYCEEVKQTPPYDSSHRILDVMDMT |
| | | | | IFDFLMGNMDRHHYETFEKFGNETFIIHL |
| | | | | DNGRGFGKYSHDELSILVPLQQCCRIRKS |
| | | | | TYLRLQLLAKEEYKLSLLMAESLRGDQV |
| | | | | APVLYQPHLEALDRRLRVVLKAVRDCVE |
| | | | | RNGLHSVVDDDLDTEHRAASAR |
| 340 | ScMNN1 | Fam20c | ScMNN1( | MLALRRFILNQRSLRSCTIPILVGALIILV |
| | (M1-Q93) | (R64- | M1-Q93)_ | LFQLVTHRNDALIRSSNVNSTNKKTLKDA |
| | | R584) | Fam20c | DPKVLIEAFGSPEVDPVDTIPVSPLELVPF |
| | | | (R64- | YDQRGRPGEPPAASSAAGDAGWPNKHT |
| | | | R584) | LRILQDFSSDPSSNLSSHSLEKLPPAAEPA |
| | | | | ERALRGRDPGALRPHDPAHRPLLRDPGP |
| | | | | RRSESPPGPGGDASLLARLFEHPLYRVAV |
| | | | | PPLTEEDVLFNVNSDTRLSPKAAENPDWP |
| | | | | HAGAEGAEFLSPGEAAVDSYPNWLKFHI |
| | | | | GINRYELYSRHNPAIEALLHDLSSQRITSV |
| | | | | AMKSGGTQLKLIMTFQNYGQALFKPMK |
| | | | | QTREQETPPDFFYFSDYERHNAEIAAFHL |
| | | | | DRILDFRRVPPVAGRMVNMTKEIRDVTR |
| | | | | DKKLWRTFFISPANNICFYGECSYYCSTE |
| | | | | HALCGKPDQIEGSLAAFLPDLSLAKRKT |
| | | | | WRNPWRRSYHKRKKAEWEVDPDYCEE |
| | | | | VKQTPPYDSSHRILDVMDMTIFDFLMGN |
| | | | | MDRHHYETFEKFGNETFIIHLDNGRGFG |
| | | | | KYSHDELSILVPLQQCCRIRKSTYLRLQL |
| | | | | LAKEEYKLSLLMAESLRGDQVAPVLYQP |
| | | | | HLEALDRRLRVVLKAVRDCVERNGLHSV |
| | | | | VDDDLDTEHRAASAR |
| 341 | ScMNN1 | Fam20c | ScMNN1( | MLALRRFILNQRSLRSCTIPILVGALIIILV |
| | (M1-G153) | (R64- | M1-G153)_ | LFQLVTHRNDALIRSSNVNSTNKKTLKDA |
| | | R584) | Fam20c | DPKVLIEAFGSPEVDPVDTIPVSPLELVPF |
| | | | (R64- | YDQSIDTKRSSSWLINKKGYYKHFNELSL |
| | | | R584) | TDRCKFYFRTLYTLDDEWTNSVKKLEYS |
| | | | | INDNEGRGRPGEPPAASSAAGDAGWPNK |
| | | | | HTLRILQDFSSDPSSNLSSHSLEKLPPAAE |
| | | | | PAERALRGRDPGALRPHDPAHRPLLRDP |
| | | | | GPRRSESPPGPGGDASLLARLFEHPLYRV |
| | | | | AVPPLTEEDVLFNVNSDTRLSPKAAENPD |
| | | | | WPHAGAEGAEFLSPGEAAVDSYPNWLKF |
| | | | | HIGINRYELYSRHNPAIEALLHDLSSQRIT |
| | | | | SVAMKSGGTQLKLIMTFQNYGQALFKP |
| | | | | MKQTREQETPPDFFYFSDYERHNAEIAAF |
| | | | | HLDRILDFRRVPPVAGRMVNMTKEIRDV |
| | | | | TRDKKLWRTFFISPANNICFYGECSYYCS |
| | | | | TEHALCGKPDQIEGSLAAFLPDLSLAKRK |
| | | | | TWRNPWRRSYHKRKKAEWEVDPDYCEE |
| | | | | VKQTPPYDSSHRILDVMDMTIFDFLMGN |
| | | | | MDRHHYETFEKFGNETFIIHLDNGRGFG |
| | | | | KYSHDELSILVPLQQCCRIRKSTYLRLQL |
| | | | | LAKEEYKLSLLMAESLRGDQVAPVLYQP |
| | | | | HLEALDRRLRVVLKAVRDCVERNGLHSV |
| | | | | VDDDLDTEHRAASAR |
| 342 | ScMNN6 | Fam20c | ScMNN6( | MHVLLSKKIARFLLISFVFVLALMVTINH |
| | (M1-P30) | (R64- | M1-P30)_ | PRGRPGEPPAASSAAGDAGWPNKHTLRI |
| | | R584) | Fam20c | LQDFSSDPSSNLSSHSLEKLPPAAEPAERA |
| | | | (R64- | LRGRDPGALRPHDPAHRPLLRDPGPRRS |
| | | | R584) | ESPPGPGGDASLLARLFEHPLYRVAVPPL |
| | | | | TEEDVLFNVNSDTRLSPKAAENPDWPHA |
| | | | | GAEGAEFLSPGEAAVDSYPNWLKFHIGIN |
| | | | | RYELYSRHNPAIEALLHDLSSQRITSVAM |
| | | | | KSGGTQLKLIMTFQNYGQALFKPMKQT |
| | | | | REQETPPDFFYFSDYERHNAEIAAFHLDR |
| | | | | ILDFRRVPPVAGRMVNMTKEIRDVTRDK |
| | | | | KLWRTFFISPANNICFYGECSYYCSTEHA |
| | | | | LCGKPDQIEGSLAAFLPDLSLAKRKTWR |
| | | | | NPWRRSYHKRKKAEWEVDPDYCEEVKQ |
| | | | | TPPYDSSHRILDVMDMTIFDFLMGNMDR |
| | | | | HHYETFEKFGNETFIHLDNGRGFGKYSH |
| | | | | DELSILVPLQQCCRIRKSTYLRLQLLAKE |
| | | | | EYKLSLLMAESLRGDQVAPVLYQPHLEA |
| | | | | LDRRLRVVLKAVRDCVERNGLHSVVDD |
| | | | | DLDTEHRAASAR |
| 343 | ScMNN6 | Fam20c | ScMNN6 | MHVLLSKKIARFLLISFVFVLALMVTINH |
| | (M1-V85) | (R64- | (M1-V85)_ | PKTKQMSEQYVTPYLPKSLQPIAKISAEE |
| | | R584) | Fam20c | QRRIQSEQEEAELKQSLEGEAIRNATVRG |
| | | | (R64- | RPGEPPAASSAAGDAGWPNKHTLRILQD |
| | | | R584) | FSSDPSSNLSSHSLEKLPPAAEPAERALRG |
| | | | | RDPGALRPHDPAHRPLLRDPGPRRSESPP |
| | | | | GPGGDASLLARLFEHPLYRVAVPPLTEE |
| | | | | DVLFNVNSDTRLSPKAAENPDWPHAGAE |
| | | | | GAEFLSPGEAAVDSYPNWLKFHIGINRYE |
| | | | | LYSRANPAIEALLHDLSSQRITSVAMKSG |
| | | | | GTQLKLIMTFQNYGQALFKPMKQTREQ |
| | | | | ETPPDFFYFSDYERHNAEIAAFHLDRILDF |
| | | | | RRVPPVAGRMVNMTKEIRDVTRDKKLW |
| | | | | RTFFISPANNICFYGECSYYCSTEHALCG |
| | | | | KPDQIEGSLAAFLPDLSLAKRKTWRNPW |
| | | | | RRSYHKRKKAEWEVDPDYCEEVKQTPP |
| | | | | YDSSHRILDVMDMTIFDFLMGNMDRHH |
| | | | | YETFEKFGNETFIIHLDNGRGFGKYSHDE |
| | | | | LSILVPLQQCCRIRKSTYLRLQLLAKEEY |
| | | | | KLSLLMAESLRGDQVAPVLYQPHLEALD |
| | | | | RRLRVVLKAVRDCVERNGLHSVVDDDL |
| | | | | DTEHRAASAR |
| 344 | ScMNN6 | Fam20c | ScMNN6( | MHVLLSKKIARFLLISFVFVLALMVTINH |
| | (M1-E160) | (R64- | M1-E160)_ | PKTKQMSEQYVTPYLPKSLQPIAKISAEE |
| | | R584) | Fam20c | QRRIQSEQEEAELKQSLEGEAIRNATVNA |
| | | | (R64- | IKEKIKSYGGNETTLGFMVPSYINHRGSP |
| | | | R584) | PKACFVSLITERDSMTQILQSIDEVQVKF |
| | | | | NKNFAYPWVFISQGERGRPGEPPAASSAA |
| | | | | GDAGWPNKHTLRILQDFSSDPSSNLSSHS |
| | | | | LEKLPPAAEPAERALRGRDPGALRPHDP |
| | | | | AHRPLLRDPGPRRSESPPGPGGDASLLAR |
| | | | | LFEHPLYRVAVPPLTEEDVLFNVNSDTRL |
| | | | | SPKAAENPDWPHAGAEGAEFLSPGEAAV |
| | | | | DSYPNWLKFHIGINRYELYSRANPAIEAL |
| | | | | LHDLSSQRITSVAMKSGGTQLKLIMTFQ |
| | | | | NYGQALFKPMKQTREQETPPDFFYFSDY |
| | | | | ERHNAEIAAFHLDRILDFRRVPPVAGRM |
| | | | | VNMTKEIRDVTRDKKLWRTFFISPANNIC |
| | | | | FYGECSYYCSTEHALCGKPDQIEGSLAAF |
| | | | | LPDLSLAKRKTWRNPWRRSYHKRKKAE |
| | | | | WEVDPDYCEEVKQTPPYDSSHRILDVMD |
| | | | | MTIFDFLMGNMDRHHYETFEKFGNETFI |
| | | | | IHLDNGRGFGKYSHDELSILVPLQQCCRI |
| | | | | RKSTYLRLQLLAKEEYKLSLLMAESLRG |
| | | | | DQVAPVLYQPHLEALDRRLRVVLKAVRD |
| | | | | CVERNGLHSVVDDDLDTEHRAASAR |
| 345 | N/A | Fam20c | Fam20c | MDFSSDPSSNLSSHSLEKLPPAAEPAERAL |
| | | (D93- | (D93- | RGRDPGALRPHDPAHRPLLRDPGPRRSE |
| | | R584) | R584) | SPPGPGGDASLLARLFEHPLYRVAVPPLT |
| | | | | EEDVLFNVNSDTRLSPKAAENPDWPHAG |
| | | | | AEGAEFLSPGEAAVDSYPNWLKFHIGINR |
| | | | | YELYSRHNPAIEALLHDLSSQRITSVAMK |
| | | | | SGGTQLKLIMTFQNYGQALFKPMKQTR |
| | | | | EQETPPDFFYFSDYERHNAEIAAFHLDRI |
| | | | | LDFRRVPPVAGRMVNMTKEIRDVTRDK |
| | | | | KLWRTFFISPANNICFYGECSYYCSTEHA |
| | | | | LCGKPDQIEGSLAAFLPDLSLAKRKTWR |
| | | | | NPWRRSYHKRKKAEWEVDPDYCEEVKQ |
| | | | | TPPYDSSHRILDVMDMTIFDFLMGNMDR |
| | | | | HHYETFEKEGNETFIIHLDNGRGFGKYSH |
| | | | | DELSILVPLQQCCRIRKSTYLRLQLLAKE |
| | | | | EYKLSLLMAESLRGDQVAPVLYQPHLEA |
| | | | | LDRRLRVVLKAVRDCVERNGLHSVVDD |
| | | | | DLDTEHRAASAR |
| 346 | ScKRE2 | Fam20c | ScKRE(M | MALFLSKRLLRFTVIAGAVIVLLLTLNSN |
| | (M1-158) | (D93- | 158)_ | SRTQQYIPSSISAAFDFTSGSISPEQQVIDF |
| | | R584) | Fam20c | SSDPSSNLSSHSLEKLPPAAEPAERALRGR |
| | | | (D93-R584) | DPGALRPHDPAHRPLLRDPGPRRSESPPG |
| | | | | PGGDASLLARLFEHPLYRVAVPPLTEEDV |
| | | | | LFNVNSDTRLSPKAAENPDWPHAGAEGA |
| | | | | EFLSPGEAAVDSYPNWLKFHIGINRYELY |
| | | | | SRHNPAIEALLHDLSSQRITSVAMKSGGT |
| | | | | QLKLIMTFQNYGQALFKPMKQTREQET |
| | | | | PPDFFYFSDYERHNAEIAAFHLDRILDFR |
| | | | | RVPPVAGRMVNMTKEIRDVTRDKKLWR |
| | | | | TFFISPANNICFYGECSYYCSTEHALCGKP |
| | | | | DQIEGSLAAFLPDLSLAKRKTWRNPWRR |
| | | | | SYHKRKKAEWEVDPDYCEEVKQTPPYDS |
| | | | | SHRILDVMDMTIFDFLMGNMDRHHYETF |
| | | | | EKFGNETFIIHLDNGRGFGKYSHDELSIL |
| | | | | VPLQQCCRIRKSTYLRLQLLAKEEYKLS |
| | | | | LLMAESLRGDQVAPVLYQPHLEALDRRL |
| | | | | RVVLKAVRDCVERNGLHSVVDDDLDTE |
| | | | | HRAASAR |
| 347 | ScKRE2 | Fam20c | ScKRE2( | MALFLSKRLLRFTVIAGAVIVLLLTLNSN |
| | (M1-S80) | (D93- | M1-S80)_ | SRTQQYIPSSISAAFDFTSGSISPEQQVISE |
| | | R584) | Fam20c | ENDAKKLEQSALNSEASEDSDFSSDPSSNL |
| | | | (D93- | SSHSLEKLPPAAEPAERALRGRDPGALRP |
| | | | R584) | HDPAHRPLLRDPGPRRSESPPGPGGDASL |
| | | | | LARLFEHPLYRVAVPPLTEEDVLFNVNSD |
| | | | | TRLSPKAAENPDWPHAGAEGAEFLSPGE |
| | | | | AAVDSYPNWLKFHIGINRYELYSRHNPAI |
| | | | | EALLHDLSSQRITSVAMKSGGTQLKLIM |
| | | | | TFQNYGQALFKPMKQTREQETPPDFFYF |
| | | | | SDYERHNAEIAAFHLDRILDFRRVPPVAG |
| | | | | RMVNMTKEIRDVTRDKKLWRTFFISPAN |
| | | | | NICFYGECSYYCSTEHALCGKPDQIEGSL |
| | | | | AAFLPDLSLAKRKTWRNPWRRSYHKRK |
| | | | | KAEWEVDPDYCEEVKQTPPYDSSHRILD |
| | | | | VMDMTIFDFLMGNMDRHHYETFEKFGN |
| | | | | ETFIIHLDNGRGFGKYSHDELSILVPLQQ |
| | | | | CCRIRKSTYLRLQLLAKEEYKLSLLMAE |
| | | | | SLRGDQVAPVLYQPHLEALDRRLRVVLK |
| | | | | AVRDCVERNGLHSVVDDDLDTEHRAASA |
| | | | | R |
| 348 | ScKRE2 | Fam20c | ScKRE2 | MALFLSKRLLRFTVIAGAVIVLLLTLNSN |
| | (M1-D102) | (D93- | (M1-D102)_ | SRTQQYIPSSISAAFDFTSGSISPEQQVISE |
| | | R584) | Fam20c | ENDAKKLEQSALNSEASEDSEAMDEESK |
| | | | (D93- | ALKAAAEKADAPIDDFSSDPSSNLSSHSLE |
| | | | R584) | KLPPAAEPAERALRGRDPGALRPHDPAH |
| | | | | RPLLRDPGPRRSESPPGPGGDASLLARLF |
| | | | | EHPLYRVAVPPLTEEDVLFNVNSDTRLSP |
| | | | | KAAENPDWPHAGAEGAEFLSPGEAAVDS |
| | | | | YPNWLKFHIGINRYELYSRHNPAIEALLH |
| | | | | DLSSQRITSVAMKSGGTQLKLIMTFQNY |
| | | | | GQALFKPMKQTREQETPPDFFYFSDYER |
| | | | | HNAEIAAFHLDRILDFRRVPPVAGRMVN |
| | | | | MTKEIRDVTRDKKLWRTFFISPANNICFY |
| | | | | GECSYYCSTEHALCGKPDQIEGSLAAFLP |
| | | | | DLSLAKRKTWRNPWRRSYHKRKKAEW |
| | | | | EVDPDYCEEVKQTPPYDSSHRILDVMDM. |
| | | | | TIFDFLMGNMDRHHYETFEKFGNETFIH |
| | | | | LDNGRGFGKYSHDELSILVPLQQCCRIRK |
| | | | | STYLRLQLLAKEEYKLSLLMAESLRGDQ |
| | | | | VAPVLYQPHLEALDRRLRVVLKAVRDCV |
| | | | | ERNGLHSVVDDDLDTEHRAASAR |
| 349 | PpKRE2 | Fam20c | PpKRE2( | MVHIGFRSLKAVFILALSSLILYGIVTTFD |
| | (M1-G31) | (D93- | M1-G31)_ | GDFSSDPSSNLSSHSLEKLPPAAEPAERAL |
| | | R584) | Fam20c | RGRDPGALRPHDPAHRPLLRDPGPRRSE |
| | | | (D93- | SPPGPGGDASLLARLFEHPLYRVAVPPLT |
| | | | R584) | EEDVLFNVNSDTRLSPKAAENPDWPHAG |
| | | | | AEGAEFLSPGEAAVDSYPNWLKFHIGINR |
| | | | | YELYSRHNPAIEALLHDLSSQRITSVAMK |
| | | | | SGGTQLKLIMTFQNYGQALFKPMKQTR |
| | | | | EQETPPDFFYFSDYERHNAEIAAFHLDRI |
| | | | | LDFRRVPPVAGRMVNMTKEIRDVTRDK |
| | | | | KLWRTFFISPANNICFYGECSYYCSTEHA |
| | | | | LCGKPDQIEGSLAAFLPDLSLAKRKTWR |
| | | | | NPWRRSYHKRKKAEWEVDPDYCEEVKQ |
| | | | | TPPYDSSHRILDVMDMTIFDFLMGNMDR |
| | | | | HHYETFEKFGNETFIIHLDNGRGFGKYSH |
| | | | | DELSILVPLQQCCRIRKSTYLRLQLLAKE |
| | | | | EYKLSLLMAESLRGDQVAPVLYQPHLEA |
| | | | | LDRRLRVVLKAVRDCVERNGLHSVVDD |
| | | | | DLDTEHRAASAR |
| 350 | PpKRE2 | Fam20c | PpKRE2( | MVHIGFRSLKAVFILALSSLILYGIVTTFD |
| | (M1-D84) | (D93- | M1-D84)_ | GSRASRYQPPYVNHSQDPLYHSGNSYNR |
| | | R584) | Fam20c | ENATFVTLCRNEDLYSIIQSIKKVEDDESS |
| | | | (D93- | DPSSNLSSHSLEKLPPAAEPAERALRGRD |
| | | | R584) | PGALRPHDPAHRPLLRDPGPRRSESPPGP |
| | | | | GGDASLLARLFEHPLYRVAVPPLTEEDV |
| | | | | LFNVNSDTRLSPKAAENPDWPHAGAEGA |
| | | | | EFLSPGEAAVDSYPNWLKFHIGINRYELY |
| | | | | SRHNPAIEALLHDLSSQRITSVAMKSGGT |
| | | | | QLKLIMTFQNYGQALFKPMKQTREQET |
| | | | | PPDFFYFSDYERHNAEIAAFHLDRILDFR |
| | | | | RVPPVAGRMVNMTKEIRDVTRDKKLWR |
| | | | | TFFISPANNICFYGECSYYCSTEHALCGKP |
| | | | | DQIEGSLAAFLPDLSLAKRKTWRNPWRR |
| | | | | SYHKRKKAEWEVDPDYCEEVKQTPPYDS |
| | | | | SHRILDVMDMTIFDFLMGNMDRHHYETF |
| | | | | EKFGNETFIIHLDNGRGFGKYSHDELSIL |
| | | | | VPLQQCCRIRKSTYLRLQLLAKEEYKLS |
| | | | | LLMAESLRGDQVAPVLYQPHLEALDRRL |
| | | | | RVVLKAVRDCVERNGLHSVVDDDLDTE |
| | | | | HRAASAR |
| 351 | PpKRE2 | Fam20c | PpKRE2( | MVHIGFRSLKAVFILALSSLILYGIVITFD |
| | (M1-H150) | (D93- | M1-H150)_ | GSRASRYQPPYVNHSQDPLYHSGNSYNR |
| | | R584) | Fam20c | ENATEVTLCRNEDLYSIIQSIKKVEDRENN |
| | | | (D93- | KFAYDWVFLNEVPFTDEFKERTSVLISGQ |
| | | | R584) | AKYGLIPKEHWSYPDYIDQERAAESRRQ |
| | | | | LEDQHDFSSDPSSNLSSHSLEKLPPAAEPA |
| | | | | ERALRGRDPGALRPHDPAHRPLLRDPGP |
| | | | | RRSESPPGPGGDASLLARLFEHPLYRVAV |
| | | | | PPLTEEDVLFNVNSDTRLSPKAAENPDWP |
| | | | | HAGAEGAEFLSPGEAAVDSYPNWLKFHI |
| | | | | GINRYELYSRHNPAIEALLHDLSSQRITSV |
| | | | | AMKSGGTQLKLIMTFQNYGQALFKPMK |
| | | | | QTREQETPPDFFYFSDYERHNAEIAAFHL |
| | | | | DRILDFRRVPPVAGRMVNMTKEIRDVTR |
| | | | | DKKLWRTFFISPANNICFYGECSYYCSTE |
| | | | | HALCGKPDQIEGSLAAFLPDLSLAKRKT |
| | | | | WRNPWRRSYHKRKKAEWEVDPDYCEE |
| | | | | VKQTPPYDSSHRILDVMDMTIFDFLMGN |
| | | | | MDRHHYETFEKFGNETFIIHLDNGRGFG |
| | | | | KYSHDELSILVPLQQCCRIRKSTYLRLQL |
| | | | | LAKEEYKLSLLMAESLRGDQVAPVLYQP |
| | | | | HLEALDRRLRVVLKAVRDCVERNGLHSV |
| | | | | VDDDLDTEHRAASAR |
| 352 | ScMNN2 | Fam20c | ScMNN2 | MLLTKRFSKLFKLTFIVLILCGLFVITNK |
| | (M1-S36) | (D93- | (M1-S36)_ | YMDENTSDFSSDPSSNLSSHSLEKLPPAAE |
| | | R584) | Fam20c | PAERALRGRDPGALRPHDPAHRPLLRDP |
| | | | (D93- | GPRRSESPPGPGGDASLLARLFEHPLYRV |
| | | | R584) | AVPPLTEEDVLFNVNSDTRLSPKAAENPD |
| | | | | WPHAGAEGAEFLSPGEAAVDSYPNWLKF |
| | | | | HIGINRYELYSRHNPAIEALLHDLSSQRIT |
| | | | | SVAMKSGGTQLKLIMTFQNYGQALFKP |
| | | | | MKQTREQETPPDFFYFSDYERHNAEIAAF |
| | | | | HLDRILDFRRVPPVAGRMVNMTKEIRDV |
| | | | | TRDKKLWRTFFISPANNICFYGECSYYCS |
| | | | | TEHALCGKPDQIEGSLAAFLPDLSLAKRK |
| | | | | TWRNPWRRSYHKRKKAEWEVDPDYCEE |
| | | | | VKQTPPYDSSHRILDVMDMTIFDFLMGN |
| | | | | MDRHHYETFEKFGNETFIIHLDNGRGFG |
| | | | | KYSHDELSILVPLQQCCRIRKSTYLRLQL |
| | | | | LAKEEYKLSLLMAESLRGDQVAPVLYQP |
| | | | | HLEALDRRLRVVLKAVRDCVERNGLHSV |
| | | | | VDDDLDTEHRAASAR |
| 353 | ScMNN2 | Fam20c | ScMNN2 | MLLTKRFSKLFKLTFIVLILCGLFVITNK |
| | (M1-P97) | (D93- | (M1-P97)_ | YMDENTSVKEYKEYLDRYVQSYSNKYSS |
| | | R584) | Fam20c | SSDAASADDSTPLRDNDEAGNEKLKSFYN |
| | | | (D93- | NVFNFLMVDSPDFSSDPSSNLSSHSLEKLP |
| | | | R584) | PAAEPAERALRGRDPGALRPHDPAHRPL |
| | | | | LRDPGPRRSESPPGPGGDASLLARLFEHP |
| | | | | LYRVAVPPLTEEDVLFNVNSDTRLSPKAA |
| | | | | ENPDWPHAGAEGAEFLSPGEAAVDSYPN |
| | | | | WLKFHIGINRYELYSRHNPAIEALLHDLS |
| | | | | SQRITSVAMKSGGTQLKLIMTFQNYGQA |
| | | | | LFKPMKQTREQETPPDFFYFSDYERHNA |
| | | | | EIAAFHLDRILDFRRVPPVAGRMVNMTK |
| | | | | EIRDVTRDKKLWRTFFISPANNICFYGEC |
| | | | | SYYCSTEHALCGKPDQIEGSLAAFLPDLS |
| | | | | LAKRKTWRNPWRRSYHKRKKAEWEVD |
| | | | | PDYCEEVKQTPPYDSSHRILDVMDMTIFD |
| | | | | FLMGNMDRHHYETFEKFGNETFIIHLDN |
| | | | | GRGFGKYSHDELSILVPLQQCCRIRKSTY |
| | | | | LRLQLLAKEEYKLSLLMAESLRGDQVAP |
| | | | | VLYQPHLEALDRRLRVVLKAVRDCVERN |
| | | | | GLHSVVDDDLDTEHRAASAR |
| 354 | ScMNN2 | Fam20c | ScMNN2 | MLLTKRFSKLFKLTFIVLILCGLFVITNK |
| | (M1-S150) | (D93- | (M1-S150)_ | YMDENTSVKEYKEYLDRYVQSYSNKYSS |
| | | R584) | Fam20c | SSDAASADDSTPLRDNDEAGNEKLKSFYN |
| | | | (D93- | NVFNFLMVDSPKGSTAKQYNEACLLKGD |
| | | | R584) | IGDRPDHYKDLYKLSAKELSKCLELSPDE |
| | | | | VASLTKSDFSSDPSSNLSSHSLEKLPPAAE |
| | | | | PAERALRGRDPGALRPHDPAHRPLLRDP |
| | | | | GPRRSESPPGPGGDASLLARLFEHPLYRV |
| | | | | AVPPLTEEDVLFNVNSDTRLSPKAAENPD |
| | | | | WPHAGAEGAEFLSPGEAAVDSYPNWLKF |
| | | | | HIGINRYELYSRHNPAIEALLHDLSSQRIT |
| | | | | SVAMKSGGTQLKLIMTFQNYGQALFKP |
| | | | | MKQTREQETPPDFFYFSDYERHNAEIAAF |
| | | | | HLDRILDFRRVPPVAGRMVNMTKEIRDV |
| | | | | TRDKKLWRTFFISPANNICFYGECSYYCS |
| | | | | TEHALCGKPDQIEGSLAAFLPDLSLAKRK |
| | | | | TWRNPWRRSYHKRKKAEWEVDPDYCEE |
| | | | | VKQTPPYDSSHRILDVMDMTIFDFLMGN |
| | | | | MDRHHYETFEKFGNETFIIHLDNGRGFG |
| | | | | KYSHDELSILVPLQQCCRIRKSTYLRLQL |
| | | | | LAKEEYKLSLLMAESLRGDQVAPVLYQP |
| | | | | HLEALDRRLRVVLKAVRDCVERNGLHSV |
| | | | | VDDDLDTEHRAASAR |
| 355 | ScMNN1 | Fam20c | ScMINN1( | MLALRRFILNQRSLRSCTIPILVGALIILV |
| | (M1-A42) | (D93- | M1-A42)_ | LFQLVTHRNDADFSSDPSSNLSSHSLEKLP |
| | | R584) | Fam20c | PAAEPAERALRGRDPGALRPHDPAHRPL |
| | | | (D93- | LRDPGPRRSESPPGPGGDASLLARLFEHP |
| | | | R584) | LYRVAVPPLTEEDVLENVNSDTRLSPKAA |
| | | | | ENPDWPHAGAEGAEFLSPGEAAVDSYPN |
| | | | | WLKFHIGINRYELYSRANPAIEALLHDLS |
| | | | | SQRITSVAMKSGGTQLKLIMTFQNYGQA |
| | | | | LFKPMKQTREQETPPDFFYFSDYERHNA |
| | | | | EIAAFHLDRILDFRRVPPVAGRMVNMTK |
| | | | | EIRDVTRDKKLWRTFFISPANNICFYGEC |
| | | | | SYYCSTEHALCGKPDQIEGSLAAFLPDLS |
| | | | | LAKRKTWRNPWRRSYHKRKKAEWEVD |
| | | | | PDYCEEVKQTPPYDSSHRILDVMDMTIFD |
| | | | | FLMGNMDRHHYETFEKFGNETFIIHLDN |
| | | | | GRGFGKYSHDELSILVPLQQCCRIRKSTY |
| | | | | LRLQLLAKEEYKLSLLMAESLRGDQVAP |
| | | | | VLYQPHLEALDRRLRVVLKAVRDCVERN |
| | | | | GLHSVVDDDLDTEHRAASAR |
| 356 | ScMNN1 | Fam20c | ScMNN1( | MLALRRFILNQRSLRSCTIPILVGALIILV |
| | (M1-Q93) | (D93- | M1-Q93)_ | LFQLVTHRNDALIRSSNVNSTNKKTLKDA |
| | | R584) | Fam20c | DPKVLIEAFGSPEVDPVDTIPVSPLELVPF |
| | | | (D93- | YDQDFSSDPSSNLSSHSLEKLPPAAEPAER |
| | | | R584) | ALRGRDPGALRPHDPAHRPLLRDPGPRR |
| | | | | SESPPGPGGDASLLARLFEHPLYRVAVPP |
| | | | | LTEEDVLFNVNSDTRLSPKAAENPDWPH |
| | | | | AGAEGAEFLSPGEAAVDSYPNWLKFHIGI |
| | | | | NRYELYSRHNPAIEALLHDLSSQRITSVA |
| | | | | MKSGGTQLKLIMTFQNYGQALFKPMKQ |
| | | | | TREQETPPDFFYFSDYERHNAEIAAFHLD |
| | | | | RILDFRRVPPVAGRMVNMTKEIRDVTRD |
| | | | | KKLWRTFFISPANNICFYGECSYYCSTEH |
| | | | | ALCGKPDQIEGSLAAFLPDLSLAKRKTW |
| | | | | RNPWRRSYHKRKKAEWEVDPDYCEEVK |
| | | | | QTPPYDSSHRILDVMDMTIFDFLMGNMD |
| | | | | RHHYETFEKFGNETFIIHLDNGRGFGKYS |
| | | | | HDELSILVPLQQCCRIRKSTYLRLQLLAK |
| | | | | EEYKLSLLMAESLRGDQVAPVLYQPHLE |
| | | | | ALDRRLRVVLKAVRDCVERNGLASVVD |
| | | | | DDLDTEHRAASAR |
| 357 | ScMNN1 | Fam20c | ScMNN1( | MLALRRFILNQRSLRSCTIPILVGALIILV |
| | (M1-G153) | (D93- | M1-G153)_ | LFQLVTHRNDALIRSSNVNSTNKKTLKDA |
| | | R584) | Fam20c | DPKVLIEAFGSPEVDPVDTIPVSPLELVPF |
| | | | (D93- | YDQSIDTKRSSSWLINKKGYYKHFNELSL |
| | | | R584) | TDRCKFYFRTLYTLDDEWTNSVKKLEYS |
| | | | | INDNEGDFSSDPSSNLSSHSLEKLPPAAEP |
| | | | | AERALRGRDPGALRPHDPAHRPLLRDPG |
| | | | | PRRSESPPGPGGDASLLARLFEHPLYRVA |
| | | | | VPPLTEEDVLFNVNSDTRLSPKAAENPD |
| | | | | WPHAGAEGAEFLSPGEAAVDSYPNWLKF |
| | | | | HIGINRYELYSRHNPAIEALLHDLSSQRIT |
| | | | | SVAMKSGGTQLKLIMTFQNYGQALFKP |
| | | | | MKQTREQETPPDFFYFSDYERHNAEIAAF |
| | | | | HLDRILDFRRVPPVAGRMVNMTKEIRDV |
| | | | | TRDKKLWRTFFISPANNICFYGECSYYCS |
| | | | | TEHALCGKPDQIEGSLAAFLPDLSLAKRK |
| | | | | TWRNPWRRSYHKRKKAEWEVDPDYCEE |
| | | | | VKQTPPYDSSHRILDVMDMTIFDFLMGN |
| | | | | MDRHHYETFEKEGNETFIIHLDNGRGFG |
| | | | | KYSHDELSILVPLQQCCRIRKSTYLRLQL |
| | | | | LAKEEYKLSLLMAESLRGDQVAPVLYQP |
| | | | | HLEALDRRLRVVLKAVRDCVERNGLHSV |
| | | | | VDDDLDTEHRAASAR |
| 358 | ScMNN6 | Fam20c | ScMNN6( | MHVLLSKKIARFLLISFVFVLALMVTINH |
| | (M1-P30) | (D93- | M1-P30)_ | PDFSSDPSSNLSSHSLEKLPPAAEPAERAL |
| | | R584) | Fam20c | RGRDPGALRPHDPAHRPLLRDPGPRRSE |
| | | | (D93- | SPPGPGGDASLLARLFEHPLYRVAVPPLT |
| | | | R584) | EEDVLFNVNSDTRLSPKAAENPDWPHAG |
| | | | | AEGAEFLSPGEAAVDSYPNWLKFHIGINR |
| | | | | YELYSRHNPAIEALLHDLSSQRITSVAMK |
| | | | | SGGTQLKLIMTFQNYGQALFKPMKQTR |
| | | | | EQETPPDFFYFSDYERHNAEIAAFHLDRI |
| | | | | LDFRRVPPVAGRMVNMTKEIRDVTRDK |
| | | | | KLWRTFFISPANNICFYGECSYYCSTEHA |
| | | | | LCGKPDQIEGSLAAFLPDLSLAKRKTWR |
| | | | | NPWRRSYHKRKKAEWEVDPDYCEEVKQ |
| | | | | TPPYDSSHRILDVMDMTIFDFLMGNMDR |
| | | | | HHYETFEKFGNETFIHLDNGRGFGKYSH |
| | | | | DELSILVPLQQCCRIRKSTYLRLQLLAKE |
| | | | | EYKLSLLMAESLRGDQVAPVLYQPHLEA |
| | | | | LDRRLRVVLKAVRDCVERNGLHSVVDD |
| | | | | DLDTEHRAASAR |
| 359 | ScMNN6 | Fam20c | ScMINN6( | MHVLLSKKIARFLLISFVFVLALMVTINH |
| | (M1-V85) | (D93- | M1-V85)_ | PKTKQMSEQYVTPYLPKSLQPIAKISAEE |
| | | R584) | Fam20c | QRRIQSEQEEAELKQSLEGEAIRNATVDF |
| | | | (D93- | SSDPSSNLSSHSLEKLPPAAEPAERALRGR |
| | | | R584) | DPGALRPHDPAHRPLLRDPGPRRSESPPG |
| | | | | PGGDASLLARLFEHPLYRVAVPPLTEEDV |
| | | | | LFNVNSDTRLSPKAAENPDWPHAGAEGA |
| | | | | EFLSPGEAAVDSYPNWLKFHIGINRYELY |
| | | | | SRHNPAIEALLHDLSSQRITSVAMKSGGT |
| | | | | QLKLIMTFQNYGQALFKPMKQTREQET |
| | | | | PPDFFYFSDYERHNAEIAAFHLDRILDFR |
| | | | | RVPPVAGRMVNMTKEIRDVTRDKKLWR |
| | | | | TFFISPANNICFYGECSYYCSTEHALCGKP |
| | | | | DQIEGSLAAFLPDLSLAKRKTWRNPWRR |
| | | | | SYHKRKKAEWEVDPDYCEEVKQTPPYDS |
| | | | | SHRILDVMDMTIFDFLMGNMDRHHYETF |
| | | | | EKFGNETFIIHLDNGRGFGKYSHDELSIL |
| | | | | VPLQQCCRIRKSTYLRLQLLAKEEYKLS |
| | | | | LLMAESLRGDQVAPVLYQPHLEALDRRL |
| | | | | RVVLKAVRDCVERNGLHSVVDDDLDTE |
| | | | | HRAASAR |
| 360 | ScMNN6 | Fam20c | ScMNN6 | MHVLLSKKIARFLLISFVFVLALMVTINH |
| | (M1-E160) | (D93- | (M1-E160)_ | PKTKQMSEQYVTPYLPKSLQPIAKISAEE |
| | | R584) | Fam20c | QRRIQSEQEEAELKQSLEGEAIRNATVNA |
| | | | (D93- | IKEKIKSYGGNETTLGFMVPSYINHRGSP |
| | | | R584) | PKACFVSLITERDSMTQILQSIDEVQVKF |
| | | | | NKNFAYPWVFISQGEDFSSDPSSNLSSHSL |
| | | | | EKLPPAAEPAERALRGRDPGALRPHDPA |
| | | | | HRPLLRDPGPRRSESPPGPGGDASLLARL |
| | | | | FEHPLYRVAVPPLTEEDVLFNVNSDTRLS |
| | | | | PKAAENPDWPHAGAEGAEFLSPGEAAVD |
| | | | | SYPNWLKFHIGINRYELYSRHNPAIEALL |
| | | | | HDLSSQRITSVAMKSGGTQLKLIMTFQN |
| | | | | YGQALFKPMKQTREQETPPDFFYFSDYE |
| | | | | RHNAEIAAFHLDRILDFRRVPPVAGRMV |
| | | | | NMTKEIRDVTRDKKLWRTFFISPANNICF |
| | | | | YGECSYYCSTEHALCGKPDQIEGSLAAFL |
| | | | | PDLSLAKRKTWRNPWRRSYHKRKKAEW |
| | | | | EVDPDYCEEVKQTPPYDSSHRILDVMDM |
| | | | | TIFDFLMGNMDRHHYETFEKFGNETFIIH |
| | | | | LDNGRGFGKYSHDELSILVPLQQCCRIRK |
| | | | | STYLRLQLLAKEEYKLSLLMAESLRGDQ |
| | | | | VAPVLYQPHLEALDRRLRVVLKAVRDCV |
| | | | | ERNGLHSVVDDDLDTEHRAASAR |

TABLE 11
Polynucleic acid sequences encoding the engineered kinases

| SEQ | | |
|---|---|---|
| ID | | |
| NO: | Construct Name | Polynucleic acid sequence |
| 361 | Fam20c(M1- | ATGAAAATGATGTTGGTTAGAAGATTTAGAGTTTTGAT |
| | R584)_FLAG | TTTGATGGTTTTTTTGGTTGCTTGTGCTTTGCATATTGC |
| | | TTTGGATTTGTTGCCAAGATTGGAAAGAAGAGGTGCTA |
| | | GACCATCTGGTGAACCTGGTTGTTCTTGTGCTCAACCT |
| | | GCTGCTGAAGTCGCTGCTCCTGGTTGGGCTCAAGTTAG |
| | | AGGTAGACCTGGTGAACCACCTGCTGCTTCGTCAGCTG |
| | | CTGGTGATGCTGGTTGGCCAAATAAACATACTTTGAGA |
| | | ATTTTGCAAGATTTTTCTTCTGATCCATCTTCTAATTTA |
| | | TCTAGTCACTCTTTGGAAAAATTGCCACCTGCTGCTGA |
| | | GCCTGCTGAAAGAGCTTTGAGAGGTAGAGACCCTGGT |
| | | GCGCTCAGACCACATGATCCTGCTCATAGACCATTGTT |
| | | GAGAGACCCTGGTCCTAGAAGATCTGAATCTCCACCTG |
| | | GTCCTGGTGGTGATGCTTCTTTGTTGGCTAGATTGTTT |
| | | GAACATCCATTGTATAGAGTTGCTGTTCCACCATTGAC |
| | | TGAAGAAGATGTTTTGTTTAATGTTAATTCTGATACTA |
| | | GATTGTCTCCAAAAGCTGCTGAAAATCCTGATTGGCCA |
| | | CATGCTGGTGCTGAAGGTGCTGAATTTTTGTCTCCTGG |
| | | TGAAGCGGCTGTTGATTCCTATCCAAATTGGTTGAAAT |
| | | TCCACATCGGTATTAACCGATACGAATTGTATTCTAGA |
| | | CATAATCCTGCTATTGAAGCTTTGTTGCATGATTTGTCT |
| | | AGTCAGAGAATTACTTCTGTTGCTATGAAGTCTGGTGG |
| | | TACTCAGTTGAAGTTGATAATGACTTTTCAAAACTATG |
| | | GGCAAGCTTTGTTTAAACCAATGAAACAAACGAGAGAA |
| | | CAAGAAACTCCACCTGATTTTTTTTATTTTTCGGATTAT |
| | | GAAAGACATAATGCTGAAATTGCTGCTTTCCACTTGGA |
| | | CAGAATATTGGATTTTCGCAGAGTTCCACCTGTTGCTG |
| | | GTCGGATGGTTAACATGACTAAGGAAATTAGAGATGTT |
| | | ACTAGAGATAAAAAATTGTGGAGAACTTTTTTCATTTC |
| | | TCCGGCTAATAATATTTGTTTTTACGGGGAATGTTCTT |
| | | ATTATTGTTCTACTGAACATGCTTTGTGTGGTAAACCT |
| | | GATCAAATTGAAGGTTCTTTGGCTGCTTTTTTGCCTGA |
| | | TTTGTCTTTGGCTAAAAGAAAAACTTGGAGAAATCCAT |
| | | GGAGAAGATCTTATCATAAAAGAAAAAAAGCTGAATGG |
| | | GAAGTTGATCCTGATTATTGTGAAGAAGTTAAACAAAC |
| | | TCCACCATATGATTCGTCTCATAGAATATTGGACGTCA |
| | | TGGATATGACGATCTTTGACTTTCTGATGGGGAACATG |
| | | GACAGACATCACTATGAAACATTCGAAAAATTCGGTAA |
| | | TGAAACTTTTATCATCCATTTGGATAATGGTAGAGGTT |
| | | TTGGTAAATATTCTCATGATGAATTGTCTATTTTGGTTC |
| | | CATTGCAACAGTGTTGTAGAATAAGGAAAAGCACTTAC |
| | | TTAAGATTACAACTCTTGGCTAAAGAAGAATATAAATT |
| | | GTCTTTGTTGATGGCTGAATCTTTGAGAGGTGATCAAG |
| | | TTGCTCCTGTTTTGTATCAACCACATTTGGAAGCTTTG |
| | | GATAGAAGATTGAGAGTTGTTTTGAAAGCTGTTAGAGA |
| | | TTGTGTTGAAAGAAATGGTTTGCATTCTGTTGTTGATG |
| | | ATGATTTGGATACTGAACATAGAGCTGCTTCTGCTAGA |
| | | GATTATAAAGATGATGATGATAAATAA |
| 362 | Fam20c(R32- | ATGAGATTGGAAAGAAGAGGTGCTAGACCATCTGGTG |
| | R584)_FLAG | AACCTGGTTGTTCTTGTGCTCAACCTGCTGCTGAAGTC |
| | | GCTGCTCCTGGTTGGGCTCAAGTTAGAGGTAGACCTG |
| | | GTGAACCACCTGCTGCTTCGTCAGCTGCTGGTGATGCT |
| | | GGTTGGCCAAATAAACATACTTTGAGAATTTTGCAAGA |
| | | TTTTTCTTCTGATCCATCTTCTAATTTATCTAGTCACTC |
| | | TTTGGAAAAATTGCCACCTGCTGCTGAGCCTGCTGAAA |
| | | GAGCTTTGAGAGGTAGAGACCCTGGTGCGCTCAGACC |
| | | ACATGATCCTGCTCATAGACCATTGTTGAGAGACCCTG |
| | | GTCCTAGAAGATCTGAATCTCCACCTGGTCCTGGTGGT |
| | | GATGCTTCTTTGTTGGCTAGATTGTTTGAACATCCATT |
| | | GTATAGAGTTGCTGTTCCACCATTGACTGAAGAAGATG |
| | | TTTTGTTTAATGTTAATTCTGATACTAGATTGTCTCCAA |
| | | AAGCTGCTGAAAATCCTGATTGGCCACATGCTGGTGCT |
| | | GAAGGTGCTGAATTTTTGTCTCCTGGTGAAGCGGCTGT |
| | | TGATTCCTATCCAAATTGGTTGAAATTCCACATCGGTA |
| | | TTAACCGATACGAATTGTATTCTAGACATAATCCTGCT |
| | | ATTGAAGCTTTGTTGCATGATTTGTCTAGTCAGAGAAT |
| | | TACTTCTGTTGCTATGAAGTCTGGTGGTACTCAGTTGA |
| | | AGTTGATAATGACTTTTCAAAACTATGGGCAAGCTTTG |
| | | TTTAAACCAATGAAACAAACGAGAGAACAAGAAACTCC |
| | | ACCTGATTTTTTTTATTTTTCGGATTATGAAAGACATAA |
| | | TGCTGAAATTGCTGCTTTCCACTTGGACAGAATATTGG |
| | | ATTTTCGCAGAGTTCCACCTGTTGCTGGTCGGATGGTT |
| | | AACATGACTAAGGAAATTAGAGATGTTACTAGAGATAA |
| | | AAAATTGTGGAGAACTTTTTTCATTTCTCCGGCTAATA |
| | | ATATTTGTTTTTACGGGGAATGTTCTTATTATTGTTCTA |
| | | CTGAACATGCTTTGTGTGGTAAACCTGATCAAATTGAA |
| | | GGTTCTTTGGCTGCTTTTTTGCCTGATTTGTCTTTGGCT |
| | | AAAAGAAAAACTTGGAGAAATCCATGGAGAAGATCTTA |
| | | TCATAAAAGAAAAAAAGCTGAATGGGAAGTTGATCCTG |
| | | ATTATTGTGAAGAAGTTAAACAAACTCCACCATATGAT |
| | | TCGTCTCATAGAATATTGGACGTCATGGATATGACGAT |
| | | CTTTGACTTTCTGATGGGGAACATGGACAGACATCACT |
| | | ATGAAACATTCGAAAAATTCGGTAATGAAACTTTTATC |
| | | ATCCATTTGGATAATGGTAGAGGTTTTGGTAAATATTC |
| | | TCATGATGAATTGTCTATTTTGGTTCCATTGCAACAGT |
| | | GTTGTAGAATAAGGAAAAGCACTTACTTAAGATTACAA |
| | | CTCTTGGCTAAAGAAGAATATAAATTGTCTTTGTTGAT |
| | | GGCTGAATCTTTGAGAGGTGATCAAGTTGCTCCTGTTT |
| | | TGTATCAACCACATTTGGAAGCTTTGGATAGAAGATTG |
| | | AGAGTTGTTTTGAAAGCTGTTAGAGATTGTGTTGAAAG |
| | | AAATGGTTTGCATTCTGTTGTTGATGATGATTTGGATA |
| | | CTGAACATAGAGCTGCTTCTGCTAGAGATTATAAAGAT |
| | | GATGATGATAAATAA |
| 363 | Fam20c(R64- | AGAGGTAGACCTGGTGAACCACCGGCTGCGTCCTCTG |
| | R584)_FLAG | CTGCTGGTGATGCTGGTTGGCCAAATAAACATACTTTG |
| | | AGAATTTTGCAAGATTTTTCTTCTGATCCATCTTCTAAT |
| | | CTATCTAGTCACTCATTGGAAAAATTGCCACCTGCTGC |
| | | AGAGCCTGCTGAAAGAGCTTTGAGAGGTAGAGATCCC |
| | | GGTGCGTTAAGACCACATGATCCTGCTCATAGACCATT |
| | | GTTGAGAGATCCTGGTCCAAGAAGATCTGAATCTCCAC |
| | | CTGGTCCTGGTGGTGATGCTTCTTTGTTGGCTAGATTG |
| | | TTTGAACATCCATTGTATAGAGTTGCTGTTCCACCATT |
| | | GACTGAAGAAGATGTTTTGTTTAATGTTAATTCTGATA |
| | | CTAGATTGTCTCCAAAAGCTGCTGAAAATCCTGATTGG |
| | | CCACATGCTGGTGCTGAAGGTGCTGAATTTTTGTCTCC |
| | | TGGTGAAGCAGCTGTTGATTCTTATCCCAATTGGTTGA |
| | | AATTCCATATTGGTATTAACAGATACGAATTGTATTCTA |
| | | GACATAATCCTGCTATTGAAGCTTTGTTGCATGATCTT |
| | | TCTTCTCAACGTATTACTTCTGTTGCTATGAAATCGGG |
| | | TGGTACTCAATTGAAATTGATTATGACTTTTCAAAACTA |
| | | TGGTCAAGCTTTGTTTAAACCAATGAAACAGACTAGAG |
| | | AGCAAGAAACGCCACCTGATTTTTTTTACTTCTCTGATT |
| | | ATGAAAGACATAATGCTGAAATTGCTGCTTTTCACTTG |
| | | GACCGTATATTGGATTTTCGCAGAGTTCCACCTGTTGC |
| | | TGGTCGTATGGTTAACATGACTAAGGAAATTAGAGATG |
| | | TTACTCGCGACAAAAAATTGTGGAGAACTTTTTTTATA |
| | | TCTCCGGCCAATAATATTTGTTTTTATGGTGAATGTTCT |
| | | TATTATTGTTCTACTGAACATGCTTTGTGTGGTAAACCT |
| | | GATCAAATTGAAGGTTCTTTGGCTGCTTTTTTGCCTGA |
| | | TTTGTCTTTGGCTAAAAGAAAAACTTGGAGAAATCCAT |
| | | GGAGAAGATCTTATCATAAAAGAAAAAAAGCTGAATGG |
| | | GAAGTTGATCCTGATTATTGTGAAGAAGTTAAACAAAC |
| | | TCCACCATATGATTCTTCTCACAGAATTTTGGATGTTAT |
| | | GGATATGACTATTTTCGATTTTTTGATGGGTAACATGG |
| | | ATAGACATCATTATGAAACGTTTGAGAAATTTGGGAAT |
| | | GAAACTTTTATTATTCATTTGGACAACGGTAGAGGTTT |
| | | TGGTAAATATTCTCATGATGAATTGTCTATTTTGGTTCC |
| | | ATTGCAGCAATGTTGCAGAATAAGAAAATCGACTTATT |
| | | TGCGACTGCAGCTTTTGGCTAAAGAAGAATATAAATTG |
| | | TCTTTGTTGATGGCTGAATCTTTGAGAGGTGATCAAGT |
| | | TGCTCCTGTTTTGTATCAACCACATTTGGAAGCTTTGG |
| | | ATAGAAGATTGAGAGTTGTTTTGAAAGCTGTTAGAGAT |
| | | TGTGTTGAAAGAAATGGTTTGCATTCTGTTGTTGATGA |
| | | TGATTTGGATACTGAACATAGAGCTGCTTCTGCTAGAG |
| | | ATTATAAAGATGATGATGATAAATAA |
| 364 | Fam20c(D93- | GATTTTTCTTCTGATCCATCTTCTAATCTATCTAGTCAC |
| | R584)_FLAG | TCATTGGAAAAATTGCCACCTGCTGCAGAGCCTGCTGA |
| | | AAGAGCTTTGAGAGGTAGAGATCCCGGTGCGTTAAGA |
| | | CCACATGATCCTGCTCATAGACCATTGTTGAGAGATCC |
| | | TGGTCCAAGAAGATCTGAATCTCCACCTGGTCCTGGTG |
| | | GTGATGCTTCTTTGTTGGCTAGATTGTTTGAACATCCA |
| | | TTGTATAGAGTTGCTGTTCCACCATTGACTGAAGAAGA |
| | | TGTTTTGTTTAATGTTAATTCTGATACTAGATTGTCTCC |
| | | AAAAGCTGCTGAAAATCCTGATTGGCCACATGCTGGTG |
| | | CTGAAGGTGCTGAATTTTTGTCTCCTGGTGAAGCAGCT |
| | | GTTGATTCTTATCCCAATTGGTTGAAATTCCATATTGGT |
| | | ATTAACAGATACGAATTGTATTCTAGACATAATCCTGC |
| | | TATTGAAGCTTTGTTGCATGATCTTTCTTCTCAACGTAT |
| | | TACTTCTGTTGCTATGAAATCGGGTGGTACTCAATTGA |
| | | AATTGATTATGACTTTTCAAAACTATGGTCAAGCTTTGT |
| | | TTAAACCAATGAAACAGACTAGAGAGCAAGAAACGCCA |
| | | CCTGATTTTTTTTACTTCTCTGATTATGAAAGACATAAT |
| | | GCTGAAATTGCTGCTTTTCACTTGGACCGTATATTGGA |
| | | TTTTCGCAGAGTTCCACCTGTTGCTGGTCGTATGGTTA |
| | | ACATGACTAAGGAAATTAGAGATGTTACTCGCGACAAA |
| | | AAATTGTGGAGAACTTTTTTTATATCTCCGGCCAATAA |
| | | TATTTGTTTTTATGGTGAATGTTCTTATTATTGTTCTAC |
| | | TGAACATGCTTTGTGTGGTAAACCTGATCAAATTGAAG |
| | | GTTCTTTGGCTGCTTTTTTGCCTGATTTGTCTTTGGCTA |
| | | AAAGAAAAACTTGGAGAAATCCATGGAGAAGATCTTAT |
| | | CATAAAAGAAAAAAAGCTGAATGGGAAGTTGATCCTGA |
| | | TTATTGTGAAGAAGTTAAACAAACTCCACCATATGATT |
| | | CTTCTCACAGAATTTTGGATGTTATGGATATGACTATTT |
| | | TCGATTTTTTGATGGGTAACATGGATAGACATCATTAT |
| | | GAAACGTTTGAGAAATTTGGGAATGAAACTTTTATTAT |
| | | TCATTTGGACAACGGTAGAGGTTTTGGTAAATATTCTC |
| | | ATGATGAATTGTCTATTTTGGTTCCATTGCAGCAATGT |
| | | TGCAGAATAAGAAAATCGACTTATTTGCGACTGCAGCT |
| | | TTTGGCTAAAGAAGAATATAAATTGTCTTTGTTGATGG |
| | | CTGAATCTTTGAGAGGTGATCAAGTTGCTCCTGTTTTG |
| | | TATCAACCACATTTGGAAGCTTTGGATAGAAGATTGAG |
| | | AGTTGTTTTGAAAGCTGTTAGAGATTGTGTTGAAAGAA |
| | | ATGGTTTGCATTCTGTTGTTGATGATGATTTGGATACT |
| | | GAACATAGAGCTGCTTCTGCTAGAGATTATAAAGATGA |
| | | TGATGATAAATAA |
| 365 | Fam20c(Q289- | CAGACTAGAGAGCAAGAAACGCCACCTGATTTTTTTTA |
| | R584)_FLAG | CTTCTCTGATTATGAAAGACATAATGCTGAAATTGCTG |
| | | CTTTTCACTTGGACCGTATATTGGATTTTCGCAGAGTT |
| | | CCACCTGTTGCTGGTCGTATGGTTAACATGACTAAGGA |
| | | AATTAGAGATGTTACTCGCGACAAAAAATTGTGGAGAA |
| | | CTTTTTTTATATCTCCGGCCAATAATATTTGTTTTTATG |
| | | GTGAATGTTCTTATTATTGTTCTACTGAACATGCTTTGT |
| | | GTGGTAAACCTGATCAAATTGAAGGTTCTTTGGCTGCT |
| | | TTTTTGCCTGATTTGTCTTTGGCTAAAAGAAAAACTTG |
| | | GAGAAATCCATGGAGAAGATCTTATCATAAAAGAAAAA |
| | | AAGCTGAATGGGAAGTTGATCCTGATTATTGTGAAGAA |
| | | GTTAAACAAACTCCACCATATGATTCTTCTCACAGAAT |
| | | TTTGGATGTTATGGATATGACTATTTTCGATTTTTTGAT |
| | | GGGTAACATGGATAGACATCATTATGAAACGTTTGAGA |
| | | AATTTGGGAATGAAACTTTTATTATTCATTTGGACAAC |
| | | GGTAGAGGTTTTGGTAAATATTCTCATGATGAATTGTC |
| | | TATTTTGGTTCCATTGCAGCAATGTTGCAGAATAAGAA |
| | | AATCGACTTATTTGCGACTGCAGCTTTTGGCTAAAGAA |
| | | GAATATAAATTGTCTTTGTTGATGGCTGAATCTTTGAG |
| | | AGGTGATCAAGTTGCTCCTGTTTTGTATCAACCACATT |
| | | TGGAAGCTTTGGATAGAAGATTGAGAGTTGTTTTGAAA |
| | | GCTGTTAGAGATTGTGTTGAAAGAAATGGTTTGCATTC |
| | | TGTTGTTGATGATGATTTGGATACTGAACATAGAGCTG |
| | | CTTCTGCTAGAGATTATAAAGATGATGATGATAAATAA |
| 366 | Fam20c(M1- | ATGAAAATGATGTTGGTTAGAAGATTTAGAGTTTTGAT |
| | R584)_MYC | TTTGATGGTTTTTTTGGTTGCTTGTGCTTTGCATATTGC |
| | | TTTGGATTTGTTGCCAAGATTGGAAAGAAGAGGTGCTA |
| | | GACCATCTGGTGAACCTGGTTGTTCTTGTGCTCAACCT |
| | | GCTGCTGAAGTCGCTGCTCCTGGTTGGGCTCAAGTTAG |
| | | AGGTAGACCTGGTGAACCACCTGCTGCTTCGTCAGCTG |
| | | CTGGTGATGCTGGTTGGCCAAATAAACATACTTTGAGA |
| | | ATTTTGCAAGATTTTTCTTCTGATCCATCTTCTAATTTA |
| | | TCTAGTCACTCTTTGGAAAAATTGCCACCTGCTGCTGA |
| | | GCCTGCTGAAAGAGCTTTGAGAGGTAGAGACCCTGGT |
| | | GCGCTCAGACCACATGATCCTGCTCATAGACCATTGTT |
| | | GAGAGACCCTGGTCCTAGAAGATCTGAATCTCCACCTG |
| | | GTCCTGGTGGTGATGCTTCTTTGTTGGCTAGATTGTTT |
| | | GAACATCCATTGTATAGAGTTGCTGTTCCACCATTGAC |
| | | TGAAGAAGATGTTTTGTTTAATGTTAATTCTGATACTA |
| | | GATTGTCTCCAAAAGCTGCTGAAAATCCTGATTGGCCA |
| | | CATGCTGGTGCTGAAGGTGCTGAATTTTTGTCTCCTGG |
| | | TGAAGCGGCTGTTGATTCCTATCCAAATTGGTTGAAAT |
| | | TCCACATCGGTATTAACCGATACGAATTGTATTCTAGA |
| | | CATAATCCTGCTATTGAAGCTTTGTTGCATGATTTGTCT |
| | | AGTCAGAGAATTACTTCTGTTGCTATGAAGTCTGGTGG |
| | | TACTCAGTTGAAGTTGATAATGACTTTTCAAAACTATG |
| | | GGCAAGCTTTGTTTAAACCAATGAAACAAACGAGAGAA |
| | | CAAGAAACTCCACCTGATTTTTTTTATTTTTCGGATTAT |
| | | GAAAGACATAATGCTGAAATTGCTGCTTTCCACTTGGA |
| | | CAGAATATTGGATTTTCGCAGAGTTCCACCTGTTGCTG |
| | | GTCGGATGGTTAACATGACTAAGGAAATTAGAGATGTT |
| | | ACTAGAGATAAAAAATTGTGGAGAACTTTTTTCATTTC |
| | | TCCGGCTAATAATATTTGTTTTTACGGGGAATGTTCTT |
| | | ATTATTGTTCTACTGAACATGCTTTGTGTGGTAAACCT |
| | | GATCAAATTGAAGGTTCTTTGGCTGCTTTTTTGCCTGA |
| | | TTTGTCTTTGGCTAAAAGAAAAACTTGGAGAAATCCAT |
| | | GGAGAAGATCTTATCATAAAAGAAAAAAAGCTGAATGG |
| | | GAAGTTGATCCTGATTATTGTGAAGAAGTTAAACAAAC |
| | | TCCACCATATGATTCGTCTCATAGAATATTGGACGTCA |
| | | TGGATATGACGATCTTTGACTTTCTGATGGGGAACATG |
| | | GACAGACATCACTATGAAACATTCGAAAAATTCGGTAA |
| | | TGAAACTTTTATCATCCATTTGGATAATGGTAGAGGTT |
| | | TTGGTAAATATTCTCATGATGAATTGTCTATTTTGGTTC |
| | | CATTGCAACAGTGTTGTAGAATAAGGAAAAGCACTTAC |
| | | TTAAGATTACAACTCTTGGCTAAAGAAGAATATAAATT |
| | | GTCTTTGTTGATGGCTGAATCTTTGAGAGGTGATCAAG |
| | | TTGCTCCTGTTTTGTATCAACCACATTTGGAAGCTTTG |
| | | GATAGAAGATTGAGAGTTGTTTTGAAAGCTGTTAGAGA |
| | | TTGTGTTGAAAGAAATGGTTTGCATTCTGTTGTTGATG |
| | | ATGATTTGGATACTGAACATAGAGCTGCTTCTGCTAGA |
| | | GAACAGAAGCTGATCAGTGAAGAAGATCTGTGATGA |
| 367 | Fam20c(R32- | ATGAGATTGGAAAGAAGAGGTGCTAGACCATCTGGTG |
| | R584)_FLAG | AACCTGGTTGTTCTTGTGCTCAACCTGCTGCTGAAGTC |
| | | GCTGCTCCTGGTTGGGCTCAAGTTAGAGGTAGACCTG |
| | | GTGAACCACCTGCTGCTTCGTCAGCTGCTGGTGATGCT |
| | | GGTTGGCCAAATAAACATACTTTGAGAATTTTGCAAGA |
| | | TTTTTCTTCTGATCCATCTTCTAATTTATCTAGTCACTC |
| | | TTTGGAAAAATTGCCACCTGCTGCTGAGCCTGCTGAAA |
| | | GAGCTTTGAGAGGTAGAGACCCTGGTGCGCTCAGACC |
| | | ACATGATCCTGCTCATAGACCATTGTTGAGAGACCCTG |
| | | GTCCTAGAAGATCTGAATCTCCACCTGGTCCTGGTGGT |
| | | GATGCTTCTTTGTTGGCTAGATTGTTTGAACATCCATT |
| | | GTATAGAGTTGCTGTTCCACCATTGACTGAAGAAGATG |
| | | TTTTGTTTAATGTTAATTCTGATACTAGATTGTCTCCAA |
| | | AAGCTGCTGAAAATCCTGATTGGCCACATGCTGGTGCT |
| | | GAAGGTGCTGAATTTTTGTCTCCTGGTGAAGCGGCTGT |
| | | TGATTCCTATCCAAATTGGTTGAAATTCCACATCGGTA |
| | | TTAACCGATACGAATTGTATTCTAGACATAATCCTGCT |
| | | ATTGAAGCTTTGTTGCATGATTTGTCTAGTCAGAGAAT |
| | | TACTTCTGTTGCTATGAAGTCTGGTGGTACTCAGTTGA |
| | | AGTTGATAATGACTTTTCAAAACTATGGGCAAGCTTTG |
| | | TTTAAACCAATGAAACAAACGAGAGAACAAGAAACTCC |
| | | ACCTGATTTTTTTTATTTTTCGGATTATGAAAGACATAA |
| | | TGCTGAAATTGCTGCTTTCCACTTGGACAGAATATTGG |
| | | ATTTTCGCAGAGTTCCACCTGTTGCTGGTCGGATGGTT |
| | | AACATGACTAAGGAAATTAGAGATGTTACTAGAGATAA |
| | | AAAATTGTGGAGAACTTTTTTCATTTCTCCGGCTAATA |
| | | ATATTTGTTTTTACGGGGAATGTTCTTATTATTGTTCTA |
| | | CTGAACATGCTTTGTGTGGTAAACCTGATCAAATTGAA |
| | | GGTTCTTTGGCTGCTTTTTTGCCTGATTTGTCTTTGGCT |
| | | AAAAGAAAAACTTGGAGAAATCCATGGAGAAGATCTTA |
| | | TCATAAAAGAAAAAAAGCTGAATGGGAAGTTGATCCTG |
| | | ATTATTGTGAAGAAGTTAAACAAACTCCACCATATGAT |
| | | TCGTCTCATAGAATATTGGACGTCATGGATATGACGAT |
| | | CTTTGACTTTCTGATGGGGAACATGGACAGACATCACT |
| | | ATGAAACATTCGAAAAATTCGGTAATGAAACTTTTATC |
| | | ATCCATTTGGATAATGGTAGAGGTTTTGGTAAATATTC |
| | | TCATGATGAATTGTCTATTTTGGTTCCATTGCAACAGT |
| | | GTTGTAGAATAAGGAAAAGCACTTACTTAAGATTACAA |
| | | CTCTTGGCTAAAGAAGAATATAAATTGTCTTTGTTGAT |
| | | GGCTGAATCTTTGAGAGGTGATCAAGTTGCTCCTGTTT |
| | | TGTATCAACCACATTTGGAAGCTTTGGATAGAAGATTG |
| | | AGAGTTGTTTTGAAAGCTGTTAGAGATTGTGTTGAAAG |
| | | AAATGGTTTGCATTCTGTTGTTGATGATGATTTGGATA |
| | | CTGAACATAGAGCTGCTTCTGCTAGAGATTATAAAGAT |
| | | GATGATGATAAATGA |
| 368 | ScKRE(M1- | ATGGCTTTATTTTTGTCTAAAAGATTGTTGAGATTTACT |
| | 158)_Fam20c(R3 | GTTATTGCTGGTGCTGTTATTGTTCTATTGTTGACGCT |
| | 2-R584)_FLAG | CAATTCAAATTCACGAACTCAACAATATATTCCATCTTC |
| | | TATTTCTGCTGCTTTTGATTTTACTTCTGGTTCTATTTC |
| | | TCCTGAACAACAAGTTATTAGATTGGAAAGAAGAGGTG |
| | | CTAGACCATCTGGTGAACCTGGTTGTTCTTGTGCTCAA |
| | | CCTGCTGCTGAAGTCGCTGCTCCTGGTTGGGCTCAAGT |
| | | TAGAGGTAGACCTGGTGAACCACCTGCTGCTTCGTCAG |
| | | CTGCTGGTGATGCTGGTTGGCCAAATAAACATACTTTG |
| | | AGAATTTTGCAAGATTTTTCTTCTGATCCATCTTCTAAT |
| | | TTATCTAGTCACTCTTTGGAAAAATTGCCACCTGCTGC |
| | | TGAGCCTGCTGAAAGAGCTTTGAGAGGTAGAGACCCT |
| | | GGTGCGCTCAGACCACATGATCCTGCTCATAGACCATT |
| | | GTTGAGAGACCCTGGTCCTAGAAGATCTGAATCTCCAC |
| | | CTGGTCCTGGTGGTGATGCTTCTTTGTTGGCTAGATTG |
| | | TTTGAACATCCATTGTATAGAGTTGCTGTTCCACCATT |
| | | GACTGAAGAAGATGTTTTGTTTAATGTTAATTCTGATA |
| | | CTAGATTGTCTCCAAAAGCTGCTGAAAATCCTGATTGG |
| | | CCACATGCTGGTGCTGAAGGTGCTGAATTTTTGTCTCC |
| | | TGGTGAAGCGGCTGTTGATTCCTATCCAAATTGGTTGA |
| | | AATTCCACATCGGTATTAACCGATACGAATTGTATTCT |
| | | AGACATAATCCTGCTATTGAAGCTTTGTTGCATGATTT |
| | | GTCTAGTCAGAGAATTACTTCTGTTGCTATGAAGTCTG |
| | | GTGGTACTCAGTTGAAGTTGATAATGACTTTTCAAAAC |
| | | TATGGGCAAGCTTTGTTTAAACCAATGAAACAAACGAG |
| | | AGAACAAGAAACTCCACCTGATTTTTTTTATTTTTCGGA |
| | | TTATGAAAGACATAATGCTGAAATTGCTGCTTTCCACT |
| | | TGGACAGAATATTGGATTTTCGCAGAGTTCCACCTGTT |
| | | GCTGGTCGGATGGTTAACATGACTAAGGAAATTAGAGA |
| | | TGTTACTAGAGATAAAAAATTGTGGAGAACTTTTTTCA |
| | | TTTCTCCGGCTAATAATATTTGTTTTTACGGGGAATGTT |
| | | CTTATTATTGTTCTACTGAACATGCTTTGTGTGGTAAAC |
| | | CTGATCAAATTGAAGGTTCTTTGGCTGCTTTTTTGCCT |
| | | GATTTGTCTTTGGCTAAAAGAAAAACTTGGAGAAATCC |
| | | ATGGAGAAGATCTTATCATAAAAGAAAAAAAGCTGAAT |
| | | GGGAAGTTGATCCTGATTATTGTGAAGAAGTTAAACAA |
| | | ACTCCACCATATGATTCGTCTCATAGAATATTGGACGT |
| | | CATGGATATGACGATCTTTGACTTTCTGATGGGGAACA |
| | | TGGACAGACATCACTATGAAACATTCGAAAAATTCGGT |
| | | AATGAAACTTTTATCATCCATTTGGATAATGGTAGAGG |
| | | TTTTGGTAAATATTCTCATGATGAATTGTCTATTTTGGT |
| | | TCCATTGCAACAGTGTTGTAGAATAAGGAAAAGCACTT |
| | | ACTTAAGATTACAACTCTTGGCTAAAGAAGAATATAAA |
| | | TTGTCTTTGTTGATGGCTGAATCTTTGAGAGGTGATCA |
| | | AGTTGCTCCTGTTTTGTATCAACCACATTTGGAAGCTT |
| | | TGGATAGAAGATTGAGAGTTGTTTTGAAAGCTGTTAGA |
| | | GATTGTGTTGAAAGAAATGGTTTGCATTCTGTTGTTGA |
| | | TGATGATTTGGATACTGAACATAGAGCTGCTTCTGCTA |
| | | GAGATTATAAAGATGATGATGATAAATGA |
| 369 | ScKRE2(M1- | ATGGCTTTATTTTTGTCTAAAAGATTGTTGAGATTTACT |
| | S80)_Fam20c(R3 | GTTATTGCTGGTGCTGTTATTGTTCTATTGTTGACGCT |
| | 2-R584)_FLAG | CAATTCAAATTCACGAACTCAACAATATATTCCATCTTC |
| | | TATTTCTGCTGCTTTTGATTTTACTTCTGGTTCTATTTC |
| | | TCCTGAACAACAAGTTATTTCTGAAGAAAATGATGCTA |
| | | AGAAATTAGAACAGTCCGCTTTGAATTCTGAAGCTTCT |
| | | GAAGATTCTAGATTGGAAAGAAGAGGTGCTAGACCATC |
| | | TGGTGAACCTGGTTGTTCTTGTGCTCAACCTGCTGCTG |
| | | AAGTCGCTGCTCCTGGTTGGGCTCAAGTTAGAGGTAGA |
| | | CCTGGTGAACCACCTGCTGCTTCGTCAGCTGCTGGTGA |
| | | TGCTGGTTGGCCAAATAAACATACTTTGAGAATTTTGC |
| | | AAGATTTTTCTTCTGATCCATCTTCTAATTTATCTAGTC |
| | | ACTCTTTGGAAAAATTGCCACCTGCTGCTGAGCCTGCT |
| | | GAAAGAGCTTTGAGAGGTAGAGACCCTGGTGCGCTCA |
| | | GACCACATGATCCTGCTCATAGACCATTGTTGAGAGAC |
| | | CCTGGTCCTAGAAGATCTGAATCTCCACCTGGTCCTGG |
| | | TGGTGATGCTTCTTTGTTGGCTAGATTGTTTGAACATC |
| | | CATTGTATAGAGTTGCTGTTCCACCATTGACTGAAGAA |
| | | GATGTTTTGTTTAATGTTAATTCTGATACTAGATTGTCT |
| | | CCAAAAGCTGCTGAAAATCCTGATTGGCCACATGCTGG |
| | | TGCTGAAGGTGCTGAATTTTTGTCTCCTGGTGAAGCGG |
| | | CTGTTGATTCCTATCCAAATTGGTTGAAATTCCACATC |
| | | GGTATTAACCGATACGAATTGTATTCTAGACATAATCC |
| | | TGCTATTGAAGCTTTGTTGCATGATTTGTCTAGTCAGA |
| | | GAATTACTTCTGTTGCTATGAAGTCTGGTGGTACTCAG |
| | | TTGAAGTTGATAATGACTTTTCAAAACTATGGGCAAGC |
| | | TTTGTTTAAACCAATGAAACAAACGAGAGAACAAGAAA |
| | | CTCCACCTGATTTTTTTTATTTTTCGGATTATGAAAGAC |
| | | ATAATGCTGAAATTGCTGCTTTCCACTTGGACAGAATA |
| | | TTGGATTTTCGCAGAGTTCCACCTGTTGCTGGTCGGAT |
| | | GGTTAACATGACTAAGGAAATTAGAGATGTTACTAGAG |
| | | ATAAAAAATTGTGGAGAACTTTTTTCATTTCTCCGGCT |
| | | AATAATATTTGTTTTTACGGGGAATGTTCTTATTATTGT |
| | | TCTACTGAACATGCTTTGTGTGGTAAACCTGATCAAAT |
| | | TGAAGGTTCTTTGGCTGCTTTTTTGCCTGATTTGTCTTT |
| | | GGCTAAAAGAAAAACTTGGAGAAATCCATGGAGAAGA |
| | | TCTTATCATAAAAGAAAAAAAGCTGAATGGGAAGTTGA |
| | | TCCTGATTATTGTGAAGAAGTTAAACAAACTCCACCAT |
| | | ATGATTCGTCTCATAGAATATTGGACGTCATGGATATG |
| | | ACGATCTTTGACTTTCTGATGGGGAACATGGACAGACA |
| | | TCACTATGAAACATTCGAAAAATTCGGTAATGAAACTT |
| | | TTATCATCCATTTGGATAATGGTAGAGGTTTTGGTAAA |
| | | TATTCTCATGATGAATTGTCTATTTTGGTTCCATTGCAA |
| | | CAGTGTTGTAGAATAAGGAAAAGCACTTACTTAAGATT |
| | | ACAACTCTTGGCTAAAGAAGAATATAAATTGTCTTTGT |
| | | TGATGGCTGAATCTTTGAGAGGTGATCAAGTTGCTCCT |
| | | GTTTTGTATCAACCACATTTGGAAGCTTTGGATAGAAG |
| | | ATTGAGAGTTGTTTTGAAAGCTGTTAGAGATTGTGTTG |
| | | AAAGAAATGGTTTGCATTCTGTTGTTGATGATGATTTG |
| | | GATACTGAACATAGAGCTGCTTCTGCTAGAGATTATAA |
| | | AGATGATGATGATAAATGA |
| 370 | ScKRE2(M1- | ATGGCTTTATTTTTGTCTAAAAGATTGTTGAGATTTACT |
| | D102)_ | GTTATTGCTGGTGCTGTTATTGTTCTATTGTTGACGCT |
| | Fam20c(R32- | CAATTCAAATTCACGAACTCAACAATATATTCCATCTTC |
| | R584)_ FLAG | TATTTCTGCTGCTTTTGATTTTACTTCTGGTTCTATTTC |
| | | TCCTGAACAACAAGTTATTTCTGAAGAAAATGATGCTA |
| | | AGAAATTAGAACAGTCCGCTTTGAATTCTGAAGCTTCT |
| | | GAAGATTCTGAAGCTATGGATGAAGAATCTAAAGCTTT |
| | | GAAAGCTGCTGCTGAAAAAGCTGATGCTCCAATTGATA |
| | | GATTGGAAAGAAGAGGTGCTAGACCATCTGGTGAACC |
| | | TGGTTGTTCTTGTGCTCAACCTGCTGCTGAAGTCGCTG |
| | | CTCCTGGTTGGGCTCAAGTTAGAGGTAGACCTGGTGAA |
| | | CCACCTGCTGCTTCGTCAGCTGCTGGTGATGCTGGTTG |
| | | GCCAAATAAACATACTTTGAGAATTTTGCAAGATTTTT |
| | | CTTCTGATCCATCTTCTAATTTATCTAGTCACTCTTTGG |
| | | AAAAATTGCCACCTGCTGCTGAGCCTGCTGAAAGAGCT |
| | | TTGAGAGGTAGAGACCCTGGTGCGCTCAGACCACATG |
| | | ATCCTGCTCATAGACCATTGTTGAGAGACCCTGGTCCT |
| | | AGAAGATCTGAATCTCCACCTGGTCCTGGTGGTGATGC |
| | | TTCTTTGTTGGCTAGATTGTTTGAACATCCATTGTATAG |
| | | AGTTGCTGTTCCACCATTGACTGAAGAAGATGTTTTGT |
| | | TTAATGTTAATTCTGATACTAGATTGTCTCCAAAAGCT |
| | | GCTGAAAATCCTGATTGGCCACATGCTGGTGCTGAAGG |
| | | TGCTGAATTTTTGTCTCCTGGTGAAGCGGCTGTTGATT |
| | | CCTATCCAAATTGGTTGAAATTCCACATCGGTATTAAC |
| | | CGATACGAATTGTATTCTAGACATAATCCTGCTATTGA |
| | | AGCTTTGTTGCATGATTTGTCTAGTCAGAGAATTACTT |
| | | CTGTTGCTATGAAGTCTGGTGGTACTCAGTTGAAGTTG |
| | | ATAATGACTTTTCAAAACTATGGGCAAGCTTTGTTTAA |
| | | ACCAATGAAACAAACGAGAGAACAAGAAACTCCACCTG |
| | | ATTTTTTTTATTTTTCGGATTATGAAAGACATAATGCTG |
| | | AAATTGCTGCTTTCCACTTGGACAGAATATTGGATTTT |
| | | CGCAGAGTTCCACCTGTTGCTGGTCGGATGGTTAACAT |
| | | GACTAAGGAAATTAGAGATGTTACTAGAGATAAAAAAT |
| | | TGTGGAGAACTTTTTTCATTTCTCCGGCTAATAATATTT |
| | | GTTTTTACGGGGAATGTTCTTATTATTGTTCTACTGAAC |
| | | ATGCTTTGTGTGGTAAACCTGATCAAATTGAAGGTTCT |
| | | TTGGCTGCTTTTTTGCCTGATTTGTCTTTGGCTAAAAG |
| | | AAAAACTTGGAGAAATCCATGGAGAAGATCTTATCATA |
| | | AAAGAAAAAAAGCTGAATGGGAAGTTGATCCTGATTAT |
| | | TGTGAAGAAGTTAAACAAACTCCACCATATGATTCGTC |
| | | TCATAGAATATTGGACGTCATGGATATGACGATCTTTG |
| | | ACTTTCTGATGGGGAACATGGACAGACATCACTATGAA |
| | | ACATTCGAAAAATTCGGTAATGAAACTTTTATCATCCA |
| | | TTTGGATAATGGTAGAGGTTTTGGTAAATATTCTCATG |
| | | ATGAATTGTCTATTTTGGTTCCATTGCAACAGTGTTGT |
| | | AGAATAAGGAAAAGCACTTACTTAAGATTACAACTCTT |
| | | GGCTAAAGAAGAATATAAATTGTCTTTGTTGATGGCTG |
| | | AATCTTTGAGAGGTGATCAAGTTGCTCCTGTTTTGTAT |
| | | CAACCACATTTGGAAGCTTTGGATAGAAGATTGAGAGT |
| | | TGTTTTGAAAGCTGTTAGAGATTGTGTTGAAAGAAATG |
| | | GTTTGCATTCTGTTGTTGATGATGATTTGGATACTGAA |
| | | CATAGAGCTGCTTCTGCTAGAGATTATAAAGATGATGA |
| | | TGATAAATGA |
| 371 | PpKRE2(M1- | ATGGTTCATATAGGTTTTAGAAGCTTGAAAGCCGTTTT |
| | G31)_Fam20c(R | CATTTTAGCATTGTCCTCATTGATTTTGTATGGTATTGT |
| | 32-R584)_FLAG | TACTACTTTTGATGGTAGATTGGAAAGAAGAGGTGCTA |
| | | GACCATCTGGTGAACCTGGTTGTTCTTGTGCTCAACCT |
| | | GCTGCTGAAGTCGCTGCTCCTGGTTGGGCTCAAGTTAG |
| | | AGGTAGACCTGGTGAACCACCTGCTGCTTCGTCAGCTG |
| | | CTGGTGATGCTGGTTGGCCAAATAAACATACTTTGAGA |
| | | ATTTTGCAAGATTTTTCTTCTGATCCATCTTCTAATTTA |
| | | TCTAGTCACTCTTTGGAAAAATTGCCACCTGCTGCTGA |
| | | GCCTGCTGAAAGAGCTTTGAGAGGTAGAGACCCTGGT |
| | | GCGCTCAGACCACATGATCCTGCTCATAGACCATTGTT |
| | | GAGAGACCCTGGTCCTAGAAGATCTGAATCTCCACCTG |
| | | GTCCTGGTGGTGATGCTTCTTTGTTGGCTAGATTGTTT |
| | | GAACATCCATTGTATAGAGTTGCTGTTCCACCATTGAC |
| | | TGAAGAAGATGTTTTGTTTAATGTTAATTCTGATACTA |
| | | GATTGTCTCCAAAAGCTGCTGAAAATCCTGATTGGCCA |
| | | CATGCTGGTGCTGAAGGTGCTGAATTTTTGTCTCCTGG |
| | | TGAAGCGGCTGTTGATTCCTATCCAAATTGGTTGAAAT |
| | | TCCACATCGGTATTAACCGATACGAATTGTATTCTAGA |
| | | CATAATCCTGCTATTGAAGCTTTGTTGCATGATTTGTCT |
| | | AGTCAGAGAATTACTTCTGTTGCTATGAAGTCTGGTGG |
| | | TACTCAGTTGAAGTTGATAATGACTTTTCAAAACTATG |
| | | GGCAAGCTTTGTTTAAACCAATGAAACAAACGAGAGAA |
| | | CAAGAAACTCCACCTGATTTTTTTTATTTTTCGGATTAT |
| | | GAAAGACATAATGCTGAAATTGCTGCTTTCCACTTGGA |
| | | CAGAATATTGGATTTTCGCAGAGTTCCACCTGTTGCTG |
| | | GTCGGATGGTTAACATGACTAAGGAAATTAGAGATGTT |
| | | ACTAGAGATAAAAAATTGTGGAGAACTTTTTTCATTTC |
| | | TCCGGCTAATAATATTTGTTTTTACGGGGAATGTTCTT |
| | | ATTATTGTTCTACTGAACATGCTTTGTGTGGTAAACCT |
| | | GATCAAATTGAAGGTTCTTTGGCTGCTTTTTTGCCTGA |
| | | TTTGTCTTTGGCTAAAAGAAAAACTTGGAGAAATCCAT |
| | | GGAGAAGATCTTATCATAAAAGAAAAAAAGCTGAATGG |
| | | GAAGTTGATCCTGATTATTGTGAAGAAGTTAAACAAAC |
| | | TCCACCATATGATTCGTCTCATAGAATATTGGACGTCA |
| | | TGGATATGACGATCTTTGACTTTCTGATGGGGAACATG |
| | | GACAGACATCACTATGAAACATTCGAAAAATTCGGTAA |
| | | TGAAACTTTTATCATCCATTTGGATAATGGTAGAGGTT |
| | | TTGGTAAATATTCTCATGATGAATTGTCTATTTTGGTTC |
| | | CATTGCAACAGTGTTGTAGAATAAGGAAAAGCACTTAC |
| | | TTAAGATTACAACTCTTGGCTAAAGAAGAATATAAATT |
| | | GTCTTTGTTGATGGCTGAATCTTTGAGAGGTGATCAAG |
| | | TTGCTCCTGTTTTGTATCAACCACATTTGGAAGCTTTG |
| | | GATAGAAGATTGAGAGTTGTTTTGAAAGCTGTTAGAGA |
| | | TTGTGTTGAAAGAAATGGTTTGCATTCTGTTGTTGATG |
| | | ATGATTTGGATACTGAACATAGAGCTGCTTCTGCTAGA |
| | | GATTATAAAGATGATGATGATAAATGA |
| 372 | PpKRE2(M1- | ATGGTTCATATAGGTTTTAGAAGCTTGAAAGCCGTTTT |
| | D84)_Fam20c(R | CATTTTAGCATTGTCCTCATTGATTTTGTATGGTATTGT |
| | 32-R584)_FLAG | TACTACTTTTGATGGTTCTAGAGCTTCTAGATATCAAC |
| | | CACCATATGTTAATCATTCTCAAGATCCATTGTACCATT |
| | | CTGGTAACTCCTATAATAGAGAAAACGCGACTTTTGTT |
| | | ACCTTGTGTAGAAATGAAGATTTGTATTCTATTATCCA |
| | | ATCTATCAAGAAAGTCGAAGACAGATTGGAAAGAAGA |
| | | GGTGCTAGACCATCTGGTGAACCTGGTTGTTCTTGTGC |
| | | TCAACCTGCTGCTGAAGTCGCTGCTCCTGGTTGGGCTC |
| | | AAGTTAGAGGTAGACCTGGTGAACCACCTGCTGCTTCG |
| | | TCAGCTGCTGGTGATGCTGGTTGGCCAAATAAACATAC |
| | | TTTGAGAATTTTGCAAGATTTTTCTTCTGATCCATCTTC |
| | | TAATTTATCTAGTCACTCTTTGGAAAAATTGCCACCTG |
| | | CTGCTGAGCCTGCTGAAAGAGCTTTGAGAGGTAGAGA |
| | | CCCTGGTGCGCTCAGACCACATGATCCTGCTCATAGAC |
| | | CATTGTTGAGAGACCCTGGTCCTAGAAGATCTGAATCT |
| | | CCACCTGGTCCTGGTGGTGATGCTTCTTTGTTGGCTAG |
| | | ATTGTTTGAACATCCATTGTATAGAGTTGCTGTTCCAC |
| | | CATTGACTGAAGAAGATGTTTTGTTTAATGTTAATTCT |
| | | GATACTAGATTGTCTCCAAAAGCTGCTGAAAATCCTGA |
| | | TTGGCCACATGCTGGTGCTGAAGGTGCTGAATTTTTGT |
| | | CTCCTGGTGAAGCGGCTGTTGATTCCTATCCAAATTGG |
| | | TTGAAATTCCACATCGGTATTAACCGATACGAATTGTA |
| | | TTCTAGACATAATCCTGCTATTGAAGCTTTGTTGCATG |
| | | ATTTGTCTAGTCAGAGAATTACTTCTGTTGCTATGAAG |
| | | TCTGGTGGTACTCAGTTGAAGTTGATAATGACTTTTCA |
| | | AAACTATGGGCAAGCTTTGTTTAAACCAATGAAACAAA |
| | | CGAGAGAACAAGAAACTCCACCTGATTTTTTTTATTTTT |
| | | CGGATTATGAAAGACATAATGCTGAAATTGCTGCTTTC |
| | | CACTTGGACAGAATATTGGATTTTCGCAGAGTTCCACC |
| | | TGTTGCTGGTCGGATGGTTAACATGACTAAGGAAATTA |
| | | GAGATGTTACTAGAGATAAAAAATTGTGGAGAACTTTT |
| | | TTCATTTCTCCGGCTAATAATATTTGTTTTTACGGGGAA |
| | | TGTTCTTATTATTGTTCTACTGAACATGCTTTGTGTGGT |
| | | AAACCTGATCAAATTGAAGGTTCTTTGGCTGCTTTTTT |
| | | GCCTGATTTGTCTTTGGCTAAAAGAAAAACTTGGAGAA |
| | | ATCCATGGAGAAGATCTTATCATAAAAGAAAAAAAGCT |
| | | GAATGGGAAGTTGATCCTGATTATTGTGAAGAAGTTAA |
| | | ACAAACTCCACCATATGATTCGTCTCATAGAATATTGG |
| | | ACGTCATGGATATGACGATCTTTGACTTTCTGATGGGG |
| | | AACATGGACAGACATCACTATGAAACATTCGAAAAATT |
| | | CGGTAATGAAACTTTTATCATCCATTTGGATAATGGTA |
| | | GAGGTTTTGGTAAATATTCTCATGATGAATTGTCTATTT |
| | | TGGTTCCATTGCAACAGTGTTGTAGAATAAGGAAAAGC |
| | | ACTTACTTAAGATTACAACTCTTGGCTAAAGAAGAATA |
| | | TAAATTGTCTTTGTTGATGGCTGAATCTTTGAGAGGTG |
| | | ATCAAGTTGCTCCTGTTTTGTATCAACCACATTTGGAA |
| | | GCTTTGGATAGAAGATTGAGAGTTGTTTTGAAAGCTGT |
| | | TAGAGATTGTGTTGAAAGAAATGGTTTGCATTCTGTTG |
| | | TTGATGATGATTTGGATACTGAACATAGAGCTGCTTCT |
| | | GCTAGAGATTATAAAGATGATGATGATAAATGA |
| 373 | PpKRE2(M1- | ATGGTTCATATAGGTTTTAGAAGCTTGAAAGCCGTTTT |
| | H150)_ | CATTTTAGCATTGTCCTCATTGATTTTGTATGGTATTGT |
| | Fam20c(R32- | TACTACTTTTGATGGTTCTAGAGCTTCTAGATATCAAC |
| | R584)_FLAG | CACCATATGTTAATCATTCTCAAGATCCATTGTACCATT |
| | | CTGGTAACTCCTATAATAGAGAAAACGCGACTTTTGTT |
| | | ACCTTGTGTAGAAATGAAGATTTGTATTCTATTATCCA |
| | | ATCTATCAAGAAAGTCGAAGACCGATTTAACAACAAAT |
| | | TTGCATACGATTGGGTTTTTCTGAATGAAGTTCCCTTT |
| | | ACTGATGAATTTAAAGAGAGGACTTCTGTTTTGATTTC |
| | | TGGTCAAGCTAAATATGGTTTGATTCCAAAAGAACATT |
| | | GGTCTTATCCTGATTATATTGATCAAGAAAGAGCTGCT |
| | | GAATCTAGAAGACAATTGGAAGATCAACATAGATTGGA |
| | | AAGAAGAGGTGCTAGACCATCTGGTGAACCTGGTTGTT |
| | | CTTGTGCTCAACCTGCTGCTGAAGTCGCTGCTCCTGGT |
| | | TGGGCTCAAGTTAGAGGTAGACCTGGTGAACCACCTG |
| | | CTGCTTCGTCAGCTGCTGGTGATGCTGGTTGGCCAAAT |
| | | AAACATACTTTGAGAATTTTGCAAGATTTTTCTTCTGAT |
| | | CCATCTTCTAATTTATCTAGTCACTCTTTGGAAAAATTG |
| | | CCACCTGCTGCTGAGCCTGCTGAAAGAGCTTTGAGAG |
| | | GTAGAGACCCTGGTGCGCTCAGACCACATGATCCTGCT |
| | | CATAGACCATTGTTGAGAGACCCTGGTCCTAGAAGATC |
| | | TGAATCTCCACCTGGTCCTGGTGGTGATGCTTCTTTGT |
| | | TGGCTAGATTGTTTGAACATCCATTGTATAGAGTTGCT |
| | | GTTCCACCATTGACTGAAGAAGATGTTTTGTTTAATGT |
| | | TAATTCTGATACTAGATTGTCTCCAAAAGCTGCTGAAA |
| | | ATCCTGATTGGCCACATGCTGGTGCTGAAGGTGCTGAA |
| | | TTTTTGTCTCCTGGTGAAGCGGCTGTTGATTCCTATCC |
| | | AAATTGGTTGAAATTCCACATCGGTATTAACCGATACG |
| | | AATTGTATTCTAGACATAATCCTGCTATTGAAGCTTTGT |
| | | TGCATGATTTGTCTAGTCAGAGAATTACTTCTGTTGCT |
| | | ATGAAGTCTGGTGGTACTCAGTTGAAGTTGATAATGAC |
| | | TTTTCAAAACTATGGGCAAGCTTTGTTTAAACCAATGA |
| | | AACAAACGAGAGAACAAGAAACTCCACCTGATTTTTTT |
| | | TATTTTTCGGATTATGAAAGACATAATGCTGAAATTGC |
| | | TGCTTTCCACTTGGACAGAATATTGGATTTTCGCAGAG |
| | | TTCCACCTGTTGCTGGTCGGATGGTTAACATGACTAAG |
| | | GAAATTAGAGATGTTACTAGAGATAAAAAATTGTGGAG |
| | | AACTTTTTTCATTTCTCCGGCTAATAATATTTGTTTTTA |
| | | CGGGGAATGTTCTTATTATTGTTCTACTGAACATGCTT |
| | | TGTGTGGTAAACCTGATCAAATTGAAGGTTCTTTGGCT |
| | | GCTTTTTTGCCTGATTTGTCTTTGGCTAAAAGAAAAAC |
| | | TTGGAGAAATCCATGGAGAAGATCTTATCATAAAAGAA |
| | | AAAAAGCTGAATGGGAAGTTGATCCTGATTATTGTGAA |
| | | GAAGTTAAACAAACTCCACCATATGATTCGTCTCATAG |
| | | AATATTGGACGTCATGGATATGACGATCTTTGACTTTC |
| | | TGATGGGGAACATGGACAGACATCACTATGAAACATTC |
| | | GAAAAATTCGGTAATGAAACTTTTATCATCCATTTGGA |
| | | TAATGGTAGAGGTTTTGGTAAATATTCTCATGATGAAT |
| | | TGTCTATTTTGGTTCCATTGCAACAGTGTTGTAGAATA |
| | | AGGAAAAGCACTTACTTAAGATTACAACTCTTGGCTAA |
| | | AGAAGAATATAAATTGTCTTTGTTGATGGCTGAATCTT |
| | | TGAGAGGTGATCAAGTTGCTCCTGTTTTGTATCAACCA |
| | | CATTTGGAAGCTTTGGATAGAAGATTGAGAGTTGTTTT |
| | | GAAAGCTGTTAGAGATTGTGTTGAAAGAAATGGTTTGC |
| | | ATTCTGTTGTTGATGATGATTTGGATACTGAACATAGA |
| | | GCTGCTTCTGCTAGAGATTATAAAGATGATGATGATAA |
| | | ATGA |
| 374 | ScMNN2(M1- | ATGTTGCTAACTAAACGTTTCTCTAAGTTGTTCAAGTT |
| | S36)_Fam20c | GACGTTCATTGTTTTGATACTCTGTGGTTTGTTTGTTAT |
| | (R32-R584) | CACTAATAAATACATGGATGAAAATACTTCTAGATTGG |
| | FLAG | AAAGAAGAGGTGCTAGACCATCTGGTGAACCTGGTTGT |
| | | TCTTGTGCTCAACCTGCTGCTGAAGTCGCTGCTCCTGG |
| | | TTGGGCTCAAGTTAGAGGTAGACCTGGTGAACCACCTG |
| | | CTGCTTCGTCAGCTGCTGGTGATGCTGGTTGGCCAAAT |
| | | AAACATACTTTGAGAATTTTGCAAGATTTTTCTTCTGAT |
| | | CCATCTTCTAATTTATCTAGTCACTCTTTGGAAAAATTG |
| | | CCACCTGCTGCTGAGCCTGCTGAAAGAGCTTTGAGAG |
| | | GTAGAGACCCTGGTGCGCTCAGACCACATGATCCTGCT |
| | | CATAGACCATTGTTGAGAGACCCTGGTCCTAGAAGATC |
| | | TGAATCTCCACCTGGTCCTGGTGGTGATGCTTCTTTGT |
| | | TGGCTAGATTGTTTGAACATCCATTGTATAGAGTTGCT |
| | | GTTCCACCATTGACTGAAGAAGATGTTTTGTTTAATGT |
| | | TAATTCTGATACTAGATTGTCTCCAAAAGCTGCTGAAA |
| | | ATCCTGATTGGCCACATGCTGGTGCTGAAGGTGCTGAA |
| | | TTTTTGTCTCCTGGTGAAGCGGCTGTTGATTCCTATCC |
| | | AAATTGGTTGAAATTCCACATCGGTATTAACCGATACG |
| | | AATTGTATTCTAGACATAATCCTGCTATTGAAGCTTTGT |
| | | TGCATGATTTGTCTAGTCAGAGAATTACTTCTGTTGCT |
| | | ATGAAGTCTGGTGGTACTCAGTTGAAGTTGATAATGAC |
| | | TTTTCAAAACTATGGGCAAGCTTTGTTTAAACCAATGA |
| | | AACAAACGAGAGAACAAGAAACTCCACCTGATTTTTTT |
| | | TATTTTTCGGATTATGAAAGACATAATGCTGAAATTGC |
| | | TGCTTTCCACTTGGACAGAATATTGGATTTTCGCAGAG |
| | | TTCCACCTGTTGCTGGTCGGATGGTTAACATGACTAAG |
| | | GAAATTAGAGATGTTACTAGAGATAAAAAATTGTGGAG |
| | | AACTTTTTTCATTTCTCCGGCTAATAATATTTGTTTTTA |
| | | CGGGGAATGTTCTTATTATTGTTCTACTGAACATGCTT |
| | | TGTGTGGTAAACCTGATCAAATTGAAGGTTCTTTGGCT |
| | | GCTTTTTTGCCTGATTTGTCTTTGGCTAAAAGAAAAAC |
| | | TTGGAGAAATCCATGGAGAAGATCTTATCATAAAAGAA |
| | | AAAAAGCTGAATGGGAAGTTGATCCTGATTATTGTGAA |
| | | GAAGTTAAACAAACTCCACCATATGATTCGTCTCATAG |
| | | AATATTGGACGTCATGGATATGACGATCTTTGACTTTC |
| | | TGATGGGGAACATGGACAGACATCACTATGAAACATTC |
| | | GAAAAATTCGGTAATGAAACTTTTATCATCCATTTGGA |
| | | TAATGGTAGAGGTTTTGGTAAATATTCTCATGATGAAT |
| | | TGTCTATTTTGGTTCCATTGCAACAGTGTTGTAGAATA |
| | | AGGAAAAGCACTTACTTAAGATTACAACTCTTGGCTAA |
| | | AGAAGAATATAAATTGTCTTTGTTGATGGCTGAATCTT |
| | | TGAGAGGTGATCAAGTTGCTCCTGTTTTGTATCAACCA |
| | | CATTTGGAAGCTTTGGATAGAAGATTGAGAGTTGTTTT |
| | | GAAAGCTGTTAGAGATTGTGTTGAAAGAAATGGTTTGC |
| | | ATTCTGTTGTTGATGATGATTTGGATACTGAACATAGA |
| | | GCTGCTTCTGCTAGAGATTATAAAGATGATGATGATAA |
| | | ATGA |
| 375 | ScMNN2(M1- | ATGTTGCTAACTAAACGTTTCTCTAAGTTGTTCAAGTT |
| | P97)_Fam20c(R3 | GACGTTCATTGTTTTGATACTCTGTGGTTTGTTTGTTAT |
| | 2-R584)_FLAG | CACTAATAAATACATGGATGAAAATACTTCTGTAAAGG |
| | | AGTATAAAGAGTACCTAGACAGATATGTACAGTCTTAT |
| | | TCCAATAAATATTCTTCTTCTTCTGATGCTGCTTCTGCT |
| | | GATGATTCTACTCCCTTGCGAGACAATGATGAAGCCGG |
| | | TAACGAAAAACTCAAATCCTTTTACAATAATGTTTTTAA |
| | | CTTTTTGATGGTTGATTCACCAAGATTGGAAAGAAGAG |
| | | GTGCTAGACCATCTGGTGAACCTGGTTGTTCTTGTGCT |
| | | CAACCTGCTGCTGAAGTCGCTGCTCCTGGTTGGGCTCA |
| | | AGTTAGAGGTAGACCTGGTGAACCACCTGCTGCTTCGT |
| | | CAGCTGCTGGTGATGCTGGTTGGCCAAATAAACATACT |
| | | TTGAGAATTTTGCAAGATTTTTCTTCTGATCCATCTTCT |
| | | AATTTATCTAGTCACTCTTTGGAAAAATTGCCACCTGC |
| | | TGCTGAGCCTGCTGAAAGAGCTTTGAGAGGTAGAGAC |
| | | CCTGGTGCGCTCAGACCACATGATCCTGCTCATAGACC |
| | | ATTGTTGAGAGACCCTGGTCCTAGAAGATCTGAATCTC |
| | | CACCTGGTCCTGGTGGTGATGCTTCTTTGTTGGCTAGA |
| | | TTGTTTGAACATCCATTGTATAGAGTTGCTGTTCCACC |
| | | ATTGACTGAAGAAGATGTTTTGTTTAATGTTAATTCTG |
| | | ATACTAGATTGTCTCCAAAAGCTGCTGAAAATCCTGAT |
| | | TGGCCACATGCTGGTGCTGAAGGTGCTGAATTTTTGTC |
| | | TCCTGGTGAAGCGGCTGTTGATTCCTATCCAAATTGGT |
| | | TGAAATTCCACATCGGTATTAACCGATACGAATTGTAT |
| | | TCTAGACATAATCCTGCTATTGAAGCTTTGTTGCATGA |
| | | TTTGTCTAGTCAGAGAATTACTTCTGTTGCTATGAAGT |
| | | CTGGTGGTACTCAGTTGAAGTTGATAATGACTTTTCAA |
| | | AACTATGGGCAAGCTTTGTTTAAACCAATGAAACAAAC |
| | | GAGAGAACAAGAAACTCCACCTGATTTTTTTTATTTTTC |
| | | GGATTATGAAAGACATAATGCTGAAATTGCTGCTTTCC |
| | | ACTTGGACAGAATATTGGATTTTCGCAGAGTTCCACCT |
| | | GTTGCTGGTCGGATGGTTAACATGACTAAGGAAATTAG |
| | | AGATGTTACTAGAGATAAAAAATTGTGGAGAACTTTTT |
| | | TCATTTCTCCGGCTAATAATATTTGTTTTTACGGGGAAT |
| | | GTTCTTATTATTGTTCTACTGAACATGCTTTGTGTGGTA |
| | | AACCTGATCAAATTGAAGGTTCTTTGGCTGCTTTTTTG |
| | | CCTGATTTGTCTTTGGCTAAAAGAAAAACTTGGAGAAA |
| | | TCCATGGAGAAGATCTTATCATAAAAGAAAAAAAGCTG |
| | | AATGGGAAGTTGATCCTGATTATTGTGAAGAAGTTAAA |
| | | CAAACTCCACCATATGATTCGTCTCATAGAATATTGGA |
| | | CGTCATGGATATGACGATCTTTGACTTTCTGATGGGGA |
| | | ACATGGACAGACATCACTATGAAACATTCGAAAAATTC |
| | | GGTAATGAAACTTTTATCATCCATTTGGATAATGGTAG |
| | | AGGTTTTGGTAAATATTCTCATGATGAATTGTCTATTTT |
| | | GGTTCCATTGCAACAGTGTTGTAGAATAAGGAAAAGCA |
| | | CTTACTTAAGATTACAACTCTTGGCTAAAGAAGAATAT |
| | | AAATTGTCTTTGTTGATGGCTGAATCTTTGAGAGGTGA |
| | | TCAAGTTGCTCCTGTTTTGTATCAACCACATTTGGAAG |
| | | CTTTGGATAGAAGATTGAGAGTTGTTTTGAAAGCTGTT |
| | | AGAGATTGTGTTGAAAGAAATGGTTTGCATTCTGTTGT |
| | | TGATGATGATTTGGATACTGAACATAGAGCTGCTTCTG |
| | | CTAGAGATTATAAAGATGATGATGATAAATGA |
| 376 | ScMINN2(M1- | ATGTTGCTAACTAAACGTTTCTCTAAGTTGTTCAAGTT |
| | S150)_Fam20c(R | GACGTTCATTGTTTTGATACTCTGTGGTTTGTTTGTTAT |
| | 32-R584)_FLAG | CACTAATAAATACATGGATGAAAATACTTCTGTAAAGG |
| | | AGTATAAAGAGTACCTAGACAGATATGTACAGTCTTAT |
| | | TCCAATAAATATTCTTCTTCTTCTGATGCTGCTTCTGCT |
| | | GATGATTCTACTCCCTTGCGAGACAATGATGAAGCCGG |
| | | TAACGAAAAACTCAAATCCTTTTACAATAATGTTTTTAA |
| | | CTTTTTGATGGTTGATTCACCAAAAGGTTCTACTGCTA |
| | | AACAATATAATGAAGCTTGTTTGTTGAAAGGTGATATT |
| | | GGAGATAGACCTGATCATTATAAGGATTTGTACAAATT |
| | | GTCTGCTAAAGAATTGTCTAAATGTTTGGAATTGTCTC |
| | | CTGATGAAGTTGCTTCTTTGACTAAATCTAGATTGGAA |
| | | AGAAGAGGTGCTAGACCATCTGGTGAACCTGGTTGTTC |
| | | TTGTGCTCAACCTGCTGCTGAAGTCGCTGCTCCTGGTT |
| | | GGGCTCAAGTTAGAGGTAGACCTGGTGAACCACCTGC |
| | | TGCTTCGTCAGCTGCTGGTGATGCTGGTTGGCCAAATA |
| | | AACATACTTTGAGAATTTTGCAAGATTTTTCTTCTGATC |
| | | CATCTTCTAATTTATCTAGTCACTCTTTGGAAAAATTGC |
| | | CACCTGCTGCTGAGCCTGCTGAAAGAGCTTTGAGAGGT |
| | | AGAGACCCTGGTGCGCTCAGACCACATGATCCTGCTCA |
| | | TAGACCATTGTTGAGAGACCCTGGTCCTAGAAGATCTG |
| | | AATCTCCACCTGGTCCTGGTGGTGATGCTTCTTTGTTG |
| | | GCTAGATTGTTTGAACATCCATTGTATAGAGTTGCTGT |
| | | TCCACCATTGACTGAAGAAGATGTTTTGTTTAATGTTA |
| | | ATTCTGATACTAGATTGTCTCCAAAAGCTGCTGAAAAT |
| | | CCTGATTGGCCACATGCTGGTGCTGAAGGTGCTGAATT |
| | | TTTGTCTCCTGGTGAAGCGGCTGTTGATTCCTATCCAA |
| | | ATTGGTTGAAATTCCACATCGGTATTAACCGATACGAA |
| | | TTGTATTCTAGACATAATCCTGCTATTGAAGCTTTGTTG |
| | | CATGATTTGTCTAGTCAGAGAATTACTTCTGTTGCTAT |
| | | GAAGTCTGGTGGTACTCAGTTGAAGTTGATAATGACTT |
| | | TTCAAAACTATGGGCAAGCTTTGTTTAAACCAATGAAA |
| | | CAAACGAGAGAACAAGAAACTCCACCTGATTTTTTTTA |
| | | TTTTTCGGATTATGAAAGACATAATGCTGAAATTGCTG |
| | | CTTTCCACTTGGACAGAATATTGGATTTTCGCAGAGTT |
| | | CCACCTGTTGCTGGTCGGATGGTTAACATGACTAAGGA |
| | | AATTAGAGATGTTACTAGAGATAAAAAATTGTGGAGAA |
| | | CTTTTTTCATTTCTCCGGCTAATAATATTTGTTTTTACG |
| | | GGGAATGTTCTTATTATTGTTCTACTGAACATGCTTTGT |
| | | GTGGTAAACCTGATCAAATTGAAGGTTCTTTGGCTGCT |
| | | TTTTTGCCTGATTTGTCTTTGGCTAAAAGAAAAACTTG |
| | | GAGAAATCCATGGAGAAGATCTTATCATAAAAGAAAAA |
| | | AAGCTGAATGGGAAGTTGATCCTGATTATTGTGAAGAA |
| | | GTTAAACAAACTCCACCATATGATTCGTCTCATAGAAT |
| | | ATTGGACGTCATGGATATGACGATCTTTGACTTTCTGA |
| | | TGGGGAACATGGACAGACATCACTATGAAACATTCGAA |
| | | AAATTCGGTAATGAAACTTTTATCATCCATTTGGATAAT |
| | | GGTAGAGGTTTTGGTAAATATTCTCATGATGAATTGTC |
| | | TATTTTGGTTCCATTGCAACAGTGTTGTAGAATAAGGA |
| | | AAAGCACTTACTTAAGATTACAACTCTTGGCTAAAGAA |
| | | GAATATAAATTGTCTTTGTTGATGGCTGAATCTTTGAG |
| | | AGGTGATCAAGTTGCTCCTGTTTTGTATCAACCACATT |
| | | TGGAAGCTTTGGATAGAAGATTGAGAGTTGTTTTGAAA |
| | | GCTGTTAGAGATTGTGTTGAAAGAAATGGTTTGCATTC |
| | | TGTTGTTGATGATGATTTGGATACTGAACATAGAGCTG |
| | | CTTCTGCTAGAGATTATAAAGATGATGATGATAAATGA |
| 377 | ScMNN1(M1- | ATGTTGGCTTTGAGAAGATTTATCTTGAATCAGAGATC |
| | A42)_Fam20c(R | TTTGAGATCTTGTACTATACCAATATTGGTTGGGGCTT |
| | 32-R584)_FLAG | TGATTATTATTTTGGTTTTGTTTCAATTGGTCACTCATA |
| | | GAAACGATGCTAGATTGGAAAGAAGAGGTGCTAGACC |
| | | ATCTGGTGAACCTGGTTGTTCTTGTGCTCAACCTGCTG |
| | | CTGAAGTCGCTGCTCCTGGTTGGGCTCAAGTTAGAGGT |
| | | AGACCTGGTGAACCACCTGCTGCTTCGTCAGCTGCTGG |
| | | TGATGCTGGTTGGCCAAATAAACATACTTTGAGAATTT |
| | | TGCAAGATTTTTCTTCTGATCCATCTTCTAATTTATCTA |
| | | GTCACTCTTTGGAAAAATTGCCACCTGCTGCTGAGCCT |
| | | GCTGAAAGAGCTTTGAGAGGTAGAGACCCTGGTGCGC |
| | | TCAGACCACATGATCCTGCTCATAGACCATTGTTGAGA |
| | | GACCCTGGTCCTAGAAGATCTGAATCTCCACCTGGTCC |
| | | TGGTGGTGATGCTTCTTTGTTGGCTAGATTGTTTGAAC |
| | | ATCCATTGTATAGAGTTGCTGTTCCACCATTGACTGAA |
| | | GAAGATGTTTTGTTTAATGTTAATTCTGATACTAGATTG |
| | | TCTCCAAAAGCTGCTGAAAATCCTGATTGGCCACATGC |
| | | TGGTGCTGAAGGTGCTGAATTTTTGTCTCCTGGTGAAG |
| | | CGGCTGTTGATTCCTATCCAAATTGGTTGAAATTCCAC |
| | | ATCGGTATTAACCGATACGAATTGTATTCTAGACATAA |
| | | TCCTGCTATTGAAGCTTTGTTGCATGATTTGTCTAGTC |
| | | AGAGAATTACTTCTGTTGCTATGAAGTCTGGTGGTACT |
| | | CAGTTGAAGTTGATAATGACTTTTCAAAACTATGGGCA |
| | | AGCTTTGTTTAAACCAATGAAACAAACGAGAGAACAAG |
| | | AAACTCCACCTGATTTTTTTTATTTTTCGGATTATGAAA |
| | | GACATAATGCTGAAATTGCTGCTTTCCACTTGGACAGA |
| | | ATATTGGATTTTCGCAGAGTTCCACCTGTTGCTGGTCG |
| | | GATGGTTAACATGACTAAGGAAATTAGAGATGTTACTA |
| | | GAGATAAAAAATTGTGGAGAACTTTTTTCATTTCTCCG |
| | | GCTAATAATATTTGTTTTTACGGGGAATGTTCTTATTAT |
| | | TGTTCTACTGAACATGCTTTGTGTGGTAAACCTGATCA |
| | | AATTGAAGGTTCTTTGGCTGCTTTTTTGCCTGATTTGTC |
| | | TTTGGCTAAAAGAAAAACTTGGAGAAATCCATGGAGAA |
| | | GATCTTATCATAAAAGAAAAAAAGCTGAATGGGAAGTT |
| | | GATCCTGATTATTGTGAAGAAGTTAAACAAACTCCACC |
| | | ATATGATTCGTCTCATAGAATATTGGACGTCATGGATA |
| | | TGACGATCTTTGACTTTCTGATGGGGAACATGGACAGA |
| | | CATCACTATGAAACATTCGAAAAATTCGGTAATGAAAC |
| | | TTTTATCATCCATTTGGATAATGGTAGAGGTTTTGGTA |
| | | AATATTCTCATGATGAATTGTCTATTTTGGTTCCATTGC |
| | | AACAGTGTTGTAGAATAAGGAAAAGCACTTACTTAAGA |
| | | TTACAACTCTTGGCTAAAGAAGAATATAAATTGTCTTT |
| | | GTTGATGGCTGAATCTTTGAGAGGTGATCAAGTTGCTC |
| | | CTGTTTTGTATCAACCACATTTGGAAGCTTTGGATAGA |
| | | AGATTGAGAGTTGTTTTGAAAGCTGTTAGAGATTGTGT |
| | | TGAAAGAAATGGTTTGCATTCTGTTGTTGATGATGATT |
| | | TGGATACTGAACATAGAGCTGCTTCTGCTAGAGATTAT |
| | | AAAGATGATGATGATAAATGA |
| 378 | ScMNN1(M1- | ATGTTGGCTTTGAGAAGATTTATCTTGAATCAGAGATC |
| | Q93)_Fam20c(R | TTTGAGATCTTGTACTATACCAATATTGGTTGGGGCTT |
| | 32-R584)_FLAG | TGATTATTATTTTGGTTTTGTTTCAATTGGTCACTCATA |
| | | GAAACGATGCTCTCATTAGATCCTCTAATGTTAATTCT |
| | | ACCAACAAAAAAACACTCAAGGATGCTGATCCAAAAGT |
| | | TTTGATTGAAGCTTTTGGTTCTCCTGAAGTTGATCCTG |
| | | TTGATACTATTCCTGTTTCTCCACTTGAATTGGTGCCAT |
| | | TTTACGATCAAAGATTGGAAAGAAGAGGTGCTAGACCA |
| | | TCTGGTGAACCTGGTTGTTCTTGTGCTCAACCTGCTGC |
| | | TGAAGTCGCTGCTCCTGGTTGGGCTCAAGTTAGAGGTA |
| | | GACCTGGTGAACCACCTGCTGCTTCGTCAGCTGCTGGT |
| | | GATGCTGGTTGGCCAAATAAACATACTTTGAGAATTTT |
| | | GCAAGATTTTTCTTCTGATCCATCTTCTAATTTATCTAG |
| | | TCACTCTTTGGAAAAATTGCCACCTGCTGCTGAGCCTG |
| | | CTGAAAGAGCTTTGAGAGGTAGAGACCCTGGTGCGCT |
| | | CAGACCACATGATCCTGCTCATAGACCATTGTTGAGAG |
| | | ACCCTGGTCCTAGAAGATCTGAATCTCCACCTGGTCCT |
| | | GGTGGTGATGCTTCTTTGTTGGCTAGATTGTTTGAACA |
| | | TCCATTGTATAGAGTTGCTGTTCCACCATTGACTGAAG |
| | | AAGATGTTTTGTTTAATGTTAATTCTGATACTAGATTGT |
| | | CTCCAAAAGCTGCTGAAAATCCTGATTGGCCACATGCT |
| | | GGTGCTGAAGGTGCTGAATTTTTGTCTCCTGGTGAAGC |
| | | GGCTGTTGATTCCTATCCAAATTGGTTGAAATTCCACA |
| | | TCGGTATTAACCGATACGAATTGTATTCTAGACATAAT |
| | | CCTGCTATTGAAGCTTTGTTGCATGATTTGTCTAGTCA |
| | | GAGAATTACTTCTGTTGCTATGAAGTCTGGTGGTACTC |
| | | AGTTGAAGTTGATAATGACTTTTCAAAACTATGGGCAA |
| | | GCTTTGTTTAAACCAATGAAACAAACGAGAGAACAAGA |
| | | AACTCCACCTGATTTTTTTTATTTTTCGGATTATGAAAG |
| | | ACATAATGCTGAAATTGCTGCTTTCCACTTGGACAGAA |
| | | TATTGGATTTTCGCAGAGTTCCACCTGTTGCTGGTCGG |
| | | ATGGTTAACATGACTAAGGAAATTAGAGATGTTACTAG |
| | | AGATAAAAAATTGTGGAGAACTTTTTTCATTTCTCCGG |
| | | CTAATAATATTTGTTTTTACGGGGAATGTTCTTATTATT |
| | | GTTCTACTGAACATGCTTTGTGTGGTAAACCTGATCAA |
| | | ATTGAAGGTTCTTTGGCTGCTTTTTTGCCTGATTTGTCT |
| | | TTGGCTAAAAGAAAAACTTGGAGAAATCCATGGAGAAG |
| | | ATCTTATCATAAAAGAAAAAAAGCTGAATGGGAAGTTG |
| | | ATCCTGATTATTGTGAAGAAGTTAAACAAACTCCACCA |
| | | TATGATTCGTCTCATAGAATATTGGACGTCATGGATAT |
| | | GACGATCTTTGACTTTCTGATGGGGAACATGGACAGAC |
| | | ATCACTATGAAACATTCGAAAAATTCGGTAATGAAACT |
| | | TTTATCATCCATTTGGATAATGGTAGAGGTTTTGGTAA |
| | | ATATTCTCATGATGAATTGTCTATTTTGGTTCCATTGCA |
| | | ACAGTGTTGTAGAATAAGGAAAAGCACTTACTTAAGAT |
| | | TACAACTCTTGGCTAAAGAAGAATATAAATTGTCTTTG |
| | | TTGATGGCTGAATCTTTGAGAGGTGATCAAGTTGCTCC |
| | | TGTTTTGTATCAACCACATTTGGAAGCTTTGGATAGAA |
| | | GATTGAGAGTTGTTTTGAAAGCTGTTAGAGATTGTGTT |
| | | GAAAGAAATGGTTTGCATTCTGTTGTTGATGATGATTT |
| | | GGATACTGAACATAGAGCTGCTTCTGCTAGAGATTATA |
| | | AAGATGATGATGATAAATGA |
| 379 | ScMNN1(M1- | ATGTTGGCTTTGAGAAGATTTATCTTGAATCAGAGATC |
| | G153)_ | TTTGAGATCTTGTACTATACCAATATTGGTTGGGGCTT |
| | Fam20c(R32- | TGATTATTATTTTGGTTTTGTTTCAATTGGTCACTCATA |
| | R584)_FLAG | GAAACGATGCTCTCATTAGATCCTCTAATGTTAATTCT |
| | | ACCAACAAAAAAACACTCAAGGATGCTGATCCAAAAGT |
| | | TTTGATTGAAGCTTTTGGTTCTCCTGAAGTTGATCCTG |
| | | TTGATACTATTCCTGTTTCTCCACTTGAATTGGTGCCAT |
| | | TTTACGATCAATCTATTGATACTAAGAGGTCCTCTTCAT |
| | | GGTTGATAAACAAAAAGGGTTATTATAAACACTTCAAC |
| | | GAACTGTCTTTGACGGACAGATGCAAGTTCTATTTTAG |
| | | AACATTGTATACTCTAGACGATGAGTGGACTAACTCTG |
| | | TTAAAAAATTGGAATATTCAATTAATGATAATGAAGGT |
| | | AGATTGGAAAGAAGAGGTGCTAGACCATCTGGTGAAC |
| | | CTGGTTGTTCTTGTGCTCAACCTGCTGCTGAAGTCGCT |
| | | GCTCCTGGTTGGGCTCAAGTTAGAGGTAGACCTGGTG |
| | | AACCACCTGCTGCTTCGTCAGCTGCTGGTGATGCTGGT |
| | | TGGCCAAATAAACATACTTTGAGAATTTTGCAAGATTT |
| | | TTCTTCTGATCCATCTTCTAATTTATCTAGTCACTCTTT |
| | | GGAAAAATTGCCACCTGCTGCTGAGCCTGCTGAAAGA |
| | | GCTTTGAGAGGTAGAGACCCTGGTGCGCTCAGACCAC |
| | | ATGATCCTGCTCATAGACCATTGTTGAGAGACCCTGGT |
| | | CCTAGAAGATCTGAATCTCCACCTGGTCCTGGTGGTGA |
| | | TGCTTCTTTGTTGGCTAGATTGTTTGAACATCCATTGTA |
| | | TAGAGTTGCTGTTCCACCATTGACTGAAGAAGATGTTT |
| | | TGTTTAATGTTAATTCTGATACTAGATTGTCTCCAAAAG |
| | | CTGCTGAAAATCCTGATTGGCCACATGCTGGTGCTGAA |
| | | GGTGCTGAATTTTTGTCTCCTGGTGAAGCGGCTGTTGA |
| | | TTCCTATCCAAATTGGTTGAAATTCCACATCGGTATTA |
| | | ACCGATACGAATTGTATTCTAGACATAATCCTGCTATT |
| | | GAAGCTTTGTTGCATGATTTGTCTAGTCAGAGAATTAC |
| | | TTCTGTTGCTATGAAGTCTGGTGGTACTCAGTTGAAGT |
| | | TGATAATGACTTTTCAAAACTATGGGCAAGCTTTGTTT |
| | | AAACCAATGAAACAAACGAGAGAACAAGAAACTCCACC |
| | | TGATTTTTTTTATTTTTCGGATTATGAAAGACATAATGC |
| | | TGAAATTGCTGCTTTCCACTTGGACAGAATATTGGATT |
| | | TTCGCAGAGTTCCACCTGTTGCTGGTCGGATGGTTAAC |
| | | ATGACTAAGGAAATTAGAGATGTTACTAGAGATAAAAA |
| | | ATTGTGGAGAACTTTTTTCATTTCTCCGGCTAATAATAT |
| | | TTGTTTTTACGGGGAATGTTCTTATTATTGTTCTACTGA |
| | | ACATGCTTTGTGTGGTAAACCTGATCAAATTGAAGGTT |
| | | CTTTGGCTGCTTTTTTGCCTGATTTGTCTTTGGCTAAAA |
| | | GAAAAACTTGGAGAAATCCATGGAGAAGATCTTATCAT |
| | | AAAAGAAAAAAAGCTGAATGGGAAGTTGATCCTGATTA |
| | | TTGTGAAGAAGTTAAACAAACTCCACCATATGATTCGT |
| | | CTCATAGAATATTGGACGTCATGGATATGACGATCTTT |
| | | GACTTTCTGATGGGGAACATGGACAGACATCACTATGA |
| | | AACATTCGAAAAATTCGGTAATGAAACTTTTATCATCC |
| | | ATTTGGATAATGGTAGAGGTTTTGGTAAATATTCTCAT |
| | | GATGAATTGTCTATTTTGGTTCCATTGCAACAGTGTTG |
| | | TAGAATAAGGAAAAGCACTTACTTAAGATTACAACTCT |
| | | TGGCTAAAGAAGAATATAAATTGTCTTTGTTGATGGCT |
| | | GAATCTTTGAGAGGTGATCAAGTTGCTCCTGTTTTGTA |
| | | TCAACCACATTTGGAAGCTTTGGATAGAAGATTGAGAG |
| | | TTGTTTTGAAAGCTGTTAGAGATTGTGTTGAAAGAAAT |
| | | GGTTTGCATTCTGTTGTTGATGATGATTTGGATACTGA |
| | | ACATAGAGCTGCTTCTGCTAGAGATTATAAAGATGATG |
| | | ATGATAAATGA |
| 380 | ScMNN6(M1- | ATGCATGTTTTGTTGTCTAAAAAGATTGCTAGATTTTTG |
| | P30)_Fam20c(R3 | TTGATATCTTTTGTTTTTGTTTTGGCTTTGATGGTTACC |
| | 2-R584)_FLAG | ATTAACCATCCGAGATTGGAAAGAAGAGGTGCTAGACC |
| | | ATCTGGTGAACCTGGTTGTTCTTGTGCTCAACCTGCTG |
| | | CTGAAGTCGCTGCTCCTGGTTGGGCTCAAGTTAGAGGT |
| | | AGACCTGGTGAACCACCTGCTGCTTCGTCAGCTGCTGG |
| | | TGATGCTGGTTGGCCAAATAAACATACTTTGAGAATTT |
| | | TGCAAGATTTTTCTTCTGATCCATCTTCTAATTTATCTA |
| | | GTCACTCTTTGGAAAAATTGCCACCTGCTGCTGAGCCT |
| | | GCTGAAAGAGCTTTGAGAGGTAGAGACCCTGGTGCGC |
| | | TCAGACCACATGATCCTGCTCATAGACCATTGTTGAGA |
| | | GACCCTGGTCCTAGAAGATCTGAATCTCCACCTGGTCC |
| | | TGGTGGTGATGCTTCTTTGTTGGCTAGATTGTTTGAAC |
| | | ATCCATTGTATAGAGTTGCTGTTCCACCATTGACTGAA |
| | | GAAGATGTTTTGTTTAATGTTAATTCTGATACTAGATTG |
| | | TCTCCAAAAGCTGCTGAAAATCCTGATTGGCCACATGC |
| | | TGGTGCTGAAGGTGCTGAATTTTTGTCTCCTGGTGAAG |
| | | CGGCTGTTGATTCCTATCCAAATTGGTTGAAATTCCAC |
| | | ATCGGTATTAACCGATACGAATTGTATTCTAGACATAA |
| | | TCCTGCTATTGAAGCTTTGTTGCATGATTTGTCTAGTC |
| | | AGAGAATTACTTCTGTTGCTATGAAGTCTGGTGGTACT |
| | | CAGTTGAAGTTGATAATGACTTTTCAAAACTATGGGCA |
| | | AGCTTTGTTTAAACCAATGAAACAAACGAGAGAACAAG |
| | | AAACTCCACCTGATTTTTTTTATTTTTCGGATTATGAAA |
| | | GACATAATGCTGAAATTGCTGCTTTCCACTTGGACAGA |
| | | ATATTGGATTTTCGCAGAGTTCCACCTGTTGCTGGTCG |
| | | GATGGTTAACATGACTAAGGAAATTAGAGATGTTACTA |
| | | GAGATAAAAAATTGTGGAGAACTTTTTTCATTTCTCCG |
| | | GCTAATAATATTTGTTTTTACGGGGAATGTTCTTATTAT |
| | | TGTTCTACTGAACATGCTTTGTGTGGTAAACCTGATCA |
| | | AATTGAAGGTTCTTTGGCTGCTTTTTTGCCTGATTTGTC |
| | | TTTGGCTAAAAGAAAAACTTGGAGAAATCCATGGAGAA |
| | | GATCTTATCATAAAAGAAAAAAAGCTGAATGGGAAGTT |
| | | GATCCTGATTATTGTGAAGAAGTTAAACAAACTCCACC |
| | | ATATGATTCGTCTCATAGAATATTGGACGTCATGGATA |
| | | TGACGATCTTTGACTTTCTGATGGGGAACATGGACAGA |
| | | CATCACTATGAAACATTCGAAAAATTCGGTAATGAAAC |
| | | TTTTATCATCCATTTGGATAATGGTAGAGGTTTTGGTA |
| | | AATATTCTCATGATGAATTGTCTATTTTGGTTCCATTGC |
| | | AACAGTGTTGTAGAATAAGGAAAAGCACTTACTTAAGA |
| | | TTACAACTCTTGGCTAAAGAAGAATATAAATTGTCTTT |
| | | GTTGATGGCTGAATCTTTGAGAGGTGATCAAGTTGCTC |
| | | CTGTTTTGTATCAACCACATTTGGAAGCTTTGGATAGA |
| | | AGATTGAGAGTTGTTTTGAAAGCTGTTAGAGATTGTGT |
| | | TGAAAGAAATGGTTTGCATTCTGTTGTTGATGATGATT |
| | | TGGATACTGAACATAGAGCTGCTTCTGCTAGAGATTAT |
| | | AAAGATGATGATGATAAATGA |
| 381 | ScMNN6(M1- | ATGCATGTTTTGTTGTCTAAAAAGATTGCTAGATTTTTG |
| | V85)_Fam20c(R | TTGATATCTTTTGTTTTTGTTTTGGCTTTGATGGTTACC |
| | 32-R584)_FLAG | ATTAACCATCCGAAGACTAAACAAATGTCTGAACAATA |
| | | TGTTACTCCATATTTGCCAAAATCTTTGCAACCAATTGC |
| | | TAAAATTTCTGCTGAAGAACAAAGAAGAATTCAATCTG |
| | | AACAAGAAGAAGCTGAATTGAAACAATCTTTGGAAGGT |
| | | GAAGCAATAAGAAATGCTACCGTTAGATTGGAAAGAAG |
| | | AGGTGCTAGACCATCTGGTGAACCTGGTTGTTCTTGTG |
| | | CTCAACCTGCTGCTGAAGTCGCTGCTCCTGGTTGGGCT |
| | | CAAGTTAGAGGTAGACCTGGTGAACCACCTGCTGCTTC |
| | | GTCAGCTGCTGGTGATGCTGGTTGGCCAAATAAACATA |
| | | CTTTGAGAATTTTGCAAGATTTTTCTTCTGATCCATCTT |
| | | CTAATTTATCTAGTCACTCTTTGGAAAAATTGCCACCT |
| | | GCTGCTGAGCCTGCTGAAAGAGCTTTGAGAGGTAGAG |
| | | ACCCTGGTGCGCTCAGACCACATGATCCTGCTCATAGA |
| | | CCATTGTTGAGAGACCCTGGTCCTAGAAGATCTGAATC |
| | | TCCACCTGGTCCTGGTGGTGATGCTTCTTTGTTGGCTA |
| | | GATTGTTTGAACATCCATTGTATAGAGTTGCTGTTCCA |
| | | CCATTGACTGAAGAAGATGTTTTGTTTAATGTTAATTCT |
| | | GATACTAGATTGTCTCCAAAAGCTGCTGAAAATCCTGA |
| | | TTGGCCACATGCTGGTGCTGAAGGTGCTGAATTTTTGT |
| | | CTCCTGGTGAAGCGGCTGTTGATTCCTATCCAAATTGG |
| | | TTGAAATTCCACATCGGTATTAACCGATACGAATTGTA |
| | | TTCTAGACATAATCCTGCTATTGAAGCTTTGTTGCATG |
| | | ATTTGTCTAGTCAGAGAATTACTTCTGTTGCTATGAAG |
| | | TCTGGTGGTACTCAGTTGAAGTTGATAATGACTTTTCA |
| | | AAACTATGGGCAAGCTTTGTTTAAACCAATGAAACAAA |
| | | CGAGAGAACAAGAAACTCCACCTGATTTTTTTTATTTTT |
| | | CGGATTATGAAAGACATAATGCTGAAATTGCTGCTTTC |
| | | CACTTGGACAGAATATTGGATTTTCGCAGAGTTCCACC |
| | | TGTTGCTGGTCGGATGGTTAACATGACTAAGGAAATTA |
| | | GAGATGTTACTAGAGATAAAAAATTGTGGAGAACTTTT |
| | | TTCATTTCTCCGGCTAATAATATTTGTTTTTACGGGGAA |
| | | TGTTCTTATTATTGTTCTACTGAACATGCTTTGTGTGGT |
| | | AAACCTGATCAAATTGAAGGTTCTTTGGCTGCTTTTTT |
| | | GCCTGATTTGTCTTTGGCTAAAAGAAAAACTTGGAGAA |
| | | ATCCATGGAGAAGATCTTATCATAAAAGAAAAAAAGCT |
| | | GAATGGGAAGTTGATCCTGATTATTGTGAAGAAGTTAA |
| | | ACAAACTCCACCATATGATTCGTCTCATAGAATATTGG |
| | | ACGTCATGGATATGACGATCTTTGACTTTCTGATGGGG |
| | | AACATGGACAGACATCACTATGAAACATTCGAAAAATT |
| | | CGGTAATGAAACTTTTATCATCCATTTGGATAATGGTA |
| | | GAGGTTTTGGTAAATATTCTCATGATGAATTGTCTATTT |
| | | TGGTTCCATTGCAACAGTGTTGTAGAATAAGGAAAAGC |
| | | ACTTACTTAAGATTACAACTCTTGGCTAAAGAAGAATA |
| | | TAAATTGTCTTTGTTGATGGCTGAATCTTTGAGAGGTG |
| | | ATCAAGTTGCTCCTGTTTTGTATCAACCACATTTGGAA |
| | | GCTTTGGATAGAAGATTGAGAGTTGTTTTGAAAGCTGT |
| | | TAGAGATTGTGTTGAAAGAAATGGTTTGCATTCTGTTG |
| | | TTGATGATGATTTGGATACTGAACATAGAGCTGCTTCT |
| | | GCTAGAGATTATAAAGATGATGATGATAAATGA |
| 382 | ScMNN6(M1- | ATGCATGTTTTGTTGTCTAAAAAGATTGCTAGATTTTTG |
| | E160)_Fam20c(R | TTGATATCTTTTGTTTTTGTTTTGGCTTTGATGGTTACC |
| | 32-R584)_FLAG | ATTAACCATCCGAAGACTAAACAAATGTCTGAACAATA |
| | | TGTTACTCCATATTTGCCAAAATCTTTGCAACCAATTGC |
| | | TAAAATTTCTGCTGAAGAACAAAGAAGAATTCAATCTG |
| | | AACAAGAAGAAGCTGAATTGAAACAATCTTTGGAAGGT |
| | | GAAGCAATAAGAAATGCTACCGTTAACGCCATTAAAGA |
| | | AAAAATTAAATCTTATGGTGGTAATGAAACTACTTTGG |
| | | GTTTTATGGTTCCATCTTATATTAATCATAGAGGTTCTC |
| | | CACCCAAAGCTTGCTTCGTTTCATTGATCACAGAAAGG |
| | | GACTCTATGACTCAAATCTTGCAATCTATAGATGAGGT |
| | | CCAAGTCAAGTTTAACAAAAATTTTGCTTATCCATGGG |
| | | TTTTTATTTCTCAAGGTGAAAGATTGGAAAGAAGAGGT |
| | | GCTAGACCATCTGGTGAACCTGGTTGTTCTTGTGCTCA |
| | | ACCTGCTGCTGAAGTCGCTGCTCCTGGTTGGGCTCAAG |
| | | TTAGAGGTAGACCTGGTGAACCACCTGCTGCTTCGTCA |
| | | GCTGCTGGTGATGCTGGTTGGCCAAATAAACATACTTT |
| | | GAGAATTTTGCAAGATTTTTCTTCTGATCCATCTTCTAA |
| | | TTTATCTAGTCACTCTTTGGAAAAATTGCCACCTGCTG |
| | | CTGAGCCTGCTGAAAGAGCTTTGAGAGGTAGAGACCC |
| | | TGGTGCGCTCAGACCACATGATCCTGCTCATAGACCAT |
| | | TGTTGAGAGACCCTGGTCCTAGAAGATCTGAATCTCCA |
| | | CCTGGTCCTGGTGGTGATGCTTCTTTGTTGGCTAGATT |
| | | GTTTGAACATCCATTGTATAGAGTTGCTGTTCCACCAT |
| | | TGACTGAAGAAGATGTTTTGTTTAATGTTAATTCTGAT |
| | | ACTAGATTGTCTCCAAAAGCTGCTGAAAATCCTGATTG |
| | | GCCACATGCTGGTGCTGAAGGTGCTGAATTTTTGTCTC |
| | | CTGGTGAAGCGGCTGTTGATTCCTATCCAAATTGGTTG |
| | | AAATTCCACATCGGTATTAACCGATACGAATTGTATTC |
| | | TAGACATAATCCTGCTATTGAAGCTTTGTTGCATGATT |
| | | TGTCTAGTCAGAGAATTACTTCTGTTGCTATGAAGTCT |
| | | GGTGGTACTCAGTTGAAGTTGATAATGACTTTTCAAAA |
| | | CTATGGGCAAGCTTTGTTTAAACCAATGAAACAAACGA |
| | | GAGAACAAGAAACTCCACCTGATTTTTTTTATTTTTCG |
| | | GATTATGAAAGACATAATGCTGAAATTGCTGCTTTCCA |
| | | CTTGGACAGAATATTGGATTTTCGCAGAGTTCCACCTG |
| | | TTGCTGGTCGGATGGTTAACATGACTAAGGAAATTAGA |
| | | GATGTTACTAGAGATAAAAAATTGTGGAGAACTTTTTT |
| | | CATTTCTCCGGCTAATAATATTTGTTTTTACGGGGAAT |
| | | GTTCTTATTATTGTTCTACTGAACATGCTTTGTGTGGTA |
| | | AACCTGATCAAATTGAAGGTTCTTTGGCTGCTTTTTTG |
| | | CCTGATTTGTCTTTGGCTAAAAGAAAAACTTGGAGAAA |
| | | TCCATGGAGAAGATCTTATCATAAAAGAAAAAAAGCTG |
| | | AATGGGAAGTTGATCCTGATTATTGTGAAGAAGTTAAA |
| | | CAAACTCCACCATATGATTCGTCTCATAGAATATTGGA |
| | | CGTCATGGATATGACGATCTTTGACTTTCTGATGGGGA |
| | | ACATGGACAGACATCACTATGAAACATTCGAAAAATTC |
| | | GGTAATGAAACTTTTATCATCCATTTGGATAATGGTAG |
| | | AGGTTTTGGTAAATATTCTCATGATGAATTGTCTATTTT |
| | | GGTTCCATTGCAACAGTGTTGTAGAATAAGGAAAAGCA |
| | | CTTACTTAAGATTACAACTCTTGGCTAAAGAAGAATAT |
| | | AAATTGTCTTTGTTGATGGCTGAATCTTTGAGAGGTGA |
| | | TCAAGTTGCTCCTGTTTTGTATCAACCACATTTGGAAG |
| | | CTTTGGATAGAAGATTGAGAGTTGTTTTGAAAGCTGTT |
| | | AGAGATTGTGTTGAAAGAAATGGTTTGCATTCTGTTGT |
| | | TGATGATGATTTGGATACTGAACATAGAGCTGCTTCTG |
| | | CTAGAGATTATAAAGATGATGATGATAAATGA |
| 383 | Fam20c(R64- | ATGAGAGGTAGACCTGGTGAACCACCTGCTGCTTCGTC |
| | R584)_FLAG | AGCTGCTGGTGATGCTGGTTGGCCAAATAAACATACTT |
| | | TGAGAATTTTGCAAGATTTTTCTTCTGATCCATCTTCTA |
| | | ATTTATCTAGTCACTCTTTGGAAAAATTGCCACCTGCT |
| | | GCTGAGCCTGCTGAAAGAGCTTTGAGAGGTAGAGACC |
| | | CTGGTGCGCTCAGACCACATGATCCTGCTCATAGACCA |
| | | TTGTTGAGAGACCCTGGTCCTAGAAGATCTGAATCTCC |
| | | ACCTGGTCCTGGTGGTGATGCTTCTTTGTTGGCTAGAT |
| | | TGTTTGAACATCCATTGTATAGAGTTGCTGTTCCACCA |
| | | TTGACTGAAGAAGATGTTTTGTTTAATGTTAATTCTGAT |
| | | ACTAGATTGTCTCCAAAAGCTGCTGAAAATCCTGATTG |
| | | GCCACATGCTGGTGCTGAAGGTGCTGAATTTTTGTCTC |
| | | CTGGTGAAGCGGCTGTTGATTCCTATCCAAATTGGTTG |
| | | AAATTCCACATCGGTATTAACCGATACGAATTGTATTC |
| | | TAGACATAATCCTGCTATTGAAGCTTTGTTGCATGATT |
| | | TGTCTAGTCAGAGAATTACTTCTGTTGCTATGAAGTCT |
| | | GGTGGTACTCAGTTGAAGTTGATAATGACTTTTCAAAA |
| | | CTATGGGCAAGCTTTGTTTAAACCAATGAAACAAACGA |
| | | GAGAACAAGAAACTCCACCTGATTTTTTTTATTTTTCG |
| | | GATTATGAAAGACATAATGCTGAAATTGCTGCTTTCCA |
| | | CTTGGACAGAATATTGGATTTTCGCAGAGTTCCACCTG |
| | | TTGCTGGTCGGATGGTTAACATGACTAAGGAAATTAGA |
| | | GATGTTACTAGAGATAAAAAATTGTGGAGAACTTTTTT |
| | | CATTTCTCCGGCTAATAATATTTGTTTTTACGGGGAAT |
| | | GTTCTTATTATTGTTCTACTGAACATGCTTTGTGTGGTA |
| | | AACCTGATCAAATTGAAGGTTCTTTGGCTGCTTTTTTG |
| | | CCTGATTTGTCTTTGGCTAAAAGAAAAACTTGGAGAAA |
| | | TCCATGGAGAAGATCTTATCATAAAAGAAAAAAAGCTG |
| | | AATGGGAAGTTGATCCTGATTATTGTGAAGAAGTTAAA |
| | | CAAACTCCACCATATGATTCGTCTCATAGAATATTGGA |
| | | CGTCATGGATATGACGATCTTTGACTTTCTGATGGGGA |
| | | ACATGGACAGACATCACTATGAAACATTCGAAAAATTC |
| | | GGTAATGAAACTTTTATCATCCATTTGGATAATGGTAG |
| | | AGGTTTTGGTAAATATTCTCATGATGAATTGTCTATTTT |
| | | GGTTCCATTGCAACAGTGTTGTAGAATAAGGAAAAGCA |
| | | CTTACTTAAGATTACAACTCTTGGCTAAAGAAGAATAT |
| | | AAATTGTCTTTGTTGATGGCTGAATCTTTGAGAGGTGA |
| | | TCAAGTTGCTCCTGTTTTGTATCAACCACATTTGGAAG |
| | | CTTTGGATAGAAGATTGAGAGTTGTTTTGAAAGCTGTT |
| | | AGAGATTGTGTTGAAAGAAATGGTTTGCATTCTGTTGT |
| | | TGATGATGATTTGGATACTGAACATAGAGCTGCTTCTG |
| | | CTAGAGATTATAAAGATGATGATGATAAATGA |
| 384 | ScKRE(M1- | ATGGCTTTATTTTTGTCTAAAAGATTGTTGAGATTTACT |
| | 158)_Fam20c(R6 | GTTATTGCTGGTGCTGTTATTGTTCTATTGTTGACGCT |
| | 4-R584)_FLAG | CAATTCAAATTCACGAACTCAACAATATATTCCATCTTC |
| | | TATTTCTGCTGCTTTTGATTTTACTTCTGGTTCTATTTC |
| | | TCCTGAACAACAAGTTATTAGAGGTAGACCTGGTGAAC |
| | | CACCTGCTGCTTCGTCAGCTGCTGGTGATGCTGGTTGG |
| | | CCAAATAAACATACTTTGAGAATTTTGCAAGATTTTTCT |
| | | TCTGATCCATCTTCTAATTTATCTAGTCACTCTTTGGAA |
| | | AAATTGCCACCTGCTGCTGAGCCTGCTGAAAGAGCTTT |
| | | GAGAGGTAGAGACCCTGGTGCGCTCAGACCACATGAT |
| | | CCTGCTCATAGACCATTGTTGAGAGACCCTGGTCCTAG |
| | | AAGATCTGAATCTCCACCTGGTCCTGGTGGTGATGCTT |
| | | CTTTGTTGGCTAGATTGTTTGAACATCCATTGTATAGA |
| | | GTTGCTGTTCCACCATTGACTGAAGAAGATGTTTTGTT |
| | | TAATGTTAATTCTGATACTAGATTGTCTCCAAAAGCTG |
| | | CTGAAAATCCTGATTGGCCACATGCTGGTGCTGAAGGT |
| | | GCTGAATTTTTGTCTCCTGGTGAAGCGGCTGTTGATTC |
| | | CTATCCAAATTGGTTGAAATTCCACATCGGTATTAACC |
| | | GATACGAATTGTATTCTAGACATAATCCTGCTATTGAA |
| | | GCTTTGTTGCATGATTTGTCTAGTCAGAGAATTACTTC |
| | | TGTTGCTATGAAGTCTGGTGGTACTCAGTTGAAGTTGA |
| | | TAATGACTTTTCAAAACTATGGGCAAGCTTTGTTTAAA |
| | | CCAATGAAACAAACGAGAGAACAAGAAACTCCACCTGA |
| | | TTTTTTTTATTTTTCGGATTATGAAAGACATAATGCTGA |
| | | AATTGCTGCTTTCCACTTGGACAGAATATTGGATTTTC |
| | | GCAGAGTTCCACCTGTTGCTGGTCGGATGGTTAACATG |
| | | ACTAAGGAAATTAGAGATGTTACTAGAGATAAAAAATT |
| | | GTGGAGAACTTTTTTCATTTCTCCGGCTAATAATATTTG |
| | | TTTTTACGGGGAATGTTCTTATTATTGTTCTACTGAACA |
| | | TGCTTTGTGTGGTAAACCTGATCAAATTGAAGGTTCTT |
| | | TGGCTGCTTTTTTGCCTGATTTGTCTTTGGCTAAAAGA |
| | | AAAACTTGGAGAAATCCATGGAGAAGATCTTATCATAA |
| | | AAGAAAAAAAGCTGAATGGGAAGTTGATCCTGATTATT |
| | | GTGAAGAAGTTAAACAAACTCCACCATATGATTCGTCT |
| | | CATAGAATATTGGACGTCATGGATATGACGATCTTTGA |
| | | CTTTCTGATGGGGAACATGGACAGACATCACTATGAAA |
| | | CATTCGAAAAATTCGGTAATGAAACTTTTATCATCCATT |
| | | TGGATAATGGTAGAGGTTTTGGTAAATATTCTCATGAT |
| | | GAATTGTCTATTTTGGTTCCATTGCAACAGTGTTGTAG |
| | | AATAAGGAAAAGCACTTACTTAAGATTACAACTCTTGG |
| | | CTAAAGAAGAATATAAATTGTCTTTGTTGATGGCTGAA |
| | | TCTTTGAGAGGTGATCAAGTTGCTCCTGTTTTGTATCA |
| | | ACCACATTTGGAAGCTTTGGATAGAAGATTGAGAGTTG |
| | | TTTTGAAAGCTGTTAGAGATTGTGTTGAAAGAAATGGT |
| | | TTGCATTCTGTTGTTGATGATGATTTGGATACTGAACA |
| | | TAGAGCTGCTTCTGCTAGAGATTATAAAGATGATGATG |
| | | ATAAATGA |
| 385 | ScKRE2(M1- | ATGGCTTTATTTTTGTCTAAAAGATTGTTGAGATTTACT |
| | S80)_Fam20c | GTTATTGCTGGTGCTGTTATTGTTCTATTGTTGACGCT |
| | (R64-R584)_ | CAATTCAAATTCACGAACTCAACAATATATTCCATCTTC |
| | FLAG | TATTTCTGCTGCTTTTGATTTTACTTCTGGTTCTATTTC |
| | | TCCTGAACAACAAGTTATTTCTGAAGAAAATGATGCTA |
| | | AGAAATTAGAACAGTCCGCTTTGAATTCTGAAGCTTCT |
| | | GAAGATTCTAGAGGTAGACCTGGTGAACCACCTGCTGC |
| | | TTCGTCAGCTGCTGGTGATGCTGGTTGGCCAAATAAAC |
| | | ATACTTTGAGAATTTTGCAAGATTTTTCTTCTGATCCAT |
| | | CTTCTAATTTATCTAGTCACTCTTTGGAAAAATTGCCAC |
| | | CTGCTGCTGAGCCTGCTGAAAGAGCTTTGAGAGGTAG |
| | | AGACCCTGGTGCGCTCAGACCACATGATCCTGCTCATA |
| | | GACCATTGTTGAGAGACCCTGGTCCTAGAAGATCTGAA |
| | | TCTCCACCTGGTCCTGGTGGTGATGCTTCTTTGTTGGC |
| | | TAGATTGTTTGAACATCCATTGTATAGAGTTGCTGTTC |
| | | CACCATTGACTGAAGAAGATGTTTTGTTTAATGTTAAT |
| | | TCTGATACTAGATTGTCTCCAAAAGCTGCTGAAAATCC |
| | | TGATTGGCCACATGCTGGTGCTGAAGGTGCTGAATTTT |
| | | TGTCTCCTGGTGAAGCGGCTGTTGATTCCTATCCAAAT |
| | | TGGTTGAAATTCCACATCGGTATTAACCGATACGAATT |
| | | GTATTCTAGACATAATCCTGCTATTGAAGCTTTGTTGC |
| | | ATGATTTGTCTAGTCAGAGAATTACTTCTGTTGCTATG |
| | | AAGTCTGGTGGTACTCAGTTGAAGTTGATAATGACTTT |
| | | TCAAAACTATGGGCAAGCTTTGTTTAAACCAATGAAAC |
| | | AAACGAGAGAACAAGAAACTCCACCTGATTTTTTTTAT |
| | | TTTTCGGATTATGAAAGACATAATGCTGAAATTGCTGC |
| | | TTTCCACTTGGACAGAATATTGGATTTTCGCAGAGTTC |
| | | CACCTGTTGCTGGTCGGATGGTTAACATGACTAAGGAA |
| | | ATTAGAGATGTTACTAGAGATAAAAAATTGTGGAGAAC |
| | | TTTTTTCATTTCTCCGGCTAATAATATTTGTTTTTACGG |
| | | GGAATGTTCTTATTATTGTTCTACTGAACATGCTTTGTG |
| | | TGGTAAACCTGATCAAATTGAAGGTTCTTTGGCTGCTT |
| | | TTTTGCCTGATTTGTCTTTGGCTAAAAGAAAAACTTGG |
| | | AGAAATCCATGGAGAAGATCTTATCATAAAAGAAAAAA |
| | | AGCTGAATGGGAAGTTGATCCTGATTATTGTGAAGAAG |
| | | TTAAACAAACTCCACCATATGATTCGTCTCATAGAATA |
| | | TTGGACGTCATGGATATGACGATCTTTGACTTTCTGAT |
| | | GGGGAACATGGACAGACATCACTATGAAACATTCGAAA |
| | | AATTCGGTAATGAAACTTTTATCATCCATTTGGATAAT |
| 386 | ScKRE2(M1- | GGTAGAGGTTTTGGTAAATATTCTCATGATGAATTGTC |
| | D102)_ | TATTTTGGTTCCATTGCAACAGTGTTGTAGAATAAGGA |
| | Fam20c(R64- | AAAGCACTTACTTAAGATTACAACTCTTGGCTAAAGAA |
| | R584)_FLAG | GAATATAAATTGTCTTTGTTGATGGCTGAATCTTTGAG |
| | | AGGTGATCAAGTTGCTCCTGTTTTGTATCAACCACATT |
| | | TGGAAGCTTTGGATAGAAGATTGAGAGTTGTTTTGAAA |
| | | GCTGTTAGAGATTGTGTTGAAAGAAATGGTTTGCATTC |
| | | TGTTGTTGATGATGATTTGGATACTGAACATAGAGCTG |
| | | CTTCTGCTAGAGATTATAAAGATGATGATGATAAATGA |
| | | ATGGCTTTATTTTTGTCTAAAAGATTGTTGAGATTTACT |
| | | GTTATTGCTGGTGCTGTTATTGTTCTATTGTTGACGCT |
| | | CAATTCAAATTCACGAACTCAACAATATATTCCATCTTC |
| | | TATTTCTGCTGCTTTTGATTTTACTTCTGGTTCTATTTC |
| | | TCCTGAACAACAAGTTATTTCTGAAGAAAATGATGCTA |
| | | AGAAATTAGAACAGTCCGCTTTGAATTCTGAAGCTTCT |
| | | GAAGATTCTGAAGCTATGGATGAAGAATCTAAAGCTTT |
| | | GAAAGCTGCTGCTGAAAAAGCTGATGCTCCAATTGATA |
| | | GAGGTAGACCTGGTGAACCACCTGCTGCTTCGTCAGCT |
| | | GCTGGTGATGCTGGTTGGCCAAATAAACATACTTTGAG |
| | | AATTTTGCAAGATTTTTCTTCTGATCCATCTTCTAATTT |
| | | ATCTAGTCACTCTTTGGAAAAATTGCCACCTGCTGCTG |
| | | AGCCTGCTGAAAGAGCTTTGAGAGGTAGAGACCCTGG |
| | | TGCGCTCAGACCACATGATCCTGCTCATAGACCATTGT |
| | | TGAGAGACCCTGGTCCTAGAAGATCTGAATCTCCACCT |
| | | GGTCCTGGTGGTGATGCTTCTTTGTTGGCTAGATTGTT |
| | | TGAACATCCATTGTATAGAGTTGCTGTTCCACCATTGA |
| | | CTGAAGAAGATGTTTTGTTTAATGTTAATTCTGATACTA |
| | | GATTGTCTCCAAAAGCTGCTGAAAATCCTGATTGGCCA |
| | | CATGCTGGTGCTGAAGGTGCTGAATTTTTGTCTCCTGG |
| | | TGAAGCGGCTGTTGATTCCTATCCAAATTGGTTGAAAT |
| | | TCCACATCGGTATTAACCGATACGAATTGTATTCTAGA |
| | | CATAATCCTGCTATTGAAGCTTTGTTGCATGATTTGTCT |
| | | AGTCAGAGAATTACTTCTGTTGCTATGAAGTCTGGTGG |
| | | TACTCAGTTGAAGTTGATAATGACTTTTCAAAACTATG |
| | | GGCAAGCTTTGTTTAAACCAATGAAACAAACGAGAGAA |
| | | CAAGAAACTCCACCTGATTTTTTTTATTTTTCGGATTAT |
| | | GAAAGACATAATGCTGAAATTGCTGCTTTCCACTTGGA |
| | | CAGAATATTGGATTTTCGCAGAGTTCCACCTGTTGCTG |
| | | GTCGGATGGTTAACATGACTAAGGAAATTAGAGATGTT |
| | | ACTAGAGATAAAAAATTGTGGAGAACTTTTTTCATTTC |
| | | TCCGGCTAATAATATTTGTTTTTACGGGGAATGTTCTT |
| | | ATTATTGTTCTACTGAACATGCTTTGTGTGGTAAACCT |
| | | GATCAAATTGAAGGTTCTTTGGCTGCTTTTTTGCCTGA |
| | | TTTGTCTTTGGCTAAAAGAAAAACTTGGAGAAATCCAT |
| | | GGAGAAGATCTTATCATAAAAGAAAAAAAGCTGAATGG |
| | | GAAGTTGATCCTGATTATTGTGAAGAAGTTAAACAAAC |
| | | TCCACCATATGATTCGTCTCATAGAATATTGGACGTCA |
| | | TGGATATGACGATCTTTGACTTTCTGATGGGGAACATG |
| | | GACAGACATCACTATGAAACATTCGAAAAATTCGGTAA |
| | | TGAAACTTTTATCATCCATTTGGATAATGGTAGAGGTT |
| | | TTGGTAAATATTCTCATGATGAATTGTCTATTTTGGTTC |
| | | CATTGCAACAGTGTTGTAGAATAAGGAAAAGCACTTAC |
| | | TTAAGATTACAACTCTTGGCTAAAGAAGAATATAAATT |
| | | GTCTTTGTTGATGGCTGAATCTTTGAGAGGTGATCAAG |
| | | TTGCTCCTGTTTTGTATCAACCACATTTGGAAGCTTTG |
| | | GATAGAAGATTGAGAGTTGTTTTGAAAGCTGTTAGAGA |
| | | TTGTGTTGAAAGAAATGGTTTGCATTCTGTTGTTGATG |
| | | ATGATTTGGATACTGAACATAGAGCTGCTTCTGCTAGA |
| | | GATTATAAAGATGATGATGATAAATGA |
| 387 | PpKRE2(M1- | ATGGTTCATATAGGTTTTAGAAGCTTGAAAGCCGTTTT |
| | G31)_Fam20c(R | CATTTTAGCATTGTCCTCATTGATTTTGTATGGTATTGT |
| | 64-R584)_FLAG | TACTACTTTTGATGGTAGAGGTAGACCTGGTGAACCAC |
| | | CTGCTGCTTCGTCAGCTGCTGGTGATGCTGGTTGGCCA |
| | | AATAAACATACTTTGAGAATTTTGCAAGATTTTTCTTCT |
| | | GATCCATCTTCTAATTTATCTAGTCACTCTTTGGAAAAA |
| | | TTGCCACCTGCTGCTGAGCCTGCTGAAAGAGCTTTGAG |
| | | AGGTAGAGACCCTGGTGCGCTCAGACCACATGATCCT |
| | | GCTCATAGACCATTGTTGAGAGACCCTGGTCCTAGAAG |
| | | ATCTGAATCTCCACCTGGTCCTGGTGGTGATGCTTCTT |
| | | TGTTGGCTAGATTGTTTGAACATCCATTGTATAGAGTT |
| | | GCTGTTCCACCATTGACTGAAGAAGATGTTTTGTTTAA |
| | | TGTTAATTCTGATACTAGATTGTCTCCAAAAGCTGCTG |
| | | AAAATCCTGATTGGCCACATGCTGGTGCTGAAGGTGCT |
| | | GAATTTTTGTCTCCTGGTGAAGCGGCTGTTGATTCCTA |
| | | TCCAAATTGGTTGAAATTCCACATCGGTATTAACCGAT |
| | | ACGAATTGTATTCTAGACATAATCCTGCTATTGAAGCT |
| | | TTGTTGCATGATTTGTCTAGTCAGAGAATTACTTCTGTT |
| | | GCTATGAAGTCTGGTGGTACTCAGTTGAAGTTGATAAT |
| | | GACTTTTCAAAACTATGGGCAAGCTTTGTTTAAACCAA |
| | | TGAAACAAACGAGAGAACAAGAAACTCCACCTGATTTT |
| | | TTTTATTTTTCGGATTATGAAAGACATAATGCTGAAATT |
| | | GCTGCTTTCCACTTGGACAGAATATTGGATTTTCGCAG |
| | | AGTTCCACCTGTTGCTGGTCGGATGGTTAACATGACTA |
| | | AGGAAATTAGAGATGTTACTAGAGATAAAAAATTGTGG |
| | | AGAACTTTTTTCATTTCTCCGGCTAATAATATTTGTTTT |
| | | TACGGGGAATGTTCTTATTATTGTTCTACTGAACATGC |
| | | TTTGTGTGGTAAACCTGATCAAATTGAAGGTTCTTTGG |
| | | CTGCTTTTTTGCCTGATTTGTCTTTGGCTAAAAGAAAA |
| | | ACTTGGAGAAATCCATGGAGAAGATCTTATCATAAAAG |
| | | AAAAAAAGCTGAATGGGAAGTTGATCCTGATTATTGTG |
| | | AAGAAGTTAAACAAACTCCACCATATGATTCGTCTCAT |
| | | AGAATATTGGACGTCATGGATATGACGATCTTTGACTT |
| | | TCTGATGGGGAACATGGACAGACATCACTATGAAACAT |
| | | TCGAAAAATTCGGTAATGAAACTTTTATCATCCATTTG |
| | | GATAATGGTAGAGGTTTTGGTAAATATTCTCATGATGA |
| | | ATTGTCTATTTTGGTTCCATTGCAACAGTGTTGTAGAA |
| | | TAAGGAAAAGCACTTACTTAAGATTACAACTCTTGGCT |
| | | AAAGAAGAATATAAATTGTCTTTGTTGATGGCTGAATC |
| | | TTTGAGAGGTGATCAAGTTGCTCCTGTTTTGTATCAAC |
| | | CACATTTGGAAGCTTTGGATAGAAGATTGAGAGTTGTT |
| | | TTGAAAGCTGTTAGAGATTGTGTTGAAAGAAATGGTTT |
| | | GCATTCTGTTGTTGATGATGATTTGGATACTGAACATA |
| | | GAGCTGCTTCTGCTAGAGATTATAAAGATGATGATGAT |
| | | AAATGA |
| 388 | PpKRE2(M1- | ATGGTTCATATAGGTTTTAGAAGCTTGAAAGCCGTTTT |
| | D84)_Fam20c(R | CATTTTAGCATTGTCCTCATTGATTTTGTATGGTATTGT |
| | 64-R584)_FLAG | TACTACTTTTGATGGTTCTAGAGCTTCTAGATATCAAC |
| | | CACCATATGTTAATCATTCTCAAGATCCATTGTACCATT |
| | | CTGGTAACTCCTATAATAGAGAAAACGCGACTTTTGTT |
| | | ACCTTGTGTAGAAATGAAGATTTGTATTCTATTATCCA |
| | | ATCTATCAAGAAAGTCGAAGACAGAGGTAGACCTGGT |
| | | GAACCACCTGCTGCTTCGTCAGCTGCTGGTGATGCTGG |
| | | TTGGCCAAATAAACATACTTTGAGAATTTTGCAAGATT |
| | | TTTCTTCTGATCCATCTTCTAATTTATCTAGTCACTCTT |
| | | TGGAAAAATTGCCACCTGCTGCTGAGCCTGCTGAAAGA |
| | | GCTTTGAGAGGTAGAGACCCTGGTGCGCTCAGACCAC |
| | | ATGATCCTGCTCATAGACCATTGTTGAGAGACCCTGGT |
| | | CCTAGAAGATCTGAATCTCCACCTGGTCCTGGTGGTGA |
| | | TGCTTCTTTGTTGGCTAGATTGTTTGAACATCCATTGTA |
| | | TAGAGTTGCTGTTCCACCATTGACTGAAGAAGATGTTT |
| | | TGTTTAATGTTAATTCTGATACTAGATTGTCTCCAAAAG |
| | | CTGCTGAAAATCCTGATTGGCCACATGCTGGTGCTGAA |
| | | GGTGCTGAATTTTTGTCTCCTGGTGAAGCGGCTGTTGA |
| | | TTCCTATCCAAATTGGTTGAAATTCCACATCGGTATTA |
| | | ACCGATACGAATTGTATTCTAGACATAATCCTGCTATT |
| | | GAAGCTTTGTTGCATGATTTGTCTAGTCAGAGAATTAC |
| | | TTCTGTTGCTATGAAGTCTGGTGGTACTCAGTTGAAGT |
| | | TGATAATGACTTTTCAAAACTATGGGCAAGCTTTGTTT |
| | | AAACCAATGAAACAAACGAGAGAACAAGAAACTCCACC |
| | | TGATTTTTTTTATTTTTCGGATTATGAAAGACATAATGC |
| | | TGAAATTGCTGCTTTCCACTTGGACAGAATATTGGATT |
| | | TTCGCAGAGTTCCACCTGTTGCTGGTCGGATGGTTAAC |
| | | ATGACTAAGGAAATTAGAGATGTTACTAGAGATAAAAA |
| | | ATTGTGGAGAACTTTTTTCATTTCTCCGGCTAATAATAT |
| | | TTGTTTTTACGGGGAATGTTCTTATTATTGTTCTACTGA |
| | | ACATGCTTTGTGTGGTAAACCTGATCAAATTGAAGGTT |
| | | CTTTGGCTGCTTTTTTGCCTGATTTGTCTTTGGCTAAAA |
| | | GAAAAACTTGGAGAAATCCATGGAGAAGATCTTATCAT |
| | | AAAAGAAAAAAAGCTGAATGGGAAGTTGATCCTGATTA |
| | | TTGTGAAGAAGTTAAACAAACTCCACCATATGATTCGT |
| | | CTCATAGAATATTGGACGTCATGGATATGACGATCTTT |
| | | GACTTTCTGATGGGGAACATGGACAGACATCACTATGA |
| | | AACATTCGAAAAATTCGGTAATGAAACTTTTATCATCC |
| | | ATTTGGATAATGGTAGAGGTTTTGGTAAATATTCTCAT |
| | | GATGAATTGTCTATTTTGGTTCCATTGCAACAGTGTTG |
| | | TAGAATAAGGAAAAGCACTTACTTAAGATTACAACTCT |
| | | TGGCTAAAGAAGAATATAAATTGTCTTTGTTGATGGCT |
| | | GAATCTTTGAGAGGTGATCAAGTTGCTCCTGTTTTGTA |
| | | TCAACCACATTTGGAAGCTTTGGATAGAAGATTGAGAG |
| | | TTGTTTTGAAAGCTGTTAGAGATTGTGTTGAAAGAAAT |
| | | GGTTTGCATTCTGTTGTTGATGATGATTTGGATACTGA |
| | | ACATAGAGCTGCTTCTGCTAGAGATTATAAAGATGATG |
| | | ATGATAAATGA |
| 389 | PpKRE2(M1- | ATGGTTCATATAGGTTTTAGAAGCTTGAAAGCCGTTTT |
| | H150)_ | CATTTTAGCATTGTCCTCATTGATTTTGTATGGTATTGT |
| | Fam20c(R64- | TACTACTTTTGATGGTTCTAGAGCTTCTAGATATCAAC |
| | R584)_FLAG | CACCATATGTTAATCATTCTCAAGATCCATTGTACCATT |
| | | CTGGTAACTCCTATAATAGAGAAAACGCGACTTTTGTT |
| | | ACCTTGTGTAGAAATGAAGATTTGTATTCTATTATCCA |
| | | ATCTATCAAGAAAGTCGAAGACCGATTTAACAACAAAT |
| | | TTGCATACGATTGGGTTTTTCTGAATGAAGTTCCCTTT |
| | | ACTGATGAATTTAAAGAGAGGACTTCTGTTTTGATTTC |
| | | TGGTCAAGCTAAATATGGTTTGATTCCAAAAGAACATT |
| | | GGTCTTATCCTGATTATATTGATCAAGAAAGAGCTGCT |
| | | GAATCTAGAAGACAATTGGAAGATCAACATAGAGGTAG |
| | | ACCTGGTGAACCACCTGCTGCTTCGTCAGCTGCTGGTG |
| | | ATGCTGGTTGGCCAAATAAACATACTTTGAGAATTTTG |
| | | CAAGATTTTTCTTCTGATCCATCTTCTAATTTATCTAGT |
| | | CACTCTTTGGAAAAATTGCCACCTGCTGCTGAGCCTGC |
| | | TGAAAGAGCTTTGAGAGGTAGAGACCCTGGTGCGCTC |
| | | AGACCACATGATCCTGCTCATAGACCATTGTTGAGAGA |
| | | CCCTGGTCCTAGAAGATCTGAATCTCCACCTGGTCCTG |
| | | GTGGTGATGCTTCTTTGTTGGCTAGATTGTTTGAACAT |
| | | CCATTGTATAGAGTTGCTGTTCCACCATTGACTGAAGA |
| | | AGATGTTTTGTTTAATGTTAATTCTGATACTAGATTGTC |
| | | TCCAAAAGCTGCTGAAAATCCTGATTGGCCACATGCTG |
| | | GTGCTGAAGGTGCTGAATTTTTGTCTCCTGGTGAAGCG |
| | | GCTGTTGATTCCTATCCAAATTGGTTGAAATTCCACAT |
| | | CGGTATTAACCGATACGAATTGTATTCTAGACATAATC |
| | | CTGCTATTGAAGCTTTGTTGCATGATTTGTCTAGTCAG |
| | | AGAATTACTTCTGTTGCTATGAAGTCTGGTGGTACTCA |
| | | GTTGAAGTTGATAATGACTTTTCAAAACTATGGGCAAG |
| | | CTTTGTTTAAACCAATGAAACAAACGAGAGAACAAGAA |
| | | ACTCCACCTGATTTTTTTTATTTTTCGGATTATGAAAGA |
| | | CATAATGCTGAAATTGCTGCTTTCCACTTGGACAGAAT |
| | | ATTGGATTTTCGCAGAGTTCCACCTGTTGCTGGTCGGA |
| | | TGGTTAACATGACTAAGGAAATTAGAGATGTTACTAGA |
| | | GATAAAAAATTGTGGAGAACTTTTTTCATTTCTCCGGC |
| | | TAATAATATTTGTTTTTACGGGGAATGTTCTTATTATTG |
| | | TTCTACTGAACATGCTTTGTGTGGTAAACCTGATCAAA |
| | | TTGAAGGTTCTTTGGCTGCTTTTTTGCCTGATTTGTCTT |
| | | TGGCTAAAAGAAAAACTTGGAGAAATCCATGGAGAAG |
| | | ATCTTATCATAAAAGAAAAAAAGCTGAATGGGAAGTTG |
| | | ATCCTGATTATTGTGAAGAAGTTAAACAAACTCCACCA |
| | | TATGATTCGTCTCATAGAATATTGGACGTCATGGATAT |
| | | GACGATCTTTGACTTTCTGATGGGGAACATGGACAGAC |
| | | ATCACTATGAAACATTCGAAAAATTCGGTAATGAAACT |
| | | TTTATCATCCATTTGGATAATGGTAGAGGTTTTGGTAA |
| | | ATATTCTCATGATGAATTGTCTATTTTGGTTCCATTGCA |
| | | ACAGTGTTGTAGAATAAGGAAAAGCACTTACTTAAGAT |
| | | TACAACTCTTGGCTAAAGAAGAATATAAATTGTCTTTG |
| | | TTGATGGCTGAATCTTTGAGAGGTGATCAAGTTGCTCC |
| | | TGTTTTGTATCAACCACATTTGGAAGCTTTGGATAGAA |
| | | GATTGAGAGTTGTTTTGAAAGCTGTTAGAGATTGTGTT |
| | | GAAAGAAATGGTTTGCATTCTGTTGTTGATGATGATTT |
| | | GGATACTGAACATAGAGCTGCTTCTGCTAGAGATTATA |
| | | AAGATGATGATGATAAATGA |
| 390 | ScMNN2(M1- | ATGTTGCTAACTAAACGTTTCTCTAAGTTGTTCAAGTT |
| | S36)_Fam20c(R6 | GACGTTCATTGTTTTGATACTCTGTGGTTTGTTTGTTAT |
| | 4-R584)_FLAG | CACTAATAAATACATGGATGAAAATACTTCTAGAGGTA |
| | | GACCTGGTGAACCACCTGCTGCTTCGTCAGCTGCTGGT |
| | | GATGCTGGTTGGCCAAATAAACATACTTTGAGAATTTT |
| | | GCAAGATTTTTCTTCTGATCCATCTTCTAATTTATCTAG |
| | | TCACTCTTTGGAAAAATTGCCACCTGCTGCTGAGCCTG |
| | | CTGAAAGAGCTTTGAGAGGTAGAGACCCTGGTGCGCT |
| | | CAGACCACATGATCCTGCTCATAGACCATTGTTGAGAG |
| | | ACCCTGGTCCTAGAAGATCTGAATCTCCACCTGGTCCT |
| | | GGTGGTGATGCTTCTTTGTTGGCTAGATTGTTTGAACA |
| | | TCCATTGTATAGAGTTGCTGTTCCACCATTGACTGAAG |
| | | AAGATGTTTTGTTTAATGTTAATTCTGATACTAGATTGT |
| | | CTCCAAAAGCTGCTGAAAATCCTGATTGGCCACATGCT |
| | | GGTGCTGAAGGTGCTGAATTTTTGTCTCCTGGTGAAGC |
| | | GGCTGTTGATTCCTATCCAAATTGGTTGAAATTCCACA |
| | | TCGGTATTAACCGATACGAATTGTATTCTAGACATAAT |
| | | CCTGCTATTGAAGCTTTGTTGCATGATTIGTCTAGTCA |
| | | GAGAATTACTTCTGTTGCTATGAAGTCTGGTGGTACTC |
| | | AGTTGAAGTTGATAATGACTTTTCAAAACTATGGGCAA |
| | | GCTTTGTTTAAACCAATGAAACAAACGAGAGAACAAGA |
| | | AACTCCACCTGATTTTTTTTATTTTTCGGATTATGAAAG |
| | | ACATAATGCTGAAATTGCTGCTTTCCACTTGGACAGAA |
| | | TATTGGATTTTCGCAGAGTTCCACCTGTTGCTGGTCGG |
| | | ATGGTTAACATGACTAAGGAAATTAGAGATGTTACTAG |
| | | AGATAAAAAATTGTGGAGAACTTTTTTCATTTCTCCGG |
| | | CTAATAATATTTGTTTTTACGGGGAATGTTCTTATTATT |
| | | GTTCTACTGAACATGCTTTGTGTGGTAAACCTGATCAA |
| | | ATTGAAGGTTCTTTGGCTGCTTTTTTGCCTGATTTGTCT |
| | | TTGGCTAAAAGAAAAACTTGGAGAAATCCATGGAGAAG |
| | | ATCTTATCATAAAAGAAAAAAAGCTGAATGGGAAGTTG |
| | | ATCCTGATTATTGTGAAGAAGTTAAACAAACTCCACCA |
| | | TATGATTCGTCTCATAGAATATTGGACGTCATGGATAT |
| | | GACGATCTTTGACTTTCTGATGGGGAACATGGACAGAC |
| | | ATCACTATGAAACATTCGAAAAATTCGGTAATGAAACT |
| | | TTTATCATCCATTTGGATAATGGTAGAGGTTTTGGTAA |
| | | ATATTCTCATGATGAATTGTCTATTTTGGTTCCATTGCA |
| | | ACAGTGTTGTAGAATAAGGAAAAGCACTTACTTAAGAT |
| | | TACAACTCTTGGCTAAAGAAGAATATAAATTGTCTTTG |
| | | TTGATGGCTGAATCTTTGAGAGGTGATCAAGTTGCTCC |
| | | TGTTTTGTATCAACCACATTTGGAAGCTTTGGATAGAA |
| | | GATTGAGAGTTGTTTTGAAAGCTGTTAGAGATTGTGTT |
| | | GAAAGAAATGGTTTGCATTCTGTTGTTGATGATGATTT |
| | | GGATACTGAACATAGAGCTGCTTCTGCTAGAGATTATA |
| | | AAGATGATGATGATAAATGA |
| 391 | ScMNN2(M1- | ATGTTGCTAACTAAACGTTTCTCTAAGTTGTTCAAGTT |
| | P97)_Fam20c(R6 | GACGTTCATTGTTTTGATACTCTGTGGTTTGTTTGTTAT |
| | 4-R584)_FLAG | CACTAATAAATACATGGATGAAAATACTTCTGTAAAGG |
| | | AGTATAAAGAGTACCTAGACAGATATGTACAGTCTTAT |
| | | TCCAATAAATATTCTTCTTCTTCTGATGCTGCTTCTGCT |
| | | GATGATTCTACTCCCTTGCGAGACAATGATGAAGCCGG |
| | | TAACGAAAAACTCAAATCCTTTTACAATAATGTTTTTAA |
| | | CTTTTTGATGGTTGATTCACCAAGAGGTAGACCTGGTG |
| | | AACCACCTGCTGCTTCGTCAGCTGCTGGTGATGCTGGT |
| | | TGGCCAAATAAACATACTTTGAGAATTTTGCAAGATTT |
| | | TTCTTCTGATCCATCTTCTAATTTATCTAGTCACTCTTT |
| | | GGAAAAATTGCCACCTGCTGCTGAGCCTGCTGAAAGA |
| | | GCTTTGAGAGGTAGAGACCCTGGTGCGCTCAGACCAC |
| | | ATGATCCTGCTCATAGACCATTGTTGAGAGACCCTGGT |
| | | CCTAGAAGATCTGAATCTCCACCTGGTCCTGGTGGTGA |
| | | TGCTTCTTTGTTGGCTAGATTGTTTGAACATCCATTGTA |
| | | TAGAGTTGCTGTTCCACCATTGACTGAAGAAGATGTTT |
| | | TGTTTAATGTTAATTCTGATACTAGATTGTCTCCAAAAG |
| | | CTGCTGAAAATCCTGATTGGCCACATGCTGGTGCTGAA |
| | | GGTGCTGAATTTTTGTCTCCTGGTGAAGCGGCTGTTGA |
| | | TTCCTATCCAAATTGGTTGAAATTCCACATCGGTATTA |
| | | ACCGATACGAATTGTATTCTAGACATAATCCTGCTATT |
| | | GAAGCTTTGTTGCATGATTTGTCTAGTCAGAGAATTAC |
| | | TTCTGTTGCTATGAAGTCTGGTGGTACTCAGTTGAAGT |
| | | TGATAATGACTTTTCAAAACTATGGGCAAGCTTTGTTT |
| | | AAACCAATGAAACAAACGAGAGAACAAGAAACTCCACC |
| | | TGATTTTTTTTATTTTTCGGATTATGAAAGACATAATGC |
| | | TGAAATTGCTGCTTTCCACTTGGACAGAATATTGGATT |
| | | TTCGCAGAGTTCCACCTGTTGCTGGTCGGATGGTTAAC |
| | | ATGACTAAGGAAATTAGAGATGTTACTAGAGATAAAAA |
| | | ATTGTGGAGAACTTTTTTCATTTCTCCGGCTAATAATAT |
| | | TTGTTTTTACGGGGAATGTTCTTATTATTGTTCTACTGA |
| | | ACATGCTTTGTGTGGTAAACCTGATCAAATTGAAGGTT |
| | | CTTTGGCTGCTTTTTTGCCTGATTTGTCTTTGGCTAAAA |
| | | GAAAAACTTGGAGAAATCCATGGAGAAGATCTTATCAT |
| | | AAAAGAAAAAAAGCTGAATGGGAAGTTGATCCTGATTA |
| | | TTGTGAAGAAGTTAAACAAACTCCACCATATGATTCGT |
| | | CTCATAGAATATTGGACGTCATGGATATGACGATCTTT |
| | | GACTTTCTGATGGGGAACATGGACAGACATCACTATGA |
| | | AACATTCGAAAAATTCGGTAATGAAACTTTTATCATCC |
| | | ATTTGGATAATGGTAGAGGTTTTGGTAAATATTCTCAT |
| | | GATGAATTGTCTATTTTGGTTCCATTGCAACAGTGTTG |
| | | TAGAATAAGGAAAAGCACTTACTTAAGATTACAACTCT |
| | | TGGCTAAAGAAGAATATAAATTGTCTTTGTTGATGGCT |
| | | GAATCTTTGAGAGGTGATCAAGTTGCTCCTGTTTTGTA |
| | | TCAACCACATTTGGAAGCTTTGGATAGAAGATTGAGAG |
| | | TTGTTTTGAAAGCTGTTAGAGATTGTGTTGAAAGAAAT |
| | | GGTTTGCATTCTGTTGTTGATGATGATTTGGATACTGA |
| | | ACATAGAGCTGCTTCTGCTAGAGATTATAAAGATGATG |
| | | ATGATAAATGA |
| 392 | ScMNN2(M1- | ATGTTGCTAACTAAACGTTTCTCTAAGTTGTTCAAGTT |
| | S150)_Fam20c(R | GACGTTCATTGTTTTGATACTCTGTGGTTTGTTTGTTAT |
| | 64-R584)_FLAG | CACTAATAAATACATGGATGAAAATACTTCTGTAAAGG |
| | | AGTATAAAGAGTACCTAGACAGATATGTACAGTCTTAT |
| | | TCCAATAAATATTCTTCTTCTTCTGATGCTGCTTCTGCT |
| | | GATGATTCTACTCCCTTGCGAGACAATGATGAAGCCGG |
| | | TAACGAAAAACTCAAATCCTTTTACAATAATGTTTTTAA |
| | | CTTTTTGATGGTTGATTCACCAAAAGGTTCTACTGCTA |
| | | AACAATATAATGAAGCTTGTTTGTTGAAAGGTGATATT |
| | | GGAGATAGACCTGATCATTATAAGGATTTGTACAAATT |
| | | GTCTGCTAAAGAATTGTCTAAATGTTTGGAATTGTCTC |
| | | CTGATGAAGTTGCTTCTTTGACTAAATCTAGAGGTAGA |
| | | CCTGGTGAACCACCTGCTGCTTCGTCAGCTGCTGGTGA |
| | | TGCTGGTTGGCCAAATAAACATACTTTGAGAATTTTGC |
| | | AAGATTTTTCTTCTGATCCATCTTCTAATTTATCTAGTC |
| | | ACTCTTTGGAAAAATTGCCACCTGCTGCTGAGCCTGCT |
| | | GAAAGAGCTTTGAGAGGTAGAGACCCTGGTGCGCTCA |
| | | GACCACATGATCCTGCTCATAGACCATTGTTGAGAGAC |
| | | CCTGGTCCTAGAAGATCTGAATCTCCACCTGGTCCTGG |
| | | TGGTGATGCTTCTTTGTTGGCTAGATTGTTTGAACATC |
| | | CATTGTATAGAGTTGCTGTTCCACCATTGACTGAAGAA |
| | | GATGTTTTGTTTAATGTTAATTCTGATACTAGATTGTCT |
| | | CCAAAAGCTGCTGAAAATCCTGATTGGCCACATGCTGG |
| | | TGCTGAAGGTGCTGAATTTTTGTCTCCTGGTGAAGCGG |
| | | CTGTTGATTCCTATCCAAATTGGTTGAAATTCCACATC |
| | | GGTATTAACCGATACGAATTGTATTCTAGACATAATCC |
| | | TGCTATTGAAGCTTTGTTGCATGATTTGTCTAGTCAGA |
| | | GAATTACTTCTGTTGCTATGAAGTCTGGTGGTACTCAG |
| | | TTGAAGTTGATAATGACTTTTCAAAACTATGGGCAAGC |
| | | TTTGTTTAAACCAATGAAACAAACGAGAGAACAAGAAA |
| | | CTCCACCTGATTTTTTTTATTTTTCGGATTATGAAAGAC |
| | | ATAATGCTGAAATTGCTGCTTTCCACTTGGACAGAATA |
| | | TTGGATTTTCGCAGAGTTCCACCTGTTGCTGGTCGGAT |
| | | GGTTAACATGACTAAGGAAATTAGAGATGTTACTAGAG |
| | | ATAAAAAATTGTGGAGAACTTTTTTCATTTCTCCGGCT |
| | | AATAATATTTGTTTTTACGGGGAATGTTCTTATTATTGT |
| | | TCTACTGAACATGCTTTGTGTGGTAAACCTGATCAAAT |
| | | TGAAGGTTCTTTGGCTGCTTTTTTGCCTGATTTGTCTTT |
| | | GGCTAAAAGAAAAACTTGGAGAAATCCATGGAGAAGA |
| | | TCTTATCATAAAAGAAAAAAAGCTGAATGGGAAGTTGA |
| | | TCCTGATTATTGTGAAGAAGTTAAACAAACTCCACCAT |
| | | ATGATTCGTCTCATAGAATATTGGACGTCATGGATATG |
| | | ACGATCTTTGACTTTCTGATGGGGAACATGGACAGACA |
| | | TCACTATGAAACATTCGAAAAATTCGGTAATGAAACTT |
| | | TTATCATCCATTTGGATAATGGTAGAGGTTTTGGTAAA |
| | | TATTCTCATGATGAATTGTCTATTTTGGTTCCATTGCAA |
| | | CAGTGTTGTAGAATAAGGAAAAGCACTTACTTAAGATT |
| | | ACAACTCTTGGCTAAAGAAGAATATAAATTGTCTTTGT |
| | | TGATGGCTGAATCTTTGAGAGGTGATCAAGTTGCTCCT |
| | | GTTTTGTATCAACCACATTTGGAAGCTTTGGATAGAAG |
| | | ATTGAGAGTTGTTTTGAAAGCTGTTAGAGATTGTGTTG |
| | | AAAGAAATGGTTTGCATTCTGTTGTTGATGATGATTTG |
| | | GATACTGAACATAGAGCTGCTTCTGCTAGAGATTATAA |
| | | AGATGATGATGATAAATGA |
| 393 | ScMNN1(M1- | ATGTTGGCTTTGAGAAGATTTATCTTGAATCAGAGATC |
| | A42)_Fam20c(R | TTTGAGATCTTGTACTATACCAATATTGGTTGGGGCTT |
| | 64-R584)_FLAG | TGATTATTATTTTGGTTTTGTTTCAATTGGTCACTCATA |
| | | GAAACGATGCTAGAGGTAGACCTGGTGAACCACCTGC |
| | | TGCTTCGTCAGCTGCTGGTGATGCTGGTTGGCCAAATA |
| | | AACATACTTTGAGAATTTTGCAAGATTTTTCTTCTGATC |
| | | CATCTTCTAATTTATCTAGTCACTCTTTGGAAAAATTGC |
| | | CACCTGCTGCTGAGCCTGCTGAAAGAGCTTTGAGAGGT |
| | | AGAGACCCTGGTGCGCTCAGACCACATGATCCTGCTCA |
| | | TAGACCATTGTTGAGAGACCCTGGTCCTAGAAGATCTG |
| | | AATCTCCACCTGGTCCTGGTGGTGATGCTTCTTTGTTG |
| | | GCTAGATTGTTTGAACATCCATTGTATAGAGTTGCTGT |
| | | TCCACCATTGACTGAAGAAGATGTTTTGTTTAATGTTA |
| | | ATTCTGATACTAGATTGTCTCCAAAAGCTGCTGAAAAT |
| | | CCTGATTGGCCACATGCTGGTGCTGAAGGTGCTGAATT |
| | | TTTGTCTCCTGGTGAAGCGGCTGTTGATTCCTATCCAA |
| | | ATTGGTTGAAATTCCACATCGGTATTAACCGATACGAA |
| | | TTGTATTCTAGACATAATCCTGCTATTGAAGCTTTGTTG |
| | | CATGATTTGTCTAGTCAGAGAATTACTTCTGTTGCTAT |
| | | GAAGTCTGGTGGTACTCAGTTGAAGTTGATAATGACTT |
| | | TTCAAAACTATGGGCAAGCTTTGTTTAAACCAATGAAA |
| | | CAAACGAGAGAACAAGAAACTCCACCTGATTTTTTTTA |
| | | TTTTTCGGATTATGAAAGACATAATGCTGAAATTGCTG |
| | | CTTTCCACTTGGACAGAATATTGGATTTTCGCAGAGTT |
| | | CCACCTGTTGCTGGTCGGATGGTTAACATGACTAAGGA |
| | | AATTAGAGATGTTACTAGAGATAAAAAATTGTGGAGAA |
| | | CTTTTTTCATTTCTCCGGCTAATAATATTTGTTTTTACG |
| | | GGGAATGTTCTTATTATTGTTCTACTGAACATGCTTTGT |
| | | GTGGTAAACCTGATCAAATTGAAGGTTCTTTGGCTGCT |
| | | TTTTTGCCTGATTTGTCTTTGGCTAAAAGAAAAACTTG |
| | | GAGAAATCCATGGAGAAGATCTTATCATAAAAGAAAAA |
| | | AAGCTGAATGGGAAGTTGATCCTGATTATTGTGAAGAA |
| | | GTTAAACAAACTCCACCATATGATTCGTCTCATAGAAT |
| | | ATTGGACGTCATGGATATGACGATCTTTGACTTTCTGA |
| | | TGGGGAACATGGACAGACATCACTATGAAACATTCGAA |
| | | AAATTCGGTAATGAAACTTTTATCATCCATTTGGATAAT |
| | | GGTAGAGGTTTTGGTAAATATTCTCATGATGAATTGTC |
| | | TATTTTGGTTCCATTGCAACAGTGTTGTAGAATAAGGA |
| | | AAAGCACTTACTTAAGATTACAACTCTTGGCTAAAGAA |
| | | GAATATAAATTGTCTTTGTTGATGGCTGAATCTTTGAG |
| | | AGGTGATCAAGTTGCTCCTGTTTTGTATCAACCACATT |
| | | TGGAAGCTTTGGATAGAAGATTGAGAGTTGTTTTGAAA |
| | | GCTGTTAGAGATTGTGTTGAAAGAAATGGTTTGCATTC |
| | | TGTTGTTGATGATGATTTGGATACTGAACATAGAGCTG |
| | | CTTCTGCTAGAGATTATAAAGATGATGATGATAAATGA |
| 394 | ScMNN1(M1- | ATGTTGGCTTTGAGAAGATTTATCTTGAATCAGAGATC |
| | Q93)_Fam20c(R | TTTGAGATCTTGTACTATACCAATATTGGTTGGGGCTT |
| | 64-R584)_FLAG | TGATTATTATTTTGGTTTTGTTTCAATTGGTCACTCATA |
| | | GAAACGATGCTCTCATTAGATCCTCTAATGTTAATTCT |
| | | ACCAACAAAAAAACACTCAAGGATGCTGATCCAAAAGT |
| | | TTTGATTGAAGCTTTTGGTTCTCCTGAAGTTGATCCTG |
| | | TTGATACTATTCCTGTTTCTCCACTTGAATTGGTGCCAT |
| | | TTTACGATCAAAGAGGTAGACCTGGTGAACCACCTGCT |
| | | GCTTCGTCAGCTGCTGGTGATGCTGGTTGGCCAAATAA |
| | | ACATACTTTGAGAATTTTGCAAGATTTTTCTTCTGATCC |
| | | ATCTTCTAATTTATCTAGTCACTCTTTGGAAAAATTGCC |
| | | ACCTGCTGCTGAGCCTGCTGAAAGAGCTTTGAGAGGTA |
| | | GAGACCCTGGTGCGCTCAGACCACATGATCCTGCTCAT |
| | | AGACCATTGTTGAGAGACCCTGGTCCTAGAAGATCTGA |
| | | ATCTCCACCTGGTCCTGGTGGTGATGCTTCTTTGTTGG |
| | | CTAGATTGTTTGAACATCCATTGTATAGAGTTGCTGTT |
| | | CCACCATTGACTGAAGAAGATGTTTTGTTTAATGTTAA |
| | | TTCTGATACTAGATTGTCTCCAAAAGCTGCTGAAAATC |
| | | CTGATTGGCCACATGCTGGTGCTGAAGGTGCTGAATTT |
| | | TTGTCTCCTGGTGAAGCGGCTGTTGATTCCTATCCAAA |
| | | TTGGTTGAAATTCCACATCGGTATTAACCGATACGAAT |
| | | TGTATTCTAGACATAATCCTGCTATTGAAGCTTTGTTG |
| | | CATGATTTGTCTAGTCAGAGAATTACTTCTGTTGCTAT |
| | | GAAGTCTGGTGGTACTCAGTTGAAGTTGATAATGACTT |
| | | TTCAAAACTATGGGCAAGCTTTGTTTAAACCAATGAAA |
| | | CAAACGAGAGAACAAGAAACTCCACCTGATTTTTTTTA |
| | | TTTTTCGGATTATGAAAGACATAATGCTGAAATTGCTG |
| | | CTTTCCACTTGGACAGAATATTGGATTTTCGCAGAGTT |
| | | CCACCTGTTGCTGGTCGGATGGTTAACATGACTAAGGA |
| | | AATTAGAGATGTTACTAGAGATAAAAAATTGTGGAGAA |
| | | CTTTTTTCATTTCTCCGGCTAATAATATTTGTTTTTACG |
| | | GGGAATGTTCTTATTATTGTTCTACTGAACATGCTTTGT |
| | | GTGGTAAACCTGATCAAATTGAAGGTTCTTTGGCTGCT |
| | | TTTTTGCCTGATTTGTCTTTGGCTAAAAGAAAAACTTG |
| | | GAGAAATCCATGGAGAAGATCTTATCATAAAAGAAAAA |
| | | AAGCTGAATGGGAAGTTGATCCTGATTATTGTGAAGAA |
| | | GTTAAACAAACTCCACCATATGATTCGTCTCATAGAAT |
| | | ATTGGACGTCATGGATATGACGATCTTTGACTTTCTGA |
| | | TGGGGAACATGGACAGACATCACTATGAAACATTCGAA |
| | | AAATTCGGTAATGAAACTTTTATCATCCATTTGGATAAT |
| | | GGTAGAGGTTTTGGTAAATATTCTCATGATGAATTGTC |
| | | TATTTTGGTTCCATTGCAACAGTGTTGTAGAATAAGGA |
| | | AAAGCACTTACTTAAGATTACAACTCTTGGCTAAAGAA |
| | | GAATATAAATTGTCTTTGTTGATGGCTGAATCTTTGAG |
| | | AGGTGATCAAGTTGCTCCTGTTTTGTATCAACCACATT |
| | | TGGAAGCTTTGGATAGAAGATTGAGAGTTGTTTTGAAA |
| | | GCTGTTAGAGATTGTGTTGAAAGAAATGGTTTGCATTC |
| | | TGTTGTTGATGATGATTTGGATACTGAACATAGAGCTG |
| | | CTTCTGCTAGAGATTATAAAGATGATGATGATAAATGA |
| 395 | ScMNN1(M1- | ATGTTGGCTTTGAGAAGATTTATCTTGAATCAGAGATC |
| | G153)_Fam20c | TTTGAGATCTTGTACTATACCAATATTGGTTGGGGCTT |
| | (R64- | TGATTATTATTTTGGTTTTGTTTCAATTGGTCACTCATA |
| | R584)_FLAG | GAAACGATGCTCTCATTAGATCCTCTAATGTTAATTCT |
| | | ACCAACAAAAAAACACTCAAGGATGCTGATCCAAAAGT |
| | | TTTGATTGAAGCTTTTGGTTCTCCTGAAGTTGATCCTG |
| | | TTGATACTATTCCTGTTTCTCCACTTGAATTGGTGCCAT |
| | | TTTACGATCAATCTATTGATACTAAGAGGTCCTCTTCAT |
| | | GGTTGATAAACAAAAAGGGTTATTATAAACACTTCAAC |
| | | GAACTGTCTTTGACGGACAGATGCAAGTTCTATTTTAG |
| | | AACATTGTATACTCTAGACGATGAGTGGACTAACTCTG |
| | | TTAAAAAATTGGAATATTCAATTAATGATAATGAAGGT |
| | | AGAGGTAGACCTGGTGAACCACCTGCTGCTTCGTCAGC |
| | | TGCTGGTGATGCTGGTTGGCCAAATAAACATACTTTGA |
| | | GAATTTTGCAAGATTTTTCTTCTGATCCATCTTCTAATT |
| | | TATCTAGTCACTCTTTGGAAAAATTGCCACCTGCTGCT |
| | | GAGCCTGCTGAAAGAGCTTTGAGAGGTAGAGACCCTG |
| | | GTGCGCTCAGACCACATGATCCTGCTCATAGACCATTG |
| | | TTGAGAGACCCTGGTCCTAGAAGATCTGAATCTCCACC |
| | | TGGTCCTGGTGGTGATGCTTCTTTGTTGGCTAGATTGT |
| | | TTGAACATCCATTGTATAGAGTTGCTGTTCCACCATTG |
| | | ACTGAAGAAGATGTTTTGTTTAATGTTAATTCTGATACT |
| | | AGATTGTCTCCAAAAGCTGCTGAAAATCCTGATTGGCC |
| | | ACATGCTGGTGCTGAAGGTGCTGAATTTTTGTCTCCTG |
| | | GTGAAGCGGCTGTTGATTCCTATCCAAATTGGTTGAAA |
| | | TTCCACATCGGTATTAACCGATACGAATTGTATTCTAG |
| | | ACATAATCCTGCTATTGAAGCTTTGTTGCATGATTTGT |
| | | CTAGTCAGAGAATTACTTCTGTTGCTATGAAGTCTGGT |
| | | GGTACTCAGTTGAAGTTGATAATGACTTTTCAAAACTA |
| | | TGGGCAAGCTTTGTTTAAACCAATGAAACAAACGAGAG |
| | | AACAAGAAACTCCACCTGATTTTTTTTATTTTTCGGATT |
| | | ATGAAAGACATAATGCTGAAATTGCTGCTTTCCACTTG |
| | | GACAGAATATTGGATTTTCGCAGAGTTCCACCTGTTGC |
| | | TGGTCGGATGGTTAACATGACTAAGGAAATTAGAGATG |
| | | TTACTAGAGATAAAAAATTGTGGAGAACTTTTTTCATTT |
| | | CTCCGGCTAATAATATTTGTTTTTACGGGGAATGTTCT |
| | | TATTATTGTTCTACTGAACATGCTTTGTGTGGTAAACCT |
| | | GATCAAATTGAAGGTTCTTTGGCTGCTTTTTTGCCTGA |
| | | TTTGTCTTTGGCTAAAAGAAAAACTTGGAGAAATCCAT |
| | | GGAGAAGATCTTATCATAAAAGAAAAAAAGCTGAATGG |
| | | GAAGTTGATCCTGATTATTGTGAAGAAGTTAAACAAAC |
| | | TCCACCATATGATTCGTCTCATAGAATATTGGACGTCA |
| | | TGGATATGACGATCTTTGACTTTCTGATGGGGAACATG |
| | | GACAGACATCACTATGAAACATTCGAAAAATTCGGTAA |
| | | TGAAACTTTTATCATCCATTTGGATAATGGTAGAGGTT |
| | | TTGGTAAATATTCTCATGATGAATTGTCTATTTTGGTTC |
| | | CATTGCAACAGTGTTGTAGAATAAGGAAAAGCACTTAC |
| | | TTAAGATTACAACTCTTGGCTAAAGAAGAATATAAATT |
| | | GTCTTTGTTGATGGCTGAATCTTTGAGAGGTGATCAAG |
| | | TTGCTCCTGTTTTGTATCAACCACATTTGGAAGCTTTG |
| | | GATAGAAGATTGAGAGTTGTTTTGAAAGCTGTTAGAGA |
| | | TTGTGTTGAAAGAAATGGTTTGCATTCTGTTGTTGATG |
| | | ATGATTTGGATACTGAACATAGAGCTGCTTCTGCTAGA |
| | | GATTATAAAGATGATGATGATAAATGA |
| 396 | ScMNN6(M1- | ATGCATGTTTTGTTGTCTAAAAAGATTGCTAGATTTTTG |
| | P30)_Fam20c | TTGATATCTTTTGTTTTTGTTTTGGCTTTGATGGTTACC |
| | (R64-R584)_ | ATTAACCATCCGAGAGGTAGACCTGGTGAACCACCTGC |
| | FLAG | TGCTTCGTCAGCTGCTGGTGATGCTGGTTGGCCAAATA |
| | | AACATACTTTGAGAATTTTGCAAGATTTTTCTTCTGATC |
| | | CATCTTCTAATTTATCTAGTCACTCTTTGGAAAAATTGC |
| | | CACCTGCTGCTGAGCCTGCTGAAAGAGCTTTGAGAGGT |
| | | AGAGACCCTGGTGCGCTCAGACCACATGATCCTGCTCA |
| | | TAGACCATTGTTGAGAGACCCTGGTCCTAGAAGATCTG |
| | | AATCTCCACCTGGTCCTGGTGGTGATGCTTCTTTGTTG |
| | | GCTAGATTGTTTGAACATCCATTGTATAGAGTTGCTGT |
| | | TCCACCATTGACTGAAGAAGATGTTTTGTTTAATGTTA |
| | | ATTCTGATACTAGATTGTCTCCAAAAGCTGCTGAAAAT |
| | | CCTGATTGGCCACATGCTGGTGCTGAAGGTGCTGAATT |
| | | TTTGTCTCCTGGTGAAGCGGCTGTTGATTCCTATCCAA |
| | | ATTGGTTGAAATTCCACATCGGTATTAACCGATACGAA |
| | | TTGTATTCTAGACATAATCCTGCTATTGAAGCTTTGTTG |
| | | CATGATTTGTCTAGTCAGAGAATTACTTCTGTTGCTAT |
| | | GAAGTCTGGTGGTACTCAGTTGAAGTTGATAATGACTT |
| | | TTCAAAACTATGGGCAAGCTTTGTTTAAACCAATGAAA |
| | | CAAACGAGAGAACAAGAAACTCCACCTGATTTTTTTTA |
| | | TTTTTCGGATTATGAAAGACATAATGCTGAAATTGCTG |
| | | CTTTCCACTTGGACAGAATATTGGATTTTCGCAGAGTT |
| | | CCACCTGTTGCTGGTCGGATGGTTAACATGACTAAGGA |
| | | AATTAGAGATGTTACTAGAGATAAAAAATTGTGGAGAA |
| | | CTTTTTTCATTTCTCCGGCTAATAATATTTGTTTTTACG |
| | | GGGAATGTTCTTATTATTGTTCTACTGAACATGCTTTGT |
| | | GTGGTAAACCTGATCAAATTGAAGGTTCTTTGGCTGCT |
| | | TTTTTGCCTGATTTGTCTTTGGCTAAAAGAAAAACTTG |
| | | GAGAAATCCATGGAGAAGATCTTATCATAAAAGAAAAA |
| | | AAGCTGAATGGGAAGTTGATCCTGATTATTGTGAAGAA |
| | | GTTAAACAAACTCCACCATATGATTCGTCTCATAGAAT |
| | | ATTGGACGTCATGGATATGACGATCTTTGACTTTCTGA |
| | | TGGGGAACATGGACAGACATCACTATGAAACATTCGAA |
| | | AAATTCGGTAATGAAACTTTTATCATCCATTTGGATAAT |
| | | GGTAGAGGTTTTGGTAAATATTCTCATGATGAATTGTC |
| | | TATTTTGGTTCCATTGCAACAGTGTTGTAGAATAAGGA |
| | | AAAGCACTTACTTAAGATTACAACTCTTGGCTAAAGAA |
| | | GAATATAAATTGTCTTTGTTGATGGCTGAATCTTTGAG |
| | | AGGTGATCAAGTTGCTCCTGTTTTGTATCAACCACATT |
| | | TGGAAGCTTTGGATAGAAGATTGAGAGTTGTTTTGAAA |
| | | GCTGTTAGAGATTGTGTTGAAAGAAATGGTTTGCATTC |
| | | TGTTGTTGATGATGATTTGGATACTGAACATAGAGCTG |
| | | CTTCTGCTAGAGATTATAAAGATGATGATGATAAATGA |
| 397 | ScMNN6(M1- | ATGCATGTTTTGTTGTCTAAAAAGATTGCTAGATTTTTG |
| | V85)_Fam20c(R | TTGATATCTTTTGTTTTTGTTTTGGCTTTGATGGTTACC |
| | 64-R584)_FLAG | ATTAACCATCCGAAGACTAAACAAATGTCTGAACAATA |
| | | TGTTACTCCATATTTGCCAAAATCTTTGCAACCAATTGC |
| | | TAAAATTTCTGCTGAAGAACAAAGAAGAATTCAATCTG |
| | | AACAAGAAGAAGCTGAATTGAAACAATCTTTGGAAGGT |
| | | GAAGCAATAAGAAATGCTACCGTTAGAGGTAGACCTG |
| | | GTGAACCACCTGCTGCTTCGTCAGCTGCTGGTGATGCT |
| | | GGTTGGCCAAATAAACATACTTTGAGAATTTTGCAAGA |
| | | TTTTTCTTCTGATCCATCTTCTAATTTATCTAGTCACTC |
| | | TTTGGAAAAATTGCCACCTGCTGCTGAGCCTGCTGAAA |
| | | GAGCTTTGAGAGGTAGAGACCCTGGTGCGCTCAGACC |
| | | ACATGATCCTGCTCATAGACCATTGTTGAGAGACCCTG |
| | | GTCCTAGAAGATCTGAATCTCCACCTGGTCCTGGTGGT |
| | | GATGCTTCTTTGTTGGCTAGATTGTTTGAACATCCATT |
| | | GTATAGAGTTGCTGTTCCACCATTGACTGAAGAAGATG |
| | | TTTTGTTTAATGTTAATTCTGATACTAGATTGTCTCCAA |
| | | AAGCTGCTGAAAATCCTGATTGGCCACATGCTGGTGCT |
| | | GAAGGTGCTGAATTTTTGTCTCCTGGTGAAGCGGCTGT |
| | | TGATTCCTATCCAAATTGGTTGAAATTCCACATCGGTA |
| | | TTAACCGATACGAATTGTATTCTAGACATAATCCTGCT |
| | | ATTGAAGCTTTGTTGCATGATTTGTCTAGTCAGAGAAT |
| | | TACTTCTGTTGCTATGAAGTCTGGTGGTACTCAGTTGA |
| | | AGTTGATAATGACTTTTCAAAACTATGGGCAAGCTTTG |
| | | TTTAAACCAATGAAACAAACGAGAGAACAAGAAACTCC |
| | | ACCTGATTTTTTTTATTTTTCGGATTATGAAAGACATAA |
| | | TGCTGAAATTGCTGCTTTCCACTTGGACAGAATATTGG |
| | | ATTTTCGCAGAGTTCCACCTGTTGCTGGTCGGATGGTT |
| | | AACATGACTAAGGAAATTAGAGATGTTACTAGAGATAA |
| | | AAAATTGTGGAGAACTTTTTTCATTTCTCCGGCTAATA |
| | | ATATTTGTTTTTACGGGGAATGTTCTTATTATTGTTCTA |
| | | CTGAACATGCTTTGTGTGGTAAACCTGATCAAATTGAA |
| | | GGTTCTTTGGCTGCTTTTTTGCCTGATTTGTCTTTGGCT |
| | | AAAAGAAAAACTTGGAGAAATCCATGGAGAAGATCTTA |
| | | TCATAAAAGAAAAAAAGCTGAATGGGAAGTTGATCCTG |
| | | ATTATTGTGAAGAAGTTAAACAAACTCCACCATATGAT |
| | | TCGTCTCATAGAATATTGGACGTCATGGATATGACGAT |
| | | CTTTGACTTTCTGATGGGGAACATGGACAGACATCACT |
| | | ATGAAACATTCGAAAAATTCGGTAATGAAACTTTTATC |
| | | ATCCATTTGGATAATGGTAGAGGTTTTGGTAAATATTC |
| | | TCATGATGAATTGTCTATTTTGGTTCCATTGCAACAGT |
| | | GTTGTAGAATAAGGAAAAGCACTTACTTAAGATTACAA |
| | | CTCTTGGCTAAAGAAGAATATAAATTGTCTTTGTTGAT |
| | | GGCTGAATCTTTGAGAGGTGATCAAGTTGCTCCTGTTT |
| | | TGTATCAACCACATTTGGAAGCTTTGGATAGAAGATTG |
| | | AGAGTTGTTTTGAAAGCTGTTAGAGATTGTGTTGAAAG |
| | | AAATGGTTTGCATTCTGTTGTTGATGATGATTTGGATA |
| | | CTGAACATAGAGCTGCTTCTGCTAGAGATTATAAAGAT |
| | | GATGATGATAAATGA |
| 398 | ScMNN6(M1- | ATGCATGTTTTGTTGTCTAAAAAGATTGCTAGATTTTTG |
| | E160)_Fam20c(R | TTGATATCTTTTGTTTTTGTTTTGGCTTTGATGGTTACC |
| | 64-R584)_FLAG | ATTAACCATCCGAAGACTAAACAAATGTCTGAACAATA |
| | | TGTTACTCCATATTTGCCAAAATCTTTGCAACCAATTGC |
| | | TAAAATTTCTGCTGAAGAACAAAGAAGAATTCAATCTG |
| | | AACAAGAAGAAGCTGAATTGAAACAATCTTTGGAAGGT |
| | | GAAGCAATAAGAAATGCTACCGTTAACGCCATTAAAGA |
| | | AAAAATTAAATCTTATGGTGGTAATGAAACTACTTTGG |
| | | GTTTTATGGTTCCATCTTATATTAATCATAGAGGTTCTC |
| | | CACCCAAAGCTTGCTTCGTTTCATTGATCACAGAAAGG |
| | | GACTCTATGACTCAAATCTTGCAATCTATAGATGAGGT |
| | | CCAAGTCAAGTTTAACAAAAATTTTGCTTATCCATGGG |
| | | TTTTTATTTCTCAAGGTGAAAGAGGTAGACCTGGTGAA |
| | | CCACCTGCTGCTTCGTCAGCTGCTGGTGATGCTGGTTG |
| | | GCCAAATAAACATACTTTGAGAATTTTGCAAGATTTTT |
| | | CTTCTGATCCATCTTCTAATTTATCTAGTCACTCTTTGG |
| | | AAAAATTGCCACCTGCTGCTGAGCCTGCTGAAAGAGCT |
| | | TTGAGAGGTAGAGACCCTGGTGCGCTCAGACCACATG |
| | | ATCCTGCTCATAGACCATTGTTGAGAGACCCTGGTCCT |
| | | AGAAGATCTGAATCTCCACCTGGTCCTGGTGGTGATGC |
| | | TTCTTTGTTGGCTAGATTGTTTGAACATCCATTGTATAG |
| | | AGTTGCTGTTCCACCATTGACTGAAGAAGATGTTTTGT |
| | | TTAATGTTAATTCTGATACTAGATTGTCTCCAAAAGCT |
| | | GCTGAAAATCCTGATTGGCCACATGCTGGTGCTGAAGG |
| | | TGCTGAATTTTTGTCTCCTGGTGAAGCGGCTGTTGATT |
| | | CCTATCCAAATTGGTTGAAATTCCACATCGGTATTAAC |
| | | CGATACGAATTGTATTCTAGACATAATCCTGCTATTGA |
| | | AGCTTTGTTGCATGATTTGTCTAGTCAGAGAATTACTT |
| | | CTGTTGCTATGAAGTCTGGTGGTACTCAGTTGAAGTTG |
| | | ATAATGACTTTTCAAAACTATGGGCAAGCTTTGTTTAA |
| | | ACCAATGAAACAAACGAGAGAACAAGAAACTCCACCTG |
| | | ATTTTTTTTATTTTTCGGATTATGAAAGACATAATGCTG |
| | | AAATTGCTGCTTTCCACTTGGACAGAATATTGGATTTT |
| | | CGCAGAGTTCCACCTGTTGCTGGTCGGATGGTTAACAT |
| | | GACTAAGGAAATTAGAGATGTTACTAGAGATAAAAAAT |
| | | TGTGGAGAACTTTTTTCATTTCTCCGGCTAATAATATTT |
| | | GTTTTTACGGGGAATGTTCTTATTATTGTTCTACTGAAC |
| | | ATGCTTTGTGTGGTAAACCTGATCAAATTGAAGGTTCT |
| | | TTGGCTGCTTTTTTGCCTGATTTGTCTTTGGCTAAAAG |
| | | AAAAACTTGGAGAAATCCATGGAGAAGATCTTATCATA |
| | | AAAGAAAAAAAGCTGAATGGGAAGTTGATCCTGATTAT |
| | | TGTGAAGAAGTTAAACAAACTCCACCATATGATTCGTC |
| | | TCATAGAATATTGGACGTCATGGATATGACGATCTTTG |
| | | ACTTTCTGATGGGGAACATGGACAGACATCACTATGAA |
| | | ACATTCGAAAAATTCGGTAATGAAACTTTTATCATCCA |
| | | TTTGGATAATGGTAGAGGTTTTGGTAAATATTCTCATG |
| | | ATGAATTGTCTATTTTGGTTCCATTGCAACAGTGTTGT |
| | | AGAATAAGGAAAAGCACTTACTTAAGATTACAACTCTT |
| | | GGCTAAAGAAGAATATAAATTGTCTTTGTTGATGGCTG |
| | | AATCTTTGAGAGGTGATCAAGTTGCTCCTGTTTTGTAT |
| | | CAACCACATTTGGAAGCTTTGGATAGAAGATTGAGAGT |
| | | TGTTTTGAAAGCTGTTAGAGATTGTGTTGAAAGAAATG |
| | | GTTTGCATTCTGTTGTTGATGATGATTTGGATACTGAA |
| | | CATAGAGCTGCTTCTGCTAGAGATTATAAAGATGATGA |
| | | TGATAAATGA |
| 399 | Fam20c(D93- | ATGGATTTTTCTTCTGATCCATCTTCTAATTTATCTAGT |
| | R584)_FLAG | CACTCTTTGGAAAAATTGCCACCTGCTGCTGAGCCTGC |
| | | TGAAAGAGCTTTGAGAGGTAGAGACCCTGGTGCGCTC |
| | | AGACCACATGATCCTGCTCATAGACCATTGTTGAGAGA |
| | | CCCTGGTCCTAGAAGATCTGAATCTCCACCTGGTCCTG |
| | | GTGGTGATGCTTCTTTGTTGGCTAGATTGTTTGAACAT |
| | | CCATTGTATAGAGTTGCTGTTCCACCATTGACTGAAGA |
| | | AGATGTTTTGTTTAATGTTAATTCTGATACTAGATTGTC |
| | | TCCAAAAGCTGCTGAAAATCCTGATTGGCCACATGCTG |
| | | GTGCTGAAGGTGCTGAATTTTTGTCTCCTGGTGAAGCG |
| | | GCTGTTGATTCCTATCCAAATTGGTTGAAATTCCACAT |
| | | CGGTATTAACCGATACGAATTGTATTCTAGACATAATC |
| | | CTGCTATTGAAGCTTTGTTGCATGATTTGTCTAGTCAG |
| | | AGAATTACTTCTGTTGCTATGAAGTCTGGTGGTACTCA |
| | | GTTGAAGTTGATAATGACTTTTCAAAACTATGGGCAAG |
| | | CTTTGTTTAAACCAATGAAACAAACGAGAGAACAAGAA |
| | | ACTCCACCTGATTTTTTTTATTTTTCGGATTATGAAAGA |
| | | CATAATGCTGAAATTGCTGCTTTCCACTTGGACAGAAT |
| | | ATTGGATTTTCGCAGAGTTCCACCTGTTGCTGGTCGGA |
| | | TGGTTAACATGACTAAGGAAATTAGAGATGTTACTAGA |
| | | GATAAAAAATTGTGGAGAACTTTTTTCATTTCTCCGGC |
| | | TAATAATATTTGTTTTTACGGGGAATGTTCTTATTATTG |
| | | TTCTACTGAACATGCTTTGTGTGGTAAACCTGATCAAA |
| | | TTGAAGGTTCTTTGGCTGCTTTTTTGCCTGATTTGTCTT |
| | | TGGCTAAAAGAAAAACTTGGAGAAATCCATGGAGAAG |
| | | ATCTTATCATAAAAGAAAAAAAGCTGAATGGGAAGTTG |
| | | ATCCTGATTATTGTGAAGAAGTTAAACAAACTCCACCA |
| | | TATGATTCGTCTCATAGAATATTGGACGTCATGGATAT |
| | | GACGATCTTTGACTTTCTGATGGGGAACATGGACAGAC |
| | | ATCACTATGAAACATTCGAAAAATTCGGTAATGAAACT |
| | | TTTATCATCCATTTGGATAATGGTAGAGGTTTTGGTAA |
| | | ATATTCTCATGATGAATTGTCTATTTTGGTTCCATTGCA |
| | | ACAGTGTTGTAGAATAAGGAAAAGCACTTACTTAAGAT |
| | | TACAACTCTTGGCTAAAGAAGAATATAAATTGTCTTTG |
| | | TTGATGGCTGAATCTTTGAGAGGTGATCAAGTTGCTCC |
| | | TGTTTTGTATCAACCACATTTGGAAGCTTTGGATAGAA |
| | | GATTGAGAGTTGTTTTGAAAGCTGTTAGAGATTGTGTT |
| | | GAAAGAAATGGTTTGCATTCTGTTGTTGATGATGATTT |
| | | GGATACTGAACATAGAGCTGCTTCTGCTAGAGATTATA |
| | | AAGATGATGATGATAAATAA |
| 400 | ScKRE(M1- | ATGGCTTTATTTTTGTCTAAAAGATTGTTGAGATTTACT |
| | 158)_Fam20c(D9 | GTTATTGCTGGTGCTGTTATTGTTCTATTGTTGACGCT |
| | 3-R584)_FLAG | CAATTCAAATTCACGAACTCAACAATATATTCCATCTTC |
| | | TATTTCTGCTGCTTTTGATTTTACTTCTGGTTCTATTTC |
| | | TCCTGAACAACAAGTTATTGATTTTTCTTCTGATCCATC |
| | | TTCTAATTTATCTAGTCACTCTTTGGAAAAATTGCCACC |
| | | TGCTGCTGAGCCTGCTGAAAGAGCTTTGAGAGGTAGA |
| | | GACCCTGGTGCGCTCAGACCACATGATCCTGCTCATAG |
| | | ACCATTGTTGAGAGACCCTGGTCCTAGAAGATCTGAAT |
| | | CTCCACCTGGTCCTGGTGGTGATGCTTCTTTGTTGGCT |
| | | AGATTGTTTGAACATCCATTGTATAGAGTTGCTGTTCC |
| | | ACCATTGACTGAAGAAGATGTTTTGTTTAATGTTAATT |
| | | CTGATACTAGATTGTCTCCAAAAGCTGCTGAAAATCCT |
| | | GATTGGCCACATGCTGGTGCTGAAGGTGCTGAATTTTT |
| | | GTCTCCTGGTGAAGCGGCTGTTGATTCCTATCCAAATT |
| | | GGTTGAAATTCCACATCGGTATTAACCGATACGAATTG |
| | | TATTCTAGACATAATCCTGCTATTGAAGCTTTGTTGCAT |
| | | GATTTGTCTAGTCAGAGAATTACTTCTGTTGCTATGAA |
| | | GTCTGGTGGTACTCAGTTGAAGTTGATAATGACTTTTC |
| | | AAAACTATGGGCAAGCTTTGTTTAAACCAATGAAACAA |
| | | ACGAGAGAACAAGAAACTCCACCTGATTTTTTTTATTT |
| | | TTCGGATTATGAAAGACATAATGCTGAAATTGCTGCTT |
| | | TCCACTTGGACAGAATATTGGATTTTCGCAGAGTTCCA |
| | | CCTGTTGCTGGTCGGATGGTTAACATGACTAAGGAAAT |
| | | TAGAGATGTTACTAGAGATAAAAAATTGTGGAGAACTT |
| | | TTTTCATTTCTCCGGCTAATAATATTTGTTTTTACGGGG |
| | | AATGTTCTTATTATTGTTCTACTGAACATGCTTTGTGTG |
| | | GTAAACCTGATCAAATTGAAGGTTCTTTGGCTGCTTTT |
| | | TTGCCTGATTTGTCTTTGGCTAAAAGAAAAACTTGGAG |
| | | AAATCCATGGAGAAGATCTTATCATAAAAGAAAAAAAG |
| | | CTGAATGGGAAGTTGATCCTGATTATTGTGAAGAAGTT |
| | | AAACAAACTCCACCATATGATTCGTCTCATAGAATATT |
| | | GGACGTCATGGATATGACGATCTTTGACTTTCTGATGG |
| | | GGAACATGGACAGACATCACTATGAAACATTCGAAAAA |
| | | TTCGGTAATGAAACTTTTATCATCCATTTGGATAATGG |
| | | TAGAGGTTTTGGTAAATATTCTCATGATGAATTGTCTA |
| | | TTTTGGTTCCATTGCAACAGTGTTGTAGAATAAGGAAA |
| | | AGCACTTACTTAAGATTACAACTCTTGGCTAAAGAAGA |
| | | ATATAAATTGTCTTTGTTGATGGCTGAATCTTTGAGAG |
| | | GTGATCAAGTTGCTCCTGTTTTGTATCAACCACATTTG |
| | | GAAGCTTTGGATAGAAGATTGAGAGTTGTTTTGAAAGC |
| | | TGTTAGAGATTGTGTTGAAAGAAATGGTTTGCATTCTG |
| | | TTGTTGATGATGATTTGGATACTGAACATAGAGCTGCT |
| | | TCTGCTAGAGATTATAAAGATGATGATGATAAATAA |
| 401 | ScKRE2(M1- | ATGGCTTTATTTTTGTCTAAAAGATTGTTGAGATTTACT |
| | S80)_Fam20c(D9 | GTTATTGCTGGTGCTGTTATTGTTCTATTGTTGACGCT |
| | 3-R584)_FLAG | CAATTCAAATTCACGAACTCAACAATATATTCCATCTTC |
| | | TATTTCTGCTGCTTTTGATTTTACTTCTGGTTCTATTTC |
| | | TCCTGAACAACAAGTTATTTCTGAAGAAAATGATGCTA |
| | | AGAAATTAGAACAGTCCGCTTTGAATTCTGAAGCTTCT |
| | | GAAGATTCTGATTTTTCTTCTGATCCATCTTCTAATTTA |
| | | TCTAGTCACTCTTTGGAAAAATTGCCACCTGCTGCTGA |
| | | GCCTGCTGAAAGAGCTTTGAGAGGTAGAGACCCTGGT |
| | | GCGCTCAGACCACATGATCCTGCTCATAGACCATTGTT |
| | | GAGAGACCCTGGTCCTAGAAGATCTGAATCTCCACCTG |
| | | GTCCTGGTGGTGATGCTTCTTTGTTGGCTAGATTGTTT |
| | | GAACATCCATTGTATAGAGTTGCTGTTCCACCATTGAC |
| | | TGAAGAAGATGTTTTGTTTAATGTTAATTCTGATACTA |
| | | GATTGTCTCCAAAAGCTGCTGAAAATCCTGATTGGCCA |
| | | CATGCTGGTGCTGAAGGTGCTGAATTTTTGTCTCCTGG |
| | | TGAAGCGGCTGTTGATTCCTATCCAAATTGGTTGAAAT |
| | | TCCACATCGGTATTAACCGATACGAATTGTATTCTAGA |
| | | CATAATCCTGCTATTGAAGCTTTGTTGCATGATTTGTCT |
| | | AGTCAGAGAATTACTTCTGTTGCTATGAAGTCTGGTGG |
| | | TACTCAGTTGAAGTTGATAATGACTTTTCAAAACTATG |
| | | GGCAAGCTTTGTTTAAACCAATGAAACAAACGAGAGAA |
| | | CAAGAAACTCCACCTGATTTTTTTTATTTTTCGGATTAT |
| | | GAAAGACATAATGCTGAAATTGCTGCTTTCCACTTGGA |
| | | CAGAATATTGGATTTTCGCAGAGTTCCACCTGTTGCTG |
| | | GTCGGATGGTTAACATGACTAAGGAAATTAGAGATGTT |
| | | ACTAGAGATAAAAAATTGTGGAGAACTTTTTTCATTTC |
| | | TCCGGCTAATAATATTTGTTTTTACGGGGAATGTTCTT |
| | | ATTATTGTTCTACTGAACATGCTTTGTGTGGTAAACCT |
| | | GATCAAATTGAAGGTTCTTTGGCTGCTTTTTTGCCTGA |
| | | TTTGTCTTTGGCTAAAAGAAAAACTTGGAGAAATCCAT |
| | | GGAGAAGATCTTATCATAAAAGAAAAAAAGCTGAATGG |
| | | GAAGTTGATCCTGATTATTGTGAAGAAGTTAAACAAAC |
| | | TCCACCATATGATTCGTCTCATAGAATATTGGACGTCA |
| | | TGGATATGACGATCTTTGACTTTCTGATGGGGAACATG |
| | | GACAGACATCACTATGAAACATTCGAAAAATTCGGTAA |
| | | TGAAACTTTTATCATCCATTTGGATAATGGTAGAGGTT |
| | | TTGGTAAATATTCTCATGATGAATTGTCTATTTTGGTTC |
| | | CATTGCAACAGTGTTGTAGAATAAGGAAAAGCACTTAC |
| | | TTAAGATTACAACTCTTGGCTAAAGAAGAATATAAATT |
| | | GTCTTTGTTGATGGCTGAATCTTTGAGAGGTGATCAAG |
| | | TTGCTCCTGTTTTGTATCAACCACATTTGGAAGCTTTG |
| | | GATAGAAGATTGAGAGTTGTTTTGAAAGCTGTTAGAGA |
| | | TTGTGTTGAAAGAAATGGTTTGCATTCTGTTGTTGATG |
| | | ATGATTTGGATACTGAACATAGAGCTGCTTCTGCTAGA |
| | | GATTATAAAGATGATGATGATAAATAA |
| 402 | ScKRE2(M1- | ATGGCTTTATTTTTGTCTAAAAGATTGTTGAGATTTACT |
| | D102)_Fam20c | GTTATTGCTGGTGCTGTTATTGTTCTATTGTTGACGCT |
| | (D93- | CAATTCAAATTCACGAACTCAACAATATATTCCATCTTC |
| | R584)_FLAG | TATTTCTGCTGCTTTTGATTTTACTTCTGGTTCTATTTC |
| | | TCCTGAACAACAAGTTATTTCTGAAGAAAATGATGCTA |
| | | AGAAATTAGAACAGTCCGCTTTGAATTCTGAAGCTTCT |
| | | GAAGATTCTGAAGCTATGGATGAAGAATCTAAAGCTTT |
| | | GAAAGCTGCTGCTGAAAAAGCTGATGCTCCAATTGATG |
| | | ATTTTTCTTCTGATCCATCTTCTAATTTATCTAGTCACT |
| | | CTTTGGAAAAATTGCCACCTGCTGCTGAGCCTGCTGAA |
| | | AGAGCTTTGAGAGGTAGAGACCCTGGTGCGCTCAGAC |
| | | CACATGATCCTGCTCATAGACCATTGTTGAGAGACCCT |
| | | GGTCCTAGAAGATCTGAATCTCCACCTGGTCCTGGTGG |
| | | TGATGCTTCTTTGTTGGCTAGATTGTTTGAACATCCATT |
| | | GTATAGAGTTGCTGTTCCACCATTGACTGAAGAAGATG |
| | | TTTTGTTTAATGTTAATTCTGATACTAGATTGTCTCCAA |
| | | AAGCTGCTGAAAATCCTGATTGGCCACATGCTGGTGCT |
| | | GAAGGTGCTGAATTTTTGTCTCCTGGTGAAGCGGCTGT |
| | | TGATTCCTATCCAAATTGGTTGAAATTCCACATCGGTA |
| | | TTAACCGATACGAATTGTATTCTAGACATAATCCTGCT |
| | | ATTGAAGCTTTGTTGCATGATTTGTCTAGTCAGAGAAT |
| | | TACTTCTGTTGCTATGAAGTCTGGTGGTACTCAGTTGA |
| | | AGTTGATAATGACTTTTCAAAACTATGGGCAAGCTTTG |
| | | TTTAAACCAATGAAACAAACGAGAGAACAAGAAACTCC |
| | | ACCTGATTTTTTTTATTTTTCGGATTATGAAAGACATAA |
| | | TGCTGAAATTGCTGCTTTCCACTTGGACAGAATATTGG |
| | | ATTTTCGCAGAGTTCCACCTGTTGCTGGTCGGATGGTT |
| | | AACATGACTAAGGAAATTAGAGATGTTACTAGAGATAA |
| | | AAAATTGTGGAGAACTTTTTTCATTTCTCCGGCTAATA |
| | | ATATTTGTTTTTACGGGGAATGTTCTTATTATTGTTCTA |
| | | CTGAACATGCTTTGTGTGGTAAACCTGATCAAATTGAA |
| | | GGTTCTTTGGCTGCTTTTTTGCCTGATTTGTCTTTGGCT |
| | | AAAAGAAAAACTTGGAGAAATCCATGGAGAAGATCTTA |
| | | TCATAAAAGAAAAAAAGCTGAATGGGAAGTTGATCCTG |
| | | ATTATTGTGAAGAAGTTAAACAAACTCCACCATATGAT |
| | | TCGTCTCATAGAATATTGGACGTCATGGATATGACGAT |
| | | CTTTGACTTTCTGATGGGGAACATGGACAGACATCACT |
| | | ATGAAACATTCGAAAAATTCGGTAATGAAACTTTTATC |
| | | ATCCATTTGGATAATGGTAGAGGTTTTGGTAAATATTC |
| | | TCATGATGAATTGTCTATTTTGGTTCCATTGCAACAGT |
| | | GTTGTAGAATAAGGAAAAGCACTTACTTAAGATTACAA |
| | | CTCTTGGCTAAAGAAGAATATAAATTGTCTTTGTTGAT |
| | | GGCTGAATCTTTGAGAGGTGATCAAGTTGCTCCTGTTT |
| | | TGTATCAACCACATTTGGAAGCTTTGGATAGAAGATTG |
| | | AGAGTTGTTTTGAAAGCTGTTAGAGATTGTGTTGAAAG |
| | | AAATGGTTTGCATTCTGTTGTTGATGATGATTTGGATA |
| | | CTGAACATAGAGCTGCTTCTGCTAGAGATTATAAAGAT |
| | | GATGATGATAAATAA |
| 403 | PpKRE2(M1- | ATGGTTCATATAGGTTTTAGAAGCTTGAAAGCCGTTTT |
| | G31)_Fam20c(D | CATTTTAGCATTGTCCTCATTGATTTTGTATGGTATTGT |
| | 93-R584)_FLAG | TACTACTTTTGATGGTGATTTTTCTTCTGATCCATCTTC |
| | | TAATTTATCTAGTCACTCTTTGGAAAAATTGCCACCTG |
| | | CTGCTGAGCCTGCTGAAAGAGCTTTGAGAGGTAGAGA |
| | | CCCTGGTGCGCTCAGACCACATGATCCTGCTCATAGAC |
| | | CATTGTTGAGAGACCCTGGTCCTAGAAGATCTGAATCT |
| | | CCACCTGGTCCTGGTGGTGATGCTTCTTTGTTGGCTAG |
| | | ATTGTTTGAACATCCATTGTATAGAGTTGCTGTTCCAC |
| | | CATTGACTGAAGAAGATGTTTTGTTTAATGTTAATTCT |
| | | GATACTAGATTGTCTCCAAAAGCTGCTGAAAATCCTGA |
| | | TTGGCCACATGCTGGTGCTGAAGGTGCTGAATTTTTGT |
| | | CTCCTGGTGAAGCGGCTGTTGATTCCTATCCAAATTGG |
| | | TTGAAATTCCACATCGGTATTAACCGATACGAATTGTA |
| | | TTCTAGACATAATCCTGCTATTGAAGCTTTGTTGCATG |
| | | ATTTGTCTAGTCAGAGAATTACTTCTGTTGCTATGAAG |
| | | TCTGGTGGTACTCAGTTGAAGTTGATAATGACTTTTCA |
| | | AAACTATGGGCAAGCTTTGTTTAAACCAATGAAACAAA |
| | | CGAGAGAACAAGAAACTCCACCTGATTTTTTTTATTTTT |
| | | CGGATTATGAAAGACATAATGCTGAAATTGCTGCTTTC |
| | | CACTTGGACAGAATATTGGATTTTCGCAGAGTTCCACC |
| | | TGTTGCTGGTCGGATGGTTAACATGACTAAGGAAATTA |
| | | GAGATGTTACTAGAGATAAAAAATTGTGGAGAACTTTT |
| | | TTCATTTCTCCGGCTAATAATATTTGTTTTTACGGGGAA |
| | | TGTTCTTATTATTGTTCTACTGAACATGCTTTGTGTGGT |
| | | AAACCTGATCAAATTGAAGGTTCTTTGGCTGCTTTTTT |
| | | GCCTGATTTGTCTTTGGCTAAAAGAAAAACTTGGAGAA |
| | | ATCCATGGAGAAGATCTTATCATAAAAGAAAAAAAGCT |
| | | GAATGGGAAGTTGATCCTGATTATTGTGAAGAAGTTAA |
| | | ACAAACTCCACCATATGATTCGTCTCATAGAATATTGG |
| | | ACGTCATGGATATGACGATCTTTGACTTTCTGATGGGG |
| | | AACATGGACAGACATCACTATGAAACATTCGAAAAATT |
| | | CGGTAATGAAACTTTTATCATCCATTTGGATAATGGTA |
| | | GAGGTTTTGGTAAATATTCTCATGATGAATTGTCTATTT |
| | | TGGTTCCATTGCAACAGTGTTGTAGAATAAGGAAAAGC |
| | | ACTTACTTAAGATTACAACTCTTGGCTAAAGAAGAATA |
| | | TAAATTGTCTTTGTTGATGGCTGAATCTTTGAGAGGTG |
| | | ATCAAGTTGCTCCTGTTTTGTATCAACCACATTTGGAA |
| | | GCTTTGGATAGAAGATTGAGAGTTGTTTTGAAAGCTGT |
| | | TAGAGATTGTGTTGAAAGAAATGGTTTGCATTCTGTTG |
| | | TTGATGATGATTTGGATACTGAACATAGAGCTGCTTCT |
| | | GCTAGAGATTATAAAGATGATGATGATAAATAA |
| 404 | PpKRE2(M1- | ATGGTTCATATAGGTTTTAGAAGCTTGAAAGCCGTTTT |
| | D84)_Fam20c(D | CATTTTAGCATTGTCCTCATTGATTTTGTATGGTATTGT |
| | 93-R584)_FLAG | TACTACTTTTGATGGTTCTAGAGCTTCTAGATATCAAC |
| | | CACCATATGTTAATCATTCTCAAGATCCATTGTACCATT |
| | | CTGGTAACTCCTATAATAGAGAAAACGCGACTTTTGTT |
| | | ACCTTGTGTAGAAATGAAGATTTGTATTCTATTATCCA |
| | | ATCTATCAAGAAAGTCGAAGACGATTTTTCTTCTGATC |
| | | CATCTTCTAATTTATCTAGTCACTCTTTGGAAAAATTGC |
| | | CACCTGCTGCTGAGCCTGCTGAAAGAGCTTTGAGAGGT |
| | | AGAGACCCTGGTGCGCTCAGACCACATGATCCTGCTCA |
| | | TAGACCATTGTTGAGAGACCCTGGTCCTAGAAGATCTG |
| | | AATCTCCACCTGGTCCTGGTGGTGATGCTTCTTTGTTG |
| | | GCTAGATTGTTTGAACATCCATTGTATAGAGTTGCTGT |
| | | TCCACCATTGACTGAAGAAGATGTTTTGTTTAATGTTA |
| | | ATTCTGATACTAGATTGTCTCCAAAAGCTGCTGAAAAT |
| | | CCTGATTGGCCACATGCTGGTGCTGAAGGTGCTGAATT |
| | | TTTGTCTCCTGGTGAAGCGGCTGTTGATTCCTATCCAA |
| | | ATTGGTTGAAATTCCACATCGGTATTAACCGATACGAA |
| | | TTGTATTCTAGACATAATCCTGCTATTGAAGCTTTGTTG |
| | | CATGATTTGTCTAGTCAGAGAATTACTTCTGTTGCTAT |
| | | GAAGTCTGGTGGTACTCAGTTGAAGTTGATAATGACTT |
| | | TTCAAAACTATGGGCAAGCTTTGTTTAAACCAATGAAA |
| | | CAAACGAGAGAACAAGAAACTCCACCTGATTTTTTTTA |
| | | TTTTTCGGATTATGAAAGACATAATGCTGAAATTGCTG |
| | | CTTTCCACTTGGACAGAATATTGGATTTTCGCAGAGTT |
| | | CCACCTGTTGCTGGTCGGATGGTTAACATGACTAAGGA |
| | | AATTAGAGATGTTACTAGAGATAAAAAATTGTGGAGAA |
| | | CTTTTTTCATTTCTCCGGCTAATAATATTTGTTTTTACG |
| | | GGGAATGTTCTTATTATTGTTCTACTGAACATGCTTTGT |
| | | GTGGTAAACCTGATCAAATTGAAGGTTCTTTGGCTGCT |
| | | TTTTTGCCTGATTTGTCTTTGGCTAAAAGAAAAACTTG |
| | | GAGAAATCCATGGAGAAGATCTTATCATAAAAGAAAAA |
| | | AAGCTGAATGGGAAGTTGATCCTGATTATTGTGAAGAA |
| | | GTTAAACAAACTCCACCATATGATTCGTCTCATAGAAT |
| | | ATTGGACGTCATGGATATGACGATCTTTGACTTTCTGA |
| | | TGGGGAACATGGACAGACATCACTATGAAACATTCGAA |
| | | AAATTCGGTAATGAAACTTTTATCATCCATTTGGATAAT |
| | | GGTAGAGGTTTTGGTAAATATTCTCATGATGAATTGTC |
| | | TATTTTGGTTCCATTGCAACAGTGTTGTAGAATAAGGA |
| | | AAAGCACTTACTTAAGATTACAACTCTTGGCTAAAGAA |
| | | GAATATAAATTGTCTTTGTTGATGGCTGAATCTTTGAG |
| | | AGGTGATCAAGTTGCTCCTGTTTTGTATCAACCACATT |
| | | TGGAAGCTTTGGATAGAAGATTGAGAGTTGTTTTGAAA |
| | | GCTGTTAGAGATTGTGTTGAAAGAAATGGTTTGCATTC |
| | | TGTTGTTGATGATGATTTGGATACTGAACATAGAGCTG |
| | | CTTCTGCTAGAGATTATAAAGATGATGATGATAAATAA |
| 405 | PpKRE2(M1- | ATGGTTCATATAGGTTTTAGAAGCTTGAAAGCCGTTTT |
| | H150)_Fam20c( | CATTTTAGCATTGTCCTCATTGATTTTGTATGGTATTGT |
| | D93- | TACTACTTTTGATGGTTCTAGAGCTTCTAGATATCAAC |
| | R584)_FLAG | CACCATATGTTAATCATTCTCAAGATCCATTGTACCATT |
| | | CTGGTAACTCCTATAATAGAGAAAACGCGACTTTTGTT |
| | | ACCTTGTGTAGAAATGAAGATTTGTATTCTATTATCCA |
| | | ATCTATCAAGAAAGTCGAAGACCGATTTAACAACAAAT |
| | | TTGCATACGATTGGGTTTTTCTGAATGAAGTTCCCTTT |
| | | ACTGATGAATTTAAAGAGAGGACTTCTGTTTTGATTTC |
| | | TGGTCAAGCTAAATATGGTTTGATTCCAAAAGAACATT |
| | | GGTCTTATCCTGATTATATTGATCAAGAAAGAGCTGCT |
| | | GAATCTAGAAGACAATTGGAAGATCAACATGATTTTTC |
| | | TTCTGATCCATCTTCTAATTTATCTAGTCACTCTTTGGA |
| | | AAAATTGCCACCTGCTGCTGAGCCTGCTGAAAGAGCTT |
| | | TGAGAGGTAGAGACCCTGGTGCGCTCAGACCACATGA |
| | | TCCTGCTCATAGACCATTGTTGAGAGACCCTGGTCCTA |
| | | GAAGATCTGAATCTCCACCTGGTCCTGGTGGTGATGCT |
| | | TCTTTGTTGGCTAGATTGTTTGAACATCCATTGTATAG |
| | | AGTTGCTGTTCCACCATTGACTGAAGAAGATGTTTTGT |
| | | TTAATGTTAATTCTGATACTAGATTGTCTCCAAAAGCT |
| | | GCTGAAAATCCTGATTGGCCACATGCTGGTGCTGAAGG |
| | | TGCTGAATTTTTGTCTCCTGGTGAAGCGGCTGTTGATT |
| | | CCTATCCAAATTGGTTGAAATTCCACATCGGTATTAAC |
| | | CGATACGAATTGTATTCTAGACATAATCCTGCTATTGA |
| | | AGCTTTGTTGCATGATTTGTCTAGTCAGAGAATTACTT |
| | | CTGTTGCTATGAAGTCTGGTGGTACTCAGTTGAAGTTG |
| | | ATAATGACTTTTCAAAACTATGGGCAAGCTTTGTTTAA |
| | | ACCAATGAAACAAACGAGAGAACAAGAAACTCCACCTG |
| | | ATTTTTTTTATTTTTCGGATTATGAAAGACATAATGCTG |
| | | AAATTGCTGCTTTCCACTTGGACAGAATATTGGATTTT |
| | | CGCAGAGTTCCACCTGTTGCTGGTCGGATGGTTAACAT |
| | | GACTAAGGAAATTAGAGATGTTACTAGAGATAAAAAAT |
| | | TGTGGAGAACTTTTTTCATTTCTCCGGCTAATAATATTT |
| | | GTTTTTACGGGGAATGTTCTTATTATTGTTCTACTGAAC |
| | | ATGCTTTGTGTGGTAAACCTGATCAAATTGAAGGTTCT |
| | | TTGGCTGCTTTTTTGCCTGATTTGTCTTTGGCTAAAAG |
| | | AAAAACTTGGAGAAATCCATGGAGAAGATCTTATCATA |
| | | AAAGAAAAAAAGCTGAATGGGAAGTTGATCCTGATTAT |
| | | TGTGAAGAAGTTAAACAAACTCCACCATATGATTCGTC |
| | | TCATAGAATATTGGACGTCATGGATATGACGATCTTTG |
| | | ACTTTCTGATGGGGAACATGGACAGACATCACTATGAA |
| | | ACATTCGAAAAATTCGGTAATGAAACTTTTATCATCCA |
| | | TTTGGATAATGGTAGAGGTTTTGGTAAATATTCTCATG |
| | | ATGAATTGTCTATTTTGGTTCCATTGCAACAGTGTTGT |
| | | AGAATAAGGAAAAGCACTTACTTAAGATTACAACTCTT |
| | | GGCTAAAGAAGAATATAAATTGTCTTTGTTGATGGCTG |
| | | AATCTTTGAGAGGTGATCAAGTTGCTCCTGTTTTGTAT |
| | | CAACCACATTTGGAAGCTTTGGATAGAAGATTGAGAGT |
| | | TGTTTTGAAAGCTGTTAGAGATTGTGTTGAAAGAAATG |
| | | GTTTGCATTCTGTTGTTGATGATGATTTGGATACTGAA |
| | | CATAGAGCTGCTTCTGCTAGAGATTATAAAGATGATGA |
| | | TGATAAATAA |
| 406 | ScMNN2(M1- | ATGTTGCTAACTAAACGTTTCTCTAAGTTGTTCAAGTT |
| | S36)_Fam20c(D9 | GACGTTCATTGTTTTGATACTCTGTGGTTTGTTTGTTAT |
| | 3-R584)_FLAG | CACTAATAAATACATGGATGAAAATACTTCTGATTTTTC |
| | | TTCTGATCCATCTTCTAATTTATCTAGTCACTCTTTGGA |
| | | AAAATTGCCACCTGCTGCTGAGCCTGCTGAAAGAGCTT |
| | | TGAGAGGTAGAGACCCTGGTGCGCTCAGACCACATGA |
| | | TCCTGCTCATAGACCATTGTTGAGAGACCCTGGTCCTA |
| | | GAAGATCTGAATCTCCACCTGGTCCTGGTGGTGATGCT |
| | | TCTTTGTTGGCTAGATTGTTTGAACATCCATTGTATAG |
| | | AGTTGCTGTTCCACCATTGACTGAAGAAGATGTTTTGT |
| | | TTAATGTTAATTCTGATACTAGATTGTCTCCAAAAGCT |
| | | GCTGAAAATCCTGATTGGCCACATGCTGGTGCTGAAGG |
| | | TGCTGAATTTTTGTCTCCTGGTGAAGCGGCTGTTGATT |
| | | CCTATCCAAATTGGTTGAAATTCCACATCGGTATTAAC |
| | | CGATACGAATTGTATTCTAGACATAATCCTGCTATTGA |
| | | AGCTTTGTTGCATGATTTGTCTAGTCAGAGAATTACTT |
| | | CTGTTGCTATGAAGTCTGGTGGTACTCAGTTGAAGTTG |
| | | ATAATGACTTTTCAAAACTATGGGCAAGCTTTGTTTAA |
| | | ACCAATGAAACAAACGAGAGAACAAGAAACTCCACCTG |
| | | ATTTTTTTTATTTTTCGGATTATGAAAGACATAATGCTG |
| | | AAATTGCTGCTTTCCACTTGGACAGAATATTGGATTTT |
| | | CGCAGAGTTCCACCTGTTGCTGGTCGGATGGTTAACAT |
| | | GACTAAGGAAATTAGAGATGTTACTAGAGATAAAAAAT |
| | | TGTGGAGAACTTTTTTCATTTCTCCGGCTAATAATATTT |
| | | GTTTTTACGGGGAATGTTCTTATTATTGTTCTACTGAAC |
| | | ATGCTTTGTGTGGTAAACCTGATCAAATTGAAGGTTCT |
| | | TTGGCTGCTTTTTTGCCTGATTTGTCTTTGGCTAAAAG |
| | | AAAAACTTGGAGAAATCCATGGAGAAGATCTTATCATA |
| | | AAAGAAAAAAAGCTGAATGGGAAGTTGATCCTGATTAT |
| | | TGTGAAGAAGTTAAACAAACTCCACCATATGATTCGTC |
| | | TCATAGAATATTGGACGTCATGGATATGACGATCTTTG |
| | | ACTTTCTGATGGGGAACATGGACAGACATCACTATGAA |
| | | ACATTCGAAAAATTCGGTAATGAAACTTTTATCATCCA |
| | | TTTGGATAATGGTAGAGGTTTTGGTAAATATTCTCATG |
| | | ATGAATTGTCTATTTTGGTTCCATTGCAACAGTGTTGT |
| | | AGAATAAGGAAAAGCACTTACTTAAGATTACAACTCTT |
| | | GGCTAAAGAAGAATATAAATTGTCTTTGTTGATGGCTG |
| | | AATCTTTGAGAGGTGATCAAGTTGCTCCTGTTTTGTAT |
| | | CAACCACATTTGGAAGCTTTGGATAGAAGATTGAGAGT |
| | | TGTTTTGAAAGCTGTTAGAGATTGTGTTGAAAGAAATG |
| | | GTTTGCATTCTGTTGTTGATGATGATTTGGATACTGAA |
| | | CATAGAGCTGCTTCTGCTAGAGATTATAAAGATGATGA |
| | | TGATAAATAA |
| 407 | ScMNN2(M1- | ATGTTGCTAACTAAACGTTTCTCTAAGTTGTTCAAGTT |
| | P97)_Fam20c(D9 | GACGTTCATTGTTTTGATACTCTGTGGTTTGTTTGTTAT |
| | 3-R584)_FLAG | CACTAATAAATACATGGATGAAAATACTTCTGTAAAGG |
| | | AGTATAAAGAGTACCTAGACAGATATGTACAGTCTTAT |
| | | TCCAATAAATATTCTTCTTCTTCTGATGCTGCTTCTGCT |
| | | GATGATTCTACTCCCTTGCGAGACAATGATGAAGCCGG |
| | | TAACGAAAAACTCAAATCCTTTTACAATAATGTTTTTAA |
| | | CTTTTTGATGGTTGATTCACCAGATTTTTCTTCTGATCC |
| | | ATCTTCTAATTTATCTAGTCACTCTTTGGAAAAATTGCC |
| | | ACCTGCTGCTGAGCCTGCTGAAAGAGCTTTGAGAGGTA |
| | | GAGACCCTGGTGCGCTCAGACCACATGATCCTGCTCAT |
| | | AGACCATTGTTGAGAGACCCTGGTCCTAGAAGATCTGA |
| | | ATCTCCACCTGGTCCTGGTGGTGATGCTTCTTTGTTGG |
| | | CTAGATTGTTTGAACATCCATTGTATAGAGTTGCTGTT |
| | | CCACCATTGACTGAAGAAGATGTTTTGTTTAATGTTAA |
| | | TTCTGATACTAGATTGTCTCCAAAAGCTGCTGAAAATC |
| | | CTGATTGGCCACATGCTGGTGCTGAAGGTGCTGAATTT |
| | | TTGTCTCCTGGTGAAGCGGCTGTTGATTCCTATCCAAA |
| | | TTGGTTGAAATTCCACATCGGTATTAACCGATACGAAT |
| | | TGTATTCTAGACATAATCCTGCTATTGAAGCTTTGTTG |
| | | CATGATTTGTCTAGTCAGAGAATTACTTCTGTTGCTAT |
| | | GAAGTCTGGTGGTACTCAGTTGAAGTTGATAATGACTT |
| | | TTCAAAACTATGGGCAAGCTTTGTTTAAACCAATGAAA |
| | | CAAACGAGAGAACAAGAAACTCCACCTGATTTTTTTTA |
| | | TTTTTCGGATTATGAAAGACATAATGCTGAAATTGCTG |
| | | CTTTCCACTTGGACAGAATATTGGATTTTCGCAGAGTT |
| | | CCACCTGTTGCTGGTCGGATGGTTAACATGACTAAGGA |
| | | AATTAGAGATGTTACTAGAGATAAAAAATTGTGGAGAA |
| | | CTTTTTTCATTTCTCCGGCTAATAATATTTGTTTTTACG |
| | | GGGAATGTTCTTATTATTGTTCTACTGAACATGCTTTGT |
| | | GTGGTAAACCTGATCAAATTGAAGGTTCTTTGGCTGCT |
| | | TTTTTGCCTGATTTGTCTTTGGCTAAAAGAAAAACTTG |
| | | GAGAAATCCATGGAGAAGATCTTATCATAAAAGAAAAA |
| | | AAGCTGAATGGGAAGTTGATCCTGATTATTGTGAAGAA |
| | | GTTAAACAAACTCCACCATATGATTCGTCTCATAGAAT |
| | | ATTGGACGTCATGGATATGACGATCTTTGACTTTCTGA |
| | | TGGGGAACATGGACAGACATCACTATGAAACATTCGAA |
| | | AAATTCGGTAATGAAACTTTTATCATCCATTTGGATAAT |
| | | GGTAGAGGTTTTGGTAAATATTCTCATGATGAATTGTC |
| | | TATTTTGGTTCCATTGCAACAGTGTTGTAGAATAAGGA |
| | | AAAGCACTTACTTAAGATTACAACTCTTGGCTAAAGAA |
| | | GAATATAAATTGTCTTTGTTGATGGCTGAATCTTTGAG |
| | | AGGTGATCAAGTTGCTCCTGTTTTGTATCAACCACATT |
| | | TGGAAGCTTTGGATAGAAGATTGAGAGTTGTTTTGAAA |
| | | GCTGTTAGAGATTGTGTTGAAAGAAATGGTTTGCATTC |
| | | TGTTGTTGATGATGATTTGGATACTGAACATAGAGCTG |
| | | CTTCTGCTAGAGATTATAAAGATGATGATGATAAATAA |
| 408 | ScMNN2(M1- | ATGTTGCTAACTAAACGTTTCTCTAAGTTGTTCAAGTT |
| | S150)_Fam20c(D | GACGTTCATTGTTTTGATACTCTGTGGTTTGTTTGTTAT |
| | 93-R584)_FLAG | CACTAATAAATACATGGATGAAAATACTTCTGTAAAGG |
| | | AGTATAAAGAGTACCTAGACAGATATGTACAGTCTTAT |
| | | TCCAATAAATATTCTTCTTCTTCTGATGCTGCTTCTGCT |
| | | GATGATTCTACTCCCTTGCGAGACAATGATGAAGCCGG |
| | | TAACGAAAAACTCAAATCCTTTTACAATAATGTTTTTAA |
| | | CTTTTTGATGGTTGATTCACCAAAAGGTTCTACTGCTA |
| | | AACAATATAATGAAGCTTGTTTGTTGAAAGGTGATATT |
| | | GGAGATAGACCTGATCATTATAAGGATTTGTACAAATT |
| | | GTCTGCTAAAGAATTGTCTAAATGTTTGGAATTGTCTC |
| | | CTGATGAAGTTGCTTCTTTGACTAAATCTGATTTTTCTT |
| | | CTGATCCATCTTCTAATTTATCTAGTCACTCTTTGGAAA |
| | | AATTGCCACCTGCTGCTGAGCCTGCTGAAAGAGCTTTG |
| | | AGAGGTAGAGACCCTGGTGCGCTCAGACCACATGATC |
| | | CTGCTCATAGACCATTGTTGAGAGACCCTGGTCCTAGA |
| | | AGATCTGAATCTCCACCTGGTCCTGGTGGTGATGCTTC |
| | | TTTGTTGGCTAGATTGTTTGAACATCCATTGTATAGAG |
| | | TTGCTGTTCCACCATTGACTGAAGAAGATGTTTTGTTT |
| | | AATGTTAATTCTGATACTAGATTGTCTCCAAAAGCTGC |
| | | TGAAAATCCTGATTGGCCACATGCTGGTGCTGAAGGTG |
| | | CTGAATTTTTGTCTCCTGGTGAAGCGGCTGTTGATTCC |
| | | TATCCAAATTGGTTGAAATTCCACATCGGTATTAACCG |
| | | ATACGAATTGTATTCTAGACATAATCCTGCTATTGAAG |
| | | CTTTGTTGCATGATTTGTCTAGTCAGAGAATTACTTCT |
| | | GTTGCTATGAAGTCTGGTGGTACTCAGTTGAAGTTGAT |
| | | AATGACTTTTCAAAACTATGGGCAAGCTTTGTTTAAAC |
| | | CAATGAAACAAACGAGAGAACAAGAAACTCCACCTGAT |
| | | TTTTTTTATTTTTCGGATTATGAAAGACATAATGCTGAA |
| | | ATTGCTGCTTTCCACTTGGACAGAATATTGGATTTTCG |
| | | CAGAGTTCCACCTGTTGCTGGTCGGATGGTTAACATGA |
| | | CTAAGGAAATTAGAGATGTTACTAGAGATAAAAAATTG |
| | | TGGAGAACTTTTTTCATTTCTCCGGCTAATAATATTTGT |
| | | TTTTACGGGGAATGTTCTTATTATTGTTCTACTGAACAT |
| | | GCTTTGTGTGGTAAACCTGATCAAATTGAAGGTTCTTT |
| | | GGCTGCTTTTTTGCCTGATTTGTCTTTGGCTAAAAGAA |
| | | AAACTTGGAGAAATCCATGGAGAAGATCTTATCATAAA |
| | | AGAAAAAAAGCTGAATGGGAAGTTGATCCTGATTATTG |
| | | TGAAGAAGTTAAACAAACTCCACCATATGATTCGTCTC |
| | | ATAGAATATTGGACGTCATGGATATGACGATCTTTGAC |
| | | TTTCTGATGGGGAACATGGACAGACATCACTATGAAAC |
| | | ATTCGAAAAATTCGGTAATGAAACTTTTATCATCCATTT |
| | | GGATAATGGTAGAGGTTTTGGTAAATATTCTCATGATG |
| | | AATTGTCTATTTTGGTTCCATTGCAACAGTGTTGTAGA |
| | | ATAAGGAAAAGCACTTACTTAAGATTACAACTCTTGGC |
| | | TAAAGAAGAATATAAATTGTCTTTGTTGATGGCTGAAT |
| | | CTTTGAGAGGTGATCAAGTTGCTCCTGTTTTGTATCAA |
| | | CCACATTTGGAAGCTTTGGATAGAAGATTGAGAGTTGT |
| | | TTTGAAAGCTGTTAGAGATTGTGTTGAAAGAAATGGTT |
| | | TGCATTCTGTTGTTGATGATGATTTGGATACTGAACAT |
| | | AGAGCTGCTTCTGCTAGAGATTATAAAGATGATGATGA |
| | | TAAATAA |
| 409 | ScMNN1(M1- | ATGTTGGCTTTGAGAAGATTTATCTTGAATCAGAGATC |
| | A42)_Fam20c(D | TTTGAGATCTTGTACTATACCAATATTGGTTGGGGCTT |
| | 93-R584)_FLAG | TGATTATTATTTTGGTTTTGTTTCAATTGGTCACTCATA |
| | | GAAACGATGCTGATTTTTCTTCTGATCCATCTTCTAATT |
| | | TATCTAGTCACTCTTTGGAAAAATTGCCACCTGCTGCT |
| | | GAGCCTGCTGAAAGAGCTTTGAGAGGTAGAGACCCTG |
| | | GTGCGCTCAGACCACATGATCCTGCTCATAGACCATTG |
| | | TTGAGAGACCCTGGTCCTAGAAGATCTGAATCTCCACC |
| | | TGGTCCTGGTGGTGATGCTTCTTTGTTGGCTAGATTGT |
| | | TTGAACATCCATTGTATAGAGTTGCTGTTCCACCATTG |
| | | ACTGAAGAAGATGTTTTGTTTAATGTTAATTCTGATACT |
| | | AGATTGTCTCCAAAAGCTGCTGAAAATCCTGATTGGCC |
| | | ACATGCTGGTGCTGAAGGTGCTGAATTTTTGTCTCCTG |
| | | GTGAAGCGGCTGTTGATTCCTATCCAAATTGGTTGAAA |
| | | TTCCACATCGGTATTAACCGATACGAATTGTATTCTAG |
| | | ACATAATCCTGCTATTGAAGCTTTGTTGCATGATTTGT |
| | | CTAGTCAGAGAATTACTTCTGTTGCTATGAAGTCTGGT |
| | | GGTACTCAGTTGAAGTTGATAATGACTTTTCAAAACTA |
| | | TGGGCAAGCTTTGTTTAAACCAATGAAACAAACGAGAG |
| | | AACAAGAAACTCCACCTGATTTTTTTTATTTTTCGGATT |
| | | ATGAAAGACATAATGCTGAAATTGCTGCTTTCCACTTG |
| | | GACAGAATATTGGATTTTCGCAGAGTTCCACCTGTTGC |
| | | TGGTCGGATGGTTAACATGACTAAGGAAATTAGAGATG |
| | | TTACTAGAGATAAAAAATTGTGGAGAACTTTTTTCATTT |
| | | CTCCGGCTAATAATATTTGTTTTTACGGGGAATGTTCT |
| | | TATTATTGTTCTACTGAACATGCTTTGTGTGGTAAACCT |
| | | GATCAAATTGAAGGTTCTTTGGCTGCTTTTTTGCCTGA |
| | | TTTGTCTTTGGCTAAAAGAAAAACTTGGAGAAATCCAT |
| | | GGAGAAGATCTTATCATAAAAGAAAAAAAGCTGAATGG |
| | | GAAGTTGATCCTGATTATTGTGAAGAAGTTAAACAAAC |
| | | TCCACCATATGATTCGTCTCATAGAATATTGGACGTCA |
| | | TGGATATGACGATCTTTGACTTTCTGATGGGGAACATG |
| | | GACAGACATCACTATGAAACATTCGAAAAATTCGGTAA |
| | | TGAAACTTTTATCATCCATTTGGATAATGGTAGAGGTT |
| | | TTGGTAAATATTCTCATGATGAATTGTCTATTTTGGTTC |
| | | CATTGCAACAGTGTTGTAGAATAAGGAAAAGCACTTAC |
| | | TTAAGATTACAACTCTTGGCTAAAGAAGAATATAAATT |
| | | GTCTTTGTTGATGGCTGAATCTTTGAGAGGTGATCAAG |
| | | TTGCTCCTGTTTTGTATCAACCACATTTGGAAGCTTTG |
| | | GATAGAAGATTGAGAGTTGTTTTGAAAGCTGTTAGAGA |
| | | TTGTGTTGAAAGAAATGGTTTGCATTCTGTTGTTGATG |
| | | ATGATTTGGATACTGAACATAGAGCTGCTTCTGCTAGA |
| | | GATTATAAAGATGATGATGATAAATAA |
| 410 | ScMNN1(M1- | ATGTTGGCTTTGAGAAGATTTATCTTGAATCAGAGATC |
| | Q93)_Fam20c(D | TTTGAGATCTTGTACTATACCAATATTGGTTGGGGCTT |
| | 93-R584)_FLAG | TGATTATTATTTTGGTTTTGTTTCAATTGGTCACTCATA |
| | | GAAACGATGCTCTCATTAGATCCTCTAATGTTAATTCT |
| | | ACCAACAAAAAAACACTCAAGGATGCTGATCCAAAAGT |
| | | TTTGATTGAAGCTTTTGGTTCTCCTGAAGTTGATCCTG |
| | | TTGATACTATTCCTGTTTCTCCACTTGAATTGGTGCCAT |
| | | TTTACGATCAAGATTTTTCTTCTGATCCATCTTCTAATT |
| | | TATCTAGTCACTCTTTGGAAAAATTGCCACCTGCTGCT |
| | | GAGCCTGCTGAAAGAGCTTTGAGAGGTAGAGACCCTG |
| | | GTGCGCTCAGACCACATGATCCTGCTCATAGACCATTG |
| | | TTGAGAGACCCTGGTCCTAGAAGATCTGAATCTCCACC |
| | | TGGTCCTGGTGGTGATGCTTCTTTGTTGGCTAGATTGT |
| | | TTGAACATCCATTGTATAGAGTTGCTGTTCCACCATTG |
| | | ACTGAAGAAGATGTTTTGTTTAATGTTAATTCTGATACT |
| | | AGATTGTCTCCAAAAGCTGCTGAAAATCCTGATTGGCC |
| | | ACATGCTGGTGCTGAAGGTGCTGAATTTTTGTCTCCTG |
| | | GTGAAGCGGCTGTTGATTCCTATCCAAATTGGTTGAAA |
| | | TTCCACATCGGTATTAACCGATACGAATTGTATTCTAG |
| | | ACATAATCCTGCTATTGAAGCTTTGTTGCATGATTTGT |
| | | CTAGTCAGAGAATTACTTCTGTTGCTATGAAGTCTGGT |
| | | GGTACTCAGTTGAAGTTGATAATGACTTTTCAAAACTA |
| | | TGGGCAAGCTTTGTTTAAACCAATGAAACAAACGAGAG |
| | | AACAAGAAACTCCACCTGATTTTTTTTATTTTTCGGATT |
| | | ATGAAAGACATAATGCTGAAATTGCTGCTTTCCACTTG |
| | | GACAGAATATTGGATTTTCGCAGAGTTCCACCTGTTGC |
| | | TGGTCGGATGGTTAACATGACTAAGGAAATTAGAGATG |
| | | TTACTAGAGATAAAAAATTGTGGAGAACTTTTTTCATTT |
| | | CTCCGGCTAATAATATTTGTTTTTACGGGGAATGTTCT |
| | | TATTATTGTTCTACTGAACATGCTTTGTGTGGTAAACCT |
| | | GATCAAATTGAAGGTTCTTTGGCTGCTTTTTTGCCTGA |
| | | TTTGTCTTTGGCTAAAAGAAAAACTTGGAGAAATCCAT |
| | | GGAGAAGATCTTATCATAAAAGAAAAAAAGCTGAATGG |
| | | GAAGTTGATCCTGATTATTGTGAAGAAGTTAAACAAAC |
| | | TCCACCATATGATTCGTCTCATAGAATATTGGACGTCA |
| | | TGGATATGACGATCTTTGACTTTCTGATGGGGAACATG |
| | | GACAGACATCACTATGAAACATTCGAAAAATTCGGTAA |
| | | TGAAACTTTTATCATCCATTTGGATAATGGTAGAGGTT |
| | | TTGGTAAATATTCTCATGATGAATTGTCTATTTTGGTTC |
| | | CATTGCAACAGTGTTGTAGAATAAGGAAAAGCACTTAC |
| | | TTAAGATTACAACTCTTGGCTAAAGAAGAATATAAATT |
| | | GTCTTTGTTGATGGCTGAATCTTTGAGAGGTGATCAAG |
| | | TTGCTCCTGTTTTGTATCAACCACATTTGGAAGCTTTG |
| | | GATAGAAGATTGAGAGTTGTTTTGAAAGCTGTTAGAGA |
| | | TTGTGTTGAAAGAAATGGTTTGCATTCTGTTGTTGATG |
| | | ATGATTTGGATACTGAACATAGAGCTGCTTCTGCTAGA |
| | | GATTATAAAGATGATGATGATAAATAA |
| 411 | ScMNN1(M1- | ATGTTGGCTTTGAGAAGATTTATCTTGAATCAGAGATC |
| | G153)_Fam20c( | TTTGAGATCTTGTACTATACCAATATTGGTTGGGGCTT |
| | D93- | TGATTATTATTTTGGTTTTGTTTCAATTGGTCACTCATA |
| | R584)_FLAG | GAAACGATGCTCTCATTAGATCCTCTAATGTTAATTCT |
| | | ACCAACAAAAAAACACTCAAGGATGCTGATCCAAAAGT |
| | | TTTGATTGAAGCTTTTGGTTCTCCTGAAGTTGATCCTG |
| | | TTGATACTATTCCTGTTTCTCCACTTGAATTGGTGCCAT |
| | | TTTACGATCAATCTATTGATACTAAGAGGTCCTCTTCAT |
| | | GGTTGATAAACAAAAAGGGTTATTATAAACACTTCAAC |
| | | GAACTGTCTTTGACGGACAGATGCAAGTTCTATTTTAG |
| | | AACATTGTATACTCTAGACGATGAGTGGACTAACTCTG |
| | | TTAAAAAATTGGAATATTCAATTAATGATAATGAAGGT |
| | | GATTTTTCTTCTGATCCATCTTCTAATTTATCTAGTCAC |
| | | TCTTTGGAAAAATTGCCACCTGCTGCTGAGCCTGCTGA |
| | | AAGAGCTTTGAGAGGTAGAGACCCTGGTGCGCTCAGA |
| | | CCACATGATCCTGCTCATAGACCATTGTTGAGAGACCC |
| | | TGGTCCTAGAAGATCTGAATCTCCACCTGGTCCTGGTG |
| | | GTGATGCTTCTTTGTTGGCTAGATTGTTTGAACATCCA |
| | | TTGTATAGAGTTGCTGTTCCACCATTGACTGAAGAAGA |
| | | TGTTTTGTTTAATGTTAATTCTGATACTAGATTGTCTCC |
| | | AAAAGCTGCTGAAAATCCTGATTGGCCACATGCTGGTG |
| | | CTGAAGGTGCTGAATTTTTGTCTCCTGGTGAAGCGGCT |
| | | GTTGATTCCTATCCAAATTGGTTGAAATTCCACATCGG |
| | | TATTAACCGATACGAATTGTATTCTAGACATAATCCTG |
| | | CTATTGAAGCTTTGTTGCATGATTTGTCTAGTCAGAGA |
| | | ATTACTTCTGTTGCTATGAAGTCTGGTGGTACTCAGTT |
| | | GAAGTTGATAATGACTTTTCAAAACTATGGGCAAGCTT |
| | | TGTTTAAACCAATGAAACAAACGAGAGAACAAGAAACT |
| | | CCACCTGATTTTTTTTATTTTTCGGATTATGAAAGACAT |
| | | AATGCTGAAATTGCTGCTTTCCACTTGGACAGAATATT |
| | | GGATTTTCGCAGAGTTCCACCTGTTGCTGGTCGGATGG |
| | | TTAACATGACTAAGGAAATTAGAGATGTTACTAGAGAT |
| | | AAAAAATTGTGGAGAACTTTTTTCATTTCTCCGGCTAA |
| | | TAATATTTGTTTTTACGGGGAATGTTCTTATTATTGTTC |
| | | TACTGAACATGCTTTGTGTGGTAAACCTGATCAAATTG |
| | | AAGGTTCTTTGGCTGCTTTTTTGCCTGATTTGTCTTTGG |
| | | CTAAAAGAAAAACTTGGAGAAATCCATGGAGAAGATCT |
| | | TATCATAAAAGAAAAAAAGCTGAATGGGAAGTTGATCC |
| | | TGATTATTGTGAAGAAGTTAAACAAACTCCACCATATG |
| | | ATTCGTCTCATAGAATATTGGACGTCATGGATATGACG |
| | | ATCTTTGACTTTCTGATGGGGAACATGGACAGACATCA |
| | | CTATGAAACATTCGAAAAATTCGGTAATGAAACTTTTA |
| | | TCATCCATTTGGATAATGGTAGAGGTTTTGGTAAATAT |
| | | TCTCATGATGAATTGTCTATTTTGGTTCCATTGCAACA |
| | | GTGTTGTAGAATAAGGAAAAGCACTTACTTAAGATTAC |
| | | AACTCTTGGCTAAAGAAGAATATAAATTGTCTTTGTTG |
| | | ATGGCTGAATCTTTGAGAGGTGATCAAGTTGCTCCTGT |
| | | TTTGTATCAACCACATTTGGAAGCTTTGGATAGAAGAT |
| | | TGAGAGTTGTTTTGAAAGCTGTTAGAGATTGTGTTGAA |
| | | AGAAATGGTTTGCATTCTGTTGTTGATGATGATTTGGA |
| | | TACTGAACATAGAGCTGCTTCTGCTAGAGATTATAAAG |
| | | ATGATGATGATAAATAA |
| 412 | ScMNN6(M1- | ATGCATGTTTTGTTGTCTAAAAAGATTGCTAGATTTTTG |
| | P30)_Fam20c(D9 | TTGATATCTTTTGTTTTTGTTTTGGCTTTGATGGTTACC |
| | 3-R584)_FLAG | ATTAACCATCCGGATTTTTCTTCTGATCCATCTTCTAAT |
| | | TTATCTAGTCACTCTTTGGAAAAATTGCCACCTGCTGC |
| | | TGAGCCTGCTGAAAGAGCTTTGAGAGGTAGAGACCCT |
| | | GGTGCGCTCAGACCACATGATCCTGCTCATAGACCATT |
| | | GTTGAGAGACCCTGGTCCTAGAAGATCTGAATCTCCAC |
| | | CTGGTCCTGGTGGTGATGCTTCTTTGTTGGCTAGATTG |
| | | TTTGAACATCCATTGTATAGAGTTGCTGTTCCACCATT |
| | | GACTGAAGAAGATGTTTTGTTTAATGTTAATTCTGATA |
| | | CTAGATTGTCTCCAAAAGCTGCTGAAAATCCTGATTGG |
| | | CCACATGCTGGTGCTGAAGGTGCTGAATTTTTGTCTCC |
| | | TGGTGAAGCGGCTGTTGATTCCTATCCAAATTGGTTGA |
| | | AATTCCACATCGGTATTAACCGATACGAATTGTATTCT |
| | | AGACATAATCCTGCTATTGAAGCTTTGTTGCATGATTT |
| | | GTCTAGTCAGAGAATTACTTCTGTTGCTATGAAGTCTG |
| | | GTGGTACTCAGTTGAAGTTGATAATGACTTTTCAAAAC |
| | | TATGGGCAAGCTTTGTTTAAACCAATGAAACAAACGAG |
| | | AGAACAAGAAACTCCACCTGATTTTTTTTATTTTTCGGA |
| | | TTATGAAAGACATAATGCTGAAATTGCTGCTTTCCACT |
| | | TGGACAGAATATTGGATTTTCGCAGAGTTCCACCTGTT |
| | | GCTGGTCGGATGGTTAACATGACTAAGGAAATTAGAGA |
| | | TGTTACTAGAGATAAAAAATTGTGGAGAACTTTTTTCA |
| | | TTTCTCCGGCTAATAATATTTGTTTTTACGGGGAATGTT |
| | | CTTATTATTGTTCTACTGAACATGCTTTGTGTGGTAAAC |
| | | CTGATCAAATTGAAGGTTCTTTGGCTGCTTTTTTGCCT |
| | | GATTTGTCTTTGGCTAAAAGAAAAACTTGGAGAAATCC |
| | | ATGGAGAAGATCTTATCATAAAAGAAAAAAAGCTGAAT |
| | | GGGAAGTTGATCCTGATTATTGTGAAGAAGTTAAACAA |
| | | ACTCCACCATATGATTCGTCTCATAGAATATTGGACGT |
| | | CATGGATATGACGATCTTTGACTTTCTGATGGGGAACA |
| | | TGGACAGACATCACTATGAAACATTCGAAAAATTCGGT |
| | | AATGAAACTTTTATCATCCATTTGGATAATGGTAGAGG |
| | | TTTTGGTAAATATTCTCATGATGAATTGTCTATTTTGGT |
| | | TCCATTGCAACAGTGTTGTAGAATAAGGAAAAGCACTT |
| | | ACTTAAGATTACAACTCTTGGCTAAAGAAGAATATAAA |
| | | TTGTCTTTGTTGATGGCTGAATCTTTGAGAGGTGATCA |
| | | AGTTGCTCCTGTTTTGTATCAACCACATTTGGAAGCTT |
| | | TGGATAGAAGATTGAGAGTTGTTTTGAAAGCTGTTAGA |
| | | GATTGTGTTGAAAGAAATGGTTTGCATTCTGTTGTTGA |
| | | TGATGATTTGGATACTGAACATAGAGCTGCTTCTGCTA |
| | | GAGATTATAAAGATGATGATGATAAATAA |
| 413 | ScMNN6(M1- | ATGCATGTTTTGTTGTCTAAAAAGATTGCTAGATTTTTG |
| | V85)_Fam20c(D | TTGATATCTTTTGTTTTTGTTTTGGCTTTGATGGTTACC |
| | 93-R584)_FLAG | ATTAACCATCCGAAGACTAAACAAATGTCTGAACAATA |
| | | TGTTACTCCATATTTGCCAAAATCTTTGCAACCAATTGC |
| | | TAAAATTTCTGCTGAAGAACAAAGAAGAATTCAATCTG |
| | | AACAAGAAGAAGCTGAATTGAAACAATCTTTGGAAGGT |
| | | GAAGCAATAAGAAATGCTACCGTTGATTTTTCTTCTGA |
| | | TCCATCTTCTAATTTATCTAGTCACTCTTTGGAAAAATT |
| | | GCCACCTGCTGCTGAGCCTGCTGAAAGAGCTTTGAGA |
| | | GGTAGAGACCCTGGTGCGCTCAGACCACATGATCCTG |
| | | CTCATAGACCATTGTTGAGAGACCCTGGTCCTAGAAGA |
| | | TCTGAATCTCCACCTGGTCCTGGTGGTGATGCTTCTTT |
| | | GTTGGCTAGATTGTTTGAACATCCATTGTATAGAGTTG |
| | | CTGTTCCACCATTGACTGAAGAAGATGTTTTGTTTAAT |
| | | GTTAATTCTGATACTAGATTGTCTCCAAAAGCTGCTGA |
| | | AAATCCTGATTGGCCACATGCTGGTGCTGAAGGTGCTG |
| | | AATTTTTGTCTCCTGGTGAAGCGGCTGTTGATTCCTAT |
| | | CCAAATTGGTTGAAATTCCACATCGGTATTAACCGATA |
| | | CGAATTGTATTCTAGACATAATCCTGCTATTGAAGCTT |
| | | TGTTGCATGATTTGTCTAGTCAGAGAATTACTTCTGTT |
| | | GCTATGAAGTCTGGTGGTACTCAGTTGAAGTTGATAAT |
| | | GACTTTTCAAAACTATGGGCAAGCTTTGTTTAAACCAA |
| | | TGAAACAAACGAGAGAACAAGAAACTCCACCTGATTTT |
| | | TTTTATTTTTCGGATTATGAAAGACATAATGCTGAAATT |
| | | GCTGCTTTCCACTTGGACAGAATATTGGATTTTCGCAG |
| | | AGTTCCACCTGTTGCTGGTCGGATGGTTAACATGACTA |
| | | AGGAAATTAGAGATGTTACTAGAGATAAAAAATTGTGG |
| | | AGAACTTTTTTCATTTCTCCGGCTAATAATATTTGTTTT |
| | | TACGGGGAATGTTCTTATTATTGTTCTACTGAACATGC |
| | | TTTGTGTGGTAAACCTGATCAAATTGAAGGTTCTTTGG |
| | | CTGCTTTTTTGCCTGATTTGTCTTTGGCTAAAAGAAAA |
| | | ACTTGGAGAAATCCATGGAGAAGATCTTATCATAAAAG |
| | | AAAAAAAGCTGAATGGGAAGTTGATCCTGATTATTGTG |
| | | AAGAAGTTAAACAAACTCCACCATATGATTCGTCTCAT |
| | | AGAATATTGGACGTCATGGATATGACGATCTTTGACTT |
| | | TCTGATGGGGAACATGGACAGACATCACTATGAAACAT |
| | | TCGAAAAATTCGGTAATGAAACTTTTATCATCCATTTG |
| | | GATAATGGTAGAGGTTTTGGTAAATATTCTCATGATGA |
| | | ATTGTCTATTTTGGTTCCATTGCAACAGTGTTGTAGAA |
| | | TAAGGAAAAGCACTTACTTAAGATTACAACTCTTGGCT |
| | | AAAGAAGAATATAAATTGTCTTTGTTGATGGCTGAATC |
| | | TTTGAGAGGTGATCAAGTTGCTCCTGTTTTGTATCAAC |
| | | CACATTTGGAAGCTTTGGATAGAAGATTGAGAGTTGTT |
| | | TTGAAAGCTGTTAGAGATTGTGTTGAAAGAAATGGTTT |
| | | GCATTCTGTTGTTGATGATGATTTGGATACTGAACATA |
| | | GAGCTGCTTCTGCTAGAGATTATAAAGATGATGATGAT |
| | | AAATAA |
| 414 | ScMNN6(M1- | ATGCATGTTTTGTTGTCTAAAAAGATTGCTAGATTTTTG |
| | E160)_Fam20c(D | TTGATATCTTTTGTTTTTGTTTTGGCTTTGATGGTTACC |
| | 93-R584)_FLAG | ATTAACCATCCGAAGACTAAACAAATGTCTGAACAATA |
| | | TGTTACTCCATATTTGCCAAAATCTTTGCAACCAATTGC |
| | | TAAAATTTCTGCTGAAGAACAAAGAAGAATTCAATCTG |
| | | AACAAGAAGAAGCTGAATTGAAACAATCTTTGGAAGGT |
| | | GAAGCAATAAGAAATGCTACCGTTAACGCCATTAAAGA |
| | | AAAAATTAAATCTTATGGTGGTAATGAAACTACTTTGG |
| | | GTTTTATGGTTCCATCTTATATTAATCATAGAGGTTCTC |
| | | CACCCAAAGCTTGCTTCGTTTCATTGATCACAGAAAGG |
| | | GACTCTATGACTCAAATCTTGCAATCTATAGATGAGGT |
| | | CCAAGTCAAGTTTAACAAAAATTTTGCTTATCCATGGG |
| | | TTTTTATTTCTCAAGGTGAAGATTTTTCTTCTGATCCAT |
| | | CTTCTAATTTATCTAGTCACTCTTTGGAAAAATTGCCAC |
| | | CTGCTGCTGAGCCTGCTGAAAGAGCTTTGAGAGGTAG |
| | | AGACCCTGGTGCGCTCAGACCACATGATCCTGCTCATA |
| | | GACCATTGTTGAGAGACCCTGGTCCTAGAAGATCTGAA |
| | | TCTCCACCTGGTCCTGGTGGTGATGCTTCTTTGTTGGC |
| | | TAGATTGTTTGAACATCCATTGTATAGAGTTGCTGTTC |
| | | CACCATTGACTGAAGAAGATGTTTTGTTTAATGTTAAT |
| | | TCTGATACTAGATTGTCTCCAAAAGCTGCTGAAAATCC |
| | | TGATTGGCCACATGCTGGTGCTGAAGGTGCTGAATTTT |
| | | TGTCTCCTGGTGAAGCGGCTGTTGATTCCTATCCAAAT |
| | | TGGTTGAAATTCCACATCGGTATTAACCGATACGAATT |
| | | GTATTCTAGACATAATCCTGCTATTGAAGCTTTGTTGC |
| | | ATGATTIGTCTAGTCAGAGAATTACTTCTGTTGCTATG |
| | | AAGTCTGGTGGTACTCAGTTGAAGTTGATAATGACTTT |
| | | TCAAAACTATGGGCAAGCTTTGTTTAAACCAATGAAAC |
| | | AAACGAGAGAACAAGAAACTCCACCTGATTTTTTTTAT |
| | | TTTTCGGATTATGAAAGACATAATGCTGAAATTGCTGC |
| | | TTTCCACTTGGACAGAATATTGGATTTTCGCAGAGTTC |
| | | CACCTGTTGCTGGTCGGATGGTTAACATGACTAAGGAA |
| | | ATTAGAGATGTTACTAGAGATAAAAAATTGTGGAGAAC |
| | | TTTTTTCATTTCTCCGGCTAATAATATTTGTTTTTACGG |
| | | GGAATGTTCTTATTATTGTTCTACTGAACATGCTTTGTG |
| | | TGGTAAACCTGATCAAATTGAAGGTTCTTTGGCTGCTT |
| | | TTTTGCCTGATTTGTCTTTGGCTAAAAGAAAAACTTGG |
| | | AGAAATCCATGGAGAAGATCTTATCATAAAAGAAAAAA |
| | | AGCTGAATGGGAAGTTGATCCTGATTATTGTGAAGAAG |
| | | TTAAACAAACTCCACCATATGATTCGTCTCATAGAATA |
| | | TTGGACGTCATGGATATGACGATCTTTGACTTTCTGAT |
| | | GGGGAACATGGACAGACATCACTATGAAACATTCGAAA |
| | | AATTCGGTAATGAAACTTTTATCATCCATTTGGATAAT |
| | | GGTAGAGGTTTTGGTAAATATTCTCATGATGAATTGTC |
| | | TATTTTGGTTCCATTGCAACAGTGTTGTAGAATAAGGA |
| | | AAAGCACTTACTTAAGATTACAACTCTTGGCTAAAGAA |
| | | GAATATAAATTGTCTTTGTTGATGGCTGAATCTTTGAG |
| | | AGGTGATCAAGTTGCTCCTGTTTTGTATCAACCACATT |
| | | TGGAAGCTTTGGATAGAAGATTGAGAGTTGTTTTGAAA |
| | | GCTGTTAGAGATTGTGTTGAAAGAAATGGTTTGCATTC |
| | | TGTTGTTGATGATGATTTGGATACTGAACATAGAGCTG |
| | | CTTCTGCTAGAGATTATAAAGATGATGATGATAAATAA |

### Example 7: Secretion of Human Serine/Threonine Protein Kinase Fam20c with Pichia pastoris

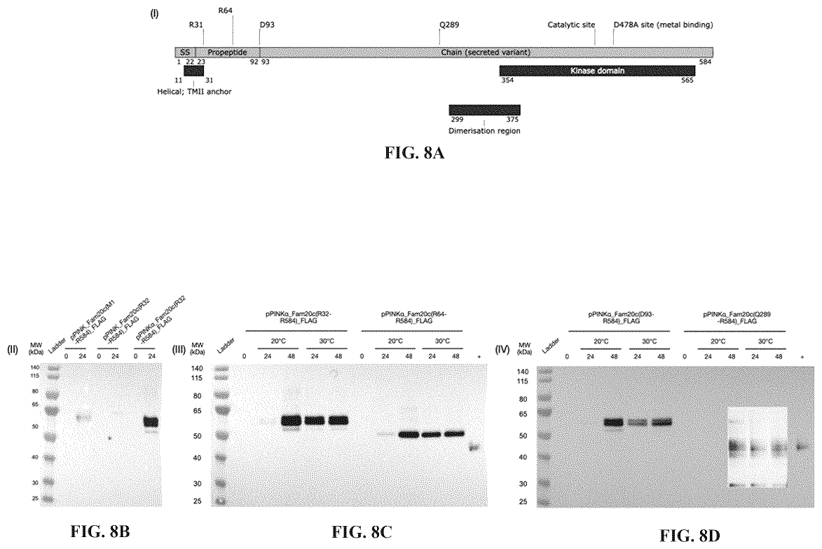

Recombinant vectors encoding human various forms of human serine/threonine protein kinase Fam20c (huFam20c) were generated (FIG. 8I). The DNA sequence for huFam20c was ordered as a DNA fragment (SEQ ID NO: 185) and codon optimized for expression in Pichia pastoris. From this, four fragments were amplified using PCR: (i) Fam20c (R32-R584), (ii) Fam20c (R64-R584), (iii) Fam20c (D93-R584) and (iv) Fam20c (Q289-R584).

Each was cloned into the pPINKα-HC commercial vector, in frame with an N-terminal alpha mating factor (α) secretion signal (SEQ ID NO: 312) and a C-terminal FLAG tag (SEQ ID NO: 10), generating vectors: (i) pPINKα_Fam20c (R32-R584)_FLAG, (ii) pPINKα_Fam20c (R64-R584) FLAG, (iii) pPINKα_Fam20c (D93-R584)_FLAG and (iv) pPINKα_Fam20c (Q289-R584) FLAG. Additionally, M1-R584 and R32-R584 were cloned into the pPINKα expression vector, excluding the N-terminal alpha mating factor secretion signal, but retaining the C-terminal FLAG tag, generating vectors pPINK_Fam20c (M1-R584)_FLAG and pPINK_Fam20c (R32-R584) FLAG. For pPINK_Fam20c (R32-R584) FLAG, an additional N-terminal methionine was included to enable protein translation.

(SEQ ID NO: 312)
MRFPSIFTAVLFAASSALAAPVNTTTEDETAQIPAEAVIGYSDLEGDFD
VAVLPFSNSTNNGLLFINTTIASIAAKEEGVSLEKR.

The vectors were transformed into PichiaPink™ Strain 4 (Δade2, Δprb1, Δpep4), and putative strains confirmed by colony PCR. For each strain, clones were grown overnight in BMGY (non-induction media), diluted to an OD600 of 1.0 in BMMY (induction media) and expressed for 48 hours at 20° C. and 30° C. At 0, 24 and 48 hours, samples of the supernatant were taken. At 24 and 48 hours, cultures were supplemented with additional methanol to maintain induction conditions.

Supernatant samples were TCA precipitated, electrophoresed in denaturing conditions with a NuPAGE 4-12% Bis-Tris protein gel, transferred to a nitrocellulose blot, blocked with 5% milk in TBS-T and probed with HRP-conjugated Anti-DYKDDDDK Mouse Monoclonal Antibody (“DYKDDDDK” is disclosed as SEQ ID NO: 10). Chemiluminescent detection was performed with ECL substrate kit (High Sensitivity). A positive control, of a FLAG-tagged recombinant protein, was included on each blot to confirm functionality of the Western Blot.

Shown in FIG. 8B-8D are Western Blots detecting extracellular expression of various forms of human serine/threonine protein kinase Fam20c after 24 and 48 hours of methanol induction at 20° C. and 30° C. The expression time points and induction temperature are indicated at the top of each lane. FIG. 8 shows secretion of various forms of human serine/threonine protein kinase Fam20c by Pichia pastoris cells.

### Example 8: In Vivo Phosphorylation with Engineered Human Serine/Threonine Protein Kinase Fam20c

Three fragments of human serine/threonine protein kinase Fam20c (huFam20c), codon optimized for expression in Pichia pastoris, were amplified using PCR: (i) R32-R584, (ii) R64-R584 and (iii) D93-R584. These were cloned into the commercially available pd915 vector, deleting the N-terminal alpha mating factor but retaining the C-terminal FLAG tag, generating vectors: (i) pd915_Fam20c (R32-R584) FLAG, (ii) pd915_Fam20c (R64-R584) FLAG and (iii) pd915_Fam20c (D93-R584)_FLAG. For each of these vectors, three truncations of five different fungal localization sequences were cloned at the N-terminus of the Fam20c as described in Example 7. Additionally, variants were generated with an N-terminal methionine to enable translation of the non-localized controls. Finally, the complete M1-R584 sequence was cloned into pd915, deleting the N-terminal alpha mating factor in-frame with a C-terminal MYC tag, generating the vector pd915_Fam20c (M1-R584)_MYC. A list of the engineered huFam20c constructs generated are shown in Table 10. A C-terminal MYC tag can be added to any of the engineered huFam20c constructs in Table 10.

The DNA sequence for human osteopontin (SPP1) was ordered as a DNA fragment, codon optimized for expression in Pichia pastoris. From this, a single truncation (G158-N314) was amplified using PCR. Additionally, the DNA sequence for TRX was ordered as a DNA fragment, codon optimized for expression in Pichia pastoris. These two fragments were cloned into the pPINKα-HC commercial vector (where the AOX1 promoter has been exchanged for the GAP promoter), in frame with an N-terminal alpha mating factor (α) secretion signal, an N-terminal FLAG tag and a C-terminal HiBiT tag, generating the vector pPINKα_FLAG_TRX_SPP1 (G158-N314)_HiBiT.

The pPINKα FLAG_TRX_SPP1 (G158-N314) HiBIT vector was transformed into PichiaPink™ Strain 4 (Δade2, Δprb1, Δpep4), and putative strains confirmed by colony PCR. For each strain, clones were grown overnight in YPD, diluted to an OD600 of 1.0 in fresh YPD and expressed for 48 hours at 30° C. Confirmation of protein expression using electrophoresis and Western blotting was performed as described in Example 7.

A strain with high expression of TRX_SPP1 (G158-N314) HiBIT was selected and competent cells were prepared. The panel of vectors containing engineered Fam20c proteins and corresponding unlocalized controls were transformed into the strain, and putative strains were confirmed by colony PCR. For each strain, clones were grown overnight in YPD, diluted to an OD600 of 1.0 in fresh YPD and expressed for 48 hours at 30° C.

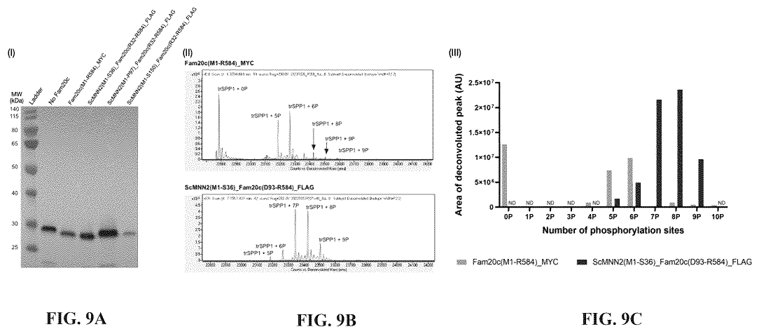

A screen for phosphorylation was carried out as follows. Supernatant samples were electrophoresed in denaturing conditions with a NuPAGE 10% Bis-Tris protein gel, transferred to a nitrocellulose blot, blocked with 5% milk in TBS-T and probed with HRP-conjugated anti-DYKDDDDK Mouse Monoclonal Antibody (“DYKDDDDK” is disclosed as SEQ ID NO: 10). Chemiluminescent detection was performed with ECL substrate kit (High Sensitivity). As phosphorylation changes the migration behavior of the protein in electrophoresis, the extent of phosphorylation was semi-quantitatively estimated. In the context of the particular assay described in this example, a downward shift of the SPP1 phosphorylation reporter construct is indicative of phosphorylation as gel migration is influenced by both protein mass and charge. This trend was confirmed by treating the phosphorylated with phosphatase in vitro (data not shown). A representative gel for the western blot-based phosphorylation screen is shown in FIG. 9A. A summary of results from the western-blot based phosphorylation screen for the panel of engineered huFam20c are summarized in Table 13.

To confirm the screen, LC-MS detection of intact isoforms was carried out to verify the number of phosphorylation sites which were occupied on TRX_SPP1 (G158-N314) when co-expressed with either engineered Fam20C or the native huFam20c control (FIG. 9B and FIG. 9C). Supernatant samples were acetone precipitated and a quadrupole time of flight (QTOF) mass analyzer (MS) was used to identify the proteins in the supernatant samples. An automatic workflow (Bioconfirm software) was applied by including the following steps: spectrum preprocessing, peak detection, deconvolution of peaks and charge state assignment. Finally, the masses and abundances of the molecular species (proteins) were reported. To demonstrate improvement in population phosphorylation, the integrated area of the deconvoluted spectral peaks was matched against the corresponding number of phosphorylation sites. LC-MS analysis was performed on a subset of engineered huFam20c constructs and the results are summarized in Table 13.

TABLE 13 provides a summary of phosphorylation data for engineered kinases.

TABLE 13
Summary of phosphorylation data for engineered kinases

| SEQ | Membrane | | | | |
|---|---|---|---|---|---|
| ID | anchoring | | | Phosphorylation | Phosphorylation |
| NO: | domain | Kinase | Construct | by WB? | by LC-MS? |
| N/A | N/A | N/A | No kinase | No | No |
| | | | expressed | | |
| 93 | Native | Fam20c | Fam20c(M1- | Not tested | Not tested |
| | Fam20c | (M1- | R584)_FLAG | | |
| | derived | R584) | | | |
| 154 | Native | Fam20c | Fam20c(M1- | Yes | Yes |
| | Fam20c | (M1- | R584)_MYC | | |
| | derived | R584) | | | |
| 94 | N/A | Fam20c | Fam20c(R32- | No | Not tested |
| | | (R32- | R584)_FLAG | | |
| | | R584) | | | |
| 95 | ScKRE2 | Fam20c | ScKRE(M1- | Yes | Yes |
| | (M1-I58) | (R32- | I58)_Fam20c(R32- | | |
| | | R584) | R584)_FLAG | | |
| 96 | ScKRE2 | Fam20c | ScKRE2(M1- | Yes | Not tested |
| | (M1-S80) | (R32- | S80)_Fam20c(R32- | | |
| | | R584) | R584)_FLAG | | |
| 97 | ScKRE2 | Fam20c | ScKRE2(M1- | Yes | Not tested |
| | (M1-D102) | (R32- | D102)_Fam20c(R32- | | |
| | | R584) | R584)_FLAG | | |
| 98 | PpKRE2 | Fam20c | PpKRE2(M1- | Yes | Not tested |
| | (M1-G31) | (R32- | G31)_Fam20c(R32- | | |
| | | R584) | R584)_FLAG | | |
| 99 | PpKRE2 | Fam20c | PpKRE2(M1- | No | Not tested |
| | (M1-D84) | (R32- | D84)_Fam20c(R32- | | |
| | | R584) | R584)_FLAG | | |
| 100 | PpKRE2 | Fam20c | PpKRE2(M1- | Yes | Not tested |
| | (M1-H150) | (R32- | H150)_Fam20c(R32- | | |
| | | R584) | R584)_FLAG | | |
| 101 | ScMNN2 | Fam20c | ScMNN2(M1- | Yes | Yes |
| | (M1-S36) | (R32- | S36)_Fam20c(R32- | | |
| | | R584) | R584)_FLAG | | |
| 102 | ScMNN2 | Fam20c | ScMNN2(M1- | No | Yes |
| | (M1-P97) | (R32- | P97)_Fam20c(R32- | | |
| | | R584) | R584)_FLAG | | |
| 103 | ScMNN2 | Fam20c | ScMNN2(M1- | No | Yes |
| | (M1-S150) | (R32- | S150)_Fam20c(R32- | | |
| | | R584) | R584)_FLAG | | |
| 104 | ScMNN1 | Fam20c | ScMNN1(M1- | Yes | Not tested |
| | (M1-A42) | (R32- | A42)_Fam20c(R32- | | |
| | | R584) | R584)_FLAG | | |
| 105 | ScMNN1 | Fam20c | ScMNN1(M1- | Yes | Not tested |
| | (M1-Q93) | (R32- | Q93)_Fam20c(R32- | | |
| | | R584) | R584)_FLAG | | |
| 106 | ScMNN1 | Fam20c | ScMNN1(M1- | Yes | Not tested |
| | (M1-G153) | (R32- | G153)_Fam20c(R32- | | |
| | | R584) | R584)_FLAG | | |
| 107 | ScMNN6 | Fam20c | ScMNN6(M1- | Yes | Yes |
| | (M1-P30) | (R32- | P30)_Fam20c(R32- | | |
| | | R584) | R584)_FLAG | | |
| 108 | ScMNN6 | Fam20c | ScMNN6(M1- | Yes | Not tested |
| | (M1-V85) | (R32- | V85)_Fam20c(R32- | | |
| | | R584) | R584)_FLAG | | |
| 109 | ScMNN6 | Fam20c | ScMNN6(M1- | Yes | Not tested |
| | (M1-E160) | (R32- | E160)_Fam20c(R32- | | |
| | | R584) | R584)_FLAG | | |
| 110 | N/A | Fam20c | Fam20c(R64- | No | Not tested |
| | | (R64- | R584)_FLAG | | |
| | | R584) | | | |
| 111 | ScKRE2 | Fam20c | ScKRE(M1- | Yes | Not tested |
| | (M1-I58) | (R64- | I58)_Fam20c(R64- | | |
| | | R584) | R584)_FLAG | | |
| 112 | ScKRE2 | Fam20c | ScKRE2(M1- | Yes | Not tested |
| | (M1-S80) | (R64- | S80)_Fam20c(R64- | | |
| | | R584) | R584)_FLAG | | |
| 113 | ScKRE2 | Fam20c | ScKRE2(M1- | Yes | Not tested |
| | (M1-D102) | (R64- | D102)_Fam20c(R64- | | |
| | | R584) | R584)_FLAG | | |
| 114 | PpKRE2 | Fam20c | PpKRE2(M1- | Yes | Not tested |
| | (M1-G31) | (R64- | G31)_Fam20c(R64- | | |
| | | R584) | R584)_FLAG | | |
| 115 | PpKRE2 | Fam20c | PpKRE2(M1- | Inconclusive | Not tested |
| | (M1-D84) | (R64- | D84)_Fam20c(R64- | | |
| | | R584) | R584)_FLAG | | |
| 116 | PpKRE2 | Fam20c | PpKRE2(M1- | Yes | Not tested |
| | (M1-H150) | (R64- | H150)_Fam20c(R64- | | |
| | | R584) | R584)_FLAG | | |
| 117 | ScMNN2 | Fam20c | ScMNN2(M1- | Yes | Not tested |
| | (M1-S36) | (R64- | S36)_Fam20c(R64- | | |
| | | R584) | R584)_FLAG | | |
| 118 | ScMNN2 | Fam20c | ScMNN2(M1- | Inconclusive | Not tested |
| | (M1-P97) | (R64- | P97)_Fam20c(R64- | | |
| | | R584) | R584)_FLAG | | |
| 119 | ScMNN2 | Fam20c | ScMNN2(M1- | Yes | Not tested |
| | (M1-S150) | (R64- | S150)_Fam20c(R64- | | |
| | | R584) | R584)_FLAG | | |
| 120 | ScMNN1 | Fam20c | ScMNN1(M1- | Yes | Not tested |
| | (M1-A42) | (R64- | A42)_Fam20c(R64- | | |
| | | R584) | R584)_FLAG | | |
| 121 | ScMNN1 | Fam20c | ScMNN1(M1- | Yes | Not tested |
| | (M1-Q93) | (R64- | Q93)_Fam20c(R64- | | |
| | | R584) | R584)_FLAG | | |
| 122 | ScMNN1 | Fam20c | ScMNN1(M1- | Yes | Not tested |
| | (M1-G153) | (R64- | G153)_Fam20c(R64- | | |
| | | R584) | R584)_FLAG | | |
| 123 | ScMNN6 | Fam20c | ScMNN6(M1- | Yes | Not tested |
| | (M1-P30) | (R64- | P30)_Fam20c(R64- | | |
| | | R584) | R584)_FLAG | | |
| 124 | ScMNN6 | Fam20c | ScMNN6(M1- | Yes | Not tested |
| | (M1-V85) | (R64- | V85)_Fam20c(R64- | | |
| | | R584) | R584)_FLAG | | |
| 125 | ScMNN6 | Fam20c | ScMNN6(M1- | Yes | Not tested |
| | (M1-E160) | (R64- | E160)_Fam20c(R64- | | |
| | | R584) | R584)_FLAG | | |
| 126 | N/A | Fam20c | Fam20c(D93- | No | Not tested |
| | | (D93- | R584)_FLAG | | |
| | | R584) | | | |
| 127 | ScKRE2 | Fam20c | ScKRE(M1- | Yes | Not tested |
| | (M1-I58) | (D93- | I58)_Fam20c(D93- | | |
| | | R584) | R584)_FLAG | | |
| 128 | ScKRE2 | Fam20c | ScKRE2(M1- | Yes | Yes |
| | (M1-S80) | (D93- | S80)_Fam20c(D93- | | |
| | | R584) | R584)_FLAG | | |
| 129 | ScKRE2 | Fam20c | ScKRE2(M1- | Yes | Not tested |
| | (M1-D102) | (D93- | D102)_Fam20c(D93- | | |
| | | R584) | R584)_FLAG | | |
| 130 | PpKRE2 | Fam20c | PpKRE2(M1- | Yes | Not tested |
| | (M1-G31) | (D93- | G31)_Fam20c(D93- | | |
| | | R584) | R584)_FLAG | | |
| 131 | PpKRE2 | Fam20c | PpKRE2(M1- | No | Not tested |
| | (M1-D84) | (D93- | D84)_Fam20c(D93- | | |
| | | R584) | R584)_FLAG | | |
| 132 | PpKRE2 | Fam20c | PpKRE2(M1- | Yes | Not tested |
| | (M1-H150) | (D93- | H150)_Fam20c(D93- | | |
| | | R584) | R584)_FLAG | | |
| 133 | ScMNN2 | Fam20c | ScMNN2(M1- | Yes | Yes |
| | (M1-S36) | (D93- | S36)_Fam20c(D93- | | |
| | | R584) | R584)_FLAG | | |
| 134 | ScMNN2 | Fam20c | ScMNN2(M1- | No | Not tested |
| | (M1-P97) | (D93- | P97)_Fam20c(D93- | | |
| | | R584) | R584)_FLAG | | |
| 135 | ScMNN2 | Fam20c | ScMNN2(M1- | No | Not tested |
| | (M1-S150) | (D93- | S150)_Fam20c(D93- | | |
| | | R584) | R584)_FLAG | | |
| 136 | ScMNN1 | Fam20c | ScMNN1(M1- | Yes | Yes |
| | (M1-A42) | (D93- | A42)_Fam20c(D93- | | |
| | | R584) | R584)_FLAG | | |
| 137 | ScMNN1 | Fam20c | ScMNN1(M1- | Yes | Not tested |
| | (M1-Q93) | (D93- | Q93)_Fam20c(D93- | | |
| | | R584) | R584)_FLAG | | |
| 138 | ScMNN1 | Fam20c | ScMNN1(M1- | Yes | Not tested |
| | (M1-G153) | (D93- | G153)_Fam20c(D93- | | |
| | | R584) | R584)_FLAG | | |
| 139 | ScMNN6 | Fam20c | ScMNN6(M1- | Yes | Not tested |
| | (M1-P30) | (D93- | P30)_Fam20c(D93- | | |
| | | R584) | R584)_FLAG | | |
| 140 | ScMNN6 | Fam20c | ScMNN6(M1- | Yes | Not tested |
| | (M1-V85) | (D93- | V85)_Fam20c(D93- | | |
| | | R584) | R584)_FLAG | | |
| 141 | ScMNN6 | Fam20c | ScMNN6(M1- | Yes | Not tested |
| | (M1-E160) | (D93- | E160)_Fam20c(D93- | | |
| | | R584) | R584)_FLAG | | |

### Example 9: Secretion of Phosphorylated Ovalbumin Co-Expressed with ScMNN2 (M1-S36)_Fam20c (D93-R584)_FLAG in Pichia pastoris

The DNA sequence for chicken ovalbumin (SERPINB14) was ordered as a DNA fragment, codon optimized for expression in Pichia pastoris. The sequence was amplified using PCR and cloned into the pPINKα-HC commercial vector (where the AOX1 promoter has been exchanged for the GAP promoter), in frame with an N-terminal alpha mating factor (α) secretion signal, an N-terminal FLAG tag and a C-terminal HiBiT tag, generating the vector pPINKα FLAG SERPINB14 HiBiT.

The pPINKα_FLAG_SERPINB14_HiBIT vector was transformed into PichiaPink™ Strain 4 (Δade2, Δprb1, Δpep4), and putative strains confirmed by colony PCR. For each strain, clones were grown overnight in YPD, diluted to an OD600 of 1.0 in fresh YPD and expressed for 72 hours at 30° C. At 0, 24, 48 and 72 hours, samples of the supernatant were taken. At 24 and 48 hours, cultures were supplemented with additional glucose to maintain growth conditions.

Supernatant samples were electrophoresed in denaturing conditions with a NuPAGE 4-12% Bis-Tris protein gel, transferred to a nitrocellulose blot, blocked with 5% milk in TBS-T and probed with HRP-conjugated Anti-DYKDDDDK Mouse Monoclonal Antibody (“DYKDDDDK” is disclosed as SEQ ID NO: 10). Chemiluminescent detection was performed with ECL substrate kit (High Sensitivity). A positive control, of a FLAG-tagged recombinant protein, was included on each blot to confirm functionality of the Western Blot. A strain with high expression of FLAG SERPINB14_HiBiT was selected and competent cells were prepared.

The pd915 vectors containing un-engineered (Fam20c (M1-R584)_MYC) and engineered (ScMNN2 (M1-S36)_Fam20c (D93-R584) FLAG) Fam20c kinases were transformed into the FLAG SERPINB14_HiBIT strain, and putative strains confirmed by colony PCR.

To confirm phosphorylation, the strains were cultured at the 200 mL scale and recombinant ovalbumin was purified from the supernatant with affinity chromatography targeting the FLAG tag. The supernatant was incubated with anti-DYKDDDDK G1 Affinity Resin at 4° C. (“DYKDDDDK” is disclosed as SEQ ID NO: 10), with gentle stirring, overnight. The resin was separated from the supernatant and washed with Pierce centrifuge columns. The target protein was eluted by competitive binding with FLAG peptide (MDYKDHDGDYKDHDIDYKDDDDK (SEQ ID NO: 438)) at 0.5 mg/mL.

To increase the sample concentration, the eluate was processed with a VivaSpin 10 kDa MWCO column and an acetone precipitation step. Samples were electrophoresed in denaturing conditions with a NuPAGE 4-12% Bis-Tris protein gel, and visualized with InstantBlue Coomassie Stain. Bands were extracted and samples for LC-MS/MS analysis were prepared using in-gel tryptic digest. Briefly, the proteins were extracted from the gel bands and enzymatically digested by trypsin into peptides. Peptides were analyzed using a mass spectrometer, Orbitrap MS, operating in MS/MS mode. MS/MS fragmentation raw data were processed using the database search engine software CHYMERIS and applying the following modifications on the peptides: phosphorylation on Ser(S)/Thr (T)/Tyr (Y), oxidation on Met (M), and carbamidomethylation of Cys. Particularly, phosphorylation was reported for each protein sequence. The software Scaffold was used to create amino acid coverage maps of the tryptic peptides sequenced by LC-MS/MS.

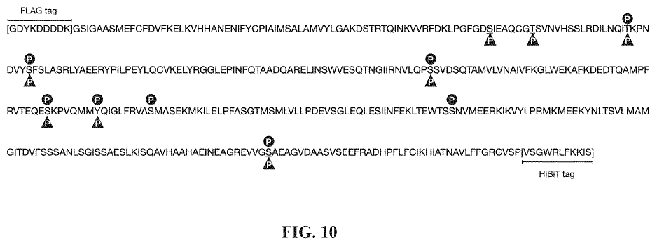

The phosphorylation of recombinant ovalbumin by engineered huFam20c and native huFam20c when co-expressed in Pichia pastoris is shown in FIG. 10. Phosphorylation sites detected in non-recombinant ovalbumin (amino acids underlined), in recombinant ovalbumin co-expressed with native huFam20c, Fam20c (M1-R584) (triangle), and in recombinant ovalbumin co-expressed with engineered huFam20c, ScMNN2M1-S36)_Fam20c (D93-R584) FLAG (circle), are annotated on the recombinant ovalbumin sequence shown in FIG. 10. The data show that native huFam20c phosphorylates recombinant ovalbumin at S69, T76, T92, S99, S165, T202, S206, and S345 (FIG. 10). The data show that engineered huFam20c, ScMNN2 (M1-S36)_Fam20c (D93-R584) FLAG, phosphorylates recombinant ovalbumin at T92, S99, S165, T202, S206, S222, S271, and S345 (FIG. 10). The data indicate that co-expression of native huFam20c and engineered huFam20c leads to distinct phosphorylation patterns of a substrate, recombinant ovalbumin.

### Example 10: Secretion of Phosphorylated Casein Co-Expressed with ScMNN2 (M1-S36)_Fam20c (D93-R584)_FLAG in Kluyveromyces lactis

The DNA sequences for bovine αS-1 casein, αS-2 casein, β-casein and κ-casein were ordered as DNA fragments, codon optimized for expression in Kluyveromyces lactis (see codon-optimized nucleic acid sequences in Table 12). The sequences were amplified using PCR and each cloned into a proprietary vector (pBDα) containing sequences with homology to the NTS2 locus flanking regions, which enable multicopy integration in the host genome. The amplified sequences were cloned in frame with sequence encoding an N-terminal alpha mating factor (α) secretion signal (SEQ ID NO: 426), generating vectors: (i) pBDα_αS1, (ii) pBDα_αS2, (iii) pBDα_β and (iv) pBDα_K. The sequence of the N-terminal alpha mating factor (α) secretion signal (SEQ ID NO: 426) (pre-Ost1+pro-alpha-factor hybrid mating signal) is

MRQVWFSWIVGLFLCFFNVSSAAPVNTTTEDETAQIPAEAVIGYSDLEG
DFDVAVLPFSNSTNNGLLFINTTIASIAAKEEGVSLDKR.

The expression cassettes for Fam20c (M1-R584) FLAG and ScMNN2 (M1-S36)_Fam20c (D93-R584)_FLAG were amplified from vectors pPINK_Fam20c (M1-R584)_FLAG and pd915_ScMNN2 (M1-S36)_Fam20c (D93-R584) FLAG respectively and each cloned into a vector containing sequences with homology to the ku80 locus flanking regions. In a second step, the GAP1 promoter from Kluyveromyces lactis (SEQ ID NO: 415) was inserted in frame with the kinase expression cassette, generating vectors: pKL_Fam20c (M1-R584)_FLAG (see open reading frame nucleic acid sequence SEQ ID NO: 361) and pKL_ScMNN2 (M1-S36)_Fam20c (D93-R584) FLAG (see open reading frame nucleic acid sequence SEQ ID NO: 406).

The vectors pBDα_αS1, pBDα_αS2, pBDα_β and pBDα_κ were co-transformed into Kluyveromyces lactis GG799 ΔKLLA0D01507g Δku80 with vectors pKL_Fam20c (M1-R584)_FLAG or pKL_ScMNN2 (M1-S36)_Fam20c (D93-R584)_FLAG, generating two putative strains expressing caseins and kinase confirmed by colony PCR.

To increase protein titre in the secretome, a fragment to knockout the protease gene, YPS1, was generated by PCR amplification. The fragment comprised a Zeocin expression cassette flanked by 500 bp sequences with homology to the YPS1 locus flanking regions. The purified PCR product was transformed into both Kluyveromyces lactis GG799 ΔKLLA0D01507g Δku80 strains expressing caseins αS1, αS2, β, κ and un-engineered (Fam20c(M1-R584) FLAG) or engineered (ScMNN2(M1-S36)_Fam20c(D93-R584) FLAG)Fam20c kinase. Putative strains were confirmed by colony PCR. For each strain, clones were diluted to an OD600 of 0.2 in fresh YPD medium and expressed for 70 hours at 30° C.

To increase the sample concentration, the supernatant samples were processed with a VivaSpin 10 kDa MWCO column. Samples were electrophoresed in denaturing conditions with a NuPAGE 12% Bis-Tris protein gel, and visualized with InstantBlue Coomassie Stain. To increase the sample concentration, the supernatant samples were processed with a VivaSpin 10 kDa MWCO column. Samples were electrophoresed in denaturing conditions with a NuPAGE 12% Bis-Tris protein gel, and visualized with InstantBlue Coomassie Stain.

Bands were extracted and samples for LC-MS/MS analysis were prepared using in-gel tryptic digest. Briefly, the proteins were extracted from the gel bands and enzymatically digested by trypsin into peptides. Peptides were analyzed using a mass spectrometer, Orbitrap MS, operating in MS/MS mode. MS/MS fragmentation raw data was processed using the database search engine software CHYMERIS and applying the following modifications on the peptides: phosphorylation on Ser(S)/Thr (T)/Tyr (Y), oxidation on Met (M), and carbamidomethylation of Cys (C). Particularly, phosphorylation was reported for each protein sequence. The software Scaffold was used to create amino acid coverage maps of the tryptic peptides sequenced by LC-MS/MS.

All four recombinant caseins, αS-1 casein (SEQ ID NO: 422), αS-2 casein (SEQ ID NO: 423), β-casein (SEQ ID NO: 424) and κ-casein (SEQ ID NO: 425), were expressed. The secretion sequence is cleaved during secretion from the yeast cell, yielding bovine αS1 casein (variant C) (no signal peptide) (SEQ ID NO: 177), bovine αS2 casein (no signal peptide) (SEQ ID NO: 178), bovine β casein (variant A2) (no signal peptide) (SEQ ID NO: 179), and bovine κ casein (variant B) (no signal peptide) (SEQ ID NO: 180).

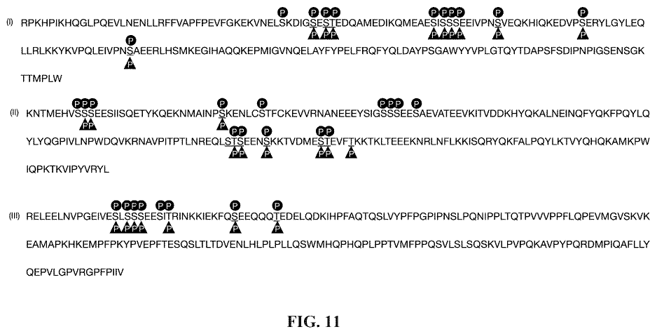

The phosphorylation of recombinant caseins by engineered huFam20c and native huFam20c when co-expressed in Kluyveromyces lactis is shown in FIG. 11. The phosphorylation status of non-recombinant αS1, αS2, β and κ-casein was tested as well. Non-recombinant caseins were obtained from Bacarel Express (Micellar Casein Concentrate Powder (MicCC85); product specification no. PS-13-01/EN (powder micellar casein concentrate of total milk protein processed by ultrafiltration and spray drying process).

The sequence coverage for non-recombinant αS1, αS2, β and κ-casein was 85, 75, 100 and 42% respectively. The sequence coverage for recombinant αS1, αS2, β and κ-casein co-expressed with Fam20c(M1-R584) was 71, 67, 93 and 36% respectively. The sequence coverage for recombinant αS1, αS2, β and κ-casein co-expressed with ScMNN2(M1-S36)_Fam20c (D93-R584) FLAG was 72, 94, 98 and 35% respectively. The data for-casein is not presented due to the low sequence coverage for this protein.

Phosphorylation sites detected in non-recombinant αS-1 casein (amino acids underlined), in recombinant αS-1 casein co-expressed with native huFam20c, Fam20c (M1-R584) (triangle), and in recombinant αS-1 casein co-expressed with engineered huFam20c, ScMNN2 (M1-S36)_Fam20c (D93-R584)_FLAG (circle), are annotated on the recombinant αS-1 casein sequence shown in FIG. 11 (I). Amino acid position numbering is relative to bovine αS-1 casein (variant C) with native signal peptide (SEQ ID NO: 416). Non-recombinant αS-1 casein obtained as described above was phosphorylated at S61, S63, T64, S90, S103, and S130. The data show that native huFam20c phosphorylates recombinant αS-1 casein at S61, S63, T64, S79, S81, S82, S83, S90, S103, and S130 (FIG. 11 (I)). The data show that engineered huFam20c, ScMNN2 (M1-S36) Fam20c (D93-R584) FLAG, phosphorylates recombinant αS-1 casein at S56, S61, S63, T64, S79, S81, S82, S83, S90, S103, and S130 (FIG. 11 (I)).

Phosphorylation sites detected in non-recombinant αS-2 casein (amino acids underlined), in recombinant αS-2 casein co-expressed with native huFam20c, Fam20c (M1-R584) (triangle), and in recombinant αS-2 casein co-expressed with engineered huFam20c, ScMNN2 (M1-S36)_Fam20c (D93-R584) FLAG (circle), are annotated on the recombinant αS-2 casein sequence shown in FIG. 11 (II). Amino acid position numbering is relative to bovine αS2 casein with native signal peptide (SEQ ID NO: 417). Non-recombinant αS-2 casein obtained as described above was phosphorylated at S46, S144, T145, S146, S150, S158, T159, T163. The data show that native huFam20c phosphorylates recombinant &S-2 casein at S24, S25, S46, T145, S146, S150, S158, T159, and T163 (FIG. 11 (II)). The data show that engineered huFam20c, ScMNN2 (M1-S36)_Fam20c (D93-R584)_FLAG, phosphorylates recombinant αS-2 casein at S23, S24, S25, S46, S52, S71, S72, S73, S76, T145, S146, S150, S158, and T159 (FIG. 11 (II)).

Phosphorylation sites detected in non-recombinant β-casein (amino acids underlined), in recombinant β-casein co-expressed with native huFam20c, Fam20c (M1-R584) (triangle), and in recombinant β-casein co-expressed with engineered huFam20c, ScMNN2 (M1-S36)_Fam20c (D93-R584) FLAG (circle), are annotated on the recombinant β-casein sequence shown in FIG. 11 (III). Amino acid position numbering is relative to bovine β casein (variant A2) with native signal peptide (SEQ ID NO: 418). Non-recombinant β-casein obtained as described above was phosphorylated at S50 and T56. The data show that native huFam20c phosphorylates recombinant β-casein at S30, S32, S33, S34, T39, S50, and T56 (FIG. 11 (III)). The data show that engineered huFam20c, ScMNN2 (M1-S36)_Fam20c (D93-R584)_FLAG, phosphorylates recombinant β-casein at S30, S32, S33, S34, S37, T39, S50, at T56 (FIG. 11 (III).

The data indicate that co-expression of native huFam20c and engineered huFam20c leads to distinct phosphorylation patterns of the substrates recombinant αS-1 casein, recombinant αS-2 casein, and recombinant β-casein.

While preferred embodiments have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the disclosure described herein may be employed in practicing the disclosure. It is intended that the following claims define the scope of the invention and that methods and structures within the scope of these claims and their equivalents be covered thereby.

1.-107. (canceled)

108. A method comprising:
(a) providing a recombinant host cell comprising:
i) a non-naturally occurring polypeptide comprising a serine/threonine kinase coupled to a heterologous anchoring domain, wherein the anchoring domain is capable of anchoring the serine/threonine kinase to an intracellular membrane of the recombinant host cell; and
ii) a protein heterologous to the recombinant host cell; and
(b) using the non-naturally occurring polypeptide to phosphorylate the protein heterologous to the recombinant host cell, thereby generating a phosphorylated protein.

109. The method of claim 108, wherein the serine/threonine kinase comprises a human kinase.

110. The method of claim 108, wherein the serine/threonine kinase comprises a Fam20c kinase.

111. The method of claim 110, wherein the Fam20c kinase comprises an amino acid sequence comprising at least 75%, 80%, 85%, 90%, 95%, or 100% sequence identity to the amino acid sequence set forth in SEQ ID NO: 7.

112. The method of claim 108, wherein the serine/threonine kinase comprises an amino acid sequence comprising at least 75%, 80%, 85%, 90%, 95%, or 100% sequence identity to the amino acid sequence set forth in SEQ ID NO: 144.

113. The method of claim 108, wherein the recombinant host cell comprises a fungal cell.

114. The method of claim 113, wherein the fungal cell comprises Aspergillus, Candida, Fusarium, Hansenula, Kluyveromyces, Pichia, Penicillium, Saccharomyces, Tetrahymena, Trichoderma, Yarrowia, Zygosaccharomyces, or any combination thereof.

115. The method of claim 113, wherein the fungal cell comprises Pichia pastoris.

116. The method of claim 113, wherein the fungal cell comprises K. lactis.

117. The method of claim 108, wherein the protein heterologous to the recombinant host cell comprises a secretory protein.

118. The method of claim 117, wherein the secretory protein comprises osteopontin.

119. The method of claim 117, wherein the secretory protein is a human secretory protein.

120. The method of claim 108, wherein the protein heterologous to the recombinant host cell comprises an amino acid sequence comprising at least 75%, 80%, 85%, 90%, 95%, or 100% sequence identity to the amino acid sequence set forth in SEQ ID NO: 5.

121. The method of claim 108, wherein the protein heterologous to the recombinant host cell comprises an amino acid sequence comprising at least 95% sequence identity to the amino acid sequence set forth in SEQ ID NO: 5.

122. The method of claim 108, wherein the anchoring domain comprises a Golgi retention sequence.

123. The method of claim 108, further comprising harvesting the phosphorylated protein from the recombinant host cell, thereby generating a harvested phosphorylated protein.

124. The method of claim 123, further comprising using the harvested phosphorylated protein for manufacture of a food product.

125. The method of claim 124, wherein the food product is a dairy product or a dairy substitute.

126. A non-naturally occurring polypeptide comprising a serine/threonine kinase coupled to a heterologous domain capable of anchoring the serine/threonine kinase to an intracellular membrane of a cell.

127. A composition comprising phosphorylated non-naturally occurring secretory polypeptides, wherein the phosphorylated non-naturally occurring secretory polypeptides comprise a higher level of phosphorylation than corresponding non-naturally occurring secretory polypeptides phosphorylated in a recombinant host cell using Fam20c kinase without a heterologous anchoring domain, wherein the phosphorylated non-naturally occurring secretory polypeptides comprise a osteopontin polypeptide or a casein polypeptide.


SEQUENCE LISTING (442 sequences)

SEQ ID NO: 1 - protein, 579 aa, Mus sp.
MKMILVRRFRVLILVVFLLACALHIAVDLLPKLDRRATRSSGEPGCSCAQPAAEAAGPGWAQARSRPGESAGGDAGWPNKHTLRILQDFSSDPASNLTSHSLEKLPSAAEPVDHAPRGQEPRSPPPRDPAHRPLLRDPGPRPRVPPPGPSGDGSLLAKLFEHPLYQGAVPPLTEDDVLFNVNSDIRFNPKAAENPDWPHEGAEGAEFLPTGEAAVNLYPNWLKFHIGINRYELYSRHNPAIDALLRDLGSQKITSVAMKSGGTQLKLIMTFQNYGQALFKPMKQTREQETPPDFFYFSDYERHNAEIAAFHLDRILDFRRVPPVAGRMINMTKEIRDVTRDKKLWRTFFVSPANNICFYGECSYYCSTEHALCGRPDQIEGSLAAFLPDLSLAKRKTWRNPWRRSYHKRKKAEWEVDPDYCEEVKQTPPYDSGHRILDIMDMTVFDFLMGNMDRHHYETFEKFGNETFIIHLDNGRGFGKYSHDELSILAPLHQCCRIRRSTYLRLQLLAKEEHKLSLLMAESLQHDKVAPVLYQLHLEALDRRLRIVLQAVRDCVEKDGLSSVVEDDLATEHRASTER

SEQ ID NO: 2 - protein, 586 aa, Bos sp.
MKMILVRRFRVLILMAFLAACALHLVLDLLPKLERSAARPSGEPGCSCAQPAAEAAAPGWAQARGHPGGELEAAASAAGDAGWPNKHTLRILQDFSSDPSSNLTSHSLEKLPPAAEAAEGAPPGQDPGVRRPPDPAHRPLPRDPGPRGPVLPPGLSGDGSLLTRLFQHPLYQVPIPPLTEGDVLFNVNSDIRFNPKAATAENPDWPHEGPEDEFLPTGEAAVDSYPNWLKFHIGINRYELYSRHNPAVGALLQDLGTQKITSVAMKSGGTQLKLIMTFQNYGQALFKPMKQTREQETPPDFFYFSDYERHNAEIAAFHLDRILDFRRVPPVAGRLVNMTKEIRDVTRDKKLWRTFFISPANNVCFYGECSYYCSTEHALCGKPDQIEGSLAAFLPDLALAKRKTWRNPWRRSYHKRKKAEWEVDPDYCEEVRQTPPYDSSHRLLDVMDMTIFDFLMGNMDRHHYETFEKFGNETFIIHLDNGRGFGKHSHDELSILVPLQQCCRIRRSTYLRLQLLAQEEHRLSLLMAEALRADRVAPVLFQPHLEALDRRLRIVLRAVGDCVEKDGLHSVVEDDLGPEHRAAAGR

SEQ ID NO: 3 - protein, 141 aa, unidentified
MDMTIFDFLMGNMDRHHYETFEKFGNNTFIIHLDNGRGFGKYSHDELSILVPLNQCCRIRKSTYLRLQLLAKEEYKLSQLMEESLLEDKIAPILYQLHLEAMDRRLRIVLKAIRDCIEKGSYNKVVENDFASPRSTVTTER

SEQ ID NO: 4 - protein, 199 aa, synthetic construct
RPKHPIKHQGLPQEVLNENLLRFFVAPFPEVFGKEKVNELSKDIGSESTEDQAMEDIKQMEAESISSSEEIVPNSVEQKHIQKEDVPSERYLGYLEQLLRLKKYKVPQLEIVPNSAEERLHSMKEGIHAQQKEPMIGVNQELAYFYPELFRQFYQLDAYPSGAWYYVPLGTQYTDAPSFSDIPNPIGSENSEKTTMPLW

SEQ ID NO: 5 - protein, 298 aa, synthetic construct
IPVKQADSGSSEEKQLYNKYPDAVATWLNPDPSQKQNLLAPQNAVSSEETNDFKQETLPSKSNESHDHMDDMDDEDDDDHVDSQDSIDSNDSDDVDDTDDSHQSDESHHSDESDELVTDFPTDLPATEVFTPVVPTVDTYDGRGDSVVYGLRSKSKKFRRPDIQYPDATDEDITSHMESEELNGAYKAIPVAQDLNAPSDWDSRGKDSYETSQLDDQSAETHSHKQSRLYKRKANDESNEHSDVIDSQELSKVSREFHSHEFHSHEDMLVVDPKSKEEDKHLKFRISHELDSASSEVN

SEQ ID NO: 6 - protein, 252 aa, unidentified
MVNMTKEIRDVTRDKKLWRTFFISPANNICFYGECSYYCSTEHALCGKPDQIEGSLAAFLPDLSLAKRKTWRNPWRRSYHKRKKAEWEVDPDYCEEVKQTPPYDSGHRVLDIMDMTIFDFLMGNMDRHHYETFEKFGNETFIIHLDNGRGFGKYSHDELSILVPLQQCCRIRKSTYLRLQLLAKEEYKLSLLMAESLQRDKVAPVLYRLHLEALDRRLRIVLQAVRDCIEKDGVHSVVEDDLDTEHRASPER

SEQ ID NO: 7 - protein, 584 aa, Homo sapiens
MKMMLVRRFRVLILMVFLVACALHIALDLLPRLERRGARPSGEPGCSCAQPAAEVAAPGWAQVRGRPGEPPAASSAAGDAGWPNKHTLRILQDFSSDPSSNLSSHSLEKLPPAAEPAERALRGRDPGALRPHDPAHRPLLRDPGPRRSESPPGPGGDASLLARLFEHPLYRVAVPPLTEEDVLFNVNSDTRLSPKAAENPDWPHAGAEGAEFLSPGEAAVDSYPNWLKFHIGINRYELYSRHNPAIEALLHDLSSQRITSVAMKSGGTQLKLIMTFQNYGQALFKPMKQTREQETPPDFFYFSDYERHNAEIAAFHLDRILDFRRVPPVAGRMVNMTKEIRDVTRDKKLWRTFFISPANNICFYGECSYYCSTEHALCGKPDQIEGSLAAFLPDLSLAKRKTWRNPWRRSYHKRKKAEWEVDPDYCEEVKQTPPYDSSHRILDVMDMTIFDFLMGNMDRHHYETFEKFGNETFIIHLDNGRGFGKYSHDELSILVPLQQCCRIRKSTYLRLQLLAKEEYKLSLLMAESLRGDQVAPVLYQPHLEALDRRLRVVLKAVRDCVERNGLHSVVDDDLDTEHRAASAR

SEQ ID NO: 8 - protein, 298 aa, Syrrhaptes sp.
MQTREQETPPDFFYFSDYERHNAEIAAFHLDRILDFRRVPPVAGRLVNMTREIRDVTRDKKLWRTFFISPANNICFYGECSYYCSTEHALCGKPDQIEGSLAAFLPDLSLAKRKTWRNPWRRSYHKRKKAEWEVDPDYCEEVKQTPPYDSGTRILDIMDMTVFDFLMGNMDRHHYETFEKFGNETFIIHLDNGRGFGKYSHDELSILVPLNQCCRIRKSTYLRLQLLAKEEYKLSVLMKESLLKDKIAPILYQPHLEAMDRRLRIVLKAISDCIEKDGYINVVENDFNTDVNTVTTER

SEQ ID NO: 9 - protein, 287 aa, Homo sapiens
KMMLVRRFRVLILMVFLVACALHIALDLLPRLERRGARPSGEPGCSCAQPAAEVAAPGWAQVRGRPGEPPAASSAAGDAGWPNKHTLRILQDFSSDPSSNLSSHSLEKLPPAAEPAERALRGRDPGALRPHDPAHRPLLRDPGPRRSESPPGPGGDASLLARLFEHPLYRVAVPPLTEEDVLFNVNSDTRLSPKAAENPDWPHAGAEGAEFLSPGEAAVDSYPNWLKFHIGINRYELYSRHNPAIEALLHDLSSQRITSVAMKSGGTQLKLIMTFQNYGQALFKPMK

SEQ ID NO: 10 - protein, 8 aa, synthetic construct
DYKDDDDK

SEQ ID NO: 11 - protein, 98 aa, synthetic construct
GSLQEEKPKEGVKTENDHINLKVAGQDGSVVQFKIKRHTPLSKLMKAYCERQGLSMRQIRFRFDGQPINETDTPAQLEMEDEDTIDVFQQQTGGEFDP

SEQ ID NO: 12 - protein, 108 aa, synthetic construct
SDKIIHLTDDSFDTDVLKADGAILVDFWAEWCGPCKMIAPILDEIADEYQGKLTVAKLNIDQNPGTAPKYGIRGIPTLLLFKNGEVAATKVGALSKGQLKEFLDANLA

SEQ ID NO: 13 - protein, 395 aa, synthetic construct
KIKTGARILALSALTTMMFSASALAKIEEGKLVIWINGDKGYNGLAEVGKKFEKDTGIKVTVEHPDKLEEKFPQVAATGDGPDIIFWAHDRFGGYAQSGLLAEITPDKAFQDKLYPFTWDAVRYNGKLIAYPIAVEALSLIYNKDLLPNPPKTWEEIPALDKELKAKGKSALMFNLQEPYFTWPLIAADGGYAFKYENGKYDIKDVGVDNAGAKAGLTFLVDLIKNKHMNADTDYSIAEAAFNKGETAMTINGPWAWSNIDTSKVNYGVTVLPTFKGQPSKPFVGVLSAGINAASPNKELAKEFLENYLLTDEGLEAVNKDKPLGAVALKSYEEELAKDPRIAATMENAQKGEIMPNIPQMSAFWYAVRTAVINAASGRQTVDEALKDAQTRITK

SEQ ID NO: 14 - protein, 109 aa, synthetic construct
FCTFFEKHHRKWDILLEKSTGVMEAMKVTSEEKEQLSTAIDRMNEGLDAFIQLYNESEIDEPLIQLDDDTAELMKQARDMYGQEKLNEKLNTIIKQILSISVSEEGEKE

SEQ ID NO: 15 - protein, 494 aa, synthetic construct
NKEILAVVEAVSNEKALPREKIFEALESALATATKKKYEQEIDVRVQIDRKSGDFDTFRRWLVVDEVTQPTKEITLEAARYEDESLNLGDYVEDQIESVTFDRITTQTAKQVIVQKVREAERAMVVDQFREHEGEIITGVVKKVNRDNISLDLGNNAEAVILREDMLPRENFRPGDRVRGVLYSVRPEARGAQLFVTRSKPEMLIELFRIEVPEIGEEVIEIKAAARDPGSRAKIAVKTNDKRIDPVGACVGMRGARVQAVSTELGGERIDIVLWDDNPAQFVINAMAPADVASIVVDEDKHTMDIAVEAGNLAQAIGRNGQNVRLASQLSGWELNVMTVDDLQAKHQAEAHAAIDTFTKYLDIDEDFATVLVEEGFSTLEELAYVPMKELLEIEGLDEPTVEALRERAKNALATIAQAQEESLGDNKPADDLLNLEGVDRDLAFKLAARGVCTLEDLAEQGIDDLADIEGLTDEKAGALIMAARNICWFGDEA

SEQ ID NO: 16 - protein, 107 aa, synthetic construct
GDYKDDDDKGSLQEEKPKEGVKTENDHINLKVAGQDGSVVQFKIKRHTPLSKLMKAYCERQGLSMRQIRFRFDGQPINETDTPAQLEMEDEDTIDVFQQQTGGEFDP

SEQ ID NO: 17 - protein, 118 aa, synthetic construct
GDYKDDDDKMSDKIIHLTDDSFDTDVLKADGAILVDFWAEWCGPCKMIAPILDEIADEYQGKLTVAKLNIDQNPGTAPKYGIRGIPTLLLFKNGEVAATKVGALSKGQLKEFLDANLA

SEQ ID NO: 18 - protein, 405 aa, synthetic construct
GDYKDDDDKMKIKTGARILALSALTTMMFSASALAKIEEGKLVIWINGDKGYNGLAEVGKKFEKDTGIKVTVEHPDKLEEKFPQVAATGDGPDIIFWAHDRFGGYAQSGLLAEITPDKAFQDKLYPFTWDAVRYNGKLIAYPIAVEALSLIYNKDLLPNPPKTWEEIPALDKELKAKGKSALMFNLQEPYFTWPLIAADGGYAFKYENGKYDIKDVGVDNAGAKAGLTFLVDLIKNKHMNADTDYSIAEAAFNKGETAMTINGPWAWSNIDTSKVNYGVTVLPTFKGQPSKPFVGVLSAGINAASPNKELAKEFLENYLLTDEGLEAVNKDKPLGAVALKSYEEELAKDPRIAATMENAQKGEIMPNIPQMSAFWYAVRTAVINAASGRQTVDEALKDAQTRITK

SEQ ID NO: 19 - protein, 119 aa, synthetic construct
GDYKDDDDKMFCTFFEKHHRKWDILLEKSTGVMEAMKVTSEEKEQLSTAIDRMNEGLDAFIQLYNESEIDEPLIQLDDDTAELMKQARDMYGQEKLNEKLNTIIKQILSISVSEEGEKE

SEQ ID NO: 20 - protein, 504 aa, synthetic construct
GDYKDDDDKMNKEILAVVEAVSNEKALPREKIFEALESALATATKKKYEQEIDVRVQIDRKSGDFDTFRRWLVVDEVTQPTKEITLEAARYEDESLNLGDYVEDQIESVTFDRITTQTAKQVIVQKVREAERAMVVDQFREHEGEIITGVVKKVNRDNISLDLGNNAEAVILREDMLPRENFRPGDRVRGVLYSVRPEARGAQLFVTRSKPEMLIELFRIEVPEIGEEVIEIKAAARDPGSRAKIAVKTNDKRIDPVGACVGMRGARVQAVSTELGGERIDIVLWDDNPAQFVINAMAPADVASIVVDEDKHTMDIAVEAGNLAQAIGRNGQNVRLASQLSGWELNVMTVDDLQAKHQAEAHAAIDTFTKYLDIDEDFATVLVEEGFSTLEELAYVPMKELLEIEGLDEPTVEALRERAKNALATIAQAQEESLGDNKPADDLLNLEGVDRDLAFKLAARGVCTLEDLAEQGIDDLADIEGLTDEKAGALIMAARNICWFGDEA

SEQ ID NO: 21 - protein, 4 aa, unidentified
HDEL

SEQ ID NO: 22 - protein, 4 aa, unidentified
KDEL

SEQ ID NO: 23 - protein, 34 aa, synthetic construct
MLISKSKMFKTFWILTSIVLLASATVDISKLQEF

SEQ ID NO: 24 - protein, 85 aa, synthetic construct
MLISKSKMFKTFWILTSIVLLASATVDISKLQEFEEYQKFTNESLLWAPYRSNCYFGMRPRYVHESPLIMGIMWFNSLSQDGLHS

SEQ ID NO: 25 - protein, 137 aa, synthetic construct
MLISKSKMFKTFWILTSIVLLASATVDISKLQEFEEYQKFTNESLLWAPYRSNCYFGMRPRYVHESPLIMGIMWFNSLSQDGLHSLRHFATPQDKLQKYGWEVYDPRIGGKEVFIDEKNNLNLTVYFVKSKNGENWS

SEQ ID NO: 26 - protein, 30 aa, synthetic construct
MKNSVGISIATIVAIIAAIYYVPWYEHFER

SEQ ID NO: 27 - protein, 82 aa, synthetic construct
MKNSVGISIATIVAIIAAIYYVPWYEHFERKSPGAGEMRDRIESMFLESWRDYSKHGWGYDVYGPIEHTSHNMPRGNQPLGW

SEQ ID NO: 28 - protein, 143 aa, synthetic construct
MKNSVGISIATIVAIIAAIYYVPWYEHFERKSPGAGEMRDRIESMFLESWRDYSKHGWGYDVYGPIEHTSHNMPRGNQPLGWIIVDSVDTLMLMYNSSTLYKSEFEAEIQRSEHWINDVLDFDIDAEVNVFETTIRMLGGLLS

SEQ ID NO: 29 - protein, 51 aa, synthetic construct
ANTIHIIKLPLNYANYTSMKQKISKFFTNFILIVLLSYILQFSYKHNLHSM

SEQ ID NO: 30 - protein, 103 aa, synthetic construct
ANTIHIIKLPLNYANYTSMKQKISKFFTNFILIVLLSYILQFSYKHNLHSMLFNYAKDNFLTKRDTISSPYVVDEDLHQTTLFGNHGTKTSVPSVDSIKVHGV

SEQ ID NO: 31 - protein, 142 aa, synthetic construct
ANTIHIIKLPLNYANYTSMKQKISKFFTNFILIVLLSYILQFSYKHNLHSMLFNYAKDNFLTKRDTISSPYVVDEDLHQTTLFGNHGTKTSVPSVDSIKVHGVHETSSVNGTEVLCTESNIINTGGAEFEITNATFREIDDA

SEQ ID NO: 32 - protein, 29 aa, synthetic construct
PRKIFNYFILTVFMAILAIVLQWSIENGH

SEQ ID NO: 33 - protein, 50 aa, synthetic construct
MAKADGSLLYYNPHNPPRRYYFYMAIFAVSVICVLYGPSQQLSSPKIDYD

SEQ ID NO: 34 - protein, 80 aa, synthetic construct
MAKADGSLLYYNPHNPPRRYYFYMAIFAVSVICVLYGPSQQLSSPKIDYDPLTLRSLDLKTLEAPSQLSPGTVEDNLRRQ

SEQ ID NO: 35 - protein, 134 aa, synthetic construct
MAKADGSLLYYNPHNPPRRYYFYMAIFAVSVICVLYGPSQQLSSPKIDYDPLTLRSLDLKTLEAPSQLSPGTVEDNLRRQLEFHFPYRSYEPFPQHIWQTWKVSPSDSSFPKNFKDLGESWLQRSPNYDHFVIP

SEQ ID NO: 36 - protein, 40 aa, synthetic construct
MSLSLVSYRLRKNPWVNIFLPVLAIFLIYIIFFQRDQSLL

SEQ ID NO: 37 - protein, 91 aa, synthetic construct
MSLSLVSYRLRKNPWVNIFLPVLAIFLIYIIFFQRDQSLLGLNGQSISQHKWAHEKENTFYFPFTKKYKMPKYSYKKKSGWLFNDHVEDII

SEQ ID NO: 38 - protein, 154 aa, synthetic construct
MSLSLVSYRLRKNPWVNIFLPVLAIFLIYIIFFQRDQSLLGLNGQSISQHKWAHEKENTFYFPFTKKYKMPKYSYKKKSGWLFNDHVEDIIPEGHIAHYDLNKLHSTSEAAVNKEHILILTPMQTFHQQYWDNLLQLNYPRELIELGFITPRTA

SEQ ID NO: 39 - protein, 93 aa, synthetic construct
MGMFFNLRSNIKKKAMDNGLSLPISRNGSSNNIKDKRSEHNSNSLKGKYRYQPRSTPSKFQLTVSITSLIIIAVLSLYLFISFLSGMGIGVST

SEQ ID NO: 40 - protein, 98 aa, synthetic construct
MGMFFNLRSNIKKKAMDNGLSLPISRNGSSNNIKDKRSEHNSNSLKGKYRYQPRSTPSKFQLTVSITSLIIIAVLSLYLFISFLSGMGIGVSTQNGRS

SEQ ID NO: 41 - protein, 203 aa, synthetic construct
MGMFFNLRSNIKKKAMDNGLSLPISRNGSSNNIKDKRSEHNSNSLKGKYRYQPRSTPSKFQLTVSITSLIIIAVLSLYLFISFLSGMGIGVSTQNGRSLLGSSKSSENYKTIDLEDEEYYDYDFEDIDPEVISKFDDGVQHYLISQFGSEVLTPKDDEKYQRELNMLFDSTVEEYDLSNFEGAPNGLETRDHILLCIPLRNAA

SEQ ID NO: 42 - protein, 60 aa, synthetic construct
MKYNNRKLSFNPTTVSIAGTLLTVFFLTRLVLSFFSISLFQLVTFQGIFKPYVPDFKNTP

SEQ ID NO: 43 - protein, 82 aa, synthetic construct
MKYNNRKLSFNPTTVSIAGTLLTVFFLTRLVLSFFSISLFQLVTFQGIFKPYVPDFKNTPSVEFYDLRNYQGNKDGWQQGDR

SEQ ID NO: 44 - protein, 105 aa, synthetic construct
MKYNNRKLSFNPTTVSIAGTLLTVFFLTRLVLSFFSISLFQLVTFQGIFKPYVPDFKNTPSVEFYDLRNYQGNKDGWQQGDRILFCVPLRDASEHLPMFFNHLNT

SEQ ID NO: 45 - protein, 34 aa, synthetic construct
MAKTTKRASSFRRLMIFAIIALISLAFGVRYLFH

SEQ ID NO: 46 - protein, 84 aa, synthetic construct
MAKTTKRASSFRRLMIFAIIALISLAFGVRYLFHNSNATDLQKILQNLPKEISQSINSANNIQSSDSDLVQHFESLAQEIRHQQ

SEQ ID NO: 47 - protein, 145 aa, synthetic construct
MAKTTKRASSFRRLMIFAIIALISLAFGVRYLFHNSNATDLQKILQNLPKEISQSINSANNIQSSDSDLVQHFESLAQEIRHQQEVQAKQFDKQRKILEKKIQDLKQTPPEATLRERIAMTFPYDSHVKFPAFIWQTWSNDEGPE

SEQ ID NO: 48 - protein, 73 aa, synthetic construct
MSSVPYNSQLPISNHLEYDEDEKKSRGSKLGLKYKMIYWRKTLCSSLARWRKLILLISLALFLFIWISDSTIS

SEQ ID NO: 49 - protein, 121 aa, synthetic construct
MSSVPYNSQLPISNHLEYDEDEKKSRGSKLGLKYKMIYWRKTLCSSLARWRKLILLISLALFLFIWISDSTISRNPSTTSFQGQNSNDNKLSNTGSSINSKRYVPPYSKRSRWSFWNQDPR

SEQ ID NO: 50 - protein, 172 aa, synthetic construct
MSSVPYNSQLPISNHLEYDEDEKKSRGSKLGLKYKMIYWRKTLCSSLARWRKLILLISLALFLFIWISDSTISRNPSTTSFQGQNSNDNKLSNTGSSINSKRYVPPYSKRSRWSFWNQDPRIVIILAANEGGGVLRWKNEQEWAIEGISIENKKAYAKRHGYALTIKDLTTS

SEQ ID NO: 51 - protein, 52 aa, synthetic construct
MAIKPRTKGKTYSSRSVGSQWFNRLGFKQNKYGTCKFLSIITAFVFILYFFS

SEQ ID NO: 52 - protein, 101 aa, synthetic construct
MAIKPRTKGKTYSSRSVGSQWFNRLGFKQNKYGTCKFLSIITAFVFILYFFSNRFYPISRSAGASYSPSHGLYINEIPASSRLIYPHVEHVPVLKQMTVRG

SEQ ID NO: 53 - protein, 165 aa, synthetic construct
MAIKPRTKGKTYSSRSVGSQWFNRLGFKQNKYGTCKFLSIITAFVFILYFFSNRFYPISRSAGASYSPSHGLYINEIPASSRLIYPHVEHVPVLKQMTVRGLYITRLEVDGSKRLILKPEENALTDEEKKKTTDQILLVKHSFLDHGKLVYRKSNDAPEVVVVTL

SEQ ID NO: 54 - protein, 58 aa, synthetic construct
MALFLSKRLLRFTVIAGAVIVLLLTLNSNSRTQQYIPSSISAAFDFTSGSISPEQQVI

SEQ ID NO: 55 - protein, 80 aa, synthetic construct
MALFLSKRLLRFTVIAGAVIVLLLTLNSNSRTQQYIPSSISAAFDFTSGSISPEQQVISEENDAKKLEQSALNSEASEDS

SEQ ID NO: 56 - protein, 102 aa, synthetic construct
MALFLSKRLLRFTVIAGAVIVLLLTLNSNSRTQQYIPSSISAAFDFTSGSISPEQQVISEENDAKKLEQSALNSEASEDSEAMDEESKALKAAAEKADAPID

SEQ ID NO: 57 - protein, 72 aa, synthetic construct
MAGATRINSRVVRFAIFASILVLLGFILSRGSATSYSLPSGLTSDTSQSTGSSPKSESKPSSQGSSGATELK

SEQ ID NO: 58 - protein, 112 aa, synthetic construct
MAGATRINSRVVRFAIFASILVLLGFILSRGSATSYSLPSGLTSDTSQSTGSSPKSESKPSSQGSSGATELKKTYTTDGKEKATFVSLARNSDVWSLASSIRHVEDRFNHKF

SEQ ID NO: 59 - protein, 196 aa, synthetic construct
MAGATRINSRVVRFAIFASILVLLGFILSRGSATSYSLPSGLTSDTSQSTGSSPKSESKPSSQGSSGATELKKTYTTDGKEKATFVSLARNSDVWSLASSIRHVEDRFNHKFHYDWVFLNDEEFSDEFKRVTSALTSGKAKYGLIPKEHWSFPEWIDKERAAKTRKEMAAKKVIYGDSISYRHMCRFESGFFFRHE

SEQ ID NO: 60 - protein, 30 aa, synthetic construct
MMRARLSLERVNLSFITSVFLASVAVLFIS

SEQ ID NO: 61 - protein, 75 aa, synthetic construct
MMRARLSLERVNLSFITSVFLASVAVLFISLEMPKVLARDRQILKLKLGFMGSGLQKGSLETSGNIENTESNINS

SEQ ID NO: 62 - protein, 163 aa, synthetic construct
MMRARLSLERVNLSFITSVFLASVAVLFISLEMPKVLARDRQILKLKLGFMGSGLQKGSLETSGNIENTESNINSQTTQHIGTIGASNERANATFYTLCRNEELYQMLETVQNYEDRFNSKFKYDWVFLNDYPFTDEFKRVISHAISGEAKFGQVPASHWRFP

SEQ ID NO: 63 - protein, 31 aa, synthetic construct
MVHIGFRSLKAVFILALSSLILYGIVTTFDG

SEQ ID NO: 64 - protein, 84 aa, synthetic construct
MVHIGFRSLKAVFILALSSLILYGIVTTFDGSRASRYQPPYVNHSQDPLYHSGNSYNRENATFVTLCRNEDLYSIIQSIKKVED

SEQ ID NO: 65 - protein, 150 aa, synthetic construct
MVHIGFRSLKAVFILALSSLILYGIVTTFDGSRASRYQPPYVNHSQDPLYHSGNSYNRENATFVTLCRNEDLYSIIQSIKKVEDRFNNKFAYDWVFLNEVPFTDEFKERTSVLISGQAKYGLIPKEHWSYPDYIDQERAAESRRQLEDQH

SEQ ID NO: 66 - protein, 39 aa, synthetic construct
MAKIMIPASKQPVYKKLGLLLVAVFTVYVFFHGAQYARG

SEQ ID NO: 67 - protein, 155 aa, synthetic construct
MAKIMIPASKQPVYKKLGLLLVAVFTVYVFFHGAQYARGSAPSPKYSTVLSSGSGYKYSKVELPKYTGPREKATFVTLVRNRDLYSLAESIKSVEDRFNSKFNYDWVFLNDEEFTDEFKNVTSALVSGTTKYGVIPKEHWSFPEWIDEEKAAQVR

SEQ ID NO: 68 - protein, 40 aa, synthetic construct
MQICKVFLTQVKKLLFVSLLFCLIAQTCWLALVPYQRQLS

SEQ ID NO: 69 - protein, 100 aa, synthetic construct
MQICKVFLTQVKKLLFVSLLFCLIAQTCWLALVPYQRQLSLDSYFFRRSREVSSRYDFTRRRHMNQTLKLSSNTYNDEPLNKTKGIKNQRENATLLMLVR

SEQ ID NO: 70 - protein, 170 aa, synthetic construct
MQICKVFLTQVKKLLFVSLLFCLIAQTCWLALVPYQRQLSLDSYFFRRSREVSSRYDFTRRRHMNQTLKLSSNTYNDEPLNKTKGIKNQRENATLLMLVRNWELSGALRSMRSLEDRFNKNYQYDWTFLNDVPFDQEFIEATTAMASGRTQYALIPAEDWNRPSWINETL

SEQ ID NO: 71 - protein, 36 aa, synthetic construct
MLLTKRFSKLFKLTFIVLILCGLFVITNKYMDENTS

SEQ ID NO: 72 - protein, 97 aa, synthetic construct
MLLTKRFSKLFKLTFIVLILCGLFVITNKYMDENTSVKEYKEYLDRYVQSYSNKYSSSSDAASADDSTPLRDNDEAGNEKLKSFYNNVFNFLMVDSP

SEQ ID NO: 73 - protein, 150 aa, synthetic construct
MLLTKRFSKLFKLTFIVLILCGLFVITNKYMDENTSVKEYKEYLDRYVQSYSNKYSSSSDAASADDSTPLRDNDEAGNEKLKSFYNNVFNFLMVDSPKGSTAKQYNEACLLKGDIGDRPDHYKDLYKLSAKELSKCLELSPDEVASLTKS

SEQ ID NO: 74 - protein, 35 aa, synthetic construct
MLIRLKKRKILQVIVSAVVLILFFCSVHNDVSSSW

SEQ ID NO: 75 - protein, 85 aa, synthetic construct
MLIRLKKRKILQVIVSAVVLILFFCSVHNDVSSSWLYGKKLRLPVLTRSNLKNNFYTTLVQAIVENKPADSSPDLSKLHGAEGCS

SEQ ID NO: 76 - protein, 150 aa, synthetic construct
MLIRLKKRKILQVIVSAVVLILFFCSVHNDVSSSWLYGKKLRLPVLTRSNLKNNFYTTLVQAIVENKPADSSPDLSKLHGAEGCSFANNVAAHDSGHDSDLSYESLSKCYNLNKTVQESLREVHSKFTDTLSGKLNFSIPQREALFSGSE

SEQ ID NO: 77 - protein, 36 aa, synthetic construct
MAKGGSLYIVGIFLPIWTFMIYIFGKELFLIRKYQK

SEQ ID NO: 78 - protein, 90 aa, synthetic construct
MAKGGSLYIVGIFLPIWTFMIYIFGKELFLIRKYQKIDSTYTALSQRVKEQYDTSRRRNYFPKVKLSRNSYDDYTLNYTRQNDSDSFHLR

SEQ ID NO: 79 - protein, 150 aa, synthetic construct
MAKGGSLYIVGIFLPIWTFMIYIFGKELFLIRKYQKIDSTYTALSQRVKEQYDTSRRRNYFPKVKLSRNSYDDYTLNYTRQNDSDSFHLRENATILMLVRNSELEGALDSMRSLEDRFNNKYHYDWTFLNDVPFDQDFIEATTSMASGKT

SEQ ID NO: 80 - protein, 42 aa, synthetic construct
MLALRRFILNQRSLRSCTIPILVGALIIILVLFQLVTHRNDA

SEQ ID NO: 81 - protein, 93 aa, synthetic construct
MLALRRFILNQRSLRSCTIPILVGALIIILVLFQLVTHRNDALIRSSNVNSTNKKTLKDADPKVLIEAFGSPEVDPVDTIPVSPLELVPFYDQ

SEQ ID NO: 82 - protein, 153 aa, synthetic construct
MLALRRFILNQRSLRSCTIPILVGALIIILVLFQLVTHRNDALIRSSNVNSTNKKTLKDADPKVLIEAFGSPEVDPVDTIPVSPLELVPFYDQSIDTKRSSSWLINKKGYYKHFNELSLTDRCKFYFRTLYTLDDEWTNSVKKLEYSINDNEG

SEQ ID NO: 83 - protein, 30 aa, synthetic construct
MHVLLSKKIARFLLISFVFVLALMVTINHP

SEQ ID NO: 84 - protein, 75 aa, synthetic construct
MHVLLSKKIARFLLISFVFVLALMVTINHPKTKQMSEQYVTPYLPKSLQPIAKISAEEQRRIQSEQEEAELKQSL

SEQ ID NO: 85 - protein, 160 aa, synthetic construct
MHVLLSKKIARFLLISFVFVLALMVTINHPKTKQMSEQYVTPYLPKSLQPIAKISAEEQRRIQSEQEEAELKQSLEGEAIRNATVNAIKEKIKSYGGNETTLGFMVPSYINHRGSPPKACFVSLITERDSMTQILQSIDEVQVKFNKNFAYPWVFISQGE

SEQ ID NO: 86 - DNA, 939 bp, synthetic construct
  source 1..939: mol_type = other DNA
gatctaacatccaaagacgaaaggttgaatgaaacctttttgccatccgacatccacaggtccattctcacacataagtgccaaacgcaacaggaggggatacactagcagcagaccgttgcaaacgcaggacctccactcctcttctcctcaacacccacttttgccatcgaaaaaccagcccagttattgggcttgattggagctcgctcattccaattccttctattaggctactaacaccatgactttattagcctgtctatcctggcccccctggcgaggttcatgtttgtttatttccgaatgcaacaagctccgcattacacccgaacatcactccagatgagggctttctgagtgtggggtcaaatagtttcatgttccccaaatggcccaaaactgacagtttaaacgctgtcttggaacctaatatgacaaaagcgtgatctcatccaagatgaactaagtttggttcgttgaaatgctaacggccagttggtcaaaaagaaacttccaaaagtcggcataccgtttgtcttgtttggtattgattgacgaatgctcaaaaataatctcattaatgcttagcgcagtctctctatcgcttctgaaccccggtgcacctgtgccgaaacgcaaatggggaaacacccgctttttggatgattatgcattgtctccacattgtatgcttccaagattctggtgggaatactgctgatagcctaacgttcatgatcaaaatttaactgttctaacccctacttgacagcaatatataaacagaaggaagctgccctgtcttaaacctttttttttatcatcattattagcttactttcataattgcgactggttccaattgacaagcttttgattttaacgacttttaacgacaacttgagaagatcaaaaaacaactaattattcgaaacg

SEQ ID NO: 87 - DNA, 997 bp, synthetic construct
  source 1..997: mol_type = other DNA
ggcttggggttgagcatgtttatgaatgtttatgcttatgttctctcatcctgtatatatgcagaagcggaaaggagtagcttgaggactgatgggtagcggtgattcttcagtattaaaattatggatcttgcaatgcattcttttgttcgtataaagtctttcctaagatcataataccagcaatgaccgttgccattcactttctgcccgccactgagcctgaccgaagctatccatccgaggggccccaagccctgtagccttaacattcttccgaagtcactgcagtgttattacctgtctggaacctatgtcatattaggatagaatagagaagatgctgactgatatagctcagcactctgaagcctcaggcagtccacaggcccagggatatgaggatttgacaaggaaaggtgtgacgtactcctaccccgtcctcgcgactatttctgcagtctgagaaaagaagacgcatagccttgaacatagggcattggctgcagtgaaacgccccagttactacttatcgcagcatacgtgacgagaactcagtggttatccttccagtatagtgtagaacatctgaatacatcagaacagaagcccgatacctcccgtaactatccattccatgacctaaagatggtacaaggaaaaatttgcttgctgtttcatgatctagctcctgagacacggagattcctcccttcctatggtcaaaaaggcagcccattcccaaaggagggcttcatcgcttgatgagttctcgaatacaggctaatatatattatcatcatcatatcgacacctaccttgaccaactgtaatatagtgtttccttcagtatgtgacaacgaagcgtactcttggggcaccacagcttgtgctcgcggcgaattccagtcgcaatccgaaacttgcgggaagatagaatgacaacctacagtcacagatctattaagtgtgatttaaatgggctatattggccttg

SEQ ID NO: 88 - DNA, 2187 bp, synthetic construct
  source 1..2187: mol_type = other DNA
ctagtaaccaaaggaaaggaacagatagataaaattccgagactgtcaaattaggtttttttctttttttttggcgggagtcagtgggccgaaatatgttcttggcctagaacttaatctggtttgatcatgccaatacttgcctgagtgcccgactttttgcccacccttttgccttctgtctatccttcaaaacccacctgttttccagccgtatcttcgctcgcatctacacatactgtgccatatcttgtgtgtagccggacgtgactatgaccaaaaacaaacaaggagaactgttcgccgatttgtaacactcctgcatccatccaagtgggtatgcgctatgcaatgttaagctaggtcaggtcagaccaggtccaaggacagcaacttgactgtatgcaacctttaccatctttgcacagaacatacttgtagctagctagttacacttatggaccgaaaaggcaccccaccatgtctgtccggctttagagtacggccgcagaccgctgatttgccttgccaagcagtagtcacaatgcatcgcatgagcacacgggcacgggcacgggcacaggaaccattggcaaaaataccagatacactataccgacgtatatcaagcccaagtttaaaattcctaaatttccgcggggatcgactcataaaatagtaaccttctaatgcgtatctattgactaccaaccattagtgtggttgcagaaggcggaattctcccttcttcgaattcagcttgctttttcattttttattttccatttttcagtttttgtttgtgtcgaatttagccagttgcttctccaagatgaaaaaaacccctgcgcagtttctgtgctgcaagatcctaatcgacttttccaccccccacaaaagtaaatgttcttttgttacattcgcgtgggtagctagctccccgaatcttcaaaggacttagggactgcactacatcagagtgtgttcacctggtttgctgcctggtttgaaagaaaagagcagggaactcgcgggttcccggcgaataatcatgcgatagtcctttggccttccaagtcgcatgtagagtagacaacagacagggagggcaggaaggatctttcactgagatcctgtatcttgttgggtaagtcggatgaaaggggaatcgtatgagattggagaggatgcggaagaggtaacgccttttgttaacttgtttaattattatggggcaggcgagagggggaggaatgtatgtgtgtgaggcgggcgagacggagccatccaggccaggtagaaatagagaaagccgaatgttagacaatatggcagcgtagtagagtaggtaggtaggcaagtactgctagcaaagaggagaagggtaagctcactcttcgcattccacaccgttagtgtgtcagtttgaacaaaaaaacaatcatcataccaattgatggactgtggactggcttttggaacggcttttcggactgcgattattcgtgaggaatcaaggtaggaatttggtcatatttacggacaacagtgggtgattcccatatggagtaggaaaacgagatcatggtatcctcagatatgttgcggaattctgttcaccgcaaagttcagggtgctctggtgggtttcggttggtctttgctttgcttctcccttgtcttgcatgttaataatagcctagcctgtgagccgaaacttagggtaggcttagtgttggaacgtacatatgtatcacgttgacttggtttaaccaggcgacctggtagccagccatacccacacacgttttttgtatcttcagtatagttgtgaaaagtgtagcggaaatttgtggtccgagcaacagcgtctttttctagtagtgcggtcggttacttggttgacattggtatttggactttgttgctacaccattcactacttgaagtcgagtgtgaagggtatgatttctagtggtgaacacctttagttacgtaatgttttcattgctgttttacttgagatttcgattgagaaaaaggtatttaatagctcgaatcaatgtgttatcattgtgaagatgttcttccctaactcgaaaggtatatgaggcttgtgtttcttaggagaattattattcttttgttatgttgcgcttgtagttggaaaaggtgaagagacaaaagc

SEQ ID NO: 89 - DNA, 19 bp, synthetic construct
  source 1..19: mol_type = other DNA
taatacgactcactatagg

SEQ ID NO: 90 - DNA, 477 bp, synthetic construct
  source 1..477: mol_type = other DNA
tttttgtagaaatgtcttggtgtcctcgtccaatcaggtagccatctctgaaatatctggctccgttgcaactccgaacgacctgctggcaacgtaaaattctccggggtaaaacttaaatgtggagtaatggaaccagaaacgtctcttcccttctctctccttccaccgcccgttaccgtccctaggaaattttactctgctggagagcttcttctacggcccccttgcagcaatgctcttcccagcattacgttgcgggtaaaacggaggtcgtgtacccgacctagcagcccagggatggaaaagtcccggccgtcgctggcaataatagcgggcggacgcatgtcatgagattattggaaaccaccagaatcgaatataaaaggcgaacacctttcccaattttggtttctcctgacccaaagactttaaatttaatttatttgtccctatttcaatcaattgaacaactat

SEQ ID NO: 91 - DNA, 412 bp, synthetic construct
  source 1..412: mol_type = other DNA
atagcttcaaaatgtctctactccttttttactcttccagattttctcggactccgcgcaccgccgtaccacttcaaaacacccaagcaacagcatactaaattccccctcctccttcctctagggtgccgttaattacccgtactaaaggtttggaaaaggaaaaagagaccgcctcgtccctttttcttcgtcggagaaggcaataaaaatttttatcacgtttctttctcttgaaaacttttttttcgatttttgtctctttcgacgacctcccattgatatttgagttaacaaacggtcttcaatttctcaagtttcagcttcatttttcctgttctattacaactttttttacttcttgctcattggaaagaaagcatagcaatctaatctaagttttaattacaaa

SEQ ID NO: 92 - DNA, 703 bp, synthetic construct
  source 1..703: mol_type = other DNA
tgtacaatatggacttcctcttttctggcaaccaaacccatacatcgggattcctataataccttcgttggtctccctaacatgtaggtggcggaggggagatatacaatagaacagataccagacaagacataatgggctaaacaagactacaccaattacactgcctcattgatggtggtacataacgaactaatactgtagccctagacttgatagccatcatcatatcgaagtttcactaccctttttccatttgccatctattgaagtaataataggcgcatgcaacttcttttctttttttttcttttctctctcccccgttgttgtctcaccatatccgcaatgacaaaaaaatgatggaagacactaaaggaaaaaattaacgacaaagacagcaccaacagatgtcgttgttccagagctgatgaggggtatctcgaagcacacgaaactttttccttccttcattcacgcacactactctctaatgagcaacggtatacggccttccttccagttacttgaatttgaaataaaaaaaagtttgctgtcttgctatcaagtataaatagacctgcaattattaatcttttgtttcctcgtcattgttctcgttccctttcttccttgtttctttttctgcacaatatttcaagctataccaagcatacaatcaagcaattccagatctgccacc

SEQ ID NO: 93 - protein, 592 aa, synthetic construct
MKMMLVRRFRVLILMVFLVACALHIALDLLPRLERRGARPSGEPGCSCAQPAAEVAAPGWAQVRGRPGEPPAASSAAGDAGWPNKHTLRILQDFSSDPSSNLSSHSLEKLPPAAEPAERALRGRDPGALRPHDPAHRPLLRDPGPRRSESPPGPGGDASLLARLFEHPLYRVAVPPLTEEDVLFNVNSDTRLSPKAAENPDWPHAGAEGAEFLSPGEAAVDSYPNWLKFHIGINRYELYSRHNPAIEALLHDLSSQRITSVAMKSGGTQLKLIMTFQNYGQALFKPMKQTREQETPPDFFYFSDYERHNAEIAAFHLDRILDFRRVPPVAGRMVNMTKEIRDVTRDKKLWRTFFISPANNICFYGECSYYCSTEHALCGKPDQIEGSLAAFLPDLSLAKRKTWRNPWRRSYHKRKKAEWEVDPDYCEEVKQTPPYDSSHRILDVMDMTIFDFLMGNMDRHHYETFEKFGNETFIIHLDNGRGFGKYSHDELSILVPLQQCCRIRKSTYLRLQLLAKEEYKLSLLMAESLRGDQVAPVLYQPHLEALDRRLRVVLKAVRDCVERNGLHSVVDDDLDTEHRAASARDYKDDDDK

SEQ ID NO: 94 - protein, 562 aa, synthetic construct
MRLERRGARPSGEPGCSCAQPAAEVAAPGWAQVRGRPGEPPAASSAAGDAGWPNKHTLRILQDFSSDPSSNLSSHSLEKLPPAAEPAERALRGRDPGALRPHDPAHRPLLRDPGPRRSESPPGPGGDASLLARLFEHPLYRVAVPPLTEEDVLFNVNSDTRLSPKAAENPDWPHAGAEGAEFLSPGEAAVDSYPNWLKFHIGINRYELYSRHNPAIEALLHDLSSQRITSVAMKSGGTQLKLIMTFQNYGQALFKPMKQTREQETPPDFFYFSDYERHNAEIAAFHLDRILDFRRVPPVAGRMVNMTKEIRDVTRDKKLWRTFFISPANNICFYGECSYYCSTEHALCGKPDQIEGSLAAFLPDLSLAKRKTWRNPWRRSYHKRKKAEWEVDPDYCEEVKQTPPYDSSHRILDVMDMTIFDFLMGNMDRHHYETFEKFGNETFIIHLDNGRGFGKYSHDELSILVPLQQCCRIRKSTYLRLQLLAKEEYKLSLLMAESLRGDQVAPVLYQPHLEALDRRLRVVLKAVRDCVERNGLHSVVDDDLDTEHRAASARDYKDDDDK

SEQ ID NO: 95 - protein, 619 aa, synthetic construct
MALFLSKRLLRFTVIAGAVIVLLLTLNSNSRTQQYIPSSISAAFDFTSGSISPEQQVIRLERRGARPSGEPGCSCAQPAAEVAAPGWAQVRGRPGEPPAASSAAGDAGWPNKHTLRILQDFSSDPSSNLSSHSLEKLPPAAEPAERALRGRDPGALRPHDPAHRPLLRDPGPRRSESPPGPGGDASLLARLFEHPLYRVAVPPLTEEDVLFNVNSDTRLSPKAAENPDWPHAGAEGAEFLSPGEAAVDSYPNWLKFHIGINRYELYSRHNPAIEALLHDLSSQRITSVAMKSGGTQLKLIMTFQNYGQALFKPMKQTREQETPPDFFYFSDYERHNAEIAAFHLDRILDFRRVPPVAGRMVNMTKEIRDVTRDKKLWRTFFISPANNICFYGECSYYCSTEHALCGKPDQIEGSLAAFLPDLSLAKRKTWRNPWRRSYHKRKKAEWEVDPDYCEEVKQTPPYDSSHRILDVMDMTIFDFLMGNMDRHHYETFEKFGNETFIIHLDNGRGFGKYSHDELSILVPLQQCCRIRKSTYLRLQLLAKEEYKLSLLMAESLRGDQVAPVLYQPHLEALDRRLRVVLKAVRDCVERNGLHSVVDDDLDTEHRAASARDYKDDDDK

SEQ ID NO: 96 - protein, 641 aa, synthetic construct
MALFLSKRLLRFTVIAGAVIVLLLTLNSNSRTQQYIPSSISAAFDFTSGSISPEQQVISEENDAKKLEQSALNSEASEDSRLERRGARPSGEPGCSCAQPAAEVAAPGWAQVRGRPGEPPAASSAAGDAGWPNKHTLRILQDFSSDPSSNLSSHSLEKLPPAAEPAERALRGRDPGALRPHDPAHRPLLRDPGPRRSESPPGPGGDASLLARLFEHPLYRVAVPPLTEEDVLFNVNSDTRLSPKAAENPDWPHAGAEGAEFLSPGEAAVDSYPNWLKFHIGINRYELYSRHNPAIEALLHDLSSQRITSVAMKSGGTQLKLIMTFQNYGQALFKPMKQTREQETPPDFFYFSDYERHNAEIAAFHLDRILDFRRVPPVAGRMVNMTKEIRDVTRDKKLWRTFFISPANNICFYGECSYYCSTEHALCGKPDQIEGSLAAFLPDLSLAKRKTWRNPWRRSYHKRKKAEWEVDPDYCEEVKQTPPYDSSHRILDVMDMTIFDFLMGNMDRHHYETFEKFGNETFIIHLDNGRGFGKYSHDELSILVPLQQCCRIRKSTYLRLQLLAKEEYKLSLLMAESLRGDQVAPVLYQPHLEALDRRLRVVLKAVRDCVERNGLHSVVDDDLDTEHRAASARDYKDDDDK

SEQ ID NO: 97 - protein, 663 aa, synthetic construct
MALFLSKRLLRFTVIAGAVIVLLLTLNSNSRTQQYIPSSISAAFDFTSGSISPEQQVISEENDAKKLEQSALNSEASEDSEAMDEESKALKAAAEKADAPIDRLERRGARPSGEPGCSCAQPAAEVAAPGWAQVRGRPGEPPAASSAAGDAGWPNKHTLRILQDFSSDPSSNLSSHSLEKLPPAAEPAERALRGRDPGALRPHDPAHRPLLRDPGPRRSESPPGPGGDASLLARLFEHPLYRVAVPPLTEEDVLFNVNSDTRLSPKAAENPDWPHAGAEGAEFLSPGEAAVDSYPNWLKFHIGINRYELYSRHNPAIEALLHDLSSQRITSVAMKSGGTQLKLIMTFQNYGQALFKPMKQTREQETPPDFFYFSDYERHNAEIAAFHLDRILDFRRVPPVAGRMVNMTKEIRDVTRDKKLWRTFFISPANNICFYGECSYYCSTEHALCGKPDQIEGSLAAFLPDLSLAKRKTWRNPWRRSYHKRKKAEWEVDPDYCEEVKQTPPYDSSHRILDVMDMTIFDFLMGNMDRHHYETFEKFGNETFIIHLDNGRGFGKYSHDELSILVPLQQCCRIRKSTYLRLQLLAKEEYKLSLLMAESLRGDQVAPVLYQPHLEALDRRLRVVLKAVRDCVERNGLHSVVDDDLDTEHRAASARDYKDDDDK

SEQ ID NO: 98 - protein, 592 aa, synthetic construct
MVHIGFRSLKAVFILALSSLILYGIVTTFDGRLERRGARPSGEPGCSCAQPAAEVAAPGWAQVRGRPGEPPAASSAAGDAGWPNKHTLRILQDFSSDPSSNLSSHSLEKLPPAAEPAERALRGRDPGALRPHDPAHRPLLRDPGPRRSESPPGPGGDASLLARLFEHPLYRVAVPPLTEEDVLFNVNSDTRLSPKAAENPDWPHAGAEGAEFLSPGEAAVDSYPNWLKFHIGINRYELYSRHNPAIEALLHDLSSQRITSVAMKSGGTQLKLIMTFQNYGQALFKPMKQTREQETPPDFFYFSDYERHNAEIAAFHLDRILDFRRVPPVAGRMVNMTKEIRDVTRDKKLWRTFFISPANNICFYGECSYYCSTEHALCGKPDQIEGSLAAFLPDLSLAKRKTWRNPWRRSYHKRKKAEWEVDPDYCEEVKQTPPYDSSHRILDVMDMTIFDFLMGNMDRHHYETFEKFGNETFIIHLDNGRGFGKYSHDELSILVPLQQCCRIRKSTYLRLQLLAKEEYKLSLLMAESLRGDQVAPVLYQPHLEALDRRLRVVLKAVRDCVERNGLHSVVDDDLDTEHRAASARDYKDDDDK

SEQ ID NO: 99 - protein, 645 aa, synthetic construct
MVHIGFRSLKAVFILALSSLILYGIVTTFDGSRASRYQPPYVNHSQDPLYHSGNSYNRENATFVTLCRNEDLYSIIQSIKKVEDRLERRGARPSGEPGCSCAQPAAEVAAPGWAQVRGRPGEPPAASSAAGDAGWPNKHTLRILQDFSSDPSSNLSSHSLEKLPPAAEPAERALRGRDPGALRPHDPAHRPLLRDPGPRRSESPPGPGGDASLLARLFEHPLYRVAVPPLTEEDVLFNVNSDTRLSPKAAENPDWPHAGAEGAEFLSPGEAAVDSYPNWLKFHIGINRYELYSRHNPAIEALLHDLSSQRITSVAMKSGGTQLKLIMTFQNYGQALFKPMKQTREQETPPDFFYFSDYERHNAEIAAFHLDRILDFRRVPPVAGRMVNMTKEIRDVTRDKKLWRTFFISPANNICFYGECSYYCSTEHALCGKPDQIEGSLAAFLPDLSLAKRKTWRNPWRRSYHKRKKAEWEVDPDYCEEVKQTPPYDSSHRILDVMDMTIFDFLMGNMDRHHYETFEKFGNETFIIHLDNGRGFGKYSHDELSILVPLQQCCRIRKSTYLRLQLLAKEEYKLSLLMAESLRGDQVAPVLYQPHLEALDRRLRVVLKAVRDCVERNGLHSVVDDDLDTEHRAASARDYKDDDDK

SEQ ID NO: 100 - protein, 711 aa, synthetic construct
MVHIGFRSLKAVFILALSSLILYGIVTTFDGSRASRYQPPYVNHSQDPLYHSGNSYNRENATFVTLCRNEDLYSIIQSIKKVEDRFNNKFAYDWVFLNEVPFTDEFKERTSVLISGQAKYGLIPKEHWSYPDYIDQERAAESRRQLEDQHRLERRGARPSGEPGCSCAQPAAEVAAPGWAQVRGRPGEPPAASSAAGDAGWPNKHTLRILQDFSSDPSSNLSSHSLEKLPPAAEPAERALRGRDPGALRPHDPAHRPLLRDPGPRRSESPPGPGGDASLLARLFEHPLYRVAVPPLTEEDVLFNVNSDTRLSPKAAENPDWPHAGAEGAEFLSPGEAAVDSYPNWLKFHIGINRYELYSRHNPAIEALLHDLSSQRITSVAMKSGGTQLKLIMTFQNYGQALFKPMKQTREQETPPDFFYFSDYERHNAEIAAFHLDRILDFRRVPPVAGRMVNMTKEIRDVTRDKKLWRTFFISPANNICFYGECSYYCSTEHALCGKPDQIEGSLAAFLPDLSLAKRKTWRNPWRRSYHKRKKAEWEVDPDYCEEVKQTPPYDSSHRILDVMDMTIFDFLMGNMDRHHYETFEKFGNETFIIHLDNGRGFGKYSHDELSILVPLQQCCRIRKSTYLRLQLLAKEEYKLSLLMAESLRGDQVAPVLYQPHLEALDRRLRVVLKAVRDCVERNGLHSVVDDDLDTEHRAASARDYKDDDDK

SEQ ID NO: 101 - protein, 597 aa, synthetic construct
MLLTKRFSKLFKLTFIVLILCGLFVITNKYMDENTSRLERRGARPSGEPGCSCAQPAAEVAAPGWAQVRGRPGEPPAASSAAGDAGWPNKHTLRILQDFSSDPSSNLSSHSLEKLPPAAEPAERALRGRDPGALRPHDPAHRPLLRDPGPRRSESPPGPGGDASLLARLFEHPLYRVAVPPLTEEDVLFNVNSDTRLSPKAAENPDWPHAGAEGAEFLSPGEAAVDSYPNWLKFHIGINRYELYSRHNPAIEALLHDLSSQRITSVAMKSGGTQLKLIMTFQNYGQALFKPMKQTREQETPPDFFYFSDYERHNAEIAAFHLDRILDFRRVPPVAGRMVNMTKEIRDVTRDKKLWRTFFISPANNICFYGECSYYCSTEHALCGKPDQIEGSLAAFLPDLSLAKRKTWRNPWRRSYHKRKKAEWEVDPDYCEEVKQTPPYDSSHRILDVMDMTIFDFLMGNMDRHHYETFEKFGNETFIIHLDNGRGFGKYSHDELSILVPLQQCCRIRKSTYLRLQLLAKEEYKLSLLMAESLRGDQVAPVLYQPHLEALDRRLRVVLKAVRDCVERNGLHSVVDDDLDTEHRAASARDYKDDDDK

SEQ ID NO: 102 - protein, 658 aa, synthetic construct
MLLTKRFSKLFKLTFIVLILCGLFVITNKYMDENTSVKEYKEYLDRYVQSYSNKYSSSSDAASADDSTPLRDNDEAGNEKLKSFYNNVFNFLMVDSPRLERRGARPSGEPGCSCAQPAAEVAAPGWAQVRGRPGEPPAASSAAGDAGWPNKHTLRILQDFSSDPSSNLSSHSLEKLPPAAEPAERALRGRDPGALRPHDPAHRPLLRDPGPRRSESPPGPGGDASLLARLFEHPLYRVAVPPLTEEDVLFNVNSDTRLSPKAAENPDWPHAGAEGAEFLSPGEAAVDSYPNWLKFHIGINRYELYSRHNPAIEALLHDLSSQRITSVAMKSGGTQLKLIMTFQNYGQALFKPMKQTREQETPPDFFYFSDYERHNAEIAAFHLDRILDFRRVPPVAGRMVNMTKEIRDVTRDKKLWRTFFISPANNICFYGECSYYCSTEHALCGKPDQIEGSLAAFLPDLSLAKRKTWRNPWRRSYHKRKKAEWEVDPDYCEEVKQTPPYDSSHRILDVMDMTIFDFLMGNMDRHHYETFEKFGNETFIIHLDNGRGFGKYSHDELSILVPLQQCCRIRKSTYLRLQLLAKEEYKLSLLMAESLRGDQVAPVLYQPHLEALDRRLRVVLKAVRDCVERNGLHSVVDDDLDTEHRAASARDYKDDDDK

SEQ ID NO: 103 - protein, 711 aa, synthetic construct
MLLTKRFSKLFKLTFIVLILCGLFVITNKYMDENTSVKEYKEYLDRYVQSYSNKYSSSSDAASADDSTPLRDNDEAGNEKLKSFYNNVFNFLMVDSPKGSTAKQYNEACLLKGDIGDRPDHYKDLYKLSAKELSKCLELSPDEVASLTKSRLERRGARPSGEPGCSCAQPAAEVAAPGWAQVRGRPGEPPAASSAAGDAGWPNKHTLRILQDFSSDPSSNLSSHSLEKLPPAAEPAERALRGRDPGALRPHDPAHRPLLRDPGPRRSESPPGPGGDASLLARLFEHPLYRVAVPPLTEEDVLFNVNSDTRLSPKAAENPDWPHAGAEGAEFLSPGEAAVDSYPNWLKFHIGINRYELYSRHNPAIEALLHDLSSQRITSVAMKSGGTQLKLIMTFQNYGQALFKPMKQTREQETPPDFFYFSDYERHNAEIAAFHLDRILDFRRVPPVAGRMVNMTKEIRDVTRDKKLWRTFFISPANNICFYGECSYYCSTEHALCGKPDQIEGSLAAFLPDLSLAKRKTWRNPWRRSYHKRKKAEWEVDPDYCEEVKQTPPYDSSHRILDVMDMTIFDFLMGNMDRHHYETFEKFGNETFIIHLDNGRGFGKYSHDELSILVPLQQCCRIRKSTYLRLQLLAKEEYKLSLLMAESLRGDQVAPVLYQPHLEALDRRLRVVLKAVRDCVERNGLHSVVDDDLDTEHRAASARDYKDDDDK

SEQ ID NO: 104 - protein, 603 aa, synthetic construct
MLALRRFILNQRSLRSCTIPILVGALIIILVLFQLVTHRNDARLERRGARPSGEPGCSCAQPAAEVAAPGWAQVRGRPGEPPAASSAAGDAGWPNKHTLRILQDFSSDPSSNLSSHSLEKLPPAAEPAERALRGRDPGALRPHDPAHRPLLRDPGPRRSESPPGPGGDASLLARLFEHPLYRVAVPPLTEEDVLFNVNSDTRLSPKAAENPDWPHAGAEGAEFLSPGEAAVDSYPNWLKFHIGINRYELYSRHNPAIEALLHDLSSQRITSVAMKSGGTQLKLIMTFQNYGQALFKPMKQTREQETPPDFFYFSDYERHNAEIAAFHLDRILDFRRVPPVAGRMVNMTKEIRDVTRDKKLWRTFFISPANNICFYGECSYYCSTEHALCGKPDQIEGSLAAFLPDLSLAKRKTWRNPWRRSYHKRKKAEWEVDPDYCEEVKQTPPYDSSHRILDVMDMTIFDFLMGNMDRHHYETFEKFGNETFIIHLDNGRGFGKYSHDELSILVPLQQCCRIRKSTYLRLQLLAKEEYKLSLLMAESLRGDQVAPVLYQPHLEALDRRLRVVLKAVRDCVERNGLHSVVDDDLDTEHRAASARDYKDDDDK

SEQ ID NO: 105 - protein, 654 aa, synthetic construct
MLALRRFILNQRSLRSCTIPILVGALIIILVLFQLVTHRNDALIRSSNVNSTNKKTLKDADPKVLIEAFGSPEVDPVDTIPVSPLELVPFYDQRLERRGARPSGEPGCSCAQPAAEVAAPGWAQVRGRPGEPPAASSAAGDAGWPNKHTLRILQDFSSDPSSNLSSHSLEKLPPAAEPAERALRGRDPGALRPHDPAHRPLLRDPGPRRSESPPGPGGDASLLARLFEHPLYRVAVPPLTEEDVLFNVNSDTRLSPKAAENPDWPHAGAEGAEFLSPGEAAVDSYPNWLKFHIGINRYELYSRHNPAIEALLHDLSSQRITSVAMKSGGTQLKLIMTFQNYGQALFKPMKQTREQETPPDFFYFSDYERHNAEIAAFHLDRILDFRRVPPVAGRMVNMTKEIRDVTRDKKLWRTFFISPANNICFYGECSYYCSTEHALCGKPDQIEGSLAAFLPDLSLAKRKTWRNPWRRSYHKRKKAEWEVDPDYCEEVKQTPPYDSSHRILDVMDMTIFDFLMGNMDRHHYETFEKFGNETFIIHLDNGRGFGKYSHDELSILVPLQQCCRIRKSTYLRLQLLAKEEYKLSLLMAESLRGDQVAPVLYQPHLEALDRRLRVVLKAVRDCVERNGLHSVVDDDLDTEHRAASARDYKDDDDK

SEQ ID NO: 106 - protein, 714 aa, synthetic construct
MLALRRFILNQRSLRSCTIPILVGALIIILVLFQLVTHRNDALIRSSNVNSTNKKTLKDADPKVLIEAFGSPEVDPVDTIPVSPLELVPFYDQSIDTKRSSSWLINKKGYYKHFNELSLTDRCKFYFRTLYTLDDEWTNSVKKLEYSINDNEGRLERRGARPSGEPGCSCAQPAAEVAAPGWAQVRGRPGEPPAASSAAGDAGWPNKHTLRILQDFSSDPSSNLSSHSLEKLPPAAEPAERALRGRDPGALRPHDPAHRPLLRDPGPRRSESPPGPGGDASLLARLFEHPLYRVAVPPLTEEDVLFNVNSDTRLSPKAAENPDWPHAGAEGAEFLSPGEAAVDSYPNWLKFHIGINRYELYSRHNPAIEALLHDLSSQRITSVAMKSGGTQLKLIMTFQNYGQALFKPMKQTREQETPPDFFYFSDYERHNAEIAAFHLDRILDFRRVPPVAGRMVNMTKEIRDVTRDKKLWRTFFISPANNICFYGECSYYCSTEHALCGKPDQIEGSLAAFLPDLSLAKRKTWRNPWRRSYHKRKKAEWEVDPDYCEEVKQTPPYDSSHRILDVMDMTIFDFLMGNMDRHHYETFEKFGNETFIIHLDNGRGFGKYSHDELSILVPLQQCCRIRKSTYLRLQLLAKEEYKLSLLMAESLRGDQVAPVLYQPHLEALDRRLRVVLKAVRDCVERNGLHSVVDDDLDTEHRAASARDYKDDDDK

SEQ ID NO: 107 - protein, 591 aa, synthetic construct
MHVLLSKKIARFLLISFVFVLALMVTINHPRLERRGARPSGEPGCSCAQPAAEVAAPGWAQVRGRPGEPPAASSAAGDAGWPNKHTLRILQDFSSDPSSNLSSHSLEKLPPAAEPAERALRGRDPGALRPHDPAHRPLLRDPGPRRSESPPGPGGDASLLARLFEHPLYRVAVPPLTEEDVLFNVNSDTRLSPKAAENPDWPHAGAEGAEFLSPGEAAVDSYPNWLKFHIGINRYELYSRHNPAIEALLHDLSSQRITSVAMKSGGTQLKLIMTFQNYGQALFKPMKQTREQETPPDFFYFSDYERHNAEIAAFHLDRILDFRRVPPVAGRMVNMTKEIRDVTRDKKLWRTFFISPANNICFYGECSYYCSTEHALCGKPDQIEGSLAAFLPDLSLAKRKTWRNPWRRSYHKRKKAEWEVDPDYCEEVKQTPPYDSSHRILDVMDMTIFDFLMGNMDRHHYETFEKFGNETFIIHLDNGRGFGKYSHDELSILVPLQQCCRIRKSTYLRLQLLAKEEYKLSLLMAESLRGDQVAPVLYQPHLEALDRRLRVVLKAVRDCVERNGLHSVVDDDLDTEHRAASARDYKDDDDK

SEQ ID NO: 108 - protein, 646 aa, synthetic construct
MHVLLSKKIARFLLISFVFVLALMVTINHPKTKQMSEQYVTPYLPKSLQPIAKISAEEQRRIQSEQEEAELKQSLEGEAIRNATVRLERRGARPSGEPGCSCAQPAAEVAAPGWAQVRGRPGEPPAASSAAGDAGWPNKHTLRILQDFSSDPSSNLSSHSLEKLPPAAEPAERALRGRDPGALRPHDPAHRPLLRDPGPRRSESPPGPGGDASLLARLFEHPLYRVAVPPLTEEDVLFNVNSDTRLSPKAAENPDWPHAGAEGAEFLSPGEAAVDSYPNWLKFHIGINRYELYSRHNPAIEALLHDLSSQRITSVAMKSGGTQLKLIMTFQNYGQALFKPMKQTREQETPPDFFYFSDYERHNAEIAAFHLDRILDFRRVPPVAGRMVNMTKEIRDVTRDKKLWRTFFISPANNICFYGECSYYCSTEHALCGKPDQIEGSLAAFLPDLSLAKRKTWRNPWRRSYHKRKKAEWEVDPDYCEEVKQTPPYDSSHRILDVMDMTIFDFLMGNMDRHHYETFEKFGNETFIIHLDNGRGFGKYSHDELSILVPLQQCCRIRKSTYLRLQLLAKEEYKLSLLMAESLRGDQVAPVLYQPHLEALDRRLRVVLKAVRDCVERNGLHSVVDDDLDTEHRAASARDYKDDDDK

SEQ ID NO: 109 - protein, 721 aa, synthetic construct
MHVLLSKKIARFLLISFVFVLALMVTINHPKTKQMSEQYVTPYLPKSLQPIAKISAEEQRRIQSEQEEAELKQSLEGEAIRNATVNAIKEKIKSYGGNETTLGFMVPSYINHRGSPPKACFVSLITERDSMTQILQSIDEVQVKFNKNFAYPWVFISQGERLERRGARPSGEPGCSCAQPAAEVAAPGWAQVRGRPGEPPAASSAAGDAGWPNKHTLRILQDFSSDPSSNLSSHSLEKLPPAAEPAERALRGRDPGALRPHDPAHRPLLRDPGPRRSESPPGPGGDASLLARLFEHPLYRVAVPPLTEEDVLFNVNSDTRLSPKAAENPDWPHAGAEGAEFLSPGEAAVDSYPNWLKFHIGINRYELYSRHNPAIEALLHDLSSQRITSVAMKSGGTQLKLIMTFQNYGQALFKPMKQTREQETPPDFFYFSDYERHNAEIAAFHLDRILDFRRVPPVAGRMVNMTKEIRDVTRDKKLWRTFFISPANNICFYGECSYYCSTEHALCGKPDQIEGSLAAFLPDLSLAKRKTWRNPWRRSYHKRKKAEWEVDPDYCEEVKQTPPYDSSHRILDVMDMTIFDFLMGNMDRHHYETFEKFGNETFIIHLDNGRGFGKYSHDELSILVPLQQCCRIRKSTYLRLQLLAKEEYKLSLLMAESLRGDQVAPVLYQPHLEALDRRLRVVLKAVRDCVERNGLHSVVDDDLDTEHRAASARDYKDDDDK

SEQ ID NO: 110 - protein, 530 aa, synthetic construct
MRGRPGEPPAASSAAGDAGWPNKHTLRILQDFSSDPSSNLSSHSLEKLPPAAEPAERALRGRDPGALRPHDPAHRPLLRDPGPRRSESPPGPGGDASLLARLFEHPLYRVAVPPLTEEDVLFNVNSDTRLSPKAAENPDWPHAGAEGAEFLSPGEAAVDSYPNWLKFHIGINRYELYSRHNPAIEALLHDLSSQRITSVAMKSGGTQLKLIMTFQNYGQALFKPMKQTREQETPPDFFYFSDYERHNAEIAAFHLDRILDFRRVPPVAGRMVNMTKEIRDVTRDKKLWRTFFISPANNICFYGECSYYCSTEHALCGKPDQIEGSLAAFLPDLSLAKRKTWRNPWRRSYHKRKKAEWEVDPDYCEEVKQTPPYDSSHRILDVMDMTIFDFLMGNMDRHHYETFEKFGNETFIIHLDNGRGFGKYSHDELSILVPLQQCCRIRKSTYLRLQLLAKEEYKLSLLMAESLRGDQVAPVLYQPHLEALDRRLRVVLKAVRDCVERNGLHSVVDDDLDTEHRAASARDYKDDDDK

SEQ ID NO: 111 - protein, 587 aa, synthetic construct
MALFLSKRLLRFTVIAGAVIVLLLTLNSNSRTQQYIPSSISAAFDFTSGSISPEQQVIRGRPGEPPAASSAAGDAGWPNKHTLRILQDFSSDPSSNLSSHSLEKLPPAAEPAERALRGRDPGALRPHDPAHRPLLRDPGPRRSESPPGPGGDASLLARLFEHPLYRVAVPPLTEEDVLFNVNSDTRLSPKAAENPDWPHAGAEGAEFLSPGEAAVDSYPNWLKFHIGINRYELYSRHNPAIEALLHDLSSQRITSVAMKSGGTQLKLIMTFQNYGQALFKPMKQTREQETPPDFFYFSDYERHNAEIAAFHLDRILDFRRVPPVAGRMVNMTKEIRDVTRDKKLWRTFFISPANNICFYGECSYYCSTEHALCGKPDQIEGSLAAFLPDLSLAKRKTWRNPWRRSYHKRKKAEWEVDPDYCEEVKQTPPYDSSHRILDVMDMTIFDFLMGNMDRHHYETFEKFGNETFIIHLDNGRGFGKYSHDELSILVPLQQCCRIRKSTYLRLQLLAKEEYKLSLLMAESLRGDQVAPVLYQPHLEALDRRLRVVLKAVRDCVERNGLHSVVDDDLDTEHRAASARDYKDDDDK

SEQ ID NO: 112 - protein, 609 aa, synthetic construct
MALFLSKRLLRFTVIAGAVIVLLLTLNSNSRTQQYIPSSISAAFDFTSGSISPEQQVISEENDAKKLEQSALNSEASEDSRGRPGEPPAASSAAGDAGWPNKHTLRILQDFSSDPSSNLSSHSLEKLPPAAEPAERALRGRDPGALRPHDPAHRPLLRDPGPRRSESPPGPGGDASLLARLFEHPLYRVAVPPLTEEDVLFNVNSDTRLSPKAAENPDWPHAGAEGAEFLSPGEAAVDSYPNWLKFHIGINRYELYSRHNPAIEALLHDLSSQRITSVAMKSGGTQLKLIMTFQNYGQALFKPMKQTREQETPPDFFYFSDYERHNAEIAAFHLDRILDFRRVPPVAGRMVNMTKEIRDVTRDKKLWRTFFISPANNICFYGECSYYCSTEHALCGKPDQIEGSLAAFLPDLSLAKRKTWRNPWRRSYHKRKKAEWEVDPDYCEEVKQTPPYDSSHRILDVMDMTIFDFLMGNMDRHHYETFEKFGNETFIIHLDNGRGFGKYSHDELSILVPLQQCCRIRKSTYLRLQLLAKEEYKLSLLMAESLRGDQVAPVLYQPHLEALDRRLRVVLKAVRDCVERNGLHSVVDDDLDTEHRAASARDYKDDDDK

SEQ ID NO: 113 - protein, 631 aa, synthetic construct
MALFLSKRLLRFTVIAGAVIVLLLTLNSNSRTQQYIPSSISAAFDFTSGSISPEQQVISEENDAKKLEQSALNSEASEDSEAMDEESKALKAAAEKADAPIDRGRPGEPPAASSAAGDAGWPNKHTLRILQDFSSDPSSNLSSHSLEKLPPAAEPAERALRGRDPGALRPHDPAHRPLLRDPGPRRSESPPGPGGDASLLARLFEHPLYRVAVPPLTEEDVLFNVNSDTRLSPKAAENPDWPHAGAEGAEFLSPGEAAVDSYPNWLKFHIGINRYELYSRHNPAIEALLHDLSSQRITSVAMKSGGTQLKLIMTFQNYGQALFKPMKQTREQETPPDFFYFSDYERHNAEIAAFHLDRILDFRRVPPVAGRMVNMTKEIRDVTRDKKLWRTFFISPANNICFYGECSYYCSTEHALCGKPDQIEGSLAAFLPDLSLAKRKTWRNPWRRSYHKRKKAEWEVDPDYCEEVKQTPPYDSSHRILDVMDMTIFDFLMGNMDRHHYETFEKFGNETFIIHLDNGRGFGKYSHDELSILVPLQQCCRIRKSTYLRLQLLAKEEYKLSLLMAESLRGDQVAPVLYQPHLEALDRRLRVVLKAVRDCVERNGLHSVVDDDLDTEHRAASARDYKDDDDK

SEQ ID NO: 114 - protein, 560 aa, synthetic construct
MVHIGFRSLKAVFILALSSLILYGIVTTFDGRGRPGEPPAASSAAGDAGWPNKHTLRILQDFSSDPSSNLSSHSLEKLPPAAEPAERALRGRDPGALRPHDPAHRPLLRDPGPRRSESPPGPGGDASLLARLFEHPLYRVAVPPLTEEDVLFNVNSDTRLSPKAAENPDWPHAGAEGAEFLSPGEAAVDSYPNWLKFHIGINRYELYSRHNPAIEALLHDLSSQRITSVAMKSGGTQLKLIMTFQNYGQALFKPMKQTREQETPPDFFYFSDYERHNAEIAAFHLDRILDFRRVPPVAGRMVNMTKEIRDVTRDKKLWRTFFISPANNICFYGECSYYCSTEHALCGKPDQIEGSLAAFLPDLSLAKRKTWRNPWRRSYHKRKKAEWEVDPDYCEEVKQTPPYDSSHRILDVMDMTIFDFLMGNMDRHHYETFEKFGNETFIIHLDNGRGFGKYSHDELSILVPLQQCCRIRKSTYLRLQLLAKEEYKLSLLMAESLRGDQVAPVLYQPHLEALDRRLRVVLKAVRDCVERNGLHSVVDDDLDTEHRAASARDYKDDDDK

SEQ ID NO: 115 - protein, 613 aa, synthetic construct
MVHIGFRSLKAVFILALSSLILYGIVTTFDGSRASRYQPPYVNHSQDPLYHSGNSYNRENATFVTLCRNEDLYSIIQSIKKVEDRGRPGEPPAASSAAGDAGWPNKHTLRILQDFSSDPSSNLSSHSLEKLPPAAEPAERALRGRDPGALRPHDPAHRPLLRDPGPRRSESPPGPGGDASLLARLFEHPLYRVAVPPLTEEDVLFNVNSDTRLSPKAAENPDWPHAGAEGAEFLSPGEAAVDSYPNWLKFHIGINRYELYSRHNPAIEALLHDLSSQRITSVAMKSGGTQLKLIMTFQNYGQALFKPMKQTREQETPPDFFYFSDYERHNAEIAAFHLDRILDFRRVPPVAGRMVNMTKEIRDVTRDKKLWRTFFISPANNICFYGECSYYCSTEHALCGKPDQIEGSLAAFLPDLSLAKRKTWRNPWRRSYHKRKKAEWEVDPDYCEEVKQTPPYDSSHRILDVMDMTIFDFLMGNMDRHHYETFEKFGNETFIIHLDNGRGFGKYSHDELSILVPLQQCCRIRKSTYLRLQLLAKEEYKLSLLMAESLRGDQVAPVLYQPHLEALDRRLRVVLKAVRDCVERNGLHSVVDDDLDTEHRAASARDYKDDDDK

SEQ ID NO: 116 - protein, 679 aa, synthetic construct
MVHIGFRSLKAVFILALSSLILYGIVTTFDGSRASRYQPPYVNHSQDPLYHSGNSYNRENATFVTLCRNEDLYSIIQSIKKVEDRFNNKFAYDWVFLNEVPFTDEFKERTSVLISGQAKYGLIPKEHWSYPDYIDQERAAESRRQLEDQHRGRPGEPPAASSAAGDAGWPNKHTLRILQDFSSDPSSNLSSHSLEKLPPAAEPAERALRGRDPGALRPHDPAHRPLLRDPGPRRSESPPGPGGDASLLARLFEHPLYRVAVPPLTEEDVLFNVNSDTRLSPKAAENPDWPHAGAEGAEFLSPGEAAVDSYPNWLKFHIGINRYELYSRHNPAIEALLHDLSSQRITSVAMKSGGTQLKLIMTFQNYGQALFKPMKQTREQETPPDFFYFSDYERHNAEIAAFHLDRILDFRRVPPVAGRMVNMTKEIRDVTRDKKLWRTFFISPANNICFYGECSYYCSTEHALCGKPDQIEGSLAAFLPDLSLAKRKTWRNPWRRSYHKRKKAEWEVDPDYCEEVKQTPPYDSSHRILDVMDMTIFDFLMGNMDRHHYETFEKFGNETFIIHLDNGRGFGKYSHDELSILVPLQQCCRIRKSTYLRLQLLAKEEYKLSLLMAESLRGDQVAPVLYQPHLEALDRRLRVVLKAVRDCVERNGLHSVVDDDLDTEHRAASARDYKDDDDK

SEQ ID NO: 117 - protein, 565 aa, synthetic construct
MLLTKRFSKLFKLTFIVLILCGLFVITNKYMDENTSRGRPGEPPAASSAAGDAGWPNKHTLRILQDFSSDPSSNLSSHSLEKLPPAAEPAERALRGRDPGALRPHDPAHRPLLRDPGPRRSESPPGPGGDASLLARLFEHPLYRVAVPPLTEEDVLFNVNSDTRLSPKAAENPDWPHAGAEGAEFLSPGEAAVDSYPNWLKFHIGINRYELYSRHNPAIEALLHDLSSQRITSVAMKSGGTQLKLIMTFQNYGQALFKPMKQTREQETPPDFFYFSDYERHNAEIAAFHLDRILDFRRVPPVAGRMVNMTKEIRDVTRDKKLWRTFFISPANNICFYGECSYYCSTEHALCGKPDQIEGSLAAFLPDLSLAKRKTWRNPWRRSYHKRKKAEWEVDPDYCEEVKQTPPYDSSHRILDVMDMTIFDFLMGNMDRHHYETFEKFGNETFIIHLDNGRGFGKYSHDELSILVPLQQCCRIRKSTYLRLQLLAKEEYKLSLLMAESLRGDQVAPVLYQPHLEALDRRLRVVLKAVRDCVERNGLHSVVDDDLDTEHRAASARDYKDDDDK

SEQ ID NO: 118 - protein, 626 aa, synthetic construct
MLLTKRFSKLFKLTFIVLILCGLFVITNKYMDENTSVKEYKEYLDRYVQSYSNKYSSSSDAASADDSTPLRDNDEAGNEKLKSFYNNVFNFLMVDSPRGRPGEPPAASSAAGDAGWPNKHTLRILQDFSSDPSSNLSSHSLEKLPPAAEPAERALRGRDPGALRPHDPAHRPLLRDPGPRRSESPPGPGGDASLLARLFEHPLYRVAVPPLTEEDVLFNVNSDTRLSPKAAENPDWPHAGAEGAEFLSPGEAAVDSYPNWLKFHIGINRYELYSRHNPAIEALLHDLSSQRITSVAMKSGGTQLKLIMTFQNYGQALFKPMKQTREQETPPDFFYFSDYERHNAEIAAFHLDRILDFRRVPPVAGRMVNMTKEIRDVTRDKKLWRTFFISPANNICFYGECSYYCSTEHALCGKPDQIEGSLAAFLPDLSLAKRKTWRNPWRRSYHKRKKAEWEVDPDYCEEVKQTPPYDSSHRILDVMDMTIFDFLMGNMDRHHYETFEKFGNETFIIHLDNGRGFGKYSHDELSILVPLQQCCRIRKSTYLRLQLLAKEEYKLSLLMAESLRGDQVAPVLYQPHLEALDRRLRVVLKAVRDCVERNGLHSVVDDDLDTEHRAASARDYKDDDDK

SEQ ID NO: 119 - protein, 679 aa, synthetic construct
MLLTKRFSKLFKLTFIVLILCGLFVITNKYMDENTSVKEYKEYLDRYVQSYSNKYSSSSDAASADDSTPLRDNDEAGNEKLKSFYNNVFNFLMVDSPKGSTAKQYNEACLLKGDIGDRPDHYKDLYKLSAKELSKCLELSPDEVASLTKSRGRPGEPPAASSAAGDAGWPNKHTLRILQDFSSDPSSNLSSHSLEKLPPAAEPAERALRGRDPGALRPHDPAHRPLLRDPGPRRSESPPGPGGDASLLARLFEHPLYRVAVPPLTEEDVLFNVNSDTRLSPKAAENPDWPHAGAEGAEFLSPGEAAVDSYPNWLKFHIGINRYELYSRHNPAIEALLHDLSSQRITSVAMKSGGTQLKLIMTFQNYGQALFKPMKQTREQETPPDFFYFSDYERHNAEIAAFHLDRILDFRRVPPVAGRMVNMTKEIRDVTRDKKLWRTFFISPANNICFYGECSYYCSTEHALCGKPDQIEGSLAAFLPDLSLAKRKTWRNPWRRSYHKRKKAEWEVDPDYCEEVKQTPPYDSSHRILDVMDMTIFDFLMGNMDRHHYETFEKFGNETFIIHLDNGRGFGKYSHDELSILVPLQQCCRIRKSTYLRLQLLAKEEYKLSLLMAESLRGDQVAPVLYQPHLEALDRRLRVVLKAVRDCVERNGLHSVVDDDLDTEHRAASARDYKDDDDK

SEQ ID NO: 120 - protein, 571 aa, synthetic construct
MLALRRFILNQRSLRSCTIPILVGALIIILVLFQLVTHRNDARGRPGEPPAASSAAGDAGWPNKHTLRILQDFSSDPSSNLSSHSLEKLPPAAEPAERALRGRDPGALRPHDPAHRPLLRDPGPRRSESPPGPGGDASLLARLFEHPLYRVAVPPLTEEDVLFNVNSDTRLSPKAAENPDWPHAGAEGAEFLSPGEAAVDSYPNWLKFHIGINRYELYSRHNPAIEALLHDLSSQRITSVAMKSGGTQLKLIMTFQNYGQALFKPMKQTREQETPPDFFYFSDYERHNAEIAAFHLDRILDFRRVPPVAGRMVNMTKEIRDVTRDKKLWRTFFISPANNICFYGECSYYCSTEHALCGKPDQIEGSLAAFLPDLSLAKRKTWRNPWRRSYHKRKKAEWEVDPDYCEEVKQTPPYDSSHRILDVMDMTIFDFLMGNMDRHHYETFEKFGNETFIIHLDNGRGFGKYSHDELSILVPLQQCCRIRKSTYLRLQLLAKEEYKLSLLMAESLRGDQVAPVLYQPHLEALDRRLRVVLKAVRDCVERNGLHSVVDDDLDTEHRAASARDYKDDDDK

SEQ ID NO: 121 - protein, 622 aa, synthetic construct
MLALRRFILNQRSLRSCTIPILVGALIIILVLFQLVTHRNDALIRSSNVNSTNKKTLKDADPKVLIEAFGSPEVDPVDTIPVSPLELVPFYDQRGRPGEPPAASSAAGDAGWPNKHTLRILQDFSSDPSSNLSSHSLEKLPPAAEPAERALRGRDPGALRPHDPAHRPLLRDPGPRRSESPPGPGGDASLLARLFEHPLYRVAVPPLTEEDVLFNVNSDTRLSPKAAENPDWPHAGAEGAEFLSPGEAAVDSYPNWLKFHIGINRYELYSRHNPAIEALLHDLSSQRITSVAMKSGGTQLKLIMTFQNYGQALFKPMKQTREQETPPDFFYFSDYERHNAEIAAFHLDRILDFRRVPPVAGRMVNMTKEIRDVTRDKKLWRTFFISPANNICFYGECSYYCSTEHALCGKPDQIEGSLAAFLPDLSLAKRKTWRNPWRRSYHKRKKAEWEVDPDYCEEVKQTPPYDSSHRILDVMDMTIFDFLMGNMDRHHYETFEKFGNETFIIHLDNGRGFGKYSHDELSILVPLQQCCRIRKSTYLRLQLLAKEEYKLSLLMAESLRGDQVAPVLYQPHLEALDRRLRVVLKAVRDCVERNGLHSVVDDDLDTEHRAASARDYKDDDDK

SEQ ID NO: 122 - protein, 682 aa, synthetic construct
MLALRRFILNQRSLRSCTIPILVGALIIILVLFQLVTHRNDALIRSSNVNSTNKKTLKDADPKVLIEAFGSPEVDPVDTIPVSPLELVPFYDQSIDTKRSSSWLINKKGYYKHFNELSLTDRCKFYFRTLYTLDDEWTNSVKKLEYSINDNEGRGRPGEPPAASSAAGDAGWPNKHTLRILQDFSSDPSSNLSSHSLEKLPPAAEPAERALRGRDPGALRPHDPAHRPLLRDPGPRRSESPPGPGGDASLLARLFEHPLYRVAVPPLTEEDVLFNVNSDTRLSPKAAENPDWPHAGAEGAEFLSPGEAAVDSYPNWLKFHIGINRYELYSRHNPAIEALLHDLSSQRITSVAMKSGGTQLKLIMTFQNYGQALFKPMKQTREQETPPDFFYFSDYERHNAEIAAFHLDRILDFRRVPPVAGRMVNMTKEIRDVTRDKKLWRTFFISPANNICFYGECSYYCSTEHALCGKPDQIEGSLAAFLPDLSLAKRKTWRNPWRRSYHKRKKAEWEVDPDYCEEVKQTPPYDSSHRILDVMDMTIFDFLMGNMDRHHYETFEKFGNETFIIHLDNGRGFGKYSHDELSILVPLQQCCRIRKSTYLRLQLLAKEEYKLSLLMAESLRGDQVAPVLYQPHLEALDRRLRVVLKAVRDCVERNGLHSVVDDDLDTEHRAASARDYKDDDDK

SEQ ID NO: 123 - protein, 559 aa, synthetic construct
MHVLLSKKIARFLLISFVFVLALMVTINHPRGRPGEPPAASSAAGDAGWPNKHTLRILQDFSSDPSSNLSSHSLEKLPPAAEPAERALRGRDPGALRPHDPAHRPLLRDPGPRRSESPPGPGGDASLLARLFEHPLYRVAVPPLTEEDVLFNVNSDTRLSPKAAENPDWPHAGAEGAEFLSPGEAAVDSYPNWLKFHIGINRYELYSRHNPAIEALLHDLSSQRITSVAMKSGGTQLKLIMTFQNYGQALFKPMKQTREQETPPDFFYFSDYERHNAEIAAFHLDRILDFRRVPPVAGRMVNMTKEIRDVTRDKKLWRTFFISPANNICFYGECSYYCSTEHALCGKPDQIEGSLAAFLPDLSLAKRKTWRNPWRRSYHKRKKAEWEVDPDYCEEVKQTPPYDSSHRILDVMDMTIFDFLMGNMDRHHYETFEKFGNETFIIHLDNGRGFGKYSHDELSILVPLQQCCRIRKSTYLRLQLLAKEEYKLSLLMAESLRGDQVAPVLYQPHLEALDRRLRVVLKAVRDCVERNGLHSVVDDDLDTEHRAASARDYKDDDDK

SEQ ID NO: 124 - protein, 614 aa, synthetic construct
MHVLLSKKIARFLLISFVFVLALMVTINHPKTKQMSEQYVTPYLPKSLQPIAKISAEEQRRIQSEQEEAELKQSLEGEAIRNATVRGRPGEPPAASSAAGDAGWPNKHTLRILQDFSSDPSSNLSSHSLEKLPPAAEPAERALRGRDPGALRPHDPAHRPLLRDPGPRRSESPPGPGGDASLLARLFEHPLYRVAVPPLTEEDVLFNVNSDTRLSPKAAENPDWPHAGAEGAEFLSPGEAAVDSYPNWLKFHIGINRYELYSRHNPAIEALLHDLSSQRITSVAMKSGGTQLKLIMTFQNYGQALFKPMKQTREQETPPDFFYFSDYERHNAEIAAFHLDRILDFRRVPPVAGRMVNMTKEIRDVTRDKKLWRTFFISPANNICFYGECSYYCSTEHALCGKPDQIEGSLAAFLPDLSLAKRKTWRNPWRRSYHKRKKAEWEVDPDYCEEVKQTPPYDSSHRILDVMDMTIFDFLMGNMDRHHYETFEKFGNETFIIHLDNGRGFGKYSHDELSILVPLQQCCRIRKSTYLRLQLLAKEEYKLSLLMAESLRGDQVAPVLYQPHLEALDRRLRVVLKAVRDCVERNGLHSVVDDDLDTEHRAASARDYKDDDDK

SEQ ID NO: 125 - protein, 689 aa, synthetic construct
MHVLLSKKIARFLLISFVFVLALMVTINHPKTKQMSEQYVTPYLPKSLQPIAKISAEEQRRIQSEQEEAELKQSLEGEAIRNATVNAIKEKIKSYGGNETTLGFMVPSYINHRGSPPKACFVSLITERDSMTQILQSIDEVQVKFNKNFAYPWVFISQGERGRPGEPPAASSAAGDAGWPNKHTLRILQDFSSDPSSNLSSHSLEKLPPAAEPAERALRGRDPGALRPHDPAHRPLLRDPGPRRSESPPGPGGDASLLARLFEHPLYRVAVPPLTEEDVLFNVNSDTRLSPKAAENPDWPHAGAEGAEFLSPGEAAVDSYPNWLKFHIGINRYELYSRHNPAIEALLHDLSSQRITSVAMKSGGTQLKLIMTFQNYGQALFKPMKQTREQETPPDFFYFSDYERHNAEIAAFHLDRILDFRRVPPVAGRMVNMTKEIRDVTRDKKLWRTFFISPANNICFYGECSYYCSTEHALCGKPDQIEGSLAAFLPDLSLAKRKTWRNPWRRSYHKRKKAEWEVDPDYCEEVKQTPPYDSSHRILDVMDMTIFDFLMGNMDRHHYETFEKFGNETFIIHLDNGRGFGKYSHDELSILVPLQQCCRIRKSTYLRLQLLAKEEYKLSLLMAESLRGDQVAPVLYQPHLEALDRRLRVVLKAVRDCVERNGLHSVVDDDLDTEHRAASARDYKDDDDK

SEQ ID NO: 126 - protein, 501 aa, synthetic construct
MDFSSDPSSNLSSHSLEKLPPAAEPAERALRGRDPGALRPHDPAHRPLLRDPGPRRSESPPGPGGDASLLARLFEHPLYRVAVPPLTEEDVLFNVNSDTRLSPKAAENPDWPHAGAEGAEFLSPGEAAVDSYPNWLKFHIGINRYELYSRHNPAIEALLHDLSSQRITSVAMKSGGTQLKLIMTFQNYGQALFKPMKQTREQETPPDFFYFSDYERHNAEIAAFHLDRILDFRRVPPVAGRMVNMTKEIRDVTRDKKLWRTFFISPANNICFYGECSYYCSTEHALCGKPDQIEGSLAAFLPDLSLAKRKTWRNPWRRSYHKRKKAEWEVDPDYCEEVKQTPPYDSSHRILDVMDMTIFDFLMGNMDRHHYETFEKFGNETFIIHLDNGRGFGKYSHDELSILVPLQQCCRIRKSTYLRLQLLAKEEYKLSLLMAESLRGDQVAPVLYQPHLEALDRRLRVVLKAVRDCVERNGLHSVVDDDLDTEHRAASARDYKDDDDK

SEQ ID NO: 127 - protein, 558 aa, synthetic construct
MALFLSKRLLRFTVIAGAVIVLLLTLNSNSRTQQYIPSSISAAFDFTSGSISPEQQVIDFSSDPSSNLSSHSLEKLPPAAEPAERALRGRDPGALRPHDPAHRPLLRDPGPRRSESPPGPGGDASLLARLFEHPLYRVAVPPLTEEDVLFNVNSDTRLSPKAAENPDWPHAGAEGAEFLSPGEAAVDSYPNWLKFHIGINRYELYSRHNPAIEALLHDLSSQRITSVAMKSGGTQLKLIMTFQNYGQALFKPMKQTREQETPPDFFYFSDYERHNAEIAAFHLDRILDFRRVPPVAGRMVNMTKEIRDVTRDKKLWRTFFISPANNICFYGECSYYCSTEHALCGKPDQIEGSLAAFLPDLSLAKRKTWRNPWRRSYHKRKKAEWEVDPDYCEEVKQTPPYDSSHRILDVMDMTIFDFLMGNMDRHHYETFEKFGNETFIIHLDNGRGFGKYSHDELSILVPLQQCCRIRKSTYLRLQLLAKEEYKLSLLMAESLRGDQVAPVLYQPHLEALDRRLRVVLKAVRDCVERNGLHSVVDDDLDTEHRAASARDYKDDDDK

SEQ ID NO: 128 - protein, 580 aa, synthetic construct
MALFLSKRLLRFTVIAGAVIVLLLTLNSNSRTQQYIPSSISAAFDFTSGSISPEQQVISEENDAKKLEQSALNSEASEDSDFSSDPSSNLSSHSLEKLPPAAEPAERALRGRDPGALRPHDPAHRPLLRDPGPRRSESPPGPGGDASLLARLFEHPLYRVAVPPLTEEDVLFNVNSDTRLSPKAAENPDWPHAGAEGAEFLSPGEAAVDSYPNWLKFHIGINRYELYSRHNPAIEALLHDLSSQRITSVAMKSGGTQLKLIMTFQNYGQALFKPMKQTREQETPPDFFYFSDYERHNAEIAAFHLDRILDFRRVPPVAGRMVNMTKEIRDVTRDKKLWRTFFISPANNICFYGECSYYCSTEHALCGKPDQIEGSLAAFLPDLSLAKRKTWRNPWRRSYHKRKKAEWEVDPDYCEEVKQTPPYDSSHRILDVMDMTIFDFLMGNMDRHHYETFEKFGNETFIIHLDNGRGFGKYSHDELSILVPLQQCCRIRKSTYLRLQLLAKEEYKLSLLMAESLRGDQVAPVLYQPHLEALDRRLRVVLKAVRDCVERNGLHSVVDDDLDTEHRAASARDYKDDDDK

SEQ ID NO: 129 - protein, 602 aa, synthetic construct
MALFLSKRLLRFTVIAGAVIVLLLTLNSNSRTQQYIPSSISAAFDFTSGSISPEQQVISEENDAKKLEQSALNSEASEDSEAMDEESKALKAAAEKADAPIDDFSSDPSSNLSSHSLEKLPPAAEPAERALRGRDPGALRPHDPAHRPLLRDPGPRRSESPPGPGGDASLLARLFEHPLYRVAVPPLTEEDVLFNVNSDTRLSPKAAENPDWPHAGAEGAEFLSPGEAAVDSYPNWLKFHIGINRYELYSRHNPAIEALLHDLSSQRITSVAMKSGGTQLKLIMTFQNYGQALFKPMKQTREQETPPDFFYFSDYERHNAEIAAFHLDRILDFRRVPPVAGRMVNMTKEIRDVTRDKKLWRTFFISPANNICFYGECSYYCSTEHALCGKPDQIEGSLAAFLPDLSLAKRKTWRNPWRRSYHKRKKAEWEVDPDYCEEVKQTPPYDSSHRILDVMDMTIFDFLMGNMDRHHYETFEKFGNETFIIHLDNGRGFGKYSHDELSILVPLQQCCRIRKSTYLRLQLLAKEEYKLSLLMAESLRGDQVAPVLYQPHLEALDRRLRVVLKAVRDCVERNGLHSVVDDDLDTEHRAASARDYKDDDDK

SEQ ID NO: 130 - protein, 531 aa, synthetic construct
MVHIGFRSLKAVFILALSSLILYGIVTTFDGDFSSDPSSNLSSHSLEKLPPAAEPAERALRGRDPGALRPHDPAHRPLLRDPGPRRSESPPGPGGDASLLARLFEHPLYRVAVPPLTEEDVLFNVNSDTRLSPKAAENPDWPHAGAEGAEFLSPGEAAVDSYPNWLKFHIGINRYELYSRHNPAIEALLHDLSSQRITSVAMKSGGTQLKLIMTFQNYGQALFKPMKQTREQETPPDFFYFSDYERHNAEIAAFHLDRILDFRRVPPVAGRMVNMTKEIRDVTRDKKLWRTFFISPANNICFYGECSYYCSTEHALCGKPDQIEGSLAAFLPDLSLAKRKTWRNPWRRSYHKRKKAEWEVDPDYCEEVKQTPPYDSSHRILDVMDMTIFDFLMGNMDRHHYETFEKFGNETFIIHLDNGRGFGKYSHDELSILVPLQQCCRIRKSTYLRLQLLAKEEYKLSLLMAESLRGDQVAPVLYQPHLEALDRRLRVVLKAVRDCVERNGLHSVVDDDLDTEHRAASARDYKDDDDK

SEQ ID NO: 131 - protein, 584 aa, synthetic construct
MVHIGFRSLKAVFILALSSLILYGIVTTFDGSRASRYQPPYVNHSQDPLYHSGNSYNRENATFVTLCRNEDLYSIIQSIKKVEDDFSSDPSSNLSSHSLEKLPPAAEPAERALRGRDPGALRPHDPAHRPLLRDPGPRRSESPPGPGGDASLLARLFEHPLYRVAVPPLTEEDVLFNVNSDTRLSPKAAENPDWPHAGAEGAEFLSPGEAAVDSYPNWLKFHIGINRYELYSRHNPAIEALLHDLSSQRITSVAMKSGGTQLKLIMTFQNYGQALFKPMKQTREQETPPDFFYFSDYERHNAEIAAFHLDRILDFRRVPPVAGRMVNMTKEIRDVTRDKKLWRTFFISPANNICFYGECSYYCSTEHALCGKPDQIEGSLAAFLPDLSLAKRKTWRNPWRRSYHKRKKAEWEVDPDYCEEVKQTPPYDSSHRILDVMDMTIFDFLMGNMDRHHYETFEKFGNETFIIHLDNGRGFGKYSHDELSILVPLQQCCRIRKSTYLRLQLLAKEEYKLSLLMAESLRGDQVAPVLYQPHLEALDRRLRVVLKAVRDCVERNGLHSVVDDDLDTEHRAASARDYKDDDDK

SEQ ID NO: 132 - protein, 650 aa, synthetic construct
MVHIGFRSLKAVFILALSSLILYGIVTTFDGSRASRYQPPYVNHSQDPLYHSGNSYNRENATFVTLCRNEDLYSIIQSIKKVEDRFNNKFAYDWVFLNEVPFTDEFKERTSVLISGQAKYGLIPKEHWSYPDYIDQERAAESRRQLEDQHDFSSDPSSNLSSHSLEKLPPAAEPAERALRGRDPGALRPHDPAHRPLLRDPGPRRSESPPGPGGDASLLARLFEHPLYRVAVPPLTEEDVLFNVNSDTRLSPKAAENPDWPHAGAEGAEFLSPGEAAVDSYPNWLKFHIGINRYELYSRHNPAIEALLHDLSSQRITSVAMKSGGTQLKLIMTFQNYGQALFKPMKQTREQETPPDFFYFSDYERHNAEIAAFHLDRILDFRRVPPVAGRMVNMTKEIRDVTRDKKLWRTFFISPANNICFYGECSYYCSTEHALCGKPDQIEGSLAAFLPDLSLAKRKTWRNPWRRSYHKRKKAEWEVDPDYCEEVKQTPPYDSSHRILDVMDMTIFDFLMGNMDRHHYETFEKFGNETFIIHLDNGRGFGKYSHDELSILVPLQQCCRIRKSTYLRLQLLAKEEYKLSLLMAESLRGDQVAPVLYQPHLEALDRRLRVVLKAVRDCVERNGLHSVVDDDLDTEHRAASARDYKDDDDK

SEQ ID NO: 133 - protein, 536 aa, synthetic construct
MLLTKRFSKLFKLTFIVLILCGLFVITNKYMDENTSDFSSDPSSNLSSHSLEKLPPAAEPAERALRGRDPGALRPHDPAHRPLLRDPGPRRSESPPGPGGDASLLARLFEHPLYRVAVPPLTEEDVLFNVNSDTRLSPKAAENPDWPHAGAEGAEFLSPGEAAVDSYPNWLKFHIGINRYELYSRHNPAIEALLHDLSSQRITSVAMKSGGTQLKLIMTFQNYGQALFKPMKQTREQETPPDFFYFSDYERHNAEIAAFHLDRILDFRRVPPVAGRMVNMTKEIRDVTRDKKLWRTFFISPANNICFYGECSYYCSTEHALCGKPDQIEGSLAAFLPDLSLAKRKTWRNPWRRSYHKRKKAEWEVDPDYCEEVKQTPPYDSSHRILDVMDMTIFDFLMGNMDRHHYETFEKFGNETFIIHLDNGRGFGKYSHDELSILVPLQQCCRIRKSTYLRLQLLAKEEYKLSLLMAESLRGDQVAPVLYQPHLEALDRRLRVVLKAVRDCVERNGLHSVVDDDLDTEHRAASARDYKDDDDK

SEQ ID NO: 134 - protein, 597 aa, synthetic construct
MLLTKRFSKLFKLTFIVLILCGLFVITNKYMDENTSVKEYKEYLDRYVQSYSNKYSSSSDAASADDSTPLRDNDEAGNEKLKSFYNNVFNFLMVDSPDFSSDPSSNLSSHSLEKLPPAAEPAERALRGRDPGALRPHDPAHRPLLRDPGPRRSESPPGPGGDASLLARLFEHPLYRVAVPPLTEEDVLFNVNSDTRLSPKAAENPDWPHAGAEGAEFLSPGEAAVDSYPNWLKFHIGINRYELYSRHNPAIEALLHDLSSQRITSVAMKSGGTQLKLIMTFQNYGQALFKPMKQTREQETPPDFFYFSDYERHNAEIAAFHLDRILDFRRVPPVAGRMVNMTKEIRDVTRDKKLWRTFFISPANNICFYGECSYYCSTEHALCGKPDQIEGSLAAFLPDLSLAKRKTWRNPWRRSYHKRKKAEWEVDPDYCEEVKQTPPYDSSHRILDVMDMTIFDFLMGNMDRHHYETFEKFGNETFIIHLDNGRGFGKYSHDELSILVPLQQCCRIRKSTYLRLQLLAKEEYKLSLLMAESLRGDQVAPVLYQPHLEALDRRLRVVLKAVRDCVERNGLHSVVDDDLDTEHRAASARDYKDDDDK

SEQ ID NO: 135 - protein, 650 aa, synthetic construct
MLLTKRFSKLFKLTFIVLILCGLFVITNKYMDENTSVKEYKEYLDRYVQSYSNKYSSSSDAASADDSTPLRDNDEAGNEKLKSFYNNVFNFLMVDSPKGSTAKQYNEACLLKGDIGDRPDHYKDLYKLSAKELSKCLELSPDEVASLTKSDFSSDPSSNLSSHSLEKLPPAAEPAERALRGRDPGALRPHDPAHRPLLRDPGPRRSESPPGPGGDASLLARLFEHPLYRVAVPPLTEEDVLFNVNSDTRLSPKAAENPDWPHAGAEGAEFLSPGEAAVDSYPNWLKFHIGINRYELYSRHNPAIEALLHDLSSQRITSVAMKSGGTQLKLIMTFQNYGQALFKPMKQTREQETPPDFFYFSDYERHNAEIAAFHLDRILDFRRVPPVAGRMVNMTKEIRDVTRDKKLWRTFFISPANNICFYGECSYYCSTEHALCGKPDQIEGSLAAFLPDLSLAKRKTWRNPWRRSYHKRKKAEWEVDPDYCEEVKQTPPYDSSHRILDVMDMTIFDFLMGNMDRHHYETFEKFGNETFIIHLDNGRGFGKYSHDELSILVPLQQCCRIRKSTYLRLQLLAKEEYKLSLLMAESLRGDQVAPVLYQPHLEALDRRLRVVLKAVRDCVERNGLHSVVDDDLDTEHRAASARDYKDDDDK

SEQ ID NO: 136 - protein, 542 aa, synthetic construct
MLALRRFILNQRSLRSCTIPILVGALIIILVLFQLVTHRNDADFSSDPSSNLSSHSLEKLPPAAEPAERALRGRDPGALRPHDPAHRPLLRDPGPRRSESPPGPGGDASLLARLFEHPLYRVAVPPLTEEDVLFNVNSDTRLSPKAAENPDWPHAGAEGAEFLSPGEAAVDSYPNWLKFHIGINRYELYSRHNPAIEALLHDLSSQRITSVAMKSGGTQLKLIMTFQNYGQALFKPMKQTREQETPPDFFYFSDYERHNAEIAAFHLDRILDFRRVPPVAGRMVNMTKEIRDVTRDKKLWRTFFISPANNICFYGECSYYCSTEHALCGKPDQIEGSLAAFLPDLSLAKRKTWRNPWRRSYHKRKKAEWEVDPDYCEEVKQTPPYDSSHRILDVMDMTIFDFLMGNMDRHHYETFEKFGNETFIIHLDNGRGFGKYSHDELSILVPLQQCCRIRKSTYLRLQLLAKEEYKLSLLMAESLRGDQVAPVLYQPHLEALDRRLRVVLKAVRDCVERNGLHSVVDDDLDTEHRAASARDYKDDDDK

SEQ ID NO: 137 - protein, 593 aa, synthetic construct
MLALRRFILNQRSLRSCTIPILVGALIIILVLFQLVTHRNDALIRSSNVNSTNKKTLKDADPKVLIEAFGSPEVDPVDTIPVSPLELVPFYDQDFSSDPSSNLSSHSLEKLPPAAEPAERALRGRDPGALRPHDPAHRPLLRDPGPRRSESPPGPGGDASLLARLFEHPLYRVAVPPLTEEDVLFNVNSDTRLSPKAAENPDWPHAGAEGAEFLSPGEAAVDSYPNWLKFHIGINRYELYSRHNPAIEALLHDLSSQRITSVAMKSGGTQLKLIMTFQNYGQALFKPMKQTREQETPPDFFYFSDYERHNAEIAAFHLDRILDFRRVPPVAGRMVNMTKEIRDVTRDKKLWRTFFISPANNICFYGECSYYCSTEHALCGKPDQIEGSLAAFLPDLSLAKRKTWRNPWRRSYHKRKKAEWEVDPDYCEEVKQTPPYDSSHRILDVMDMTIFDFLMGNMDRHHYETFEKFGNETFIIHLDNGRGFGKYSHDELSILVPLQQCCRIRKSTYLRLQLLAKEEYKLSLLMAESLRGDQVAPVLYQPHLEALDRRLRVVLKAVRDCVERNGLHSVVDDDLDTEHRAASARDYKDDDDK

SEQ ID NO: 138 - protein, 653 aa, synthetic construct
MLALRRFILNQRSLRSCTIPILVGALIIILVLFQLVTHRNDALIRSSNVNSTNKKTLKDADPKVLIEAFGSPEVDPVDTIPVSPLELVPFYDQSIDTKRSSSWLINKKGYYKHFNELSLTDRCKFYFRTLYTLDDEWTNSVKKLEYSINDNEGDFSSDPSSNLSSHSLEKLPPAAEPAERALRGRDPGALRPHDPAHRPLLRDPGPRRSESPPGPGGDASLLARLFEHPLYRVAVPPLTEEDVLFNVNSDTRLSPKAAENPDWPHAGAEGAEFLSPGEAAVDSYPNWLKFHIGINRYELYSRHNPAIEALLHDLSSQRITSVAMKSGGTQLKLIMTFQNYGQALFKPMKQTREQETPPDFFYFSDYERHNAEIAAFHLDRILDFRRVPPVAGRMVNMTKEIRDVTRDKKLWRTFFISPANNICFYGECSYYCSTEHALCGKPDQIEGSLAAFLPDLSLAKRKTWRNPWRRSYHKRKKAEWEVDPDYCEEVKQTPPYDSSHRILDVMDMTIFDFLMGNMDRHHYETFEKFGNETFIIHLDNGRGFGKYSHDELSILVPLQQCCRIRKSTYLRLQLLAKEEYKLSLLMAESLRGDQVAPVLYQPHLEALDRRLRVVLKAVRDCVERNGLHSVVDDDLDTEHRAASARDYKDDDDK

SEQ ID NO: 139 - protein, 530 aa, synthetic construct
MHVLLSKKIARFLLISFVFVLALMVTINHPDFSSDPSSNLSSHSLEKLPPAAEPAERALRGRDPGALRPHDPAHRPLLRDPGPRRSESPPGPGGDASLLARLFEHPLYRVAVPPLTEEDVLFNVNSDTRLSPKAAENPDWPHAGAEGAEFLSPGEAAVDSYPNWLKFHIGINRYELYSRHNPAIEALLHDLSSQRITSVAMKSGGTQLKLIMTFQNYGQALFKPMKQTREQETPPDFFYFSDYERHNAEIAAFHLDRILDFRRVPPVAGRMVNMTKEIRDVTRDKKLWRTFFISPANNICFYGECSYYCSTEHALCGKPDQIEGSLAAFLPDLSLAKRKTWRNPWRRSYHKRKKAEWEVDPDYCEEVKQTPPYDSSHRILDVMDMTIFDFLMGNMDRHHYETFEKFGNETFIIHLDNGRGFGKYSHDELSILVPLQQCCRIRKSTYLRLQLLAKEEYKLSLLMAESLRGDQVAPVLYQPHLEALDRRLRVVLKAVRDCVERNGLHSVVDDDLDTEHRAASARDYKDDDDK

SEQ ID NO: 140 - protein, 585 aa, synthetic construct
MHVLLSKKIARFLLISFVFVLALMVTINHPKTKQMSEQYVTPYLPKSLQPIAKISAEEQRRIQSEQEEAELKQSLEGEAIRNATVDFSSDPSSNLSSHSLEKLPPAAEPAERALRGRDPGALRPHDPAHRPLLRDPGPRRSESPPGPGGDASLLARLFEHPLYRVAVPPLTEEDVLFNVNSDTRLSPKAAENPDWPHAGAEGAEFLSPGEAAVDSYPNWLKFHIGINRYELYSRHNPAIEALLHDLSSQRITSVAMKSGGTQLKLIMTFQNYGQALFKPMKQTREQETPPDFFYFSDYERHNAEIAAFHLDRILDFRRVPPVAGRMVNMTKEIRDVTRDKKLWRTFFISPANNICFYGECSYYCSTEHALCGKPDQIEGSLAAFLPDLSLAKRKTWRNPWRRSYHKRKKAEWEVDPDYCEEVKQTPPYDSSHRILDVMDMTIFDFLMGNMDRHHYETFEKFGNETFIIHLDNGRGFGKYSHDELSILVPLQQCCRIRKSTYLRLQLLAKEEYKLSLLMAESLRGDQVAPVLYQPHLEALDRRLRVVLKAVRDCVERNGLHSVVDDDLDTEHRAASARDYKDDDDK

SEQ ID NO: 141 - protein, 660 aa, synthetic construct
MHVLLSKKIARFLLISFVFVLALMVTINHPKTKQMSEQYVTPYLPKSLQPIAKISAEEQRRIQSEQEEAELKQSLEGEAIRNATVNAIKEKIKSYGGNETTLGFMVPSYINHRGSPPKACFVSLITERDSMTQILQSIDEVQVKFNKNFAYPWVFISQGEDFSSDPSSNLSSHSLEKLPPAAEPAERALRGRDPGALRPHDPAHRPLLRDPGPRRSESPPGPGGDASLLARLFEHPLYRVAVPPLTEEDVLFNVNSDTRLSPKAAENPDWPHAGAEGAEFLSPGEAAVDSYPNWLKFHIGINRYELYSRHNPAIEALLHDLSSQRITSVAMKSGGTQLKLIMTFQNYGQALFKPMKQTREQETPPDFFYFSDYERHNAEIAAFHLDRILDFRRVPPVAGRMVNMTKEIRDVTRDKKLWRTFFISPANNICFYGECSYYCSTEHALCGKPDQIEGSLAAFLPDLSLAKRKTWRNPWRRSYHKRKKAEWEVDPDYCEEVKQTPPYDSSHRILDVMDMTIFDFLMGNMDRHHYETFEKFGNETFIIHLDNGRGFGKYSHDELSILVPLQQCCRIRKSTYLRLQLLAKEEYKLSLLMAESLRGDQVAPVLYQPHLEALDRRLRVVLKAVRDCVERNGLHSVVDDDLDTEHRAASARDYKDDDDK

SEQ ID NO: 142 - protein, 553 aa, synthetic construct
RLERRGARPSGEPGCSCAQPAAEVAAPGWAQVRGRPGEPPAASSAAGDAGWPNKHTLRILQDFSSDPSSNLSSHSLEKLPPAAEPAERALRGRDPGALRPHDPAHRPLLRDPGPRRSESPPGPGGDASLLARLFEHPLYRVAVPPLTEEDVLFNVNSDTRLSPKAAENPDWPHAGAEGAEFLSPGEAAVDSYPNWLKFHIGINRYELYSRHNPAIEALLHDLSSQRITSVAMKSGGTQLKLIMTFQNYGQALFKPMKQTREQETPPDFFYFSDYERHNAEIAAFHLDRILDFRRVPPVAGRMVNMTKEIRDVTRDKKLWRTFFISPANNICFYGECSYYCSTEHALCGKPDQIEGSLAAFLPDLSLAKRKTWRNPWRRSYHKRKKAEWEVDPDYCEEVKQTPPYDSSHRILDVMDMTIFDFLMGNMDRHHYETFEKFGNETFIIHLDNGRGFGKYSHDELSILVPLQQCCRIRKSTYLRLQLLAKEEYKLSLLMAESLRGDQVAPVLYQPHLEALDRRLRVVLKAVRDCVERNGLHSVVDDDLDTEHRAASAR

SEQ ID NO: 143 - protein, 521 aa, synthetic construct
RGRPGEPPAASSAAGDAGWPNKHTLRILQDFSSDPSSNLSSHSLEKLPPAAEPAERALRGRDPGALRPHDPAHRPLLRDPGPRRSESPPGPGGDASLLARLFEHPLYRVAVPPLTEEDVLFNVNSDTRLSPKAAENPDWPHAGAEGAEFLSPGEAAVDSYPNWLKFHIGINRYELYSRHNPAIEALLHDLSSQRITSVAMKSGGTQLKLIMTFQNYGQALFKPMKQTREQETPPDFFYFSDYERHNAEIAAFHLDRILDFRRVPPVAGRMVNMTKEIRDVTRDKKLWRTFFISPANNICFYGECSYYCSTEHALCGKPDQIEGSLAAFLPDLSLAKRKTWRNPWRRSYHKRKKAEWEVDPDYCEEVKQTPPYDSSHRILDVMDMTIFDFLMGNMDRHHYETFEKFGNETFIIHLDNGRGFGKYSHDELSILVPLQQCCRIRKSTYLRLQLLAKEEYKLSLLMAESLRGDQVAPVLYQPHLEALDRRLRVVLKAVRDCVERNGLHSVVDDDLDTEHRAASAR

SEQ ID NO: 144 - protein, 492 aa, synthetic construct
DFSSDPSSNLSSHSLEKLPPAAEPAERALRGRDPGALRPHDPAHRPLLRDPGPRRSESPPGPGGDASLLARLFEHPLYRVAVPPLTEEDVLFNVNSDTRLSPKAAENPDWPHAGAEGAEFLSPGEAAVDSYPNWLKFHIGINRYELYSRHNPAIEALLHDLSSQRITSVAMKSGGTQLKLIMTFQNYGQALFKPMKQTREQETPPDFFYFSDYERHNAEIAAFHLDRILDFRRVPPVAGRMVNMTKEIRDVTRDKKLWRTFFISPANNICFYGECSYYCSTEHALCGKPDQIEGSLAAFLPDLSLAKRKTWRNPWRRSYHKRKKAEWEVDPDYCEEVKQTPPYDSSHRILDVMDMTIFDFLMGNMDRHHYETFEKFGNETFIIHLDNGRGFGKYSHDELSILVPLQQCCRIRKSTYLRLQLLAKEEYKLSLLMAESLRGDQVAPVLYQPHLEALDRRLRVVLKAVRDCVERNGLHSVVDDDLDTEHRAASAR

SEQ ID NO: 145 - protein, 584 aa, Homo sapiens; identical to SEQ ID NO: 7

SEQ ID NO: 146 - protein, 592 aa, Homo sapiens; identical to SEQ ID NO: 93

SEQ ID NO: 147 - protein, 594 aa, Homo sapiens
MKMMLVRRFRVLILMVFLVACALHIALDLLPRLERRGARPSGEPGCSCAQPAAEVAAPGWAQVRGRPGEPPAASSAAGDAGWPNKHTLRILQDFSSDPSSNLSSHSLEKLPPAAEPAERALRGRDPGALRPHDPAHRPLLRDPGPRRSESPPGPGGDASLLARLFEHPLYRVAVPPLTEEDVLFNVNSDTRLSPKAAENPDWPHAGAEGAEFLSPGEAAVDSYPNWLKFHIGINRYELYSRHNPAIEALLHDLSSQRITSVAMKSGGTQLKLIMTFQNYGQALFKPMKQTREQETPPDFFYFSDYERHNAEIAAFHLDRILDFRRVPPVAGRMVNMTKEIRDVTRDKKLWRTFFISPANNICFYGECSYYCSTEHALCGKPDQIEGSLAAFLPDLSLAKRKTWRNPWRRSYHKRKKAEWEVDPDYCEEVKQTPPYDSSHRILDVMDMTIFDFLMGNMDRHHYETFEKFGNETFIIHLDNGRGFGKYSHDELSILVPLQQCCRIRKSTYLRLQLLAKEEYKLSLLMAESLRGDQVAPVLYQPHLEALDRRLRVVLKAVRDCVERNGLHSVVDDDLDTEHRAASAREQKLISEEDL

SEQ ID NO: 148 - protein, 583 aa, Homo sapiens
KMMLVRRFRVLILMVFLVACALHIALDLLPRLERRGARPSGEPGCSCAQPAAEVAAPGWAQVRGRPGEPPAASSAAGDAGWPNKHTLRILQDFSSDPSSNLSSHSLEKLPPAAEPAERALRGRDPGALRPHDPAHRPLLRDPGPRRSESPPGPGGDASLLARLFEHPLYRVAVPPLTEEDVLFNVNSDTRLSPKAAENPDWPHAGAEGAEFLSPGEAAVDSYPNWLKFHIGINRYELYSRHNPAIEALLHDLSSQRITSVAMKSGGTQLKLIMTFQNYGQALFKPMKQTREQETPPDFFYFSDYERHNAEIAAFHLDRILDFRRVPPVAGRMVNMTKEIRDVTRDKKLWRTFFISPANNICFYGECSYYCSTEHALCGKPDQIEGSLAAFLPDLSLAKRKTWRNPWRRSYHKRKKAEWEVDPDYCEEVKQTPPYDSSHRILDVMDMTIFDFLMGNMDRHHYETFEKFGNETFIIHLDNGRGFGKYSHDELSILVPLQQCCRIRKSTYLRLQLLAKEEYKLSLLMAESLRGDQVAPVLYQPHLEALDRRLRVVLKAVRDCVERNGLHSVVDDDLDTEHRAASAR

SEQ ID NO: 149 - protein, 22 aa, Homo sapiens
MKMMLVRRFRVLILMVFLVACA

SEQ ID NO: 150 - protein, 70 aa, Homo sapiens
LHIALDLLPRLERRGARPSGEPGCSCAQPAAEVAAPGWAQVRGRPGEPPAASSAAGDAGWPNKHTLRILQ

SEQ ID NO: 151 - protein, 21 aa, Homo sapiens
VLILMVFLVACALHIALDLLP

SEQ ID NO: 152 - protein, 261 aa, Homo sapiens
DFSSDPSSNLSSHSLEKLPPAAEPAERALRGRDPGALRPHDPAHRPLLRDPGPRRSESPPGPGGDASLLARLFEHPLYRVAVPPLTEEDVLFNVNSDTRLSPKAAENPDWPHAGAEGAEFLSPGEAAVDSYPNWLKFHIGINRYELYSRHNPAIEALLHDLSSQRITSVAMKSGGTQLKLIMTFQNYGQALFKPMKQTREQETPPDFFYFSDYERHNAEIAAFHLDRILDFRRVPPVAGRMVNMTKEIRDVTRDKKLWRTF

SEQ ID NO: 153 - protein, 212 aa, Homo sapiens
FISPANNICFYGECSYYCSTEHALCGKPDQIEGSLAAFLPDLSLAKRKTWRNPWRRSYHKRKKAEWEVDPDYCEEVKQTPPYDSSHRILDVMDMTIFDFLMGNMDRHHYETFEKFGNETFIIHLDNGRGFGKYSHDELSILVPLQQCCRIRKSTYLRLQLLAKEEYKLSLLMAESLRGDQVAPVLYQPHLEALDRRLRVVLKAVRDCVERNG

SEQ ID NO: 154 - protein, 594 aa, synthetic construct; identical to SEQ ID NO: 147

SEQ ID NO: 155 - protein, 442 aa, Saccharomyces cerevisiae
MALFLSKRLLRFTVIAGAVIVLLLTLNSNSRTQQYIPSSISAAFDFTSGSISPEQQVISEENDAKKLEQSALNSEASEDSEAMDEESKALKAAAEKADAPIDTKTTMDYITPSFANKAGKPKACYVTLVRNKELKGLLSSIKYVENKINKKFPYPWVFLNDEPFTEEFKEAVTKAVSSEVKFGILPKEHWSYPEWINQTKAAEIRADAATKYIYGGSESYRHMCRYQSGFFWRHELLEEYDWYWRVEPDIKLYCDINYDVFKWMQENEKVYGFTVSIHEYEVTIPTLWQTSMDFIKKNPEYLDENNLMSFLSNDNGKTYNLCHFWSNFEIANLNLWRSPAYREYFDTLDHQGGFFYERWGDAPVHSIAAALFLPKDKIHYFSDIGYHHPPYDNCPLDKEVYNSNNCECDQGNDFTFQGYSCGKEYYDAQGLVKPKNWKKFRE

SEQ ID NO: 156 - protein, 380 aa, unidentified
MVHIGFRSLKAVFILALSSLILYGIVTTFDGSRASRYQPPYVNHSQDPLYHSGNSYNRENATFVTLCRNEDLYSIIQSIKKVEDRFNNKFAYDWVFLNEVPFTDEFKERTSVLISGQAKYGLIPKEHWSYPDYIDQERAAESRRQLEDQHVVYGGLESYRHMCRFNSGFFYKHPLMLDYRYYWRVEPEIEILCDVETDLFRYMRENNKTYGFTISIHEFEKTIPTLWETTKEFMKQNPSYIAENNLMNFISDDNGKTYNLCHFWSNFEVADMDFWRSDVYEKYFKFLDDTGKFFYERWGDAPVHSLAVSLFLPKEKVHFFNEVGYKHSVYSMCPIDKDIWKNRKCYCDPNTDFTFRGYSCGRQYYKATGLTRPSNWKDYD

SEQ ID NO: 157 - protein, 597 aa, Saccharomyces cerevisiae
MLLTKRFSKLFKLTFIVLILCGLFVITNKYMDENTSVKEYKEYLDRYVQSYSNKYSSSSDAASADDSTPLRDNDEAGNEKLKSFYNNVFNFLMVDSPKGSTAKQYNEACLLKGDIGDRPDHYKDLYKLSAKELSKCLELSPDEVASLTKSHKDYVEHIATLVSPKGTYKGSGIATVGGGKFSLMAFLIIKTLRNMGTTLPVEVLIPPGDEGETEFCNKILPKYNSKCIYVSDILPRETIEKFVFKGYQFKSLALIASSFENLLLLDADNFPIKPLDNIFNEEPYVSTGLVMWPDFWRRTTHPLYYDIAGIAVDKKKRVRNSRDDITPPAVYTKDLKDLSDVPLSDLDGTIPDVSTESGQLMINKTKHLATALLSLFYNVNGPTWYYPIFSQKAAGEGDKETFIAAANFYGLSFYQVRTRTGVEGYHDEDGFHGVAMLQHDFVQDYGRYLNAMESIGNKYGGTKSADAIKFDKNYSLEKYTEEFFDNEDLNAKNHVDVMFIHSNFPKFDPYDLSKSNFLTTNGKPARSYTALKKVKNYDIELENFKVLNEYVCVNKNPFKYLDDLLGQDKTEWKRVCGYITDRLAFLESTHDKAIAGK

SEQ ID NO: 158 - protein, 762 aa, Saccharomyces cerevisiae
MLALRRFILNQRSLRSCTIPILVGALIIILVLFQLVTHRNDALIRSSNVNSTNKKTLKDADPKVLIEAFGSPEVDPVDTIPVSPLELVPFYDQSIDTKRSSSWLINKKGYYKHFNELSLTDRCKFYFRTLYTLDDEWTNSVKKLEYSINDNEGVDEGKDANGNPMDEKSERLYRRKYDMFQAFERIRAYDRCFMQANPVNIQEIFPKSDKMSKERVQSKLIKTLNATFPNYDPDNFKKYDQFEFEHKMFPFINNFTTETFHEMVPKITSPFGKVLEQGFLPKFDHKTGKVQEYFKYEYDPSKTFWANWRDMSAKVAGRGIVLSLGSNQFPLAVKFIASLRFEGNTLPIQVVYRGDELSQELVDKLIYAARSPDFKPVENNYDNSTNVPQEIWFLDVSNTIHPKWRGDFGSYKSKWLVVLLNLLQEFVFLDIDAISYEKIDNYFKTTEYQKTGTVFYRERALRENVNERCIARYETLLPRNLESKNFQNSLLIDPDHALNECDNTLTTEEYIFKAFFHHRRQHQLEAGLFAVDKSKHTIPLVLAAMIHLAKNTAHCTHGDKENFWLGFLAAGHTYALQGVYSGAIGDYVKKTDLNGKRQEAAVEICSGQIAHMSTDKKTLLWVNGGGTFCKHDNAAKDDWKKDGDFKKFKDQFKTFEEMEKYYYITPISSKYVILPDPKSDDWHRASAGACGGYIWCATHKTLLKPYSYNHRTTHGELITLDEEQRLHIDAVNTVWSHANKDNTRSFTEEEIKELENSRHEQS

SEQ ID NO: 159 - protein, 446 aa, Saccharomyces cerevisiae
MHVLLSKKIARFLLISFVFVLALMVTINHPKTKQMSEQYVTPYLPKSLQPIAKISAEEQRRIQSEQEEAELKQSLEGEAIRNATVNAIKEKIKSYGGNETTLGFMVPSYINHRGSPPKACFVSLITERDSMTQILQSIDEVQVKFNKNFAYPWVFISQGELDGMKQEMIRQAITDSMNGDPELINIKFAEIPADEWVYPEWIDENKAAESLISLANVPDGDSRAVRYQARYFAGFFWRHPVLDEFDWYWRVDPGIKLYCDIDHDLFRWMQDEGKVFGFTLSMSEAKEANEKIWDVTKKFAKDFPKFISENNFKSFITKKDSEDFNNCEFTSNFEIGNLNFYRSPAYRKFFNYIDEEGGIFYWKWSDSIIHTIGLSMLLPKDKIHFFENIGFHYDKYNNCPLNDDIWNQYNCNCDQGNDFTFRSGSCGGHYFDIMKKDKPEGWDRLP

SEQ ID NO: 160 - protein, 58 aa, synthetic construct; identical to SEQ ID NO: 54

SEQ ID NO: 161 - protein, 80 aa, synthetic construct; identical to SEQ ID NO: 55

SEQ ID NO: 162 - protein, 102 aa, synthetic construct; identical to SEQ ID NO: 56

SEQ ID NO: 163 - protein, 31 aa, synthetic construct; identical to SEQ ID NO: 63

SEQ ID NO: 164 - protein, 84 aa, synthetic construct; identical to SEQ ID NO: 64

SEQ ID NO: 165 - protein, 150 aa, synthetic construct; identical to SEQ ID NO: 65

SEQ ID NO: 166 - protein, 36 aa, synthetic construct; identical to SEQ ID NO: 71

SEQ ID NO: 167 - protein, 97 aa, synthetic construct; identical to SEQ ID NO: 72

SEQ ID NO: 168 - protein, 150 aa, synthetic construct; identical to SEQ ID NO: 73

SEQ ID NO: 169 - protein, 42 aa, synthetic construct; identical to SEQ ID NO: 80

SEQ ID NO: 170 - protein, 93 aa, synthetic construct; identical to SEQ ID NO: 81

SEQ ID NO: 171 - protein, 153 aa, synthetic construct; identical to SEQ ID NO: 82

SEQ ID NO: 172 - protein, 30 aa, synthetic construct; identical to SEQ ID NO: 83

SEQ ID NO: 173 - protein, 85 aa, synthetic construct
MHVLLSKKIARFLLISFVFVLALMVTINHPKTKQMSEQYVTPYLPKSLQPIAKISAEEQRRIQSEQEEAELKQSLEGEAIRNATV

SEQ ID NO: 174 - protein, 160 aa, synthetic construct; identical to SEQ ID NO: 85

SEQ ID NO: 175 - protein, 285 aa, synthetic construct
GDYKDDDDKSDKIIHLTDDSFDTDVLKADGAILVDFWAEWCGPCKMIAPILDEIADEYQGKLTVAKLNIDQNPGTAPKYGIRGIPTLLLFKNGEVAATKVGALSKGQLKEFLDANLAGRGDSVVYGLRSKSKKFRRPDIQYPDATDEDITSHMESEELNGAYKAIPVAQDLNAPSDWDSRGKDSYETSQLDDQSAETHSHKQSRLYKRKANDESNEHSDVIDSQELSKVSREFHSHEFHSHEDMLVVDPKSKEEDKHLKFRISHELDSASSEVNVSGWRLFKKIS

SEQ ID NO: 176 - protein, 405 aa, synthetic construct
GDYKDDDDKGSIGAASMEFCFDVFKELKVHHANENIFYCPIAIMSALAMVYLGAKDSTRTQINKVVRFDKLPGFGDSIEAQCGTSVNVHSSLRDILNQITKPNDVYSFSLASRLYAEERYPILPEYLQCVKELYRGGLEPINFQTAADQARELINSWVESQTNGIIRNVLQPSSVDSQTAMVLVNAIVFKGLWEKAFKDEDTQAMPFRVTEQESKPVQMMYQIGLFRVASMASEKMKILELPFASGTMSMLVLLPDEVSGLEQLESIINFEKLTEWTSSNVMEERKIKVYLPRMKMEEKYNLTSVLMAMGITDVFSSSANLSGISSAESLKISQAVHAAHAEINEAGREVVGSAEAGVDAASVSEEFRADHPFLFCIKHIATNAVLFFGRCVSPVSGWRLFKKIS

SEQ ID NO: 177 - protein, 199 aa, synthetic construct
RPKHPIKHQGLPQEVLNENLLRFFVAPFPEVFGKEKVNELSKDIGSESTEDQAMEDIKQMEAESISSSEEIVPNSVEQKHIQKEDVPSERYLGYLEQLLRLKKYKVPQLEIVPNSAEERLHSMKEGIHAQQKEPMIGVNQELAYFYPELFRQFYQLDAYPSGAWYYVPLGTQYTDAPSFSDIPNPIGSENSGKTTMPLW

SEQ ID NO: 178 - protein, 207 aa, synthetic construct
KNTMEHVSSSEESIISQETYKQEKNMAINPSKENLCSTFCKEVVRNANEEEYSIGSSSEESAEVATEEVKITVDDKHYQKALNEINQFYQKFPQYLQYLYQGPIVLNPWDQVKRNAVPITPTLNREQLSTSEENSKKTVDMESTEVFTKKTKLTEEEKNRLNFLKKISQRYQKFALPQYLKTVYQHQKAMKPWIQPKTKVIPYVRYL

SEQ ID NO: 179 - protein, 209 aa, synthetic construct
RELEELNVPGEIVESLSSSEESITRINKKIEKFQSEEQQQTEDELQDKIHPFAQTQSLVYPFPGPIPNSLPQNIPPLTQTPVVVPPFLQPEVMGVSKVKEAMAPKHKEMPFPKYPVEPFTESQSLTLTDVENLHLPLPLLQSWMHQPHQPLPPTVMFPPQSVLSLSQSKVLPVPQKAVPYPQRDMPIQAFLLYQEPVLGPVRGPFPIIV

SEQ ID NO: 180 - protein, 169 aa, synthetic construct
QEQNQEQPIRCEKDERFFSDKIAKYIPIQYVLSRYPSYGLNYYQQKPVALINNQFLPYPYYAKPAAVRSPAQILQWQVLSNTVPAKSCQAQPTTMARHPHPHLSFMAIPPKKNQDKTEIPTINTIASGEPTSTPTIEAVESTVATLEASPEVIESPPEINTVQVTSTAV

SEQ ID NO: 181 - protein, 19 aa, Saccharomyces cerevisiae
FTVIAGAVIVLLLTLNSNS

SEQ ID NO: 182 - protein, 16 aa, Saccharomyces cerevisiae
LTFIVLILCGLFVITN

SEQ ID NO: 183 - protein, 17 aa, Saccharomyces cerevisiae
CTIPILVGALIIILVLF

SEQ ID NO: 184 - protein, 21 aa, Saccharomyces cerevisiae
IARFLLISFVFVLALMVTINH

SEQ ID NO: 185-310 - skipped (126 entries)

SEQ ID NO: 311 - protein, 10 aa, synthetic construct
EQKLISEEDL

SEQ ID NO: 312 - protein, 85 aa, synthetic construct
MRFPSIFTAVLFAASSALAAPVNTTTEDETAQIPAEAVIGYSDLEGDFDVAVLPFSNSTNNGLLFINTTIASIAAKEEGVSLEKR

SEQ ID NO: 313 - protein, 584 aa, synthetic construct; identical to SEQ ID NO: 7

SEQ ID NO: 314 - protein, 611 aa, synthetic construct
MALFLSKRLLRFTVIAGAVIVLLLTLNSNSRTQQYIPSSISAAFDFTSGSISPEQQVIRLERRGARPSGEPGCSCAQPAAEVAAPGWAQVRGRPGEPPAASSAAGDAGWPNKHTLRILQDFSSDPSSNLSSHSLEKLPPAAEPAERALRGRDPGALRPHDPAHRPLLRDPGPRRSESPPGPGGDASLLARLFEHPLYRVAVPPLTEEDVLFNVNSDTRLSPKAAENPDWPHAGAEGAEFLSPGEAAVDSYPNWLKFHIGINRYELYSRHNPAIEALLHDLSSQRITSVAMKSGGTQLKLIMTFQNYGQALFKPMKQTREQETPPDFFYFSDYERHNAEIAAFHLDRILDFRRVPPVAGRMVNMTKEIRDVTRDKKLWRTFFISPANNICFYGECSYYCSTEHALCGKPDQIEGSLAAFLPDLSLAKRKTWRNPWRRSYHKRKKAEWEVDPDYCEEVKQTPPYDSSHRILDVMDMTIFDFLMGNMDRHHYETFEKFGNETFIIHLDNGRGFGKYSHDELSILVPLQQCCRIRKSTYLRLQLLAKEEYKLSLLMAESLRGDQVAPVLYQPHLEALDRRLRVVLKAVRDCVERNGLHSVVDDDLDTEHRAASAR

SEQ ID NO: 315 - protein, 633 aa, synthetic construct
MALFLSKRLLRFTVIAGAVIVLLLTLNSNSRTQQYIPSSISAAFDFTSGSISPEQQVISEENDAKKLEQSALNSEASEDSRLERRGARPSGEPGCSCAQPAAEVAAPGWAQVRGRPGEPPAASSAAGDAGWPNKHTLRILQDFSSDPSSNLSSHSLEKLPPAAEPAERALRGRDPGALRPHDPAHRPLLRDPGPRRSESPPGPGGDASLLARLFEHPLYRVAVPPLTEEDVLFNVNSDTRLSPKAAENPDWPHAGAEGAEFLSPGEAAVDSYPNWLKFHIGINRYELYSRHNPAIEALLHDLSSQRITSVAMKSGGTQLKLIMTFQNYGQALFKPMKQTREQETPPDFFYFSDYERHNAEIAAFHLDRILDFRRVPPVAGRMVNMTKEIRDVTRDKKLWRTFFISPANNICFYGECSYYCSTEHALCGKPDQIEGSLAAFLPDLSLAKRKTWRNPWRRSYHKRKKAEWEVDPDYCEEVKQTPPYDSSHRILDVMDMTIFDFLMGNMDRHHYETFEKFGNETFIIHLDNGRGFGKYSHDELSILVPLQQCCRIRKSTYLRLQLLAKEEYKLSLLMAESLRGDQVAPVLYQPHLEALDRRLRVVLKAVRDCVERNGLHSVVDDDLDTEHRAASAR

SEQ ID NO: 316 - protein, 655 aa, synthetic construct
MALFLSKRLLRFTVIAGAVIVLLLTLNSNSRTQQYIPSSISAAFDFTSGSISPEQQVISEENDAKKLEQSALNSEASEDSEAMDEESKALKAAAEKADAPIDRLERRGARPSGEPGCSCAQPAAEVAAPGWAQVRGRPGEPPAASSAAGDAGWPNKHTLRILQDFSSDPSSNLSSHSLEKLPPAAEPAERALRGRDPGALRPHDPAHRPLLRDPGPRRSESPPGPGGDASLLARLFEHPLYRVAVPPLTEEDVLFNVNSDTRLSPKAAENPDWPHAGAEGAEFLSPGEAAVDSYPNWLKFHIGINRYELYSRHNPAIEALLHDLSSQRITSVAMKSGGTQLKLIMTFQNYGQALFKPMKQTREQETPPDFFYFSDYERHNAEIAAFHLDRILDFRRVPPVAGRMVNMTKEIRDVTRDKKLWRTFFISPANNICFYGECSYYCSTEHALCGKPDQIEGSLAAFLPDLSLAKRKTWRNPWRRSYHKRKKAEWEVDPDYCEEVKQTPPYDSSHRILDVMDMTIFDFLMGNMDRHHYETFEKFGNETFIIHLDNGRGFGKYSHDELSILVPLQQCCRIRKSTYLRLQLLAKEEYKLSLLMAESLRGDQVAPVLYQPHLEALDRRLRVVLKAVRDCVERNGLHSVVDDDLDTEHRAASAR

SEQ ID NO: 317 - protein, 584 aa, synthetic construct
MVHIGFRSLKAVFILALSSLILYGIVTTFDGRLERRGARPSGEPGCSCAQPAAEVAAPGWAQVRGRPGEPPAASSAAGDAGWPNKHTLRILQDFSSDPSSNLSSHSLEKLPPAAEPAERALRGRDPGALRPHDPAHRPLLRDPGPRRSESPPGPGGDASLLARLFEHPLYRVAVPPLTEEDVLFNVNSDTRLSPKAAENPDWPHAGAEGAEFLSPGEAAVDSYPNWLKFHIGINRYELYSRHNPAIEALLHDLSSQRITSVAMKSGGTQLKLIMTFQNYGQALFKPMKQTREQETPPDFFYFSDYERHNAEIAAFHLDRILDFRRVPPVAGRMVNMTKEIRDVTRDKKLWRTFFISPANNICFYGECSYYCSTEHALCGKPDQIEGSLAAFLPDLSLAKRKTWRNPWRRSYHKRKKAEWEVDPDYCEEVKQTPPYDSSHRILDVMDMTIFDFLMGNMDRHHYETFEKFGNETFIIHLDNGRGFGKYSHDELSILVPLQQCCRIRKSTYLRLQLLAKEEYKLSLLMAESLRGDQVAPVLYQPHLEALDRRLRVVLKAVRDCVERNGLHSVVDDDLDTEHRAASAR

SEQ ID NO: 318 - protein, 637 aa, synthetic construct
MVHIGFRSLKAVFILALSSLILYGIVTTFDGSRASRYQPPYVNHSQDPLYHSGNSYNRENATFVTLCRNEDLYSIIQSIKKVEDRLERRGARPSGEPGCSCAQPAAEVAAPGWAQVRGRPGEPPAASSAAGDAGWPNKHTLRILQDFSSDPSSNLSSHSLEKLPPAAEPAERALRGRDPGALRPHDPAHRPLLRDPGPRRSESPPGPGGDASLLARLFEHPLYRVAVPPLTEEDVLFNVNSDTRLSPKAAENPDWPHAGAEGAEFLSPGEAAVDSYPNWLKFHIGINRYELYSRHNPAIEALLHDLSSQRITSVAMKSGGTQLKLIMTFQNYGQALFKPMKQTREQETPPDFFYFSDYERHNAEIAAFHLDRILDFRRVPPVAGRMVNMTKEIRDVTRDKKLWRTFFISPANNICFYGECSYYCSTEHALCGKPDQIEGSLAAFLPDLSLAKRKTWRNPWRRSYHKRKKAEWEVDPDYCEEVKQTPPYDSSHRILDVMDMTIFDFLMGNMDRHHYETFEKFGNETFIIHLDNGRGFGKYSHDELSILVPLQQCCRIRKSTYLRLQLLAKEEYKLSLLMAESLRGDQVAPVLYQPHLEALDRRLRVVLKAVRDCVERNGLHSVVDDDLDTEHRAASAR

SEQ ID NO: 319 - protein, 703 aa, synthetic construct
MVHIGFRSLKAVFILALSSLILYGIVTTFDGSRASRYQPPYVNHSQDPLYHSGNSYNRENATFVTLCRNEDLYSIIQSIKKVEDRFNNKFAYDWVFLNEVPFTDEFKERTSVLISGQAKYGLIPKEHWSYPDYIDQERAAESRRQLEDQHRLERRGARPSGEPGCSCAQPAAEVAAPGWAQVRGRPGEPPAASSAAGDAGWPNKHTLRILQDFSSDPSSNLSSHSLEKLPPAAEPAERALRGRDPGALRPHDPAHRPLLRDPGPRRSESPPGPGGDASLLARLFEHPLYRVAVPPLTEEDVLFNVNSDTRLSPKAAENPDWPHAGAEGAEFLSPGEAAVDSYPNWLKFHIGINRYELYSRHNPAIEALLHDLSSQRITSVAMKSGGTQLKLIMTFQNYGQALFKPMKQTREQETPPDFFYFSDYERHNAEIAAFHLDRILDFRRVPPVAGRMVNMTKEIRDVTRDKKLWRTFFISPANNICFYGECSYYCSTEHALCGKPDQIEGSLAAFLPDLSLAKRKTWRNPWRRSYHKRKKAEWEVDPDYCEEVKQTPPYDSSHRILDVMDMTIFDFLMGNMDRHHYETFEKFGNETFIIHLDNGRGFGKYSHDELSILVPLQQCCRIRKSTYLRLQLLAKEEYKLSLLMAESLRGDQVAPVLYQPHLEALDRRLRVVLKAVRDCVERNGLHSVVDDDLDTEHRAASAR

SEQ ID NO: 320 - protein, 589 aa, synthetic construct
MLLTKRFSKLFKLTFIVLILCGLFVITNKYMDENTSRLERRGARPSGEPGCSCAQPAAEVAAPGWAQVRGRPGEPPAASSAAGDAGWPNKHTLRILQDFSSDPSSNLSSHSLEKLPPAAEPAERALRGRDPGALRPHDPAHRPLLRDPGPRRSESPPGPGGDASLLARLFEHPLYRVAVPPLTEEDVLFNVNSDTRLSPKAAENPDWPHAGAEGAEFLSPGEAAVDSYPNWLKFHIGINRYELYSRHNPAIEALLHDLSSQRITSVAMKSGGTQLKLIMTFQNYGQALFKPMKQTREQETPPDFFYFSDYERHNAEIAAFHLDRILDFRRVPPVAGRMVNMTKEIRDVTRDKKLWRTFFISPANNICFYGECSYYCSTEHALCGKPDQIEGSLAAFLPDLSLAKRKTWRNPWRRSYHKRKKAEWEVDPDYCEEVKQTPPYDSSHRILDVMDMTIFDFLMGNMDRHHYETFEKFGNETFIIHLDNGRGFGKYSHDELSILVPLQQCCRIRKSTYLRLQLLAKEEYKLSLLMAESLRGDQVAPVLYQPHLEALDRRLRVVLKAVRDCVERNGLHSVVDDDLDTEHRAASAR

SEQ ID NO: 321 - protein, 650 aa, synthetic construct
MLLTKRFSKLFKLTFIVLILCGLFVITNKYMDENTSVKEYKEYLDRYVQSYSNKYSSSSDAASADDSTPLRDNDEAGNEKLKSFYNNVFNFLMVDSPRLERRGARPSGEPGCSCAQPAAEVAAPGWAQVRGRPGEPPAASSAAGDAGWPNKHTLRILQDFSSDPSSNLSSHSLEKLPPAAEPAERALRGRDPGALRPHDPAHRPLLRDPGPRRSESPPGPGGDASLLARLFEHPLYRVAVPPLTEEDVLFNVNSDTRLSPKAAENPDWPHAGAEGAEFLSPGEAAVDSYPNWLKFHIGINRYELYSRHNPAIEALLHDLSSQRITSVAMKSGGTQLKLIMTFQNYGQALFKPMKQTREQETPPDFFYFSDYERHNAEIAAFHLDRILDFRRVPPVAGRMVNMTKEIRDVTRDKKLWRTFFISPANNICFYGECSYYCSTEHALCGKPDQIEGSLAAFLPDLSLAKRKTWRNPWRRSYHKRKKAEWEVDPDYCEEVKQTPPYDSSHRILDVMDMTIFDFLMGNMDRHHYETFEKFGNETFIIHLDNGRGFGKYSHDELSILVPLQQCCRIRKSTYLRLQLLAKEEYKLSLLMAESLRGDQVAPVLYQPHLEALDRRLRVVLKAVRDCVERNGLHSVVDDDLDTEHRAASAR

SEQ ID NO: 322 - protein, 703 aa, synthetic construct
MLLTKRFSKLFKLTFIVLILCGLFVITNKYMDENTSVKEYKEYLDRYVQSYSNKYSSSSDAASADDSTPLRDNDEAGNEKLKSFYNNVFNFLMVDSPKGSTAKQYNEACLLKGDIGDRPDHYKDLYKLSAKELSKCLELSPDEVASLTKSRLERRGARPSGEPGCSCAQPAAEVAAPGWAQVRGRPGEPPAASSAAGDAGWPNKHTLRILQDFSSDPSSNLSSHSLEKLPPAAEPAERALRGRDPGALRPHDPAHRPLLRDPGPRRSESPPGPGGDASLLARLFEHPLYRVAVPPLTEEDVLFNVNSDTRLSPKAAENPDWPHAGAEGAEFLSPGEAAVDSYPNWLKFHIGINRYELYSRHNPAIEALLHDLSSQRITSVAMKSGGTQLKLIMTFQNYGQALFKPMKQTREQETPPDFFYFSDYERHNAEIAAFHLDRILDFRRVPPVAGRMVNMTKEIRDVTRDKKLWRTFFISPANNICFYGECSYYCSTEHALCGKPDQIEGSLAAFLPDLSLAKRKTWRNPWRRSYHKRKKAEWEVDPDYCEEVKQTPPYDSSHRILDVMDMTIFDFLMGNMDRHHYETFEKFGNETFIIHLDNGRGFGKYSHDELSILVPLQQCCRIRKSTYLRLQLLAKEEYKLSLLMAESLRGDQVAPVLYQPHLEALDRRLRVVLKAVRDCVERNGLHSVVDDDLDTEHRAASAR

SEQ ID NO: 323 - protein, 595 aa, synthetic construct
MLALRRFILNQRSLRSCTIPILVGALIIILVLFQLVTHRNDARLERRGARPSGEPGCSCAQPAAEVAAPGWAQVRGRPGEPPAASSAAGDAGWPNKHTLRILQDFSSDPSSNLSSHSLEKLPPAAEPAERALRGRDPGALRPHDPAHRPLLRDPGPRRSESPPGPGGDASLLARLFEHPLYRVAVPPLTEEDVLFNVNSDTRLSPKAAENPDWPHAGAEGAEFLSPGEAAVDSYPNWLKFHIGINRYELYSRHNPAIEALLHDLSSQRITSVAMKSGGTQLKLIMTFQNYGQALFKPMKQTREQETPPDFFYFSDYERHNAEIAAFHLDRILDFRRVPPVAGRMVNMTKEIRDVTRDKKLWRTFFISPANNICFYGECSYYCSTEHALCGKPDQIEGSLAAFLPDLSLAKRKTWRNPWRRSYHKRKKAEWEVDPDYCEEVKQTPPYDSSHRILDVMDMTIFDFLMGNMDRHHYETFEKFGNETFIIHLDNGRGFGKYSHDELSILVPLQQCCRIRKSTYLRLQLLAKEEYKLSLLMAESLRGDQVAPVLYQPHLEALDRRLRVVLKAVRDCVERNGLHSVVDDDLDTEHRAASAR

SEQ ID NO: 324 - protein, 646 aa, synthetic construct
MLALRRFILNQRSLRSCTIPILVGALIIILVLFQLVTHRNDALIRSSNVNSTNKKTLKDADPKVLIEAFGSPEVDPVDTIPVSPLELVPFYDQRLERRGARPSGEPGCSCAQPAAEVAAPGWAQVRGRPGEPPAASSAAGDAGWPNKHTLRILQDFSSDPSSNLSSHSLEKLPPAAEPAERALRGRDPGALRPHDPAHRPLLRDPGPRRSESPPGPGGDASLLARLFEHPLYRVAVPPLTEEDVLFNVNSDTRLSPKAAENPDWPHAGAEGAEFLSPGEAAVDSYPNWLKFHIGINRYELYSRHNPAIEALLHDLSSQRITSVAMKSGGTQLKLIMTFQNYGQALFKPMKQTREQETPPDFFYFSDYERHNAEIAAFHLDRILDFRRVPPVAGRMVNMTKEIRDVTRDKKLWRTFFISPANNICFYGECSYYCSTEHALCGKPDQIEGSLAAFLPDLSLAKRKTWRNPWRRSYHKRKKAEWEVDPDYCEEVKQTPPYDSSHRILDVMDMTIFDFLMGNMDRHHYETFEKFGNETFIIHLDNGRGFGKYSHDELSILVPLQQCCRIRKSTYLRLQLLAKEEYKLSLLMAESLRGDQVAPVLYQPHLEALDRRLRVVLKAVRDCVERNGLHSVVDDDLDTEHRAASAR

SEQ ID NO: 325 - protein, 706 aa, synthetic construct
MLALRRFILNQRSLRSCTIPILVGALIIILVLFQLVTHRNDALIRSSNVNSTNKKTLKDADPKVLIEAFGSPEVDPVDTIPVSPLELVPFYDQSIDTKRSSSWLINKKGYYKHFNELSLTDRCKFYFRTLYTLDDEWTNSVKKLEYSINDNEGRLERRGARPSGEPGCSCAQPAAEVAAPGWAQVRGRPGEPPAASSAAGDAGWPNKHTLRILQDFSSDPSSNLSSHSLEKLPPAAEPAERALRGRDPGALRPHDPAHRPLLRDPGPRRSESPPGPGGDASLLARLFEHPLYRVAVPPLTEEDVLFNVNSDTRLSPKAAENPDWPHAGAEGAEFLSPGEAAVDSYPNWLKFHIGINRYELYSRHNPAIEALLHDLSSQRITSVAMKSGGTQLKLIMTFQNYGQALFKPMKQTREQETPPDFFYFSDYERHNAEIAAFHLDRILDFRRVPPVAGRMVNMTKEIRDVTRDKKLWRTFFISPANNICFYGECSYYCSTEHALCGKPDQIEGSLAAFLPDLSLAKRKTWRNPWRRSYHKRKKAEWEVDPDYCEEVKQTPPYDSSHRILDVMDMTIFDFLMGNMDRHHYETFEKFGNETFIIHLDNGRGFGKYSHDELSILVPLQQCCRIRKSTYLRLQLLAKEEYKLSLLMAESLRGDQVAPVLYQPHLEALDRRLRVVLKAVRDCVERNGLHSVVDDDLDTEHRAASAR

SEQ ID NO: 326 - protein, 583 aa, synthetic construct
MHVLLSKKIARFLLISFVFVLALMVTINHPRLERRGARPSGEPGCSCAQPAAEVAAPGWAQVRGRPGEPPAASSAAGDAGWPNKHTLRILQDFSSDPSSNLSSHSLEKLPPAAEPAERALRGRDPGALRPHDPAHRPLLRDPGPRRSESPPGPGGDASLLARLFEHPLYRVAVPPLTEEDVLFNVNSDTRLSPKAAENPDWPHAGAEGAEFLSPGEAAVDSYPNWLKFHIGINRYELYSRHNPAIEALLHDLSSQRITSVAMKSGGTQLKLIMTFQNYGQALFKPMKQTREQETPPDFFYFSDYERHNAEIAAFHLDRILDFRRVPPVAGRMVNMTKEIRDVTRDKKLWRTFFISPANNICFYGECSYYCSTEHALCGKPDQIEGSLAAFLPDLSLAKRKTWRNPWRRSYHKRKKAEWEVDPDYCEEVKQTPPYDSSHRILDVMDMTIFDFLMGNMDRHHYETFEKFGNETFIIHLDNGRGFGKYSHDELSILVPLQQCCRIRKSTYLRLQLLAKEEYKLSLLMAESLRGDQVAPVLYQPHLEALDRRLRVVLKAVRDCVERNGLHSVVDDDLDTEHRAASAR

SEQ ID NO: 327 - protein, 638 aa, synthetic construct
MHVLLSKKIARFLLISFVFVLALMVTINHPKTKQMSEQYVTPYLPKSLQPIAKISAEEQRRIQSEQEEAELKQSLEGEAIRNATVRLERRGARPSGEPGCSCAQPAAEVAAPGWAQVRGRPGEPPAASSAAGDAGWPNKHTLRILQDFSSDPSSNLSSHSLEKLPPAAEPAERALRGRDPGALRPHDPAHRPLLRDPGPRRSESPPGPGGDASLLARLFEHPLYRVAVPPLTEEDVLFNVNSDTRLSPKAAENPDWPHAGAEGAEFLSPGEAAVDSYPNWLKFHIGINRYELYSRHNPAIEALLHDLSSQRITSVAMKSGGTQLKLIMTFQNYGQALFKPMKQTREQETPPDFFYFSDYERHNAEIAAFHLDRILDFRRVPPVAGRMVNMTKEIRDVTRDKKLWRTFFISPANNICFYGECSYYCSTEHALCGKPDQIEGSLAAFLPDLSLAKRKTWRNPWRRSYHKRKKAEWEVDPDYCEEVKQTPPYDSSHRILDVMDMTIFDFLMGNMDRHHYETFEKFGNETFIIHLDNGRGFGKYSHDELSILVPLQQCCRIRKSTYLRLQLLAKEEYKLSLLMAESLRGDQVAPVLYQPHLEALDRRLRVVLKAVRDCVERNGLHSVVDDDLDTEHRAASAR

SEQ ID NO: 328 - protein, 713 aa, synthetic construct
MHVLLSKKIARFLLISFVFVLALMVTINHPKTKQMSEQYVTPYLPKSLQPIAKISAEEQRRIQSEQEEAELKQSLEGEAIRNATVNAIKEKIKSYGGNETTLGFMVPSYINHRGSPPKACFVSLITERDSMTQILQSIDEVQVKFNKNFAYPWVFISQGERLERRGARPSGEPGCSCAQPAAEVAAPGWAQVRGRPGEPPAASSAAGDAGWPNKHTLRILQDFSSDPSSNLSSHSLEKLPPAAEPAERALRGRDPGALRPHDPAHRPLLRDPGPRRSESPPGPGGDASLLARLFEHPLYRVAVPPLTEEDVLFNVNSDTRLSPKAAENPDWPHAGAEGAEFLSPGEAAVDSYPNWLKFHIGINRYELYSRHNPAIEALLHDLSSQRITSVAMKSGGTQLKLIMTFQNYGQALFKPMKQTREQETPPDFFYFSDYERHNAEIAAFHLDRILDFRRVPPVAGRMVNMTKEIRDVTRDKKLWRTFFISPANNICFYGECSYYCSTEHALCGKPDQIEGSLAAFLPDLSLAKRKTWRNPWRRSYHKRKKAEWEVDPDYCEEVKQTPPYDSSHRILDVMDMTIFDFLMGNMDRHHYETFEKFGNETFIIHLDNGRGFGKYSHDELSILVPLQQCCRIRKSTYLRLQLLAKEEYKLSLLMAESLRGDQVAPVLYQPHLEALDRRLRVVLKAVRDCVERNGLHSVVDDDLDTEHRAASAR

SEQ ID NO: 329 - protein, 522 aa, synthetic construct
MRGRPGEPPAASSAAGDAGWPNKHTLRILQDFSSDPSSNLSSHSLEKLPPAAEPAERALRGRDPGALRPHDPAHRPLLRDPGPRRSESPPGPGGDASLLARLFEHPLYRVAVPPLTEEDVLFNVNSDTRLSPKAAENPDWPHAGAEGAEFLSPGEAAVDSYPNWLKFHIGINRYELYSRHNPAIEALLHDLSSQRITSVAMKSGGTQLKLIMTFQNYGQALFKPMKQTREQETPPDFFYFSDYERHNAEIAAFHLDRILDFRRVPPVAGRMVNMTKEIRDVTRDKKLWRTFFISPANNICFYGECSYYCSTEHALCGKPDQIEGSLAAFLPDLSLAKRKTWRNPWRRSYHKRKKAEWEVDPDYCEEVKQTPPYDSSHRILDVMDMTIFDFLMGNMDRHHYETFEKFGNETFIIHLDNGRGFGKYSHDELSILVPLQQCCRIRKSTYLRLQLLAKEEYKLSLLMAESLRGDQVAPVLYQPHLEALDRRLRVVLKAVRDCVERNGLHSVVDDDLDTEHRAASAR

SEQ ID NO: 330 - protein, 579 aa, synthetic construct
MALFLSKRLLRFTVIAGAVIVLLLTLNSNSRTQQYIPSSISAAFDFTSGSISPEQQVIRGRPGEPPAASSAAGDAGWPNKHTLRILQDFSSDPSSNLSSHSLEKLPPAAEPAERALRGRDPGALRPHDPAHRPLLRDPGPRRSESPPGPGGDASLLARLFEHPLYRVAVPPLTEEDVLFNVNSDTRLSPKAAENPDWPHAGAEGAEFLSPGEAAVDSYPNWLKFHIGINRYELYSRHNPAIEALLHDLSSQRITSVAMKSGGTQLKLIMTFQNYGQALFKPMKQTREQETPPDFFYFSDYERHNAEIAAFHLDRILDFRRVPPVAGRMVNMTKEIRDVTRDKKLWRTFFISPANNICFYGECSYYCSTEHALCGKPDQIEGSLAAFLPDLSLAKRKTWRNPWRRSYHKRKKAEWEVDPDYCEEVKQTPPYDSSHRILDVMDMTIFDFLMGNMDRHHYETFEKFGNETFIIHLDNGRGFGKYSHDELSILVPLQQCCRIRKSTYLRLQLLAKEEYKLSLLMAESLRGDQVAPVLYQPHLEALDRRLRVVLKAVRDCVERNGLHSVVDDDLDTEHRAASAR

SEQ ID NO: 331 - protein, 601 aa, synthetic construct
MALFLSKRLLRFTVIAGAVIVLLLTLNSNSRTQQYIPSSISAAFDFTSGSISPEQQVISEENDAKKLEQSALNSEASEDSRGRPGEPPAASSAAGDAGWPNKHTLRILQDFSSDPSSNLSSHSLEKLPPAAEPAERALRGRDPGALRPHDPAHRPLLRDPGPRRSESPPGPGGDASLLARLFEHPLYRVAVPPLTEEDVLFNVNSDTRLSPKAAENPDWPHAGAEGAEFLSPGEAAVDSYPNWLKFHIGINRYELYSRHNPAIEALLHDLSSQRITSVAMKSGGTQLKLIMTFQNYGQALFKPMKQTREQETPPDFFYFSDYERHNAEIAAFHLDRILDFRRVPPVAGRMVNMTKEIRDVTRDKKLWRTFFISPANNICFYGECSYYCSTEHALCGKPDQIEGSLAAFLPDLSLAKRKTWRNPWRRSYHKRKKAEWEVDPDYCEEVKQTPPYDSSHRILDVMDMTIFDFLMGNMDRHHYETFEKFGNETFIIHLDNGRGFGKYSHDELSILVPLQQCCRIRKSTYLRLQLLAKEEYKLSLLMAESLRGDQVAPVLYQPHLEALDRRLRVVLKAVRDCVERNGLHSVVDDDLDTEHRAASAR

SEQ ID NO: 332 - protein, 623 aa, synthetic construct
MALFLSKRLLRFTVIAGAVIVLLLTLNSNSRTQQYIPSSISAAFDFTSGSISPEQQVISEENDAKKLEQSALNSEASEDSEAMDEESKALKAAAEKADAPIDRGRPGEPPAASSAAGDAGWPNKHTLRILQDFSSDPSSNLSSHSLEKLPPAAEPAERALRGRDPGALRPHDPAHRPLLRDPGPRRSESPPGPGGDASLLARLFEHPLYRVAVPPLTEEDVLFNVNSDTRLSPKAAENPDWPHAGAEGAEFLSPGEAAVDSYPNWLKFHIGINRYELYSRHNPAIEALLHDLSSQRITSVAMKSGGTQLKLIMTFQNYGQALFKPMKQTREQETPPDFFYFSDYERHNAEIAAFHLDRILDFRRVPPVAGRMVNMTKEIRDVTRDKKLWRTFFISPANNICFYGECSYYCSTEHALCGKPDQIEGSLAAFLPDLSLAKRKTWRNPWRRSYHKRKKAEWEVDPDYCEEVKQTPPYDSSHRILDVMDMTIFDFLMGNMDRHHYETFEKFGNETFIIHLDNGRGFGKYSHDELSILVPLQQCCRIRKSTYLRLQLLAKEEYKLSLLMAESLRGDQVAPVLYQPHLEALDRRLRVVLKAVRDCVERNGLHSVVDDDLDTEHRAASAR

SEQ ID NO: 333 - protein, 552 aa, synthetic construct
MVHIGFRSLKAVFILALSSLILYGIVTTFDGRGRPGEPPAASSAAGDAGWPNKHTLRILQDFSSDPSSNLSSHSLEKLPPAAEPAERALRGRDPGALRPHDPAHRPLLRDPGPRRSESPPGPGGDASLLARLFEHPLYRVAVPPLTEEDVLFNVNSDTRLSPKAAENPDWPHAGAEGAEFLSPGEAAVDSYPNWLKFHIGINRYELYSRHNPAIEALLHDLSSQRITSVAMKSGGTQLKLIMTFQNYGQALFKPMKQTREQETPPDFFYFSDYERHNAEIAAFHLDRILDFRRVPPVAGRMVNMTKEIRDVTRDKKLWRTFFISPANNICFYGECSYYCSTEHALCGKPDQIEGSLAAFLPDLSLAKRKTWRNPWRRSYHKRKKAEWEVDPDYCEEVKQTPPYDSSHRILDVMDMTIFDFLMGNMDRHHYETFEKFGNETFIIHLDNGRGFGKYSHDELSILVPLQQCCRIRKSTYLRLQLLAKEEYKLSLLMAESLRGDQVAPVLYQPHLEALDRRLRVVLKAVRDCVERNGLHSVVDDDLDTEHRAASAR

SEQ ID NO: 334 - protein, 605 aa, synthetic construct
MVHIGFRSLKAVFILALSSLILYGIVTTFDGSRASRYQPPYVNHSQDPLYHSGNSYNRENATFVTLCRNEDLYSIIQSIKKVEDRGRPGEPPAASSAAGDAGWPNKHTLRILQDFSSDPSSNLSSHSLEKLPPAAEPAERALRGRDPGALRPHDPAHRPLLRDPGPRRSESPPGPGGDASLLARLFEHPLYRVAVPPLTEEDVLFNVNSDTRLSPKAAENPDWPHAGAEGAEFLSPGEAAVDSYPNWLKFHIGINRYELYSRHNPAIEALLHDLSSQRITSVAMKSGGTQLKLIMTFQNYGQALFKPMKQTREQETPPDFFYFSDYERHNAEIAAFHLDRILDFRRVPPVAGRMVNMTKEIRDVTRDKKLWRTFFISPANNICFYGECSYYCSTEHALCGKPDQIEGSLAAFLPDLSLAKRKTWRNPWRRSYHKRKKAEWEVDPDYCEEVKQTPPYDSSHRILDVMDMTIFDFLMGNMDRHHYETFEKFGNETFIIHLDNGRGFGKYSHDELSILVPLQQCCRIRKSTYLRLQLLAKEEYKLSLLMAESLRGDQVAPVLYQPHLEALDRRLRVVLKAVRDCVERNGLHSVVDDDLDTEHRAASAR

SEQ ID NO: 335 - protein, 671 aa, synthetic construct
MVHIGFRSLKAVFILALSSLILYGIVTTFDGSRASRYQPPYVNHSQDPLYHSGNSYNRENATFVTLCRNEDLYSIIQSIKKVEDRFNNKFAYDWVFLNEVPFTDEFKERTSVLISGQAKYGLIPKEHWSYPDYIDQERAAESRRQLEDQHRGRPGEPPAASSAAGDAGWPNKHTLRILQDFSSDPSSNLSSHSLEKLPPAAEPAERALRGRDPGALRPHDPAHRPLLRDPGPRRSESPPGPGGDASLLARLFEHPLYRVAVPPLTEEDVLFNVNSDTRLSPKAAENPDWPHAGAEGAEFLSPGEAAVDSYPNWLKFHIGINRYELYSRHNPAIEALLHDLSSQRITSVAMKSGGTQLKLIMTFQNYGQALFKPMKQTREQETPPDFFYFSDYERHNAEIAAFHLDRILDFRRVPPVAGRMVNMTKEIRDVTRDKKLWRTFFISPANNICFYGECSYYCSTEHALCGKPDQIEGSLAAFLPDLSLAKRKTWRNPWRRSYHKRKKAEWEVDPDYCEEVKQTPPYDSSHRILDVMDMTIFDFLMGNMDRHHYETFEKFGNETFIIHLDNGRGFGKYSHDELSILVPLQQCCRIRKSTYLRLQLLAKEEYKLSLLMAESLRGDQVAPVLYQPHLEALDRRLRVVLKAVRDCVERNGLHSVVDDDLDTEHRAASAR

SEQ ID NO: 336 - protein, 557 aa, synthetic construct
MLLTKRFSKLFKLTFIVLILCGLFVITNKYMDENTSRGRPGEPPAASSAAGDAGWPNKHTLRILQDFSSDPSSNLSSHSLEKLPPAAEPAERALRGRDPGALRPHDPAHRPLLRDPGPRRSESPPGPGGDASLLARLFEHPLYRVAVPPLTEEDVLFNVNSDTRLSPKAAENPDWPHAGAEGAEFLSPGEAAVDSYPNWLKFHIGINRYELYSRHNPAIEALLHDLSSQRITSVAMKSGGTQLKLIMTFQNYGQALFKPMKQTREQETPPDFFYFSDYERHNAEIAAFHLDRILDFRRVPPVAGRMVNMTKEIRDVTRDKKLWRTFFISPANNICFYGECSYYCSTEHALCGKPDQIEGSLAAFLPDLSLAKRKTWRNPWRRSYHKRKKAEWEVDPDYCEEVKQTPPYDSSHRILDVMDMTIFDFLMGNMDRHHYETFEKFGNETFIIHLDNGRGFGKYSHDELSILVPLQQCCRIRKSTYLRLQLLAKEEYKLSLLMAESLRGDQVAPVLYQPHLEALDRRLRVVLKAVRDCVERNGLHSVVDDDLDTEHRAASAR

SEQ ID NO: 337 - protein, 618 aa, synthetic construct
MLLTKRFSKLFKLTFIVLILCGLFVITNKYMDENTSVKEYKEYLDRYVQSYSNKYSSSSDAASADDSTPLRDNDEAGNEKLKSFYNNVFNFLMVDSPRGRPGEPPAASSAAGDAGWPNKHTLRILQDFSSDPSSNLSSHSLEKLPPAAEPAERALRGRDPGALRPHDPAHRPLLRDPGPRRSESPPGPGGDASLLARLFEHPLYRVAVPPLTEEDVLFNVNSDTRLSPKAAENPDWPHAGAEGAEFLSPGEAAVDSYPNWLKFHIGINRYELYSRHNPAIEALLHDLSSQRITSVAMKSGGTQLKLIMTFQNYGQALFKPMKQTREQETPPDFFYFSDYERHNAEIAAFHLDRILDFRRVPPVAGRMVNMTKEIRDVTRDKKLWRTFFISPANNICFYGECSYYCSTEHALCGKPDQIEGSLAAFLPDLSLAKRKTWRNPWRRSYHKRKKAEWEVDPDYCEEVKQTPPYDSSHRILDVMDMTIFDFLMGNMDRHHYETFEKFGNETFIIHLDNGRGFGKYSHDELSILVPLQQCCRIRKSTYLRLQLLAKEEYKLSLLMAESLRGDQVAPVLYQPHLEALDRRLRVVLKAVRDCVERNGLHSVVDDDLDTEHRAASAR

SEQ ID NO: 338 - protein, 671 aa, synthetic construct
MLLTKRFSKLFKLTFIVLILCGLFVITNKYMDENTSVKEYKEYLDRYVQSYSNKYSSSSDAASADDSTPLRDNDEAGNEKLKSFYNNVFNFLMVDSPKGSTAKQYNEACLLKGDIGDRPDHYKDLYKLSAKELSKCLELSPDEVASLTKSRGRPGEPPAASSAAGDAGWPNKHTLRILQDFSSDPSSNLSSHSLEKLPPAAEPAERALRGRDPGALRPHDPAHRPLLRDPGPRRSESPPGPGGDASLLARLFEHPLYRVAVPPLTEEDVLFNVNSDTRLSPKAAENPDWPHAGAEGAEFLSPGEAAVDSYPNWLKFHIGINRYELYSRHNPAIEALLHDLSSQRITSVAMKSGGTQLKLIMTFQNYGQALFKPMKQTREQETPPDFFYFSDYERHNAEIAAFHLDRILDFRRVPPVAGRMVNMTKEIRDVTRDKKLWRTFFISPANNICFYGECSYYCSTEHALCGKPDQIEGSLAAFLPDLSLAKRKTWRNPWRRSYHKRKKAEWEVDPDYCEEVKQTPPYDSSHRILDVMDMTIFDFLMGNMDRHHYETFEKFGNETFIIHLDNGRGFGKYSHDELSILVPLQQCCRIRKSTYLRLQLLAKEEYKLSLLMAESLRGDQVAPVLYQPHLEALDRRLRVVLKAVRDCVERNGLHSVVDDDLDTEHRAASAR

SEQ ID NO: 339 - protein, 563 aa, synthetic construct
MLALRRFILNQRSLRSCTIPILVGALIIILVLFQLVTHRNDARGRPGEPPAASSAAGDAGWPNKHTLRILQDFSSDPSSNLSSHSLEKLPPAAEPAERALRGRDPGALRPHDPAHRPLLRDPGPRRSESPPGPGGDASLLARLFEHPLYRVAVPPLTEEDVLFNVNSDTRLSPKAAENPDWPHAGAEGAEFLSPGEAAVDSYPNWLKFHIGINRYELYSRHNPAIEALLHDLSSQRITSVAMKSGGTQLKLIMTFQNYGQALFKPMKQTREQETPPDFFYFSDYERHNAEIAAFHLDRILDFRRVPPVAGRMVNMTKEIRDVTRDKKLWRTFFISPANNICFYGECSYYCSTEHALCGKPDQIEGSLAAFLPDLSLAKRKTWRNPWRRSYHKRKKAEWEVDPDYCEEVKQTPPYDSSHRILDVMDMTIFDFLMGNMDRHHYETFEKFGNETFIIHLDNGRGFGKYSHDELSILVPLQQCCRIRKSTYLRLQLLAKEEYKLSLLMAESLRGDQVAPVLYQPHLEALDRRLRVVLKAVRDCVERNGLHSVVDDDLDTEHRAASAR

SEQ ID NO: 340 - protein, 614 aa, synthetic construct
MLALRRFILNQRSLRSCTIPILVGALIIILVLFQLVTHRNDALIRSSNVNSTNKKTLKDADPKVLIEAFGSPEVDPVDTIPVSPLELVPFYDQRGRPGEPPAASSAAGDAGWPNKHTLRILQDFSSDPSSNLSSHSLEKLPPAAEPAERALRGRDPGALRPHDPAHRPLLRDPGPRRSESPPGPGGDASLLARLFEHPLYRVAVPPLTEEDVLFNVNSDTRLSPKAAENPDWPHAGAEGAEFLSPGEAAVDSYPNWLKFHIGINRYELYSRHNPAIEALLHDLSSQRITSVAMKSGGTQLKLIMTFQNYGQALFKPMKQTREQETPPDFFYFSDYERHNAEIAAFHLDRILDFRRVPPVAGRMVNMTKEIRDVTRDKKLWRTFFISPANNICFYGECSYYCSTEHALCGKPDQIEGSLAAFLPDLSLAKRKTWRNPWRRSYHKRKKAEWEVDPDYCEEVKQTPPYDSSHRILDVMDMTIFDFLMGNMDRHHYETFEKFGNETFIIHLDNGRGFGKYSHDELSILVPLQQCCRIRKSTYLRLQLLAKEEYKLSLLMAESLRGDQVAPVLYQPHLEALDRRLRVVLKAVRDCVERNGLHSVVDDDLDTEHRAASAR

SEQ ID NO: 341 - protein, 674 aa, synthetic construct
MLALRRFILNQRSLRSCTIPILVGALIIILVLFQLVTHRNDALIRSSNVNSTNKKTLKDADPKVLIEAFGSPEVDPVDTIPVSPLELVPFYDQSIDTKRSSSWLINKKGYYKHFNELSLTDRCKFYFRTLYTLDDEWTNSVKKLEYSINDNEGRGRPGEPPAASSAAGDAGWPNKHTLRILQDFSSDPSSNLSSHSLEKLPPAAEPAERALRGRDPGALRPHDPAHRPLLRDPGPRRSESPPGPGGDASLLARLFEHPLYRVAVPPLTEEDVLFNVNSDTRLSPKAAENPDWPHAGAEGAEFLSPGEAAVDSYPNWLKFHIGINRYELYSRHNPAIEALLHDLSSQRITSVAMKSGGTQLKLIMTFQNYGQALFKPMKQTREQETPPDFFYFSDYERHNAEIAAFHLDRILDFRRVPPVAGRMVNMTKEIRDVTRDKKLWRTFFISPANNICFYGECSYYCSTEHALCGKPDQIEGSLAAFLPDLSLAKRKTWRNPWRRSYHKRKKAEWEVDPDYCEEVKQTPPYDSSHRILDVMDMTIFDFLMGNMDRHHYETFEKFGNETFIIHLDNGRGFGKYSHDELSILVPLQQCCRIRKSTYLRLQLLAKEEYKLSLLMAESLRGDQVAPVLYQPHLEALDRRLRVVLKAVRDCVERNGLHSVVDDDLDTEHRAASAR

SEQ ID NO: 342 - protein, 551 aa, synthetic construct
MHVLLSKKIARFLLISFVFVLALMVTINHPRGRPGEPPAASSAAGDAGWPNKHTLRILQDFSSDPSSNLSSHSLEKLPPAAEPAERALRGRDPGALRPHDPAHRPLLRDPGPRRSESPPGPGGDASLLARLFEHPLYRVAVPPLTEEDVLFNVNSDTRLSPKAAENPDWPHAGAEGAEFLSPGEAAVDSYPNWLKFHIGINRYELYSRHNPAIEALLHDLSSQRITSVAMKSGGTQLKLIMTFQNYGQALFKPMKQTREQETPPDFFYFSDYERHNAEIAAFHLDRILDFRRVPPVAGRMVNMTKEIRDVTRDKKLWRTFFISPANNICFYGECSYYCSTEHALCGKPDQIEGSLAAFLPDLSLAKRKTWRNPWRRSYHKRKKAEWEVDPDYCEEVKQTPPYDSSHRILDVMDMTIFDFLMGNMDRHHYETFEKFGNETFIIHLDNGRGFGKYSHDELSILVPLQQCCRIRKSTYLRLQLLAKEEYKLSLLMAESLRGDQVAPVLYQPHLEALDRRLRVVLKAVRDCVERNGLHSVVDDDLDTEHRAASAR

SEQ ID NO: 343 - protein, 606 aa, synthetic construct
MHVLLSKKIARFLLISFVFVLALMVTINHPKTKQMSEQYVTPYLPKSLQPIAKISAEEQRRIQSEQEEAELKQSLEGEAIRNATVRGRPGEPPAASSAAGDAGWPNKHTLRILQDFSSDPSSNLSSHSLEKLPPAAEPAERALRGRDPGALRPHDPAHRPLLRDPGPRRSESPPGPGGDASLLARLFEHPLYRVAVPPLTEEDVLFNVNSDTRLSPKAAENPDWPHAGAEGAEFLSPGEAAVDSYPNWLKFHIGINRYELYSRHNPAIEALLHDLSSQRITSVAMKSGGTQLKLIMTFQNYGQALFKPMKQTREQETPPDFFYFSDYERHNAEIAAFHLDRILDFRRVPPVAGRMVNMTKEIRDVTRDKKLWRTFFISPANNICFYGECSYYCSTEHALCGKPDQIEGSLAAFLPDLSLAKRKTWRNPWRRSYHKRKKAEWEVDPDYCEEVKQTPPYDSSHRILDVMDMTIFDFLMGNMDRHHYETFEKFGNETFIIHLDNGRGFGKYSHDELSILVPLQQCCRIRKSTYLRLQLLAKEEYKLSLLMAESLRGDQVAPVLYQPHLEALDRRLRVVLKAVRDCVERNGLHSVVDDDLDTEHRAASAR

SEQ ID NO: 344 - protein, 681 aa, synthetic construct
MHVLLSKKIARFLLISFVFVLALMVTINHPKTKQMSEQYVTPYLPKSLQPIAKISAEEQRRIQSEQEEAELKQSLEGEAIRNATVNAIKEKIKSYGGNETTLGFMVPSYINHRGSPPKACFVSLITERDSMTQILQSIDEVQVKFNKNFAYPWVFISQGERGRPGEPPAASSAAGDAGWPNKHTLRILQDFSSDPSSNLSSHSLEKLPPAAEPAERALRGRDPGALRPHDPAHRPLLRDPGPRRSESPPGPGGDASLLARLFEHPLYRVAVPPLTEEDVLFNVNSDTRLSPKAAENPDWPHAGAEGAEFLSPGEAAVDSYPNWLKFHIGINRYELYSRHNPAIEALLHDLSSQRITSVAMKSGGTQLKLIMTFQNYGQALFKPMKQTREQETPPDFFYFSDYERHNAEIAAFHLDRILDFRRVPPVAGRMVNMTKEIRDVTRDKKLWRTFFISPANNICFYGECSYYCSTEHALCGKPDQIEGSLAAFLPDLSLAKRKTWRNPWRRSYHKRKKAEWEVDPDYCEEVKQTPPYDSSHRILDVMDMTIFDFLMGNMDRHHYETFEKFGNETFIIHLDNGRGFGKYSHDELSILVPLQQCCRIRKSTYLRLQLLAKEEYKLSLLMAESLRGDQVAPVLYQPHLEALDRRLRVVLKAVRDCVERNGLHSVVDDDLDTEHRAASAR

SEQ ID NO: 345 - protein, 493 aa, synthetic construct
MDFSSDPSSNLSSHSLEKLPPAAEPAERALRGRDPGALRPHDPAHRPLLRDPGPRRSESPPGPGGDASLLARLFEHPLYRVAVPPLTEEDVLFNVNSDTRLSPKAAENPDWPHAGAEGAEFLSPGEAAVDSYPNWLKFHIGINRYELYSRHNPAIEALLHDLSSQRITSVAMKSGGTQLKLIMTFQNYGQALFKPMKQTREQETPPDFFYFSDYERHNAEIAAFHLDRILDFRRVPPVAGRMVNMTKEIRDVTRDKKLWRTFFISPANNICFYGECSYYCSTEHALCGKPDQIEGSLAAFLPDLSLAKRKTWRNPWRRSYHKRKKAEWEVDPDYCEEVKQTPPYDSSHRILDVMDMTIFDFLMGNMDRHHYETFEKFGNETFIIHLDNGRGFGKYSHDELSILVPLQQCCRIRKSTYLRLQLLAKEEYKLSLLMAESLRGDQVAPVLYQPHLEALDRRLRVVLKAVRDCVERNGLHSVVDDDLDTEHRAASAR

SEQ ID NO: 346 - protein, 550 aa, synthetic construct
MALFLSKRLLRFTVIAGAVIVLLLTLNSNSRTQQYIPSSISAAFDFTSGSISPEQQVIDFSSDPSSNLSSHSLEKLPPAAEPAERALRGRDPGALRPHDPAHRPLLRDPGPRRSESPPGPGGDASLLARLFEHPLYRVAVPPLTEEDVLFNVNSDTRLSPKAAENPDWPHAGAEGAEFLSPGEAAVDSYPNWLKFHIGINRYELYSRHNPAIEALLHDLSSQRITSVAMKSGGTQLKLIMTFQNYGQALFKPMKQTREQETPPDFFYFSDYERHNAEIAAFHLDRILDFRRVPPVAGRMVNMTKEIRDVTRDKKLWRTFFISPANNICFYGECSYYCSTEHALCGKPDQIEGSLAAFLPDLSLAKRKTWRNPWRRSYHKRKKAEWEVDPDYCEEVKQTPPYDSSHRILDVMDMTIFDFLMGNMDRHHYETFEKFGNETFIIHLDNGRGFGKYSHDELSILVPLQQCCRIRKSTYLRLQLLAKEEYKLSLLMAESLRGDQVAPVLYQPHLEALDRRLRVVLKAVRDCVERNGLHSVVDDDLDTEHRAASAR

SEQ ID NO: 347 - protein, 572 aa, synthetic construct
MALFLSKRLLRFTVIAGAVIVLLLTLNSNSRTQQYIPSSISAAFDFTSGSISPEQQVISEENDAKKLEQSALNSEASEDSDFSSDPSSNLSSHSLEKLPPAAEPAERALRGRDPGALRPHDPAHRPLLRDPGPRRSESPPGPGGDASLLARLFEHPLYRVAVPPLTEEDVLFNVNSDTRLSPKAAENPDWPHAGAEGAEFLSPGEAAVDSYPNWLKFHIGINRYELYSRHNPAIEALLHDLSSQRITSVAMKSGGTQLKLIMTFQNYGQALFKPMKQTREQETPPDFFYFSDYERHNAEIAAFHLDRILDFRRVPPVAGRMVNMTKEIRDVTRDKKLWRTFFISPANNICFYGECSYYCSTEHALCGKPDQIEGSLAAFLPDLSLAKRKTWRNPWRRSYHKRKKAEWEVDPDYCEEVKQTPPYDSSHRILDVMDMTIFDFLMGNMDRHHYETFEKFGNETFIIHLDNGRGFGKYSHDELSILVPLQQCCRIRKSTYLRLQLLAKEEYKLSLLMAESLRGDQVAPVLYQPHLEALDRRLRVVLKAVRDCVERNGLHSVVDDDLDTEHRAASAR

SEQ ID NO: 348 - protein, 594 aa, synthetic construct
MALFLSKRLLRFTVIAGAVIVLLLTLNSNSRTQQYIPSSISAAFDFTSGSISPEQQVISEENDAKKLEQSALNSEASEDSEAMDEESKALKAAAEKADAPIDDFSSDPSSNLSSHSLEKLPPAAEPAERALRGRDPGALRPHDPAHRPLLRDPGPRRSESPPGPGGDASLLARLFEHPLYRVAVPPLTEEDVLFNVNSDTRLSPKAAENPDWPHAGAEGAEFLSPGEAAVDSYPNWLKFHIGINRYELYSRHNPAIEALLHDLSSQRITSVAMKSGGTQLKLIMTFQNYGQALFKPMKQTREQETPPDFFYFSDYERHNAEIAAFHLDRILDFRRVPPVAGRMVNMTKEIRDVTRDKKLWRTFFISPANNICFYGECSYYCSTEHALCGKPDQIEGSLAAFLPDLSLAKRKTWRNPWRRSYHKRKKAEWEVDPDYCEEVKQTPPYDSSHRILDVMDMTIFDFLMGNMDRHHYETFEKFGNETFIIHLDNGRGFGKYSHDELSILVPLQQCCRIRKSTYLRLQLLAKEEYKLSLLMAESLRGDQVAPVLYQPHLEALDRRLRVVLKAVRDCVERNGLHSVVDDDLDTEHRAASAR

SEQ ID NO: 349 - protein, 523 aa, synthetic construct
MVHIGFRSLKAVFILALSSLILYGIVTTFDGDFSSDPSSNLSSHSLEKLPPAAEPAERALRGRDPGALRPHDPAHRPLLRDPGPRRSESPPGPGGDASLLARLFEHPLYRVAVPPLTEEDVLFNVNSDTRLSPKAAENPDWPHAGAEGAEFLSPGEAAVDSYPNWLKFHIGINRYELYSRHNPAIEALLHDLSSQRITSVAMKSGGTQLKLIMTFQNYGQALFKPMKQTREQETPPDFFYFSDYERHNAEIAAFHLDRILDFRRVPPVAGRMVNMTKEIRDVTRDKKLWRTFFISPANNICFYGECSYYCSTEHALCGKPDQIEGSLAAFLPDLSLAKRKTWRNPWRRSYHKRKKAEWEVDPDYCEEVKQTPPYDSSHRILDVMDMTIFDFLMGNMDRHHYETFEKFGNETFIIHLDNGRGFGKYSHDELSILVPLQQCCRIRKSTYLRLQLLAKEEYKLSLLMAESLRGDQVAPVLYQPHLEALDRRLRVVLKAVRDCVERNGLHSVVDDDLDTEHRAASAR

SEQ ID NO: 350 - protein, 576 aa, synthetic construct
MVHIGFRSLKAVFILALSSLILYGIVTTFDGSRASRYQPPYVNHSQDPLYHSGNSYNRENATFVTLCRNEDLYSIIQSIKKVEDDFSSDPSSNLSSHSLEKLPPAAEPAERALRGRDPGALRPHDPAHRPLLRDPGPRRSESPPGPGGDASLLARLFEHPLYRVAVPPLTEEDVLFNVNSDTRLSPKAAENPDWPHAGAEGAEFLSPGEAAVDSYPNWLKFHIGINRYELYSRHNPAIEALLHDLSSQRITSVAMKSGGTQLKLIMTFQNYGQALFKPMKQTREQETPPDFFYFSDYERHNAEIAAFHLDRILDFRRVPPVAGRMVNMTKEIRDVTRDKKLWRTFFISPANNICFYGECSYYCSTEHALCGKPDQIEGSLAAFLPDLSLAKRKTWRNPWRRSYHKRKKAEWEVDPDYCEEVKQTPPYDSSHRILDVMDMTIFDFLMGNMDRHHYETFEKFGNETFIIHLDNGRGFGKYSHDELSILVPLQQCCRIRKSTYLRLQLLAKEEYKLSLLMAESLRGDQVAPVLYQPHLEALDRRLRVVLKAVRDCVERNGLHSVVDDDLDTEHRAASAR

SEQ ID NO: 351 - protein, 642 aa, synthetic construct
MVHIGFRSLKAVFILALSSLILYGIVTTFDGSRASRYQPPYVNHSQDPLYHSGNSYNRENATFVTLCRNEDLYSIIQSIKKVEDRFNNKFAYDWVFLNEVPFTDEFKERTSVLISGQAKYGLIPKEHWSYPDYIDQERAAESRRQLEDQHDFSSDPSSNLSSHSLEKLPPAAEPAERALRGRDPGALRPHDPAHRPLLRDPGPRRSESPPGPGGDASLLARLFEHPLYRVAVPPLTEEDVLFNVNSDTRLSPKAAENPDWPHAGAEGAEFLSPGEAAVDSYPNWLKFHIGINRYELYSRHNPAIEALLHDLSSQRITSVAMKSGGTQLKLIMTFQNYGQALFKPMKQTREQETPPDFFYFSDYERHNAEIAAFHLDRILDFRRVPPVAGRMVNMTKEIRDVTRDKKLWRTFFISPANNICFYGECSYYCSTEHALCGKPDQIEGSLAAFLPDLSLAKRKTWRNPWRRSYHKRKKAEWEVDPDYCEEVKQTPPYDSSHRILDVMDMTIFDFLMGNMDRHHYETFEKFGNETFIIHLDNGRGFGKYSHDELSILVPLQQCCRIRKSTYLRLQLLAKEEYKLSLLMAESLRGDQVAPVLYQPHLEALDRRLRVVLKAVRDCVERNGLHSVVDDDLDTEHRAASAR

SEQ ID NO: 352 - protein, 528 aa, synthetic construct
MLLTKRFSKLFKLTFIVLILCGLFVITNKYMDENTSDFSSDPSSNLSSHSLEKLPPAAEPAERALRGRDPGALRPHDPAHRPLLRDPGPRRSESPPGPGGDASLLARLFEHPLYRVAVPPLTEEDVLFNVNSDTRLSPKAAENPDWPHAGAEGAEFLSPGEAAVDSYPNWLKFHIGINRYELYSRHNPAIEALLHDLSSQRITSVAMKSGGTQLKLIMTFQNYGQALFKPMKQTREQETPPDFFYFSDYERHNAEIAAFHLDRILDFRRVPPVAGRMVNMTKEIRDVTRDKKLWRTFFISPANNICFYGECSYYCSTEHALCGKPDQIEGSLAAFLPDLSLAKRKTWRNPWRRSYHKRKKAEWEVDPDYCEEVKQTPPYDSSHRILDVMDMTIFDFLMGNMDRHHYETFEKFGNETFIIHLDNGRGFGKYSHDELSILVPLQQCCRIRKSTYLRLQLLAKEEYKLSLLMAESLRGDQVAPVLYQPHLEALDRRLRVVLKAVRDCVERNGLHSVVDDDLDTEHRAASAR

SEQ ID NO: 353 - protein, 589 aa, synthetic construct
MLLTKRFSKLFKLTFIVLILCGLFVITNKYMDENTSVKEYKEYLDRYVQSYSNKYSSSSDAASADDSTPLRDNDEAGNEKLKSFYNNVFNFLMVDSPDFSSDPSSNLSSHSLEKLPPAAEPAERALRGRDPGALRPHDPAHRPLLRDPGPRRSESPPGPGGDASLLARLFEHPLYRVAVPPLTEEDVLFNVNSDTRLSPKAAENPDWPHAGAEGAEFLSPGEAAVDSYPNWLKFHIGINRYELYSRHNPAIEALLHDLSSQRITSVAMKSGGTQLKLIMTFQNYGQALFKPMKQTREQETPPDFFYFSDYERHNAEIAAFHLDRILDFRRVPPVAGRMVNMTKEIRDVTRDKKLWRTFFISPANNICFYGECSYYCSTEHALCGKPDQIEGSLAAFLPDLSLAKRKTWRNPWRRSYHKRKKAEWEVDPDYCEEVKQTPPYDSSHRILDVMDMTIFDFLMGNMDRHHYETFEKFGNETFIIHLDNGRGFGKYSHDELSILVPLQQCCRIRKSTYLRLQLLAKEEYKLSLLMAESLRGDQVAPVLYQPHLEALDRRLRVVLKAVRDCVERNGLHSVVDDDLDTEHRAASAR

SEQ ID NO: 354 - protein, 642 aa, synthetic construct
MLLTKRFSKLFKLTFIVLILCGLFVITNKYMDENTSVKEYKEYLDRYVQSYSNKYSSSSDAASADDSTPLRDNDEAGNEKLKSFYNNVFNFLMVDSPKGSTAKQYNEACLLKGDIGDRPDHYKDLYKLSAKELSKCLELSPDEVASLTKSDFSSDPSSNLSSHSLEKLPPAAEPAERALRGRDPGALRPHDPAHRPLLRDPGPRRSESPPGPGGDASLLARLFEHPLYRVAVPPLTEEDVLFNVNSDTRLSPKAAENPDWPHAGAEGAEFLSPGEAAVDSYPNWLKFHIGINRYELYSRHNPAIEALLHDLSSQRITSVAMKSGGTQLKLIMTFQNYGQALFKPMKQTREQETPPDFFYFSDYERHNAEIAAFHLDRILDFRRVPPVAGRMVNMTKEIRDVTRDKKLWRTFFISPANNICFYGECSYYCSTEHALCGKPDQIEGSLAAFLPDLSLAKRKTWRNPWRRSYHKRKKAEWEVDPDYCEEVKQTPPYDSSHRILDVMDMTIFDFLMGNMDRHHYETFEKFGNETFIIHLDNGRGFGKYSHDELSILVPLQQCCRIRKSTYLRLQLLAKEEYKLSLLMAESLRGDQVAPVLYQPHLEALDRRLRVVLKAVRDCVERNGLHSVVDDDLDTEHRAASAR

SEQ ID NO: 355 - protein, 534 aa, synthetic construct
MLALRRFILNQRSLRSCTIPILVGALIIILVLFQLVTHRNDADFSSDPSSNLSSHSLEKLPPAAEPAERALRGRDPGALRPHDPAHRPLLRDPGPRRSESPPGPGGDASLLARLFEHPLYRVAVPPLTEEDVLFNVNSDTRLSPKAAENPDWPHAGAEGAEFLSPGEAAVDSYPNWLKFHIGINRYELYSRHNPAIEALLHDLSSQRITSVAMKSGGTQLKLIMTFQNYGQALFKPMKQTREQETPPDFFYFSDYERHNAEIAAFHLDRILDFRRVPPVAGRMVNMTKEIRDVTRDKKLWRTFFISPANNICFYGECSYYCSTEHALCGKPDQIEGSLAAFLPDLSLAKRKTWRNPWRRSYHKRKKAEWEVDPDYCEEVKQTPPYDSSHRILDVMDMTIFDFLMGNMDRHHYETFEKFGNETFIIHLDNGRGFGKYSHDELSILVPLQQCCRIRKSTYLRLQLLAKEEYKLSLLMAESLRGDQVAPVLYQPHLEALDRRLRVVLKAVRDCVERNGLHSVVDDDLDTEHRAASAR

SEQ ID NO: 356 - protein, 585 aa, synthetic construct
MLALRRFILNQRSLRSCTIPILVGALIIILVLFQLVTHRNDALIRSSNVNSTNKKTLKDADPKVLIEAFGSPEVDPVDTIPVSPLELVPFYDQDFSSDPSSNLSSHSLEKLPPAAEPAERALRGRDPGALRPHDPAHRPLLRDPGPRRSESPPGPGGDASLLARLFEHPLYRVAVPPLTEEDVLFNVNSDTRLSPKAAENPDWPHAGAEGAEFLSPGEAAVDSYPNWLKFHIGINRYELYSRHNPAIEALLHDLSSQRITSVAMKSGGTQLKLIMTFQNYGQALFKPMKQTREQETPPDFFYFSDYERHNAEIAAFHLDRILDFRRVPPVAGRMVNMTKEIRDVTRDKKLWRTFFISPANNICFYGECSYYCSTEHALCGKPDQIEGSLAAFLPDLSLAKRKTWRNPWRRSYHKRKKAEWEVDPDYCEEVKQTPPYDSSHRILDVMDMTIFDFLMGNMDRHHYETFEKFGNETFIIHLDNGRGFGKYSHDELSILVPLQQCCRIRKSTYLRLQLLAKEEYKLSLLMAESLRGDQVAPVLYQPHLEALDRRLRVVLKAVRDCVERNGLHSVVDDDLDTEHRAASAR

SEQ ID NO: 357 - protein, 645 aa, synthetic construct
MLALRRFILNQRSLRSCTIPILVGALIIILVLFQLVTHRNDALIRSSNVNSTNKKTLKDADPKVLIEAFGSPEVDPVDTIPVSPLELVPFYDQSIDTKRSSSWLINKKGYYKHFNELSLTDRCKFYFRTLYTLDDEWTNSVKKLEYSINDNEGDFSSDPSSNLSSHSLEKLPPAAEPAERALRGRDPGALRPHDPAHRPLLRDPGPRRSESPPGPGGDASLLARLFEHPLYRVAVPPLTEEDVLFNVNSDTRLSPKAAENPDWPHAGAEGAEFLSPGEAAVDSYPNWLKFHIGINRYELYSRHNPAIEALLHDLSSQRITSVAMKSGGTQLKLIMTFQNYGQALFKPMKQTREQETPPDFFYFSDYERHNAEIAAFHLDRILDFRRVPPVAGRMVNMTKEIRDVTRDKKLWRTFFISPANNICFYGECSYYCSTEHALCGKPDQIEGSLAAFLPDLSLAKRKTWRNPWRRSYHKRKKAEWEVDPDYCEEVKQTPPYDSSHRILDVMDMTIFDFLMGNMDRHHYETFEKFGNETFIIHLDNGRGFGKYSHDELSILVPLQQCCRIRKSTYLRLQLLAKEEYKLSLLMAESLRGDQVAPVLYQPHLEALDRRLRVVLKAVRDCVERNGLHSVVDDDLDTEHRAASAR

SEQ ID NO: 358 - protein, 522 aa, synthetic construct
MHVLLSKKIARFLLISFVFVLALMVTINHPDFSSDPSSNLSSHSLEKLPPAAEPAERALRGRDPGALRPHDPAHRPLLRDPGPRRSESPPGPGGDASLLARLFEHPLYRVAVPPLTEEDVLFNVNSDTRLSPKAAENPDWPHAGAEGAEFLSPGEAAVDSYPNWLKFHIGINRYELYSRHNPAIEALLHDLSSQRITSVAMKSGGTQLKLIMTFQNYGQALFKPMKQTREQETPPDFFYFSDYERHNAEIAAFHLDRILDFRRVPPVAGRMVNMTKEIRDVTRDKKLWRTFFISPANNICFYGECSYYCSTEHALCGKPDQIEGSLAAFLPDLSLAKRKTWRNPWRRSYHKRKKAEWEVDPDYCEEVKQTPPYDSSHRILDVMDMTIFDFLMGNMDRHHYETFEKFGNETFIIHLDNGRGFGKYSHDELSILVPLQQCCRIRKSTYLRLQLLAKEEYKLSLLMAESLRGDQVAPVLYQPHLEALDRRLRVVLKAVRDCVERNGLHSVVDDDLDTEHRAASAR

SEQ ID NO: 359 - protein, 577 aa, synthetic construct
MHVLLSKKIARFLLISFVFVLALMVTINHPKTKQMSEQYVTPYLPKSLQPIAKISAEEQRRIQSEQEEAELKQSLEGEAIRNATVDFSSDPSSNLSSHSLEKLPPAAEPAERALRGRDPGALRPHDPAHRPLLRDPGPRRSESPPGPGGDASLLARLFEHPLYRVAVPPLTEEDVLFNVNSDTRLSPKAAENPDWPHAGAEGAEFLSPGEAAVDSYPNWLKFHIGINRYELYSRHNPAIEALLHDLSSQRITSVAMKSGGTQLKLIMTFQNYGQALFKPMKQTREQETPPDFFYFSDYERHNAEIAAFHLDRILDFRRVPPVAGRMVNMTKEIRDVTRDKKLWRTFFISPANNICFYGECSYYCSTEHALCGKPDQIEGSLAAFLPDLSLAKRKTWRNPWRRSYHKRKKAEWEVDPDYCEEVKQTPPYDSSHRILDVMDMTIFDFLMGNMDRHHYETFEKFGNETFIIHLDNGRGFGKYSHDELSILVPLQQCCRIRKSTYLRLQLLAKEEYKLSLLMAESLRGDQVAPVLYQPHLEALDRRLRVVLKAVRDCVERNGLHSVVDDDLDTEHRAASAR

SEQ ID NO: 360 - protein, 652 aa, synthetic construct
MHVLLSKKIARFLLISFVFVLALMVTINHPKTKQMSEQYVTPYLPKSLQPIAKISAEEQRRIQSEQEEAELKQSLEGEAIRNATVNAIKEKIKSYGGNETTLGFMVPSYINHRGSPPKACFVSLITERDSMTQILQSIDEVQVKFNKNFAYPWVFISQGEDFSSDPSSNLSSHSLEKLPPAAEPAERALRGRDPGALRPHDPAHRPLLRDPGPRRSESPPGPGGDASLLARLFEHPLYRVAVPPLTEEDVLFNVNSDTRLSPKAAENPDWPHAGAEGAEFLSPGEAAVDSYPNWLKFHIGINRYELYSRHNPAIEALLHDLSSQRITSVAMKSGGTQLKLIMTFQNYGQALFKPMKQTREQETPPDFFYFSDYERHNAEIAAFHLDRILDFRRVPPVAGRMVNMTKEIRDVTRDKKLWRTFFISPANNICFYGECSYYCSTEHALCGKPDQIEGSLAAFLPDLSLAKRKTWRNPWRRSYHKRKKAEWEVDPDYCEEVKQTPPYDSSHRILDVMDMTIFDFLMGNMDRHHYETFEKFGNETFIIHLDNGRGFGKYSHDELSILVPLQQCCRIRKSTYLRLQLLAKEEYKLSLLMAESLRGDQVAPVLYQPHLEALDRRLRVVLKAVRDCVERNGLHSVVDDDLDTEHRAASAR

SEQ ID NO: 361 - DNA, 1779 bp, synthetic construct
  source 1..1779: mol_type = other DNA
atgaaaatgatgttggttagaagatttagagttttgattttgatggtttttttggttgcttgtgctttgcatattgctttggatttgttgccaagattggaaagaagaggtgctagaccatctggtgaacctggttgttcttgtgctcaacctgctgctgaagtcgctgctcctggttgggctcaagttagaggtagacctggtgaaccacctgctgcttcgtcagctgctggtgatgctggttggccaaataaacatactttgagaattttgcaagatttttcttctgatccatcttctaatttatctagtcactctttggaaaaattgccacctgctgctgagcctgctgaaagagctttgagaggtagagaccctggtgcgctcagaccacatgatcctgctcatagaccattgttgagagaccctggtcctagaagatctgaatctccacctggtcctggtggtgatgcttctttgttggctagattgtttgaacatccattgtatagagttgctgttccaccattgactgaagaagatgttttgtttaatgttaattctgatactagattgtctccaaaagctgctgaaaatcctgattggccacatgctggtgctgaaggtgctgaatttttgtctcctggtgaagcggctgttgattcctatccaaattggttgaaattccacatcggtattaaccgatacgaattgtattctagacataatcctgctattgaagctttgttgcatgatttgtctagtcagagaattacttctgttgctatgaagtctggtggtactcagttgaagttgataatgacttttcaaaactatgggcaagctttgtttaaaccaatgaaacaaacgagagaacaagaaactccacctgattttttttatttttcggattatgaaagacataatgctgaaattgctgctttccacttggacagaatattggattttcgcagagttccacctgttgctggtcggatggttaacatgactaaggaaattagagatgttactagagataaaaaattgtggagaacttttttcatttctccggctaataatatttgtttttacggggaatgttcttattattgttctactgaacatgctttgtgtggtaaacctgatcaaattgaaggttctttggctgcttttttgcctgatttgtctttggctaaaagaaaaacttggagaaatccatggagaagatcttatcataaaagaaaaaaagctgaatgggaagttgatcctgattattgtgaagaagttaaacaaactccaccatatgattcgtctcatagaatattggacgtcatggatatgacgatctttgactttctgatggggaacatggacagacatcactatgaaacattcgaaaaattcggtaatgaaacttttatcatccatttggataatggtagaggttttggtaaatattctcatgatgaattgtctattttggttccattgcaacagtgttgtagaataaggaaaagcacttacttaagattacaactcttggctaaagaagaatataaattgtctttgttgatggctgaatctttgagaggtgatcaagttgctcctgttttgtatcaaccacatttggaagctttggatagaagattgagagttgttttgaaagctgttagagattgtgttgaaagaaatggtttgcattctgttgttgatgatgatttggatactgaacatagagctgcttctgctagagattataaagatgatgatgataaataa

SEQ ID NO: 362 - DNA, 1689 bp, synthetic construct
  source 1..1689: mol_type = other DNA
atgagattggaaagaagaggtgctagaccatctggtgaacctggttgttcttgtgctcaacctgctgctgaagtcgctgctcctggttgggctcaagttagaggtagacctggtgaaccacctgctgcttcgtcagctgctggtgatgctggttggccaaataaacatactttgagaattttgcaagatttttcttctgatccatcttctaatttatctagtcactctttggaaaaattgccacctgctgctgagcctgctgaaagagctttgagaggtagagaccctggtgcgctcagaccacatgatcctgctcatagaccattgttgagagaccctggtcctagaagatctgaatctccacctggtcctggtggtgatgcttctttgttggctagattgtttgaacatccattgtatagagttgctgttccaccattgactgaagaagatgttttgtttaatgttaattctgatactagattgtctccaaaagctgctgaaaatcctgattggccacatgctggtgctgaaggtgctgaatttttgtctcctggtgaagcggctgttgattcctatccaaattggttgaaattccacatcggtattaaccgatacgaattgtattctagacataatcctgctattgaagctttgttgcatgatttgtctagtcagagaattacttctgttgctatgaagtctggtggtactcagttgaagttgataatgacttttcaaaactatgggcaagctttgtttaaaccaatgaaacaaacgagagaacaagaaactccacctgattttttttatttttcggattatgaaagacataatgctgaaattgctgctttccacttggacagaatattggattttcgcagagttccacctgttgctggtcggatggttaacatgactaaggaaattagagatgttactagagataaaaaattgtggagaacttttttcatttctccggctaataatatttgtttttacggggaatgttcttattattgttctactgaacatgctttgtgtggtaaacctgatcaaattgaaggttctttggctgcttttttgcctgatttgtctttggctaaaagaaaaacttggagaaatccatggagaagatcttatcataaaagaaaaaaagctgaatgggaagttgatcctgattattgtgaagaagttaaacaaactccaccatatgattcgtctcatagaatattggacgtcatggatatgacgatctttgactttctgatggggaacatggacagacatcactatgaaacattcgaaaaattcggtaatgaaacttttatcatccatttggataatggtagaggttttggtaaatattctcatgatgaattgtctattttggttccattgcaacagtgttgtagaataaggaaaagcacttacttaagattacaactcttggctaaagaagaatataaattgtctttgttgatggctgaatctttgagaggtgatcaagttgctcctgttttgtatcaaccacatttggaagctttggatagaagattgagagttgttttgaaagctgttagagattgtgttgaaagaaatggtttgcattctgttgttgatgatgatttggatactgaacatagagctgcttctgctagagattataaagatgatgatgataaataa

SEQ ID NO: 363 - DNA, 1590 bp, synthetic construct
  source 1..1590: mol_type = other DNA
agaggtagacctggtgaaccaccggctgcgtcctctgctgctggtgatgctggttggccaaataaacatactttgagaattttgcaagatttttcttctgatccatcttctaatctatctagtcactcattggaaaaattgccacctgctgcagagcctgctgaaagagctttgagaggtagagatcccggtgcgttaagaccacatgatcctgctcatagaccattgttgagagatcctggtccaagaagatctgaatctccacctggtcctggtggtgatgcttctttgttggctagattgtttgaacatccattgtatagagttgctgttccaccattgactgaagaagatgttttgtttaatgttaattctgatactagattgtctccaaaagctgctgaaaatcctgattggccacatgctggtgctgaaggtgctgaatttttgtctcctggtgaagcagctgttgattcttatcccaattggttgaaattccatattggtattaacagatacgaattgtattctagacataatcctgctattgaagctttgttgcatgatctttcttctcaacgtattacttctgttgctatgaaatcgggtggtactcaattgaaattgattatgacttttcaaaactatggtcaagctttgtttaaaccaatgaaacagactagagagcaagaaacgccacctgattttttttacttctctgattatgaaagacataatgctgaaattgctgcttttcacttggaccgtatattggattttcgcagagttccacctgttgctggtcgtatggttaacatgactaaggaaattagagatgttactcgcgacaaaaaattgtggagaactttttttatatctccggccaataatatttgtttttatggtgaatgttcttattattgttctactgaacatgctttgtgtggtaaacctgatcaaattgaaggttctttggctgcttttttgcctgatttgtctttggctaaaagaaaaacttggagaaatccatggagaagatcttatcataaaagaaaaaaagctgaatgggaagttgatcctgattattgtgaagaagttaaacaaactccaccatatgattcttctcacagaattttggatgttatggatatgactattttcgattttttgatgggtaacatggatagacatcattatgaaacgtttgagaaatttgggaatgaaacttttattattcatttggacaacggtagaggttttggtaaatattctcatgatgaattgtctattttggttccattgcagcaatgttgcagaataagaaaatcgacttatttgcgactgcagcttttggctaaagaagaatataaattgtctttgttgatggctgaatctttgagaggtgatcaagttgctcctgttttgtatcaaccacatttggaagctttggatagaagattgagagttgttttgaaagctgttagagattgtgttgaaagaaatggtttgcattctgttgttgatgatgatttggatactgaacatagagctgcttctgctagagattataaagatgatgatgataaataa

SEQ ID NO: 364 - DNA, 1503 bp, synthetic construct
  source 1..1503: mol_type = other DNA
gatttttcttctgatccatcttctaatctatctagtcactcattggaaaaattgccacctgctgcagagcctgctgaaagagctttgagaggtagagatcccggtgcgttaagaccacatgatcctgctcatagaccattgttgagagatcctggtccaagaagatctgaatctccacctggtcctggtggtgatgcttctttgttggctagattgtttgaacatccattgtatagagttgctgttccaccattgactgaagaagatgttttgtttaatgttaattctgatactagattgtctccaaaagctgctgaaaatcctgattggccacatgctggtgctgaaggtgctgaatttttgtctcctggtgaagcagctgttgattcttatcccaattggttgaaattccatattggtattaacagatacgaattgtattctagacataatcctgctattgaagctttgttgcatgatctttcttctcaacgtattacttctgttgctatgaaatcgggtggtactcaattgaaattgattatgacttttcaaaactatggtcaagctttgtttaaaccaatgaaacagactagagagcaagaaacgccacctgattttttttacttctctgattatgaaagacataatgctgaaattgctgcttttcacttggaccgtatattggattttcgcagagttccacctgttgctggtcgtatggttaacatgactaaggaaattagagatgttactcgcgacaaaaaattgtggagaactttttttatatctccggccaataatatttgtttttatggtgaatgttcttattattgttctactgaacatgctttgtgtggtaaacctgatcaaattgaaggttctttggctgcttttttgcctgatttgtctttggctaaaagaaaaacttggagaaatccatggagaagatcttatcataaaagaaaaaaagctgaatgggaagttgatcctgattattgtgaagaagttaaacaaactccaccatatgattcttctcacagaattttggatgttatggatatgactattttcgattttttgatgggtaacatggatagacatcattatgaaacgtttgagaaatttgggaatgaaacttttattattcatttggacaacggtagaggttttggtaaatattctcatgatgaattgtctattttggttccattgcagcaatgttgcagaataagaaaatcgacttatttgcgactgcagcttttggctaaagaagaatataaattgtctttgttgatggctgaatctttgagaggtgatcaagttgctcctgttttgtatcaaccacatttggaagctttggatagaagattgagagttgttttgaaagctgttagagattgtgttgaaagaaatggtttgcattctgttgttgatgatgatttggatactgaacatagagctgcttctgctagagattataaagatgatgatgataaataa

SEQ ID NO: 365 - DNA, 915 bp, synthetic construct
  source 1..915: mol_type = other DNA
cagactagagagcaagaaacgccacctgattttttttacttctctgattatgaaagacataatgctgaaattgctgcttttcacttggaccgtatattggattttcgcagagttccacctgttgctggtcgtatggttaacatgactaaggaaattagagatgttactcgcgacaaaaaattgtggagaactttttttatatctccggccaataatatttgtttttatggtgaatgttcttattattgttctactgaacatgctttgtgtggtaaacctgatcaaattgaaggttctttggctgcttttttgcctgatttgtctttggctaaaagaaaaacttggagaaatccatggagaagatcttatcataaaagaaaaaaagctgaatgggaagttgatcctgattattgtgaagaagttaaacaaactccaccatatgattcttctcacagaattttggatgttatggatatgactattttcgattttttgatgggtaacatggatagacatcattatgaaacgtttgagaaatttgggaatgaaacttttattattcatttggacaacggtagaggttttggtaaatattctcatgatgaattgtctattttggttccattgcagcaatgttgcagaataagaaaatcgacttatttgcgactgcagcttttggctaaagaagaatataaattgtctttgttgatggctgaatctttgagaggtgatcaagttgctcctgttttgtatcaaccacatttggaagctttggatagaagattgagagttgttttgaaagctgttagagattgtgttgaaagaaatggtttgcattctgttgttgatgatgatttggatactgaacatagagctgcttctgctagagattataaagatgatgatgataaataa

SEQ ID NO: 366 - DNA, 1788 bp, synthetic construct
  source 1..1788: mol_type = other DNA
atgaaaatgatgttggttagaagatttagagttttgattttgatggtttttttggttgcttgtgctttgcatattgctttggatttgttgccaagattggaaagaagaggtgctagaccatctggtgaacctggttgttcttgtgctcaacctgctgctgaagtcgctgctcctggttgggctcaagttagaggtagacctggtgaaccacctgctgcttcgtcagctgctggtgatgctggttggccaaataaacatactttgagaattttgcaagatttttcttctgatccatcttctaatttatctagtcactctttggaaaaattgccacctgctgctgagcctgctgaaagagctttgagaggtagagaccctggtgcgctcagaccacatgatcctgctcatagaccattgttgagagaccctggtcctagaagatctgaatctccacctggtcctggtggtgatgcttctttgttggctagattgtttgaacatccattgtatagagttgctgttccaccattgactgaagaagatgttttgtttaatgttaattctgatactagattgtctccaaaagctgctgaaaatcctgattggccacatgctggtgctgaaggtgctgaatttttgtctcctggtgaagcggctgttgattcctatccaaattggttgaaattccacatcggtattaaccgatacgaattgtattctagacataatcctgctattgaagctttgttgcatgatttgtctagtcagagaattacttctgttgctatgaagtctggtggtactcagttgaagttgataatgacttttcaaaactatgggcaagctttgtttaaaccaatgaaacaaacgagagaacaagaaactccacctgattttttttatttttcggattatgaaagacataatgctgaaattgctgctttccacttggacagaatattggattttcgcagagttccacctgttgctggtcggatggttaacatgactaaggaaattagagatgttactagagataaaaaattgtggagaacttttttcatttctccggctaataatatttgtttttacggggaatgttcttattattgttctactgaacatgctttgtgtggtaaacctgatcaaattgaaggttctttggctgcttttttgcctgatttgtctttggctaaaagaaaaacttggagaaatccatggagaagatcttatcataaaagaaaaaaagctgaatgggaagttgatcctgattattgtgaagaagttaaacaaactccaccatatgattcgtctcatagaatattggacgtcatggatatgacgatctttgactttctgatggggaacatggacagacatcactatgaaacattcgaaaaattcggtaatgaaacttttatcatccatttggataatggtagaggttttggtaaatattctcatgatgaattgtctattttggttccattgcaacagtgttgtagaataaggaaaagcacttacttaagattacaactcttggctaaagaagaatataaattgtctttgttgatggctgaatctttgagaggtgatcaagttgctcctgttttgtatcaaccacatttggaagctttggatagaagattgagagttgttttgaaagctgttagagattgtgttgaaagaaatggtttgcattctgttgttgatgatgatttggatactgaacatagagctgcttctgctagagaacagaagctgatcagtgaagaagatctgtgatga

SEQ ID NO: 367 - DNA, 1689 bp, synthetic construct
  source 1..1689: mol_type = other DNA
atgagattggaaagaagaggtgctagaccatctggtgaacctggttgttcttgtgctcaacctgctgctgaagtcgctgctcctggttgggctcaagttagaggtagacctggtgaaccacctgctgcttcgtcagctgctggtgatgctggttggccaaataaacatactttgagaattttgcaagatttttcttctgatccatcttctaatttatctagtcactctttggaaaaattgccacctgctgctgagcctgctgaaagagctttgagaggtagagaccctggtgcgctcagaccacatgatcctgctcatagaccattgttgagagaccctggtcctagaagatctgaatctccacctggtcctggtggtgatgcttctttgttggctagattgtttgaacatccattgtatagagttgctgttccaccattgactgaagaagatgttttgtttaatgttaattctgatactagattgtctccaaaagctgctgaaaatcctgattggccacatgctggtgctgaaggtgctgaatttttgtctcctggtgaagcggctgttgattcctatccaaattggttgaaattccacatcggtattaaccgatacgaattgtattctagacataatcctgctattgaagctttgttgcatgatttgtctagtcagagaattacttctgttgctatgaagtctggtggtactcagttgaagttgataatgacttttcaaaactatgggcaagctttgtttaaaccaatgaaacaaacgagagaacaagaaactccacctgattttttttatttttcggattatgaaagacataatgctgaaattgctgctttccacttggacagaatattggattttcgcagagttccacctgttgctggtcggatggttaacatgactaaggaaattagagatgttactagagataaaaaattgtggagaacttttttcatttctccggctaataatatttgtttttacggggaatgttcttattattgttctactgaacatgctttgtgtggtaaacctgatcaaattgaaggttctttggctgcttttttgcctgatttgtctttggctaaaagaaaaacttggagaaatccatggagaagatcttatcataaaagaaaaaaagctgaatgggaagttgatcctgattattgtgaagaagttaaacaaactccaccatatgattcgtctcatagaatattggacgtcatggatatgacgatctttgactttctgatggggaacatggacagacatcactatgaaacattcgaaaaattcggtaatgaaacttttatcatccatttggataatggtagaggttttggtaaatattctcatgatgaattgtctattttggttccattgcaacagtgttgtagaataaggaaaagcacttacttaagattacaactcttggctaaagaagaatataaattgtctttgttgatggctgaatctttgagaggtgatcaagttgctcctgttttgtatcaaccacatttggaagctttggatagaagattgagagttgttttgaaagctgttagagattgtgttgaaagaaatggtttgcattctgttgttgatgatgatttggatactgaacatagagctgcttctgctagagattataaagatgatgatgataaatga

SEQ ID NO: 368 - DNA, 1860 bp, synthetic construct
  source 1..1860: mol_type = other DNA
atggctttatttttgtctaaaagattgttgagatttactgttattgctggtgctgttattgttctattgttgacgctcaattcaaattcacgaactcaacaatatattccatcttctatttctgctgcttttgattttacttctggttctatttctcctgaacaacaagttattagattggaaagaagaggtgctagaccatctggtgaacctggttgttcttgtgctcaacctgctgctgaagtcgctgctcctggttgggctcaagttagaggtagacctggtgaaccacctgctgcttcgtcagctgctggtgatgctggttggccaaataaacatactttgagaattttgcaagatttttcttctgatccatcttctaatttatctagtcactctttggaaaaattgccacctgctgctgagcctgctgaaagagctttgagaggtagagaccctggtgcgctcagaccacatgatcctgctcatagaccattgttgagagaccctggtcctagaagatctgaatctccacctggtcctggtggtgatgcttctttgttggctagattgtttgaacatccattgtatagagttgctgttccaccattgactgaagaagatgttttgtttaatgttaattctgatactagattgtctccaaaagctgctgaaaatcctgattggccacatgctggtgctgaaggtgctgaatttttgtctcctggtgaagcggctgttgattcctatccaaattggttgaaattccacatcggtattaaccgatacgaattgtattctagacataatcctgctattgaagctttgttgcatgatttgtctagtcagagaattacttctgttgctatgaagtctggtggtactcagttgaagttgataatgacttttcaaaactatgggcaagctttgtttaaaccaatgaaacaaacgagagaacaagaaactccacctgattttttttatttttcggattatgaaagacataatgctgaaattgctgctttccacttggacagaatattggattttcgcagagttccacctgttgctggtcggatggttaacatgactaaggaaattagagatgttactagagataaaaaattgtggagaacttttttcatttctccggctaataatatttgtttttacggggaatgttcttattattgttctactgaacatgctttgtgtggtaaacctgatcaaattgaaggttctttggctgcttttttgcctgatttgtctttggctaaaagaaaaacttggagaaatccatggagaagatcttatcataaaagaaaaaaagctgaatgggaagttgatcctgattattgtgaagaagttaaacaaactccaccatatgattcgtctcatagaatattggacgtcatggatatgacgatctttgactttctgatggggaacatggacagacatcactatgaaacattcgaaaaattcggtaatgaaacttttatcatccatttggataatggtagaggttttggtaaatattctcatgatgaattgtctattttggttccattgcaacagtgttgtagaataaggaaaagcacttacttaagattacaactcttggctaaagaagaatataaattgtctttgttgatggctgaatctttgagaggtgatcaagttgctcctgttttgtatcaaccacatttggaagctttggatagaagattgagagttgttttgaaagctgttagagattgtgttgaaagaaatggtttgcattctgttgttgatgatgatttggatactgaacatagagctgcttctgctagagattataaagatgatgatgataaatga

SEQ ID NO: 369 - DNA, 1926 bp, synthetic construct
  source 1..1926: mol_type = other DNA
atggctttatttttgtctaaaagattgttgagatttactgttattgctggtgctgttattgttctattgttgacgctcaattcaaattcacgaactcaacaatatattccatcttctatttctgctgcttttgattttacttctggttctatttctcctgaacaacaagttatttctgaagaaaatgatgctaagaaattagaacagtccgctttgaattctgaagcttctgaagattctagattggaaagaagaggtgctagaccatctggtgaacctggttgttcttgtgctcaacctgctgctgaagtcgctgctcctggttgggctcaagttagaggtagacctggtgaaccacctgctgcttcgtcagctgctggtgatgctggttggccaaataaacatactttgagaattttgcaagatttttcttctgatccatcttctaatttatctagtcactctttggaaaaattgccacctgctgctgagcctgctgaaagagctttgagaggtagagaccctggtgcgctcagaccacatgatcctgctcatagaccattgttgagagaccctggtcctagaagatctgaatctccacctggtcctggtggtgatgcttctttgttggctagattgtttgaacatccattgtatagagttgctgttccaccattgactgaagaagatgttttgtttaatgttaattctgatactagattgtctccaaaagctgctgaaaatcctgattggccacatgctggtgctgaaggtgctgaatttttgtctcctggtgaagcggctgttgattcctatccaaattggttgaaattccacatcggtattaaccgatacgaattgtattctagacataatcctgctattgaagctttgttgcatgatttgtctagtcagagaattacttctgttgctatgaagtctggtggtactcagttgaagttgataatgacttttcaaaactatgggcaagctttgtttaaaccaatgaaacaaacgagagaacaagaaactccacctgattttttttatttttcggattatgaaagacataatgctgaaattgctgctttccacttggacagaatattggattttcgcagagttccacctgttgctggtcggatggttaacatgactaaggaaattagagatgttactagagataaaaaattgtggagaacttttttcatttctccggctaataatatttgtttttacggggaatgttcttattattgttctactgaacatgctttgtgtggtaaacctgatcaaattgaaggttctttggctgcttttttgcctgatttgtctttggctaaaagaaaaacttggagaaatccatggagaagatcttatcataaaagaaaaaaagctgaatgggaagttgatcctgattattgtgaagaagttaaacaaactccaccatatgattcgtctcatagaatattggacgtcatggatatgacgatctttgactttctgatggggaacatggacagacatcactatgaaacattcgaaaaattcggtaatgaaacttttatcatccatttggataatggtagaggttttggtaaatattctcatgatgaattgtctattttggttccattgcaacagtgttgtagaataaggaaaagcacttacttaagattacaactcttggctaaagaagaatataaattgtctttgttgatggctgaatctttgagaggtgatcaagttgctcctgttttgtatcaaccacatttggaagctttggatagaagattgagagttgttttgaaagctgttagagattgtgttgaaagaaatggtttgcattctgttgttgatgatgatttggatactgaacatagagctgcttctgctagagattataaagatgatgatgataaatga

SEQ ID NO: 370 - DNA, 1992 bp, synthetic construct
  source 1..1992: mol_type = other DNA
atggctttatttttgtctaaaagattgttgagatttactgttattgctggtgctgttattgttctattgttgacgctcaattcaaattcacgaactcaacaatatattccatcttctatttctgctgcttttgattttacttctggttctatttctcctgaacaacaagttatttctgaagaaaatgatgctaagaaattagaacagtccgctttgaattctgaagcttctgaagattctgaagctatggatgaagaatctaaagctttgaaagctgctgctgaaaaagctgatgctccaattgatagattggaaagaagaggtgctagaccatctggtgaacctggttgttcttgtgctcaacctgctgctgaagtcgctgctcctggttgggctcaagttagaggtagacctggtgaaccacctgctgcttcgtcagctgctggtgatgctggttggccaaataaacatactttgagaattttgcaagatttttcttctgatccatcttctaatttatctagtcactctttggaaaaattgccacctgctgctgagcctgctgaaagagctttgagaggtagagaccctggtgcgctcagaccacatgatcctgctcatagaccattgttgagagaccctggtcctagaagatctgaatctccacctggtcctggtggtgatgcttctttgttggctagattgtttgaacatccattgtatagagttgctgttccaccattgactgaagaagatgttttgtttaatgttaattctgatactagattgtctccaaaagctgctgaaaatcctgattggccacatgctggtgctgaaggtgctgaatttttgtctcctggtgaagcggctgttgattcctatccaaattggttgaaattccacatcggtattaaccgatacgaattgtattctagacataatcctgctattgaagctttgttgcatgatttgtctagtcagagaattacttctgttgctatgaagtctggtggtactcagttgaagttgataatgacttttcaaaactatgggcaagctttgtttaaaccaatgaaacaaacgagagaacaagaaactccacctgattttttttatttttcggattatgaaagacataatgctgaaattgctgctttccacttggacagaatattggattttcgcagagttccacctgttgctggtcggatggttaacatgactaaggaaattagagatgttactagagataaaaaattgtggagaacttttttcatttctccggctaataatatttgtttttacggggaatgttcttattattgttctactgaacatgctttgtgtggtaaacctgatcaaattgaaggttctttggctgcttttttgcctgatttgtctttggctaaaagaaaaacttggagaaatccatggagaagatcttatcataaaagaaaaaaagctgaatgggaagttgatcctgattattgtgaagaagttaaacaaactccaccatatgattcgtctcatagaatattggacgtcatggatatgacgatctttgactttctgatggggaacatggacagacatcactatgaaacattcgaaaaattcggtaatgaaacttttatcatccatttggataatggtagaggttttggtaaatattctcatgatgaattgtctattttggttccattgcaacagtgttgtagaataaggaaaagcacttacttaagattacaactcttggctaaagaagaatataaattgtctttgttgatggctgaatctttgagaggtgatcaagttgctcctgttttgtatcaaccacatttggaagctttggatagaagattgagagttgttttgaaagctgttagagattgtgttgaaagaaatggtttgcattctgttgttgatgatgatttggatactgaacatagagctgcttctgctagagattataaagatgatgatgataaatga

SEQ ID NO: 371 - DNA, 1779 bp, synthetic construct
  source 1..1779: mol_type = other DNA
atggttcatataggttttagaagcttgaaagccgttttcattttagcattgtcctcattgattttgtatggtattgttactacttttgatggtagattggaaagaagaggtgctagaccatctggtgaacctggttgttcttgtgctcaacctgctgctgaagtcgctgctcctggttgggctcaagttagaggtagacctggtgaaccacctgctgcttcgtcagctgctggtgatgctggttggccaaataaacatactttgagaattttgcaagatttttcttctgatccatcttctaatttatctagtcactctttggaaaaattgccacctgctgctgagcctgctgaaagagctttgagaggtagagaccctggtgcgctcagaccacatgatcctgctcatagaccattgttgagagaccctggtcctagaagatctgaatctccacctggtcctggtggtgatgcttctttgttggctagattgtttgaacatccattgtatagagttgctgttccaccattgactgaagaagatgttttgtttaatgttaattctgatactagattgtctccaaaagctgctgaaaatcctgattggccacatgctggtgctgaaggtgctgaatttttgtctcctggtgaagcggctgttgattcctatccaaattggttgaaattccacatcggtattaaccgatacgaattgtattctagacataatcctgctattgaagctttgttgcatgatttgtctagtcagagaattacttctgttgctatgaagtctggtggtactcagttgaagttgataatgacttttcaaaactatgggcaagctttgtttaaaccaatgaaacaaacgagagaacaagaaactccacctgattttttttatttttcggattatgaaagacataatgctgaaattgctgctttccacttggacagaatattggattttcgcagagttccacctgttgctggtcggatggttaacatgactaaggaaattagagatgttactagagataaaaaattgtggagaacttttttcatttctccggctaataatatttgtttttacggggaatgttcttattattgttctactgaacatgctttgtgtggtaaacctgatcaaattgaaggttctttggctgcttttttgcctgatttgtctttggctaaaagaaaaacttggagaaatccatggagaagatcttatcataaaagaaaaaaagctgaatgggaagttgatcctgattattgtgaagaagttaaacaaactccaccatatgattcgtctcatagaatattggacgtcatggatatgacgatctttgactttctgatggggaacatggacagacatcactatgaaacattcgaaaaattcggtaatgaaacttttatcatccatttggataatggtagaggttttggtaaatattctcatgatgaattgtctattttggttccattgcaacagtgttgtagaataaggaaaagcacttacttaagattacaactcttggctaaagaagaatataaattgtctttgttgatggctgaatctttgagaggtgatcaagttgctcctgttttgtatcaaccacatttggaagctttggatagaagattgagagttgttttgaaagctgttagagattgtgttgaaagaaatggtttgcattctgttgttgatgatgatttggatactgaacatagagctgcttctgctagagattataaagatgatgatgataaatga

SEQ ID NO: 372 - DNA, 1938 bp, synthetic construct
  source 1..1938: mol_type = other DNA
atggttcatataggttttagaagcttgaaagccgttttcattttagcattgtcctcattgattttgtatggtattgttactacttttgatggttctagagcttctagatatcaaccaccatatgttaatcattctcaagatccattgtaccattctggtaactcctataatagagaaaacgcgacttttgttaccttgtgtagaaatgaagatttgtattctattatccaatctatcaagaaagtcgaagacagattggaaagaagaggtgctagaccatctggtgaacctggttgttcttgtgctcaacctgctgctgaagtcgctgctcctggttgggctcaagttagaggtagacctggtgaaccacctgctgcttcgtcagctgctggtgatgctggttggccaaataaacatactttgagaattttgcaagatttttcttctgatccatcttctaatttatctagtcactctttggaaaaattgccacctgctgctgagcctgctgaaagagctttgagaggtagagaccctggtgcgctcagaccacatgatcctgctcatagaccattgttgagagaccctggtcctagaagatctgaatctccacctggtcctggtggtgatgcttctttgttggctagattgtttgaacatccattgtatagagttgctgttccaccattgactgaagaagatgttttgtttaatgttaattctgatactagattgtctccaaaagctgctgaaaatcctgattggccacatgctggtgctgaaggtgctgaatttttgtctcctggtgaagcggctgttgattcctatccaaattggttgaaattccacatcggtattaaccgatacgaattgtattctagacataatcctgctattgaagctttgttgcatgatttgtctagtcagagaattacttctgttgctatgaagtctggtggtactcagttgaagttgataatgacttttcaaaactatgggcaagctttgtttaaaccaatgaaacaaacgagagaacaagaaactccacctgattttttttatttttcggattatgaaagacataatgctgaaattgctgctttccacttggacagaatattggattttcgcagagttccacctgttgctggtcggatggttaacatgactaaggaaattagagatgttactagagataaaaaattgtggagaacttttttcatttctccggctaataatatttgtttttacggggaatgttcttattattgttctactgaacatgctttgtgtggtaaacctgatcaaattgaaggttctttggctgcttttttgcctgatttgtctttggctaaaagaaaaacttggagaaatccatggagaagatcttatcataaaagaaaaaaagctgaatgggaagttgatcctgattattgtgaagaagttaaacaaactccaccatatgattcgtctcatagaatattggacgtcatggatatgacgatctttgactttctgatggggaacatggacagacatcactatgaaacattcgaaaaattcggtaatgaaacttttatcatccatttggataatggtagaggttttggtaaatattctcatgatgaattgtctattttggttccattgcaacagtgttgtagaataaggaaaagcacttacttaagattacaactcttggctaaagaagaatataaattgtctttgttgatggctgaatctttgagaggtgatcaagttgctcctgttttgtatcaaccacatttggaagctttggatagaagattgagagttgttttgaaagctgttagagattgtgttgaaagaaatggtttgcattctgttgttgatgatgatttggatactgaacatagagctgcttctgctagagattataaagatgatgatgataaatga

SEQ ID NO: 373 - DNA, 2136 bp, synthetic construct
  source 1..2136: mol_type = other DNA
atggttcatataggttttagaagcttgaaagccgttttcattttagcattgtcctcattgattttgtatggtattgttactacttttgatggttctagagcttctagatatcaaccaccatatgttaatcattctcaagatccattgtaccattctggtaactcctataatagagaaaacgcgacttttgttaccttgtgtagaaatgaagatttgtattctattatccaatctatcaagaaagtcgaagaccgatttaacaacaaatttgcatacgattgggtttttctgaatgaagttccctttactgatgaatttaaagagaggacttctgttttgatttctggtcaagctaaatatggtttgattccaaaagaacattggtcttatcctgattatattgatcaagaaagagctgctgaatctagaagacaattggaagatcaacatagattggaaagaagaggtgctagaccatctggtgaacctggttgttcttgtgctcaacctgctgctgaagtcgctgctcctggttgggctcaagttagaggtagacctggtgaaccacctgctgcttcgtcagctgctggtgatgctggttggccaaataaacatactttgagaattttgcaagatttttcttctgatccatcttctaatttatctagtcactctttggaaaaattgccacctgctgctgagcctgctgaaagagctttgagaggtagagaccctggtgcgctcagaccacatgatcctgctcatagaccattgttgagagaccctggtcctagaagatctgaatctccacctggtcctggtggtgatgcttctttgttggctagattgtttgaacatccattgtatagagttgctgttccaccattgactgaagaagatgttttgtttaatgttaattctgatactagattgtctccaaaagctgctgaaaatcctgattggccacatgctggtgctgaaggtgctgaatttttgtctcctggtgaagcggctgttgattcctatccaaattggttgaaattccacatcggtattaaccgatacgaattgtattctagacataatcctgctattgaagctttgttgcatgatttgtctagtcagagaattacttctgttgctatgaagtctggtggtactcagttgaagttgataatgacttttcaaaactatgggcaagctttgtttaaaccaatgaaacaaacgagagaacaagaaactccacctgattttttttatttttcggattatgaaagacataatgctgaaattgctgctttccacttggacagaatattggattttcgcagagttccacctgttgctggtcggatggttaacatgactaaggaaattagagatgttactagagataaaaaattgtggagaacttttttcatttctccggctaataatatttgtttttacggggaatgttcttattattgttctactgaacatgctttgtgtggtaaacctgatcaaattgaaggttctttggctgcttttttgcctgatttgtctttggctaaaagaaaaacttggagaaatccatggagaagatcttatcataaaagaaaaaaagctgaatgggaagttgatcctgattattgtgaagaagttaaacaaactccaccatatgattcgtctcatagaatattggacgtcatggatatgacgatctttgactttctgatggggaacatggacagacatcactatgaaacattcgaaaaattcggtaatgaaacttttatcatccatttggataatggtagaggttttggtaaatattctcatgatgaattgtctattttggttccattgcaacagtgttgtagaataaggaaaagcacttacttaagattacaactcttggctaaagaagaatataaattgtctttgttgatggctgaatctttgagaggtgatcaagttgctcctgttttgtatcaaccacatttggaagctttggatagaagattgagagttgttttgaaagctgttagagattgtgttgaaagaaatggtttgcattctgttgttgatgatgatttggatactgaacatagagctgcttctgctagagattataaagatgatgatgataaatga

SEQ ID NO: 374 - DNA, 1794 bp, synthetic construct
  source 1..1794: mol_type = other DNA
atgttgctaactaaacgtttctctaagttgttcaagttgacgttcattgttttgatactctgtggtttgtttgttatcactaataaatacatggatgaaaatacttctagattggaaagaagaggtgctagaccatctggtgaacctggttgttcttgtgctcaacctgctgctgaagtcgctgctcctggttgggctcaagttagaggtagacctggtgaaccacctgctgcttcgtcagctgctggtgatgctggttggccaaataaacatactttgagaattttgcaagatttttcttctgatccatcttctaatttatctagtcactctttggaaaaattgccacctgctgctgagcctgctgaaagagctttgagaggtagagaccctggtgcgctcagaccacatgatcctgctcatagaccattgttgagagaccctggtcctagaagatctgaatctccacctggtcctggtggtgatgcttctttgttggctagattgtttgaacatccattgtatagagttgctgttccaccattgactgaagaagatgttttgtttaatgttaattctgatactagattgtctccaaaagctgctgaaaatcctgattggccacatgctggtgctgaaggtgctgaatttttgtctcctggtgaagcggctgttgattcctatccaaattggttgaaattccacatcggtattaaccgatacgaattgtattctagacataatcctgctattgaagctttgttgcatgatttgtctagtcagagaattacttctgttgctatgaagtctggtggtactcagttgaagttgataatgacttttcaaaactatgggcaagctttgtttaaaccaatgaaacaaacgagagaacaagaaactccacctgattttttttatttttcggattatgaaagacataatgctgaaattgctgctttccacttggacagaatattggattttcgcagagttccacctgttgctggtcggatggttaacatgactaaggaaattagagatgttactagagataaaaaattgtggagaacttttttcatttctccggctaataatatttgtttttacggggaatgttcttattattgttctactgaacatgctttgtgtggtaaacctgatcaaattgaaggttctttggctgcttttttgcctgatttgtctttggctaaaagaaaaacttggagaaatccatggagaagatcttatcataaaagaaaaaaagctgaatgggaagttgatcctgattattgtgaagaagttaaacaaactccaccatatgattcgtctcatagaatattggacgtcatggatatgacgatctttgactttctgatggggaacatggacagacatcactatgaaacattcgaaaaattcggtaatgaaacttttatcatccatttggataatggtagaggttttggtaaatattctcatgatgaattgtctattttggttccattgcaacagtgttgtagaataaggaaaagcacttacttaagattacaactcttggctaaagaagaatataaattgtctttgttgatggctgaatctttgagaggtgatcaagttgctcctgttttgtatcaaccacatttggaagctttggatagaagattgagagttgttttgaaagctgttagagattgtgttgaaagaaatggtttgcattctgttgttgatgatgatttggatactgaacatagagctgcttctgctagagattataaagatgatgatgataaatga

SEQ ID NO: 375 - DNA, 1977 bp, synthetic construct
  source 1..1977: mol_type = other DNA
atgttgctaactaaacgtttctctaagttgttcaagttgacgttcattgttttgatactctgtggtttgtttgttatcactaataaatacatggatgaaaatacttctgtaaaggagtataaagagtacctagacagatatgtacagtcttattccaataaatattcttcttcttctgatgctgcttctgctgatgattctactcccttgcgagacaatgatgaagccggtaacgaaaaactcaaatccttttacaataatgtttttaactttttgatggttgattcaccaagattggaaagaagaggtgctagaccatctggtgaacctggttgttcttgtgctcaacctgctgctgaagtcgctgctcctggttgggctcaagttagaggtagacctggtgaaccacctgctgcttcgtcagctgctggtgatgctggttggccaaataaacatactttgagaattttgcaagatttttcttctgatccatcttctaatttatctagtcactctttggaaaaattgccacctgctgctgagcctgctgaaagagctttgagaggtagagaccctggtgcgctcagaccacatgatcctgctcatagaccattgttgagagaccctggtcctagaagatctgaatctccacctggtcctggtggtgatgcttctttgttggctagattgtttgaacatccattgtatagagttgctgttccaccattgactgaagaagatgttttgtttaatgttaattctgatactagattgtctccaaaagctgctgaaaatcctgattggccacatgctggtgctgaaggtgctgaatttttgtctcctggtgaagcggctgttgattcctatccaaattggttgaaattccacatcggtattaaccgatacgaattgtattctagacataatcctgctattgaagctttgttgcatgatttgtctagtcagagaattacttctgttgctatgaagtctggtggtactcagttgaagttgataatgacttttcaaaactatgggcaagctttgtttaaaccaatgaaacaaacgagagaacaagaaactccacctgattttttttatttttcggattatgaaagacataatgctgaaattgctgctttccacttggacagaatattggattttcgcagagttccacctgttgctggtcggatggttaacatgactaaggaaattagagatgttactagagataaaaaattgtggagaacttttttcatttctccggctaataatatttgtttttacggggaatgttcttattattgttctactgaacatgctttgtgtggtaaacctgatcaaattgaaggttctttggctgcttttttgcctgatttgtctttggctaaaagaaaaacttggagaaatccatggagaagatcttatcataaaagaaaaaaagctgaatgggaagttgatcctgattattgtgaagaagttaaacaaactccaccatatgattcgtctcatagaatattggacgtcatggatatgacgatctttgactttctgatggggaacatggacagacatcactatgaaacattcgaaaaattcggtaatgaaacttttatcatccatttggataatggtagaggttttggtaaatattctcatgatgaattgtctattttggttccattgcaacagtgttgtagaataaggaaaagcacttacttaagattacaactcttggctaaagaagaatataaattgtctttgttgatggctgaatctttgagaggtgatcaagttgctcctgttttgtatcaaccacatttggaagctttggatagaagattgagagttgttttgaaagctgttagagattgtgttgaaagaaatggtttgcattctgttgttgatgatgatttggatactgaacatagagctgcttctgctagagattataaagatgatgatgataaatga

SEQ ID NO: 376 - DNA, 2136 bp, synthetic construct
  source 1..2136: mol_type = other DNA
atgttgctaactaaacgtttctctaagttgttcaagttgacgttcattgttttgatactctgtggtttgtttgttatcactaataaatacatggatgaaaatacttctgtaaaggagtataaagagtacctagacagatatgtacagtcttattccaataaatattcttcttcttctgatgctgcttctgctgatgattctactcccttgcgagacaatgatgaagccggtaacgaaaaactcaaatccttttacaataatgtttttaactttttgatggttgattcaccaaaaggttctactgctaaacaatataatgaagcttgtttgttgaaaggtgatattggagatagacctgatcattataaggatttgtacaaattgtctgctaaagaattgtctaaatgtttggaattgtctcctgatgaagttgcttctttgactaaatctagattggaaagaagaggtgctagaccatctggtgaacctggttgttcttgtgctcaacctgctgctgaagtcgctgctcctggttgggctcaagttagaggtagacctggtgaaccacctgctgcttcgtcagctgctggtgatgctggttggccaaataaacatactttgagaattttgcaagatttttcttctgatccatcttctaatttatctagtcactctttggaaaaattgccacctgctgctgagcctgctgaaagagctttgagaggtagagaccctggtgcgctcagaccacatgatcctgctcatagaccattgttgagagaccctggtcctagaagatctgaatctccacctggtcctggtggtgatgcttctttgttggctagattgtttgaacatccattgtatagagttgctgttccaccattgactgaagaagatgttttgtttaatgttaattctgatactagattgtctccaaaagctgctgaaaatcctgattggccacatgctggtgctgaaggtgctgaatttttgtctcctggtgaagcggctgttgattcctatccaaattggttgaaattccacatcggtattaaccgatacgaattgtattctagacataatcctgctattgaagctttgttgcatgatttgtctagtcagagaattacttctgttgctatgaagtctggtggtactcagttgaagttgataatgacttttcaaaactatgggcaagctttgtttaaaccaatgaaacaaacgagagaacaagaaactccacctgattttttttatttttcggattatgaaagacataatgctgaaattgctgctttccacttggacagaatattggattttcgcagagttccacctgttgctggtcggatggttaacatgactaaggaaattagagatgttactagagataaaaaattgtggagaacttttttcatttctccggctaataatatttgtttttacggggaatgttcttattattgttctactgaacatgctttgtgtggtaaacctgatcaaattgaaggttctttggctgcttttttgcctgatttgtctttggctaaaagaaaaacttggagaaatccatggagaagatcttatcataaaagaaaaaaagctgaatgggaagttgatcctgattattgtgaagaagttaaacaaactccaccatatgattcgtctcatagaatattggacgtcatggatatgacgatctttgactttctgatggggaacatggacagacatcactatgaaacattcgaaaaattcggtaatgaaacttttatcatccatttggataatggtagaggttttggtaaatattctcatgatgaattgtctattttggttccattgcaacagtgttgtagaataaggaaaagcacttacttaagattacaactcttggctaaagaagaatataaattgtctttgttgatggctgaatctttgagaggtgatcaagttgctcctgttttgtatcaaccacatttggaagctttggatagaagattgagagttgttttgaaagctgttagagattgtgttgaaagaaatggtttgcattctgttgttgatgatgatttggatactgaacatagagctgcttctgctagagattataaagatgatgatgataaatga

SEQ ID NO: 377 - DNA, 1812 bp, synthetic construct
  source 1..1812: mol_type = other DNA
atgttggctttgagaagatttatcttgaatcagagatctttgagatcttgtactataccaatattggttggggctttgattattattttggttttgtttcaattggtcactcatagaaacgatgctagattggaaagaagaggtgctagaccatctggtgaacctggttgttcttgtgctcaacctgctgctgaagtcgctgctcctggttgggctcaagttagaggtagacctggtgaaccacctgctgcttcgtcagctgctggtgatgctggttggccaaataaacatactttgagaattttgcaagatttttcttctgatccatcttctaatttatctagtcactctttggaaaaattgccacctgctgctgagcctgctgaaagagctttgagaggtagagaccctggtgcgctcagaccacatgatcctgctcatagaccattgttgagagaccctggtcctagaagatctgaatctccacctggtcctggtggtgatgcttctttgttggctagattgtttgaacatccattgtatagagttgctgttccaccattgactgaagaagatgttttgtttaatgttaattctgatactagattgtctccaaaagctgctgaaaatcctgattggccacatgctggtgctgaaggtgctgaatttttgtctcctggtgaagcggctgttgattcctatccaaattggttgaaattccacatcggtattaaccgatacgaattgtattctagacataatcctgctattgaagctttgttgcatgatttgtctagtcagagaattacttctgttgctatgaagtctggtggtactcagttgaagttgataatgacttttcaaaactatgggcaagctttgtttaaaccaatgaaacaaacgagagaacaagaaactccacctgattttttttatttttcggattatgaaagacataatgctgaaattgctgctttccacttggacagaatattggattttcgcagagttccacctgttgctggtcggatggttaacatgactaaggaaattagagatgttactagagataaaaaattgtggagaacttttttcatttctccggctaataatatttgtttttacggggaatgttcttattattgttctactgaacatgctttgtgtggtaaacctgatcaaattgaaggttctttggctgcttttttgcctgatttgtctttggctaaaagaaaaacttggagaaatccatggagaagatcttatcataaaagaaaaaaagctgaatgggaagttgatcctgattattgtgaagaagttaaacaaactccaccatatgattcgtctcatagaatattggacgtcatggatatgacgatctttgactttctgatggggaacatggacagacatcactatgaaacattcgaaaaattcggtaatgaaacttttatcatccatttggataatggtagaggttttggtaaatattctcatgatgaattgtctattttggttccattgcaacagtgttgtagaataaggaaaagcacttacttaagattacaactcttggctaaagaagaatataaattgtctttgttgatggctgaatctttgagaggtgatcaagttgctcctgttttgtatcaaccacatttggaagctttggatagaagattgagagttgttttgaaagctgttagagattgtgttgaaagaaatggtttgcattctgttgttgatgatgatttggatactgaacatagagctgcttctgctagagattataaagatgatgatgataaatga

SEQ ID NO: 378 - DNA, 1965 bp, synthetic construct
  source 1..1965: mol_type = other DNA
atgttggctttgagaagatttatcttgaatcagagatctttgagatcttgtactataccaatattggttggggctttgattattattttggttttgtttcaattggtcactcatagaaacgatgctctcattagatcctctaatgttaattctaccaacaaaaaaacactcaaggatgctgatccaaaagttttgattgaagcttttggttctcctgaagttgatcctgttgatactattcctgtttctccacttgaattggtgccattttacgatcaaagattggaaagaagaggtgctagaccatctggtgaacctggttgttcttgtgctcaacctgctgctgaagtcgctgctcctggttgggctcaagttagaggtagacctggtgaaccacctgctgcttcgtcagctgctggtgatgctggttggccaaataaacatactttgagaattttgcaagatttttcttctgatccatcttctaatttatctagtcactctttggaaaaattgccacctgctgctgagcctgctgaaagagctttgagaggtagagaccctggtgcgctcagaccacatgatcctgctcatagaccattgttgagagaccctggtcctagaagatctgaatctccacctggtcctggtggtgatgcttctttgttggctagattgtttgaacatccattgtatagagttgctgttccaccattgactgaagaagatgttttgtttaatgttaattctgatactagattgtctccaaaagctgctgaaaatcctgattggccacatgctggtgctgaaggtgctgaatttttgtctcctggtgaagcggctgttgattcctatccaaattggttgaaattccacatcggtattaaccgatacgaattgtattctagacataatcctgctattgaagctttgttgcatgatttgtctagtcagagaattacttctgttgctatgaagtctggtggtactcagttgaagttgataatgacttttcaaaactatgggcaagctttgtttaaaccaatgaaacaaacgagagaacaagaaactccacctgattttttttatttttcggattatgaaagacataatgctgaaattgctgctttccacttggacagaatattggattttcgcagagttccacctgttgctggtcggatggttaacatgactaaggaaattagagatgttactagagataaaaaattgtggagaacttttttcatttctccggctaataatatttgtttttacggggaatgttcttattattgttctactgaacatgctttgtgtggtaaacctgatcaaattgaaggttctttggctgcttttttgcctgatttgtctttggctaaaagaaaaacttggagaaatccatggagaagatcttatcataaaagaaaaaaagctgaatgggaagttgatcctgattattgtgaagaagttaaacaaactccaccatatgattcgtctcatagaatattggacgtcatggatatgacgatctttgactttctgatggggaacatggacagacatcactatgaaacattcgaaaaattcggtaatgaaacttttatcatccatttggataatggtagaggttttggtaaatattctcatgatgaattgtctattttggttccattgcaacagtgttgtagaataaggaaaagcacttacttaagattacaactcttggctaaagaagaatataaattgtctttgttgatggctgaatctttgagaggtgatcaagttgctcctgttttgtatcaaccacatttggaagctttggatagaagattgagagttgttttgaaagctgttagagattgtgttgaaagaaatggtttgcattctgttgttgatgatgatttggatactgaacatagagctgcttctgctagagattataaagatgatgatgataaatga

SEQ ID NO: 379 - DNA, 2145 bp, synthetic construct
  source 1..2145: mol_type = other DNA
atgttggctttgagaagatttatcttgaatcagagatctttgagatcttgtactataccaatattggttggggctttgattattattttggttttgtttcaattggtcactcatagaaacgatgctctcattagatcctctaatgttaattctaccaacaaaaaaacactcaaggatgctgatccaaaagttttgattgaagcttttggttctcctgaagttgatcctgttgatactattcctgtttctccacttgaattggtgccattttacgatcaatctattgatactaagaggtcctcttcatggttgataaacaaaaagggttattataaacacttcaacgaactgtctttgacggacagatgcaagttctattttagaacattgtatactctagacgatgagtggactaactctgttaaaaaattggaatattcaattaatgataatgaaggtagattggaaagaagaggtgctagaccatctggtgaacctggttgttcttgtgctcaacctgctgctgaagtcgctgctcctggttgggctcaagttagaggtagacctggtgaaccacctgctgcttcgtcagctgctggtgatgctggttggccaaataaacatactttgagaattttgcaagatttttcttctgatccatcttctaatttatctagtcactctttggaaaaattgccacctgctgctgagcctgctgaaagagctttgagaggtagagaccctggtgcgctcagaccacatgatcctgctcatagaccattgttgagagaccctggtcctagaagatctgaatctccacctggtcctggtggtgatgcttctttgttggctagattgtttgaacatccattgtatagagttgctgttccaccattgactgaagaagatgttttgtttaatgttaattctgatactagattgtctccaaaagctgctgaaaatcctgattggccacatgctggtgctgaaggtgctgaatttttgtctcctggtgaagcggctgttgattcctatccaaattggttgaaattccacatcggtattaaccgatacgaattgtattctagacataatcctgctattgaagctttgttgcatgatttgtctagtcagagaattacttctgttgctatgaagtctggtggtactcagttgaagttgataatgacttttcaaaactatgggcaagctttgtttaaaccaatgaaacaaacgagagaacaagaaactccacctgattttttttatttttcggattatgaaagacataatgctgaaattgctgctttccacttggacagaatattggattttcgcagagttccacctgttgctggtcggatggttaacatgactaaggaaattagagatgttactagagataaaaaattgtggagaacttttttcatttctccggctaataatatttgtttttacggggaatgttcttattattgttctactgaacatgctttgtgtggtaaacctgatcaaattgaaggttctttggctgcttttttgcctgatttgtctttggctaaaagaaaaacttggagaaatccatggagaagatcttatcataaaagaaaaaaagctgaatgggaagttgatcctgattattgtgaagaagttaaacaaactccaccatatgattcgtctcatagaatattggacgtcatggatatgacgatctttgactttctgatggggaacatggacagacatcactatgaaacattcgaaaaattcggtaatgaaacttttatcatccatttggataatggtagaggttttggtaaatattctcatgatgaattgtctattttggttccattgcaacagtgttgtagaataaggaaaagcacttacttaagattacaactcttggctaaagaagaatataaattgtctttgttgatggctgaatctttgagaggtgatcaagttgctcctgttttgtatcaaccacatttggaagctttggatagaagattgagagttgttttgaaagctgttagagattgtgttgaaagaaatggtttgcattctgttgttgatgatgatttggatactgaacatagagctgcttctgctagagattataaagatgatgatgataaatga

SEQ ID NO: 380 - DNA, 1776 bp, synthetic construct
  source 1..1776: mol_type = other DNA
atgcatgttttgttgtctaaaaagattgctagatttttgttgatatcttttgtttttgttttggctttgatggttaccattaaccatccgagattggaaagaagaggtgctagaccatctggtgaacctggttgttcttgtgctcaacctgctgctgaagtcgctgctcctggttgggctcaagttagaggtagacctggtgaaccacctgctgcttcgtcagctgctggtgatgctggttggccaaataaacatactttgagaattttgcaagatttttcttctgatccatcttctaatttatctagtcactctttggaaaaattgccacctgctgctgagcctgctgaaagagctttgagaggtagagaccctggtgcgctcagaccacatgatcctgctcatagaccattgttgagagaccctggtcctagaagatctgaatctccacctggtcctggtggtgatgcttctttgttggctagattgtttgaacatccattgtatagagttgctgttccaccattgactgaagaagatgttttgtttaatgttaattctgatactagattgtctccaaaagctgctgaaaatcctgattggccacatgctggtgctgaaggtgctgaatttttgtctcctggtgaagcggctgttgattcctatccaaattggttgaaattccacatcggtattaaccgatacgaattgtattctagacataatcctgctattgaagctttgttgcatgatttgtctagtcagagaattacttctgttgctatgaagtctggtggtactcagttgaagttgataatgacttttcaaaactatgggcaagctttgtttaaaccaatgaaacaaacgagagaacaagaaactccacctgattttttttatttttcggattatgaaagacataatgctgaaattgctgctttccacttggacagaatattggattttcgcagagttccacctgttgctggtcggatggttaacatgactaaggaaattagagatgttactagagataaaaaattgtggagaacttttttcatttctccggctaataatatttgtttttacggggaatgttcttattattgttctactgaacatgctttgtgtggtaaacctgatcaaattgaaggttctttggctgcttttttgcctgatttgtctttggctaaaagaaaaacttggagaaatccatggagaagatcttatcataaaagaaaaaaagctgaatgggaagttgatcctgattattgtgaagaagttaaacaaactccaccatatgattcgtctcatagaatattggacgtcatggatatgacgatctttgactttctgatggggaacatggacagacatcactatgaaacattcgaaaaattcggtaatgaaacttttatcatccatttggataatggtagaggttttggtaaatattctcatgatgaattgtctattttggttccattgcaacagtgttgtagaataaggaaaagcacttacttaagattacaactcttggctaaagaagaatataaattgtctttgttgatggctgaatctttgagaggtgatcaagttgctcctgttttgtatcaaccacatttggaagctttggatagaagattgagagttgttttgaaagctgttagagattgtgttgaaagaaatggtttgcattctgttgttgatgatgatttggatactgaacatagagctgcttctgctagagattataaagatgatgatgataaatga

SEQ ID NO: 381 - DNA, 1941 bp, synthetic construct
  source 1..1941: mol_type = other DNA
atgcatgttttgttgtctaaaaagattgctagatttttgttgatatcttttgtttttgttttggctttgatggttaccattaaccatccgaagactaaacaaatgtctgaacaatatgttactccatatttgccaaaatctttgcaaccaattgctaaaatttctgctgaagaacaaagaagaattcaatctgaacaagaagaagctgaattgaaacaatctttggaaggtgaagcaataagaaatgctaccgttagattggaaagaagaggtgctagaccatctggtgaacctggttgttcttgtgctcaacctgctgctgaagtcgctgctcctggttgggctcaagttagaggtagacctggtgaaccacctgctgcttcgtcagctgctggtgatgctggttggccaaataaacatactttgagaattttgcaagatttttcttctgatccatcttctaatttatctagtcactctttggaaaaattgccacctgctgctgagcctgctgaaagagctttgagaggtagagaccctggtgcgctcagaccacatgatcctgctcatagaccattgttgagagaccctggtcctagaagatctgaatctccacctggtcctggtggtgatgcttctttgttggctagattgtttgaacatccattgtatagagttgctgttccaccattgactgaagaagatgttttgtttaatgttaattctgatactagattgtctccaaaagctgctgaaaatcctgattggccacatgctggtgctgaaggtgctgaatttttgtctcctggtgaagcggctgttgattcctatccaaattggttgaaattccacatcggtattaaccgatacgaattgtattctagacataatcctgctattgaagctttgttgcatgatttgtctagtcagagaattacttctgttgctatgaagtctggtggtactcagttgaagttgataatgacttttcaaaactatgggcaagctttgtttaaaccaatgaaacaaacgagagaacaagaaactccacctgattttttttatttttcggattatgaaagacataatgctgaaattgctgctttccacttggacagaatattggattttcgcagagttccacctgttgctggtcggatggttaacatgactaaggaaattagagatgttactagagataaaaaattgtggagaacttttttcatttctccggctaataatatttgtttttacggggaatgttcttattattgttctactgaacatgctttgtgtggtaaacctgatcaaattgaaggttctttggctgcttttttgcctgatttgtctttggctaaaagaaaaacttggagaaatccatggagaagatcttatcataaaagaaaaaaagctgaatgggaagttgatcctgattattgtgaagaagttaaacaaactccaccatatgattcgtctcatagaatattggacgtcatggatatgacgatctttgactttctgatggggaacatggacagacatcactatgaaacattcgaaaaattcggtaatgaaacttttatcatccatttggataatggtagaggttttggtaaatattctcatgatgaattgtctattttggttccattgcaacagtgttgtagaataaggaaaagcacttacttaagattacaactcttggctaaagaagaatataaattgtctttgttgatggctgaatctttgagaggtgatcaagttgctcctgttttgtatcaaccacatttggaagctttggatagaagattgagagttgttttgaaagctgttagagattgtgttgaaagaaatggtttgcattctgttgttgatgatgatttggatactgaacatagagctgcttctgctagagattataaagatgatgatgataaatga

SEQ ID NO: 382 - DNA, 2166 bp, synthetic construct
  source 1..2166: mol_type = other DNA
atgcatgttttgttgtctaaaaagattgctagatttttgttgatatcttttgtttttgttttggctttgatggttaccattaaccatccgaagactaaacaaatgtctgaacaatatgttactccatatttgccaaaatctttgcaaccaattgctaaaatttctgctgaagaacaaagaagaattcaatctgaacaagaagaagctgaattgaaacaatctttggaaggtgaagcaataagaaatgctaccgttaacgccattaaagaaaaaattaaatcttatggtggtaatgaaactactttgggttttatggttccatcttatattaatcatagaggttctccacccaaagcttgcttcgtttcattgatcacagaaagggactctatgactcaaatcttgcaatctatagatgaggtccaagtcaagtttaacaaaaattttgcttatccatgggtttttatttctcaaggtgaaagattggaaagaagaggtgctagaccatctggtgaacctggttgttcttgtgctcaacctgctgctgaagtcgctgctcctggttgggctcaagttagaggtagacctggtgaaccacctgctgcttcgtcagctgctggtgatgctggttggccaaataaacatactttgagaattttgcaagatttttcttctgatccatcttctaatttatctagtcactctttggaaaaattgccacctgctgctgagcctgctgaaagagctttgagaggtagagaccctggtgcgctcagaccacatgatcctgctcatagaccattgttgagagaccctggtcctagaagatctgaatctccacctggtcctggtggtgatgcttctttgttggctagattgtttgaacatccattgtatagagttgctgttccaccattgactgaagaagatgttttgtttaatgttaattctgatactagattgtctccaaaagctgctgaaaatcctgattggccacatgctggtgctgaaggtgctgaatttttgtctcctggtgaagcggctgttgattcctatccaaattggttgaaattccacatcggtattaaccgatacgaattgtattctagacataatcctgctattgaagctttgttgcatgatttgtctagtcagagaattacttctgttgctatgaagtctggtggtactcagttgaagttgataatgacttttcaaaactatgggcaagctttgtttaaaccaatgaaacaaacgagagaacaagaaactccacctgattttttttatttttcggattatgaaagacataatgctgaaattgctgctttccacttggacagaatattggattttcgcagagttccacctgttgctggtcggatggttaacatgactaaggaaattagagatgttactagagataaaaaattgtggagaacttttttcatttctccggctaataatatttgtttttacggggaatgttcttattattgttctactgaacatgctttgtgtggtaaacctgatcaaattgaaggttctttggctgcttttttgcctgatttgtctttggctaaaagaaaaacttggagaaatccatggagaagatcttatcataaaagaaaaaaagctgaatgggaagttgatcctgattattgtgaagaagttaaacaaactccaccatatgattcgtctcatagaatattggacgtcatggatatgacgatctttgactttctgatggggaacatggacagacatcactatgaaacattcgaaaaattcggtaatgaaacttttatcatccatttggataatggtagaggttttggtaaatattctcatgatgaattgtctattttggttccattgcaacagtgttgtagaataaggaaaagcacttacttaagattacaactcttggctaaagaagaatataaattgtctttgttgatggctgaatctttgagaggtgatcaagttgctcctgttttgtatcaaccacatttggaagctttggatagaagattgagagttgttttgaaagctgttagagattgtgttgaaagaaatggtttgcattctgttgttgatgatgatttggatactgaacatagagctgcttctgctagagattataaagatgatgatgataaatga

SEQ ID NO: 383 - DNA, 1593 bp, synthetic construct
  source 1..1593: mol_type = other DNA
atgagaggtagacctggtgaaccacctgctgcttcgtcagctgctggtgatgctggttggccaaataaacatactttgagaattttgcaagatttttcttctgatccatcttctaatttatctagtcactctttggaaaaattgccacctgctgctgagcctgctgaaagagctttgagaggtagagaccctggtgcgctcagaccacatgatcctgctcatagaccattgttgagagaccctggtcctagaagatctgaatctccacctggtcctggtggtgatgcttctttgttggctagattgtttgaacatccattgtatagagttgctgttccaccattgactgaagaagatgttttgtttaatgttaattctgatactagattgtctccaaaagctgctgaaaatcctgattggccacatgctggtgctgaaggtgctgaatttttgtctcctggtgaagcggctgttgattcctatccaaattggttgaaattccacatcggtattaaccgatacgaattgtattctagacataatcctgctattgaagctttgttgcatgatttgtctagtcagagaattacttctgttgctatgaagtctggtggtactcagttgaagttgataatgacttttcaaaactatgggcaagctttgtttaaaccaatgaaacaaacgagagaacaagaaactccacctgattttttttatttttcggattatgaaagacataatgctgaaattgctgctttccacttggacagaatattggattttcgcagagttccacctgttgctggtcggatggttaacatgactaaggaaattagagatgttactagagataaaaaattgtggagaacttttttcatttctccggctaataatatttgtttttacggggaatgttcttattattgttctactgaacatgctttgtgtggtaaacctgatcaaattgaaggttctttggctgcttttttgcctgatttgtctttggctaaaagaaaaacttggagaaatccatggagaagatcttatcataaaagaaaaaaagctgaatgggaagttgatcctgattattgtgaagaagttaaacaaactccaccatatgattcgtctcatagaatattggacgtcatggatatgacgatctttgactttctgatggggaacatggacagacatcactatgaaacattcgaaaaattcggtaatgaaacttttatcatccatttggataatggtagaggttttggtaaatattctcatgatgaattgtctattttggttccattgcaacagtgttgtagaataaggaaaagcacttacttaagattacaactcttggctaaagaagaatataaattgtctttgttgatggctgaatctttgagaggtgatcaagttgctcctgttttgtatcaaccacatttggaagctttggatagaagattgagagttgttttgaaagctgttagagattgtgttgaaagaaatggtttgcattctgttgttgatgatgatttggatactgaacatagagctgcttctgctagagattataaagatgatgatgataaatga

SEQ ID NO: 384 - DNA, 1764 bp, synthetic construct
  source 1..1764: mol_type = other DNA
atggctttatttttgtctaaaagattgttgagatttactgttattgctggtgctgttattgttctattgttgacgctcaattcaaattcacgaactcaacaatatattccatcttctatttctgctgcttttgattttacttctggttctatttctcctgaacaacaagttattagaggtagacctggtgaaccacctgctgcttcgtcagctgctggtgatgctggttggccaaataaacatactttgagaattttgcaagatttttcttctgatccatcttctaatttatctagtcactctttggaaaaattgccacctgctgctgagcctgctgaaagagctttgagaggtagagaccctggtgcgctcagaccacatgatcctgctcatagaccattgttgagagaccctggtcctagaagatctgaatctccacctggtcctggtggtgatgcttctttgttggctagattgtttgaacatccattgtatagagttgctgttccaccattgactgaagaagatgttttgtttaatgttaattctgatactagattgtctccaaaagctgctgaaaatcctgattggccacatgctggtgctgaaggtgctgaatttttgtctcctggtgaagcggctgttgattcctatccaaattggttgaaattccacatcggtattaaccgatacgaattgtattctagacataatcctgctattgaagctttgttgcatgatttgtctagtcagagaattacttctgttgctatgaagtctggtggtactcagttgaagttgataatgacttttcaaaactatgggcaagctttgtttaaaccaatgaaacaaacgagagaacaagaaactccacctgattttttttatttttcggattatgaaagacataatgctgaaattgctgctttccacttggacagaatattggattttcgcagagttccacctgttgctggtcggatggttaacatgactaaggaaattagagatgttactagagataaaaaattgtggagaacttttttcatttctccggctaataatatttgtttttacggggaatgttcttattattgttctactgaacatgctttgtgtggtaaacctgatcaaattgaaggttctttggctgcttttttgcctgatttgtctttggctaaaagaaaaacttggagaaatccatggagaagatcttatcataaaagaaaaaaagctgaatgggaagttgatcctgattattgtgaagaagttaaacaaactccaccatatgattcgtctcatagaatattggacgtcatggatatgacgatctttgactttctgatggggaacatggacagacatcactatgaaacattcgaaaaattcggtaatgaaacttttatcatccatttggataatggtagaggttttggtaaatattctcatgatgaattgtctattttggttccattgcaacagtgttgtagaataaggaaaagcacttacttaagattacaactcttggctaaagaagaatataaattgtctttgttgatggctgaatctttgagaggtgatcaagttgctcctgttttgtatcaaccacatttggaagctttggatagaagattgagagttgttttgaaagctgttagagattgtgttgaaagaaatggtttgcattctgttgttgatgatgatttggatactgaacatagagctgcttctgctagagattataaagatgatgatgataaatga

SEQ ID NO: 385 - DNA, 1830 bp, synthetic construct
  source 1..1830: mol_type = other DNA
atggctttatttttgtctaaaagattgttgagatttactgttattgctggtgctgttattgttctattgttgacgctcaattcaaattcacgaactcaacaatatattccatcttctatttctgctgcttttgattttacttctggttctatttctcctgaacaacaagttatttctgaagaaaatgatgctaagaaattagaacagtccgctttgaattctgaagcttctgaagattctagaggtagacctggtgaaccacctgctgcttcgtcagctgctggtgatgctggttggccaaataaacatactttgagaattttgcaagatttttcttctgatccatcttctaatttatctagtcactctttggaaaaattgccacctgctgctgagcctgctgaaagagctttgagaggtagagaccctggtgcgctcagaccacatgatcctgctcatagaccattgttgagagaccctggtcctagaagatctgaatctccacctggtcctggtggtgatgcttctttgttggctagattgtttgaacatccattgtatagagttgctgttccaccattgactgaagaagatgttttgtttaatgttaattctgatactagattgtctccaaaagctgctgaaaatcctgattggccacatgctggtgctgaaggtgctgaatttttgtctcctggtgaagcggctgttgattcctatccaaattggttgaaattccacatcggtattaaccgatacgaattgtattctagacataatcctgctattgaagctttgttgcatgatttgtctagtcagagaattacttctgttgctatgaagtctggtggtactcagttgaagttgataatgacttttcaaaactatgggcaagctttgtttaaaccaatgaaacaaacgagagaacaagaaactccacctgattttttttatttttcggattatgaaagacataatgctgaaattgctgctttccacttggacagaatattggattttcgcagagttccacctgttgctggtcggatggttaacatgactaaggaaattagagatgttactagagataaaaaattgtggagaacttttttcatttctccggctaataatatttgtttttacggggaatgttcttattattgttctactgaacatgctttgtgtggtaaacctgatcaaattgaaggttctttggctgcttttttgcctgatttgtctttggctaaaagaaaaacttggagaaatccatggagaagatcttatcataaaagaaaaaaagctgaatgggaagttgatcctgattattgtgaagaagttaaacaaactccaccatatgattcgtctcatagaatattggacgtcatggatatgacgatctttgactttctgatggggaacatggacagacatcactatgaaacattcgaaaaattcggtaatgaaacttttatcatccatttggataatggtagaggttttggtaaatattctcatgatgaattgtctattttggttccattgcaacagtgttgtagaataaggaaaagcacttacttaagattacaactcttggctaaagaagaatataaattgtctttgttgatggctgaatctttgagaggtgatcaagttgctcctgttttgtatcaaccacatttggaagctttggatagaagattgagagttgttttgaaagctgttagagattgtgttgaaagaaatggtttgcattctgttgttgatgatgatttggatactgaacatagagctgcttctgctagagattataaagatgatgatgataaatga

SEQ ID NO: 386 - DNA, 1896 bp, synthetic construct
  source 1..1896: mol_type = other DNA
atggctttatttttgtctaaaagattgttgagatttactgttattgctggtgctgttattgttctattgttgacgctcaattcaaattcacgaactcaacaatatattccatcttctatttctgctgcttttgattttacttctggttctatttctcctgaacaacaagttatttctgaagaaaatgatgctaagaaattagaacagtccgctttgaattctgaagcttctgaagattctgaagctatggatgaagaatctaaagctttgaaagctgctgctgaaaaagctgatgctccaattgatagaggtagacctggtgaaccacctgctgcttcgtcagctgctggtgatgctggttggccaaataaacatactttgagaattttgcaagatttttcttctgatccatcttctaatttatctagtcactctttggaaaaattgccacctgctgctgagcctgctgaaagagctttgagaggtagagaccctggtgcgctcagaccacatgatcctgctcatagaccattgttgagagaccctggtcctagaagatctgaatctccacctggtcctggtggtgatgcttctttgttggctagattgtttgaacatccattgtatagagttgctgttccaccattgactgaagaagatgttttgtttaatgttaattctgatactagattgtctccaaaagctgctgaaaatcctgattggccacatgctggtgctgaaggtgctgaatttttgtctcctggtgaagcggctgttgattcctatccaaattggttgaaattccacatcggtattaaccgatacgaattgtattctagacataatcctgctattgaagctttgttgcatgatttgtctagtcagagaattacttctgttgctatgaagtctggtggtactcagttgaagttgataatgacttttcaaaactatgggcaagctttgtttaaaccaatgaaacaaacgagagaacaagaaactccacctgattttttttatttttcggattatgaaagacataatgctgaaattgctgctttccacttggacagaatattggattttcgcagagttccacctgttgctggtcggatggttaacatgactaaggaaattagagatgttactagagataaaaaattgtggagaacttttttcatttctccggctaataatatttgtttttacggggaatgttcttattattgttctactgaacatgctttgtgtggtaaacctgatcaaattgaaggttctttggctgcttttttgcctgatttgtctttggctaaaagaaaaacttggagaaatccatggagaagatcttatcataaaagaaaaaaagctgaatgggaagttgatcctgattattgtgaagaagttaaacaaactccaccatatgattcgtctcatagaatattggacgtcatggatatgacgatctttgactttctgatggggaacatggacagacatcactatgaaacattcgaaaaattcggtaatgaaacttttatcatccatttggataatggtagaggttttggtaaatattctcatgatgaattgtctattttggttccattgcaacagtgttgtagaataaggaaaagcacttacttaagattacaactcttggctaaagaagaatataaattgtctttgttgatggctgaatctttgagaggtgatcaagttgctcctgttttgtatcaaccacatttggaagctttggatagaagattgagagttgttttgaaagctgttagagattgtgttgaaagaaatggtttgcattctgttgttgatgatgatttggatactgaacatagagctgcttctgctagagattataaagatgatgatgataaatga

SEQ ID NO: 387 - DNA, 1683 bp, synthetic construct
  source 1..1683: mol_type = other DNA
atggttcatataggttttagaagcttgaaagccgttttcattttagcattgtcctcattgattttgtatggtattgttactacttttgatggtagaggtagacctggtgaaccacctgctgcttcgtcagctgctggtgatgctggttggccaaataaacatactttgagaattttgcaagatttttcttctgatccatcttctaatttatctagtcactctttggaaaaattgccacctgctgctgagcctgctgaaagagctttgagaggtagagaccctggtgcgctcagaccacatgatcctgctcatagaccattgttgagagaccctggtcctagaagatctgaatctccacctggtcctggtggtgatgcttctttgttggctagattgtttgaacatccattgtatagagttgctgttccaccattgactgaagaagatgttttgtttaatgttaattctgatactagattgtctccaaaagctgctgaaaatcctgattggccacatgctggtgctgaaggtgctgaatttttgtctcctggtgaagcggctgttgattcctatccaaattggttgaaattccacatcggtattaaccgatacgaattgtattctagacataatcctgctattgaagctttgttgcatgatttgtctagtcagagaattacttctgttgctatgaagtctggtggtactcagttgaagttgataatgacttttcaaaactatgggcaagctttgtttaaaccaatgaaacaaacgagagaacaagaaactccacctgattttttttatttttcggattatgaaagacataatgctgaaattgctgctttccacttggacagaatattggattttcgcagagttccacctgttgctggtcggatggttaacatgactaaggaaattagagatgttactagagataaaaaattgtggagaacttttttcatttctccggctaataatatttgtttttacggggaatgttcttattattgttctactgaacatgctttgtgtggtaaacctgatcaaattgaaggttctttggctgcttttttgcctgatttgtctttggctaaaagaaaaacttggagaaatccatggagaagatcttatcataaaagaaaaaaagctgaatgggaagttgatcctgattattgtgaagaagttaaacaaactccaccatatgattcgtctcatagaatattggacgtcatggatatgacgatctttgactttctgatggggaacatggacagacatcactatgaaacattcgaaaaattcggtaatgaaacttttatcatccatttggataatggtagaggttttggtaaatattctcatgatgaattgtctattttggttccattgcaacagtgttgtagaataaggaaaagcacttacttaagattacaactcttggctaaagaagaatataaattgtctttgttgatggctgaatctttgagaggtgatcaagttgctcctgttttgtatcaaccacatttggaagctttggatagaagattgagagttgttttgaaagctgttagagattgtgttgaaagaaatggtttgcattctgttgttgatgatgatttggatactgaacatagagctgcttctgctagagattataaagatgatgatgataaatga

SEQ ID NO: 388 - DNA, 1842 bp, synthetic construct
  source 1..1842: mol_type = other DNA
atggttcatataggttttagaagcttgaaagccgttttcattttagcattgtcctcattgattttgtatggtattgttactacttttgatggttctagagcttctagatatcaaccaccatatgttaatcattctcaagatccattgtaccattctggtaactcctataatagagaaaacgcgacttttgttaccttgtgtagaaatgaagatttgtattctattatccaatctatcaagaaagtcgaagacagaggtagacctggtgaaccacctgctgcttcgtcagctgctggtgatgctggttggccaaataaacatactttgagaattttgcaagatttttcttctgatccatcttctaatttatctagtcactctttggaaaaattgccacctgctgctgagcctgctgaaagagctttgagaggtagagaccctggtgcgctcagaccacatgatcctgctcatagaccattgttgagagaccctggtcctagaagatctgaatctccacctggtcctggtggtgatgcttctttgttggctagattgtttgaacatccattgtatagagttgctgttccaccattgactgaagaagatgttttgtttaatgttaattctgatactagattgtctccaaaagctgctgaaaatcctgattggccacatgctggtgctgaaggtgctgaatttttgtctcctggtgaagcggctgttgattcctatccaaattggttgaaattccacatcggtattaaccgatacgaattgtattctagacataatcctgctattgaagctttgttgcatgatttgtctagtcagagaattacttctgttgctatgaagtctggtggtactcagttgaagttgataatgacttttcaaaactatgggcaagctttgtttaaaccaatgaaacaaacgagagaacaagaaactccacctgattttttttatttttcggattatgaaagacataatgctgaaattgctgctttccacttggacagaatattggattttcgcagagttccacctgttgctggtcggatggttaacatgactaaggaaattagagatgttactagagataaaaaattgtggagaacttttttcatttctccggctaataatatttgtttttacggggaatgttcttattattgttctactgaacatgctttgtgtggtaaacctgatcaaattgaaggttctttggctgcttttttgcctgatttgtctttggctaaaagaaaaacttggagaaatccatggagaagatcttatcataaaagaaaaaaagctgaatgggaagttgatcctgattattgtgaagaagttaaacaaactccaccatatgattcgtctcatagaatattggacgtcatggatatgacgatctttgactttctgatggggaacatggacagacatcactatgaaacattcgaaaaattcggtaatgaaacttttatcatccatttggataatggtagaggttttggtaaatattctcatgatgaattgtctattttggttccattgcaacagtgttgtagaataaggaaaagcacttacttaagattacaactcttggctaaagaagaatataaattgtctttgttgatggctgaatctttgagaggtgatcaagttgctcctgttttgtatcaaccacatttggaagctttggatagaagattgagagttgttttgaaagctgttagagattgtgttgaaagaaatggtttgcattctgttgttgatgatgatttggatactgaacatagagctgcttctgctagagattataaagatgatgatgataaatga

SEQ ID NO: 389 - DNA, 2040 bp, synthetic construct
  source 1..2040: mol_type = other DNA
atggttcatataggttttagaagcttgaaagccgttttcattttagcattgtcctcattgattttgtatggtattgttactacttttgatggttctagagcttctagatatcaaccaccatatgttaatcattctcaagatccattgtaccattctggtaactcctataatagagaaaacgcgacttttgttaccttgtgtagaaatgaagatttgtattctattatccaatctatcaagaaagtcgaagaccgatttaacaacaaatttgcatacgattgggtttttctgaatgaagttccctttactgatgaatttaaagagaggacttctgttttgatttctggtcaagctaaatatggtttgattccaaaagaacattggtcttatcctgattatattgatcaagaaagagctgctgaatctagaagacaattggaagatcaacatagaggtagacctggtgaaccacctgctgcttcgtcagctgctggtgatgctggttggccaaataaacatactttgagaattttgcaagatttttcttctgatccatcttctaatttatctagtcactctttggaaaaattgccacctgctgctgagcctgctgaaagagctttgagaggtagagaccctggtgcgctcagaccacatgatcctgctcatagaccattgttgagagaccctggtcctagaagatctgaatctccacctggtcctggtggtgatgcttctttgttggctagattgtttgaacatccattgtatagagttgctgttccaccattgactgaagaagatgttttgtttaatgttaattctgatactagattgtctccaaaagctgctgaaaatcctgattggccacatgctggtgctgaaggtgctgaatttttgtctcctggtgaagcggctgttgattcctatccaaattggttgaaattccacatcggtattaaccgatacgaattgtattctagacataatcctgctattgaagctttgttgcatgatttgtctagtcagagaattacttctgttgctatgaagtctggtggtactcagttgaagttgataatgacttttcaaaactatgggcaagctttgtttaaaccaatgaaacaaacgagagaacaagaaactccacctgattttttttatttttcggattatgaaagacataatgctgaaattgctgctttccacttggacagaatattggattttcgcagagttccacctgttgctggtcggatggttaacatgactaaggaaattagagatgttactagagataaaaaattgtggagaacttttttcatttctccggctaataatatttgtttttacggggaatgttcttattattgttctactgaacatgctttgtgtggtaaacctgatcaaattgaaggttctttggctgcttttttgcctgatttgtctttggctaaaagaaaaacttggagaaatccatggagaagatcttatcataaaagaaaaaaagctgaatgggaagttgatcctgattattgtgaagaagttaaacaaactccaccatatgattcgtctcatagaatattggacgtcatggatatgacgatctttgactttctgatggggaacatggacagacatcactatgaaacattcgaaaaattcggtaatgaaacttttatcatccatttggataatggtagaggttttggtaaatattctcatgatgaattgtctattttggttccattgcaacagtgttgtagaataaggaaaagcacttacttaagattacaactcttggctaaagaagaatataaattgtctttgttgatggctgaatctttgagaggtgatcaagttgctcctgttttgtatcaaccacatttggaagctttggatagaagattgagagttgttttgaaagctgttagagattgtgttgaaagaaatggtttgcattctgttgttgatgatgatttggatactgaacatagagctgcttctgctagagattataaagatgatgatgataaatga

SEQ ID NO: 390 - DNA, 1698 bp, synthetic construct
  source 1..1698: mol_type = other DNA
atgttgctaactaaacgtttctctaagttgttcaagttgacgttcattgttttgatactctgtggtttgtttgttatcactaataaatacatggatgaaaatacttctagaggtagacctggtgaaccacctgctgcttcgtcagctgctggtgatgctggttggccaaataaacatactttgagaattttgcaagatttttcttctgatccatcttctaatttatctagtcactctttggaaaaattgccacctgctgctgagcctgctgaaagagctttgagaggtagagaccctggtgcgctcagaccacatgatcctgctcatagaccattgttgagagaccctggtcctagaagatctgaatctccacctggtcctggtggtgatgcttctttgttggctagattgtttgaacatccattgtatagagttgctgttccaccattgactgaagaagatgttttgtttaatgttaattctgatactagattgtctccaaaagctgctgaaaatcctgattggccacatgctggtgctgaaggtgctgaatttttgtctcctggtgaagcggctgttgattcctatccaaattggttgaaattccacatcggtattaaccgatacgaattgtattctagacataatcctgctattgaagctttgttgcatgatttgtctagtcagagaattacttctgttgctatgaagtctggtggtactcagttgaagttgataatgacttttcaaaactatgggcaagctttgtttaaaccaatgaaacaaacgagagaacaagaaactccacctgattttttttatttttcggattatgaaagacataatgctgaaattgctgctttccacttggacagaatattggattttcgcagagttccacctgttgctggtcggatggttaacatgactaaggaaattagagatgttactagagataaaaaattgtggagaacttttttcatttctccggctaataatatttgtttttacggggaatgttcttattattgttctactgaacatgctttgtgtggtaaacctgatcaaattgaaggttctttggctgcttttttgcctgatttgtctttggctaaaagaaaaacttggagaaatccatggagaagatcttatcataaaagaaaaaaagctgaatgggaagttgatcctgattattgtgaagaagttaaacaaactccaccatatgattcgtctcatagaatattggacgtcatggatatgacgatctttgactttctgatggggaacatggacagacatcactatgaaacattcgaaaaattcggtaatgaaacttttatcatccatttggataatggtagaggttttggtaaatattctcatgatgaattgtctattttggttccattgcaacagtgttgtagaataaggaaaagcacttacttaagattacaactcttggctaaagaagaatataaattgtctttgttgatggctgaatctttgagaggtgatcaagttgctcctgttttgtatcaaccacatttggaagctttggatagaagattgagagttgttttgaaagctgttagagattgtgttgaaagaaatggtttgcattctgttgttgatgatgatttggatactgaacatagagctgcttctgctagagattataaagatgatgatgataaatga

SEQ ID NO: 391 - DNA, 1881 bp, synthetic construct
  source 1..1881: mol_type = other DNA
atgttgctaactaaacgtttctctaagttgttcaagttgacgttcattgttttgatactctgtggtttgtttgttatcactaataaatacatggatgaaaatacttctgtaaaggagtataaagagtacctagacagatatgtacagtcttattccaataaatattcttcttcttctgatgctgcttctgctgatgattctactcccttgcgagacaatgatgaagccggtaacgaaaaactcaaatccttttacaataatgtttttaactttttgatggttgattcaccaagaggtagacctggtgaaccacctgctgcttcgtcagctgctggtgatgctggttggccaaataaacatactttgagaattttgcaagatttttcttctgatccatcttctaatttatctagtcactctttggaaaaattgccacctgctgctgagcctgctgaaagagctttgagaggtagagaccctggtgcgctcagaccacatgatcctgctcatagaccattgttgagagaccctggtcctagaagatctgaatctccacctggtcctggtggtgatgcttctttgttggctagattgtttgaacatccattgtatagagttgctgttccaccattgactgaagaagatgttttgtttaatgttaattctgatactagattgtctccaaaagctgctgaaaatcctgattggccacatgctggtgctgaaggtgctgaatttttgtctcctggtgaagcggctgttgattcctatccaaattggttgaaattccacatcggtattaaccgatacgaattgtattctagacataatcctgctattgaagctttgttgcatgatttgtctagtcagagaattacttctgttgctatgaagtctggtggtactcagttgaagttgataatgacttttcaaaactatgggcaagctttgtttaaaccaatgaaacaaacgagagaacaagaaactccacctgattttttttatttttcggattatgaaagacataatgctgaaattgctgctttccacttggacagaatattggattttcgcagagttccacctgttgctggtcggatggttaacatgactaaggaaattagagatgttactagagataaaaaattgtggagaacttttttcatttctccggctaataatatttgtttttacggggaatgttcttattattgttctactgaacatgctttgtgtggtaaacctgatcaaattgaaggttctttggctgcttttttgcctgatttgtctttggctaaaagaaaaacttggagaaatccatggagaagatcttatcataaaagaaaaaaagctgaatgggaagttgatcctgattattgtgaagaagttaaacaaactccaccatatgattcgtctcatagaatattggacgtcatggatatgacgatctttgactttctgatggggaacatggacagacatcactatgaaacattcgaaaaattcggtaatgaaacttttatcatccatttggataatggtagaggttttggtaaatattctcatgatgaattgtctattttggttccattgcaacagtgttgtagaataaggaaaagcacttacttaagattacaactcttggctaaagaagaatataaattgtctttgttgatggctgaatctttgagaggtgatcaagttgctcctgttttgtatcaaccacatttggaagctttggatagaagattgagagttgttttgaaagctgttagagattgtgttgaaagaaatggtttgcattctgttgttgatgatgatttggatactgaacatagagctgcttctgctagagattataaagatgatgatgataaatga

SEQ ID NO: 392 - DNA, 2040 bp, synthetic construct
  source 1..2040: mol_type = other DNA
atgttgctaactaaacgtttctctaagttgttcaagttgacgttcattgttttgatactctgtggtttgtttgttatcactaataaatacatggatgaaaatacttctgtaaaggagtataaagagtacctagacagatatgtacagtcttattccaataaatattcttcttcttctgatgctgcttctgctgatgattctactcccttgcgagacaatgatgaagccggtaacgaaaaactcaaatccttttacaataatgtttttaactttttgatggttgattcaccaaaaggttctactgctaaacaatataatgaagcttgtttgttgaaaggtgatattggagatagacctgatcattataaggatttgtacaaattgtctgctaaagaattgtctaaatgtttggaattgtctcctgatgaagttgcttctttgactaaatctagaggtagacctggtgaaccacctgctgcttcgtcagctgctggtgatgctggttggccaaataaacatactttgagaattttgcaagatttttcttctgatccatcttctaatttatctagtcactctttggaaaaattgccacctgctgctgagcctgctgaaagagctttgagaggtagagaccctggtgcgctcagaccacatgatcctgctcatagaccattgttgagagaccctggtcctagaagatctgaatctccacctggtcctggtggtgatgcttctttgttggctagattgtttgaacatccattgtatagagttgctgttccaccattgactgaagaagatgttttgtttaatgttaattctgatactagattgtctccaaaagctgctgaaaatcctgattggccacatgctggtgctgaaggtgctgaatttttgtctcctggtgaagcggctgttgattcctatccaaattggttgaaattccacatcggtattaaccgatacgaattgtattctagacataatcctgctattgaagctttgttgcatgatttgtctagtcagagaattacttctgttgctatgaagtctggtggtactcagttgaagttgataatgacttttcaaaactatgggcaagctttgtttaaaccaatgaaacaaacgagagaacaagaaactccacctgattttttttatttttcggattatgaaagacataatgctgaaattgctgctttccacttggacagaatattggattttcgcagagttccacctgttgctggtcggatggttaacatgactaaggaaattagagatgttactagagataaaaaattgtggagaacttttttcatttctccggctaataatatttgtttttacggggaatgttcttattattgttctactgaacatgctttgtgtggtaaacctgatcaaattgaaggttctttggctgcttttttgcctgatttgtctttggctaaaagaaaaacttggagaaatccatggagaagatcttatcataaaagaaaaaaagctgaatgggaagttgatcctgattattgtgaagaagttaaacaaactccaccatatgattcgtctcatagaatattggacgtcatggatatgacgatctttgactttctgatggggaacatggacagacatcactatgaaacattcgaaaaattcggtaatgaaacttttatcatccatttggataatggtagaggttttggtaaatattctcatgatgaattgtctattttggttccattgcaacagtgttgtagaataaggaaaagcacttacttaagattacaactcttggctaaagaagaatataaattgtctttgttgatggctgaatctttgagaggtgatcaagttgctcctgttttgtatcaaccacatttggaagctttggatagaagattgagagttgttttgaaagctgttagagattgtgttgaaagaaatggtttgcattctgttgttgatgatgatttggatactgaacatagagctgcttctgctagagattataaagatgatgatgataaatga

SEQ ID NO: 393 - DNA, 1716 bp, synthetic construct
  source 1..1716: mol_type = other DNA
atgttggctttgagaagatttatcttgaatcagagatctttgagatcttgtactataccaatattggttggggctttgattattattttggttttgtttcaattggtcactcatagaaacgatgctagaggtagacctggtgaaccacctgctgcttcgtcagctgctggtgatgctggttggccaaataaacatactttgagaattttgcaagatttttcttctgatccatcttctaatttatctagtcactctttggaaaaattgccacctgctgctgagcctgctgaaagagctttgagaggtagagaccctggtgcgctcagaccacatgatcctgctcatagaccattgttgagagaccctggtcctagaagatctgaatctccacctggtcctggtggtgatgcttctttgttggctagattgtttgaacatccattgtatagagttgctgttccaccattgactgaagaagatgttttgtttaatgttaattctgatactagattgtctccaaaagctgctgaaaatcctgattggccacatgctggtgctgaaggtgctgaatttttgtctcctggtgaagcggctgttgattcctatccaaattggttgaaattccacatcggtattaaccgatacgaattgtattctagacataatcctgctattgaagctttgttgcatgatttgtctagtcagagaattacttctgttgctatgaagtctggtggtactcagttgaagttgataatgacttttcaaaactatgggcaagctttgtttaaaccaatgaaacaaacgagagaacaagaaactccacctgattttttttatttttcggattatgaaagacataatgctgaaattgctgctttccacttggacagaatattggattttcgcagagttccacctgttgctggtcggatggttaacatgactaaggaaattagagatgttactagagataaaaaattgtggagaacttttttcatttctccggctaataatatttgtttttacggggaatgttcttattattgttctactgaacatgctttgtgtggtaaacctgatcaaattgaaggttctttggctgcttttttgcctgatttgtctttggctaaaagaaaaacttggagaaatccatggagaagatcttatcataaaagaaaaaaagctgaatgggaagttgatcctgattattgtgaagaagttaaacaaactccaccatatgattcgtctcatagaatattggacgtcatggatatgacgatctttgactttctgatggggaacatggacagacatcactatgaaacattcgaaaaattcggtaatgaaacttttatcatccatttggataatggtagaggttttggtaaatattctcatgatgaattgtctattttggttccattgcaacagtgttgtagaataaggaaaagcacttacttaagattacaactcttggctaaagaagaatataaattgtctttgttgatggctgaatctttgagaggtgatcaagttgctcctgttttgtatcaaccacatttggaagctttggatagaagattgagagttgttttgaaagctgttagagattgtgttgaaagaaatggtttgcattctgttgttgatgatgatttggatactgaacatagagctgcttctgctagagattataaagatgatgatgataaatga

SEQ ID NO: 394 - DNA, 1869 bp, synthetic construct
  source 1..1869: mol_type = other DNA
atgttggctttgagaagatttatcttgaatcagagatctttgagatcttgtactataccaatattggttggggctttgattattattttggttttgtttcaattggtcactcatagaaacgatgctctcattagatcctctaatgttaattctaccaacaaaaaaacactcaaggatgctgatccaaaagttttgattgaagcttttggttctcctgaagttgatcctgttgatactattcctgtttctccacttgaattggtgccattttacgatcaaagaggtagacctggtgaaccacctgctgcttcgtcagctgctggtgatgctggttggccaaataaacatactttgagaattttgcaagatttttcttctgatccatcttctaatttatctagtcactctttggaaaaattgccacctgctgctgagcctgctgaaagagctttgagaggtagagaccctggtgcgctcagaccacatgatcctgctcatagaccattgttgagagaccctggtcctagaagatctgaatctccacctggtcctggtggtgatgcttctttgttggctagattgtttgaacatccattgtatagagttgctgttccaccattgactgaagaagatgttttgtttaatgttaattctgatactagattgtctccaaaagctgctgaaaatcctgattggccacatgctggtgctgaaggtgctgaatttttgtctcctggtgaagcggctgttgattcctatccaaattggttgaaattccacatcggtattaaccgatacgaattgtattctagacataatcctgctattgaagctttgttgcatgatttgtctagtcagagaattacttctgttgctatgaagtctggtggtactcagttgaagttgataatgacttttcaaaactatgggcaagctttgtttaaaccaatgaaacaaacgagagaacaagaaactccacctgattttttttatttttcggattatgaaagacataatgctgaaattgctgctttccacttggacagaatattggattttcgcagagttccacctgttgctggtcggatggttaacatgactaaggaaattagagatgttactagagataaaaaattgtggagaacttttttcatttctccggctaataatatttgtttttacggggaatgttcttattattgttctactgaacatgctttgtgtggtaaacctgatcaaattgaaggttctttggctgcttttttgcctgatttgtctttggctaaaagaaaaacttggagaaatccatggagaagatcttatcataaaagaaaaaaagctgaatgggaagttgatcctgattattgtgaagaagttaaacaaactccaccatatgattcgtctcatagaatattggacgtcatggatatgacgatctttgactttctgatggggaacatggacagacatcactatgaaacattcgaaaaattcggtaatgaaacttttatcatccatttggataatggtagaggttttggtaaatattctcatgatgaattgtctattttggttccattgcaacagtgttgtagaataaggaaaagcacttacttaagattacaactcttggctaaagaagaatataaattgtctttgttgatggctgaatctttgagaggtgatcaagttgctcctgttttgtatcaaccacatttggaagctttggatagaagattgagagttgttttgaaagctgttagagattgtgttgaaagaaatggtttgcattctgttgttgatgatgatttggatactgaacatagagctgcttctgctagagattataaagatgatgatgataaatga

SEQ ID NO: 395 - DNA, 2049 bp, synthetic construct
  source 1..2049: mol_type = other DNA
atgttggctttgagaagatttatcttgaatcagagatctttgagatcttgtactataccaatattggttggggctttgattattattttggttttgtttcaattggtcactcatagaaacgatgctctcattagatcctctaatgttaattctaccaacaaaaaaacactcaaggatgctgatccaaaagttttgattgaagcttttggttctcctgaagttgatcctgttgatactattcctgtttctccacttgaattggtgccattttacgatcaatctattgatactaagaggtcctcttcatggttgataaacaaaaagggttattataaacacttcaacgaactgtctttgacggacagatgcaagttctattttagaacattgtatactctagacgatgagtggactaactctgttaaaaaattggaatattcaattaatgataatgaaggtagaggtagacctggtgaaccacctgctgcttcgtcagctgctggtgatgctggttggccaaataaacatactttgagaattttgcaagatttttcttctgatccatcttctaatttatctagtcactctttggaaaaattgccacctgctgctgagcctgctgaaagagctttgagaggtagagaccctggtgcgctcagaccacatgatcctgctcatagaccattgttgagagaccctggtcctagaagatctgaatctccacctggtcctggtggtgatgcttctttgttggctagattgtttgaacatccattgtatagagttgctgttccaccattgactgaagaagatgttttgtttaatgttaattctgatactagattgtctccaaaagctgctgaaaatcctgattggccacatgctggtgctgaaggtgctgaatttttgtctcctggtgaagcggctgttgattcctatccaaattggttgaaattccacatcggtattaaccgatacgaattgtattctagacataatcctgctattgaagctttgttgcatgatttgtctagtcagagaattacttctgttgctatgaagtctggtggtactcagttgaagttgataatgacttttcaaaactatgggcaagctttgtttaaaccaatgaaacaaacgagagaacaagaaactccacctgattttttttatttttcggattatgaaagacataatgctgaaattgctgctttccacttggacagaatattggattttcgcagagttccacctgttgctggtcggatggttaacatgactaaggaaattagagatgttactagagataaaaaattgtggagaacttttttcatttctccggctaataatatttgtttttacggggaatgttcttattattgttctactgaacatgctttgtgtggtaaacctgatcaaattgaaggttctttggctgcttttttgcctgatttgtctttggctaaaagaaaaacttggagaaatccatggagaagatcttatcataaaagaaaaaaagctgaatgggaagttgatcctgattattgtgaagaagttaaacaaactccaccatatgattcgtctcatagaatattggacgtcatggatatgacgatctttgactttctgatggggaacatggacagacatcactatgaaacattcgaaaaattcggtaatgaaacttttatcatccatttggataatggtagaggttttggtaaatattctcatgatgaattgtctattttggttccattgcaacagtgttgtagaataaggaaaagcacttacttaagattacaactcttggctaaagaagaatataaattgtctttgttgatggctgaatctttgagaggtgatcaagttgctcctgttttgtatcaaccacatttggaagctttggatagaagattgagagttgttttgaaagctgttagagattgtgttgaaagaaatggtttgcattctgttgttgatgatgatttggatactgaacatagagctgcttctgctagagattataaagatgatgatgataaatga

SEQ ID NO: 396 - DNA, 1680 bp, synthetic construct
  source 1..1680: mol_type = other DNA
atgcatgttttgttgtctaaaaagattgctagatttttgttgatatcttttgtttttgttttggctttgatggttaccattaaccatccgagaggtagacctggtgaaccacctgctgcttcgtcagctgctggtgatgctggttggccaaataaacatactttgagaattttgcaagatttttcttctgatccatcttctaatttatctagtcactctttggaaaaattgccacctgctgctgagcctgctgaaagagctttgagaggtagagaccctggtgcgctcagaccacatgatcctgctcatagaccattgttgagagaccctggtcctagaagatctgaatctccacctggtcctggtggtgatgcttctttgttggctagattgtttgaacatccattgtatagagttgctgttccaccattgactgaagaagatgttttgtttaatgttaattctgatactagattgtctccaaaagctgctgaaaatcctgattggccacatgctggtgctgaaggtgctgaatttttgtctcctggtgaagcggctgttgattcctatccaaattggttgaaattccacatcggtattaaccgatacgaattgtattctagacataatcctgctattgaagctttgttgcatgatttgtctagtcagagaattacttctgttgctatgaagtctggtggtactcagttgaagttgataatgacttttcaaaactatgggcaagctttgtttaaaccaatgaaacaaacgagagaacaagaaactccacctgattttttttatttttcggattatgaaagacataatgctgaaattgctgctttccacttggacagaatattggattttcgcagagttccacctgttgctggtcggatggttaacatgactaaggaaattagagatgttactagagataaaaaattgtggagaacttttttcatttctccggctaataatatttgtttttacggggaatgttcttattattgttctactgaacatgctttgtgtggtaaacctgatcaaattgaaggttctttggctgcttttttgcctgatttgtctttggctaaaagaaaaacttggagaaatccatggagaagatcttatcataaaagaaaaaaagctgaatgggaagttgatcctgattattgtgaagaagttaaacaaactccaccatatgattcgtctcatagaatattggacgtcatggatatgacgatctttgactttctgatggggaacatggacagacatcactatgaaacattcgaaaaattcggtaatgaaacttttatcatccatttggataatggtagaggttttggtaaatattctcatgatgaattgtctattttggttccattgcaacagtgttgtagaataaggaaaagcacttacttaagattacaactcttggctaaagaagaatataaattgtctttgttgatggctgaatctttgagaggtgatcaagttgctcctgttttgtatcaaccacatttggaagctttggatagaagattgagagttgttttgaaagctgttagagattgtgttgaaagaaatggtttgcattctgttgttgatgatgatttggatactgaacatagagctgcttctgctagagattataaagatgatgatgataaatga

SEQ ID NO: 397 - DNA, 1845 bp, synthetic construct
  source 1..1845: mol_type = other DNA
atgcatgttttgttgtctaaaaagattgctagatttttgttgatatcttttgtttttgttttggctttgatggttaccattaaccatccgaagactaaacaaatgtctgaacaatatgttactccatatttgccaaaatctttgcaaccaattgctaaaatttctgctgaagaacaaagaagaattcaatctgaacaagaagaagctgaattgaaacaatctttggaaggtgaagcaataagaaatgctaccgttagaggtagacctggtgaaccacctgctgcttcgtcagctgctggtgatgctggttggccaaataaacatactttgagaattttgcaagatttttcttctgatccatcttctaatttatctagtcactctttggaaaaattgccacctgctgctgagcctgctgaaagagctttgagaggtagagaccctggtgcgctcagaccacatgatcctgctcatagaccattgttgagagaccctggtcctagaagatctgaatctccacctggtcctggtggtgatgcttctttgttggctagattgtttgaacatccattgtatagagttgctgttccaccattgactgaagaagatgttttgtttaatgttaattctgatactagattgtctccaaaagctgctgaaaatcctgattggccacatgctggtgctgaaggtgctgaatttttgtctcctggtgaagcggctgttgattcctatccaaattggttgaaattccacatcggtattaaccgatacgaattgtattctagacataatcctgctattgaagctttgttgcatgatttgtctagtcagagaattacttctgttgctatgaagtctggtggtactcagttgaagttgataatgacttttcaaaactatgggcaagctttgtttaaaccaatgaaacaaacgagagaacaagaaactccacctgattttttttatttttcggattatgaaagacataatgctgaaattgctgctttccacttggacagaatattggattttcgcagagttccacctgttgctggtcggatggttaacatgactaaggaaattagagatgttactagagataaaaaattgtggagaacttttttcatttctccggctaataatatttgtttttacggggaatgttcttattattgttctactgaacatgctttgtgtggtaaacctgatcaaattgaaggttctttggctgcttttttgcctgatttgtctttggctaaaagaaaaacttggagaaatccatggagaagatcttatcataaaagaaaaaaagctgaatgggaagttgatcctgattattgtgaagaagttaaacaaactccaccatatgattcgtctcatagaatattggacgtcatggatatgacgatctttgactttctgatggggaacatggacagacatcactatgaaacattcgaaaaattcggtaatgaaacttttatcatccatttggataatggtagaggttttggtaaatattctcatgatgaattgtctattttggttccattgcaacagtgttgtagaataaggaaaagcacttacttaagattacaactcttggctaaagaagaatataaattgtctttgttgatggctgaatctttgagaggtgatcaagttgctcctgttttgtatcaaccacatttggaagctttggatagaagattgagagttgttttgaaagctgttagagattgtgttgaaagaaatggtttgcattctgttgttgatgatgatttggatactgaacatagagctgcttctgctagagattataaagatgatgatgataaatga

SEQ ID NO: 398 - DNA, 2070 bp, synthetic construct
  source 1..2070: mol_type = other DNA
atgcatgttttgttgtctaaaaagattgctagatttttgttgatatcttttgtttttgttttggctttgatggttaccattaaccatccgaagactaaacaaatgtctgaacaatatgttactccatatttgccaaaatctttgcaaccaattgctaaaatttctgctgaagaacaaagaagaattcaatctgaacaagaagaagctgaattgaaacaatctttggaaggtgaagcaataagaaatgctaccgttaacgccattaaagaaaaaattaaatcttatggtggtaatgaaactactttgggttttatggttccatcttatattaatcatagaggttctccacccaaagcttgcttcgtttcattgatcacagaaagggactctatgactcaaatcttgcaatctatagatgaggtccaagtcaagtttaacaaaaattttgcttatccatgggtttttatttctcaaggtgaaagaggtagacctggtgaaccacctgctgcttcgtcagctgctggtgatgctggttggccaaataaacatactttgagaattttgcaagatttttcttctgatccatcttctaatttatctagtcactctttggaaaaattgccacctgctgctgagcctgctgaaagagctttgagaggtagagaccctggtgcgctcagaccacatgatcctgctcatagaccattgttgagagaccctggtcctagaagatctgaatctccacctggtcctggtggtgatgcttctttgttggctagattgtttgaacatccattgtatagagttgctgttccaccattgactgaagaagatgttttgtttaatgttaattctgatactagattgtctccaaaagctgctgaaaatcctgattggccacatgctggtgctgaaggtgctgaatttttgtctcctggtgaagcggctgttgattcctatccaaattggttgaaattccacatcggtattaaccgatacgaattgtattctagacataatcctgctattgaagctttgttgcatgatttgtctagtcagagaattacttctgttgctatgaagtctggtggtactcagttgaagttgataatgacttttcaaaactatgggcaagctttgtttaaaccaatgaaacaaacgagagaacaagaaactccacctgattttttttatttttcggattatgaaagacataatgctgaaattgctgctttccacttggacagaatattggattttcgcagagttccacctgttgctggtcggatggttaacatgactaaggaaattagagatgttactagagataaaaaattgtggagaacttttttcatttctccggctaataatatttgtttttacggggaatgttcttattattgttctactgaacatgctttgtgtggtaaacctgatcaaattgaaggttctttggctgcttttttgcctgatttgtctttggctaaaagaaaaacttggagaaatccatggagaagatcttatcataaaagaaaaaaagctgaatgggaagttgatcctgattattgtgaagaagttaaacaaactccaccatatgattcgtctcatagaatattggacgtcatggatatgacgatctttgactttctgatggggaacatggacagacatcactatgaaacattcgaaaaattcggtaatgaaacttttatcatccatttggataatggtagaggttttggtaaatattctcatgatgaattgtctattttggttccattgcaacagtgttgtagaataaggaaaagcacttacttaagattacaactcttggctaaagaagaatataaattgtctttgttgatggctgaatctttgagaggtgatcaagttgctcctgttttgtatcaaccacatttggaagctttggatagaagattgagagttgttttgaaagctgttagagattgtgttgaaagaaatggtttgcattctgttgttgatgatgatttggatactgaacatagagctgcttctgctagagattataaagatgatgatgataaatga

SEQ ID NO: 399 - DNA, 1506 bp, synthetic construct
  source 1..1506: mol_type = other DNA
atggatttttcttctgatccatcttctaatttatctagtcactctttggaaaaattgccacctgctgctgagcctgctgaaagagctttgagaggtagagaccctggtgcgctcagaccacatgatcctgctcatagaccattgttgagagaccctggtcctagaagatctgaatctccacctggtcctggtggtgatgcttctttgttggctagattgtttgaacatccattgtatagagttgctgttccaccattgactgaagaagatgttttgtttaatgttaattctgatactagattgtctccaaaagctgctgaaaatcctgattggccacatgctggtgctgaaggtgctgaatttttgtctcctggtgaagcggctgttgattcctatccaaattggttgaaattccacatcggtattaaccgatacgaattgtattctagacataatcctgctattgaagctttgttgcatgatttgtctagtcagagaattacttctgttgctatgaagtctggtggtactcagttgaagttgataatgacttttcaaaactatgggcaagctttgtttaaaccaatgaaacaaacgagagaacaagaaactccacctgattttttttatttttcggattatgaaagacataatgctgaaattgctgctttccacttggacagaatattggattttcgcagagttccacctgttgctggtcggatggttaacatgactaaggaaattagagatgttactagagataaaaaattgtggagaacttttttcatttctccggctaataatatttgtttttacggggaatgttcttattattgttctactgaacatgctttgtgtggtaaacctgatcaaattgaaggttctttggctgcttttttgcctgatttgtctttggctaaaagaaaaacttggagaaatccatggagaagatcttatcataaaagaaaaaaagctgaatgggaagttgatcctgattattgtgaagaagttaaacaaactccaccatatgattcgtctcatagaatattggacgtcatggatatgacgatctttgactttctgatggggaacatggacagacatcactatgaaacattcgaaaaattcggtaatgaaacttttatcatccatttggataatggtagaggttttggtaaatattctcatgatgaattgtctattttggttccattgcaacagtgttgtagaataaggaaaagcacttacttaagattacaactcttggctaaagaagaatataaattgtctttgttgatggctgaatctttgagaggtgatcaagttgctcctgttttgtatcaaccacatttggaagctttggatagaagattgagagttgttttgaaagctgttagagattgtgttgaaagaaatggtttgcattctgttgttgatgatgatttggatactgaacatagagctgcttctgctagagattataaagatgatgatgataaataa

SEQ ID NO: 400 - DNA, 1677 bp, synthetic construct
  source 1..1677: mol_type = other DNA
atggctttatttttgtctaaaagattgttgagatttactgttattgctggtgctgttattgttctattgttgacgctcaattcaaattcacgaactcaacaatatattccatcttctatttctgctgcttttgattttacttctggttctatttctcctgaacaacaagttattgatttttcttctgatccatcttctaatttatctagtcactctttggaaaaattgccacctgctgctgagcctgctgaaagagctttgagaggtagagaccctggtgcgctcagaccacatgatcctgctcatagaccattgttgagagaccctggtcctagaagatctgaatctccacctggtcctggtggtgatgcttctttgttggctagattgtttgaacatccattgtatagagttgctgttccaccattgactgaagaagatgttttgtttaatgttaattctgatactagattgtctccaaaagctgctgaaaatcctgattggccacatgctggtgctgaaggtgctgaatttttgtctcctggtgaagcggctgttgattcctatccaaattggttgaaattccacatcggtattaaccgatacgaattgtattctagacataatcctgctattgaagctttgttgcatgatttgtctagtcagagaattacttctgttgctatgaagtctggtggtactcagttgaagttgataatgacttttcaaaactatgggcaagctttgtttaaaccaatgaaacaaacgagagaacaagaaactccacctgattttttttatttttcggattatgaaagacataatgctgaaattgctgctttccacttggacagaatattggattttcgcagagttccacctgttgctggtcggatggttaacatgactaaggaaattagagatgttactagagataaaaaattgtggagaacttttttcatttctccggctaataatatttgtttttacggggaatgttcttattattgttctactgaacatgctttgtgtggtaaacctgatcaaattgaaggttctttggctgcttttttgcctgatttgtctttggctaaaagaaaaacttggagaaatccatggagaagatcttatcataaaagaaaaaaagctgaatgggaagttgatcctgattattgtgaagaagttaaacaaactccaccatatgattcgtctcatagaatattggacgtcatggatatgacgatctttgactttctgatggggaacatggacagacatcactatgaaacattcgaaaaattcggtaatgaaacttttatcatccatttggataatggtagaggttttggtaaatattctcatgatgaattgtctattttggttccattgcaacagtgttgtagaataaggaaaagcacttacttaagattacaactcttggctaaagaagaatataaattgtctttgttgatggctgaatctttgagaggtgatcaagttgctcctgttttgtatcaaccacatttggaagctttggatagaagattgagagttgttttgaaagctgttagagattgtgttgaaagaaatggtttgcattctgttgttgatgatgatttggatactgaacatagagctgcttctgctagagattataaagatgatgatgataaataa

SEQ ID NO: 401 - DNA, 1743 bp, synthetic construct
  source 1..1743: mol_type = other DNA
atggctttatttttgtctaaaagattgttgagatttactgttattgctggtgctgttattgttctattgttgacgctcaattcaaattcacgaactcaacaatatattccatcttctatttctgctgcttttgattttacttctggttctatttctcctgaacaacaagttatttctgaagaaaatgatgctaagaaattagaacagtccgctttgaattctgaagcttctgaagattctgatttttcttctgatccatcttctaatttatctagtcactctttggaaaaattgccacctgctgctgagcctgctgaaagagctttgagaggtagagaccctggtgcgctcagaccacatgatcctgctcatagaccattgttgagagaccctggtcctagaagatctgaatctccacctggtcctggtggtgatgcttctttgttggctagattgtttgaacatccattgtatagagttgctgttccaccattgactgaagaagatgttttgtttaatgttaattctgatactagattgtctccaaaagctgctgaaaatcctgattggccacatgctggtgctgaaggtgctgaatttttgtctcctggtgaagcggctgttgattcctatccaaattggttgaaattccacatcggtattaaccgatacgaattgtattctagacataatcctgctattgaagctttgttgcatgatttgtctagtcagagaattacttctgttgctatgaagtctggtggtactcagttgaagttgataatgacttttcaaaactatgggcaagctttgtttaaaccaatgaaacaaacgagagaacaagaaactccacctgattttttttatttttcggattatgaaagacataatgctgaaattgctgctttccacttggacagaatattggattttcgcagagttccacctgttgctggtcggatggttaacatgactaaggaaattagagatgttactagagataaaaaattgtggagaacttttttcatttctccggctaataatatttgtttttacggggaatgttcttattattgttctactgaacatgctttgtgtggtaaacctgatcaaattgaaggttctttggctgcttttttgcctgatttgtctttggctaaaagaaaaacttggagaaatccatggagaagatcttatcataaaagaaaaaaagctgaatgggaagttgatcctgattattgtgaagaagttaaacaaactccaccatatgattcgtctcatagaatattggacgtcatggatatgacgatctttgactttctgatggggaacatggacagacatcactatgaaacattcgaaaaattcggtaatgaaacttttatcatccatttggataatggtagaggttttggtaaatattctcatgatgaattgtctattttggttccattgcaacagtgttgtagaataaggaaaagcacttacttaagattacaactcttggctaaagaagaatataaattgtctttgttgatggctgaatctttgagaggtgatcaagttgctcctgttttgtatcaaccacatttggaagctttggatagaagattgagagttgttttgaaagctgttagagattgtgttgaaagaaatggtttgcattctgttgttgatgatgatttggatactgaacatagagctgcttctgctagagattataaagatgatgatgataaataa

SEQ ID NO: 402 - DNA, 1809 bp, synthetic construct
  source 1..1809: mol_type = other DNA
atggctttatttttgtctaaaagattgttgagatttactgttattgctggtgctgttattgttctattgttgacgctcaattcaaattcacgaactcaacaatatattccatcttctatttctgctgcttttgattttacttctggttctatttctcctgaacaacaagttatttctgaagaaaatgatgctaagaaattagaacagtccgctttgaattctgaagcttctgaagattctgaagctatggatgaagaatctaaagctttgaaagctgctgctgaaaaagctgatgctccaattgatgatttttcttctgatccatcttctaatttatctagtcactctttggaaaaattgccacctgctgctgagcctgctgaaagagctttgagaggtagagaccctggtgcgctcagaccacatgatcctgctcatagaccattgttgagagaccctggtcctagaagatctgaatctccacctggtcctggtggtgatgcttctttgttggctagattgtttgaacatccattgtatagagttgctgttccaccattgactgaagaagatgttttgtttaatgttaattctgatactagattgtctccaaaagctgctgaaaatcctgattggccacatgctggtgctgaaggtgctgaatttttgtctcctggtgaagcggctgttgattcctatccaaattggttgaaattccacatcggtattaaccgatacgaattgtattctagacataatcctgctattgaagctttgttgcatgatttgtctagtcagagaattacttctgttgctatgaagtctggtggtactcagttgaagttgataatgacttttcaaaactatgggcaagctttgtttaaaccaatgaaacaaacgagagaacaagaaactccacctgattttttttatttttcggattatgaaagacataatgctgaaattgctgctttccacttggacagaatattggattttcgcagagttccacctgttgctggtcggatggttaacatgactaaggaaattagagatgttactagagataaaaaattgtggagaacttttttcatttctccggctaataatatttgtttttacggggaatgttcttattattgttctactgaacatgctttgtgtggtaaacctgatcaaattgaaggttctttggctgcttttttgcctgatttgtctttggctaaaagaaaaacttggagaaatccatggagaagatcttatcataaaagaaaaaaagctgaatgggaagttgatcctgattattgtgaagaagttaaacaaactccaccatatgattcgtctcatagaatattggacgtcatggatatgacgatctttgactttctgatggggaacatggacagacatcactatgaaacattcgaaaaattcggtaatgaaacttttatcatccatttggataatggtagaggttttggtaaatattctcatgatgaattgtctattttggttccattgcaacagtgttgtagaataaggaaaagcacttacttaagattacaactcttggctaaagaagaatataaattgtctttgttgatggctgaatctttgagaggtgatcaagttgctcctgttttgtatcaaccacatttggaagctttggatagaagattgagagttgttttgaaagctgttagagattgtgttgaaagaaatggtttgcattctgttgttgatgatgatttggatactgaacatagagctgcttctgctagagattataaagatgatgatgataaataa

SEQ ID NO: 403 - DNA, 1596 bp, synthetic construct
  source 1..1596: mol_type = other DNA
atggttcatataggttttagaagcttgaaagccgttttcattttagcattgtcctcattgattttgtatggtattgttactacttttgatggtgatttttcttctgatccatcttctaatttatctagtcactctttggaaaaattgccacctgctgctgagcctgctgaaagagctttgagaggtagagaccctggtgcgctcagaccacatgatcctgctcatagaccattgttgagagaccctggtcctagaagatctgaatctccacctggtcctggtggtgatgcttctttgttggctagattgtttgaacatccattgtatagagttgctgttccaccattgactgaagaagatgttttgtttaatgttaattctgatactagattgtctccaaaagctgctgaaaatcctgattggccacatgctggtgctgaaggtgctgaatttttgtctcctggtgaagcggctgttgattcctatccaaattggttgaaattccacatcggtattaaccgatacgaattgtattctagacataatcctgctattgaagctttgttgcatgatttgtctagtcagagaattacttctgttgctatgaagtctggtggtactcagttgaagttgataatgacttttcaaaactatgggcaagctttgtttaaaccaatgaaacaaacgagagaacaagaaactccacctgattttttttatttttcggattatgaaagacataatgctgaaattgctgctttccacttggacagaatattggattttcgcagagttccacctgttgctggtcggatggttaacatgactaaggaaattagagatgttactagagataaaaaattgtggagaacttttttcatttctccggctaataatatttgtttttacggggaatgttcttattattgttctactgaacatgctttgtgtggtaaacctgatcaaattgaaggttctttggctgcttttttgcctgatttgtctttggctaaaagaaaaacttggagaaatccatggagaagatcttatcataaaagaaaaaaagctgaatgggaagttgatcctgattattgtgaagaagttaaacaaactccaccatatgattcgtctcatagaatattggacgtcatggatatgacgatctttgactttctgatggggaacatggacagacatcactatgaaacattcgaaaaattcggtaatgaaacttttatcatccatttggataatggtagaggttttggtaaatattctcatgatgaattgtctattttggttccattgcaacagtgttgtagaataaggaaaagcacttacttaagattacaactcttggctaaagaagaatataaattgtctttgttgatggctgaatctttgagaggtgatcaagttgctcctgttttgtatcaaccacatttggaagctttggatagaagattgagagttgttttgaaagctgttagagattgtgttgaaagaaatggtttgcattctgttgttgatgatgatttggatactgaacatagagctgcttctgctagagattataaagatgatgatgataaataa

SEQ ID NO: 404 - DNA, 1755 bp, synthetic construct
  source 1..1755: mol_type = other DNA
atggttcatataggttttagaagcttgaaagccgttttcattttagcattgtcctcattgattttgtatggtattgttactacttttgatggttctagagcttctagatatcaaccaccatatgttaatcattctcaagatccattgtaccattctggtaactcctataatagagaaaacgcgacttttgttaccttgtgtagaaatgaagatttgtattctattatccaatctatcaagaaagtcgaagacgatttttcttctgatccatcttctaatttatctagtcactctttggaaaaattgccacctgctgctgagcctgctgaaagagctttgagaggtagagaccctggtgcgctcagaccacatgatcctgctcatagaccattgttgagagaccctggtcctagaagatctgaatctccacctggtcctggtggtgatgcttctttgttggctagattgtttgaacatccattgtatagagttgctgttccaccattgactgaagaagatgttttgtttaatgttaattctgatactagattgtctccaaaagctgctgaaaatcctgattggccacatgctggtgctgaaggtgctgaatttttgtctcctggtgaagcggctgttgattcctatccaaattggttgaaattccacatcggtattaaccgatacgaattgtattctagacataatcctgctattgaagctttgttgcatgatttgtctagtcagagaattacttctgttgctatgaagtctggtggtactcagttgaagttgataatgacttttcaaaactatgggcaagctttgtttaaaccaatgaaacaaacgagagaacaagaaactccacctgattttttttatttttcggattatgaaagacataatgctgaaattgctgctttccacttggacagaatattggattttcgcagagttccacctgttgctggtcggatggttaacatgactaaggaaattagagatgttactagagataaaaaattgtggagaacttttttcatttctccggctaataatatttgtttttacggggaatgttcttattattgttctactgaacatgctttgtgtggtaaacctgatcaaattgaaggttctttggctgcttttttgcctgatttgtctttggctaaaagaaaaacttggagaaatccatggagaagatcttatcataaaagaaaaaaagctgaatgggaagttgatcctgattattgtgaagaagttaaacaaactccaccatatgattcgtctcatagaatattggacgtcatggatatgacgatctttgactttctgatggggaacatggacagacatcactatgaaacattcgaaaaattcggtaatgaaacttttatcatccatttggataatggtagaggttttggtaaatattctcatgatgaattgtctattttggttccattgcaacagtgttgtagaataaggaaaagcacttacttaagattacaactcttggctaaagaagaatataaattgtctttgttgatggctgaatctttgagaggtgatcaagttgctcctgttttgtatcaaccacatttggaagctttggatagaagattgagagttgttttgaaagctgttagagattgtgttgaaagaaatggtttgcattctgttgttgatgatgatttggatactgaacatagagctgcttctgctagagattataaagatgatgatgataaataa

SEQ ID NO: 405 - DNA, 1953 bp, synthetic construct
  source 1..1953: mol_type = other DNA
atggttcatataggttttagaagcttgaaagccgttttcattttagcattgtcctcattgattttgtatggtattgttactacttttgatggttctagagcttctagatatcaaccaccatatgttaatcattctcaagatccattgtaccattctggtaactcctataatagagaaaacgcgacttttgttaccttgtgtagaaatgaagatttgtattctattatccaatctatcaagaaagtcgaagaccgatttaacaacaaatttgcatacgattgggtttttctgaatgaagttccctttactgatgaatttaaagagaggacttctgttttgatttctggtcaagctaaatatggtttgattccaaaagaacattggtcttatcctgattatattgatcaagaaagagctgctgaatctagaagacaattggaagatcaacatgatttttcttctgatccatcttctaatttatctagtcactctttggaaaaattgccacctgctgctgagcctgctgaaagagctttgagaggtagagaccctggtgcgctcagaccacatgatcctgctcatagaccattgttgagagaccctggtcctagaagatctgaatctccacctggtcctggtggtgatgcttctttgttggctagattgtttgaacatccattgtatagagttgctgttccaccattgactgaagaagatgttttgtttaatgttaattctgatactagattgtctccaaaagctgctgaaaatcctgattggccacatgctggtgctgaaggtgctgaatttttgtctcctggtgaagcggctgttgattcctatccaaattggttgaaattccacatcggtattaaccgatacgaattgtattctagacataatcctgctattgaagctttgttgcatgatttgtctagtcagagaattacttctgttgctatgaagtctggtggtactcagttgaagttgataatgacttttcaaaactatgggcaagctttgtttaaaccaatgaaacaaacgagagaacaagaaactccacctgattttttttatttttcggattatgaaagacataatgctgaaattgctgctttccacttggacagaatattggattttcgcagagttccacctgttgctggtcggatggttaacatgactaaggaaattagagatgttactagagataaaaaattgtggagaacttttttcatttctccggctaataatatttgtttttacggggaatgttcttattattgttctactgaacatgctttgtgtggtaaacctgatcaaattgaaggttctttggctgcttttttgcctgatttgtctttggctaaaagaaaaacttggagaaatccatggagaagatcttatcataaaagaaaaaaagctgaatgggaagttgatcctgattattgtgaagaagttaaacaaactccaccatatgattcgtctcatagaatattggacgtcatggatatgacgatctttgactttctgatggggaacatggacagacatcactatgaaacattcgaaaaattcggtaatgaaacttttatcatccatttggataatggtagaggttttggtaaatattctcatgatgaattgtctattttggttccattgcaacagtgttgtagaataaggaaaagcacttacttaagattacaactcttggctaaagaagaatataaattgtctttgttgatggctgaatctttgagaggtgatcaagttgctcctgttttgtatcaaccacatttggaagctttggatagaagattgagagttgttttgaaagctgttagagattgtgttgaaagaaatggtttgcattctgttgttgatgatgatttggatactgaacatagagctgcttctgctagagattataaagatgatgatgataaataa

SEQ ID NO: 406 - DNA, 1611 bp, synthetic construct
  source 1..1611: mol_type = other DNA
atgttgctaactaaacgtttctctaagttgttcaagttgacgttcattgttttgatactctgtggtttgtttgttatcactaataaatacatggatgaaaatacttctgatttttcttctgatccatcttctaatttatctagtcactctttggaaaaattgccacctgctgctgagcctgctgaaagagctttgagaggtagagaccctggtgcgctcagaccacatgatcctgctcatagaccattgttgagagaccctggtcctagaagatctgaatctccacctggtcctggtggtgatgcttctttgttggctagattgtttgaacatccattgtatagagttgctgttccaccattgactgaagaagatgttttgtttaatgttaattctgatactagattgtctccaaaagctgctgaaaatcctgattggccacatgctggtgctgaaggtgctgaatttttgtctcctggtgaagcggctgttgattcctatccaaattggttgaaattccacatcggtattaaccgatacgaattgtattctagacataatcctgctattgaagctttgttgcatgatttgtctagtcagagaattacttctgttgctatgaagtctggtggtactcagttgaagttgataatgacttttcaaaactatgggcaagctttgtttaaaccaatgaaacaaacgagagaacaagaaactccacctgattttttttatttttcggattatgaaagacataatgctgaaattgctgctttccacttggacagaatattggattttcgcagagttccacctgttgctggtcggatggttaacatgactaaggaaattagagatgttactagagataaaaaattgtggagaacttttttcatttctccggctaataatatttgtttttacggggaatgttcttattattgttctactgaacatgctttgtgtggtaaacctgatcaaattgaaggttctttggctgcttttttgcctgatttgtctttggctaaaagaaaaacttggagaaatccatggagaagatcttatcataaaagaaaaaaagctgaatgggaagttgatcctgattattgtgaagaagttaaacaaactccaccatatgattcgtctcatagaatattggacgtcatggatatgacgatctttgactttctgatggggaacatggacagacatcactatgaaacattcgaaaaattcggtaatgaaacttttatcatccatttggataatggtagaggttttggtaaatattctcatgatgaattgtctattttggttccattgcaacagtgttgtagaataaggaaaagcacttacttaagattacaactcttggctaaagaagaatataaattgtctttgttgatggctgaatctttgagaggtgatcaagttgctcctgttttgtatcaaccacatttggaagctttggatagaagattgagagttgttttgaaagctgttagagattgtgttgaaagaaatggtttgcattctgttgttgatgatgatttggatactgaacatagagctgcttctgctagagattataaagatgatgatgataaataa

SEQ ID NO: 407 - DNA, 1794 bp, synthetic construct
  source 1..1794: mol_type = other DNA
atgttgctaactaaacgtttctctaagttgttcaagttgacgttcattgttttgatactctgtggtttgtttgttatcactaataaatacatggatgaaaatacttctgtaaaggagtataaagagtacctagacagatatgtacagtcttattccaataaatattcttcttcttctgatgctgcttctgctgatgattctactcccttgcgagacaatgatgaagccggtaacgaaaaactcaaatccttttacaataatgtttttaactttttgatggttgattcaccagatttttcttctgatccatcttctaatttatctagtcactctttggaaaaattgccacctgctgctgagcctgctgaaagagctttgagaggtagagaccctggtgcgctcagaccacatgatcctgctcatagaccattgttgagagaccctggtcctagaagatctgaatctccacctggtcctggtggtgatgcttctttgttggctagattgtttgaacatccattgtatagagttgctgttccaccattgactgaagaagatgttttgtttaatgttaattctgatactagattgtctccaaaagctgctgaaaatcctgattggccacatgctggtgctgaaggtgctgaatttttgtctcctggtgaagcggctgttgattcctatccaaattggttgaaattccacatcggtattaaccgatacgaattgtattctagacataatcctgctattgaagctttgttgcatgatttgtctagtcagagaattacttctgttgctatgaagtctggtggtactcagttgaagttgataatgacttttcaaaactatgggcaagctttgtttaaaccaatgaaacaaacgagagaacaagaaactccacctgattttttttatttttcggattatgaaagacataatgctgaaattgctgctttccacttggacagaatattggattttcgcagagttccacctgttgctggtcggatggttaacatgactaaggaaattagagatgttactagagataaaaaattgtggagaacttttttcatttctccggctaataatatttgtttttacggggaatgttcttattattgttctactgaacatgctttgtgtggtaaacctgatcaaattgaaggttctttggctgcttttttgcctgatttgtctttggctaaaagaaaaacttggagaaatccatggagaagatcttatcataaaagaaaaaaagctgaatgggaagttgatcctgattattgtgaagaagttaaacaaactccaccatatgattcgtctcatagaatattggacgtcatggatatgacgatctttgactttctgatggggaacatggacagacatcactatgaaacattcgaaaaattcggtaatgaaacttttatcatccatttggataatggtagaggttttggtaaatattctcatgatgaattgtctattttggttccattgcaacagtgttgtagaataaggaaaagcacttacttaagattacaactcttggctaaagaagaatataaattgtctttgttgatggctgaatctttgagaggtgatcaagttgctcctgttttgtatcaaccacatttggaagctttggatagaagattgagagttgttttgaaagctgttagagattgtgttgaaagaaatggtttgcattctgttgttgatgatgatttggatactgaacatagagctgcttctgctagagattataaagatgatgatgataaataa

SEQ ID NO: 408 - DNA, 1953 bp, synthetic construct
  source 1..1953: mol_type = other DNA
atgttgctaactaaacgtttctctaagttgttcaagttgacgttcattgttttgatactctgtggtttgtttgttatcactaataaatacatggatgaaaatacttctgtaaaggagtataaagagtacctagacagatatgtacagtcttattccaataaatattcttcttcttctgatgctgcttctgctgatgattctactcccttgcgagacaatgatgaagccggtaacgaaaaactcaaatccttttacaataatgtttttaactttttgatggttgattcaccaaaaggttctactgctaaacaatataatgaagcttgtttgttgaaaggtgatattggagatagacctgatcattataaggatttgtacaaattgtctgctaaagaattgtctaaatgtttggaattgtctcctgatgaagttgcttctttgactaaatctgatttttcttctgatccatcttctaatttatctagtcactctttggaaaaattgccacctgctgctgagcctgctgaaagagctttgagaggtagagaccctggtgcgctcagaccacatgatcctgctcatagaccattgttgagagaccctggtcctagaagatctgaatctccacctggtcctggtggtgatgcttctttgttggctagattgtttgaacatccattgtatagagttgctgttccaccattgactgaagaagatgttttgtttaatgttaattctgatactagattgtctccaaaagctgctgaaaatcctgattggccacatgctggtgctgaaggtgctgaatttttgtctcctggtgaagcggctgttgattcctatccaaattggttgaaattccacatcggtattaaccgatacgaattgtattctagacataatcctgctattgaagctttgttgcatgatttgtctagtcagagaattacttctgttgctatgaagtctggtggtactcagttgaagttgataatgacttttcaaaactatgggcaagctttgtttaaaccaatgaaacaaacgagagaacaagaaactccacctgattttttttatttttcggattatgaaagacataatgctgaaattgctgctttccacttggacagaatattggattttcgcagagttccacctgttgctggtcggatggttaacatgactaaggaaattagagatgttactagagataaaaaattgtggagaacttttttcatttctccggctaataatatttgtttttacggggaatgttcttattattgttctactgaacatgctttgtgtggtaaacctgatcaaattgaaggttctttggctgcttttttgcctgatttgtctttggctaaaagaaaaacttggagaaatccatggagaagatcttatcataaaagaaaaaaagctgaatgggaagttgatcctgattattgtgaagaagttaaacaaactccaccatatgattcgtctcatagaatattggacgtcatggatatgacgatctttgactttctgatggggaacatggacagacatcactatgaaacattcgaaaaattcggtaatgaaacttttatcatccatttggataatggtagaggttttggtaaatattctcatgatgaattgtctattttggttccattgcaacagtgttgtagaataaggaaaagcacttacttaagattacaactcttggctaaagaagaatataaattgtctttgttgatggctgaatctttgagaggtgatcaagttgctcctgttttgtatcaaccacatttggaagctttggatagaagattgagagttgttttgaaagctgttagagattgtgttgaaagaaatggtttgcattctgttgttgatgatgatttggatactgaacatagagctgcttctgctagagattataaagatgatgatgataaataa

SEQ ID NO: 409 - DNA, 1629 bp, synthetic construct
  source 1..1629: mol_type = other DNA
atgttggctttgagaagatttatcttgaatcagagatctttgagatcttgtactataccaatattggttggggctttgattattattttggttttgtttcaattggtcactcatagaaacgatgctgatttttcttctgatccatcttctaatttatctagtcactctttggaaaaattgccacctgctgctgagcctgctgaaagagctttgagaggtagagaccctggtgcgctcagaccacatgatcctgctcatagaccattgttgagagaccctggtcctagaagatctgaatctccacctggtcctggtggtgatgcttctttgttggctagattgtttgaacatccattgtatagagttgctgttccaccattgactgaagaagatgttttgtttaatgttaattctgatactagattgtctccaaaagctgctgaaaatcctgattggccacatgctggtgctgaaggtgctgaatttttgtctcctggtgaagcggctgttgattcctatccaaattggttgaaattccacatcggtattaaccgatacgaattgtattctagacataatcctgctattgaagctttgttgcatgatttgtctagtcagagaattacttctgttgctatgaagtctggtggtactcagttgaagttgataatgacttttcaaaactatgggcaagctttgtttaaaccaatgaaacaaacgagagaacaagaaactccacctgattttttttatttttcggattatgaaagacataatgctgaaattgctgctttccacttggacagaatattggattttcgcagagttccacctgttgctggtcggatggttaacatgactaaggaaattagagatgttactagagataaaaaattgtggagaacttttttcatttctccggctaataatatttgtttttacggggaatgttcttattattgttctactgaacatgctttgtgtggtaaacctgatcaaattgaaggttctttggctgcttttttgcctgatttgtctttggctaaaagaaaaacttggagaaatccatggagaagatcttatcataaaagaaaaaaagctgaatgggaagttgatcctgattattgtgaagaagttaaacaaactccaccatatgattcgtctcatagaatattggacgtcatggatatgacgatctttgactttctgatggggaacatggacagacatcactatgaaacattcgaaaaattcggtaatgaaacttttatcatccatttggataatggtagaggttttggtaaatattctcatgatgaattgtctattttggttccattgcaacagtgttgtagaataaggaaaagcacttacttaagattacaactcttggctaaagaagaatataaattgtctttgttgatggctgaatctttgagaggtgatcaagttgctcctgttttgtatcaaccacatttggaagctttggatagaagattgagagttgttttgaaagctgttagagattgtgttgaaagaaatggtttgcattctgttgttgatgatgatttggatactgaacatagagctgcttctgctagagattataaagatgatgatgataaataa

SEQ ID NO: 410 - DNA, 1782 bp, synthetic construct
  source 1..1782: mol_type = other DNA
atgttggctttgagaagatttatcttgaatcagagatctttgagatcttgtactataccaatattggttggggctttgattattattttggttttgtttcaattggtcactcatagaaacgatgctctcattagatcctctaatgttaattctaccaacaaaaaaacactcaaggatgctgatccaaaagttttgattgaagcttttggttctcctgaagttgatcctgttgatactattcctgtttctccacttgaattggtgccattttacgatcaagatttttcttctgatccatcttctaatttatctagtcactctttggaaaaattgccacctgctgctgagcctgctgaaagagctttgagaggtagagaccctggtgcgctcagaccacatgatcctgctcatagaccattgttgagagaccctggtcctagaagatctgaatctccacctggtcctggtggtgatgcttctttgttggctagattgtttgaacatccattgtatagagttgctgttccaccattgactgaagaagatgttttgtttaatgttaattctgatactagattgtctccaaaagctgctgaaaatcctgattggccacatgctggtgctgaaggtgctgaatttttgtctcctggtgaagcggctgttgattcctatccaaattggttgaaattccacatcggtattaaccgatacgaattgtattctagacataatcctgctattgaagctttgttgcatgatttgtctagtcagagaattacttctgttgctatgaagtctggtggtactcagttgaagttgataatgacttttcaaaactatgggcaagctttgtttaaaccaatgaaacaaacgagagaacaagaaactccacctgattttttttatttttcggattatgaaagacataatgctgaaattgctgctttccacttggacagaatattggattttcgcagagttccacctgttgctggtcggatggttaacatgactaaggaaattagagatgttactagagataaaaaattgtggagaacttttttcatttctccggctaataatatttgtttttacggggaatgttcttattattgttctactgaacatgctttgtgtggtaaacctgatcaaattgaaggttctttggctgcttttttgcctgatttgtctttggctaaaagaaaaacttggagaaatccatggagaagatcttatcataaaagaaaaaaagctgaatgggaagttgatcctgattattgtgaagaagttaaacaaactccaccatatgattcgtctcatagaatattggacgtcatggatatgacgatctttgactttctgatggggaacatggacagacatcactatgaaacattcgaaaaattcggtaatgaaacttttatcatccatttggataatggtagaggttttggtaaatattctcatgatgaattgtctattttggttccattgcaacagtgttgtagaataaggaaaagcacttacttaagattacaactcttggctaaagaagaatataaattgtctttgttgatggctgaatctttgagaggtgatcaagttgctcctgttttgtatcaaccacatttggaagctttggatagaagattgagagttgttttgaaagctgttagagattgtgttgaaagaaatggtttgcattctgttgttgatgatgatttggatactgaacatagagctgcttctgctagagattataaagatgatgatgataaataa

SEQ ID NO: 411 - DNA, 1962 bp, synthetic construct
  source 1..1962: mol_type = other DNA
atgttggctttgagaagatttatcttgaatcagagatctttgagatcttgtactataccaatattggttggggctttgattattattttggttttgtttcaattggtcactcatagaaacgatgctctcattagatcctctaatgttaattctaccaacaaaaaaacactcaaggatgctgatccaaaagttttgattgaagcttttggttctcctgaagttgatcctgttgatactattcctgtttctccacttgaattggtgccattttacgatcaatctattgatactaagaggtcctcttcatggttgataaacaaaaagggttattataaacacttcaacgaactgtctttgacggacagatgcaagttctattttagaacattgtatactctagacgatgagtggactaactctgttaaaaaattggaatattcaattaatgataatgaaggtgatttttcttctgatccatcttctaatttatctagtcactctttggaaaaattgccacctgctgctgagcctgctgaaagagctttgagaggtagagaccctggtgcgctcagaccacatgatcctgctcatagaccattgttgagagaccctggtcctagaagatctgaatctccacctggtcctggtggtgatgcttctttgttggctagattgtttgaacatccattgtatagagttgctgttccaccattgactgaagaagatgttttgtttaatgttaattctgatactagattgtctccaaaagctgctgaaaatcctgattggccacatgctggtgctgaaggtgctgaatttttgtctcctggtgaagcggctgttgattcctatccaaattggttgaaattccacatcggtattaaccgatacgaattgtattctagacataatcctgctattgaagctttgttgcatgatttgtctagtcagagaattacttctgttgctatgaagtctggtggtactcagttgaagttgataatgacttttcaaaactatgggcaagctttgtttaaaccaatgaaacaaacgagagaacaagaaactccacctgattttttttatttttcggattatgaaagacataatgctgaaattgctgctttccacttggacagaatattggattttcgcagagttccacctgttgctggtcggatggttaacatgactaaggaaattagagatgttactagagataaaaaattgtggagaacttttttcatttctccggctaataatatttgtttttacggggaatgttcttattattgttctactgaacatgctttgtgtggtaaacctgatcaaattgaaggttctttggctgcttttttgcctgatttgtctttggctaaaagaaaaacttggagaaatccatggagaagatcttatcataaaagaaaaaaagctgaatgggaagttgatcctgattattgtgaagaagttaaacaaactccaccatatgattcgtctcatagaatattggacgtcatggatatgacgatctttgactttctgatggggaacatggacagacatcactatgaaacattcgaaaaattcggtaatgaaacttttatcatccatttggataatggtagaggttttggtaaatattctcatgatgaattgtctattttggttccattgcaacagtgttgtagaataaggaaaagcacttacttaagattacaactcttggctaaagaagaatataaattgtctttgttgatggctgaatctttgagaggtgatcaagttgctcctgttttgtatcaaccacatttggaagctttggatagaagattgagagttgttttgaaagctgttagagattgtgttgaaagaaatggtttgcattctgttgttgatgatgatttggatactgaacatagagctgcttctgctagagattataaagatgatgatgataaataa

SEQ ID NO: 412 - DNA, 1593 bp, synthetic construct
  source 1..1593: mol_type = other DNA
atgcatgttttgttgtctaaaaagattgctagatttttgttgatatcttttgtttttgttttggctttgatggttaccattaaccatccggatttttcttctgatccatcttctaatttatctagtcactctttggaaaaattgccacctgctgctgagcctgctgaaagagctttgagaggtagagaccctggtgcgctcagaccacatgatcctgctcatagaccattgttgagagaccctggtcctagaagatctgaatctccacctggtcctggtggtgatgcttctttgttggctagattgtttgaacatccattgtatagagttgctgttccaccattgactgaagaagatgttttgtttaatgttaattctgatactagattgtctccaaaagctgctgaaaatcctgattggccacatgctggtgctgaaggtgctgaatttttgtctcctggtgaagcggctgttgattcctatccaaattggttgaaattccacatcggtattaaccgatacgaattgtattctagacataatcctgctattgaagctttgttgcatgatttgtctagtcagagaattacttctgttgctatgaagtctggtggtactcagttgaagttgataatgacttttcaaaactatgggcaagctttgtttaaaccaatgaaacaaacgagagaacaagaaactccacctgattttttttatttttcggattatgaaagacataatgctgaaattgctgctttccacttggacagaatattggattttcgcagagttccacctgttgctggtcggatggttaacatgactaaggaaattagagatgttactagagataaaaaattgtggagaacttttttcatttctccggctaataatatttgtttttacggggaatgttcttattattgttctactgaacatgctttgtgtggtaaacctgatcaaattgaaggttctttggctgcttttttgcctgatttgtctttggctaaaagaaaaacttggagaaatccatggagaagatcttatcataaaagaaaaaaagctgaatgggaagttgatcctgattattgtgaagaagttaaacaaactccaccatatgattcgtctcatagaatattggacgtcatggatatgacgatctttgactttctgatggggaacatggacagacatcactatgaaacattcgaaaaattcggtaatgaaacttttatcatccatttggataatggtagaggttttggtaaatattctcatgatgaattgtctattttggttccattgcaacagtgttgtagaataaggaaaagcacttacttaagattacaactcttggctaaagaagaatataaattgtctttgttgatggctgaatctttgagaggtgatcaagttgctcctgttttgtatcaaccacatttggaagctttggatagaagattgagagttgttttgaaagctgttagagattgtgttgaaagaaatggtttgcattctgttgttgatgatgatttggatactgaacatagagctgcttctgctagagattataaagatgatgatgataaataa

SEQ ID NO: 413 - DNA, 1758 bp, synthetic construct
  source 1..1758: mol_type = other DNA
atgcatgttttgttgtctaaaaagattgctagatttttgttgatatcttttgtttttgttttggctttgatggttaccattaaccatccgaagactaaacaaatgtctgaacaatatgttactccatatttgccaaaatctttgcaaccaattgctaaaatttctgctgaagaacaaagaagaattcaatctgaacaagaagaagctgaattgaaacaatctttggaaggtgaagcaataagaaatgctaccgttgatttttcttctgatccatcttctaatttatctagtcactctttggaaaaattgccacctgctgctgagcctgctgaaagagctttgagaggtagagaccctggtgcgctcagaccacatgatcctgctcatagaccattgttgagagaccctggtcctagaagatctgaatctccacctggtcctggtggtgatgcttctttgttggctagattgtttgaacatccattgtatagagttgctgttccaccattgactgaagaagatgttttgtttaatgttaattctgatactagattgtctccaaaagctgctgaaaatcctgattggccacatgctggtgctgaaggtgctgaatttttgtctcctggtgaagcggctgttgattcctatccaaattggttgaaattccacatcggtattaaccgatacgaattgtattctagacataatcctgctattgaagctttgttgcatgatttgtctagtcagagaattacttctgttgctatgaagtctggtggtactcagttgaagttgataatgacttttcaaaactatgggcaagctttgtttaaaccaatgaaacaaacgagagaacaagaaactccacctgattttttttatttttcggattatgaaagacataatgctgaaattgctgctttccacttggacagaatattggattttcgcagagttccacctgttgctggtcggatggttaacatgactaaggaaattagagatgttactagagataaaaaattgtggagaacttttttcatttctccggctaataatatttgtttttacggggaatgttcttattattgttctactgaacatgctttgtgtggtaaacctgatcaaattgaaggttctttggctgcttttttgcctgatttgtctttggctaaaagaaaaacttggagaaatccatggagaagatcttatcataaaagaaaaaaagctgaatgggaagttgatcctgattattgtgaagaagttaaacaaactccaccatatgattcgtctcatagaatattggacgtcatggatatgacgatctttgactttctgatggggaacatggacagacatcactatgaaacattcgaaaaattcggtaatgaaacttttatcatccatttggataatggtagaggttttggtaaatattctcatgatgaattgtctattttggttccattgcaacagtgttgtagaataaggaaaagcacttacttaagattacaactcttggctaaagaagaatataaattgtctttgttgatggctgaatctttgagaggtgatcaagttgctcctgttttgtatcaaccacatttggaagctttggatagaagattgagagttgttttgaaagctgttagagattgtgttgaaagaaatggtttgcattctgttgttgatgatgatttggatactgaacatagagctgcttctgctagagattataaagatgatgatgataaataa

SEQ ID NO: 414 - DNA, 1983 bp, synthetic construct
  source 1..1983: mol_type = other DNA
atgcatgttttgttgtctaaaaagattgctagatttttgttgatatcttttgtttttgttttggctttgatggttaccattaaccatccgaagactaaacaaatgtctgaacaatatgttactccatatttgccaaaatctttgcaaccaattgctaaaatttctgctgaagaacaaagaagaattcaatctgaacaagaagaagctgaattgaaacaatctttggaaggtgaagcaataagaaatgctaccgttaacgccattaaagaaaaaattaaatcttatggtggtaatgaaactactttgggttttatggttccatcttatattaatcatagaggttctccacccaaagcttgcttcgtttcattgatcacagaaagggactctatgactcaaatcttgcaatctatagatgaggtccaagtcaagtttaacaaaaattttgcttatccatgggtttttatttctcaaggtgaagatttttcttctgatccatcttctaatttatctagtcactctttggaaaaattgccacctgctgctgagcctgctgaaagagctttgagaggtagagaccctggtgcgctcagaccacatgatcctgctcatagaccattgttgagagaccctggtcctagaagatctgaatctccacctggtcctggtggtgatgcttctttgttggctagattgtttgaacatccattgtatagagttgctgttccaccattgactgaagaagatgttttgtttaatgttaattctgatactagattgtctccaaaagctgctgaaaatcctgattggccacatgctggtgctgaaggtgctgaatttttgtctcctggtgaagcggctgttgattcctatccaaattggttgaaattccacatcggtattaaccgatacgaattgtattctagacataatcctgctattgaagctttgttgcatgatttgtctagtcagagaattacttctgttgctatgaagtctggtggtactcagttgaagttgataatgacttttcaaaactatgggcaagctttgtttaaaccaatgaaacaaacgagagaacaagaaactccacctgattttttttatttttcggattatgaaagacataatgctgaaattgctgctttccacttggacagaatattggattttcgcagagttccacctgttgctggtcggatggttaacatgactaaggaaattagagatgttactagagataaaaaattgtggagaacttttttcatttctccggctaataatatttgtttttacggggaatgttcttattattgttctactgaacatgctttgtgtggtaaacctgatcaaattgaaggttctttggctgcttttttgcctgatttgtctttggctaaaagaaaaacttggagaaatccatggagaagatcttatcataaaagaaaaaaagctgaatgggaagttgatcctgattattgtgaagaagttaaacaaactccaccatatgattcgtctcatagaatattggacgtcatggatatgacgatctttgactttctgatggggaacatggacagacatcactatgaaacattcgaaaaattcggtaatgaaacttttatcatccatttggataatggtagaggttttggtaaatattctcatgatgaattgtctattttggttccattgcaacagtgttgtagaataaggaaaagcacttacttaagattacaactcttggctaaagaagaatataaattgtctttgttgatggctgaatctttgagaggtgatcaagttgctcctgttttgtatcaaccacatttggaagctttggatagaagattgagagttgttttgaaagctgttagagattgtgttgaaagaaatggtttgcattctgttgttgatgatgatttggatactgaacatagagctgcttctgctagagattataaagatgatgatgataaataa

SEQ ID NO: 415 - DNA, 1023 bp, synthetic construct
  source 1..1023: mol_type = other DNA
tttatctttttttagtatagagtttgtgtgtttaaagcttgtttatgtttcaattgaaactattgtttttgcttgatgatgatgtagggcaaaacagaaacaacattgtacaaatggatatagagagacctactacaaaagtggatggattgtcagaatcgaccgggtgtttatatacatattccagaagaccaaatggctggtctaattgtatacaagtaagacagctgatttctagctggtgtgaaaatagcacactttgagaaaaatacataccaccctgcaacaggtgttaaccgtttaagacctagaaataaccgcagtatataaagatcaatcgaatacttgggttggtgaaaagcaacccaaaatacatcatctgctcatatgattgtgatctgattgtgaatagatcaatacactactagaatagaagaacatcacacgctatttttttttcctcttctgtcctactcggagaaacagaaagtatcaggtcaccaccggacatgtcatactacctcggaagattcccaattacgcacgtaaaaactaaaacgagcccccaccaaagaacaaaaaagaaggtgctgggcccccactttcttcccttgcacgtgataggaagatggctacagaaacaagaagatggaaatcgaaggaaagagggagactggaagctgtaaaaactgaaatgaaaaaaaaaaaaaaaaaaaaaaacaagaagctgaaaatggaagactgaaatttgaaaaatggtaaaaaaaaaaaagaaacacgaagctaaaaacctggattccattttgagaagaagcaagaaaggtaagtatggtaacgaccgtacaggcaagcgcgaaggcaaatggaaaagctggagtccggaagataatcatttcatcttcttttgttagaacagaacagtggatgtccctcatctcggtaacgtattgtccatgccctagaactctctgtccctaaaaagaggacaaaaacccaatggtttccccagcttccagtggagcc

SEQ ID NO: 416 - protein, 214 aa, synthetic construct
MKLLILTCLVAVALARPKHPIKHQGLPQEVLNENLLRFFVAPFPEVFGKEKVNELSKDIGSESTEDQAMEDIKQMEAESISSSEEIVPNSVEQKHIQKEDVPSERYLGYLEQLLRLKKYKVPQLEIVPNSAEERLHSMKEGIHAQQKEPMIGVNQELAYFYPELFRQFYQLDAYPSGAWYYVPLGTQYTDAPSFSDIPNPIGSENSGKTTMPLW

SEQ ID NO: 417 - protein, 222 aa, synthetic construct
MKFFIFTCLLAVALAKNTMEHVSSSEESIISQETYKQEKNMAINPSKENLCSTFCKEVVRNANEEEYSIGSSSEESAEVATEEVKITVDDKHYQKALNEINQFYQKFPQYLQYLYQGPIVLNPWDQVKRNAVPITPTLNREQLSTSEENSKKTVDMESTEVFTKKTKLTEEEKNRLNFLKKISQRYQKFALPQYLKTVYQHQKAMKPWIQPKTKVIPYVRYL

SEQ ID NO: 418 - protein, 224 aa, synthetic construct
MKVLILACLVALALARELEELNVPGEIVESLSSSEESITRINKKIEKFQSEEQQQTEDELQDKIHPFAQTQSLVYPFPGPIPNSLPQNIPPLTQTPVVVPPFLQPEVMGVSKVKEAMAPKHKEMPFPKYPVEPFTESQSLTLTDVENLHLPLPLLQSWMHQPHQPLPPTVMFPPQSVLSLSQSKVLPVPQKAVPYPQRDMPIQAFLLYQEPVLGPVRGPFPIIV

SEQ ID NO: 419 - protein, 190 aa, synthetic construct
MMKSFFLVVTILALTLPFLGAQEQNQEQPIRCEKDERFFSDKIAKYIPIQYVLSRYPSYGLNYYQQKPVALINNQFLPYPYYAKPAAVRSPAQILQWQVLSNTVPAKSCQAQPTTMARHPHPHLSFMAIPPKKNQDKTEIPTINTIASGEPTSTPTIEAVESTVATLEASPEVIESPPEINTVQVTSTAV

SEQ ID NO: 420 - protein, 214 aa, synthetic construct
MKLLILTCLVAVALARPKHPIKHQGLPQEVLNENLLRFFVAPFPEVFGKEKVNELSKDIGSESTEDQAMEDIKQMEAESISSSEEIVPNSVEQKHIQKEDVPSERYLGYLEQLLRLKKYKVPQLEIVPNSAEERLHSMKEGIHAQQKEPMIGVNQELAYFYPELFRQFYQLDAYPSGAWYYVPLGTQYTDAPSFSDIPNPIGSENSEKTTMPLW

SEQ ID NO: 421 - protein, 314 aa, synthetic construct
MRIAVICFCLLGITCAIPVKQADSGSSEEKQLYNKYPDAVATWLNPDPSQKQNLLAPQNAVSSEETNDFKQETLPSKSNESHDHMDDMDDEDDDDHVDSQDSIDSNDSDDVDDTDDSHQSDESHHSDESDELVTDFPTDLPATEVFTPVVPTVDTYDGRGDSVVYGLRSKSKKFRRPDIQYPDATDEDITSHMESEELNGAYKAIPVAQDLNAPSDWDSRGKDSYETSQLDDQSAETHSHKQSRLYKRKANDESNEHSDVIDSQELSKVSREFHSHEFHSHEDMLVVDPKSKEEDKHLKFRISHELDSASSEVN

SEQ ID NO: 422 - protein, 287 aa, synthetic construct
MRQVWFSWIVGLFLCFFNVSSAAPVNTTTEDETAQIPAEAVIGYSDLEGDFDVAVLPFSNSTNNGLLFINTTIASIAAKEEGVSLDKRRPKHPIKHQGLPQEVLNENLLRFFVAPFPEVFGKEKVNELSKDIGSESTEDQAMEDIKQMEAESISSSEEIVPNSVEQKHIQKEDVPSERYLGYLEQLLRLKKYKVPQLEIVPNSAEERLHSMKEGIHAQQKEPMIGVNQELAYFYPELFRQFYQLDAYPSGAWYYVPLGTQYTDAPSFSDIPNPIGSENSGKTTMPLW

SEQ ID NO: 423 - protein, 295 aa, synthetic construct
MRQVWFSWIVGLFLCFFNVSSAAPVNTTTEDETAQIPAEAVIGYSDLEGDFDVAVLPFSNSTNNGLLFINTTIASIAAKEEGVSLDKRKNTMEHVSSSEESIISQETYKQEKNMAINPSKENLCSTFCKEVVRNANEEEYSIGSSSEESAEVATEEVKITVDDKHYQKALNEINQFYQKFPQYLQYLYQGPIVLNPWDQVKRNAVPITPTLNREQLSTSEENSKKTVDMESTEVFTKKTKLTEEEKNRLNFLKKISQRYQKFALPQYLKTVYQHQKAMKPWIQPKTKVIPYVRYL

SEQ ID NO: 424 - protein, 297 aa, synthetic construct
MRQVWFSWIVGLFLCFFNVSSAAPVNTTTEDETAQIPAEAVIGYSDLEGDFDVAVLPFSNSTNNGLLFINTTIASIAAKEEGVSLDKRRELEELNVPGEIVESLSSSEESITRINKKIEKFQSEEQQQTEDELQDKIHPFAQTQSLVYPFPGPIPNSLPQNIPPLTQTPVVVPPFLQPEVMGVSKVKEAMAPKHKEMPFPKYPVEPFTESQSLTLTDVENLHLPLPLLQSWMHQPHQPLPPTVMFPPQSVLSLSQSKVLPVPQKAVPYPQRDMPIQAFLLYQEPVLGPVRGPFPIIV

SEQ ID NO: 425 - protein, 257 aa, synthetic construct
MRQVWFSWIVGLFLCFFNVSSAAPVNTTTEDETAQIPAEAVIGYSDLEGDFDVAVLPFSNSTNNGLLFINTTIASIAAKEEGVSLDKRQEQNQEQPIRCEKDERFFSDKIAKYIPIQYVLSRYPSYGLNYYQQKPVALINNQFLPYPYYAKPAAVRSPAQILQWQVLSNTVPAKSCQAQPTTMARHPHPHLSFMAIPPKKNQDKTEIPTINTIASGEPTSTPTIEAVESTVATLEASPEVIESPPEINTVQVTSTAV

SEQ ID NO: 426 - protein, 88 aa, synthetic construct
MRQVWFSWIVGLFLCFFNVSSAAPVNTTTEDETAQIPAEAVIGYSDLEGDFDVAVLPFSNSTNNGLLFINTTIASIAAKEEGVSLDKR

SEQ ID NO: 427 - DNA, 864 bp, synthetic construct
  source 1..864: mol_type = other DNA
atgaggcaggtttggttctcttggattgtgggattgttcctatgttttttcaacgtgtcttctgctgctccagtcaacactacaacagaagatgaaacggcacaaattccggctgaagctgtcatcggttactcagatttagaaggggatttcgatgttgctgttttgccattttccaacagcacaaataacgggttattgtttataaatactactattgccagcattgctgctaaagaagaaggggtatctttggataaaagacgaccaaaacatcctatcaagcatcaaggattgcctcaagaagtcttgaacgaaaatttactcaggttttttgtggcaccttttccagaagtctttggaaaggagaaggtcaatgaactgtccaaggacattggtagtgaatcaactgaggatcaagcaatggaagatattaagcaaatggaagctgaaagcatttcgtcatccgaggaaattgttcccaatagcgttgagcagaagcacatacaaaaggaggatgtaccgtctgagcgttacctgggttatctggagcagcttctcagattgaaaaaatacaaagtaccccagttggaaattgttcccaactctgctgaggaaagattgcactccatgaaagagggaatccatgcgcaacagaaagaacctatgataggtgtgaaccaggaacttgcctacttctatcctgagcttttcagacaattctaccaactggatgcctatccatctggtgcatggtattacgttccactagggacacaatataccgacgccccatcattctctgacatcccaaatcctattggctctgagaacagtggtaagactacgatgccattgtggtaa

SEQ ID NO: 428 - DNA, 888 bp, synthetic construct
  source 1..888: mol_type = other DNA
atgaggcaggtttggttctcttggattgtgggattgttcctatgttttttcaacgtgtcttctgctgctccagtcaacactacaacagaagatgaaacggcacaaattccggctgaagctgtcatcggttactcagatttagaaggggatttcgatgttgctgttttgccattttccaacagcacaaataacgggttattgtttataaatactactattgccagcattgctgctaaagaagaaggggtatctttggataaaagaaagaacacgatggaacatgtctcctctagtgaggagtctataatctcgcaagagacatacaagcaggagaagaatatggccattaatccaagcaaggagaacttgtgttccaccttctgcaaggaagtcgtaaggaacgcaaatgaagaggaatactctatcggatcatctagtgaggaatctgctgaggttgccactgaggaagtcaagattactgtggacgataagcactaccaaaaagctctaaacgaaatcaatcagttttatcaaaagtttccccaatatttgcaatatctgtaccaaggtccaattgttttgaacccttgggatcaggttaaaagaaatgctgttccgattactccaactttaaacagagagcaactctccaccagtgaggaaaattcaaaaaaaaccgtggacatggaatcaacagaagtattcactaagaaaacaaaactgacggaagaagaaaagaacagactaaattttttaaaaaaaatcagccaacgttaccagaaatttgcgttgccacagtatctgaaaactgtctatcagcatcaaaaagctatgaagccttggattcaacctaagacaaaggttattccctatgtgcgatacctttaa

SEQ ID NO: 429 - DNA, 894 bp, synthetic construct
  source 1..894: mol_type = other DNA
atgaggcaggtttggttctcttggattgtgggattgttcctatgttttttcaacgtgtcttctgctgctccagtcaacactacaacagaagatgaaacggcacaaattccggctgaagctgtcatcggttactcagatttagaaggggatttcgatgttgctgttttgccattttccaacagcacaaataacgggttattgtttataaatactactattgccagcattgctgctaaagaagaaggggtatctttggataaaagaagagagttggaagaattaaatgtaccaggtgagatagttgaatctctatcaagctcggaggaatcaattacccgtataaacaagaaaattgagaagtttcagagtgaggaacaacagcaaacagaggacgaattgcaagataaaatccatccatttgcgcaaacacagtctctagtctatcccttccctggaccgatccctaactcactcccacaaaacattcctcctttaactcaaacccctgtggttgttccgccattcttgcaaccagaagtaatgggagtttccaaagttaaggaggctatggctcctaagcataaggaaatgcccttcccaaaatatccagttgagccctttactgaaagccagtctctgactcttacggacgttgaaaatttgcaccttccactaccattgttgcagtcttggatgcatcaacctcaccaacctttaccaccaactgtcatgtttcctcctcaatccgtgttgagtctttctcagtccaaagtcttgcctgttccgcagaaagctgtgccatacccacagagagatatgcccattcaagcatttttgctgtaccaagaaccagtattaggtccagttaggggccccttccctattattgtctaa

SEQ ID NO: 430 - DNA, 774 bp, synthetic construct
  source 1..774: mol_type = other DNA
atgaggcaggtttggttctcttggattgtgggattgttcctatgttttttcaacgtgtcttctgctgctccagtcaacactacaacagaagatgaaacggcacaaattccggctgaagctgtcatcggttactcagatttagaaggggatttcgatgttgctgttttgccattttccaacagcacaaataacgggttattgtttataaatactactattgccagcattgctgctaaagaagaaggggtatctttggataaaagacaagaacaaaaccaagaacaacccataagatgtgagaaggatgaaagattcttctctgacaaaatagccaaatatataccaattcagtacgtgctatctaggtatcctagctatggactcaattactaccaacagaaaccagttgctttgattaacaatcaatttcttccatacccctattacgcaaagcccgctgcagttcgatcccctgcgcaaattcttcaatggcaagtcttgtcaaatactgtgcctgctaagtcctgccaagctcagccgacgacgatggctcgtcacccacatccacatttatcttttatggccattccgccaaagaaaaatcaggataaaacagaaatccctaccatcaacaccattgctagtggcgagcctacatccacacctaccattgaagcagtagagtcaactgtagctactttggaagcctctccagaagttattgagtcgccacctgagatcaacacagtccaggttacttcaactgctgtctaa

SEQ ID NO: 431 - DNA, 600 bp, synthetic construct
  source 1..600: mol_type = other DNA
cgaccaaaacatcctatcaagcatcaaggattgcctcaagaagtcttgaacgaaaatttactcaggttttttgtggcaccttttccagaagtctttggaaaggagaaggtcaatgaactgtccaaggacattggtagtgaatcaactgaggatcaagcaatggaagatattaagcaaatggaagctgaaagcatttcgtcatccgaggaaattgttcccaatagcgttgagcagaagcacatacaaaaggaggatgtaccgtctgagcgttacctgggttatctggagcagcttctcagattgaaaaaatacaaagtaccccagttggaaattgttcccaactctgctgaggaaagattgcactccatgaaagagggaatccatgcgcaacagaaagaacctatgataggtgtgaaccaggaacttgcctacttctatcctgagcttttcagacaattctaccaactggatgcctatccatctggtgcatggtattacgttccactagggacacaatataccgacgccccatcattctctgacatcccaaatcctattggctctgagaacagtggtaagactacgatgccattgtggtaa

SEQ ID NO: 432 - DNA, 624 bp, synthetic construct
  source 1..624: mol_type = other DNA
aagaacacgatggaacatgtctcctctagtgaggagtctataatctcgcaagagacatacaagcaggagaagaatatggccattaatccaagcaaggagaacttgtgttccaccttctgcaaggaagtcgtaaggaacgcaaatgaagaggaatactctatcggatcatctagtgaggaatctgctgaggttgccactgaggaagtcaagattactgtggacgataagcactaccaaaaagctctaaacgaaatcaatcagttttatcaaaagtttccccaatatttgcaatatctgtaccaaggtccaattgttttgaacccttgggatcaggttaaaagaaatgctgttccgattactccaactttaaacagagagcaactctccaccagtgaggaaaattcaaaaaaaaccgtggacatggaatcaacagaagtattcactaagaaaacaaaactgacggaagaagaaaagaacagactaaattttttaaaaaaaatcagccaacgttaccagaaatttgcgttgccacagtatctgaaaactgtctatcagcatcaaaaagctatgaagccttggattcaacctaagacaaaggttattccctatgtgcgatacctttaa

SEQ ID NO: 433 - DNA, 630 bp, synthetic construct
  source 1..630: mol_type = other DNA
agagagttggaagaattaaatgtaccaggtgagatagttgaatctctatcaagctcggaggaatcaattacccgtataaacaagaaaattgagaagtttcagagtgaggaacaacagcaaacagaggacgaattgcaagataaaatccatccatttgcgcaaacacagtctctagtctatcccttccctggaccgatccctaactcactcccacaaaacattcctcctttaactcaaacccctgtggttgttccgccattcttgcaaccagaagtaatgggagtttccaaagttaaggaggctatggctcctaagcataaggaaatgcccttcccaaaatatccagttgagccctttactgaaagccagtctctgactcttacggacgttgaaaatttgcaccttccactaccattgttgcagtcttggatgcatcaacctcaccaacctttaccaccaactgtcatgtttcctcctcaatccgtgttgagtctttctcagtccaaagtcttgcctgttccgcagaaagctgtgccatacccacagagagatatgcccattcaagcatttttgctgtaccaagaaccagtattaggtccagttaggggccccttccctattattgtctaa

SEQ ID NO: 434 - DNA, 510 bp, synthetic construct
  source 1..510: mol_type = other DNA
caagaacaaaaccaagaacaacccataagatgtgagaaggatgaaagattcttctctgacaaaatagccaaatatataccaattcagtacgtgctatctaggtatcctagctatggactcaattactaccaacagaaaccagttgctttgattaacaatcaatttcttccatacccctattacgcaaagcccgctgcagttcgatcccctgcgcaaattcttcaatggcaagtcttgtcaaatactgtgcctgctaagtcctgccaagctcagccgacgacgatggctcgtcacccacatccacatttatcttttatggccattccgccaaagaaaaatcaggataaaacagaaatccctaccatcaacaccattgctagtggcgagcctacatccacacctaccattgaagcagtagagtcaactgtagctactttggaagcctctccagaagttattgagtcgccacctgagatcaacacagtccaggttacttcaactgctgtctaa

SEQ ID NO: 435 - protein, 383 aa, synthetic construct
MRFPSIFTAVLFAASSALAAPVNTTTEDETAQIPAEAVIGYSDLEGDFDVAVLPFSNSTNNGLLFINTTIASIAAKEEGVSLEKRIPVKQADSGSSEEKQLYNKYPDAVATWLNPDPSQKQNLLAPQNAVSSEETNDFKQETLPSKSNESHDHMDDMDDEDDDDHVDSQDSIDSNDSDDVDDTDDSHQSDESHHSDESDELVTDFPTDLPATEVFTPVVPTVDTYDGRGDSVVYGLRSKSKKFRRPDIQYPDATDEDITSHMESEELNGAYKAIPVAQDLNAPSDWDSRGKDSYETSQLDDQSAETHSHKQSRLYKRKANDESNEHSDVIDSQELSKVSREFHSHEFHSHEDMLVVDPKSKEEDKHLKFRISHELDSASSEVN

SEQ ID NO: 436 - protein, 7 aa, unidentified
DYDDDDK

SEQ ID NO: 437 - protein, 6 aa, synthetic construct
HHHHHH

SEQ ID NO: 438 - protein, 23 aa, synthetic construct
MDYKDHDGDYKDHDIDYKDDDDK

SEQ ID NO: 439 - protein, 405 aa, synthetic construct; identical to SEQ ID NO: 176
  SITE 77: note = Phosphorylated residue
  SITE 84: note = Phosphorylated residue
  SITE 100: note = Phosphorylated residue
  SITE 107: note = Phosphorylated residue
  SITE 173: note = Phosphorylated residue
  SITE 214: note = Phosphorylated residue
  SITE 221: note = Phosphorylated residue
  SITE 230: note = Phosphorylated residue
  SITE 279: note = Phosphorylated residue
  SITE 353: note = Phosphorylated residue

SEQ ID NO: 440 - protein, 199 aa, synthetic construct; identical to SEQ ID NO: 177
  SITE 41: note = Phosphorylated residue
  SITE 46: note = Phosphorylated residue
  SITE 48: note = Phosphorylated residue
  SITE 49: note = Phosphorylated residue
  SITE 64: note = Phosphorylated residue
  SITE 66: note = Phosphorylated residue
  SITE 67: note = Phosphorylated residue
  SITE 68: note = Phosphorylated residue
  SITE 75: note = Phosphorylated residue
  SITE 88: note = Phosphorylated residue
  SITE 115: note = Phosphorylated residue

SEQ ID NO: 441 - protein, 207 aa, synthetic construct; identical to SEQ ID NO: 178
  SITE 8: note = Phosphorylated residue
  SITE 9: note = Phosphorylated residue
  SITE 10: note = Phosphorylated residue
  SITE 31: note = Phosphorylated residue
  SITE 37: note = Phosphorylated residue
  SITE 56: note = Phosphorylated residue
  SITE 57: note = Phosphorylated residue
  SITE 58: note = Phosphorylated residue
  SITE 61: note = Phosphorylated residue
  SITE 129: note = Phosphorylated residue
  SITE 130: note = Phosphorylated residue
  SITE 131: note = Phosphorylated residue
  SITE 135: note = Phosphorylated residue
  SITE 143: note = Phosphorylated residue
  SITE 144: note = Phosphorylated residue
  SITE 148: note = Phosphorylated residue

SEQ ID NO: 442 - protein, 209 aa, synthetic construct; identical to SEQ ID NO: 179
  SITE 15: note = Phosphorylated residue
  SITE 17: note = Phosphorylated residue
  SITE 18: note = Phosphorylated residue
  SITE 19: note = Phosphorylated residue
  SITE 22: note = Phosphorylated residue
  SITE 24: note = Phosphorylated residue
  SITE 35: note = Phosphorylated residue
  SITE 41: note = Phosphorylated residue